(19) United States
(12) Patent Application Publication
(10) Pub. No.: US 2025/0171461 A1
(43) Pub. Date: May 29, 2025

(54) STIMULATOR OF INTERFERON GENES (STING) MODULATORS, AND COMPOSITIONS AND METHODS THEREOF

(71) Applicants: Geode Therapeutics Inc., Brookline, MA (US); XtalPi, Inc., Shenzhen (CN)

(72) Inventors: Jean Zhao, Brookline, MA (US); Huimin Cheng, Shenzhen (CN); Yina Sun, Beijing (CN); Yayu Chen, Beijing (CN); Qi Wang, Brookline, MA (US); Xiang Y Yu, Acton, MA (US); Musheng Xu, Tianjin (CN); Legang Wang, Tianjin (CN); Xiuyun Li, Tianjin (CN)

(21) Appl. No.: 18/837,176

(22) PCT Filed: Feb. 21, 2023
(86) PCT No.: PCT/US2023/013456

§ 371 Date: Aug. 8, 2024

Related U.S. Application Data
(60) Provisional application No. 63/312,326, filed on Feb. 21, 2022.

(51) Int. Cl. C07D 498/04; A61K 31/4745; A61K 31/5383; A61K 31/5386; A61K 31/542; A61K 31/55; A61K 31/553; C07D 471/04; C07D 513/04; C07D 513/14; C07D 519/00

(52) U.S. Cl. CPC C07D 498/04; A61K 31/4745; A61K 31/5383; A61K 31/5386; A61K 31/542; A61K 31/55; A61K 31/553; C07D 471/04; C07D 513/04; C07D 513/14; C07D 519/00

(57) ABSTRACT

The invention provides novel modulators of STING (Stimulator of Interferon Genes) and pharmaceutical compositions thereof, as well as methods of their preparation and use, in therapy of various diseases and conditions, such as cancer, infectious and autoimmune diseases or disorders.

### TECHNICAL FIELDS OF THE INVENTION

The invention generally relates to pharmaceuticals and therapeutic methods. More particularly, the invention provides novel modulators of Stimulator of Interferon Genes (STING) and pharmaceutical compositions thereof, as well as methods of their preparation and use, in therapy of various diseases and conditions, such as cancer, infectious diseases and autoimmune diseases.

### BACKGROUND OF THE INVENTION

STING, also known as stimulator of interferon genes cGAMP interactor 1 (STING1) encoded by TMEM173, is a signaling molecule associated with the endoplasmic reticulum (ER) and plays a crucial role in managing the transcription of various host defense genes, including pro-inflammatory cytokines and type I interferons (IFNs) in response to aberrant DNA species or cyclic dinucleotides (CDNs) in the cytosol of the cell (Ishikawa H et al., Nature. 2008, 455: 674-678; Ishikawa H et al. Nature. 2009; 461: 788-792; Burdette D et al. Nature. 2011, 478, 515-518).

STING is a 379 amino acid protein with several transmembrane regions and expresses in various endothelial and endothelial cell types, as well as hematopoietic cells, such as T cells, macrophages, and dendritic cells (DCs) (Ishikawa H et al., Nature. 2008, 455: 674-678; Ishikawa H et al. Nature. 2009; 461: 788-792). By stimulating the transcription of innate immune genes in response to some invading bacteria, DNA viruses, or transfected DNA, STING signaling is essential for protecting the cell against a variety of pathogens (Ishikawa H et al., Nature. 2008, 455: 674-678; Ishikawa H et al. Nature. 2009, 461: 788-792; Jin L et al. Mol Cell biol. 2008, 28: 5014-5026; Zhong B et al. Immunity. 2008, 29:538-550; Woo, S. R., et al. Trends Immunol. 2015, 36, 250-256; Barber, G. N. Trends Immunol. 2014, 35, 88-93).

Cytosolic DNA species can trigger STING signaling following binding to and activating the intracellular nucleic acid sensors, such as cyclic GMP-AMP synthase (cGAS), interferon-gamma inducible protein 16 (IF116), and MRE11 homolog, double-strand break repair nuclease (MRE11) (Ablasser A et al. Nature. 2013, 498:380-384; Almine J F et al. Nat Commun. 2017; 8(1):14392; Kondo T et al. Proc Natl Acad Sci USA. 2013; 110:2969-2974). cGAS, a 522 amino acid protein, has been extensively studied among these sensors. cGAS synthesizes an intracellular second messager 2′3′-cyclic GMP-AMP (cGAMP) in response to cytosolic double-stranded DNA (dsDNA) and activates the innate immune STING pathway (Sun L et al. Science. 2013; 339: 786-791; Ablasser A et al. Nature. 2013; 498: 380-384).

Upon binding to CDNs, STING undergoes endoplasmic reticulum (ER)-to-Golgi trafficking and tetramer formation via a higher-order oligomerization, which recruits and activates the downstream TANK-binding kinase 1 (TBK1) and the transcription factor interferon regulatory factor 3 (IRF3) and nuclear factor-κB (NF-κB)(Shang G J et al. Nature. 2019, 567: 389). These transcription factors further translocate into the nucleus to initiate innate immune gene transcription. In addition, STING is associated with sensing aberrant cytosolic DNA species, including self-ssDNA (single-stranded DNA) and dsDNA, with triggering host-defense-related gene expression (Abe T et al. Mol Cell. 2013; 50: 5-15.). STING is then rapidly degraded to avoid problems associated with sustained cytokine production (Shu H B et al. Immunity. 2014; 41: 871-873; Liu Y, et al. N Engl J Med. 2014; 371: 507-518).

The activation of STING induces cytokines such as Type I IFNs, which belong to a family of multiple cytokines, and is involved in antiviral immunity (Zitvogel L et al. Nat Rev Immunol. 2015, 15: 405-414; Gonzalez-Navajas J M et al. Nat Rev Immunol. 2012, 12: 125-135). Upon virus infection, Type I IFNs directly regulate T cell activation, proliferation and survival during T cell priming and protects local T cell expansion against nature killer (NK) mediated attack. Types I IFNs promote functional maturation, migration and antigen presentation of DCs that indirectly affect T cell activation (Tough D F et al. Immunol Cell Biol. 2012, 90: 492-497; Longhi M P et al. J Exp Med. 2009, 206: 1589-1602). The STING signaling pathway can be motivated in macrophages, B cells, some other leukocytes, as well as NK cells, serving as a hub in various innate and adaptive immune responses. (Barber G N. et al. Trends Immunol. 2014; 35: 88-93; Barber G N. et al. Nat Rev Immunol. 2015; 15: 760-770; Sundararaman S K et al. Immunity. 2018; 49: 585-587).

As a crucial element in host antiviral defense pathways, STING takes responsibility against a range of DNA and RNA viruses and bacteria (McNab F et al. Nat. Rev. Immunol. 2015, 15: 87-103). Various viruses, including Herpesviridae, Flaviviridae, Coronaviridae, Papillomaviridae, Adenoviridae, Hepadnaviridae, ortho- and paramyxoviridae, and Rhabdoviridae, have evolved mechanisms to target STING pathways and escape host immune control (Holm C et al., Nat Comm., 2016, 7:10680; Wu J et al., Cell Host Microbe, 2015, 18:333-44; Liu Y et al., J Virol, 2016, 90: 9406-19; Chen X et al. Protein Cell 2014, 5, 369-81; Lau L et al. Science, 2013, 350: 568-71; Ding Q et al. J Hepatol, 2013, 59: 52-8; Nitta S et al. Hepatology, 2013, 57:46-58; Sun L et al. PLoS One, 2012, 7, e30802; Aguirre S et al. PLoS Pathog, 2012, 8, e1002934; Ishikawa H et al. Nature, 2009, vol. 461: 788-92). It is especially worth noting that activation of the STING pathway was reported to block the infection of SARS-CoV-2, a human coronavirus that is the cause of the Covid-19 global pandemic (Liu W, et al. Journal of Virology, 2021, 95(12): e00490-2). Most recently, inhibition of STING pathway was reported to reduce servere lung inflammation in the late phase of SARS-CoV-2 infection (Domizio D et al. Nature, 2022, 603, 145-151), highlighting that the contribution of STING modulators to face the challenge of future virus-related outbreaks.

The STING signaling pathway is shown to be essential for endogenous antitumor T cell responses as well as radiation-induced antitumor T cell responses (Woo S R, et al. Immunity. 2014; 41: 830-842; Deng L, et al. Immunity. 2014; 41: 843-852). STING-deficient mice were highly susceptible to tumor development, diminished antitumor T cell immunity, and impaired responses to immunotherapy (Deng L et al. Immunity. 2014; 41: 843-852).

STING-cGAS signaling is indispensable for the antitumor effect of immune-check point blockade. The antitumor ability of immune checkpoint inhibitors was abrogated in cGAS-deficient mice, indicating the importance of STING in the therapeutic efficacy of immune checkpoint inhibitors (Wang H et al. Proc Natl Acad Sci USA. 2017; 114: 1637-1642). Indeed, STING agonists were shown to be effective against tumors resistant to programmed cell death protein 1 (PD1) blockade (Fu J et al. Sci. Transl Med. 2015, 7, 283ra52). A combination STING agonist ADU-S100 with a PD-L1 inhibitor and an OX40R agonist led to the effective activation of innate immunity to support T cell priming and reduce tumor growth in mouse model of HER2+breast tumors (Foote J B et al. Cancer Immunol Res. 2017; 5: 468-479). Taken together, STING activators may play an important role in fighting cancer.

Initial STING agonist small molecules were synthesized as the CDN natural ligand derivatives. However, because of poor stability properties, CDN-based agonist administration is limited to intratumoral delivery in the pre-clinical studies (Corrales L. et al. Cell Rep. 2015, 11, 1108-1030; Sivick K. E et al. Cell Rep. 2019, 29, 785-789) and with mixed success in humans. As such, a systemic STING-activating agent has considerable potential utility as a therapeutic for cancer and infectious diseases. A non-CDN agonist 5,6-dimethylxanthenone-4-acetic acid (DMXAA), the best-characterized STING agonist, was investigated as an experimental anticancer immunomodulator and generated fundamental knowledge (Baguley B C et al. Bio Drugs. 1997; 8: 119-127). However, DMXAA specifically binds to murine STING, which impacts successful clinical translation in human cancer patients, highlighting the importance of species selectivity in drug development for human diseases (Roberts Z J et al. J Exp Med. 2007, 204: 1559-1569). A number of clinical trials of STING modulators were prematurely terminated in cancer patients due to poor preliminary efficacy or increased rate of adverse reactions (www.clincaltrials.gov).

There is an ongoing need for novel STING modulators and treatment methods for cancers and infectious diseases as well as other STING dependent diseases and disorders to provide patients with improved clinical effectiveness and reduced side effects.

### SUMMARY OF THE INVENTION

The invention is based in part on novel modulators of STING, pharmaceutical compositions thereof and methods of their preparation and use in treating or reducing various diseases or disorders. In particular, compounds, compositions and methods of the invention are useful in treating diseases or disorders mediated by or associated with STING.

In one aspect, the invention generally relates to a compound having the structural formula I or formula II:

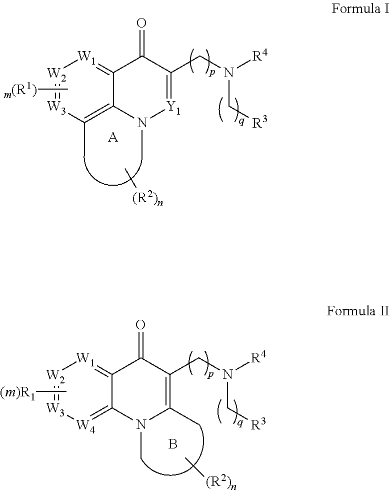

wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
   or a pharmaceutically acceptable form or an isotope derivative thereof.

In another aspect, the invention generally relates to a compound having the structural formula I or formula II:

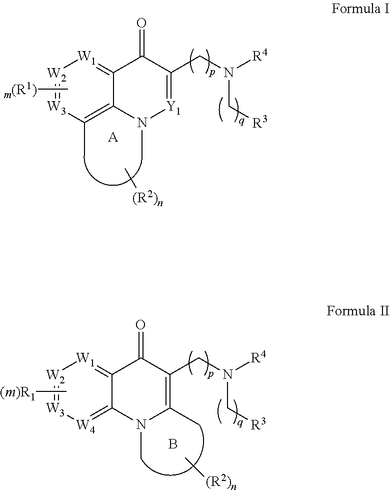

wherein
Ring A is a 5-, 6- or 7-membered substituted or unsubstituted heterocycle;
Ring B is a 4-, 5- or 6-membered substituted or unsubstituted heterocycle;
Y₁ is CH, CMe or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, NRR′, N(R)C(═O)R, N(R)C(═O)(O)R, OC(═O)NRR′, C(═O)R, C(═O)NRR′, N(R)S(O)₂R, S(O)₂R and S(O)₂NRR′; wherein R¹ is optionally substituted with 1 to 4 same or different R^{a} or R^{b};
R² is selected from the group consisting of halo, oxo, OH, CN, OR, CF₃ and C₁₋₆ alkyl;
R³ is a 5- to 6-membered aromatic carbocyclic or heterocyclic ring, or a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, N(R)C(═O)R, or C(═O)R, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, -5- to 6-membered aromatic carbocyclic or heterocyclic ring, C₁₋₂ alkyl-carbocyclyl, or C₁₋₂ alkyl-heterocyclyl, wherein the aforementioned aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n is 0, 1, 2 or 3;
p is 1, 2 or 3; and
q is 1, 2 or 3;
or a pharmaceutically acceptable form or an isotope derivative thereof.

In yet another aspect, the invention generally relates to a compound having the structural formula III:

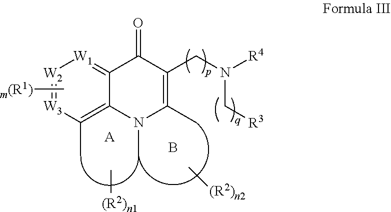

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
or a pharmaceutically acceptable form or an isotope derivative thereof.

In yet another aspect, the invention generally relates to a compound having the structural formula III:

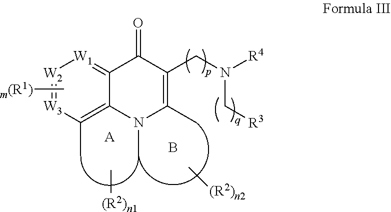

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein R¹ is optionally substituted with 1 to 4 same or different R^{a} or R^{b}.
R² is selected from the group consisting of halo, oxo, OH, CN, OR, CF₃ and C₁₋₆ alkyl;
R³ is a 5- to 6-membered aromatic carbocyclic or heterocyclic ring, or a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, halo, OH, OC₁₋₃ alkyl, and CN;
R^{a} is OH, NRR′, halogen, CN, NO₂, C₁₋₂ alkyl, C₁₋₂ haloalkyl, C₁₋₄ alkyl-NRR′, C₁₋₄ alkyl-OR, C₁₋₂ alkoxy, C(═O)NRR′, N(R)C(═O)R, or C(═O)R, or two R^{a}'s form ═O, or two R^{a}'s together with the carbon atom to which they are bonded form a 3- to 5-membered saturated or unsaturated carbocyclic or heterocyclic ring;
R is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, -5- to 6-membered aromatic carbocyclic or heterocyclic ring, C₁₋₂ alkyl-carbocyclyl, or C₁₋₂ alkyl-heterocyclyl, wherein the aforementioned aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
   or a pharmaceutically acceptable form or an isotope derivative thereof.
   In yet another aspect, the invention generally relates to a pharmaceutical composition comprising a compound of formula I, II or III, or a compound disclosed herein, effective to treat or reduce one or more diseases or disorders, in a mammal, including a human.

In yet another aspect, the invention generally relates to a unit dosage form comprising a pharmaceutical composition disclosed herein.

In yet another aspect, the invention generally relates to a method for treating or reducing a disease or disorder mediated by or associated with STING, comprising administering to a subject in need thereof a pharmaceutical composition comprising a compound having the structural formula I or formula II.

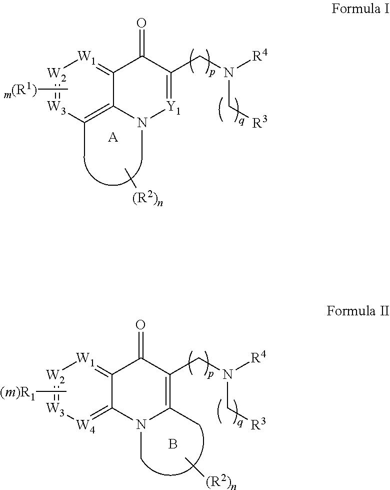

wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein R¹ is optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
   or a pharmaceutically acceptable form or an isotope derivative thereof.

In yet another aspect, the invention generally relates to a method for treating or reducing a disease or disorder mediated by or associated with STING, comprising administering to a subject in need thereof a pharmaceutical composition comprising a compound having the structural formula III:

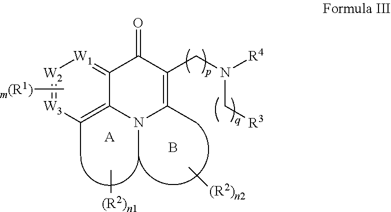

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
   or a pharmaceutically acceptable form or an isotope derivative thereof.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “administration” of a disclosed compound encompasses the delivery to a subject of a compound as described herein, or a prodrug or other pharmaceutically acceptable form thereof, using any suitable formulation or route of administration, as discussed herein.

The terms “disease”, “disorder” and “condition” are used interchangeably unless indicated otherwise.

As used herein, the terms “effective amount” or “therapeutically effective amount” refer to that amount of a compound or pharmaceutical composition described herein that is sufficient to effect the intended application including, but not limited to, disease treatment, as illustrated below.

In some embodiments, the amount is that effective for detectable killing or inhibition of the growth or spread of cancer cells; the size or number of tumors; or other measure of the level, stage, progression or severity of the cancer.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, CN, —COOH, —CH₂CN, —O—C₁-C₆ alkyl, C₁-C₆ alkyl, —OC₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —NHC(O)C₁-C₆ alkyl, —C(O)NHC₁-C₆ alkyl, —S(O)₂—C₁-C₆ alkyl, —S(O)NHC₁-C₆ alkyl, and S(O)N(C₁-C₆ alkyl)₂.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

As used herein, the term an “isolated” or “substantially isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “purified” when used in reference to a molecule, it means that the concentration of the molecule being purified has been increased relative to molecules associated with it in its natural environment, or environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

Definitions of specific functional groups and chemical terms are described in more detail below. When a range of values is listed, it is intended to encompass each value and sub-range within the range. For example, “C₁₋₄ alkyl” is intended to encompass, C₁, C₂, C₃, C₄, C₁₋₃, C₁₋₂, C₂₋₄, C₃₋₄ and C₂₋₃ alkyl groups.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

Unless otherwise specifically defined, the term “aromatic” or “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, H, halogen, —O—C₁-C₆ alkyl, C₁-C₆ alkyl, —C₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆alkyl, NH₂, NH(C₁-C₆ alkyl), N(C₁-C₆ alkyl)₂, —S(O)₂—C₁-C₆ alkyl, —S(O)NHC₁-C₆alkyl, and S(O)N(C₁-C₆ alkyl)₂. The substituents can themselves be optionally substituted. Furthermore, when containing two fused rings the aryl groups herein defined may have an unsaturated or partially saturated ring fused with a fully unsaturated ring. Exemplary ring systems of these aryl groups include indanyl, indenyl, tetrahydronaphthalenyl, and tetrahydrobenzoannulenyl.

The term “halogen” or “halo” refers to fluorine (F), chlorine (Cl), bromine (Br) and iodine (I).

Unless otherwise specifically defined, the terms “heteroaryl” or “hetero-aromatic” as used herein, means a monocyclic heteroaryl ring or a bicyclic heteroaryl ring. The monocyclic heteroaryl ring is a 5- or 6-membered ring. The 5-membered ring has two double bonds and contains one, two, three or four heteroatoms independently selected from the group consisting of N, O, and S. The 6-membered ring has three double bonds and contains one, two, three or four heteroatoms independently selected from the group consisting of N, O, and S. The bicyclic heteroaryl ring consists of the 5- or 6-membered heteroaryl ring fused to a phenyl group or the 5- or 6-membered heteroaryl ring fused to a cycloalkyl group or the 5- or 6-membered heteroaryl ring fused to a cycloalkenyl group or the 5- or 6-membered heteroaryl ring fused to another 5- or 6-membered heteroaryl ring. Nitrogen heteroatoms contained within the heteroaryl may be optionally oxidized to the N-oxide or optionally protected with a nitrogen protecting group known to those of skill in the art. The heteroaryl is connected to the parent molecular moiety through any carbon atom or any nitrogen atom contained within the heteroaryl. Representative examples of heteroaryl include, but are not limited to, benzothienyl, benzoxadiazolyl, cirmolinyl, 5,6-dihydroisoquinolinyl, 7,8-dihydroisoquinolinyl, 5,6-dihydroquinolinyl, 7,8-dihydroquinolinyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, pyridinium N-oxide, quinolinyl, 5,6,7,8-tetrahydroisoquinolinyl, 5,6,7,8-tetrahydroquinolinyl, tetrazolyl, thiadiazolyl, thiazolyl, thienopyridinyl, thienyl, triazolyl, and triazinyl.

The terms “heteroaryl” or “hetero-aromatic” groups of the present invention are substituted with 0, 1, 2, 3, or 4 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂, and (NZ₁Z₂)carbonyl. The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, and formyl. Representative examples of NZ₁Z₂ include, but are not limited to, amino, methylamino, acetylamino, and acetylmethylamino.

As used herein, the term “alkoxy” refers to an —O-alkyl radical.

As used herein, the terms “cycloalkyl” and “carbocyclyl” each refers to a monocyclic or polycyclic radical that contains only carbon and hydrogen, and can be saturated or partially unsaturated. Unless stated otherwise in the specification, the term is intended to include both substituted and unsubstituted cycloalkyl groups. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “heterocycloalkyl” refers to a cycloalkyl radical, which have one or more skeletal chain atoms selected from an atom other than carbon, e.g., O, N, S, P or combinations thereof. Unless stated otherwise in the specification, the term is intended to include both substituted and unsubstituted heterocycloalkyl groups. Illustrative examples of heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, 1-cyclopropyl-4-methyl-piperazin-2-yl. 2-pyrrolinyl, 3-pyrrolinyl, dihydro-2H-pyranyl, 1,2,3,4-tetrahydropyridine, 3,4-dihydro-2H-[1,4]oxazine, etc.

As used herein, the terms “heterocycle”, “heterocyclic” or “heterocyclo” refer to fully saturated or partially unsaturated cyclic groups, for example, 3 to 7 membered monocyclic, 7 to 12 membered bicyclic, or 10 to 15 membered tricyclic ring systems, which have at least one heteroatom in at least one ring, wherein 0, 1, 2 or 3 atoms of each ring may be substituted by a substituent. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

### DETAILED DESCRIPTION OF THE INVENTION

The invention is based in part on the discovery of novel modulators of STING, pharmaceutical compositions thereof and methods of their preparation and use in therapy of various diseases and conditions, such as cancers (e.g., breast, colon, endometrial, kidney, lung, melanoma, prostate, thyroid cancer and leukemia).

In one aspect, the invention generally relates to a compound the structural formula I or formula II:

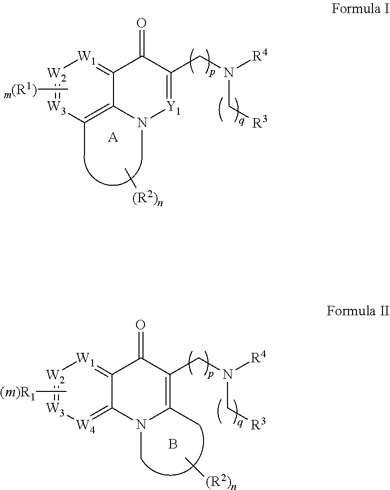

wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
   or a pharmaceutically acceptable form or an isotope derivative thereof.

In certain embodiments, the compound has the formula I.

In certain embodiments, Ring A is substituted or unsubstituted 5- to 7-membered heterocycle.

In certain embodiments, Ring A is:

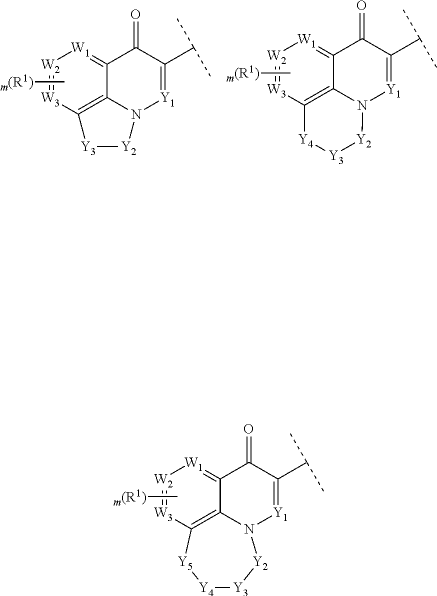

wherein:
Y₂ is CRR′ or C═O; and
each of Y₃, Y₄ and Y₅ is independently selected from the group consisting of N, O, CRR′ and C═O.

Non-limiting examples of include:

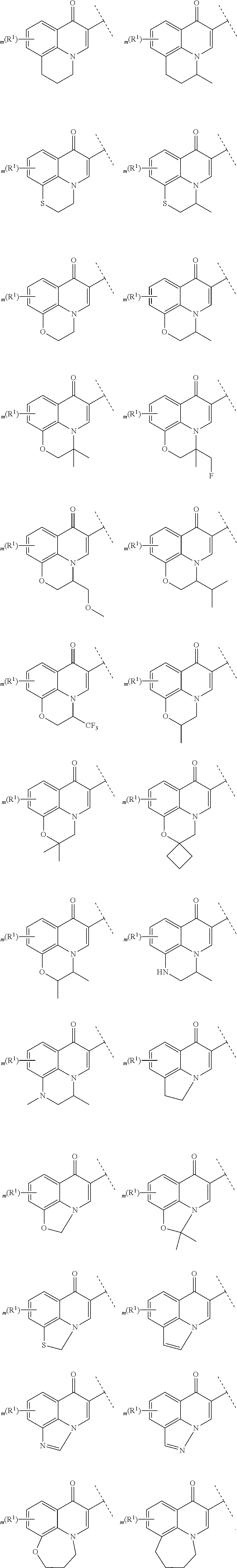

In certain embodiments of Formula I, W₁ is CH or C-halo (e.g., CF), W₂ is CH or C-halo (e.g., CF), and W₃ is CR¹.

In certain embodiments of Formula I, W₁ is N, W₂ is CH or C-halo (e.g., CF), and W₃ is CR¹.

In certain embodiments of Formula I, p is 1 and q is 1.

In certain embodiments, the compound has the formula II.

In certain embodiments, Ring B is aromatic. In certain embodiments, Ring B is not aromatic.

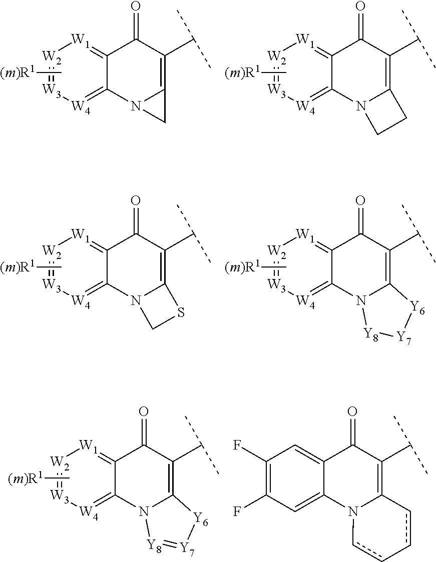

### Y₆ is CRR′ or S, O; and

each of Y₇ and Y₈ is independently selected from the group consisting of N, O, and CRR′.

Non-limiting examples of include:

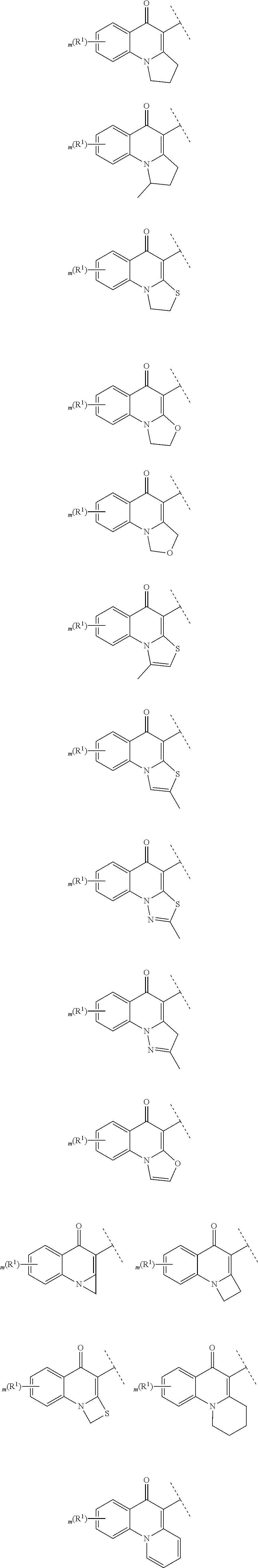

In certain embodiments of Formula II, W₁ is CH or C-halo (e.g., CF), W₂ is CH or C-halo (e.g., CF), W₃ is CR¹, and W₄ is CH or C-halo (e.g., CF).

In certain embodiments of Formula II, W₁ is N, W₂ is CH or C-halo (e.g., CF), W₃ is CR¹, and W₄ is CH or C-halo (e.g., CF).

In certain embodiments of Formula II, p is 1 and q is 1.

In another aspect, the invention generally relates to a compound having the structural formula III:

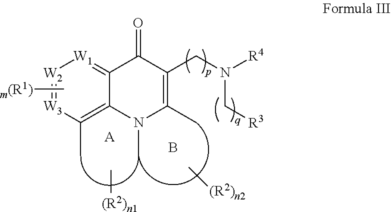

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
   or a pharmaceutically acceptable form or an isotope derivative thereof.

In certain embodiments of Formula III, W₁ is CH or C-halo (e.g., CF), W₂ is CH or C-halo (e.g., CF), and W₃ is CR¹.

In certain embodiments of Formula III, W₁ is N, W₂ is CH or C-halo (e.g., CF), and W₃ is CR¹.

In certain embodiments of Formula III, p is 1 and q is 1.

Non-limiting examples of Rings A-B include:

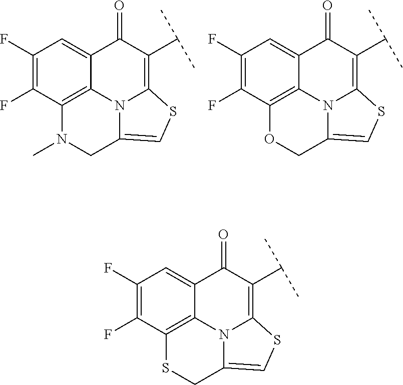

In certain embodiments of Formula I, II or III, R³ is a 5- or 6-membered substituted or unsubstituted aromatic or heteroaromatic group;

Non-limiting examples of include:

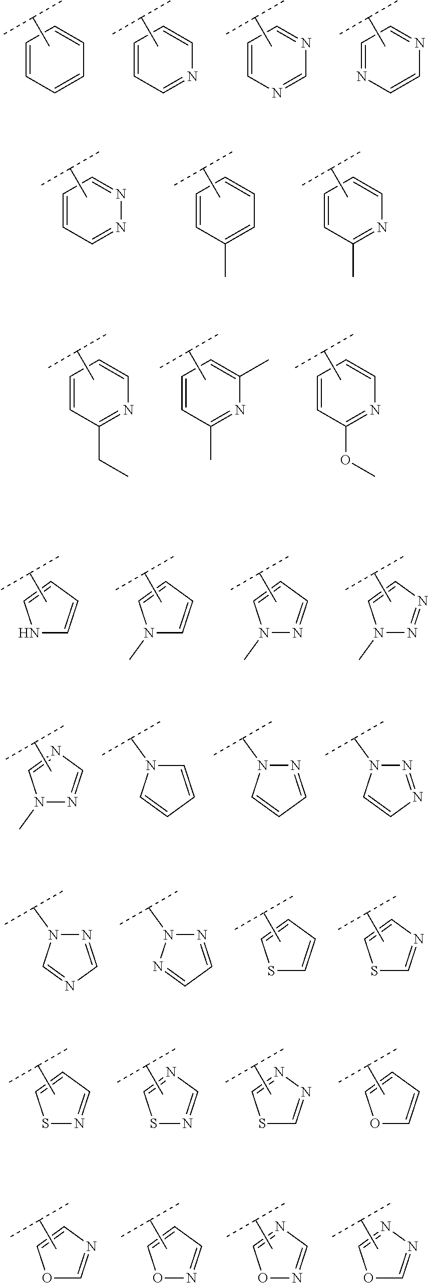

In certain embodiments of formula I, II or III, R⁴ a 5- or 6-membered substituted or unsubstituted carbocycle or heterocycle;

Non-limiting examples of include:

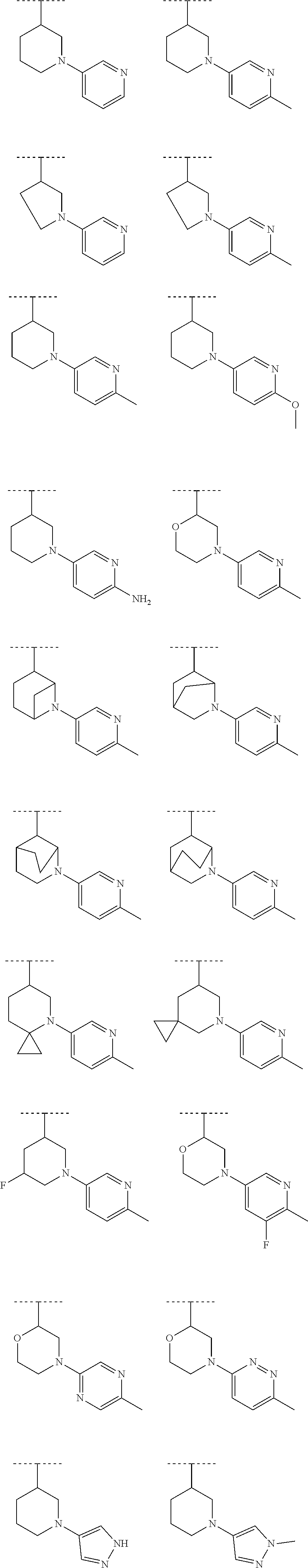

In certain embodiments of formula I, R² is substituted or unsubstituted aromatic or heteroaromatic;

Non-limiting examples of include:

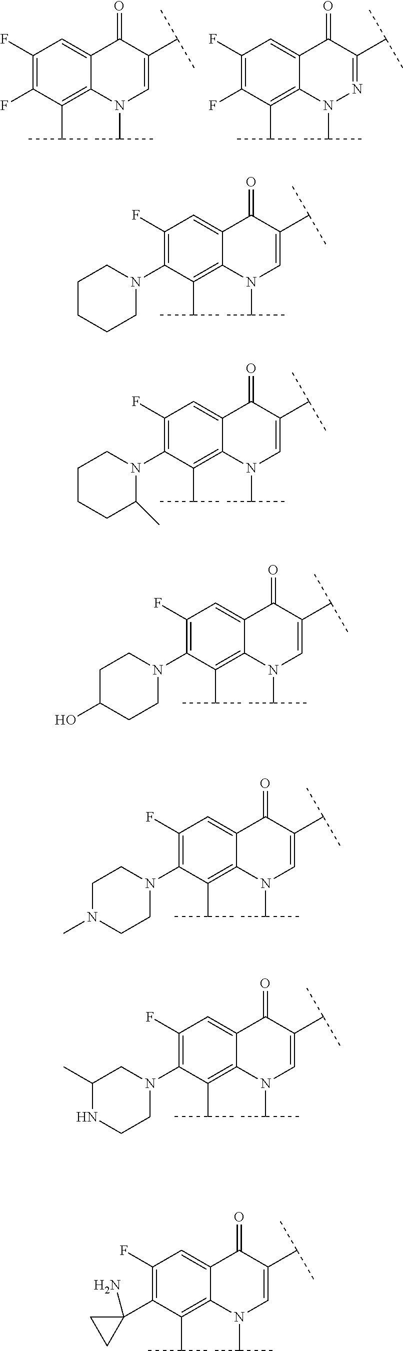

In certain embodiments of formula II, R² substituted or unsubstituted aromatic or heteroaromatic;

Non-limiting examples of include:

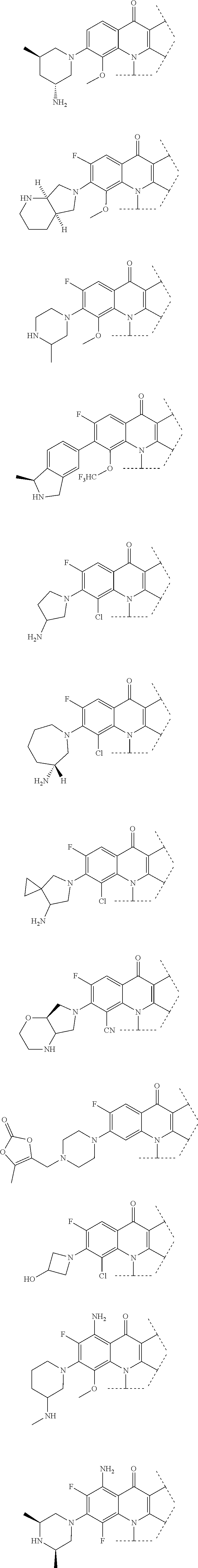

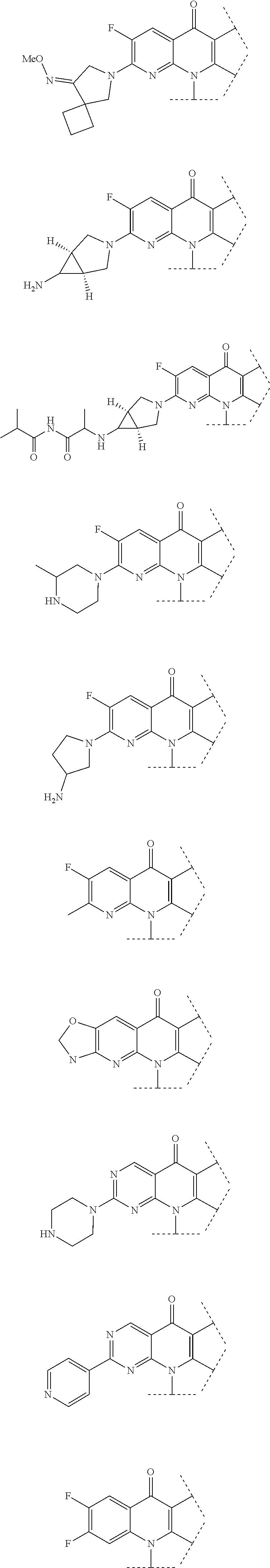

Exemplary compounds of the invention include but not limited to:

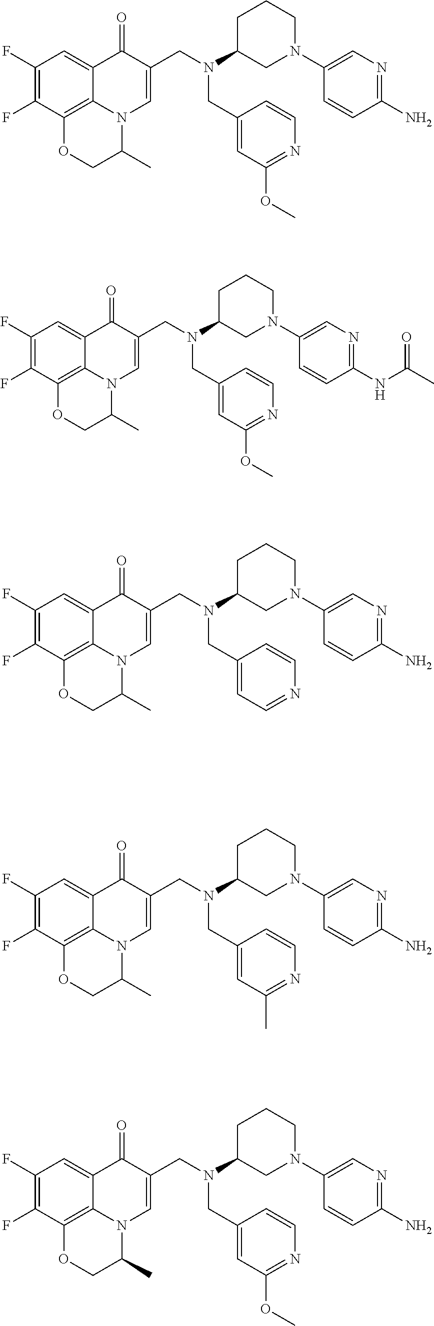 [SMILES: COc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3C)[C@H]2CCCN(c3ccc(NC(C)=O)nc3)C2)ccn1 ; CC1COc2c(F)c(F)cc3c(=O)c(CN(Cc4ccncc4)[C@H]4CCCN(c5ccc(N)nc5)C4)cn1c23 ; Cc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OC[C@@H]3C)[C@H]2CCCN(c3ccc(N)nc3)C2)ccn1]

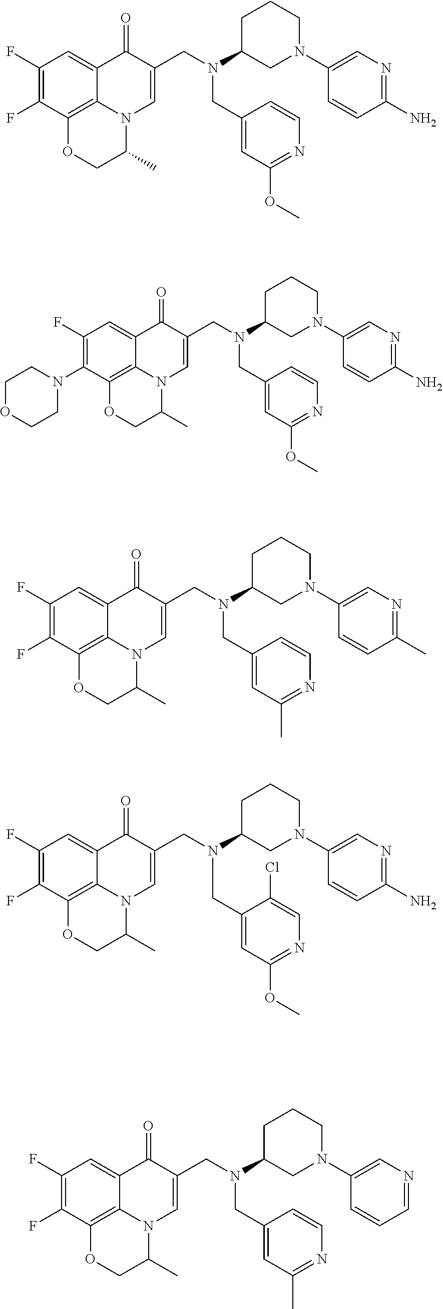 [SMILES: COc1cc(CN(Cc2cn3c4c(c(F)c(F)cc4c2=O)OC[C@H]3C)[C@H]2CCCN(c3ccc(N)nc3)C2)ccn1 ; COc1cc(CN(Cc2cn3c4c(c(N5CCOCC5)c(F)cc4c2=O)OCC3C)[C@H]2CCCN(c3ccc(N)nc3)C2)ccn1 ; Cc1ccc(N2CCC[C@H](N(Cc3ccnc(C)c3)Cc3cn4c5c(c(F)c(F)cc5c3=O)OCC4C)C2)cn1 ; COc1cc(CN(Cc2cn3c4c(c(F)c(F)cc4c2=O)OCC3C)[C@H]2CCCN(c3ccc(N)nc3)C2)c(Cl)cn1 ; Cc1cc(CN(Cc2cn3c4c(c(F)c(F)cc4c2=O)OCC3C)[C@H]2CCCN(c3cccnc3)C2)ccn1]

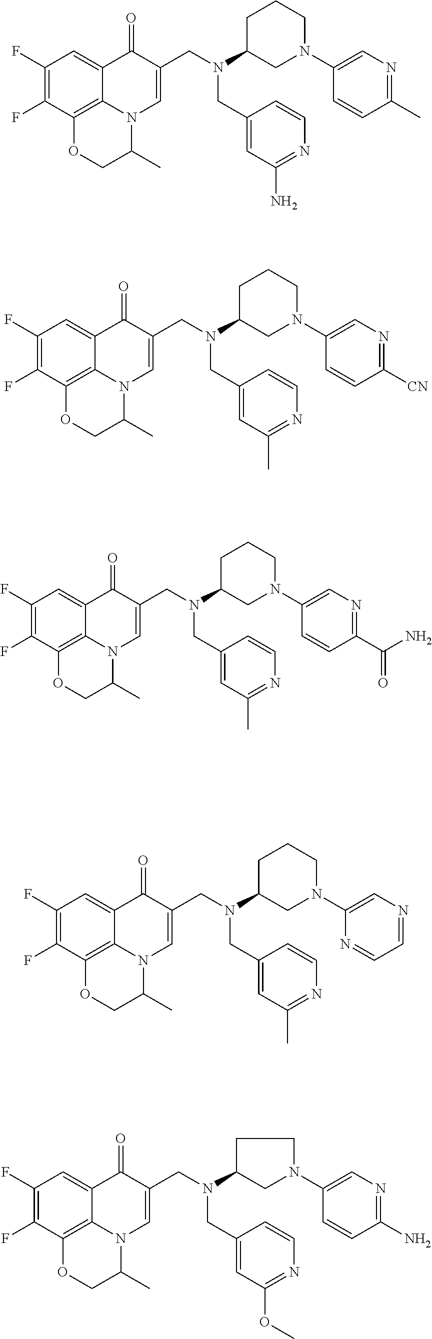 [SMILES: Cc1ccc(N2CCC[C@H](N(Cc3ccnc(N)c3)Cc3cn4c5c(c(F)c(F)cc5c3=O)OCC4C)C2)cn1 ; Cc1cc(CN(Cc2cn3c4c(c(F)c(F)cc4c2=O)OCC3C)[C@H]2CCCN(c3ccc(C#N)nc3)C2)ccn1 ; Cc1cc(CN(Cc2cn3c4c(c(F)c(F)cc4c2=O)OCC3C)[C@H]2CCCN(c3ccc(C(N)=O)nc3)C2)ccn1 ; Cc1cc(CN(Cc2cn3c4c(c(F)c(F)cc4c2=O)OCC3C)[C@H]2CCCN(c3cnccn3)C2)ccn1 ; COc1cc(CN(Cc2cn3c4c(c(F)c(F)cc4c2=O)OCC3C)[C@H]2CCN(c3ccc(N)nc3)C2)ccn1]

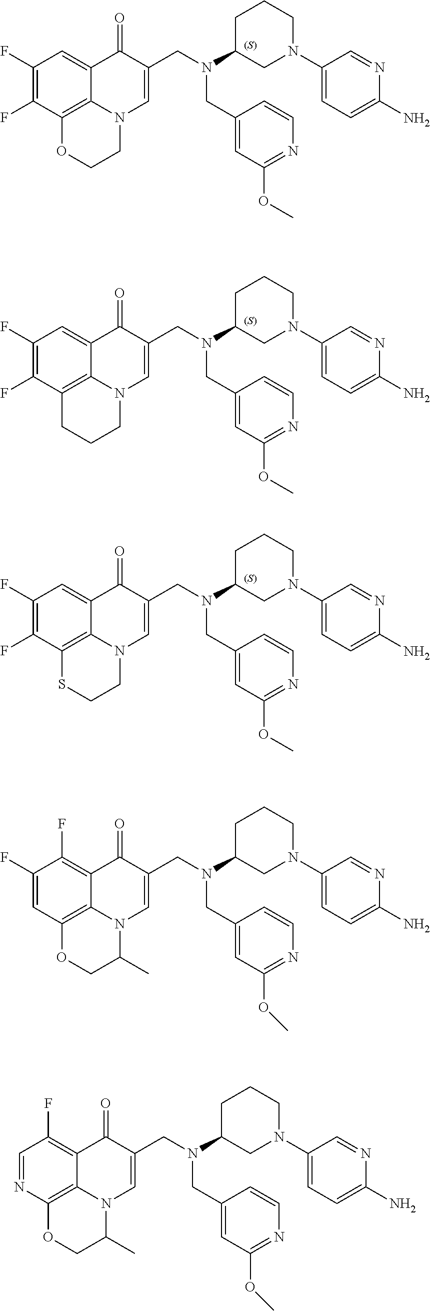

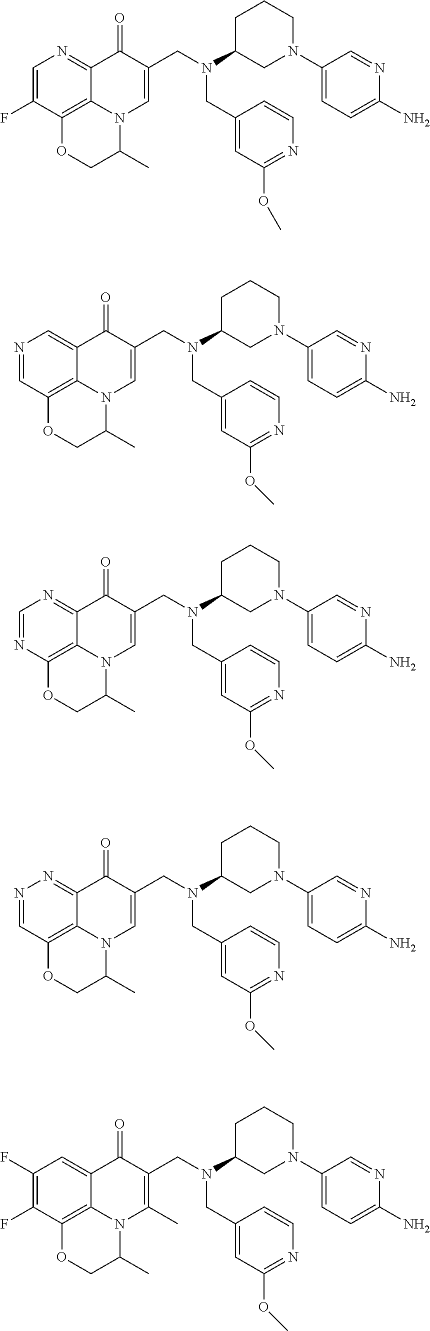 [SMILES: COc1cc(CN(Cc2cn3c4c(c(F)cnc4c2=O)OCC3C)[C@H]2CCCN(c3ccc(N)nc3)C2)ccn1 ; COc1cc(CN(Cc2cn3c4c(cncc4c2=O)OCC3C)[C@H]2CCCN(c3ccc(N)nc3)C2)ccn1 ; COc1cc(CN(Cc2cn3c4c(ncnc4c2=O)OCC3C)[C@H]2CCCN(c3ccc(N)nc3)C2)ccn1 ; COc1cc(CN(Cc2cn3c4c(cnnc4c2=O)OCC3C)[C@H]2CCCN(c3ccc(N)nc3)C2)ccn1 ; COc1cc(CN(Cc2c(C)n3c4c(c(F)c(F)cc4c2=O)OCC3C)[C@H]2CCCN(c3ccc(N)nc3)C2)ccn1]

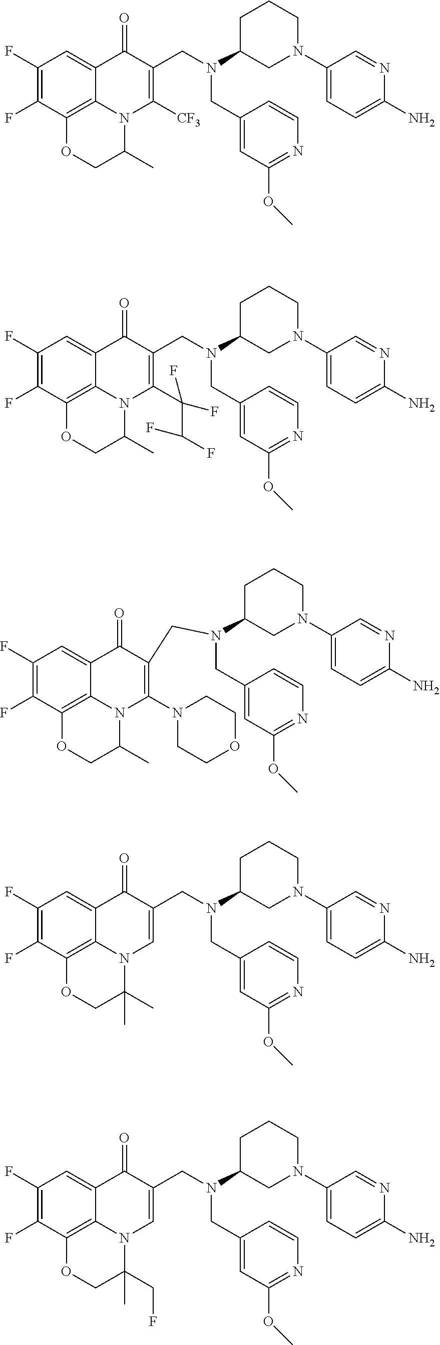 [SMILES: COc1cc(CN(Cc2c(C(F)(F)F)n3c4c(c(F)c(F)cc4c2=O)OCC3C)[C@H]2CCCN(c3ccc(N)nc3)C2)ccn1 ; COc1cc(CN(Cc2c(C(F)(F)C(F)F)n3c4c(c(F)c(F)cc4c2=O)OCC3C)[C@H]2CCCN(c3ccc(N)nc3)C2)ccn1 ; COc1cc(CN(Cc2c(N3CCOCC3)n3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3(C)C)[C@H]2CCCN(c3ccc(N)nc3)C2)ccn1 ; COc1cc(CN(Cc2cn3c4c(c(F)c(F)cc4c2=O)OCC3(C)CF)[C@H]2CCCN(c3ccc(N)nc3)C2)ccn1]

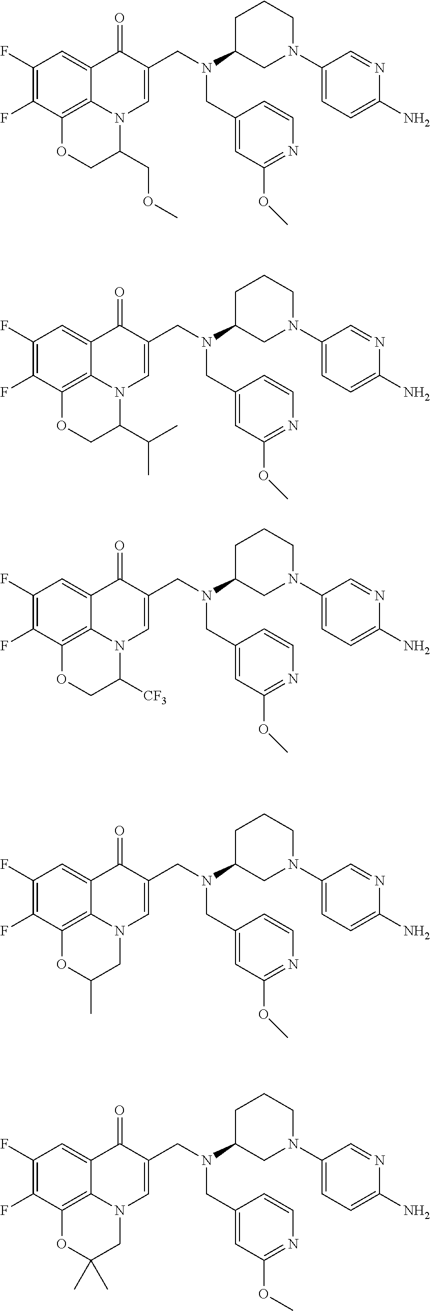

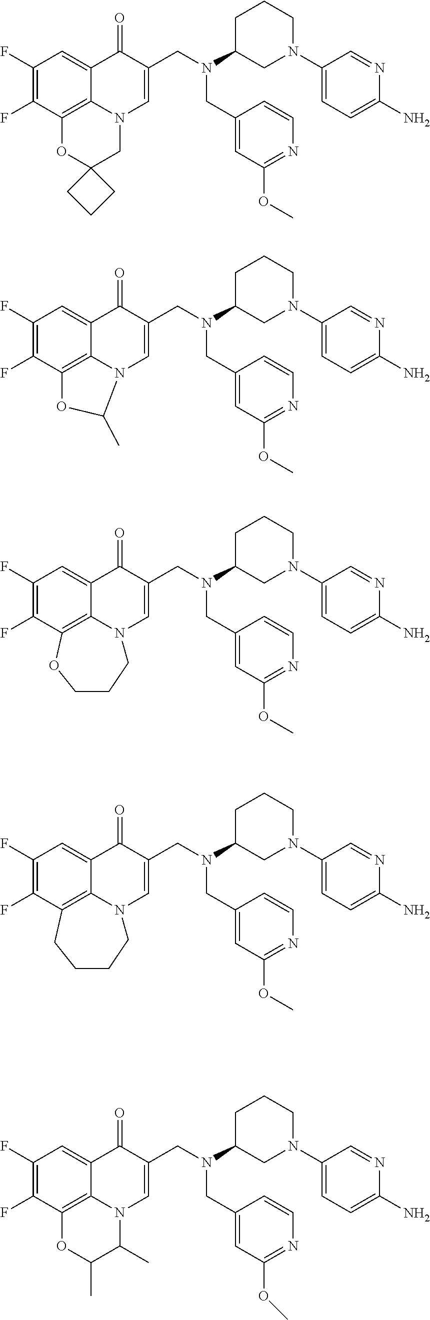 [SMILES: COc1cc(CN(Cc2cn3c4c(c(F)c(F)cc4c2=O)OC2(CCC2)C3)[C@H]2CCCN(c3ccc(N)nc3)C2)ccn1 ; COc1cc(CN(Cc2cn3c4c(c(F)c(F)cc4c2=O)OC3C)[C@H]2CCCN(c3ccc(N)nc3)C2)ccn1 ; COc1cc(CN(Cc2cn3c4c(c(F)c(F)cc4c2=O)OCCC3)[C@H]2CCCN(c3ccc(N)nc3)C2)ccn1 ; COc1cc(CN(Cc2cn3c4c(c(F)c(F)cc4c2=O)CCCC3)[C@H]2CCCN(c3ccc(N)nc3)C2)ccn1 ; COc1cc(CN(Cc2cn3c4c(c(F)c(F)cc4c2=O)OC(C)C3C)[C@H]2CCCN(c3ccc(N)nc3)C2)ccn1]

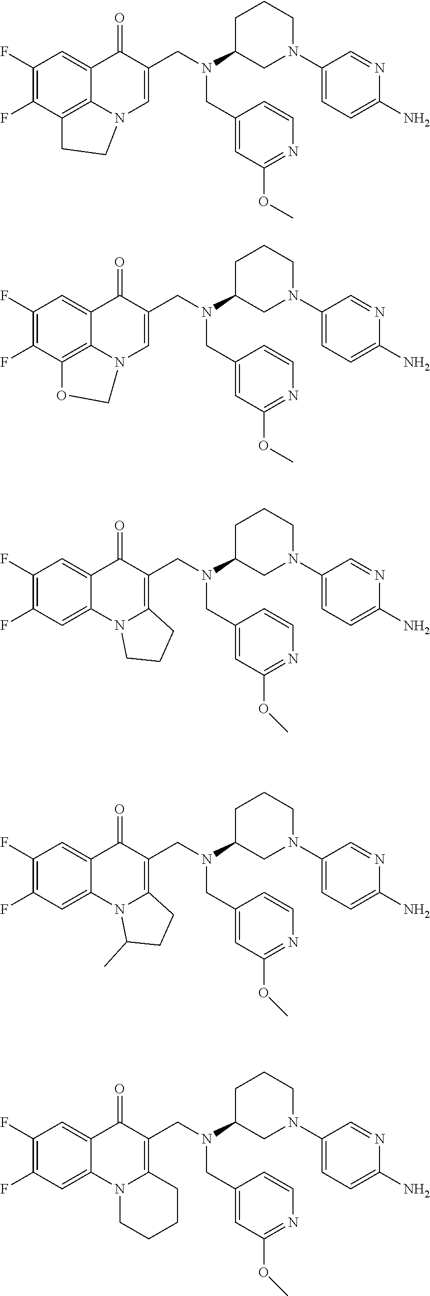

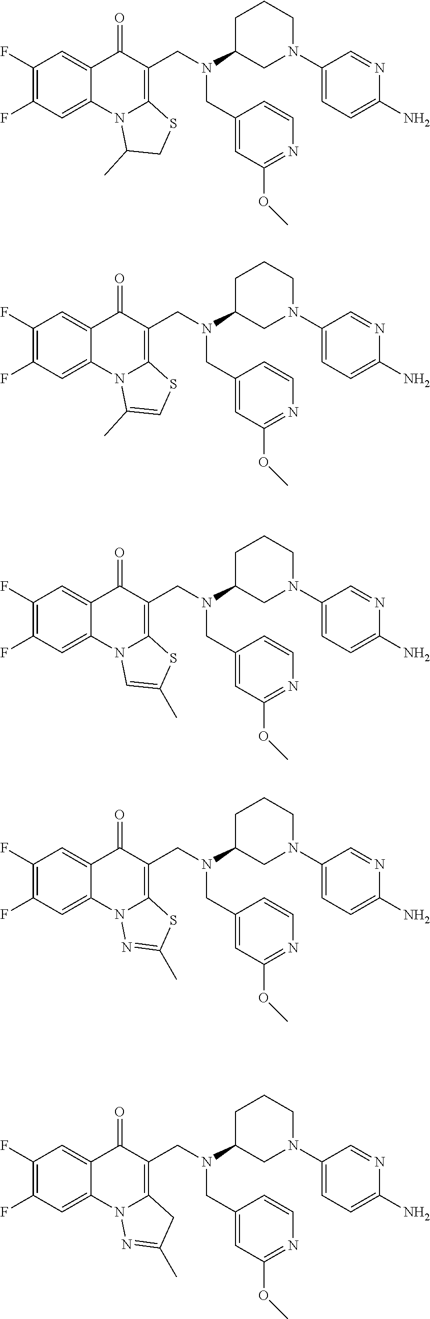

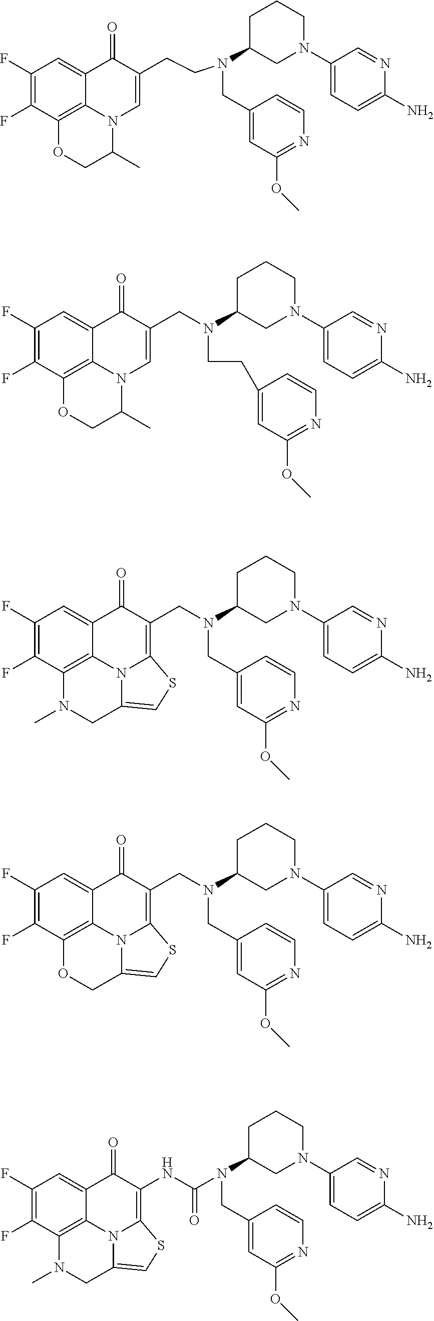 [SMILES: COc1cc(CN(CCc2cn3c4c(c(F)c(F)cc4c2=O)OCC3C)[C@H]2CCCN(c3ccc(N)nc3)C2)ccn1 ; COc1cc(CCN(Cc2cn3c4c(c(F)c(F)cc4c2=O)OCC3C)[C@H]2CCCN(c3ccc(N)nc3)C2)ccn1 ; COc1cc(CN(Cc2c(=O)c3cc(F)c(F)c4c3n3c(csc23)CN4C)[C@H]2CCCN(c3ccc(N)nc3)C2)ccn1 ; COc1cc(CN(Cc2c(=O)c3cc(F)c(F)c4c3n3c(csc23)CO4)[C@H]2CCCN(c3ccc(N)nc3)C2)ccn1 ; COc1cc(CN(C(=O)Nc2c(=O)c3cc(F)c(F)c4c3n3c(csc23)CN4C)[C@H]2CCCN(c3ccc(N)nc3)C2)ccn1]

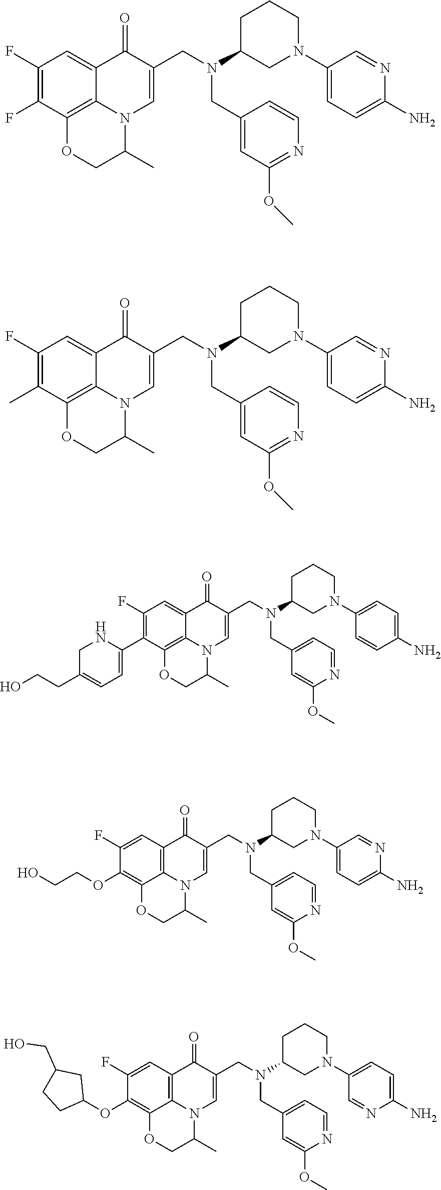 [SMILES: COc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(C)c(F)cc4c2=O)OCC3C)[C@H]2CCCN(c3ccc(N)nc3)C2)ccn1 ; COc1cc(CN(Cc2cn3c4c(c(C5=CC=C(CCO)CN5)c(F)cc4c2=O)OCC3C)[C@H]2CCCN(c3ccc(N)cc3)C2)ccn1 ; COc1cc(CN(Cc2cn3c4c(c(OCCO)c(F)cc4c2=O)OCC3C)[C@H]2CCCN(c3ccc(N)nc3)C2)ccn1 ; COc1cc(CN(Cc2cn3c4c(c(OC5CCC(CO)C5)c(F)cc4c2=O)OCC3C)[C@@H]2CCCN(c3ccc(N)nc3)C2)ccn1]

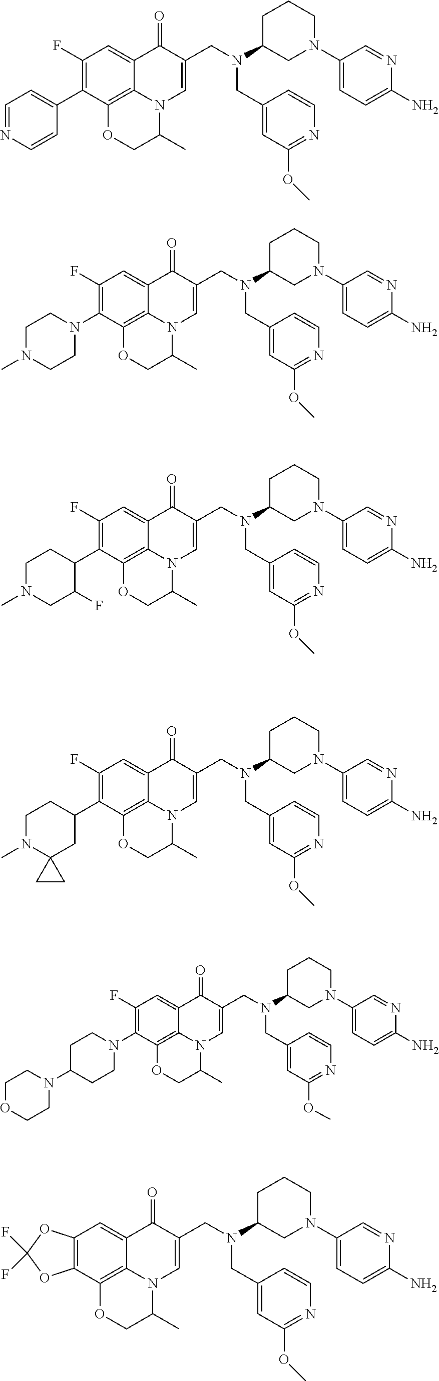

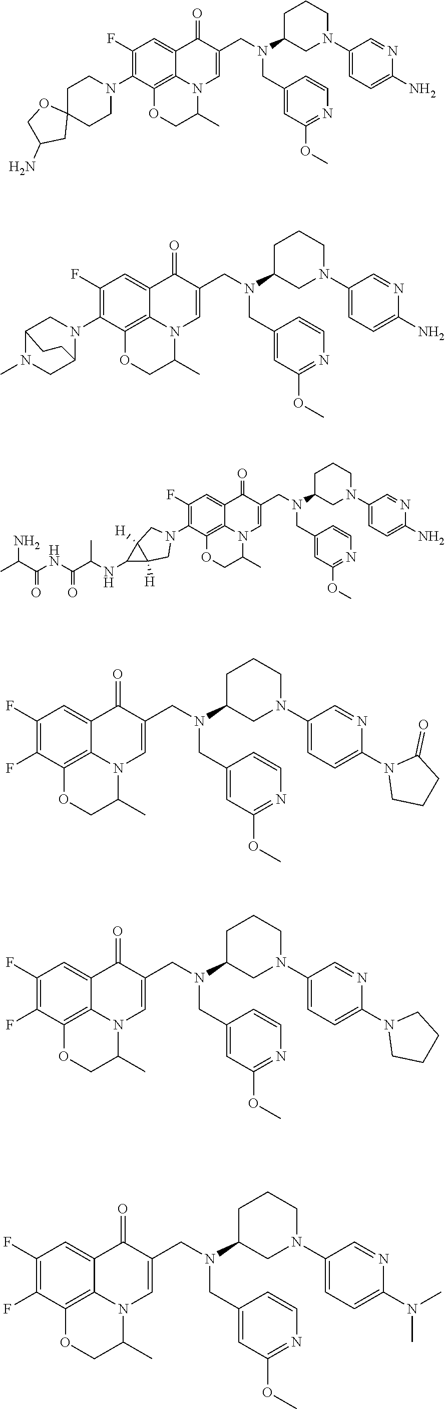

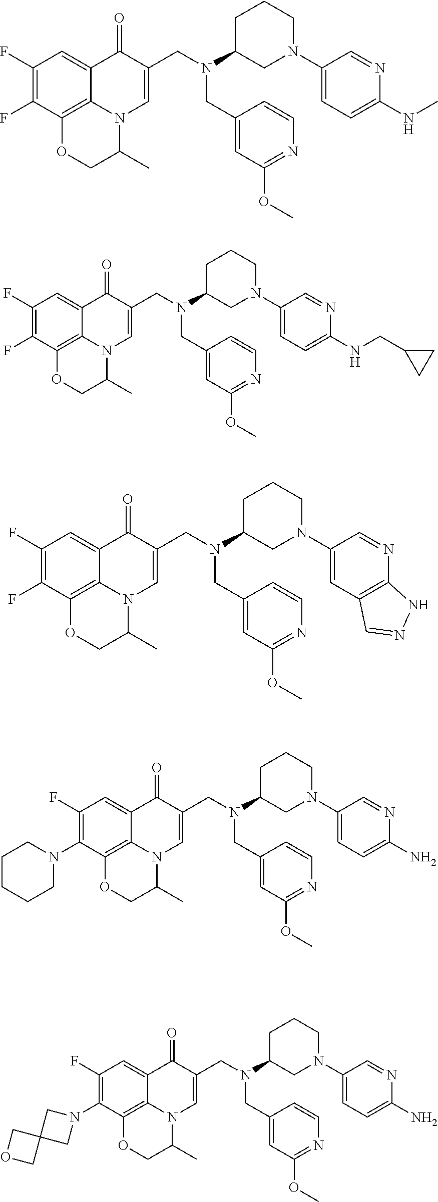 [SMILES: CNc1ccc(N2CCC[C@H](N(Cc3ccnc(OC)c3)Cc3cn4c5c(c(F)c(F)cc5c3=O)OCC4C)C2)cn1 ; COc1cc(CN(Cc2cn3c4c(c(F)c(F)cc4c2=O)OCC3C)[C@H]2CCCN(c3ccc(NCC4CC4)nc3)C2)ccn1 ; COc1cc(CN(Cc2cn3c4c(c(F)c(F)cc4c2=O)OCC3C)[C@H]2CCCN(c3cnc4[nH]ncc4c3)C2)ccn1 ; COc1cc(CN(Cc2cn3c4c(c(N5CCCCC5)c(F)cc4c2=O)OCC3C)[C@H]2CCCN(c3ccc(N)nc3)C2)ccn1 ; COc1cc(CN(Cc2cn3c4c(c(N5CC6(COC6)C5)c(F)cc4c2=O)OCC3C)[C@H]2CCCN(c3ccc(N)nc3)C2)ccn1]

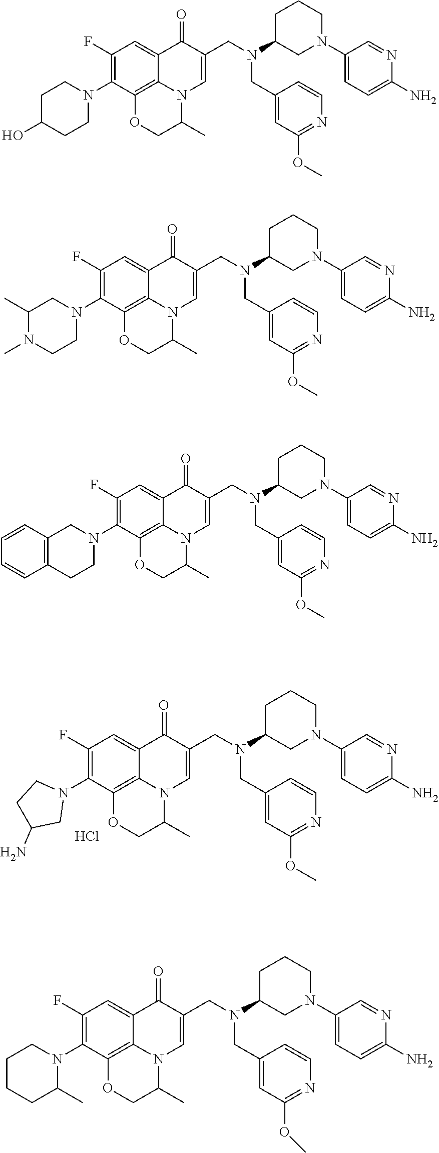 [SMILES: COc1cc(CN(Cc2cn3c4c(c(N5CCC(O)CC5)c(F)cc4c2=O)OCC3C)[C@H]2CCCN(c3ccc(N)nc3)C2)ccn1 ; COc1cc(CN(Cc2cn3c4c(c(N5CCN(C)C(C)C5)c(F)cc4c2=O)OCC3C)[C@H]2CCCN(c3ccc(N)nc3)C2)ccn1 ; COc1cc(CN(Cc2cn3c4c(c(N5CCc6ccccc6C5)c(F)cc4c2=O)OCC3C)[C@H]2CCCN(c3ccc(N)nc3)C2)ccn1 ; COc1cc(CN(Cc2cn3c4c(c(N5CCC(N)C5)c(F)cc4c2=O)OCC3C)[C@H]2CCCN(c3ccc(N)nc3)C2)ccn1 ; Cl ; COc1cc(CN(Cc2cn3c4c(c(N5CCCCC5C)c(F)cc4c2=O)OCC3C)[C@H]2CCCN(c3ccc(N)nc3)C2)ccn1]

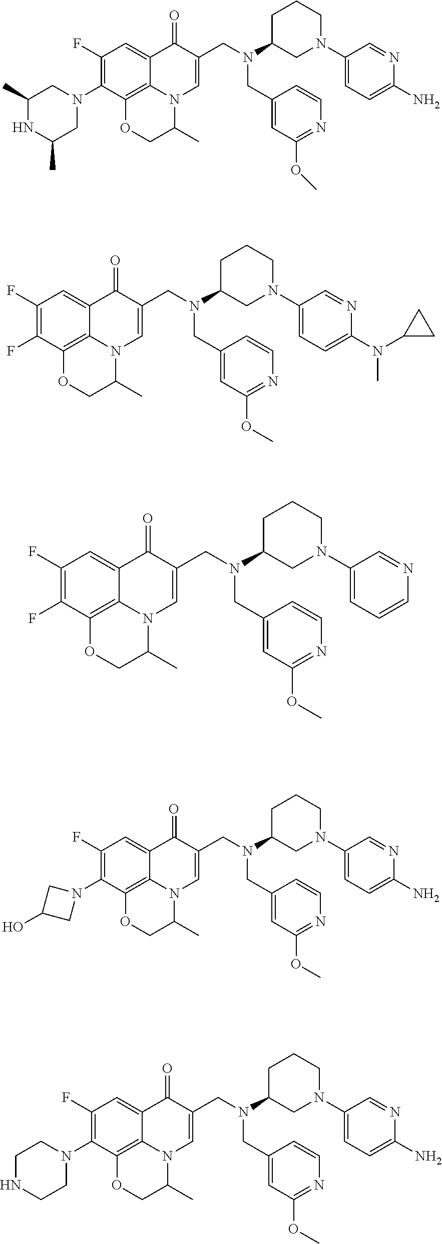 [SMILES: COc1cc(CN(Cc2cn3c4c(c(N5C[C@@H](C)N[C@@H](C)C5)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3C)[C@H]2CCCN(c3ccc(N(C)C4CC4)nc3)C2)ccn1 ; COc1cc(CN(Cc2cn3c4c(c(F)c(F)cc4c2=O)OCC3C)[C@H]2CCCN(c3cccnc3)C2)ccn1 ; COc1cc(CN(Cc2cn3c4c(c(N5CC(O)C5)c(F)cc4c2=O)OCC3C)[C@H]2CCCN(c3ccc(N)nc3)C2)ccn1 ; COc1cc(CN(Cc2cn3c4c(c(N5CCNCC5)c(F)cc4c2=O)OCC3C)[C@H]2CCCN(c3ccc(N)nc3)C2)ccn1]

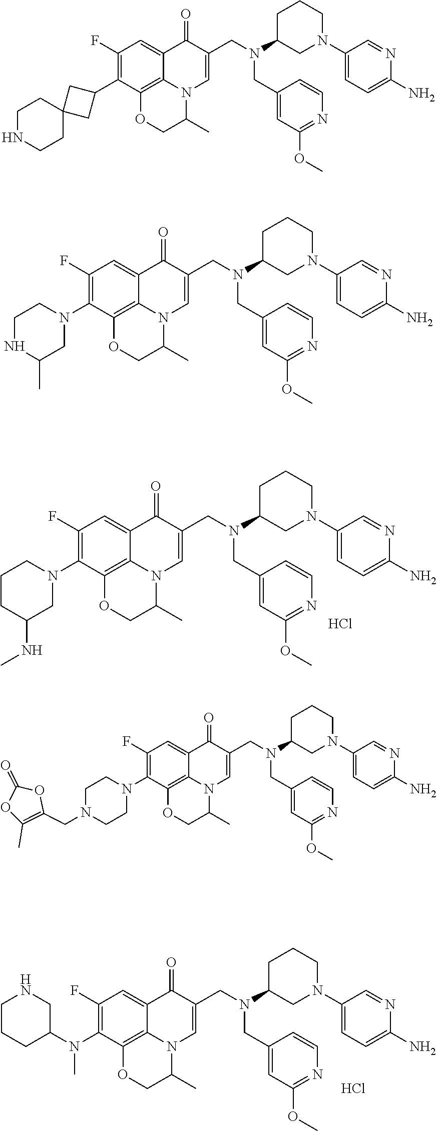

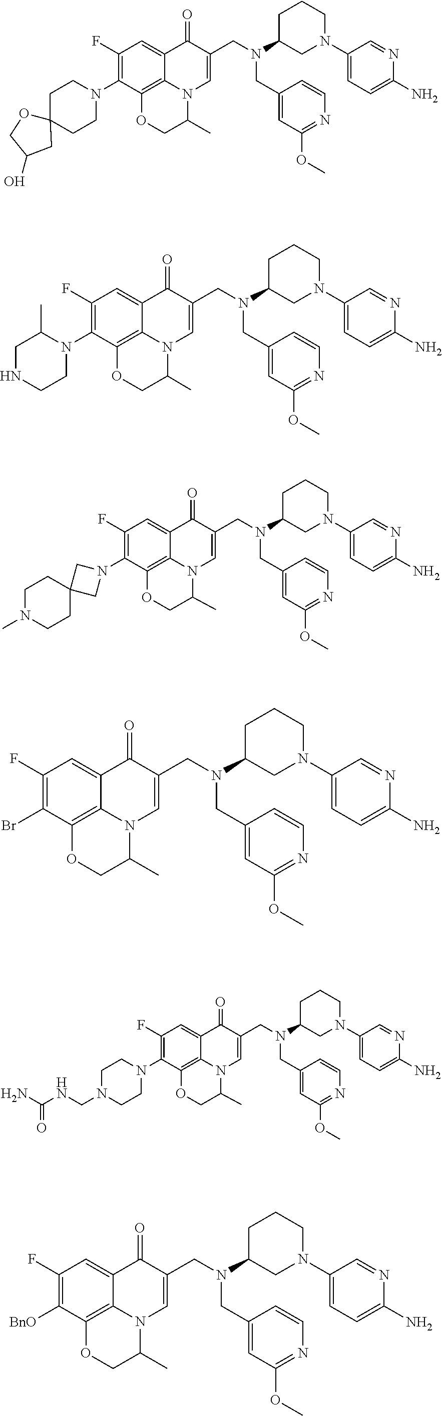

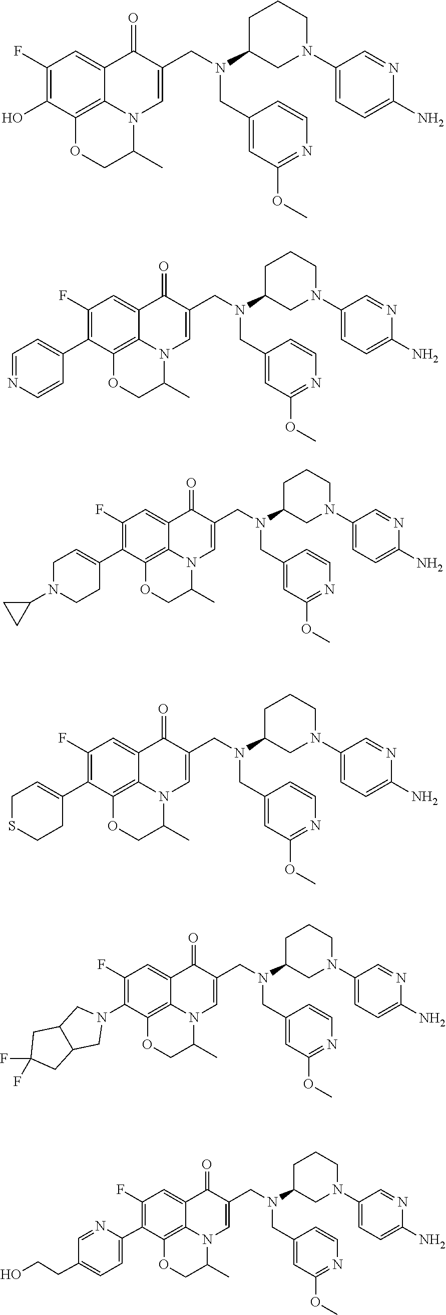

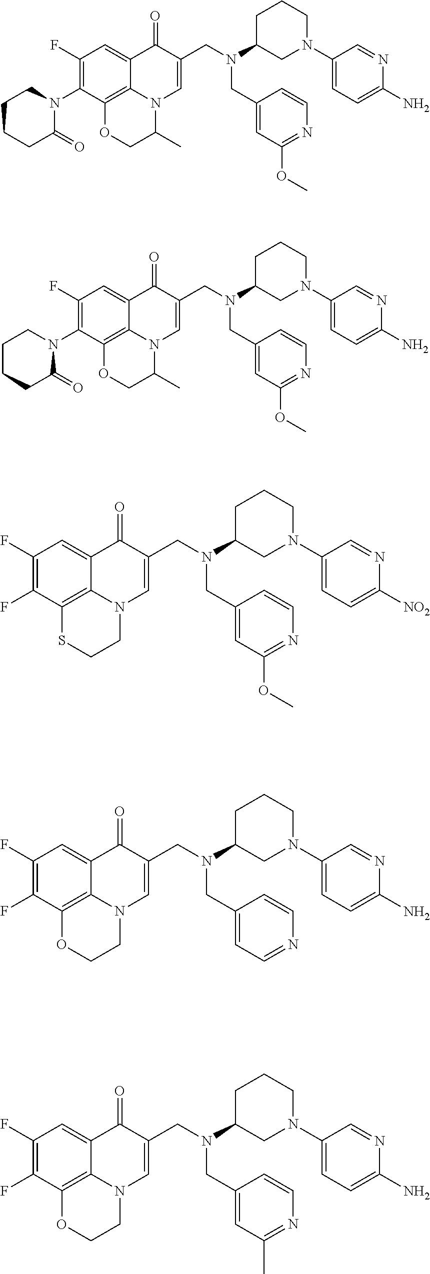

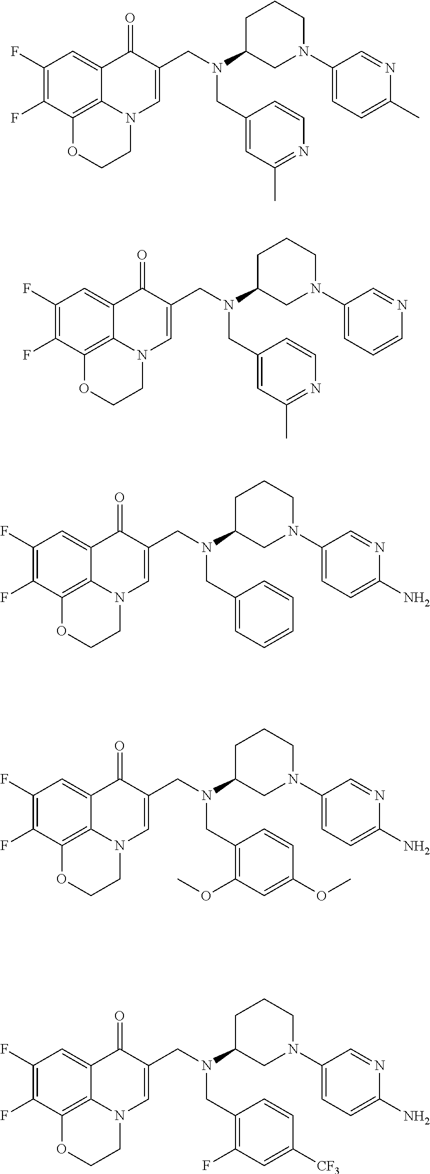

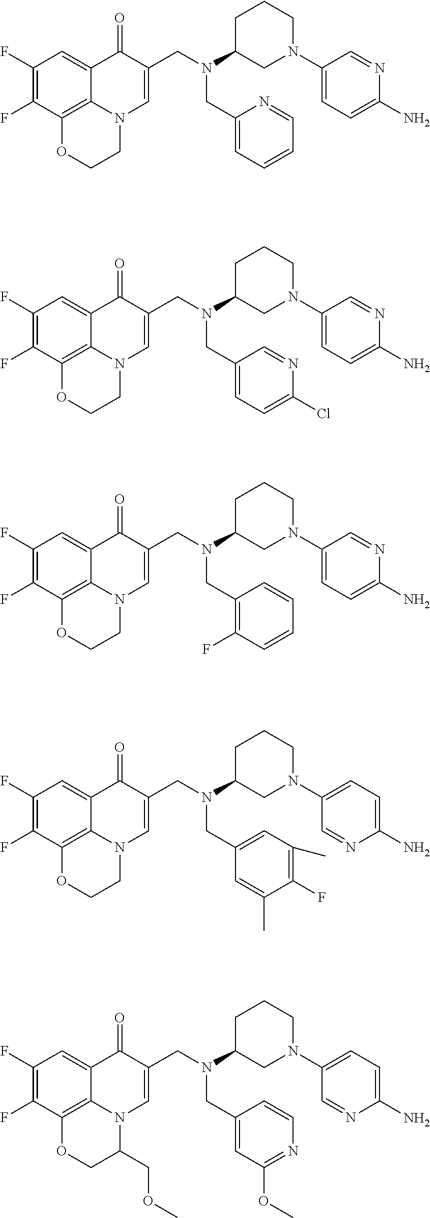 [SMILES: Nc1ccc(N2CCC[C@H](N(Cc3ccccn3)Cc3cn4c5c(c(F)c(F)cc5c3=O)OCC4)C2)cn1 ; Nc1ccc(N2CCC[C@H](N(Cc3ccc(Cl)nc3)Cc3cn4c5c(c(F)c(F)cc5c3=O)OCC4)C2)cn1 ; Nc1ccc(N2CCC[C@H](N(Cc3ccccc3F)Cc3cn4c5c(c(F)c(F)cc5c3=O)OCC4)C2)cn1 ; Cc1cc(CN(Cc2cn3c4c(c(F)c(F)cc4c2=O)OCC3)[C@H]2CCCN(c3ccc(N)nc3)C2)cc(C)c1F ; COCC1COc2c(F)c(F)cc3c(=O)c(CN(Cc4ccnc(OC)c4)[C@H]4CCCN(c5ccc(N)nc5)C4)cn1c23]

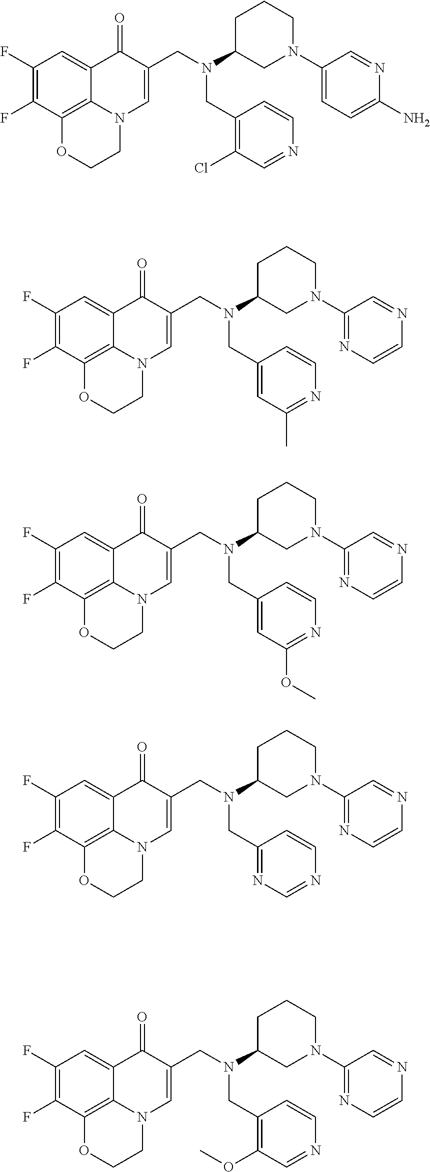

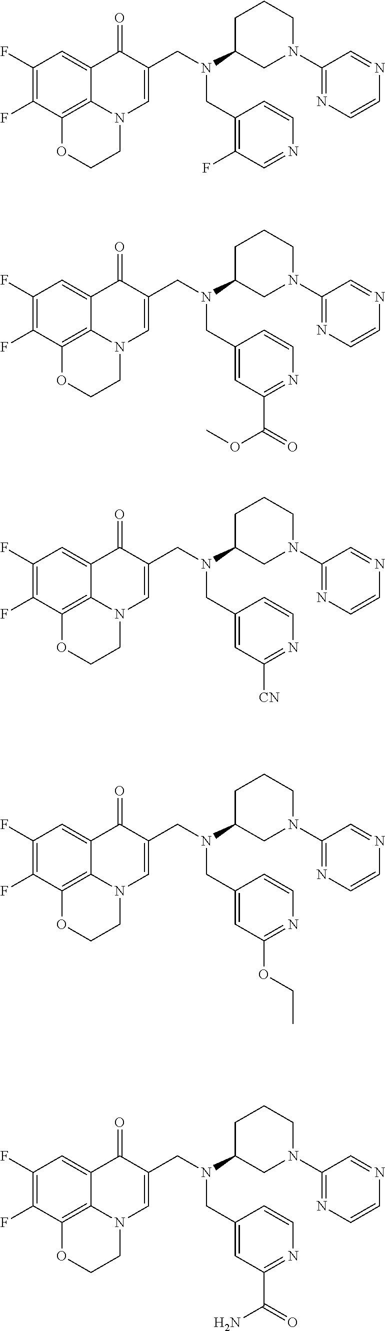 [SMILES: O=c1c(CN(Cc2ccncc2F)[C@H]2CCCN(c3cnccn3)C2)cn2c3c(c(F)c(F)cc13)OCC2 ; COC(=O)c1cc(CN(Cc2cn3c4c(c(F)c(F)cc4c2=O)OCC3)[C@H]2CCCN(c3cnccn3)C2)ccn1 ; N#Cc1cc(CN(Cc2cn3c4c(c(F)c(F)cc4c2=O)OCC3)[C@H]2CCCN(c3cnccn3)C2)ccn1 ; CCOc1cc(CN(Cc2cn3c4c(c(F)c(F)cc4c2=O)OCC3)[C@H]2CCCN(c3cnccn3)C2)ccn1 ; NC(=O)c1cc(CN(Cc2cn3c4c(c(F)c(F)cc4c2=O)OCC3)[C@H]2CCCN(c3cnccn3)C2)ccn1]

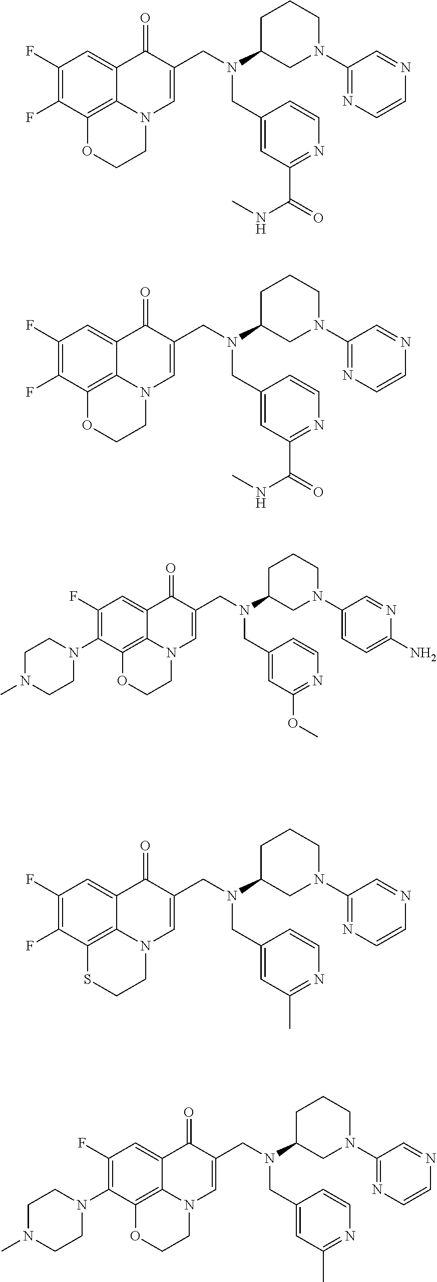 [SMILES: CNC(=O)c1cc(CN(Cc2cn3c4c(c(F)c(F)cc4c2=O)OCC3)[C@H]2CCCN(c3cnccn3)C2)ccn1 ; CNC(=O)c1cc(CN(Cc2cn3c4c(c(F)c(F)cc4c2=O)OCC3)[C@H]2CCCN(c3cnccn3)C2)ccn1 ; COc1cc(CN(Cc2cn3c4c(c(N5CCN(C)CC5)c(F)cc4c2=O)OCC3)[C@H]2CCCN(c3ccc(N)nc3)C2)ccn1 ; Cc1cc(CN(Cc2cn3c4c(c(F)c(F)cc4c2=O)SCC3)[C@H]2CCCN(c3cnccn3)C2)ccn1 ; Cc1cc(CN(Cc2cn3c4c(c(N5CCN(C)CC5)c(F)cc4c2=O)OCC3)[C@H]2CCCN(c3cnccn3)C2)ccn1]

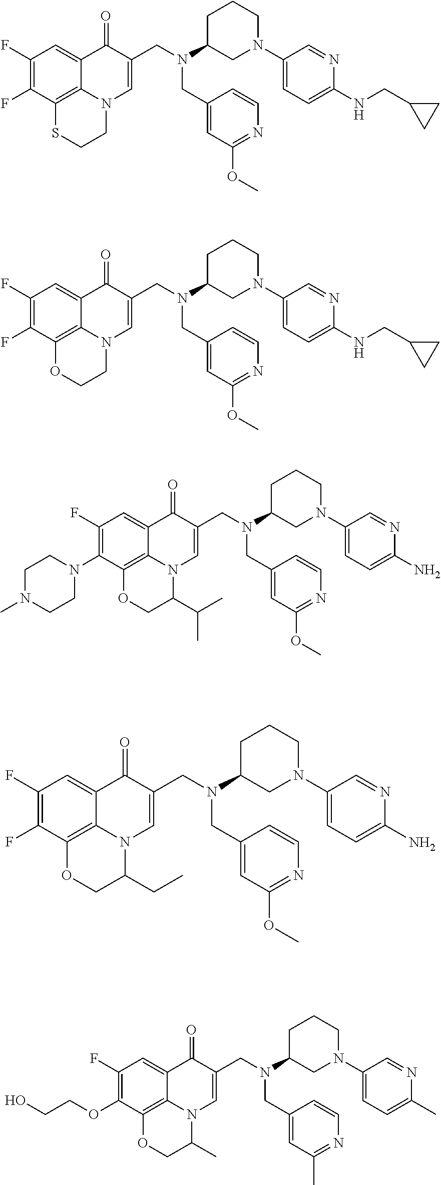 [SMILES: COc1cc(CN(Cc2cn3c4c(c(F)c(F)cc4c2=O)SCC3)[C@H]2CCCN(c3ccc(NCC4CC4)nc3)C2)ccn1 ; COc1cc(CN(Cc2cn3c4c(c(F)c(F)cc4c2=O)OCC3)[C@H]2CCCN(c3ccc(NCC4CC4)nc3)C2)ccn1 ; COc1cc(CN(Cc2cn3c4c(c(N5CCN(C)CC5)c(F)cc4c2=O)OCC3C(C)C)[C@H]2CCCN(c3ccc(N)nc3)C2)ccn1 ; CCC1COc2c(F)c(F)cc3c(=O)c(CN(Cc4ccnc(OC)c4)[C@H]4CCCN(c5ccc(N)nc5)C4)cn1c23 ; Cc1ccc(N2CCC[C@H](N(Cc3ccnc(C)c3)Cc3cn4c5c(c(OCCO)c(F)cc5c3=O)OCC4C)C2)cn1]

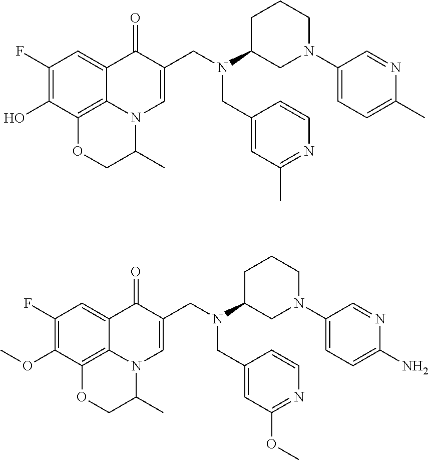

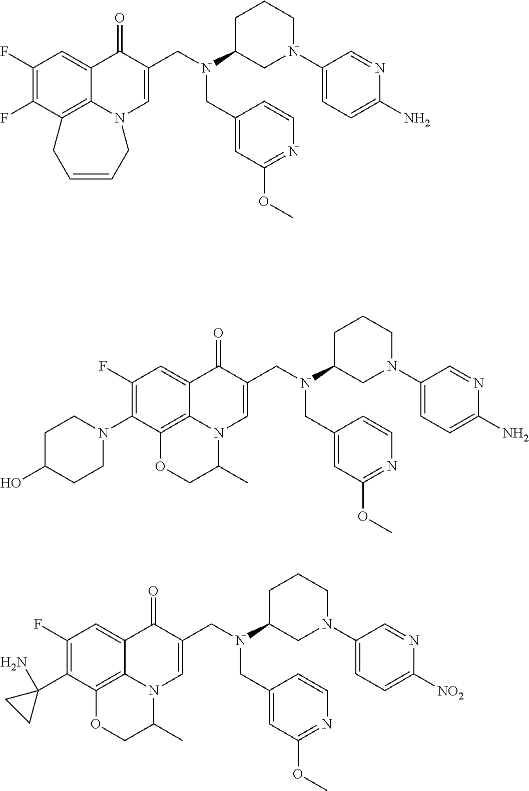 [SMILES: COc1cc(CN(Cc2cn3c4c(c(F)c(F)cc4c2=O)CC=CC3)[C@H]2CCCN(c3ccc(N)nc3)C2)ccn1 ; COc1cc(CN(Cc2cn3c4c(c(N5CCC(O)CC5)c(F)cc4c2=O)OCC3C)[C@H]2CCCN(c3ccc(N)nc3)C2)ccn1 ; COc1cc(CN(Cc2cn3c4c(c(C5(N)CC5)c(F)cc4c2=O)OCC3C)[C@H]2CCCN(c3ccc([N+](=O)[O-])nc3)C2)ccn1]

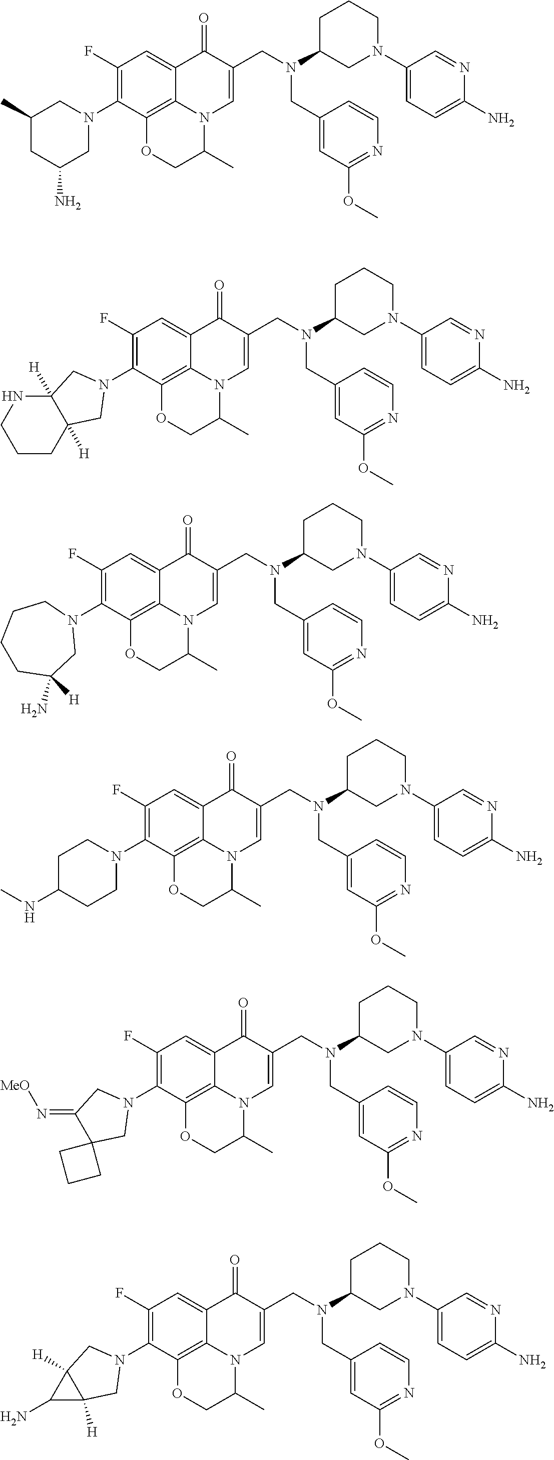

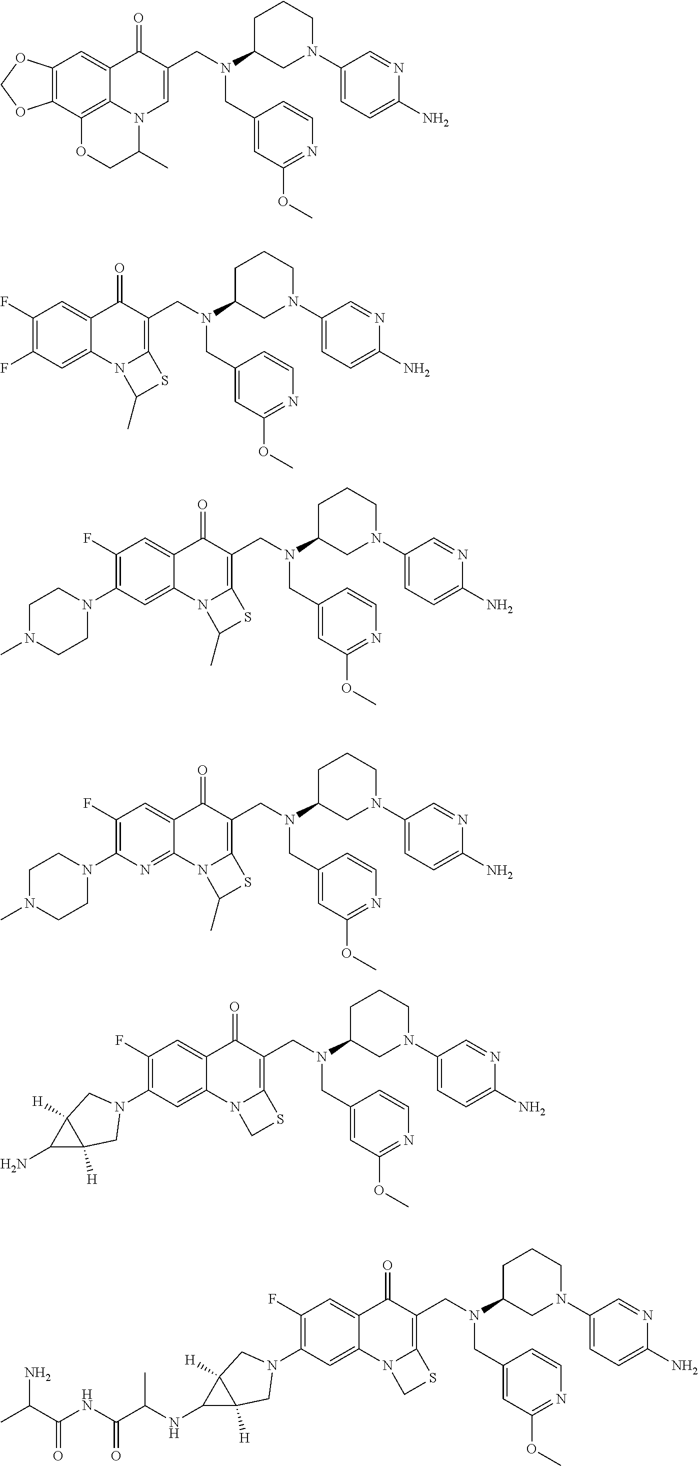

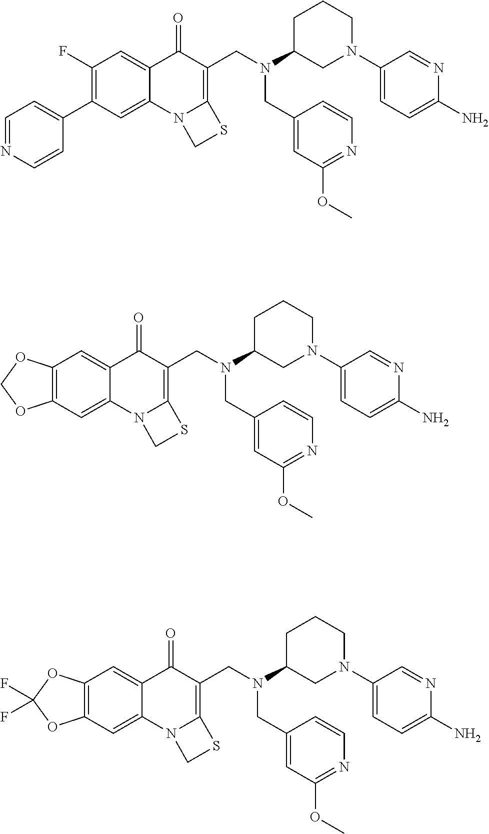 [SMILES: COc1cc(CN(Cc2c3n(c4cc(-c5ccncc5)c(F)cc4c2=O)CS3)[C@H]2CCCN(c3ccc(N)nc3)C2)ccn1 ; COc1cc(CN(Cc2c3n(c4cc5c(cc4c2=O)OCO5)CS3)[C@H]2CCCN(c3ccc(N)nc3)C2)ccn1 ; COc1cc(CN(Cc2c3n(c4cc5c(cc4c2=O)OC(F)(F)O5)CS3)[C@H]2CCCN(c3ccc(N)nc3)C2)ccn1]

or a pharmaceutically acceptable form or an isotope derivative thereof.

In certain embodiments, a compound of the invention has one or more (e.g., 1, 2, 3) deuterium atoms replacing one or more (e.g., 1, 2, 3) hydrogen atoms. In certain embodiments, a compound of the invention has one deuterium atom replacing one hydrogen atom.

In another aspect, the invention generally relates to a pharmaceutical composition comprising a compound disclosed herein, effective to treat or reduce one or more diseases or disorders, in a mammal, including a human.

In yet another aspect, the invention generally relates to a pharmaceutical composition comprising a compound of structural formula I or formula II.

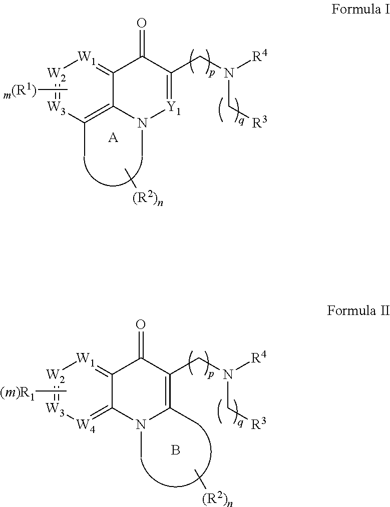

wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein R¹ is optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
   or a pharmaceutically acceptable form or an isotope derivative thereof, effective to treat or reduce one or more diseases or disorders, in a mammal, including a human, and a pharmaceutically acceptable excipient, carrier, or diluent.

In yet another aspect, the invention generally relates to a pharmaceutical composition comprising a compound of structural formula III:

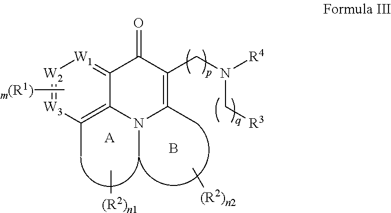

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
   or a pharmaceutically acceptable form or an isotope derivative thereof, effective to treat or reduce one or more diseases or disorders, in a mammal, including a human.

In certain embodiments, the pharmaceutical composition is suitable for systematic administration.

In certain embodiments, the pharmaceutical composition is useful to treat or reduce cancer.

In certain embodiments, the pharmaceutical composition is useful to treat or reduce breast, colon, endometrial, kidney, lung, melanoma, prostate, thyroid cancer or leukemia.

In certain embodiments, the pharmaceutical composition is useful to treat or reduce an infectious disease or disorder.

In certain embodiments, the pharmaceutical composition is useful to treat or reduce an autoimmune disease or disorder.

In certain embodiments, the pharmaceutical composition is useful to treat or reduce the effect of aging.

In yet another aspect, the invention generally relates to a unit dosage form comprising a pharmaceutical composition disclosed herein.

In certain embodiments, the unit dosage form is suitable for systematic administration.

In certain embodiments, the unit dosage form is an injectable, a solution, a suspension, a tablet or a capsule.

In certain embodiments, the unit dosage form is suitable for intravenous administration.

In certain embodiments, the unit dosage form is in the form of a liquid formulation.

In yet another aspect, the invention generally relates to a method for treating or reducing a disease or disorder, comprising administering to a subject in need thereof a pharmaceutical composition comprising a compound having the structural formula I or formula II:

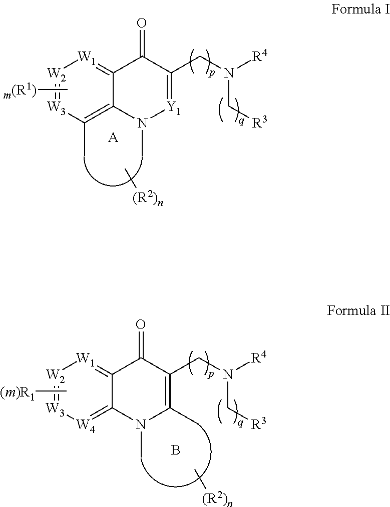

wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein R¹ is optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
   or a pharmaceutically acceptable form or an isotope derivative thereof, effective to treat cancer, or a related disease or disorder, in a mammal, including a human.

In yet another aspect, the invention generally relates to a method for treating or reducing a disease or disorder, comprising administering to a subject in need thereof a pharmaceutical composition comprising a compound having the structural formula III:

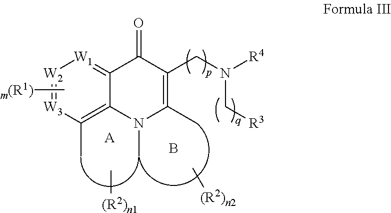

wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
   or a pharmaceutically acceptable form or an isotope derivative thereof, effective to treat cancer, or a related disease or disorder, in a mammal, including a human.

In certain embodiments, the disease or disorder is mediated by or associated with STING.

In certain embodiments, the disease or disorder is selected from cancer and pre-cancerous syndromes.

In certain embodiments, the disease or disorder is an infectious disease or disorder.

In certain embodiments, the disease or disorder is an autoimmune disease or disorder.

In certain embodiments, the method of the invention treats or reduces the effect of aging.

In certain embodiments, the cancer that may be treated is selected from breast, colon, endometrial, kidney, lung, melanoma, prostate, thyroid cancer and leukemias. Other PTEN-deficient neoplasm may also be treated with the compounds and pharmaceutical compositions of the invention, for example, brain (gliomsa), glioblastomas, Bannayan-Zonana syndrome, Cowden disease, Lhermitte-Duclos disease, colorectal cancer, Wilm's tumor, Ewing's sarcoma, Rhabdomyosarcoma, ependymoma, medulloblastoma, head and neck cancer, liver cancer, squamous cell carcinoma, ovarian cancer, pancreatic cancer, sarcoma cancer, osteosarcoma, giant cell tumor of bone, lymphoblastic T cell, malignant lymphoma, hodykins lymphoma, non-hodgkins lymphoma, lymphoblastic T cell lymphoma, Burkitt's lymphoma, follicylar lymphoma, neuroblastoma, bladder cancer, urothelial cancer, vulval cancer, cervical cancer, renal cancer, mesothelioma, esophageal cancer, salivary gland cancer, hepatocellular cancer, gastric cancer, nasopharangeal cancer, buccal cancer, cancer of the mouth, gastrointestinal stromal tumor and testicular cancer.

In yet another aspect, the invention generally relates to use of a compound disclosed herein, and a pharmaceutically acceptable excipient, carrier, or diluent, in preparation of a medicament for treating a disease or disorder.

In certain embodiments, the disease or disorder is cancer, or a related disease or disorder.

In certain embodiments, the cancer that may be treated is selected from breast, colon, endometrial, kidney, lung, melanoma, prostate, thyroid cancer and leukemia.

In certain embodiments, the medicament is for oral administration.

In certain embodiments, the medicament is for intravenous administration.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (3H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Examples

The following examples are given for the purpose of illustrating the invention, but not for limiting the scope or spirit of the invention.

Compounds of the invention, including those specifically disclosed herein above and herein below, may be prepared as described in the following schemes. For example, the compounds of Formula (I), Formula (II) and Formula (III) may be prepared as described in Schemes below, which are known to those of skill in the art for making fragments and combinations thereof. Although the present invention has been described in detail with preferred embodiments, those of ordinary skill in the art should understand that modifications, variations, and equivalent replacements made to the present invention within the scope of the present invention belong to the protection of the present invention.

### Abbreviations

| Abbreviation | Name |
|---|---|
| DCE | 1,2-Dichlorethane |
| DCM | Dichloromethane |
| DMSO | Dimethyl sulfoxide |
| HPLC | High-performance liquid chromatography |
| LCMS | Liquid chromatography-mass spectrometry |
| MS | Mass spectrometry |
| TLC | Thin-layer chromatography |
| EtOAc | Ethyl acetate |
| Pd₂(dba)₃ | Tri (dibenzylidene acetone) dipalladium |
| Xantphos | (5-diphenylphosphanyl-9,9-dimethyl-xanthen-4-yl)- |
| | diphenyl-phosphane |
| i-PrOH | Isopropanol |
| Pd/C | Palladium on carbon |
| DIPEA | N-ethyl-N-isopropyl-propan-2-amine |
| (DIEA) | |
| DMF | N,N-dimethyl formamide |
| Ac₂O | Acetyl acetate |
| NMP | 1-methylpyrrolidin-2-one |
| UHP | Urea Hydrogen Peroxide |
| HATU | O-(7-azobentriazole-1-yl)-N,N,N, N-tetramethylurea |
| | hexafluorophosphine |

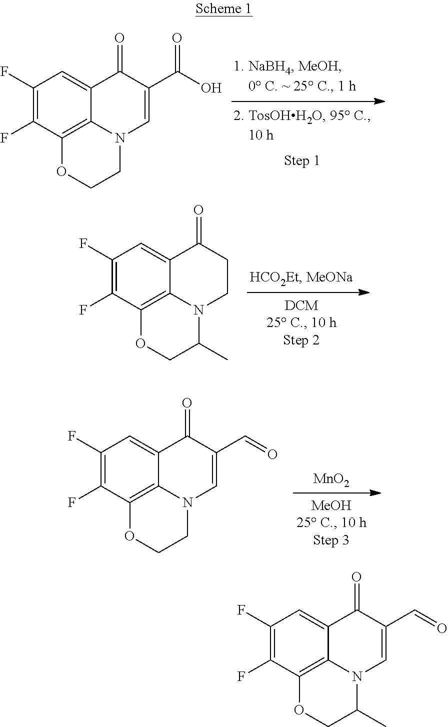

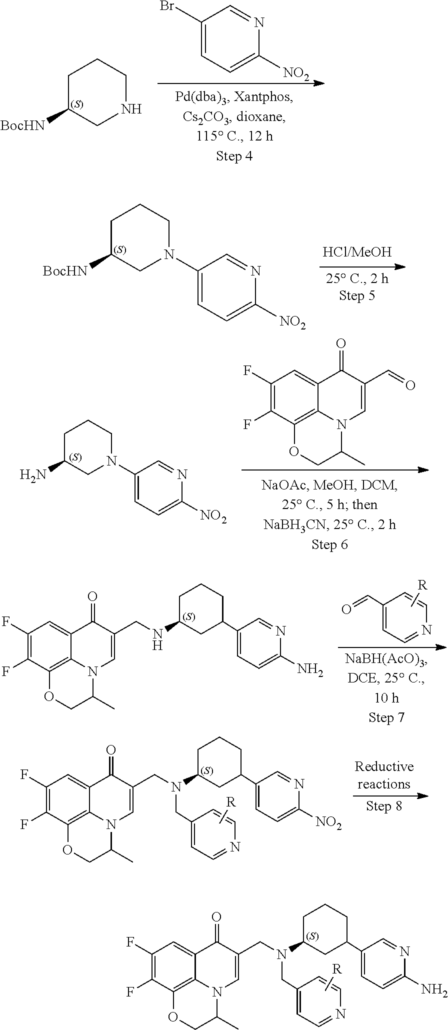

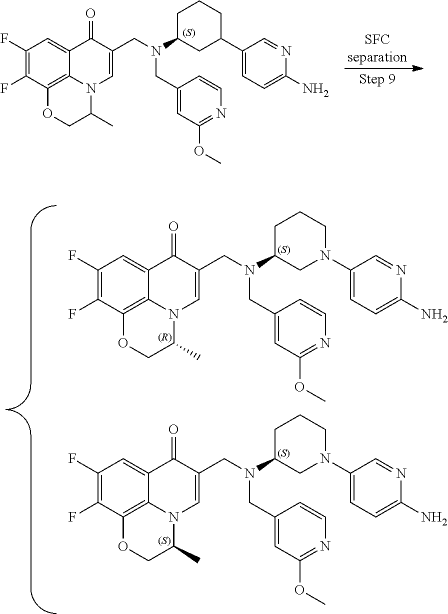

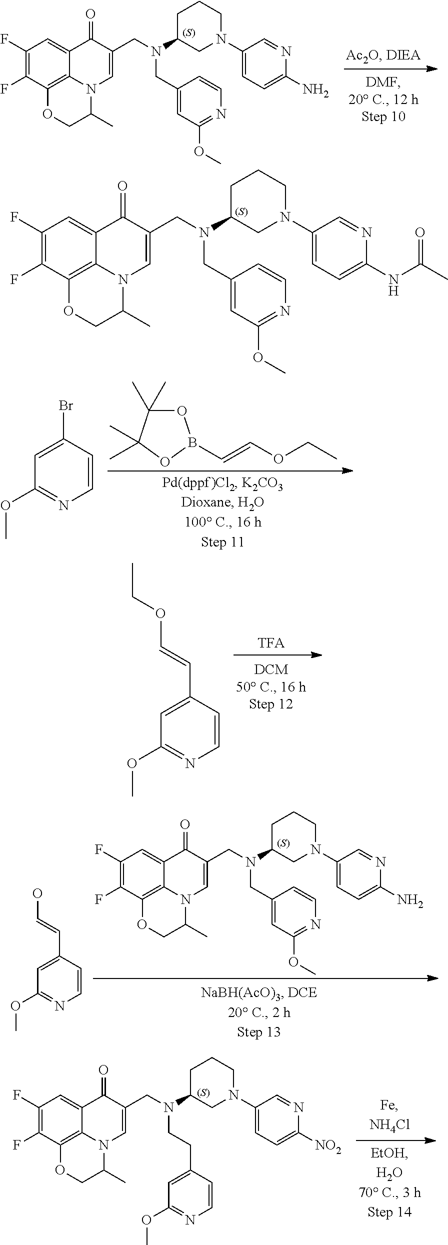

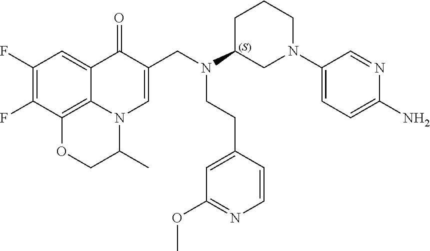

### General Procedures for Preparing Compounds in Scheme 1

### Preparation of 9,10-difluoro-3-methyl-5,6-dihydro-2H-[1,4]oxazino[2,3,4-ij]quinolin-7(3H)-one (Step 1 in Scheme 1)

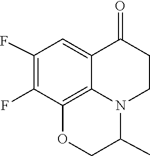 [SMILES: CC1COc2c(F)c(F)cc3c2N1CCC3=O]

To a solution of 6,7-difluoro-2-methyl-0-oxo-4-oxa-1-azatricyclo[7.3.1.0^{5,13}]trideca-5(13),6,8, 11-tetraene-11-carboxylic acid (10 g, 35.56 mmol, 1 eq) in MeOH (150 mL) was added NaBH₄ (6.05 g, 160.02 mmol, 4.5 eq) in portions at 0° C. during half an hour. Then the mixture was stirred at 25° C. for half an hour under N₂ atmosphere. Then TsOH·H₂O (676.42 mg, 3.56 mmol, 0.1 eq) was added and the mixture was heated at 95° C. for 10 hours under N₂ atmosphere. LCMS showed the reaction was nearly complete. The reaction mixture was concentrated under reduced pressure. The residue was quenched with water (100 mL) slowly at 0° C. and extracted with EtOAc (60 mL×3). The combined organic layer was washed with brine (30 mL×1), dried over Na₂SO₄, filtered and concentrated under reduced pressure. The residue was purified by flash silica gel chromatography (Biotage®; 80 g SepaFlash® Silica Flash Column, Eluent of 0-42% gradient ethyl acetate/petroleum ether @100 mL/min.). The eluent was removed under reduced pressure. Compound 6,7-difluoro-2-methyl-4-oxa-1-azatricyclo [7.3.1.0^{5,13}]trideca-5(13),6,8-trien-10-one (6.4 g, 26.75 mmol, 75.23% yield) was obtained as yellow solid. ¹H NMR (DMSO-d₆, 400 MHz) δ 7.17-7.12 (m, 1H), 4.29-4.23 (m, 2H), 3.59˜3.54 (m, 2H), 3.33˜3.31 (m, 1H), 2.71˜2.63 (m, 2H), 1.16 (d, J=6.4 Hz, 3H).

### Preparation of 9,10-difluoro-3-methyl-7-oxo-3,5,6,7-tetrahydro-2H-[1,4]oxazino[2,3,4-ij]quinoline-6-carbaldehyde (Step 2 in Scheme 1)

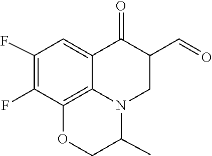 [SMILES: CC1COc2c(F)c(F)cc3c2N1CC(C=O)C3=O]

To a mixture of 6,7-difluoro-2-methyl-4-oxa-1-azatricyclo[7.3.1.0^{5,13}]trideca-5(13),6,8-trien-10-one (6.4 g, 26.75 mmol, 1 eq) in DCM (150 mL) was added NaOMe (11.27 g, 104.34 mmol, 50% purity, 3.9 eq) in portions at 0° C. The mixture was stirred at 25° C. for half an hour, then ethyl formate (7.73 g, 104.34 mmol, 8.39 mL, 3.9 eq) was added dropwise at 25° C. Then the mixture was stirred at 25° C. for 10 hours under N₂ atmosphere. LCMS showed the reaction was complete. The reaction mixture was poured into ice water (100 mL) slowly at 0° C. and made pH=6 with 0.5N HCl. The organic layer was separated and the aqueous was extracted with DCM (100 mL×2). The combined organic layer was washed with brine (100 mL×1), dried over Na₂SO₄, filtered and concentrated under reduced pressure. Compound 6,7-difluoro-2-methyl-10-oxo-4-oxa-1-azatricyclo[7.3.1.0^{5,13}]trideca-5(13),6,8-triene-11-carbaldehyde (7 g, crude) was obtained as pale brown oil. ¹H NMR (DMSO-d₆, 400 MHz) δ 11.54 (s, 1H), 7.77 (s, 1H), 7.16˜7.11 (m, 1H), 4.23˜4.13 (m, 3H), 3.89˜3.84 (m, 1H), 3.49˜3.17 (m, 1H), 1.19 (d, J=6.4 Hz, 3H).

### Preparation of 9,10-difluoro-3-methyl-7-oxo-3,7-dihydro-2H-[1,4]oxazino[2,3,4-ij]quinoline-6-carbaldehyde (Step 3 in Scheme 1)

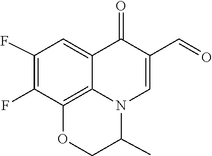 [SMILES: CC1COc2c(F)c(F)cc3c(=O)c(C=O)cn1c23]

To a mixture of 6,7-difluoro-2-methyl-10-oxo-4-oxa-1-azatricyclo[7.3.1.0^{5,13}]trideca-5(13), 6,8-triene-11-carbaldehyde (7 g, 26.19 mmol, 1 eq) in MeOH (150 mL) was added MnO₂ (11.37 g, 130.97 mmol, 5 eq). Then the mixture was stirred at 25° C. for 10 hours under N₂ atmosphere. LCMS showed the reaction was complete. The reaction mixture was filtered through a pad of the Celite and the filtrate was discarded. The filter cake was washed with DCM (200 mL×2). The filtrate was concentrated under reduced pressure. The residue was triturated with DCM and MeOH (v:v=1:3, 30 mL). After filtration, the filter cake was collected. Compound 6,7-difluoro-2-methyl-10-oxo-4-oxa-1-azatricyclo[7.3.1.0^{5,13}]trideca-5(13),6,8,11-tetraene-11-carbaldehyde (6.5 g, 24.51 mmol, 93.56% yield) was obtained as off white solid. ¹H NMR (DMSO-d₆, 400 MHz) δ 10.16 (s, 1H), 8.65 (s, 1H), 7.73˜7.68 (m, 1H), 4.86-4.65 (m, 1H), 4.62˜4.61 (m, 1H), 4.47˜4.45 (m, 1H), 1.44 (d, J=6.8 Hz, 3H).

### Preparation of (S)-tert-butyl (1-(6-nitropyridin-3-yl)piperidin-3-yl)carbamate (Step 4 in Scheme 1)

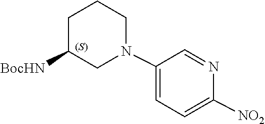

A mixture of tert-butyl N-(3-piperidyl)carbamate (10.26 g, 51.23 mmol, 1.3 eq), 5-bromo-2-nitro-pyridine (8 g, 39.41 mmol, 1 eq), Pd₂(dba)₃ (1.80 g, 1.97 mmol, 0.05 eq), Xantphos (1.37 g, 2.36 mmol, 0.06 eq) and Cs₂CO₃ (17.33 g, 53.20 mmol, 1.35 eq) in dioxane (120 mL) was degassed and purged with N₂ for 3 times, and then the mixture was stirred at 115° C. for 12 hours under N₂ atmosphere. LCMS showed the reaction was complete. The mixture was concentrated under reduced pressure. To the residue was added EtOAc (50 mL). After filtration through a pad of the Celite, the filter cake was washed with EtOAc (70 mL). The combined filtrate was concentrated under reduced pressure. The residue was purified by flash silica gel chromatography (Biotage; 120 SepaFlash® Silica Flash Column, Eluent of 0˜47% ethyl acetate/petroleum ether gradient @45 mL/min). The eluent was removed under reduced pressure. Compound tert-butyl N-[1-(6-nitro-3-pyridyl)-3-piperidyl]carbamate (7.23 g, 22.43 mmol, 56.91% yield) was obtained as yellow solid. ¹H NMR (DMSO-d₆, 400 MHz) δ 8.21 (s, 1H), 8.13 (d, J=9.2 Hz, 1H), 7.41 (d, J=6.4 Hz, 1H), 7.01 (d, J=6.8 Hz, 1H), 3.89-3.87 (m, 2H), 3.46˜3.37 (m, 1H), 3.14˜3.07 (m, 1H), 3.04˜3.01 (m, 1H), 1.87˜1.82 (m, 1H), 1.78˜1.73 (m, 1H), 1.54˜1.50 (m, 2H), 1.40 (s, 9H).

### Preparation of (S)-1-(6-nitropyridin-3-yl)piperidin-3-amine (Step 5 in Scheme 1)

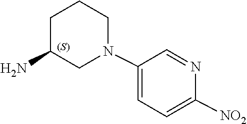

To a solution of tert-butyl N-[1-(6-nitro-3-pyridyl)-3-piperidyl]carbamate (7.1 g, 22.03 mmol, 1 eq) in MeOH (7 mL) was added HCl/MeOH (4 M, 40 mL, 7.26 eq). The reaction mixture was stirred at 25° C. for 2 hours. LCMS showed the reaction was complete. The mixture was concentrated under reduced pressure. Compound 1-(6-nitro-3-pyridyl)piperidin-3-amine (6.8 g, 20.51 mmol, 93.10% yield, 3HCl) was obtained as white solid. ¹H NMR (DMSO-d₆, 400 MHz) δ 8.52 (s, 2H), 8.28 (s, 1H), 8.16 (d, J=8.8 Hz, 1H), 7.50 (d, J=9.2 Hz, 1H), 4.12-4.09 (m, 1H), 3.83˜3.79 (m, 1H), 3.44˜3.39 (m, 1H), 3.24˜3.21 (m, 2H), 2.04˜2.01 (m, 1H), 1.82˜1.81 (m, 1H), 1.74˜1.70 (m, 1H), 1.58˜1.55 (s, 1H).

### Preparation of 9,10-difluoro-3-methyl-6-((((S)-1-(6-nitropyridin-3-yl)piperidin-3-yl)amino)methyl)-2H-[1,4]oxazino[2,3,4-ij]quinolin-7(3H)-one and 9-fluoro-10-methoxy-3-methyl-6-((((S)-1-(6-nitropyridin-3-yl)piperidin-3-yl)amino)methyl)-2,3-dihydro-7H-[1,4]oxazino[2,3,4-ij]quinolin-7-one (Step 6 in Scheme 1)

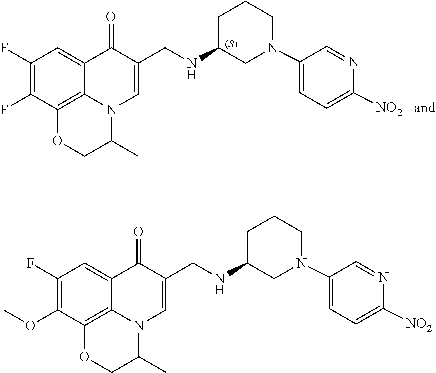

A solution of 6,7-difluoro-2-methyl-10-oxo-4-oxa-1-azatricyclo[7.3.1.0^{5,13}]trideca-5(13),6,8,11-tetraene-11-carbaldehyde (1 g, 3.77 mmol, 1 eq), AcONa (1.55 g, 18.85 mmol, 5 eq) and (3S)-1-(6-nitro-3-pyridyl)piperidin-3-amine (1.25 g, 3.77 mmol, 1 eq, 3HCl) in MeOH (10 mL) and DCM (10 mL) was stirred at 25° C. for 5 hours. Then NaBH₃CN (355.41 mg, 5.66 mmol, 1.5 eq) was added in portions at 0° C. and the mixture was stirred at 25° C. for 2 hours. LCMS and TLC showed the reaction was complete. The reaction mixture was quenched with ice water (20 mL) at 0° C. and made pH=8 with sat. NaHCO₃. There was some yellow solid formed. The solid was discarded after filtration. The filtrate was extracted with DCM and i-PrOH (v:v=3:1, 50 mL×2). The combined organic layer was washed with brine (20 mL×1), dried over Na₂SO₄, filtered and concentrated under reduced pressure. Compound 6,7-difluoro-2-methyl-11-[[[(3S)-1-(6-nitro-3-pyridyl)-3-piperidyl]amino]methyl]-4-oxa-1-azatricyclo[7.3.1.0^{5,13}]trideca-5(13),6,8,11-tetraen-10-one (1.9 g, crude) was obtained as yellow solid. ¹H NMR (DMSO-d₆, 400 MHz) δ 8.23 (s, 1H), 8.22˜8.06 (m, 2H), 7.56 (t, J=7.6 Hz, 1H), 7.46-7.43 (m, 1H), 4.60˜4.54 (m, 2H), 4.47˜4.40 (m, 1H), 4.05˜4.00 (m, 1H), 3.87-3.81 (m, 1H), 3.67-3.60 (m, 2H), 3.17˜3.14 (m, 1H), 3.07˜2.89 (m, 1H), 2.60˜2.53 (m, 1H), 1.97˜1.91 (m, 1H), 1.77-1.70 (m, 1H), 1.44-1.40 (m, 2H), 1.37-1.35 (m, 3H). Compound 9-fluoro-10-methoxy-3-methyl-6-((((S)-1-(6-nitropyridin-3-yl)piperidin-3-yl)amino)methyl)-2,3-dihydro-7H-[1,4]oxazino[2,3,4-ij]quinolin-7-one (190 mg, crude) was obtained as yellow solid.

### Preparation of Compounds in Scheme 1 (Step 7 in Scheme 1)

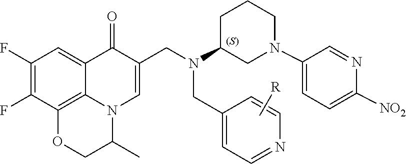

A solution of 6,7-difluoro-2-methyl-11-[[[(3S)-1-(6-nitro-3-pyridyl)-3-piperidyl]amino]methyl]-4-oxa-1-azatricyclo[7.3.1.0^{5,13}]trideca-5(13),6,8,11-tetraen-10-one (901.46 umol, 1 eq) and different aldehyde (0.901 mmol˜1.35 mmol, 1.0 eq˜1.5 eq) in DCE (13 mL/mmol 22 mL/mmol) was stirred at 25° C. for half an hour˜2 hours. Then the mixture was cooled to 0° C., NaBH(OAc)₃ (1.35 mmol˜1.80 mmol, 1.5 eq˜2 eq) was added in portions at 0° C. The reaction mixture was stirred at 25° C. for 5 hours˜15 hours. LCMS and TLC showed the reaction was complete. The reaction mixture was quenched with ice water at 0° C. and made pH=8 with sat. NaHCO₃. The mixture was extracted with DCM and i-PrOH (v:v=3:1). The combined organic layer was washed with brine, dried over Na₂SO₄, filtered and concentrated under reduced pressure to give the desired product.

### Preparation of Final Compounds in Scheme 1 (Step 8 in Scheme 1)

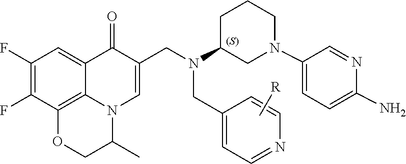

A mixture of 6,7-difluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.0^{5,13}]trideca-5(13),6,8,11-tetraen-10-one (675.00 umol, 1 eq) and Pd/C (10% purity, 20% wt) in MeOH (50 mL/mmol 130 mL/mmol) was stirred at 25° C. for 3 hours˜5 hours under H₂ (14.6 psi). Or to a mixture of 11-[[(5-chloro-2-methoxy-4-pyridyl)methyl-[(3S)-1-(6-nitro-3-pyridyl)-3-piperidyl]amino]methyl]-6,7-difluoro-2-methyl-4-oxa-1-azatricyclo[7.3.1.0^{5,13}]trideca-5(13),6,8,11-tetraen-10-one (159.48 umol, 1 eq) in MeOH (62 mL/mmol) and H₂O (21 mL/mmol) was added Fe (797.40 umol, 5 eq) and NH₄Cl (318.96 umol, 2 eq). The reaction mixture was stirred at 80° C. for half an hour. The mixture was filtered through a pad of the Celite. The filtrate was concentrated under reduced pressure. The residue was purified by prep-HPLC (column: Phenomenex C18 80*40 mm*3 um or Phenomenex C18 75*30 mm*3 um or Waters Xbridge BEH C18 100*30 mm*10 um; mobile phase: [water (NH₄HCO₃)-MeCN]; B %: 20%-65%, 8 mins). The eluent was removed under freeze drying. The aqueous solution was lyophilized to give the desired product.

### Compound 1

### Preparation of 9,10-difluoro-6-((((2-methoxypyridin-4-yl)methyl)((S)-1-(6-nitropyridin-3-yl)piperidin-3-yl)amino)methyl)-3-methyl-2H-[1,4]oxazino[2,3,4-ij]quinolin-7(3H)-one (Step 7 in Scheme 1)

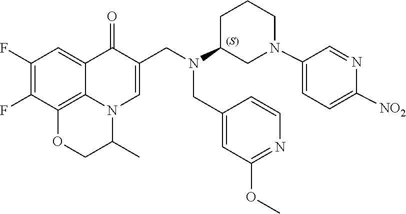

A solution of 6,7-difluoro-2-methyl-11-[[[(3S)-1-(6-nitro-3-pyridyl)-3-piperidyl]amino]methyl]-4-oxa-1-azatricyclo[7.3.1.0^{5,13}]trideca-5(13),6,8,11-tetraen-10-one (0.5 g, 901.46 umol, 85% purity, 1 eq) and 2-methoxypyridine-4-carbaldehyde (185.43 mg, 1.35 mmol, 1.5 eq) in DCE (20 mL) was stirred at 25° C. for half an hour. Then the mixture was cooled to 0° C., NaBH(OAc)₃ (382.11 mg, 1.80 mmol, 2 eq) was added in portions at 0° C. The mixture was stirred at 25° C. for 10 hours. LCMS and TLC showed the reaction was complete. The reaction mixture was quenched with ice water (20 mL) at 0° C. and made pH=8 with sat. NaHCO₃. The mixture was extracted with DCM and i-PrOH (v:v=3:1, 30 mL×3). The combined organic layer was washed with brine (20 mL×1), dried over Na₂SO₄, filtered and concentrated under reduced pressure. Compound 6,7-difluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.0^{5,13}]trideca-5(13),6,8, 11-tetraen-10-one (0.5 g, crude) was obtained as yellow solid. ¹H NMR (DMSO-d₆, 400 MHz) δ 8.25-8.24 (m, 1H), 8.12˜8.00 (m, 3H), 7.56-7.50 (m, 1H), 7.47-7.44 (m, 1H), 6.90-6.88 (m, 1H), 6.73 (s, 1H), 4.65-4.62 (m, 1H), 4.60-4.58 (m, 1H), 4.48˜4.23 (m, 2H), 4.05˜4.02 (m, 2H), 3.93˜3.90 (m, 2H), 3.83 (s, 3H), 3.77˜3.67 (m, 2H), 3.27˜3.19 (m, 1H), 3.04˜2.91 (m, 1H), 1.84˜1.61 (m, 2H), 1.39˜1.34 (m, 2H), 1.33˜1.31 (m, 3H).

### Preparation of 6-((((S)-1-(6-aminopyridin-3-yl)piperidin-3-yl)((2-methoxypyridin-4-yl)methyl)amino)methyl)-9,10-difluoro-3-methyl-2H-[1,4]oxazino[2,3,4-ij]quinolin-7(3H)-one (Step 8 in Scheme 1)

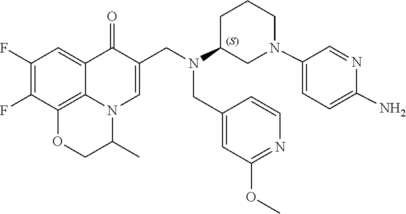

A mixture of 6,7-difluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.0^{5,13}]trideca-5(13),6,8,11-tetraen-10-one (0.4 g, 675.00 umol, 1 eq) and Pd/C (0.1 g, 10% purity) in MeOH (100 mL) was stirred at 25° C. for 5 hours under H₂ (14.6 psi). LCMS and HPLC showed the reaction was complete. The mixture was filtered through a pad of the Celite. The filtrate was concentrated under reduced pressure. The residue was purified by prep-HPLC (column: Phenomenex C18 80*40 mm*3 um; mobile phase: [water (NH₄HCO₃)-MeCN]; B %: 20%-50%, 8 min). The eluent was removed under freeze drying. Compound 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (186.3 mg, 326.76 umol, 48.41% yield, 98.68% purity) was obtained as off white solid. ¹H NMR (DMSO-d₆, 400 MHz) δ 8.08 (d, J=6.8 Hz, 1H), 7.99 (d, J=5.2 Hz, 1H), 7.61 (d, J=3.2 Hz, 1H), 7.56-7.51 (m, 1H), 7.16-7.13 (m, 1H), 7.01˜6.99 (m, 1H), 6.82 (s, 1H), 6.39-6.36 (m, 1H), 5.37 (s, 2H), 4.71˜4.69 (m, 1H), 4.57˜4.55 (m, 1H), 4.41˜4.38 (m, 1H), 3.77 (s, 3H), 3.73˜3.59 (m, 5H), 3.24˜3.19 (m, 1H), 2.77-2.67 (m, 1H), 2.62˜2.56 (m, 1H), 2.44-2.41 (m, 1H), 1.99-1.91 (m, 1H), 1.79-1.74 (m, 1H), 1.49-1.44 (m, 2H), 1.35-1.32 (m, 3H). HPLC: 98.68% (220 nm), 98.70% (254 nm). MS (ESI): mass calcd. For C₃₀H₃₂F₂N₆O₃ 562.25 m/z found 563.3 [M+H]⁺.

### Compound 2

### 6-((((S)-1-(6-aminopyridin-3-yl)piperidin-3-yl)(pyridin-4-ylmethyl)amino)methyl)-9,10-difluoro-3-methyl-2H-[1,4]oxazino[2,3,4-ij]quinolin-7(3H)-one was Prepared According to the Procedure Described Herein for Step 8 in Scheme 1

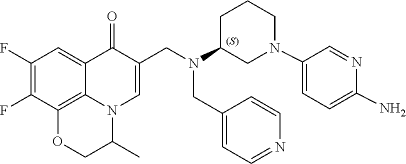

6-((((S)-1-(6-aminopyridin-3-yl)piperidin-3-yl)(pyridin-4-ylmethyl)amino)methyl)-9,10-difluoro-3-methyl-2H-[1,4]oxazino[2,3,4-ij]quinolin-7(3H)-one (27.2 mg, 50.80 umol, 16.81% yield, 99.46% purity) was obtained as yellow solid. ¹H NMR (DMSO-d₆, 400 MHz) δ 8.41 (d, J=6.0 Hz, 2H), 8.07 (d, J=6.4 Hz, 1H), 7.60 (d, J=2.8 Hz, 1H), 7.55-7.52 (m, 1H), 7.42˜7.41 (m, 2H), 7.16-7.12 (m, 1H), 6.36 (dd, J=3.6 Hz, 8.8 Hz, 1H), 5.36 (d, J=2.8 Hz, 2H), 4.71˜4.67 (m, 1H), 4.57-4.54 (m, 1H), 4.40 (d, J=9.2 Hz, 1H), 3.82˜3.77 (m, 2H), 3.68-3.62 (m, 1H), 3.61˜3.56 (m, 1H), 3.52 (d, J=10.0 Hz, 1H), 3.24-3.21 (m, 1H), 2.75-2.61 (m, 1H), 2.60-2.56 (m, 1H), 2.41 (s, 1H), 2.00-1.97 (m, 1H), 1.77-1.74 (m, 1H), 1.50-1.39 (m, 2H), 1.32 (dd, J=4.4, 6.4 Hz, 3H). MS: 99.46% (220 nm), 99.69% (254 nm). MS (ESI): mass calcd. For C₂₉H₃₀F₂N₆O₂ 532.24 m/z found 533.3 [M+H]⁺.

### Compound 3

### 6-((((S)-1-(6-aminopyridin-3-yl)piperidin-3-yl)((2-methylpyridin-4-yl)methyl)amino)methyl)-9,10-difluoro-3-methyl-2H-[1,4]oxazino[2,3,4-ij]quinolin-7(3H)-one was Prepared According to the Procedure Described Herein for Step 8 in Scheme 1

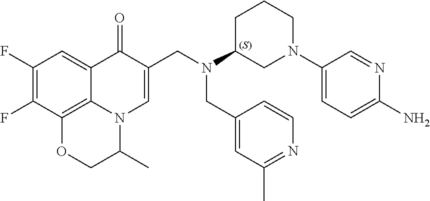

6-((((S)-1-(6-aminopyridin-3-yl)piperidin-3-yl)((2-methylpyridin-4-yl)methyl)amino)methyl)-9,10-difluoro-3-methyl-2H-[1,4]oxazino[2,3,4-ij]quinolin-7(3H)-one (28.9 mg, 52.62 umol, 17.85% yield, 99.53% purity) was obtained as white solid. ¹H NMR (DMSO-d₆, 400 MHz) δ 8.27 (d, J=4.8 Hz, 1H), 8.06 (d, J=4.4 Hz, 1H), 7.61 (s, 1H), 7.58-7.53 (m, 1H), 7.23˜7.20 (m, 2H), 7.14 (dd, J=2.4 Hz, 8.8 Hz, 1H), 6.37 (dd, J=2.8 Hz, 8.8 Hz, 1H), 5.37 (s, 2H), 4.71˜4.54 (m, 1H), 4.55 (dd, J=2.0 Hz, 11.2 Hz, 1H), 4.38 (d, J=10.8 Hz, 1H), 3.81˜3.55 (m, 4H), 3.52˜3.49 (m, 1H), 3.24˜3.22 (m, 1H), 2.78˜2.73 (m, 1H), 2.62˜2.57 (m, 1H), 2.46-2.39 (m, 1H), 2.36 (s, 3H), 2.02˜1.97 (m, 1H), 1.77-1.74 (m, 1H), 1.53-1.38 (m, 2H), 1.32 (dd, J=4.4 Hz, 6.8 Hz, 3H). HPLC: 99.53% (220 nm), 99.56% (254 nm). MS (ESI): mass calcd. For C₃₀H₃₂F₂N₆O₂ 546.26 m/z found 547.3 [M+H]⁺.

### Compound 4

### 6-((((S)-1-(6-aminopyridin-3-yl)piperidin-3-yl)((5-chloro-2-methoxypyridin-4-yl)methyl)amino)methyl)-9,10-difluoro-3-methyl-2H-[1,4]oxazino[2,3,4-ij]quinolin-7(3H)-one was Prepared According to the Procedure Described Herein for Step 8 in Scheme 1

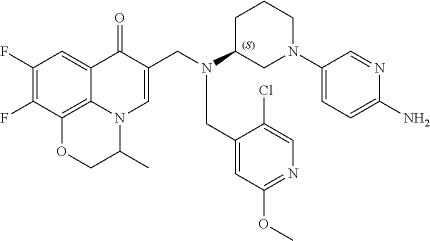

Compound 4 (17.4 mg, 29.09 umol, 18.24% yield, 99.81% purity) was obtained as pale pink solid. ¹H NMR (DMSO-d₆, 400 MHz) δ 8.08 (d, J=6.0 Hz, 1H), 8.04 (s, 1H), 7.62 (s, 1H), 7.56˜7.52 (m, 1H), 7.16˜7.14 (m, 1H), 7.10 (s, 1H), 6.38 (dd, J=3.6 Hz, 8.8 Hz, 1H), 5.37 (s, 2H), 4.62˜4.61 (m, 1H), 4.52 (d, J=11.2 Hz, 1H), 4.37˜4.35 (m, 1H), 3.83˜3.73 (m, 2H), 3.69 (s, 3H), 3.66-3.64 (m, 2H), 3.52 (d, J=10.0 Hz, 1H), 3.25-3.23 (m, 1H), 2.82˜2.80 (m, 1H), 2.61˜2.58 (m, 1H), 2.43˜2.40 (s, 1H), 2.00-1.97 (m, 1H), 1.76-1.75 (m, 1H), 1.47-1.44 (m, 2H), 1.30 (t, J=6.0 Hz, 3H). MS: 99.81% (220 nm), 100.00% (254 nm). MS (ESI): mass calcd. For C₃₀H₃₁ClF₂N₆O₃ 596.21 m/z found 597.2 [M+H]⁺.

### Compound 5 and Compound 6

### Preparation of (R)-6-((((S)-1-(6-aminopyridin-3-yl)piperidin-3-yl)((2-methoxypyridin-4-yl)methyl)amino)methyl)-9,10-difluoro-3-methyl-2H-[1,4]oxazino[2,3,4-ij]quinolin-7(3H)-one and (S)-6-((((S)-1-(6-aminopyridin-3-yl)piperidin-3-yl)((2-methoxypyridin-4-yl)methyl)amino)methyl)-9,10-difluoro-3-methyl-2H-[1,4]oxazino[2,3,4-ij]quinolin-7(3H)-one (Step 9 in Scheme 1)

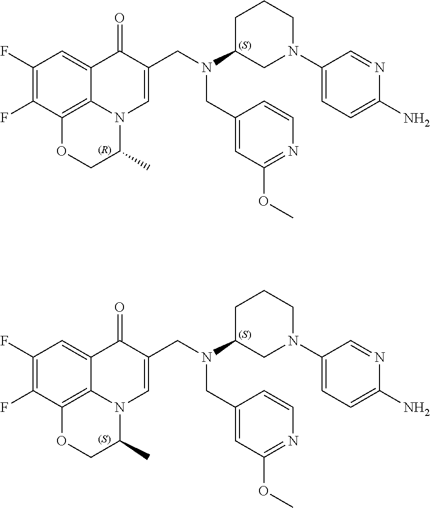

11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (40 mg, 71.10 umol, 1 eq) was separated by SFC (column: DAICEL CHIRALCEL OD (250 mm*30 mm, 10 um); mobile phase: [0.1% NH₃H₂O EtOH]; B %: 50%-50%, 17 mins). The eluent was concentrated under reduced pressure. The obtained was separately dissolved in MeCN (5 mL) and water (20 mL), then dried under freeze drying. Compound (2R)-11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (14.3 mg, 25.42 umol, 35.75% yield, 100% purity) was obtained as white solid. ¹H NMR (DMSO-d₆, 400 MHz) δ 8.07 (s, 1H), 7.99 (d, J=5.2 Hz, 1H), 7.61 (s, 1H), 7.56-7.52 (m, 1H), 7.16-7.13 (m, 1H), 7.01˜6.99 (m, 1H), 6.82 (s, 1H), 6.38˜6.36 (m, 1H), 5.36 (s, 2H), 4.71˜4.69 (m, 1H), 4.56˜4.54 (m, 1H), 4.40˜4.37 (m, 1H), 3.76 (s, 3H), 3.73˜3.59 (m, 5H), 3.24˜3.19 (m, 1H), 2.70˜2.67 (m, 1H), 2.70-2.56 (m, 1H), 2.48-2.43 (m, 1H), 1.99-1.91 (m, 1H), 1.74-1.71 (m, 1H), 1.49-1.44 (m, 2H), 1.36-1.33 (m, 3H). HPLC: 100% (220 nm), 100% (254 nm). MS (ESI): mass calcd. For C₃₀H₃₂F₂N₆O₃ 562.25 m/z found 563.3 [M+H]⁺. Compound (2S)-11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (11.4 mg, 20.26 umol, 28.50% yield, 100% purity) was obtained as white solid. The structure was assigned randomly. ¹H NMR (DMSO-d₆, 400 MHz) δ 8.09 (s, 1H), 7.98 (d, J=5.2 Hz, 1H), 7.61 (s, 1H), 7.56-7.52 (m, 1H), 7.16-7.13 (m, 1H), 7.01˜6.99 (m, 1H), 6.81 (s, 1H), 6.39-6.36 (m, 1H), 5.37 (s, 2H), 4.70˜4.68 (m, 1H), 4.57˜4.54 (m, 1H), 4.40˜4.37 (m, 1H), 3.76 (s, 3H), 3.73˜3.59 (m, 5H), 3.24˜3.19 (m, 1H), 2.70˜2.67 (m, 1H), 2.70˜2.56 (m, 1H), 2.48˜2.43 (m, 1H), 1.99˜1.91 (m, 1H), 1.74˜1.71 (m, 1H), 1.49-1.44 (m, 2H), 1.33˜1.31 (m, 3H). HPLC: 100% (220 nm), 100% (254 nm). MS (ESI): mass calcd. For C₃₀H₃₂F₂N₆O₃ 562.25 m/z found 563.3 [M+H]⁺.

### Compound 7

### Preparation of N-(5-((3S)-3-(((9,10-difluoro-3-methyl-7-oxo-3,7-dihydro-2H-[1,4]oxazino[2,3,4-ij]quinolin-6-yl)methyl)((2-methoxypyridin-4-yl)methyl)amino)piperidin-1-yl)pyridin-2-yl)acetamide (Step 10 in Scheme 1)

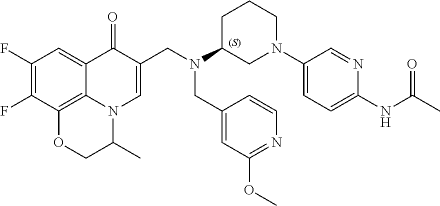

To a mixture of 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (30 mg, 53.32 umol, 1 eq) and DIPEA (41.35 mg, 319.94 umol, 55.73 uL, 6 eq) in DMF (1 mL) was added Ac₂O (6.53 mg, 63.99 umol, 5.99 uL, 1.2 eq) dropwise at 0° C. The reaction mixture was stirred at 20° C. for 2 hours under N₂. Then the reaction mixture was stirred at 20° C. for 10 hours under N₂. LCMS and HPLC showed the reaction was complete. The solution was directly purified by prep-HPLC (column: Waters Xbridge BEH C18 100*30 mm*10 um; mobile phase: [water (NH₄HCO₃)-MeCN]; B %: 30%-60%, 8 mins). The eluent was removed under freeze drying. Compound N-[5-[(3S)-3-[(6,7-difluoro-2-methyl-10-oxo-4-oxa-1-azatricyclo [7.3.1.0^{5,13}]trideca-5(13),6,8,11-tetraen-11-yl)methyl-[(2-methoxy-4-pyridyl)methyl]amino]-1-piperidyl]-2-pyridyl]acetamide (9.5 mg, 15.60 umol, 29.25% yield, 99.26% purity) was obtained as white solid. ¹H NMR (DMSO-d₆, 400 MHz) δ 10.17 (s, 1H), 8.10 (d, J=6.4 Hz, 1H), 8.00-7.97 (m, 2H), 7.87 (t, J=8.4 Hz, 1H), 7.54-7.59 (m, 1H), 7.35 (d, J=9.2 Hz, 1H), 7.02 (d, J=4.8 Hz, 1H), 6.84 (d, J=4.8 Hz, 1H), 4.79-4.73 (m, 1H), 4.56 (d, J=10.0 Hz, 1H), 4.40 (d, J=4.8 Hz, 1H), 3.80-3.55 (m, 8H), 3.31˜3.28 (m, 1H), 2.74˜2.52 (m, 3H), 2.03 (s, 3H), 2.02˜1.99 (m, 1H), 1.77˜1.73 (m, 1H), 1.54-1.43 (m, 2H), 1.35-1.31 (m, 3H). HPLC: 99.26% (220 nm), 99.32% (254 nm). MS (ESI): mass calcd. For C₃₂H₃₄F₂N₆O₄ 604.26 m/z found 605.3 [M+H]⁺.

### Compound 8

### Preparation of (E)-4-(2-ethoxyvinyl)-2-methoxypyridine (Step 11 in Scheme 1)

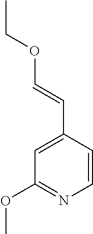

A mixture of 4-bromo-2-methoxy-pyridine (500 mg, 2.66 mmol, 1 eq), 2-[(E)-2-ethoxyvinyl]-4,4,5,5-tetramethyl-1,3,2-dioxaborolane (632.05 mg, 3.19 mmol, 1.2 eq), K₂CO₃ (1.10 g, 7.98 mmol, 3 eq) and Pd(dppf)Cl₂ (291.87 mg, 398.89 umol, 0.15 eq) in dioxane (6 mL) and H₂O (0.6 mL) was degassed and purged with N₂ for 3 times. Then the mixture was stirred at 100° C. for 16 hours under N₂ atmosphere. LCMS indicated the reaction was complete. The reaction mixture was cooled to room temperature, filtered through a pad of the Celite and the filter cake was washed with EtOAc (30 mL). The combined filtrate was concentrated under reduced pressure. The residue was purified by flash silica gel chromatography (Biotage®; 40 g SepaFlash® Silica Flash Column, Eluent of 0-20% Ethyl acetate/Petroleum ether gradient @75 mL/min). The eluent was concentrated in vacuum. Compound 4-[(E)-2-ethoxyvinyl]-2-methoxy-pyridine (400 mg, 2.23 mmol, 83.93% yield) was obtained as colorless oil. ¹H NMR (DMSO-d₆, 400 MHz) δ 7.95 (d, J=5.6 Hz, 1H), 7.52 (d, J=13.2 Hz, 1H), 6.92 (dd, J=5.2 Hz, 1.2 Hz, 1H), 6.65 (s, 1H), 5.76 (d, J=12.8 Hz, 1H), 3.94 (q, J=6.8 Hz, 2H), 3.80 (s, 3H), 1.25 (t, J=7.2 Hz, 3H).

### Preparation of 2-(2-methoxypyridin-4-yl)acetaldehyde (Step 12 in Scheme 1)

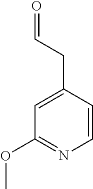

To a solution of 4-[(E)-2-ethoxyvinyl]-2-methoxy-pyridine (120 mg, 669.59 umol, 1 eq) in DCM (1 mL) was added TFA (3.23 g, 28.36 mmol, 2.10 mL, 42.36 eq). The mixture was stirred at 50° C. for 16 hours. LCMS indicated 4-[(E)-2-ethoxyvinyl]-2-methoxy-pyridine was consumed completely and one new peak with desired mass was detected. The reaction mixture was quenched by addition H₂O (1 mL) at 0° C. and then adjusted to pH=7 with sat. Na₂CO₃. The mixture was extracted with EtOAc (20 mL×3). The combined organic layers were washed with brine (10 mL×1), dried over anhydrous Na₂SO₄, filtered and concentrated under reduced pressure. Compound 2-(2-methoxy-4-pyridyl)acetaldehyde (100 mg, crude) was obtained as colorless oil. ¹H NMR (DMSO-d₆, 400 MHz) δ 9.68 (s, 1H), 8.28 (d, J=5.2 Hz, 1H), 7.39 (dd, J=5.2 Hz, 1.6 Hz, 1H), 7.22 (s, 1H), 3.90 (s, 3H), 3.84 (s, 2H).

### Preparation of 9,10-difluoro-6-(((2-(2-methoxypyridin-4-yl)ethyl)((S)-1-(6-nitropyridin-3-yl)piperidin-3-yl)amino)methyl)-3-methyl-2H-[1,4]oxazino[2,3,4-ij]quinolin-7(3H)-one (Step 13 in Scheme 1)

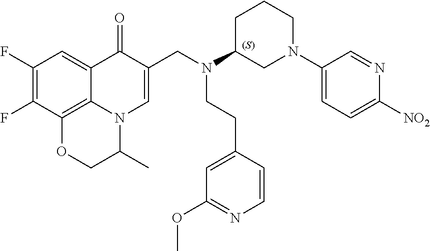

To a mixture of 6,7-difluoro-2-methyl-11-[[[(3S)-1-(6-nitro-3-pyridyl)-3-piperidyl]amino]methyl]-4-oxa-1-azatricyclo[7.3.1.0^{5,13}]trideca-5(13),6,8,11-tetraen-10-one (100 mg, 212.11 umol, 1 eq) and 2-(2-methoxy-4-pyridyl)acetaldehyde (100 mg, 661.54 umol, 3.12 eq) in DCE (3 mL) was added NaBH(OAc)₃ (269.73 mg, 1.27 mmol, 6 eq). The reaction mixture was stirred at 20° C. for 2 hours. LCMS indicated 6,7-difluoro-2-methyl-11-[[[(3S)-1-(6-nitro-3-pyridyl)-3-piperidyl]amino]methyl]-4-oxa-1-azatricyclo[7.3.1.0^{5,13}]trideca-5(13),6,8,11-tetraen-10-one was remained, several new peaks were formed and one new peak with desired mass was detected. The reaction mixture was quenched by addition H₂O (2 mL) at 0° C. and then extracted with DCM (5 mL×3). The combined organic layers were washed with brine (3 mL×2), dried over anhydrous Na₂SO₄, filtered and concentrated under reduced pressure. The residue was purified by flash silica gel chromatography (Biotage®; 12 g SepaFlash® Silica Flash Column, Eluent of 0-80% Ethyl acetate/Petroleum ether gradient @40 mL/min). The eluent was concentrated in vacuum. Compound 6,7-difluoro-11-[[2-(2-methoxy-4-pyridyl) ethyl-[(3S)-1-(6-nitro-3-pyridyl)-3-piperidyl]amino]methyl]-2-methyl-4-oxa-1-azatricyclo[7.3.1.0^{5,13}]trideca-5(13),6,8,11-tetraen-10-one (70 mg, crude) was obtained as yellow solid. ¹H NMR (DMSO-d₆, 400 MHz) δ 8.18 (d, J=2.4 Hz, 1H), 8.11˜8.08 (m, 1H), 7.99 (d, J=5.2 Hz, 1H), 7.87 (d, J=10.0 Hz, 1H), 7.59-7.53 (m, 1H), 7.43˜7.37 (m, 1H), 6.83 (d, J=5.2 Hz, 1H), 6.64 (s, 1H), 4.59-4.53 (m, 2H), 4.43˜4.41 (m, 1H), 4.05˜4.02 (m, 3H), 3.82˜3.80 (m, 2H), 3.32˜3.28 (m, 2H), 3.09-2.94 (m, 2H), 2.91˜2.87 (m, 1H), 2.70-2.67 (m, 2H), 2.40-2.32 (m, 1H), 2.19-2.15 (m, 1H), 1.90˜1.86 (m, 1H), 1.78˜1.76 (m, 1H), 1.64˜1.56 (m, 1H), 1.49˜1.47 (m, 1H), 1.38˜1.29 (m, 3H).

### Preparation of 6-((((S)-1-(6-aminopyridin-3-yl)piperidin-3-yl)(2-(2-methoxypyridin-4-yl)ethyl)amino)methyl)-9,10-difluoro-3-methyl-2H-[1,4]oxazino[2,3,4-ij]quinolin-7(3H)-one (Step 14 in Scheme 1)

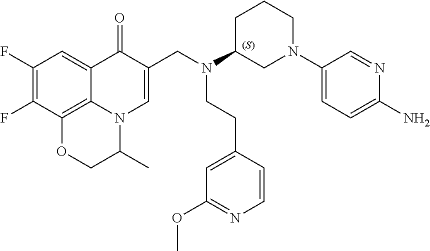

To a solution of 6,7-difluoro-11-[[2-(2-methoxy-4-pyridyl)ethyl-[(3S)-1-(6-nitro-3-pyridyl)-3-piperidyl]amino]methyl]-2-methyl-4-oxa-1-azatricyclo[7.3.1.0^{5,13}]trideca-5(13),6,8,11-tetraen-10-one (70 mg, 115.39 umol, 1 eq) in EtOH (3 mL) and H₂O (0.4 mL) was added Fe (32.22 mg, 576.97 umol, 5 eq) and NH₄Cl (12.34 mg, 230.79 umol, 2 eq). The mixture was stirred at 65° C. for 3 hours. LCMS and HPLC indicated 6,7-difluoro-11-[[2-(2-methoxy-4-pyridyl)ethyl-[(3S)-1-(6-nitro-3-pyridyl)-3-piperidyl]amino]methyl]-2-methyl-4-oxa-1-azatricyclo[7.3.1.0^{5,13}]trideca-5(13),6,8,11-tetraen-10-one was consumed completely and major one peak with desired mass was detected. The reaction mixture was cooled to room temperature and filtered through a pad of the Celite. The filtrate was concentrated in vacuum. The residue was purified by prep-HPLC (column: Waters Xbridge BEH C18 100*30 mm*10 um; mobile phase: [water (NH₄HCO₃)-MeCN]; B %: 40%-60%, 8 mins). The eluent was dried over lyophilization. Compound 11-[[[(3S)-1-(6-amino-3-pyridyl)-3-piperidyl]-[2-(2-methoxy-4-pyridyl)ethyl]amino]methyl]-6,7-difluoro-2-methyl-4-oxa-1-azatricyclo[7.3.1.0^{5,13}]trideca-5(13),6,8,11-tetraen-10-one (10.9 mg, 17.84 umol, 15.46% yield, 94.04% purity) was obtained as pale pink solid. ¹H NMR (DMSO-d₆, 400 MHz) δ 7.98 (d, J=5.2 Hz, 1H), 7.83 (d, J=4.4 Hz, 1H), 7.60-7.54 (m, 2H), 7.11˜7.07 (m, 1H), 6.83 (d, J=5.2 Hz, 1H), 6.63 (s, 1H), 6.36 (d, J=8.8 Hz, 1H), 5.35 (s, 2H), 4.57˜4.51 (m, 2H), 4.42 (d, J=7.6 Hz, 1H), 3.77 (s, 3H), 3.61 (s, 2H), 3.26˜3.20 (m, 2H), 2.85˜2.80 (m, 2H), 2.78˜2.73 (m, 1H), 2.70˜2.67 (m, 2H), 2.44˜2.38 (m, 2H), 1.85˜1.82 (m, 1H), 1.74˜1.71 (m, 1H), 1.56˜1.48 (m, 1H), 1.34˜1.31 (m, 4H). LCMS: 94.04% (220 nm), 94.13% (254 nm). MS (ESI): mass calcd. For C₃₁H₃₄F₂N₆O₃ 576.27 m/z found 577.4 [M+H]⁺.

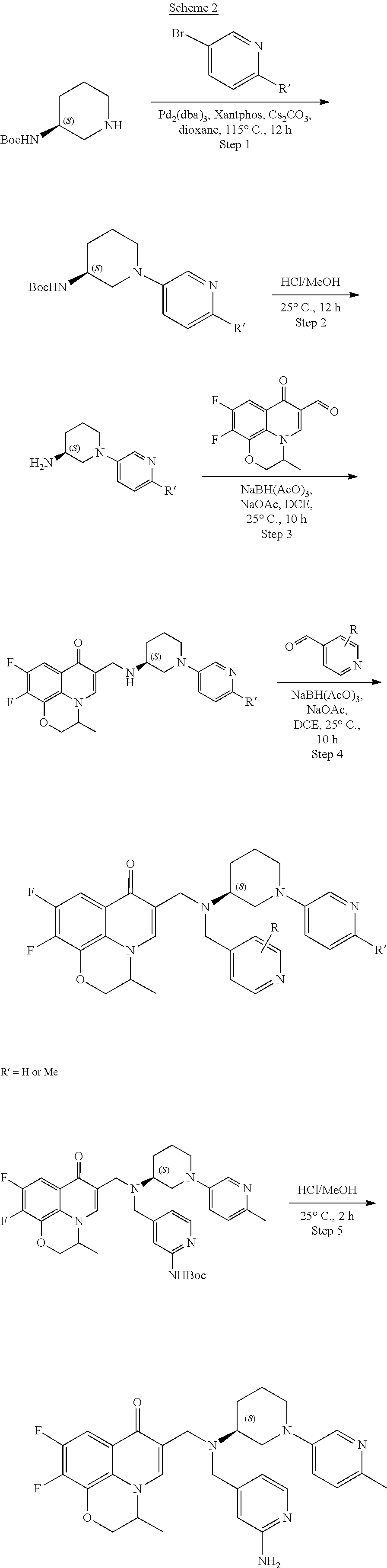

### General Procedures for Preparing Compounds in Scheme 2

### Preparation of Compounds in Scheme 2 (Step 1 in Scheme 2)

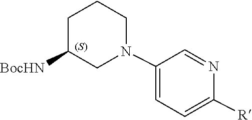

A mixture of tert-butyl N-(3-piperidyl)carbamate (22.67 mmol, 1.3 eq), 5-bromo-2-methyl (or H)-pyridine (17.44 mmol, 1 eq), Pd₂(dba)₃ (871.98 umol, 0.05 eq), Xantphos (871.98 umol, 0.05 eq) and Cs₂CO₃ (23.54 mmol, 1.35 eq) in dioxane (3 mL/mmol˜4 mL/mmol) was degassed and purged with N₂ for 3 times. Then the reaction mixture was stirred at 100° C. 115° C. for 10 hours˜12 hours under N₂ atmosphere. LCMS showed the reaction was complete. The reaction mixture was cooled to room temperature, filtered through a pad of the Celite. The filtrate was concentrated under reduced pressure. The residue was purified by flash silica gel chromatography to give the desired product.

### Preparation of Compounds in Scheme 2 (Step 2 in Scheme 2)

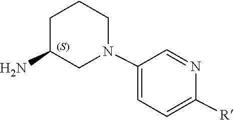

To a solution of tert-butyl N-[1-(6-methyl (or H)-3-pyridyl)-3-piperidyl]carbamate (3.43 mmol, 1 eq) in EtOAc (3 mL/mmol) or MeOH (3 mL/mmol) was added HCl/EtOAc (4 M, 6 mL/mmol) or HCl/MeOH (4 M, 4 mL/mmol). The reaction mixture was stirred at 20° C. for 12 hours. LC-MS showed the reaction was complete. The mixture was concentrated under reduced pressure to obtain the desired product.

### Preparation of Compounds in Scheme 2 (Step 3 in Scheme 2)

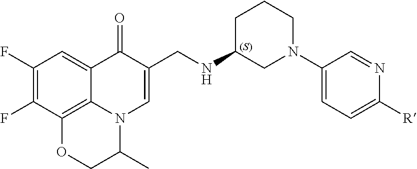

To a mixture of (3S)-1-(6-methyl (or H)-3-pyridyl)piperidin-3-amine (2.00 mmol, 1 eq, 3HCl) and 6,7-difluoro-2-methyl-10-oxo-4-oxa-1-azatricyclo[7.3.1.05,13]trideca-5(13),6,8,11-tetraene-11-carbaldehyde (2.00 mmol, 1 eq) in DCE (7.0 mL/mmol˜7.5 mL/mmol) was added NaOAc (2.20 mmol, 1.1 eq) at 20° C. under N₂. The mixture was stirred at 20° C. for half an hour˜5 hours and then NaBH(OAc)₃ (2.99 mmol˜3.99 mmol, 1.5 eq˜2.0 eq) was added at 0° C. The mixture was stirred at 20° C. for 2 hours˜6 hours. The reaction mixture was quenched by addition water at 0° C. and then extracted with EtOAc. The combined organic layers were washed with brine, dried over Na₂SO₄, filtered and concentrated under reduced pressure to give the desired product.

### Preparation of Compounds in Scheme 2 (Step 4 in Scheme 2)

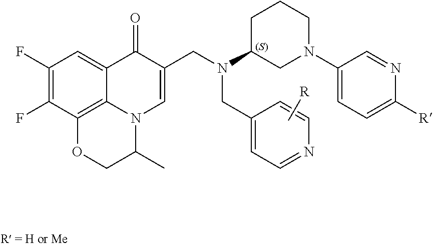

To a mixture of 6,7-difluoro-2-methyl-11-[[[(3S)-1-(6-methyl (or H)-3-pyridyl)-3-piperidyl]amino]methyl]-4-oxa-1-azatricyclo[7.3.1.05,13]trideca-5(13),6,8,11-tetraen-10-one (204.32 umol, 1 eq) and different aldehyde (224.75 umol, 1.1 eq) in DCE (6.5 mL/mmol˜14.7 mL/mmol) was added NaBH(OAc)₃ (306.48 umol, 1.5 eq) at 0° C. under N₂. Then the mixture was stirred at 20° C. for 10 hours˜20 hours. The reaction mixture was quenched with ice water at 0° C. and made pH=8 with sat. NaHCO₃. The mixture was extracted with DCM and i-PrOH (v:v=3:1). The combined organic layer was washed with brine (20 mL×1), dried over Na₂SO₄, filtered and concentrated under reduced pressure. The residue was purified by prep-HPLC (column: Phenomenex C18 75*30 mm*3 um or Waters Xbridge Prep OBD C 18 150*40 mm*10 um; mobile phase: [water (NH₄HCO₃)-MeCN]; B %: 25%-60%, 8 mins). The solvent was removed under freeze drying to give the desired product.

### Preparation of (S)-tert-butyl (1-(6-methylpyridin-3-yl)piperidin-3-yl)carbamate in Scheme 2 (Step 1 in Scheme 2)

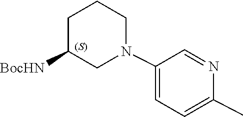

A mixture of tert-butyl N-(3-piperidyl)carbamate (4.54 g, 22.67 mmol, 1.3 eq), 5-bromo-2-methyl-pyridine (3 g, 17.44 mmol, 1 eq), Pd₂(dba)₃ (798.49 mg, 871.98 umol, 0.05 eq), Xantphos (504.54 mg, 871.98 umol, 0.05 eq) and Cs₂CO₃ (7.67 g, 23.54 mmol, 1.35 eq) in dioxane (50 mL) was degassed and purged with N₂ for 3 times, and then the mixture was stirred at 115° C. for 12 hours under N₂ atmosphere. LCMS showed the reaction was complete. The reaction mixture was cooled to room temperature, filtered through a pad of the Celite. The filtrate was concentrated under reduced pressure. The residue was purified by flash silica gel chromatography (Biotage; 40 SepaFlash® Silica Flash Column, Eluent of 0˜60% Ethyl acetate/Petroleum ether gradient @60 mL/min). The eluent was removed under reduced pressure. Compound tert-butyl N-[1-(6-methyl-3-pyridyl)-3-piperidyl]carbamate (1 g, 3.43 mmol, 19.68% yield) was obtained as yellow solid. ¹H NMR (400 MHz, DMSO-d₆) δ 8.12 (s, 1H), 7.22˜7.19 (m, 1H), 7.05 (d, J=8.8 Hz, 1H), 6.88 (d, J=7.2 Hz, 1H), 3.57-3.49 (m, 3H), 2.67-2.61 (m, 1H), 2.53 (s, 1H), 2.33 (s, 3H), 1.81˜1.70 (m, 2H), 1.53˜1.50 (m, 1H), 1.40 (s, 9H), 1.36˜1.32 (m, 1H).

### Preparation of (S)-1-(6-methylpyridin-3-yl)piperidin-3-amine in Scheme 2 (Step 2 in Scheme 2)

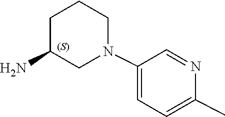

To a solution of tert-butyl N-[1-(6-methyl-3-pyridyl)-3-piperidyl]carbamate (1 g, 3.43 mmol, 1 eq) in EtOAc (10 mL) as added HCl/EtOAc (4 M, 56 mL, 67 eq). The mixture was stirred at 20° C. for 12 hours. LC-MS showed the reaction was complete. The mixture was concentrated under reduced pressure. Compound (3S)-1-(6-methyl-3-pyridyl)piperidin-3-amine (1 g, 3.33 mmol, 96.92% yield, 3HCl) was obtained as brown solid. ¹H NMR (400 MHz, DMSO-d₆) δ 8.44 (s, 3H), 8.29 (s, 1H), 8.07 (dd, J=2.8 Hz, J=8.8 Hz, 1H), 7.72 (d, J=9.2 Hz, 1H), 3.88˜3.81 (m, 1H), 3.63˜3.59 (m, 1H), 3.34˜3.26 (m, 2H), 3.16˜3.14 (m, 1H), 2.60 (s, 3H), 1.91˜1.83 (m, 1H), 1.82˜1.78 (m, 1H), 1.70˜1.65 (m, 1H), 1.60˜1.57 (m, 1H).

### Preparation of 9,10-difluoro-3-methyl-6-((((S)-1-(6-methylpyridin-3-yl)piperidin-3-yl)amino)methyl)-2H-[1,4]oxazino[2,3,4-ij]quinolin-7(3H)-one in Scheme 2 (Step 3 in Scheme 2)

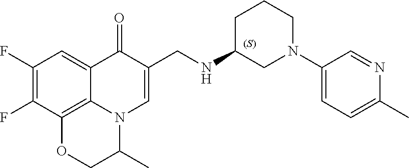

To a mixture of (3S)-1-(6-methyl-3-pyridyl)piperidin-3-amine (600 mg, 2.00 mmol, 1 eq, 3HCl) and 6,7-difluoro-2-methyl-10-oxo-4-oxa-1-azatricyclo[7.3.1.05,13]trideca-5(13),6,8, 11-tetraene-11-carbaldehyde (529.27 mg, 2.00 mmol, 1 eq) in DCE (15 mL) was added NaOAc (180.08 mg, 2.20 mmol, 1.1 eq) at 20° C. under N₂. The mixture was stirred at 20° C. for 5 hours and then NaBH(OAc)₃ (634.44 mg, 2.99 mmol, 1.5 eq) was added at 0° C. The mixture was stirred at 20° C. for 2 hours. LCMS showed the reaction was complete. The reaction mixture was quenched by addition water (30 mL) at 0° C. and then extracted with EtOAc (30 mL×3). The combined organic layers were washed with brine (20 mL×2), dried over Na₂SO₄, filtered and concentrated under reduced pressure. Compound 6,7-difluoro-2-methyl-11-[[[(3S)-1-(6-methyl-3-pyridyl)-3-piperidyl]amino]methyl]-4-oxa-1-azatricyclo[7.3.1.05,13]trideca-5(13),6,8,11-tetraen-10-one (800 mg, crude) was obtained as brown solid. ¹H NMR (400 MHz, DMSO-d₆) δ 8.12 (s, 1H), 7.97 (d, J=5.2 Hz, 1H), 7.60-7.55 (m, 1H), 7.21 (d, J=7.2 Hz, 1H), 7.03 (d, J=8.8 Hz, 1H), 4.65-4.55 (m, 3H), 4.42˜4.38 (m, 2H), 3.68-3.62 (m, 2H), 3.47-3.44 (m, 1H), 2.72˜2.64 (m, 2H), 2.33 (s, 3H), 1.91˜1.80 (m, 1H), 1.74˜1.72 (m, 1H), 1.54˜1.51 (m, 2H), 1.40 (s, 3H).

### Compound 9

### Preparation of 9,10-difluoro-3-methyl-6-((((S)-1-(6-methylpyridin-3-yl)piperidin-3-yl)((2-methylpyridin-4-yl)methyl)amino)methyl)-2H-[1,4]oxazino[2,3,4-ij]quinolin-7(3H)-one in Scheme 2 (Step 4 in Scheme 2)

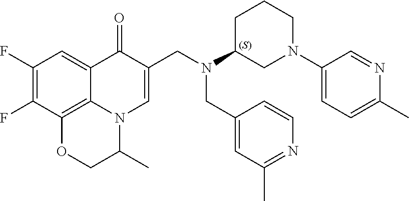

To a mixture of 6,7-difluoro-2-methyl-11-[[[(3S)-1-(6-methyl-3-pyridyl)-3-piperidyl]amino]methyl]-4-oxa-1-azatricyclo[7.3.1.05,13]trideca-5(13),6,8,11-tetraen-10-one (90 mg, 204.32 umol, 1 eq) and 2-methylpyridine-4-carbaldehyde (27.23 mg, 224.75 umol, 1.1 eq) in DCE (1.5 mL) was added NaBH(OAc)₃ (64.96 mg, 306.48 umol, 1.5 eq) at 0° C. under N₂. Then the mixture was stirred at 20° C. for 10 hours. LCMS showed 6,7-difluoro-2-methyl-11-[[[(3S)-1-(6-methyl-3-pyridyl)-3-piperidyl]amino]methyl]-4-oxa-1-azatricyclo[7.3.1.05,13]trideca-5(13),6,8,11-tetraen-10-one was consumed completely and one major peak with desired mass was detected. The reaction mixture was quenched by addition water (1 mL) at 0° C. and then the mixture concentrated under reduced pressure. The residue was purified by prep-HPLC (column: Phenomenex C18 75*30 mm*3 um; mobile phase: [water (NH₄HCO₃)-MeCN]; B %: 30%-60%, 8 mins). The solvent was removed under freeze drying. Compound 6,7-difluoro-2-methyl-11-[[(2-methyl-4-pyridyl)methyl-[(3S)-1-(6-methyl-3-pyridyl)-3-piperidyl]amino]methyl]-4-oxa-1-azatricyclo[7.3.1.05,13]trideca-5(13),6,8,11-tetraen-10-one (19.5 mg, 34.81 umol, 17.04% yield, 97.39% purity) was obtained as white solid. ¹H NMR (400 MHz, DMSO-d₆) δ 8.28 (d, J=4.8 Hz, 1H), 8.15 (s, 1H), 8.04 (s, 1H), 7.57˜7.55 (m, 1H), 7.24˜7.19 (m, 3H), 7.01 (t, J=8.0 Hz, 1H), 4.71˜4.70 (m, 1H), 4.56-4.54 (m, 1H), 4.39 (d, J=9.6 Hz, 1H), 3.84-3.80 (m, 1H), 3.74-3.71 (m, 2H), 3.71˜3.64 (m, 2H), 3.61˜3.57 (m, 1H), 2.79-2.76 (m, 2H), 2.75-2.71 (m, 1H), 2.37 (s, 3H), 2.32 (s, 3H), 1.99˜1.95 (m, 1H), 1.77˜1.74 (m, 1H), 1.50˜1.48 (m, 2H), 1.32 (t, J=7.2 Hz, 3H). HPLC: 97.39% (220 nm), 97.92% (254 nm). MS (ESI): mass calcd. For C₃₁H₃₃F₂N₅O₂ 545.26 m/z found 546.3 [M+H]⁺.

### Compound 10

### 9,10-difluoro-3-methyl-6-((((2-methylpyridin-4-yl)methyl)((S)-1-(pyridin-3-yl)piperidin-3-yl)amino)methyl)-2H-[1,4]oxazino[2,3,4-ij]quinolin-7(3H)-one was Prepared According to the Procedure Described Herein for Step 4 in Scheme 2

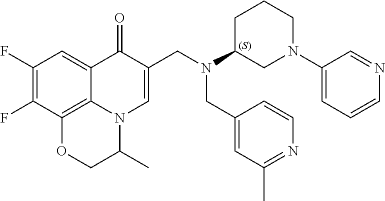

9,10-difluoro-3-methyl-6-((((2-methylpyridin-4-yl)methyl)((S)-1-(pyridin-3-yl)piperidin-3-yl)amino)methyl)-2H-[1,4]oxazino[2,3,4-ij]quinolin-7(3H)-one (14 mg, 26.14 umol, 11.15% yield, 99.24% purity) was obtained as white solid. ¹H NMR (400 MHz, DMSO-d₆) δ 8.30-8.27 (m, 2H), 8.10 (d, J=4.4 Hz, 1H), 7.92 (t, J=4.0 Hz, 1H), 7.58-7.52 (m, 1H), 7.30-7.22 (m, 3H), 7.17-7.13 (m, 1H), 4.71˜4.68 (m, 1H), 4.59-4.56 (m, 1H), 4.40-4.31 (m, 1H), 3.97˜3.93 (m, 1H), 3.72˜3.57 (m, 5H), 2.85˜2.82 (m, 1H), 2.73˜2.63 (m, 2H), 2.38 (s, 3H), 2.01˜1.96 (m, 1H), 1.79-1.74 (m, 1H), 1.60-1.38 (m, 2H), 1.32 (d, J=6.8 Hz, 8.8 Hz, 3H). HPLC: 99.24% (220 nm), 98.93% (254 nm). MS (ESI): mass calcd. For C₃₀H₃₁F₂N₅O₂ 531.24 m/z found 532.3 [M+H]⁺.

### Compound 11

### Preparation of 6-((((2-aminopyridin-4-yl)methyl)((S)-1-(6-methylpyridin-3-yl)piperidin-3-yl)amino)methyl)-9,10-difluoro-3-methyl-2H-[1,4]oxazino[2,3,4-ij]quinolin-7(3H)-one (Step 5 in Scheme 2)

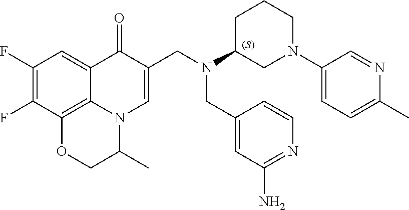

A solution of tert-butyl N-[4-[[(6,7-difluoro-2-methyl-10-oxo-4-oxa-1-azatricyclo [7.3.1.05,13]trideca-5(13),6,8,11-tetraen-11-yl)methyl-[(3S)-1-(6-methyl-3-pyridyl)-3-piperidyl]amino]methyl]-2-pyridyl]carbamate (150 mg, 231.94 umol, 1 eq) in HCl/MeOH (4 M, 45.00 mL, 776.07 eq) was stirred at 20° C. for 2 hours. LC-MS showed the reaction was complete. The reaction mixture was concentrated under reduced pressure. The residue was purified by prep-HPLC (column: Phenomenex C 18 80*40 mm*3 um; mobile phase: [water (NH₄HCO₃)-MeCN]; B %: 20%-50%, 8 mins). The solvent was removed under freeze drying. Compound 11-[[(2-amino-4-pyridyl)methyl-[(3S)-1-(6-methyl-3-pyridyl)-3-piperidyl]amino]methyl]-6,7-difluoro-2-methyl-4-oxa-1-azatricyclo[7.3.1.05,13]trideca-5(13),6,8,11-tetraen-10-one (16.6 mg, 28.26 umol, 12.18% yield, 93.06% purity) was obtained as white solid. ¹H NMR (400 MHz, DMSO-d₆) δ 8.13 (s, 1H), 8.03 (d, J=6.0 Hz, 1H), 7.76 (d, J=5.6 Hz, 1H), 7.58-7.53 (m, 1H), 7.20 (dd, J=3.2 Hz, 7.6 Hz, 1H), 7.02 (t, J=7.6 Hz, 1H), 6.55 (t, J=4.8 Hz, 1H), 6.45 (s, 1H), 5.75 (s, 2H), 4.69-4.66 (m, 1H), 4.57 (d, J=4 Hz, 1H), 4.43˜4.40 (m, 1H), 3.79 (d, J=8.8 Hz, 1H), 3.69˜3.53 (m, 5H), 2.76˜2.67 (m, 2H), 2.63˜2.56 (m, 1H), 2.32 (s, 3H), 2.00˜1.97 (m, 1H), 1.78-1.75 (m, 1H), 1.54-1.44 (m, 2H), 1.35 (t, J=6.4 Hz, 3H). HPLC: 93.06% (220 nm), 98.18% (254 nm). MS (ESI): mass calcd. For C₃₀H₃₂F₂N₆O₂ 546.26 m/z found 547.3 [M+H]⁺.

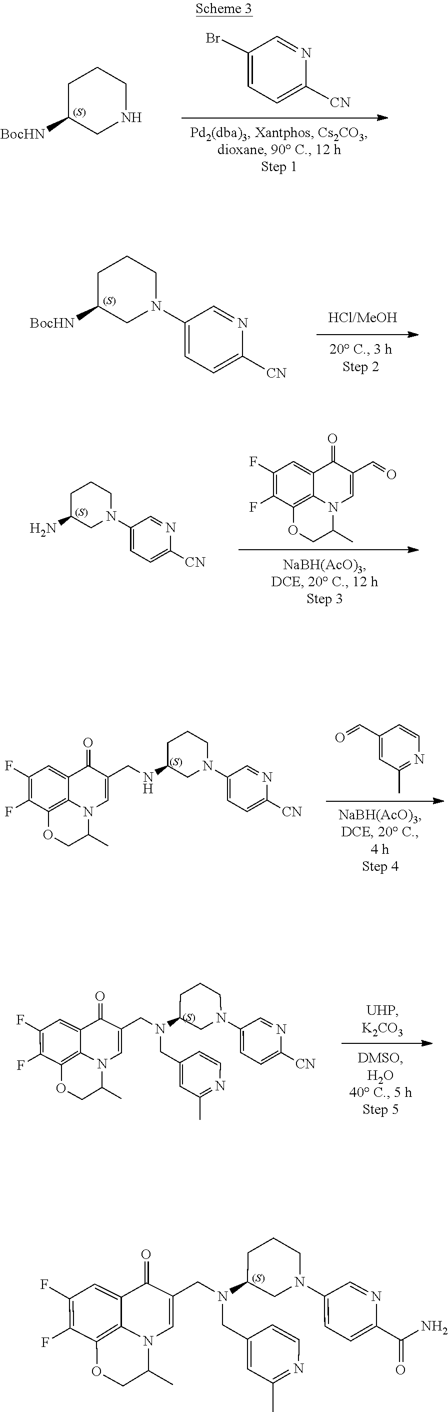

### Specific Procedures for Preparing Compounds in Scheme 3

### Preparation of (S)-tert-butyl (1-(6-cyanopyridin-3-yl)piperidin-3-yl)carbamate (Step 1 in Scheme 3)

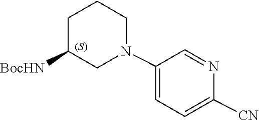

A mixture of tert-butyl N-(3-piperidyl)carbamate (1.42 g, 7.10 mmol, 1.3 eq), 5-bromopyridine-2-carbonitrile (1 g, 5.46 mmol, 1 eq), Pd₂(dba)₃ (250.19 mg, 273.22 umol, 0.05 eq), Xantphos (189.71 mg, 327.86 umol, 0.06 eq) and Cs₂CO₃ (2.40 g, 7.38 mmol, 1.35 eq) in dioxane (20 mL) was degassed and purged with N₂ for 3 times and then the mixture was stirred at 90° C. for 12 hours under N₂ atmosphere. LCMS showed the reaction was complete. The reaction mixture was concentrated under reduced pressure. To the residue was added EtOAc (50 mL) and filtered through a pad of the Celite. The filter cake was washed with EtOAc (70 mL). The combined filtrate was concentrated under reduced pressure. The residue was purified by flash silica gel chromatography (Biotage; 20 SepaFlash® Silica Flash Column, Eluent of 0˜47% Petroleum ether/Ethyl acetate gradient @45 mL/min). The eluent was removed under reduced pressure. Compound tert-butyl N-[(3S)-1-(6-cyano-3-pyridyl)-3-piperidyl]carbamate (0.7 g, 2.32 mmol, 42.37% yield) was obtained as yellow solid. ¹H NMR (DMSO-d₆, 400 MHz) δ 8.37 (s, 1H), 7.71 (d, J=8.8 Hz, 1H), 7.30 (dd, J=2.8 Hz, 8.4 Hz, 1H), 7.01 (d, J=7.2 Hz, 1H), 3.81 (d, J=12.8 Hz, 2H), 3.38-3.36 (m, 1H), 3.01˜2.99 (m, 1H), 2.90 (dd, J=9.6 Hz, 12.8 Hz, 1H), 1.85˜1.81 (m, 1H), 1.75˜1.73 (m, 1H), 1.47 (t, J=8.8 Hz, 2H), 1.39 (s, 9H).

### Preparation of (S)-5-(3-aminopiperidin-1-yl)picolinonitrile (Step 2 in Scheme 3)

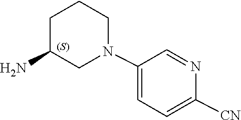

To a solution of tert-butyl N-[(3S)-1-(6-cyano-3-pyridyl)-3-piperidyl]carbamate (0.7 g, 2.32 mmol, 1 eq) in MeOH (2 mL) was added HCl/MeOH (4 M, 6 mL, 10.37 eq). The mixture was stirred at 20° C. for 3 hours. LCMS showed the reaction was complete. The reaction mixture was concentrated under reduced pressure. Compound 5-[(3S)-3-amino-1-piperidyl]pyridine-2-carbonitrile (0.7 g, crude, 3HCl) was obtained as yellow solid. ¹H NMR (DMSO-d₆, 400 MHz) δ 8.48-8.42 (m, 3H), 7.77 (d, J=8.8 Hz, 1H), 7.39 (dd, J=2.8 Hz, 8.8 Hz, 1H), 4.01˜3.98 (m, 1H), 3.74˜3.71 (m, 1H), 3.31˜3.26 (m, 1H), 3.21˜3.08 (m, 1H), 3.17˜3.12 (m, 1H), 2.02˜1.99 (m, 1H), 1.81˜1.78 (m, 1H), 1.70˜1.66 (m, 1H), 1.56˜1.55 (m, 1H).

### Preparation of 5-((3S)-3-(((9,10-difluoro-3-methyl-7-oxo-3,7-dihydro-2H-[1,4]oxazino[2,3,4-ij]quinolin-6-yl)methyl)amino)piperidin-1-yl)picolinonitrile (Step 3 in Scheme 3)

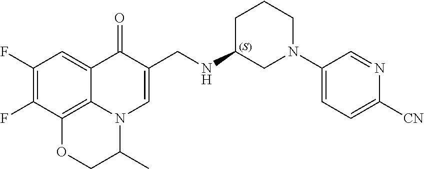

A solution of 5-[(3S)-3-amino-1-piperidyl]pyridine-2-carbonitrile (0.7 g, 2.25 mmol, 1 eq, 3HCl) and 6,7-difluoro-2-methyl-10-oxo-4-oxa-1-azatricyclo[7.3.1.0^{5,13}]trideca-5(13),6,8,11-tetra ene-11-carbaldehyde (595.72 mg, 2.25 mmol, 1 eq) in DCE (7 mL) was stirred at 20° C. for an hour. Then NaBH(OAc)₃ (952.12 mg, 4.49 mmol, 2 eq) was added in portions at 0° C. The resulting mixture was stirred at 20° C. for 11 hours. LCMS showed the reaction was nearly complete. The reaction mixture was quenched with ice water (30 mL) and then extracted with DCM (10 mL×3). The combined organic phase was washed with brine (10 mL×1), dried with anhydrous Na₂SO₄, filtered and concentrated in vacuum. Compound 5-[(3S)-3-[(6,7-difluoro-2-methyl-10-oxo-4-oxa-1-azatricyclo[7.3.1.0^{5,13}]trideca-5(13),6,8,11-tetraen-11-yl)methylamino]-1-piperidyl]pyridine-2-carbonitrile (0.92 g, crude) was obtained as yellow solid. ¹H NMR (DMSO-d₆, 400 MHz) δ 8.43 (s, 1H), 8.23˜8.22 (m, 1H), 7.73 (d, J=8.8 Hz, 1H), 7.62˜7.54 (m, 1H), 7.39-7.37 (m, 1H), 4.69-4.56 (m, 2H), 4.44˜4.41 (m, 1H), 4.05˜4.04 (m, 1H), 3.96 (d, J=4.8 Hz, 1H), 3.90 (s, 3H), 3.84˜3.76 (m, 1H), 3.07˜3.05 (m, 1H), 2.90˜2.85 (m, 1H), 2.00˜1.99 (m, 1H), 1.82˜1.74 (m, 1H), 1.52˜1.48 (m, 2H), 1.43˜1.38 (m, 3H).

### Compound 12

### Preparation of 5-((3S)-3-(((9,10-difluoro-3-methyl-7-oxo-3,7-dihydro-2H-[1,4]oxazino[2,3,4-ij]quinolin-6-yl)methyl)((2-methylpyridin-4-yl)methyl)amino)piperidin-1-yl)picolinonitrile (Step 4 in Scheme 3)

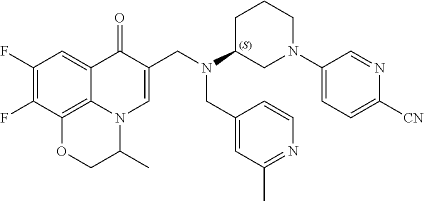

A solution of 5-[(3S)-3-[(6,7-difluoro-2-methyl-10-oxo-4-oxa-1-azatricyclo[7.3.1.0^{5,13}]trideca-5(13),6,8,11-tetraen-11-yl)methylamino]-1-piperidyl]pyridine-2-carbonitrile (0.15 g, 332.25 umol, 1 eq) and 2-methylpyridine-4-carbaldehyde (40.25 mg, 332.25 umol, 1 eq) in DCE (2 mL) was stirred at 20° C. for an hour. Then NaBH(OAc)₃ (140.83 mg, 664.50 umol, 2 eq) was added in portions at 0° C. The resulting mixture was stirred at 20° C. for 3 hours. LCMS and HPLC showed the reaction was complete. The mixture was quenched with ice-water (1 mL) and concentrated under reduced pressure. The residue was purified by prep-HPLC (column: Waters Xbridge BEH C18 100*30 mm*10 um; mobile phase: [water (NH₄HCO₃)-MeCN]; B %: 30%-60%, 10 mins). The eluent was removed under freeze drying. Compound 5-[(3S)-3-[(6,7-difluoro-2-methyl-10-oxo-4-oxa-1-azatricyclo[7.3.1.0^{5,13}]trideca-5(13),6,8,11-tetraen-11-yl) methyl-[(2-methyl-4-pyridyl)methyl]amino]-1-piperidyl]pyridine-2-carbonitrile (16.7 mg, 28.92 umol, 8.71% yield, 96.40% purity) was obtained as white solid. ¹H NMR (DMSO-d₆, 400 MHz) δ 8.41 (s, 1H), 8.28 (d, J=4.8 Hz, 1H), 8.09 (s, 1H), 7.68 (dd, J=6.8 Hz, 8.8 Hz, 1H), 7.55-7.54 (m, 1H), 7.35-7.34 (m, 1H), 7.24 (dd, J=5.2 Hz, 9.2 Hz, 2H), 4.69-4.67 (m, 1H), 4.57-4.55 (m, 1H), 4.40˜4.37 (m, 1H), 4.18 (d, J=11.6 Hz, 1H), 3.93 (d, J=12.8 Hz, 1H), 3.82˜3.75 (m, 1H), 3.72˜3.56 (m, 3H), 3.09 (t, J=12.0 Hz, 1H), 2.90˜2.83 (m, 1H), 2.63˜2.59 (m, 1H), 2.38 (s, 3H), 2.03˜2.00 (m, 1H), 1.78 (d, J=12.8 Hz, 1H), 1.69-1.64 (m, 1H), 1.42˜1.39 (m, 1H), 1.32 (dd, J=6.8 Hz, 11.2 Hz, 3H). MS: 96.40% (220 nm), 97.44% (254 nm). MS (ESI): mass calcd. For C₃₁H₃₀F₂N₆O₂ 556.24 m/z found 557.2 [M+H]⁺.

### Compound 13

### Preparation of 5-((3S)-3-(((9,10-difluoro-3-methyl-7-oxo-3,7-dihydro-2H-[1,4]oxazino[2,3,4-ij]quinolin-6-yl)methyl)((2-methylpyridin-4-yl)methyl)amino)piperidin-1-yl)picolinamide (Step 5 in Scheme 3)

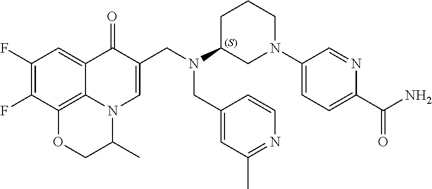

To a solution of 5-[(3S)-3-[(6,7-difluoro-2-methyl-10-oxo-4-oxa-1-azatricyclo[7.3.1.0^{5,13}]trideca-5(13),6,8,11-tetraen-11-yl)methyl-[(2-methyl-4-pyridyl)methyl]amino]-1-piperidyl]pyridine-2-carbonitrile (0.15 g, 269.49 umol, 1 eq) in DMSO (2 mL) and H₂O (0.2 mL) was added K₂CO₃ (74.49 mg, 538.98 umol, 2 eq) and UHP (0.13 g, 1.38 mmol, 5.13 eq) at 20° C. The reaction mixture was then stirred at 40° C. for 5 hours. LCMS and HPLC showed the reaction was complete. To the mixture was added water (30 mL) and then extracted with ethyl acetate (10 mL×3). The combined organic phase was washed with sat. Na₂SO₃ (10 mL×3), dried with anhydrous Na₂SO₄, filtered and concentrated in vacuum. The residue was purified by prep-HPLC (column: Waters Xbridge BEH C18 100*30 mm*10 um; mobile phase: [water (NH₄HCO₃)-MeCN]; B %: 30%-55%, 8 mins). The eluent was removed under freeze drying. Compound 5-[(3S)-3-[(6,7-difluoro-2-methyl-10-oxo-4-oxa-1-azatricyclo[7.3.1.0^{5,13}]trideca-5(13),6, 8,11-tetraen-11-yl)methyl-[(2-methyl-4-pyridyl)methyl]amino]-1-piperidyl]pyridine-2-carboxamide (48.1 mg, 83.71 umol, 31.06% yield, 100% purity) was obtained as white solid. ¹H NMR (DMSO-d₆, 400 MHz) δ 8.29-8.26 (m, 2H), 8.09 (s, 1H), 7.78 (dd, J=6.4 Hz, 8.8 Hz, 1H), 7.68˜7.63 (m, 1H), 7.58˜7.53 (m, 1H), 7.38˜7.34 (m, 1H), 7.28˜7.23 (m, 3H), 4.71˜4.68 (m, 1H), 4.60-4.55 (m, 1H), 4.40˜4.34 (m, 1H), 4.12 (d, J=12.0 Hz, 1H), 3.89-3.86 (m, 1H), 3.79-3.69 (m, 2H), 3.66-3.54 (m, 2H), 3.04 (t, J=11.6 Hz, 1H), 2.83˜2.76 (m, 1H), 2.70-2.64 (m, 1H), 2.38 (s, 3H), 2.04˜2.01 (m, 1H), 1.79 (d, J=12.8 Hz, 1H), 1.67˜1.60 (m, 1H), 1.49-1.43 (m, 1H), 1.32 (dd, J=6.8 Hz, 8.4 Hz, 3H). MS: 100.00% (220 nm), 100.00% (254 nm). MS (ESI): mass calcd. For C₃₁H₃₂F₂N₆O₃ 574.25 m/z found 575.3 [M+H]⁺.

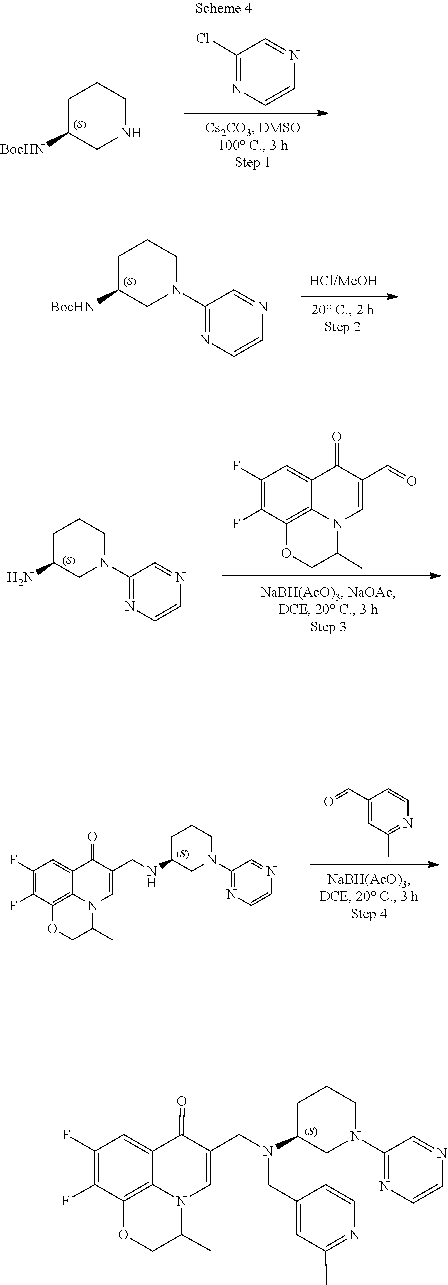

### Specific Procedures for Preparing Compounds in Scheme 4

### Preparation of (S)-tert-butyl (1-(pyrazin-2-yl)piperidin-3-yl)carbamate (Step 1 in Scheme 4)

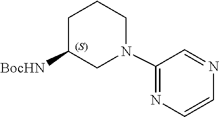

To a mixture of tert-butyl N-[(3S)-3-piperidyl]carbamate (2 g, 9.99 mmol, 1 eq) and 2-chloropyrazine (1.26 g, 10.98 mmol, 982.90 uL, 1.1 eq) in DMSO (20 mL) was added Cs₂CO₃ (6.51 g, 19.97 mmol, 2 eq). The reaction mixture was stirred at 100° C. for 3 hours. LCMS indicated tert-butyl N-[(3S)-3-piperidyl]carbamate was consumed and one major peak with desired mass was detected. The reaction mixture was cooled to 0° C., quenched by addition H₂O (50 mL) at 0° C. and then extracted with EtOAc (40 mL×2). The combined organic layers were washed with brine (40 mL×3), dried over anhydrous Na₂SO₄, filtered and concentrated under reduced pressure. The residue was purified by flash silica gel chromatography (Biotage®; 25 g SepaFlash® Silica Flash Column, Eluent of 0˜50% ethyl acetate/petroleum ether gradient @75 mL/min). The eluent was concentrated in vacuum. Compound tert-butylN-(1-pyrazin-2-yl-3-piperidyl)carbamate (1.4 g, 5.03 mmol, 50.37% yield) was obtained as yellow solid. ¹H NMR (DMSO-d₆, 400 MHz) δ 8.27 (s, 1H), 8.04 (s, 1H), 7.78 (s, 1H), 6.95 (d, J=7.6 Hz, 1H), 4.18 (d, J=11.2 Hz, 1H), 4.04 (d, J=12.4 Hz, 1H), 3.28-3.27 (m, 1H), 2.96 (t, J=10.4 Hz, 1H), 2.80 (t, J=12.4 Hz, 1H), 1.84 (s, 1H), 1.74 (s, 1H), 1.46˜1.42 (m, 2H), 1.39 (s, 9H).

### Preparation of (S)-1-(pyrazin-2-yl)piperidin-3-amine (Step 2 in Scheme 4)

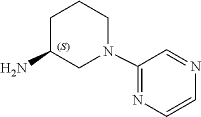

To a solution of tert-butyl N-(1-pyrazin-2-yl-3-piperidyl)carbamate (1.4 g, 5.03 mmol, 1 eq) in MeOH (2 mL) was added HCl/MeOH (4 M, 40 mL). The reaction mixture was stirred at 20° C. for 2 hours. LCMS indicated tert-butyl N-(1-pyrazin-2-yl-3-piperidyl)carbamate was nearly consumed and one major peak with desired mass was detected. The mixture was concentrated in vacuum. Compound (3S)-1-pyrazin-2-ylpiperidin-3-amine (1.2 g, 4.17 mmol, 82.95% yield, 3HCl) was obtained as yellow solid. ¹H NMR (DMSO-d₆, 400 MHz) δ 8.37 (s 1H), 8.28 (s, 2H), 8.13 (s, 1H), 7.86 (s, 1H), 4.30 (d, J=10.0 Hz, 1H), 3.96˜3.90 (m, 1H), 3.26˜3.14 (m, 3H), 2.04˜2.00 (m, 1H), 1.81˜1.77 (m, 1H), 1.70˜1.61 (m, 1H), 1.58˜1.48 (m, 1H).

### Preparation of 9,10-difluoro-3-methyl-6-((((S)-1-(pyrazin-2-yl)piperidin-3-yl)amino)methyl)-2H-[1,4]oxazino[2,3,4-ij]quinolin-7(3H)-one (Step 3 in Scheme 4)

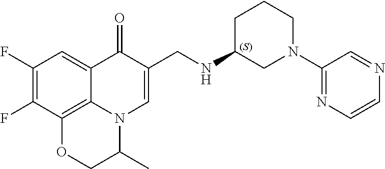

To a mixture of 6,7-difluoro-2-methyl-10-oxo-4-oxa-1-azatricyclo[7.3.1.0^{5,13}]trideca-5(13), 6,8,11-tetraene-11-carbaldehyde (0.3 g, 1.13 mmol, 1 eq) and (3S)-1-pyrazin-2-ylpiperidin-3-amine (325.34 mg, 1.13 mmol, 1 eq, 3HCl) in DCE (8 mL) was added NaOAc (102.07 mg, 1.24 mmol, 1.1 eq) and NaBH(OAc)₃ (359.61 mg, 1.70 mmol, 1.5 eq). The reaction mixture was stirred at 20° C. for 3 hours. LCMS indicated the reaction was complete. The reaction mixture was quenched by addition H₂O (15 mL) at 0° C. and then extracted with EtOAc (20 mL×3). The combined organic phase was washed with brine (10 mL×1), dried over anhydrous Na₂SO₄, filtered and concentrated in vacuum. Compound 6,7-difluoro-2-methyl-11-[[[(3S)-1-pyrazin-2-yl-3-piperidyl]amino]methyl]-4-oxa-1-azatricyclo[7.3.1.0^{5,13}]trideca-5(13),6,8,11-tetraen-10-one (310 mg, crude) was obtained as white solid. ¹H NMR (DMSO-d₆, 400 MHz) δ 8.30 (s, 1H), 8.08 (d, J=5.2 Hz, 1H), 8.04-8.01 (m, 1H), 7.73 (s, 1H), 7.56 (dd, J=8.0 Hz, 10.4 Hz, 1H), 4.62˜4.57 (m, 2H), 4.33˜4.24 (m, 4H), 4.04˜4.01 (m, 2H), 3.94 (s, 1H), 3.05-2.94 (m, 1H), 2.86˜2.77 (m, 1H), 1.95˜1.86 (m, 4H), 1.38˜1.36 (m, 3H).

### Compound 14

### Preparation of 9,10-difluoro-3-methyl-6-((((2-methylpyridin-4-yl)methyl)((S)-1-(pyrazin-2-yl)piperidin-3-yl)amino)methyl)-2H-[1,4]oxazino[2,3,4-ij]quinolin-7(3H)-one (Step 4 in Scheme 4)

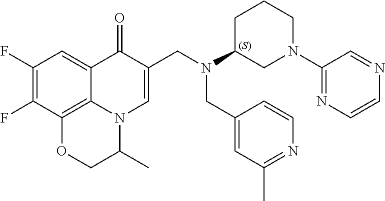

To a mixture of 6,7-difluoro-2-methyl-11-[[[(3S)-1-pyrazin-2-yl-3-piperidyl]amino]methyl]-4-oxa-1-azatricyclo[7.3.1.0^{5,13}]trideca-5(13),6,8,11-tetraen-10-one (300 mg, 701.84 umol, 1 eq) and 2-methylpyridine-4-carbaldehyde (127.53 mg, 1.05 mmol, 1.5 eq) in DCE (6 mL) was added NaBH(OAc)₃ (297.50 mg, 1.40 mmol, 2 eq). The mixture was stirred at 20° C. for 3 hours. LCMS indicated the reaction was complete. The reaction mixture was quenched by addition H₂O (15 mL) at 0° C. and then extracted with DCM (20 mL×3). The combined organic layers were washed with brine (15 mL×2), dried over anhydrous Na₂SO₄, filtered and concentrated under reduced pressure. The residue was purified by prep-HPLC (column: Waters Xbridge BEH C18 100*30 mm*10 um; mobile phase: [water (NH₄HCO₃-MeCN]; B %: 25%-55%, 10 mins). The eluent was dried over lyophilization. Compound 6,7-difluoro-2-methyl-11-[[(2-methyl-4-pyridyl)methyl-[(3S)-1-pyrazin-2-yl-3-piperidyl]amino]methyl]-4-oxa-1-azatricyclo[7.3.1.0^{5,13}]trideca-5(13),6,8,11-tetraen-10-one (136.8 mg, 256.86 umol, 36.60% yield, 100% purity) was obtained as white solid. ¹H NMR (DMSO-d₆, 400 MHz) δ 8.32 (s, 1H), 8.27 (d, J=4.8 Hz, 1H), 8.07 (s, 1H), 8.03˜8.00 (m, 1H), 7.74 (t, J=2.4 Hz, 1H), 7.54 (dd, J=8.4 Hz, 10.0 Hz, 1H), 7.25 (s, 1H), 7.21 (t, J=4.4 Hz, 1H), 4.68˜4.65 (m, 1H), 4.57-4.51 (m, 2H), 4.39˜4.35 (m, 1H), 4.26 (d, J=12.8 Hz, 1H), 3.84-3.66 (m, 3H), 3.60-3.56 (m, 1H), 2.98 (t, J=12.0 Hz, 1H), 2.79 (t, J=12.8 Hz, 1H), 2.63˜2.57 (m, 1H), 2.36 (d, J=3.6 Hz, 3H), 2.02 (d, J=11.2 Hz, 1H), 1.77 (d, J=13.2 Hz, 1H), 1.70˜1.61 (m, 1H), 1.42˜1.38 (m, 1H), 1.32 (dd, J=6.8 Hz, 14.8 Hz, 3H). LCMS: 100% (220 nm), 100% (254 nm). MS (ESI): mass calcd. For C₂₉H₃₀F₂N₆O₂ 532.24 m/z found 533.3 [M+H]⁺.

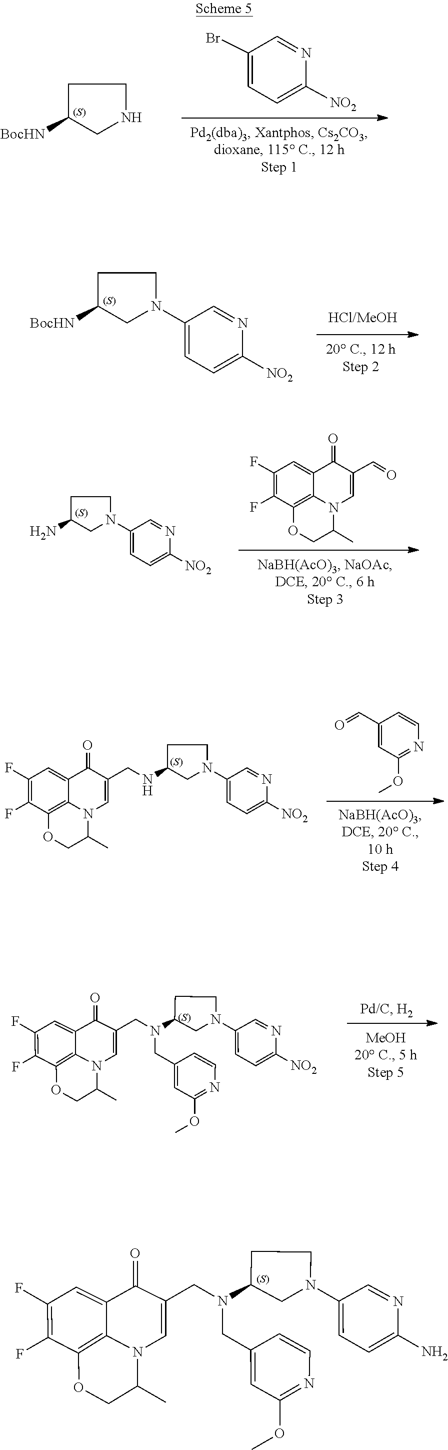

### Specific Procedures for Preparing Compounds in Scheme 5

### Preparation of (S)-tert-butyl (1-(6-nitropyridin-3-yl)pyrrolidin-3-yl)carbamate (Step 1 in Scheme 5)

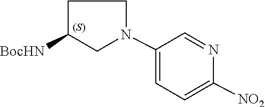

A mixture of tert-butyl N-pyrrolidin-3-ylcarbamate (1.79 g, 9.61 mmol, 1.3 eq), 5-bromo-2-nitro-pyridine (1.5 g, 7.39 mmol, 1 eq), Pd₂(dba)₃ (338.33 mg, 369.47 umol, 0.05 eq), Xantphos (213.78 mg, 369.47 umol, 0.05 eq) and Cs₂CO₃ (3.25 g, 9.98 mmol, 1.35 eq) in dioxane (30 mL) was degassed and purged with N₂ for 3 times. The reaction mixture was then stirred at 115° C. for 12 hours under N₂ atmosphere. LCMS showed the reaction was complete. The mixture was cooled to room temperature and filtered through a pad of the Celite. The filter cake was washed with EtOAc (100 mL). The combined filtrate was concentrated under reduced pressure. The residue was purified by flash silica gel chromatography (Biotage; 40 SepaFlash® Silica Flash Column, Eluent of 0-45% Ethyl acetate/Petroleum ether gradient @60 mL/min). The solvent was removed under reduced pressure. Compound tert-butyl N-[1-(6-nitro-3-pyridyl)pyrrolidin-3-yl]carbamate (1.8 g, 5.84 mmol, 79.00% yield) was obtained as yellow solid. ¹H NMR (400 MHz, DMSO-d₆) δ 8.15 (d, J=8.8 Hz, 1H), 7.87 (s, 1H), 7.28 (d, J=6.8 Hz, 1H), 7.05 (dd, J=2.8 Hz, 9.2 Hz, 1H), 4.21˜4.18 (m, 1H), 3.64 (dd, J=6.4 Hz, 10.8 Hz, 1H), 3.56˜3.51 (m, 1H), 3.48˜3.42 (m, 1H), 3.25 (dd, J=4.4 Hz, 10.8 Hz, 1H), 2.21˜2.13 (m, 1H), 1.97-1.91 (m, 1H), 1.39 (s, 9H).

### Preparation of (S)-1-(6-nitropyridin-3-yl)pyrrolidin-3-amine (Step 2 in Scheme 5)

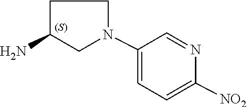

A solution of tert-butyl N-[1-(6-nitro-3-pyridyl)pyrrolidin-3-yl]carbamate (1.8 g, 5.84 mmol, 1 eq) in HCl/MeOH (4 M, 30 mL, 34.97 eq) was stirred at 20° C. for 12 hours. LC-MS showed the reaction was complete. The reaction mixture was concentrated under reduced pressure. Compound (3S)-1-(6-nitro-3-pyridyl)pyrrolidin-3-amine (1.8 g, crude, 3HCl) was obtained as brown solid. ¹H NMR (400 MHz, DMSO-d₆) δ 8.45 (s, 2H), 8.20 (d, J=9.2 Hz, 1H), 7.92 (s, 1H), 7.13 (dd, J=2.8 Hz, 9.2 Hz, 1H), 4.05-3.98 (m, 1H), 3.74 (dd, J=1.2 Hz, 6.4 Hz, 1H), 3.69-3.63 (m, 1H), 3.57-3.53 (m, 2H), 2.40-2.31 (m, 1H), 2.22˜2.17 (m, 1H).

### Preparation of 9,10-difluoro-3-methyl-6-((((S)-1-(6-nitropyridin-3-yl)pyrrolidin-3-yl)amino)methyl)-2H-[1,4]oxazino[2,3,4-ij]quinolin-7(3H)-one (Step 3 in Scheme 5)

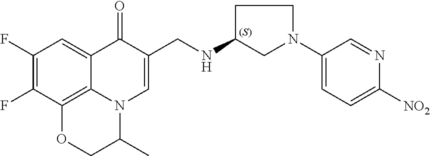

To a mixture of 6,7-difluoro-2-methyl-10-oxo-4-oxa-1-azatricyclo[7.3.1.05,13]trideca-5(13),6, 8,11-tetraene-11-carbaldehyde (835.05 mg, 3.15 mmol, 1 eq) and (3S)-1-(6-nitro-3-pyridyl) pyrrolidin-3-amine (1 g, 3.15 mmol, 1 eq, 3HCl) in DCE (20 mL) was added NaOAc (284.11 mg, 3.46 mmol, 1.1 eq) at 20° C. under N₂. The reaction mixture was stirred at 20° C. for half an hour and then NaBH(OAc)₃ (1.00 g, 4.72 mmol, 1.5 eq) was added at 20° C. The reaction mixture was stirred at 20° C. for 6 hours. LC-MS showed the reaction was complete. The reaction mixture was quenched by addition water (50 mL) at 0° C. and extracted with EtOAc (50 mL×3). The combined organic layers were washed with brine (50 mL×2), dried over Na₂SO₄, filtered and concentrated under reduced pressure. Compound 6,7-difluoro-2-methyl-11-[[[(3S)-1-(6-nitro-3-pyridyl) pyrrolidin-3-yl]amino]methyl]-4-oxa-1-azatricyclo[7.3.1.05,13]trideca-5(13),6,8,11-tetraen-10-one (1 g, crude) was obtained as brown solid. ¹H NMR (400 MHz, DMSO-d₆) δ 8.15 (d, J=9.2 Hz, 1H), 8.06 (s, 1H), 7.86 (d, J=2.4 Hz, 1H), 7.57 (dd, J=8.0 Hz, 10.8 Hz, 1H), 7.03 (dd, J=2.8 Hz, 8.8 Hz, 1H), 4.56 (d, J=11.2 Hz, 1H), 4.41˜4.36 (m, 1H), 3.64˜3.55 (m, 4H), 3.47˜3.45 (m, 2H), 3.32˜3.28 (m, 2H), 3.17 (d, J=3.6 Hz, 1H), 2.17-2.11 (m, 1H), 1.97-1.90 (m 1H), 1.39 (dd, J=6.8 Hz, 8.8 Hz, 3H).

### Preparation of 9,10-difluoro-6-((((2-methoxypyridin-4-yl)methyl)((S)-1-(6-nitropyridin-3-yl)pyrrolidin-3-yl)amino)methyl)-3-methyl-2H-[1,4]oxazino[2,3,4-ij]quinolin-7(3H)-one (Step 4 in Scheme 5)

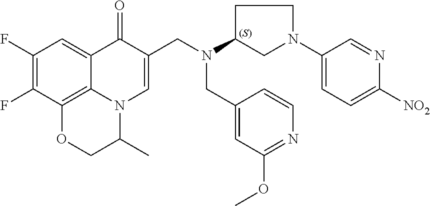

To a mixture of 6,7-difluoro-2-methyl-11-[[[(3S)-1-(6-nitro-3-pyridyl)pyrrolidin-3-yl]amino]methyl]-4-oxa-1-azatricyclo[7.3.1.05,13]trideca-5(13),6,8,11-tetraen-10-one (200 mg, 437.23 umol, 1 eq) and 2-methoxypyridine-4-carbaldehyde (59.96 mg, 437.23 umol, 1 eq) in DCE (3 mL) was added NaBH(OAc)₃ (139.00 mg, 655.84 umol, 1.5 eq) at 20° C. under N₂. Then the mixture was stirred at 20° C. for 10 hours. LC-MS showed 6,7-difluoro-2-methyl-11-[[[(3S)-1-(6-nitro-3-pyridyl)pyrrolidin-3-yl]amino]methyl]-4-oxa-1-azatricyclo[7.3.1.05,13]trideca-5(13),6,8,11-tetraen-10-one was consumed completely and one new peak with desired m/z was detected. The reaction mixture was quenched by addition water (50 mL) at 25° C., and extracted with EtOAc (50 mL×3). The combined organic layers were washed with brine (50 mL×2), dried over Na₂SO₄, filtered and concentrated under reduced pressure to give a residue. Compound 6,7-difluoro-11-[[(2-methoxy-4-pyridyl)methyl-[(3S)-1-(6-nitro-3-pyridyl)pyrrolidin-3-yl]amino]methyl]-2-methyl-4-oxa-1-azatricyclo[7.3.1.05,13]trideca-5(13),6,8,11-tetraen-10-one (200 mg, crude) was obtained as brown solid. ¹H NMR (400 MHz, DMSO-d₆) δ 8.14-8.11 (m, 1H), 8.08-8.04 (m, 1H), 7.97-7.95 (m, 1H), 7.83 (d, J=2.8 Hz, 1H), 7.56-7.51 (m, 1H), 7.01˜6.98 (m, 1H), 6.96-6.93 (m, 1H), 6.79-6.76 (m, 1H), 4.64-4.59 (m, 1H), 4.55-4.52 (m, 1H), 4.49˜4.48 (m, 1H), 4.39˜4.31 (m, 1H), 3.93˜3.92 (m, 1H), 3.72 (s, 3H), 3.68˜3.66 (m, 2H), 3.62˜3.59 (m, 3H), 3.47˜3.45 (m, 1H), 3.39˜3.37 (m, 1H), 1.33˜1.32 (m, 2H), 1.19˜1.16 (m, 3H).

### Compound 15

### Preparation of 6-((((S)-1-(6-aminopyridin-3-yl)pyrrolidin-3-yl)((2-methoxypyridin-4-yl)methyl)amino)methyl)-9,10-difluoro-3-methyl-2H-[1,4]oxazino[2,3,4-ij]quinolin-7(3H)-one (Step 5 in Scheme 5)

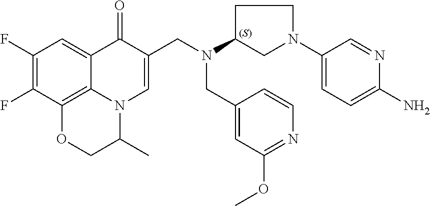

A mixture of 6,7-difluoro-11-[[(2-methoxy-4-pyridyl)methyl-[(3S)-1-(6-nitro-3-pyridyl) pyrrolidin-3-yl]amino]methyl]-2-methyl-4-oxa-1-azatricyclo[7.3.1.05,13]trideca-5(13),6,8,11-tetraen-10-one (200 mg, 345.68 umol, 1 eq) and Pd/C (60 mg, 10% purity, 1 eq) in MeOH (50 mL) was stirred at 25° C. for 5 hours under H₂ (15 psi). LC-MS and HPLC showed 6,7-difluoro-11-[[(2-methoxy-4-pyridyl)methyl-[(3S)-1-(6-nitro-3-pyridyl)pyrrolidin-3-yl]amino]methyl]-2-methyl-4-oxa-1-azatricyclo[7.3.1.05, 13]trideca-5(13),6,8,11-tetraen-10-one was consumed completely and one major peak with desired mass was detected. The reaction mixture was filtered through a pad of the Celie. The filtrate was concentrated under reduced pressure. The residue was purified by prep-HPLC (column: Waters Xbridge Prep OBD C 18 150*40 mm*10 um; mobile phase: [water (NH₄HCO₃)-MeCN]; B %: 25%-55%, 8 mins). The solvent was removed under freeze drying. Compound 11-[[[(3S)-1-(6-amino-3-pyridyl)pyrrolidin-3-yl]-[(2-methoxy-4-pyridyl)methyl]amino]methyl]-6,7-difluoro-2-methyl-4-oxa-1-azatricyclo[7.3.1.05,13]trideca-5(13),6,8,11-tetraen-10-one (3.1 mg, 5.65 umol, 1.63% yield) was obtained pale yellow solid. ¹H NMR (400 MHz, DMSO-d₆) δ 13.96-13.90 (m, 1H), 8.46 (s, 1H), 8.06 (s, 1H), 7.71˜7.70 (m, 1H), 7.57-7.56 (m, 1H), 7.43˜7.41 (m, 2H), 7.27 (s, 1H), 7.22˜7.21 (m, 1H), 7.01˜6.99 (m, 1H), 4.56-4.54 (m, 2H), 4.39 (s, 3H), 4.32˜4.18 (m, 3H), 4.00˜3.93 (m, 2H), 3.78˜3.75 (m, 3H), 3.56˜3.54 (m, 1H), 3.50˜3.46 (m, 1H), 3.13˜3.12 (m, 1H), 2.67-2.63 (m, 1H), 1.47-1.43 (m, 3H). HPLC: 98.76% (220 nm), 98.51% (254 nm). MS (ESI): mass calcd. For C₂₉H₃₀F₂N₆O₃ 548.23 m/z found 549.3 [M+H]⁺.

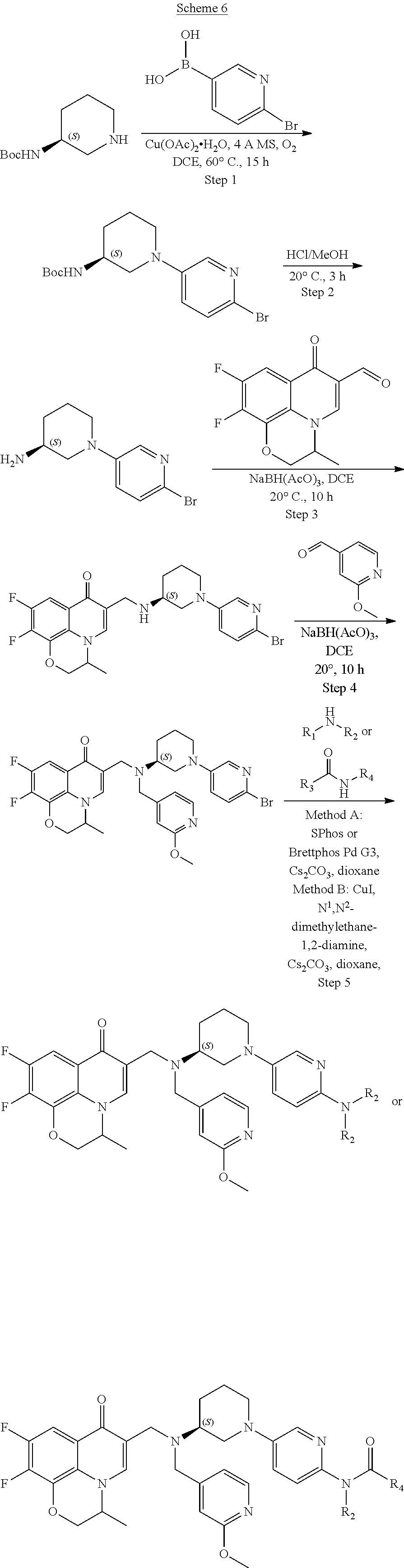

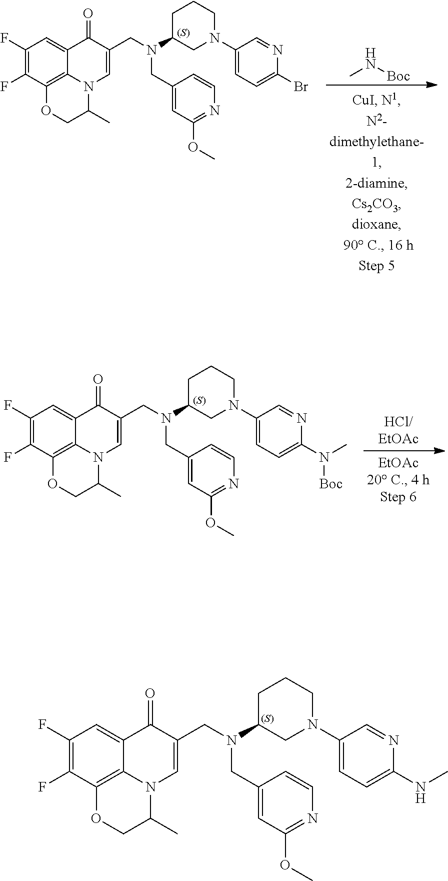

### General Procedures for Preparing Compounds in Scheme 6

### Preparation of (S)-tert-butyl (1-(6-bromopyridin-3-yl)piperidin-3-yl)carbamate (Step 1 in Scheme 6

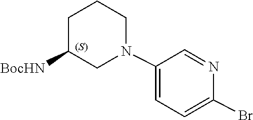

A mixture of (6-bromo-3-pyridyl)boronic acid (10.08 g, 49.93 mmol, 2 eq), 4A MS (10 g, 1.00 eq) and Cu(OAc)₂·H₂O (498.43 mg, 2.50 mmol, 498.43 uL, 0.1 eq) in DCE (100 mL) was stirred at 20° C. for 5 minutes and then tert-butyl N-[(3S)-3-piperidyl]carbamate (5 g, 24.97 mmol, 1 eq) was added. The reaction mixture was stirred at 60° C. for 15 hours under O₂ (1 atm). LCMS and TLC (Petroleum ether:Ethyl acetate=2:1, R_{f}=0.4) indicated (6-bromo-3-pyridyl)boronic acid was remained and one peak with desired mass was formed. The reaction mixture was filtered through a pad of the Celite. The filtrate was concentrated under reduced pressure. The residue was purified by flash silica gel chromatography (Biotage®; 80 g SepaFlash® Silica Flash Column, Eluent of 0˜45% Ethyl acetate/Petroleum ether gradient @110 mL/min). The eluent was concentrated in vacuum. Compound tert-butyl N-[(3S)-1-(6-bromo-3-pyridyl)-3-piperidyl]carbamate (3.2 g, 8.98 mmol, 35.98% yield) was obtained as yellow solid. ¹H NMR (DMSO-d₆, 400 MHz) δ 8.14 (s, 1H), 7.37 (d, J=8.8 Hz, 1H), 7.28 (d, J=5.6 Hz, 1H), 6.94 (d, J=7.6 Hz, 1H), 3.64-3.57 (m, 2H), 3.42˜3.33 (m, 1H), 2.75 (t, J=10.8 Hz, 1H), 2.62 (t, J=10.0 Hz, 1H), 1.99˜1.87 (m, 1H), 1.70˜1.63 (m, 1H), 1.54˜1.50 (m, 2H), 1.39 (s, 9H).

### Preparation of (S)-1-(6-bromopyridin-3-yl)piperidin-3-amine (Step 2 in Scheme 6)

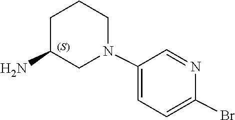

A mixture of tert-butyl N-[(3S)-1-(6-bromo-3-pyridyl)-3-piperidyl]carbamate (1 g, 2.81 mmol, 1 eq) in HCl/MeOH (4 M, 14.03 mL, 20 eq) was stirred at 20° C. for 3 hours. LCMS indicated the reaction was complete. The reaction mixture was concentrated under reduced pressure. Compound (3S)-1-(6-bromo-3-pyridyl)piperidin-3-amine (0.7 g, 1.92 mmol, 68.23% yield, 3HCl) was obtained as yellow solid. ¹H NMR (DMSO-d₆, 400 MHz) δ 8.08 (s, 1H), 8.04 (s, 3H), 7.45 (d, J=8.4 Hz, 1H), 7.35-7.32 (m, 1H), 3.44-3.40 (m, 2H), 3.27-3.23 (m, 1H), 3.17˜2.98 (m, 2H), 1.94˜1.91 (m, 1H), 1.80˜1.76 (m, 1H), 1.61˜1.52 (m, 2H).

### Preparation of 6-((((S)-1-(6-bromopyridin-3-yl)piperidin-3-yl)amino)methyl)-9,10-difluoro-3-methyl-2H-[1,4]oxazino[2,3,4-ij]quinolin-7(3H)-one (Step 3 in Scheme 6)

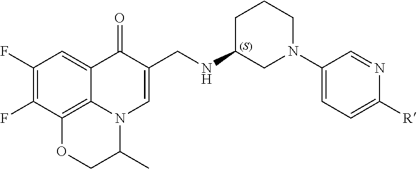

A solution of 9,10-difluoro-3-methyl-7-oxo-3,7-dihydro-2H-[1,4]oxazino[2,3,4-ij]quinoline-6-carbaldehyde (0.7 g, 2.64 mmol, 1 eq) and (3S)-1-(6-bromo-3-pyridyl)piperidin-3-amine (964.77 mg, 2.64 mmol, 1 eq, 3HCl) in DCE (20 mL) was stirred at 20° C. for half an hour. Then the mixture was cooled to 0° C., NaBH(OAc)₃ (1.12 g, 5.28 mmol, 2 eq) was added in portions at 0° C. The mixture was stirred at 20° C. for 10 hours. LCMS and TLC (EtOAc:MeOH=6:1, R_{f}=0.3) showed the reaction was complete. The reaction mixture was quenched with ice water (20 mL) at 0° C. and made pH=8 with sat. NaHCO₃. The mixture was extracted with DCM and i-PrOH (v:v=3:1, 30 mL×3). The combined organic layer was washed with brine (20 mL×1), dried over Na₂SO₄, filtered and concentrated under reduced pressure. Compound 11-[[[(3S)-1-(6-bromo-3-pyridyl)-3-piperidyl]amino]methyl]-6,7-difluoro-2-methyl-4-oxa-1-azatricyclo[7.3.1.0^{5,13}]trideca-5(13),6,8, 11-tetraen-10-one (1 g, 1.98 mmol, 74.97% yield) was obtained as yellow solid. ¹H NMR (DMSO-d₆, 400 MHz) δ 8.11-8.09 (m, 1H), 8.06-8.04 (m, 1H), 7.59-7.55 (m, 1H), 7.33˜7.31 (m, 2H), 4.64-4.55 (m, 3H), 4.42˜4.39 (m, 2H), 3.79-3.75 (m, 1H), 3.53˜3.50 (m, 1H), 2.80-2.74 (m, 1H), 2.67-2.64 (m, 2H), 1.90-1.88 (m, 1H), 1.77˜1.70 (m, 1H), 1.54˜1.45 (m, 1H), 1.40˜1.36 (m, 3H), 1.34˜1.25 (m, 1H).

### Preparation of 6-((((S)-1-(6-bromopyridin-3-yl)piperidin-3-yl)((2-methoxypyridin-4-yl)methyl)amino)methyl)-9,10-difluoro-3-methyl-2H-[1,4]oxazino[2,3,4-ij]quinolin-7(3H)-one (Step 4 in Scheme 6)

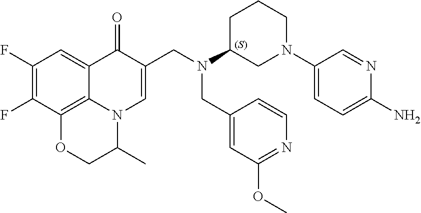

A solution of 11-[[[(3S)-1-(6-bromo-3-pyridyl)-3-piperidyl]amino]methyl]-6,7-difluoro-2-methyl-4-oxa-1-azatricyclo[7.3.1.0^{5,13}]trideca-5(13),6,8,11-tetraen-10-one (1 g, 1.68 mmol, 85% purity, 1 eq) and 2-methoxypyridine-4-carbaldehyde (345.99 mg, 2.52 mmol, 1.5 eq) in DCE (20 mL) was stirred at 25° C. for half an hour. Then the mixture was cooled to 0° C., NaBH(OAc)₃ (712.96 mg, 3.36 mmol, 2 eq) was added in portions at 0° C. The mixture was stirred at 20° C. for 10 hours. LCMS and TLC (EtOAc:MeOH=8:1, R_{f}=0.3) showed the reaction was nearly complete. The reaction mixture was quenched with ice water (20 mL) at 0° C. and made pH=8 with sat. NaHCO₃. The mixture was extracted with DCM and i-PrOH (v:v=3:1, 30 mL×3). The combined organic layer was washed with brine (20 mL×1), dried over Na₂SO₄, filtered and concentrated under reduced pressure. The residue was purified by flash silica gel chromatography (Biotage®; 20 g SepaFlash® Silica Flash Column, Eluent of 0˜10% Methanol/Ethyl acetate gradient @80 mL/min). The eluent was concentrated in vacuum. Compound 11-[[[(3S)-1-(6-bromo-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (0.7 g, 1.12 mmol, 66.43% yield) was obtained as pale yellow solid. ¹H NMR (DMSO-d₆, 400 MHz) δ 8.09 (s, 1H), 8.05 (s, 1H), 8.03˜8.00 (m, 1H), 7.55-7.53 (m, 1H), 7.32˜7.29 (m, 2H), 7.02˜7.00 (m, 1H), 6.85-6.83 (m, 1H), 4.69˜4.67 (m, 1H), 4.56-4.55 (m, 1H), 4.40˜4.38 (m, 1H), 4.01˜3.82 (m, 2H), 3.76 (s, 3H), 3.73˜3.55 (m, 4H), 2.85˜2.81 (m, 1H), 2.70-2.65 (m, 2H), 1.98-1.96 (m, 1H), 1.77-1.74 (m, 1H), 1.54-1.42 (m, 1H), 1.40-1.36 (m, 1H), 1.34˜1.30 (m, 3H).

### Preparation of Compounds in Scheme 6 (Step 5 in Scheme 6)

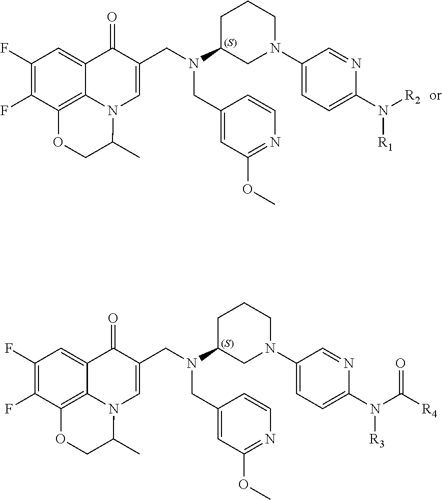

Method A: A mixture of 11-[[[(3S)-1-(6-bromo-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (111.73 umol, 1 eq), different amine (335.20 umol˜558.65 umol, 3 eq˜5 eq), Cs₂CO₃ (223.47 umol-333.71 umol, 2 eq˜3 eq) and SPhos or BrettPhos Pd G3 (16.76 umol, 0.15 eq) in dioxane (10 mL/mmol˜18 mL/mmol) was stirred at 80° C.˜100° C. for 10 hours˜16 hours under N₂. The mixture was filtered through a pad of the Celite and then the filtrate was concentrated under reduced pressure. The residue was purified by prep-HPLC (column: Waters Xbridge Prep OBD C 18 150*40 mm*10 um or Waters Xbridge BEH C18 100*30 mm*10 um or Phenomenex C18 75*30 mm*3 um; mobile phase: [water (NH₄HCO₃)-MeCN]; B %: 25%-70%, 8 mins or 10 mins) or by flash silica gel chromatography (Biotage®; 12 g SepaFlash® Silica Flash Column, Eluent of 0˜100% Ethyl acetate/Petroleum ether gradient @45 mL/min). The eluent was removed under freeze drying to give the desired product.
Method B: A mixture of 11-[[[(3S)-1-(6-bromo-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (159.62 umol, 1 eq), different amide (478.86 umol, ˜798.10 umol, 3 eq˜5 eq), CuI (79.81 umol, 0.5 eq), N1,N2-dimethylethane-1,2-diamine (31.92 umol, 0.2 eq) and Cs₂CO₃ (319.24 umol-478.86 umol, 2 eq˜3 eq) in dioxane (10 mL/mmol˜30 mL/mmol) was stirred at 80° C.˜100° C. for 10 hours˜16 hours under N₂. The mixture was filtered through a pad of the Celite and then the filtrate was concentrated under reduced pressure. The residue was purified by prep-HPLC (column: Waters Xbridge Prep OBD C 18 150*40 mm*10 um or Waters Xbridge BEH C18 100*30 mm*10 um or Phenomenex C18 75*30 mm*3 um; mobile phase: [water (NH₄HCO₃)-MeCN]; B %: 25%-70%, 8 mins or 10 mins) or by flash silica gel chromatography (Biotage®; 12 g SepaFlash® Silica Flash Column, Eluent of 0-100% Ethyl acetate/Petroleum ether gradient @45 mL/min). The eluent was removed under freeze drying to give the desired product.

### Compound 16

### Preparation of tert-butyl (5-((3S)-3-(((9,10-difluoro-3-methyl-7-oxo-3,7-dihydro-2H-[1,4]oxazino[2,3,4-ij]quinolin-6-yl)methyl)((2-methoxypyridin-4-yl)methyl)amino)piperidin-1-yl)pyridin-2-yl)(methyl)carbamate (Step 5 in Scheme 6)

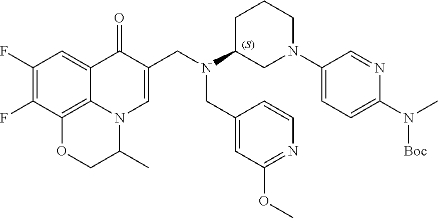

A mixture of 11-[[[(3S)-1-(6-bromo-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (100 mg, 159.62 umol, 1 eq), tert-butyl N-methylcarbamate (83.75 mg, 638.48 umol, 4 eq), Cs₂CO₃ (104.01 mg, 319.24 umol, 2 eq), CuI (15.20 mg, 79.81 umol, 0.5 eq) and N,N′-dimethylethane-1,2-diamine (7.04 mg, 79.81 umol, 8.59 uL, 0.5 eq) in dioxane (3 mL) was degassed and purged with N₂ for 3 times, and then the mixture was stirred at 90° C. for 16 hours under N₂ atmosphere. LCMS and HPLC indicated 11-[[[(3S)-1-(6-bromo-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one was consumed and one new peak with desired MS was detected. The reaction mixture was cooled to room temperature, filtered through celite and washed with EtOAc (10 mL). The filtrate was concentrated under reduced pressure. The residue was purified by flash silica gel chromatography (Biotage®; 12 g SepaFlash® Silica Flash Column, Eluent of 0-100% Ethyl acetate/Petroleum ether gradient @45 mL/min). The eluent was concentrated in vacuum. Compound tert-butyl N-[5-[(3S)-3-[(6,7-difluoro-2-methyl-10-oxo-4-oxa-1-azatricyclo[7.3.1.0^{5,13}]trideca-5(13),6,8,11-tetraen-11-yl)methyl-[(2-methoxy-4-pyridyl)methyl]amino]-1-piperidyl]-2-pyridyl]-N-methyl-carbamate (70 mg, 103.44 umol, 32.40% yield) was obtained as yellow solid. Two batches were carried out and workup together. ¹H NMR (DMSO-d₆, 400 MHz) δ 8.11 (d, J=5.2 Hz, 1H), 8.00 (d, J=6.0 Hz, 1H), 7.54 (t, J=8.0 Hz, 1H), 7.46-7.38 (m, 1H), 7.35-7.31 (m, 1H), 7.02 (t, J=4.0 Hz, 1H), 6.85 (d, J=5.6 Hz, 1H), 6.79-6.73 (m, 1H), 4.70 (d, J=6.8 Hz, 1H), 4.60-4.58 (m, 1H), 4.39 (d, J=9.6 Hz, 1H), 3.77 (s, 3H), 3.71˜3.65 (m, 3H), 3.60-3.52 (m, 2H), 3.44-3.37 (m, 2H), 3.17 (d, J=3.6 Hz, 3H), 2.86-2.77 (m, 1H), 2.71˜2.68 (m, 1H), 1.95-1.90 (m, 1H), 1.79-1.76 (m, 1H), 1.55-1.48 (m, 2H), 1.41 (d, J=2.4 Hz, 9H), 1.33 (dd, J=6.8 Hz, 4.0, 3H).

### Preparation of 9,10-difluoro-6-((((2-methoxypyridin-4-yl)methyl)((S)-1-(6-(methylamino)pyridin-3-yl)piperidin-3-yl)amino)methyl)-3-methyl-2H-[1,4]oxazino[2,3,4-ij]quinolin-7(3H)-one (Step 6 in Scheme 6)

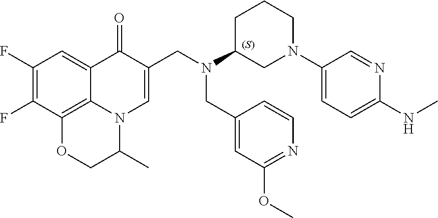

To a solution of tert-butyl N-[5-[(3S)-3-[(6,7-difluoro-2-methyl-10-oxo-4-oxa-1-azatricyclo [7.3.1.0^{5,13}]trideca-5(13),6,8,11-tetraen-11-yl)methyl-[(2-methoxy-4-pyridyl)methyl]amino]-1-piperidyl]-2-pyridyl]-N-methyl-carbamate (65 mg, 96.05 umol, 1 eq) in EtOAc (0.1 mL) was added HCl/EtOAc (4 M, 5 mL, 208.23 eq). The mixture was stirred at 20° C. for 4 hours. LCMS and HPLC indicated the reaction was complete. The reaction mixture was concentrated in vacuum. The residue was purified by prep-HPLC (column: Phenomenex C18 75*30 mm*3 um; mobile phase: [water (NH₄HCO₃)-MeCN]; B %: 25%-60%, 8 mins). The eluent was dried over lyophilization. Compound 6,7-difluoro-11-[[(2-methoxy-4-pyridyl)methyl-[(3S)-1-[6-(methylamino)-3-pyridyl]-3-piperidyl]amino]methyl]-2-methyl-4-oxa-1-azatricyclo[7.3.1.0^{5,13}]trideca-5(13),6,8,11-tetraen-10-one (5.6 mg, 9.70 umol, 10.10% yield, 99.87% purity) was obtained as white solid. ¹H NMR (DMSO-d₆, 400 MHz) δ 8.08 (d, J=7.2 Hz, 1H), 7.99 (d, J=5.2 Hz, 1H), 7.69 (s, 1H), 7.57-7.52 (m, 1H), 7.17 (d, J=9.2 Hz, 1H), 6.99 (s, 1H), 6.82 (s, 1H), 6.36 (dd, J=9.2 Hz, 3.6 Hz, 1H), 5.94 (t, J=4.8 Hz, 1H), 4.70 (d, J=5.2 Hz, 1H), 4.55 (d, J=11.2 Hz, 1H), 4.39 (d, J=11.2 Hz, 1H), 3.76 (s, 3H), 3.72˜3.58 (m, 3H), 3.56˜3.50 (m, 1H), 3.35˜3.34 (m, 1H), 3.25˜3.22 (m, 1H), 2.78˜2.73 (m, 1H), 2.70˜2.69 (m, 3H), 2.59 (t, J=10.8 Hz, 1H), 2.45-2.39 (m, 1H), 1.97 (d, J=8.4 Hz, 1H), 1.76 (d, J=11.2 Hz, 1H), 1.54-1.38 (m, 2H), 1.33 (dd, J=6.4 Hz, 2.8 Hz, 3H). LCMS: 99.87% (220 nm), 99.58% (254 nm). MS (ESI): mass calcd. For C₃₁H₃₄F₂N₆O₃ 576.27 m/z found 577.3 [M+H]⁺.

### Compound 17

### Preparation of 9,10-difluoro-6-((((2-methoxypyridin-4-yl)methyl)((S)-1-(6-(pyrrolidin-1-yl)pyridin-3-yl)piperidin-3-yl)amino)methyl)-3-methyl-2H-[1,4]oxazino[2,3,4-ij]quinolin-7(3H)-one (Step 5 in Scheme 6)

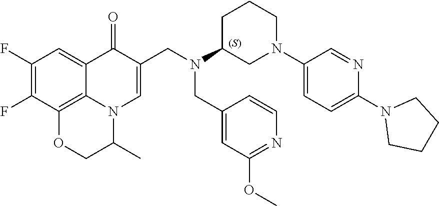

6,7-difluoro-11-[[(2-methoxy-4-pyridyl)methyl-[(3S)-1-(6-pyrrolidin-1-yl-3-pyridyl)-3-piperidyl]amino]methyl]-2-methyl-4-oxa-1-azatricyclo[7.3.1.0^{5,3}]trideca-5(13),6,8,11-tetraen-10-one (10.5 mg, 16.55 umol, 14.81% yield, 97.20% purity) was obtained as pale yellow solid. ¹H NMR (DMSO-d₆, 400 MHz) δ 8.08 (d, J=8.0 Hz, 1H), 7.99 (d, J=5.2 Hz, 1H), 7.79 (t, J=3.2 Hz, 1H), 7.56-7.51 (m, 1H), 7.27-7.23 (m, 1H), 7.01˜6.99 (m, 1H), 6.82 (s, 1H), 6.37-6.33 (m, 1H), 4.72˜4.69 (m, 1H), 4.57˜4.55 (m, 1H), 4.41˜4.38 (m, 1H), 3.78˜3.77 (m, 1H), 3.76 (s, 3H), 3.73˜3.53 (m, 4H), 3.32˜3.28 (m, 5H), 2.64˜2.58 (m, 3H), 1.99˜1.97 (m, 1H), 1.92˜1.91 (m, 4H), 1.77-1.74 (m, 1H), 1.49-1.44 (m, 2H), 1.35-1.32 (m, 3H). HPLC: 97.20% (220 nm), 99.12% (254 nm). MS (ESI): mass calcd. For C₃₄H₃₈F₂N₆O₃ 616.30 m/z found 617.3 [M+H]⁺.

### Compound 18

### Preparation of 9,10-difluoro-6-((((2-methoxypyridin-4-yl)methyl)((S)-1-(6-(2-oxopyrrolidin-1-yl)pyridin-3-yl)piperidin-3-yl)amino)methyl)-3-methyl-2H-[1,4]oxazino[2,3,4-ij]quinolin-7(3H)-one (Step 5 in Scheme 6)

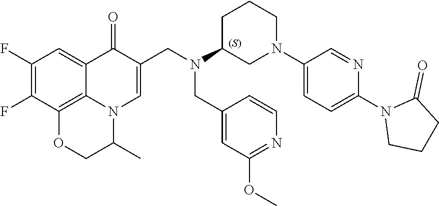

6,7-difluoro-11-[[(2-methoxy-4-pyridyl)methyl-[(3S)-1-[6-(2-oxopyrrolidin-1-yl)-3-pyridyl]-3-piperidyl]amino]methyl]-2-methyl-4-oxa-1-azatricyclo[7.3.1.0^{5,13}]trideca-5(13),6,8,11-tetraen-10-one (23.5 mg, 37.07 umol, 23.22% yield, 99.49% purity) was obtained as white solid. ¹H NMR (DMSO-d₆, 400 MHz) δ 8.12˜7.99 (m, 4H), 7.56-7.51 (m, 1H), 7.43˜7.41 (m, 1H), 7.03˜6.99 (m, 1H), 6.86 (s, 1H), 4.73˜4.69 (m, 1H), 4.58˜4.55 (m, 1H), 4.41˜4.38 (m, 1H), 3.93˜3.89 (m, 3H), 3.77 (s, 3H), 3.73˜3.51 (m, 5H), 2.79˜2.58 (m, 5H), 2.03˜1.98 (m, 3H), 1.79-1.75 (m, 1H), 1.52˜1.47 (m, 2H), 1.35-1.32 (m, 3H). HPLC: 99.49% (220 nm), 99.56% (254 nm). MS (ESI): mass calcd. For C₃₄H₃₆F₂N₆O₄ 630.28 m/z found 631.3 [M+H]⁺.

### Compound 19

### Preparation of 11-[[[(3S)-1-[6-(dimethylamino)-3-pyridyl]-3-piperidyl]-[(2-methoxy-4-pyridyl)methyl]amino]methyl]-6,7-difluoro-2-methyl-4-oxa-1-azatricyclo[7.3.1.05,13]trideca-5(13),6,8,11-tetraen-10-one (Step 5 in Scheme 6)

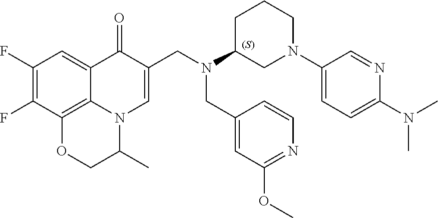

11-[[[(3S)-1-[6-(dimethylamino)-3-pyridyl]-3-piperidyl]-[(2-methoxy-4-pyridyl)methyl]amino]methyl]-6,7-difluoro-2-methyl-4-oxa-1-azatricyclo[7.3.1.05,13]trideca-5(13),6,8,11-tetraen-10-one (1.1 mg, 1.77 umol, 2.77% yield, 94.85% purity) was obtained as pale yellow solid. ¹H NMR (DMSO-d₆, 400 MHz) δ 8.08 (d, J=8.0 Hz, 1H), 7.99 (d, J=5.2 Hz, 1H), 7.82 (t, J=2.4 Hz, 1H), 7.57-7.52 (m, 1H), 7.27-7.23 (m, 1H), 7.01˜6.99 (m, 1H), 6.83˜6.82 (m, 1H), 6.56 (dd, J=6.0 Hz, 9.6 Hz, 1H), 4.72˜4.69 (m, 1H), 4.57˜4.54 (m, 1H), 4.39 (d, J=11.2 Hz, 1H), 3.78-3.77 (m, 1H), 3.76 (s, 3H), 3.72˜3.71 (m, 1H), 3.65 (d, J=14.0 Hz, 1H), 3.59˜3.58 (m, 1H), 3.57-3.54 (m, 1H), 3.29-3.27 (m, 1H), 2.92 (s, 6H), 2.78-2.72 (m, 1H), 2.68-2.65 (m, 1H), 2.62˜2.59 (m, 1H), 2.00˜1.95 (m, 1H), 1.78˜1.75 (m, 1H), 1.52˜1.43 (m, 2H), 1.33 (d, J=5.2 Hz, 3H). LCMS: 94.85% (220 nm), 96.44% (254 nm). MS (ESI): mass calcd. For C₃₂H₃₆F₂N₆O₃ 590.28 m/z found 591.3 [M+H]⁺.

### Compound 20

### Preparation of 11-[[[(3S)-1-[6-(cyclopropylmethylamino)-3-pyridyl]-3-piperidyl]-[(2-methoxy-4-pyridyl) methyl]amino]methyl]-6,7-difluoro-2-methyl-4-oxa-1-azatricyclo[7.3.1.0^{5,13}]trideca-5(13),6,8,11-tetraen-10-one (Step 5 in Scheme 6)

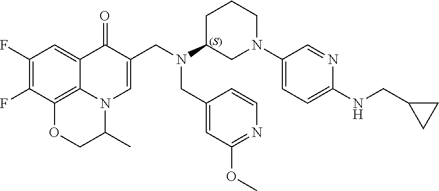

11-[[[(3S)-1-[6-(cyclopropylmethylamino)-3-pyridyl]-3-piperidyl]-[(2-methoxy-4-pyridyl) methyl]amino]methyl]-6,7-difluoro-2-methyl-4-oxa-1-azatricyclo[7.3.1.0^{5,13}]trideca-5(13),6,8,11-tetraen-10-one (10 mg, 15.38 umol, 19.28% yield, 94.87% purity) was obtained as white solid. ¹H NMR (DMSO-d₆, 400 MHz) δ 8.08-8.07 (m, 1H), 7.99 (s, 1H), 7.65 (s, 1H), 7.58-7.51 (m, 1H), 7.21˜7.11 (m, 1H), 7.00-6.99 (m, 1H), 6.82 (s, 1H), 6.42˜6.40 (m, 1H), 6.03 (s, 1H), 4.72˜4.69 (m, 1H), 4.55 (d, J=10.4 Hz, 1H), 4.42˜4.37 (m, 1H), 3.76 (s, 3H), 3.71˜3.63 (m, 2H), 3.54˜3.49 (m, 2H), 3.25˜3.17 (m, 2H), 3.03 (s, 2H), 2.79˜2.72 (m, 1H), 2.63˜2.57 (m, 2H), 1.98˜1.97 (m, 1H), 1.77˜1.75 (m, 1H), 1.50˜1.42 (m, 2H), 1.33 (s, 3H), 1.02˜0.99 (m, 1H), 0.41˜0.39 (m, 2H), 0.16 (s, 2H). LCMS: 94.87% (220 nm), 99.93% (254 nm). MS (ESI): mass calcd. For C₃₄H₃₈F₂N₆O₃ 616.30 m/z found 617.3 [M+H]⁺.

### Compound 21

### Preparation of 11-[[[(3S)-1-[6-[cyclopropyl(methyl)amino]-3-pyridyl]-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (Step 5 in Scheme 6)

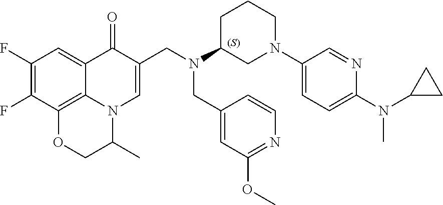

11-[[[(3S)-1-[6-[cyclopropyl(methyl)amino]-3-pyridyl]-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (9.3 mg, 14.44 umol, 9.05% yield, 95.77% purity) was obtained as white solid. ¹H NMR (DMSO-d₆, 400 MHz) δ 8.10 (d, J=8.4 Hz, 1H), 8.00 (d, J=5.6 Hz, 1H), 7.85 (t, J=2.8 Hz, 1H), 7.57-7.52 (m, 1H), 7.30-7.26 (m, 1H), 7.01˜6.99 (m, 1H), 6.90 (dd, J=7.2 Hz, 8.8 Hz, 1H), 6.83 (d, J=3.2 Hz, 1H), 4.71˜4.69 (m, 1H), 4.57 (d, J=11.2 Hz, 1H), 4.40 (d, J=10.8 Hz, 1H), 3.82 (s, 1H), 3.76 (s, 3H), 3.72˜3.71 (m, 1H), 3.69˜3.62 (m, 2H), 3.60-3.54 (m, 2H), 3.34 (s, 1H), 2.97 (d, J=2.4 Hz, 3H), 2.76-2.73 (m, 1H), 2.67-2.61 (m, 1H), 2.37-2.33 (m, 1H), 1.99 (d, J=9.6 Hz, 1H), 1.78 (d, J=10.8 Hz, 1H), 1.51˜1.45 (m, 2H), 1.34 (d, J=6.8 Hz, 3H), 0.83˜0.78 (m, 2H), 0.53˜0.52 (m, 2H). MS: 95.77% (220 nm), 97.99% (254 nm). MS (ESI): mass calcd. For C₃₄H₃₈F₂N₆O₃ 616.30 m/z found 617.4 [M+H]⁺.

### Compound 22

### Preparation of 9,10-difluoro-6-((((2-methoxypyridin-4-yl)methyl)((S)-1-(pyridin-3-yl)piperidin-3-yl)amino)methyl)-3-methyl-2H-[1,4]oxazino[2,3,4-ij]quinolin-7(3H)-one (Step 5 in Scheme 6)

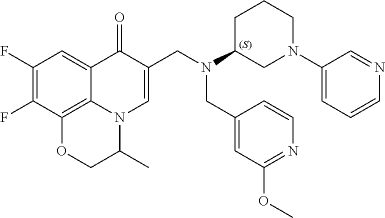

Compound 9,10-difluoro-6-((((2-methoxypyridin-4-yl)methyl)((S)-1-(pyridin-3-yl)piperidin-3-yl)amino)methyl)-3-methyl-2H-[1,4]oxazino[2,3,4-ij]quinolin-7(3H)-one (3.2 mg, 5.84 umol, 3.66% yield, 100% purity) was obtained as white solid. ¹H NMR (DMSO-d₆, 400 MHz) δ 8.30 (d, J=2.8 Hz, 1H), 8.13 (d, J=6.0 Hz, 1H), 8.01 (d, J=5.2 Hz, 1H), 7.92 (t, J=4.4 Hz, 1H), 7.57-7.52 (m, 1H), 7.30-7.27 (m, 1H), 7.17-7.13 (m, 1H), 7.03 (d, J=4.8 Hz, 1H), 6.86 (d, J=4.4 Hz, 1H), 4.74˜4.67 (m, 1H), 4.58 (d, J=11.6 Hz, 1H), 4.41 (d, J=11.6 Hz, 1H), 3.95-3.92 (m, 1H), 3.84˜3.80 (m, 1H), 3.77 (s, 3H), 3.74 (s, 1H), 3.72˜3.70 (m, 1H), 3.68˜3.66 (m, 1H), 3.61˜3.54 (m, 1H), 2.86-2.79 (m, 1H), 2.77-2.60 (m, 2H), 2.02 (d, J=10.0 Hz, 1H), 1.79-1.76 (m, 1H), 1.57-1.44 (m, 2H), 1.33 (t, J=8.0 Hz, 3H). MS: 100.00% (220 nm), 100% (254 nm). MS (ESI): mass calcd. For C₃₀H₃₁F₂N₅O₃ 547.27 m/z found 548.1 [M+H]⁺.

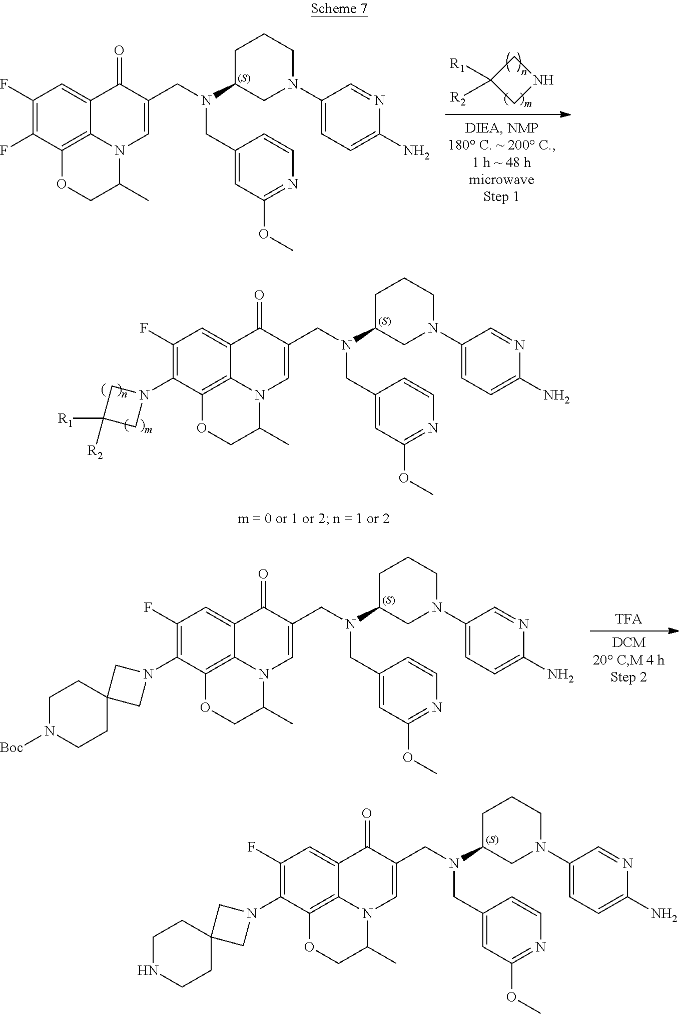

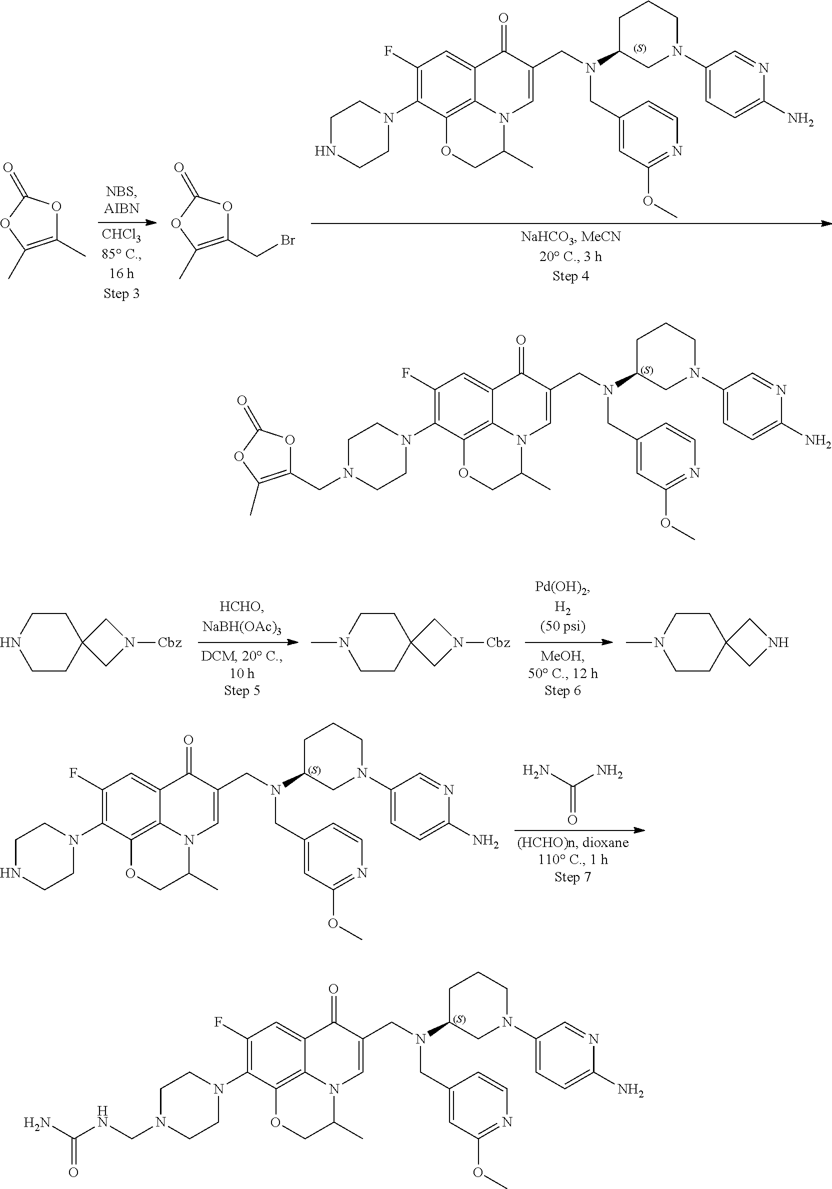

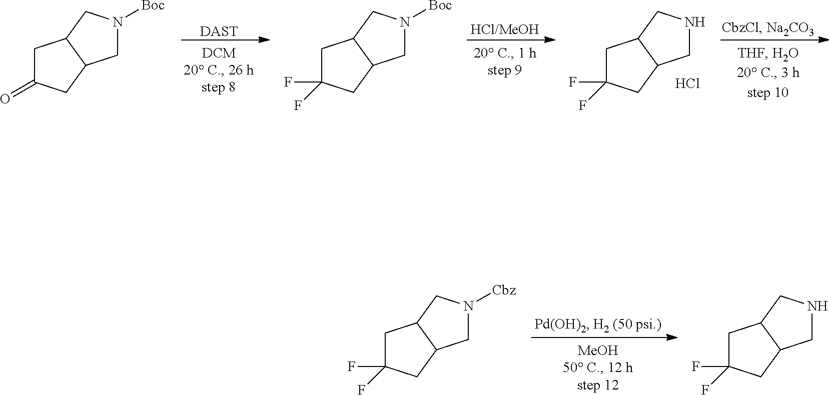

### General Procedures for Preparing Compounds in Scheme 7

### Preparation of Compounds in Scheme 7 (Step 1 in Scheme 7)

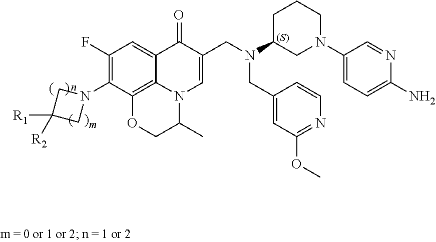

To a mixture of 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (710.97 umol, 1 eq) and different amine (2.13 mmol˜14.22 mmol, 3 eq˜20 eq) in NMP (3 mL/mmol˜5 mL/mmol) was added DIEA (2.13 mmol˜5.68 mmol, 3 eq˜8 eq). The mixture was stirred at 180° C.˜220° C. for an hour˜48 hours under microwave. The reaction mixture was quenched with ice water at 0° C. and then extracted with EtOAc. The combined organic layers were washed with brine, dried over anhydrous Na₂SO₄, filtered and concentrated under reduced pressure. The residue was purified by flash silica gel chromatography. The eluent was concentrated in vacuum. Or the reaction mixture was filtered and the filtrate was directly purified by prep-HPLC (column: Waters Xbridge Prep OBD C 18 150*40 mm*10 um or Waters Xbridge BEH C18 100*30 mm*10 um or Phenomenex C18 75*30 mm*3 um or Phenomenex C 18 80*40 mm*3 um or Phenomenex Luna 80*30 mm*3 um; mobile phase: [water (NH₄HCO₃)-MeCN] or [water (HCl)-MeCN]; B %: 1%-75%, 7 mins or 8 mins or 10 mins). The eluent was removed under freeze drying to give the desired product.

### Compound 24

### Preparation of tert-butyl 2-(6-((((S)-1-(6-aminopyridin-3-yl)piperidin-3-yl)((2-methoxypyridin-4-yl)methyl)amino)methyl)-9-fluoro-3-methyl-7-oxo-3,7-dihydro-2H-[1,4]oxazino[2,3,4-ij]quinolin-10-yl)-2,7-diazaspiro[3.5]nonane-7-carboxylate (Step 1 in Scheme 7)

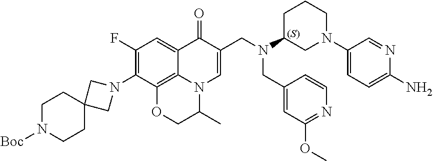

To a mixture of 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one (400 mg, 710.97 umol, 1 eq) and tert-butyl 2,7-diazaspiro[3.5]nonane-7-carboxylate (482.71 mg, 2.13 mmol, 3 eq) in NMP (2.5 mL) was added DIEA (275.66 mg, 2.13 mmol, 371.51 uL, 3 eq). The mixture was stirred at 190° C. for an hour under microwave. LCMS indicated 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo[7.3.1.0^{5,13}]trideca-5(13),6,8,11-tetraen-10-one was consumed and one new peak with desired mass was detected. The reaction mixture was quenched with ice water (15 mL) at 0° C. and then extracted with EtOAc (15 mL×2). The combined organic layers were washed with brine (15 mL×2), dried over anhydrous Na₂SO₄, filtered and concentrated under reduced pressure. The residue was purified by flash silica gel chromatography (Biotage®; 12 g SepaFlash® Silica Flash Column, Eluent of 0˜100% Ethyl acetate/Petroleum ether gradient @40 mL/min). The eluent was concentrated in vacuum. Compound tert-butyl 2-[11-[[[(3S)-1-(6-amino-3-pyridyl)-3-piperidyl]-[(2-methoxy-4-pyridyl) methyl]amino]methyl]-7-fluoro-2-methyl-10-oxo-4-oxa-1-azatricyclo[7.3.1.0^{5,13}]trideca-5(13),6,8,11-tetraen-6-yl]-2,7-diazaspiro[3.5]nonane-7-carboxylate (0.4 g, 520.21 umol, 73.17% yield) was obtained as purple solid. ¹H NMR (DMSO-d₆, 400 MHz) δ 7.99 (d, J=5.2 Hz, 1H), 7.85 (d, J=7.6 Hz, 1H), 7.60 (d, J=2.8 Hz, 1H), 7.27 (dd, J=2.4 Hz, 14.0 Hz, 1H), 7.15-7.12 (m, 1H), 6.99 (d, J=4.8 Hz, 1H), 6.82 (s, 1H), 6.37 (dd, J=3.2 Hz, 8.8 Hz, 1H), 5.37 (s, 2H), 4.51˜4.49 (m, 1H), 4.33 (d, J=10.4 Hz, 1H), 4.13 (d, J=9.2 Hz, 1H), 4.02˜4.00 (m, 4H), 3.77 (s, 3H), 3.70˜3.69 (m, 1H), 3.65˜3.54 (m, 2H), 3.51˜3.46 (m, 1H), 3.32˜3.28 (m, 5H), 3.24-3.22 (m, 1H), 2.76-2.72 (m, 1H), 2.59-2.56 (m, 1H), 2.42˜2.37 (m, 1H), 1.96-1.92 (m, 1H), 1.76-1.73 (m, 1H), 1.69-1.67 (m, 4H), 1.39-1.37 (m, 11H), 1.29 (dd, J=4.8 Hz, 6.0 Hz, 3H).

### Preparation of 6-((((S)-1-(6-aminopyridin-3-yl)piperidin-3-yl)((2-methoxypyridin-4-yl)methyl)amino)methyl)-9-fluoro-3-methyl-10-(2,7-diazaspiro[3.5]nonan-2-yl)-2H-[1,4]oxazino[2,3,4-ij]quinolin-7(3H)-one (Step 2 in Scheme 7)

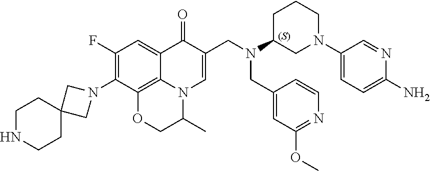

To a solution of tert-butyl 2-[11-[[[(3S)-1-(6-amino-3-pyridyl)-3-piperidyl]-[(2-methoxy-4-pyridyl)methyl]amino]methyl]-7-fluoro-2-methyl-10-oxo-4-oxa-1-azatricyclo[7.3.1.0^{5,13}]trideca-5(13),6,8,11-tetraen-6-yl]-2,7-diazaspiro[3.5]nonane-7-carboxylate (300 mg, 390.16 umol, 1 eq) in DCM (3 mL) was added TFA (1.54 g, 13.51 mmol, 1 mL, 34.62 eq). The mixture was stirred at 25° C. for 4 hours. LCMS and HPLC indicated the reaction was complete. The reaction mixture was quenched by addition H₂O (2 mL) at 0° C. The reaction mixture was adjusted to pH=7 with sat. Na₂CO₃ and dried over lyophilization. The residue was washed with MeCN (20 mL). The filtrate was concentrated under reduced pressure to give crude 11-[[[(3S)-1-(6-amino-3-pyridyl)-3-piperidyl]-[(2-methoxy-4-pyridyl)methyl]amino]methyl]-6-(2,7-diazaspiro[3.5]nonan-2-yl)-7-fluoro-2-methyl-4-oxa-1-azatricyclo[7.3.1.0^{5,13}]trideca-5(13),6,8,11-tetraen-10-one (200 mg, crude) as purple solid. 60 mg of the residue was further purified by prep-HPLC (column: Phenomenex Luna C18 75*30 mm*3 um; mobile phase: [water (NH₄HCO₃)-MeCN]; B %: 10%-40%, 8 mins). The eluent was directly dried over lyophilization. Compound 11-[[[(3S)-1-(6-amino-3-pyridyl)-3-piperidyl]-[(2-methoxy-4-pyridyl)methyl]amino]methyl]-6-(2,7-diazaspiro[3.5]nonan-2-yl)-7-fluoro-2-methyl-4-oxa-1-azatricyclo[7.3.1.0^{5,13}]trideca-5(13),6,8,11-tetraen-10-one (26.1 mg, 37.84 umol, 9.70% yield, 96.962% purity) was obtained as gray solid. ¹H NMR (DMSO-d₆, 400 MHz) δ 7.99 (d, J=5.2 Hz, 1H), 7.85 (d, J=7.6 Hz, 1H), 7.60 (d, J=2.8 Hz, 1H), 7.26 (dd, J=2.0 Hz, 13.6 Hz, 1H), 7.15-7.12 (m, 1H), 6.99 (d, J=4.8 Hz, 1H), 6.81 (s, 1H), 6.37 (dd, J=3.2 Hz, 8.8 Hz, 1H), 5.36 (d, J=3.2 Hz, 2H), 4.52˜4.32 (m, 1H), 4.33 (d, J=10.0 Hz, 1H), 4.13 (d, J=9.2 Hz, 1H), 3.96 (d, J=2.8 Hz, 4H), 3.77 (s, 3H), 3.74-3.65 (m, 2H), 3.63˜3.54 (m, 2H), 3.51˜3.46 (m, 1H), 3.24-3.20 (m, 1H), 2.76-2.71 (m, 1H), 2.64-2.58 (m, 4H), 2.56-2.55 (m, 1H), 2.43˜2.37 (m, 1H), 1.96-1.94 (m, 1H), 1.76-1.73 (m, 1H), 1.67-1.60 (m, 4H), 1.49-1.37 (m, 2H), 1.30-1.28 (m, 3H). LCMS: 96.96% (220 nm), 98.37% (254 nm). MS (ESI): mass calcd. For C₃₇H₄₅FN₈O₃ 668.36 m/z found 669.5 [M+H]⁺.

### Compound 25

### Preparation of 4-(bromomethyl)-5-methyl-1,3-dioxol-2-one (Step 3 in Scheme 7)

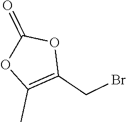

To a solution of 4,5-dimethyl-1,3-dioxol-2-one (5 g, 43.82 mmol, 1 eq) in CHCl₃ (100 mL) was added NBS (7.80 g, 43.82 mmol, 1 eq) and AIBN (359.79 mg, 2.19 mmol, 0.05 eq) at 20° C. under N₂. The mixture was stirred at 85° C. for 16 hours. TLC (Petroleum ether:Ethyl acetate=1:1, R_{f}=0.46) showed 4,5-dimethyl-1,3-dioxol-2-one was consumed completely and one new spot formed. The reaction mixture was cooled to 20° C. and quenched by addition water (200 mL), then extracted with EtOAc (200 mL×3). The combined organic layers were washed with brine (150 mL), dried over Na₂SO₄ filtered and concentrated under reduced pressure. The residue was purified by flash silica gel chromatography (Biotage®; 80 g SepaFlash® Silica Flash Column, Eluent of 0˜50% gradient ethyl acetate/petroleum ether @100 mL/min). The solvent was removed under reduced pressure. Compound 4-(bromomethyl)-5-methyl-1,3-dioxol-2-one (7.6 g, 39.38 mmol, 89.86% yield) was obtained as brown oil. ¹H NMR (DMSO-d₆, 400 MHz) δ 4.68 (s, 2H), 2.15 (s, 3H)

### Preparation of 6-((((S)-1-(6-aminopyridin-3-yl)piperidin-3-yl)((2-methoxypyridin-4-yl)methyl)amino)methyl)-9-fluoro-3-methyl-10-(4-((5-methyl-2-oxo-1,3-dioxol-4-yl)methyl)piperazin-1-yl)-2H-[1,4]oxazino[2,3,4-ij]quinolin-7(3H)-one (Step 4 in Scheme 7)

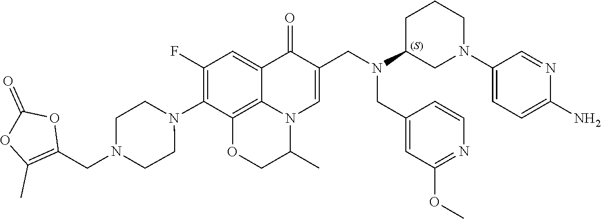

To a mixture of 4-(bromomethyl)-5-methyl-1,3-dioxol-2-one (30.70 mg, 159.05 umol, 1 eq) and 11-[[[(3S)-1-(6-amino-3-pyridyl)-3-piperidyl]-[(2-methoxy-4-pyridyl)methyl]amino]methyl]-7-fluoro-2-methyl-6-piperazin-1-yl-4-oxa-1-azatricyclo[7.3.1.05,13]trideca-5(13),6,8,11-tetraen-10-one (100 mg, 159.05 umol, 1 eq) in MeCN (0.5 mL) was added NaHCO₃ (40.08 mg, 477.15 umol, 18.56 uL, 3 eq) at 20° C. under N₂. The reaction mixture was stirred at 20° C. for 3 hours. LC-MS showed 4-(bromomethyl)-5-methyl-1,3-dioxol-2-one was consumed completely and one new peak with desired mass was detected. The reaction mixture was filtered and the filtrate was concentrated under reduced pressure. The residue was purified by prep-HPLC (column: Waters Xbridge BEH C 18 100*30 mm*10 um; mobile phase: [water (NH₄HCO₃)-MeCN]; B %: 35%-65%, 8 mins). The solvent was removed under freeze drying. Compound 11-[[[(3S)-1-(6-amino-3-pyridyl)-3-piperidyl]-[(2-methoxy-4-pyridyl)methyl]amino]methyl]-7-fluoro-2-methyl-6-[4-[(5-methyl-2-oxo-1,3-dioxol-4-yl)methyl]piperazin-1-yl]-4-oxa-1-azatricyclo[7.3.1.05,13]trideca-5(13),6,8,11-tetraen-10-one (6.3 mg, 8.50 umol, 5.35% yield, 100% purity) was obtained as white solid. ¹H NMR (DMSO-d₆, 400 MHz) δ 8.00-7.95 (m, 2H), 7.60 (d, J=2.4 Hz, 1H), 7.34 (dd, J=2.4 Hz, 12.8 Hz, 1H), 7.14 (dt, J=2.8 Hz, 8.8 Hz, 1H), 7.00 (d, J=4.8 Hz, 1H), 6.81 (s, 1H), 6.39 (dd, J=3.6 Hz, 8.8 Hz, 1H), 5.37 (d, J=3.6 Hz, 2H), 4.58˜4.46 (m, 1H), 4.43 (d, J=10.0 Hz, 1H), 4.26 (dd, J=2.8 Hz, =12.0 Hz, 1H), 3.76 (s, 4H), 3.71˜3.70 (m, 1H), 3.61˜3.60 (m, 1H), 3.58 (s, 1H), 3.50˜3.47 (m, 1H), 3.43 (s, 2H), 3.21 (s, 4H), 3.12˜3.10 (m, 1H), 2.80˜2.71 (m, 2H), 2.57˜2.54 (m, 4H), 2.43˜2.38 (m, 1H), 2.12 (s, 3H), 1.97˜1.93 (m, 1H), 1.78-1.72 (m, 1H), 1.50-1.40 (m, 2H), 1.31 (t, J=4.8 Hz, 3H). LCMS: 100.00% (220 nm), 100.00% (254 nm). MS (ESI): mass calcd. For C₃₉H₄₅FN₈O₆ 740.34 m/z found 741.1 [M+H]⁺.

### Preparation of benzyl 7-methyl-2,7-diazaspiro[3.5]nonane-2-carboxylate (Step 5 in Scheme 7)

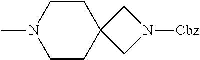 [SMILES: CN1CCC2(CC1)CN(C(=O)OCc1ccccc1)C2]

To a solution of benzyl 2,7-diazaspiro[3.5]nonane-2-carboxylate (0.5 g, 1.92 mmol, 1 eq) and formaldehyde (311.72 mg, 3.84 mmol, 285.98 uL, 37% purity, 2 eq) in DCM (5 mL) was added NaBH(OAc)₃ (610.59 mg, 2.88 mmol, 1.5 eq). The reaction mixture was stirred at 20° C. for 10 hours. LCMS showed the reaction was complete. The reaction mixture was quenched with ice water (40 mL) and then adjusted to pH=7 with sat. NaHCO₃. The mixture was extracted with DCM (15 mL×3). The combined organic phase was washed with brine (15 mL×2), dried with anhydrous Na₂SO₄, filtered and concentrated in vacuum. Compound benzyl 7-methyl-2,7-diazaspiro[3.5]nonane-2-carboxylate (0.5 g, 1.82 mmol, 94.89% yield) was obtained as colorless oil. ¹H NMR (DMSO-d₆, 400 MHz) δ 7.39-7.31 (m, 5H), 5.02 (s, 2H), 3.62 (d, J=2.0 Hz, 4H), 2.21 (s, 4H), 2.12 (s, 3H), 1.66 (t, J=5.2 Hz, 4H).

### Preparation of 7-methyl-2,7-diazaspiro[3.5]nonane (Step 6 in Scheme 7)

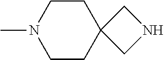

A mixture of benzyl 7-methyl-2,7-diazaspiro[3.5]nonane-2-carboxylate (0.5 g, 1.82 mmol, 1 eq) and Pd(OH)₂ (0.1 g, 10% purity) in MeOH (30 mL) was degassed and purged with H₂ for 3 times, and then the mixture was stirred at 50° C. for 12 hours under H₂ (50 psi.) atmosphere. LCMS showed the reaction was complete. The reaction mixture was filtered through a pad of the Celite and the filter cake was washed with MeOH (60 mL). The combined filtrate was concentrated under reduced pressure. Compound 7-methyl-2,7-diazaspiro[3.5]nonane (0.25 g, crude) was obtained as colorless oil. ¹H NMR (DMSO-d₆, 400 MHz) δ 3.61˜3.58 (m, 1H), 3.51˜3.47 (m, 1H), 3.21˜3.17 (m, 3H), 3.19˜3.15 (m, 3H), 2.08 (s, 3H), 2.03˜1.99 (m, 1H), 1.74-1.59 (m, 4H).

### Compound 26

### Preparation of 1-((4-(6-((((S)-1-(6-aminopyridin-3-yl)piperidin-3-yl)((2-methoxypyridin-4-yl)methyl)amino)methyl)-9-fluoro-3-methyl-7-oxo-3,7-dihydro-2H-[1,4]oxazino[2,3,4-ij]quinolin-10-yl)piperazin-1-yl)methyl)urea (Step 7 in Scheme 7)

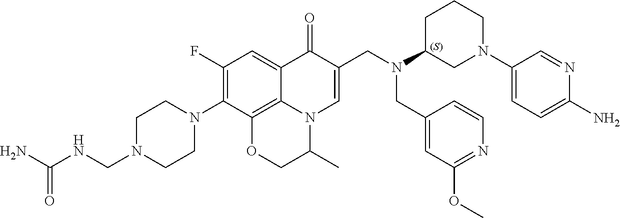

To a solution of 11-[[[(3S)-1-(6-amino-3-pyridyl)-3-piperidyl]-[(2-methoxy-4-pyridyl)methyl]amino]methyl]-7-fluoro-2-methyl-6-piperazin-1-yl-4-oxa-1-azatricyclo[7.3.1.0^{5,13}]trideca-5(13),6,8,11-tetraen-10-one (100 mg, 159.05 umol, 1 eq) in dioxane (4 mL) was added (HCHO)n (14.31 mg, 477.15 umol, 13.13 uL, 3 eq) and urea (14.33 mg, 238.57 umol, 12.79 uL, 1.5 eq). The mixture was stirred at 110° C. for an hour. LCMS and HPLC indicated the reaction was completed. The reaction mixture was filtered and the filtrate was concentrated under reduced pressure. The residue was purified by prep-HPLC (column: Waters Xbridge BEH C18 100*30 mm*10 um; mobile phase: [water (NH₄HCO₃)-MeCN]; B %: 20%-40%, 8 mins). The eluent was dried over lyophilization. The obtained crude product was further purified by prep-HPLC (column: Waters Xbridge BEH C18 100*30 mm*10 um; mobile phase: [water (NH₄HCO₃)-MeCN]; B %: 20%-40%, 8 mins). The eluent was dried over lyophilization. Compound [4-[11-[[[(3S)-1-(6-amino-3-pyridyl)-3-piperidyl]-[(2-methoxy-4-pyridyl)methyl]amino]methyl]-7-fluoro-2-methyl-10-oxo-4-oxa-1-azatricyclo[7.3.1.0^{5,13}]trideca-5(13),6,8,11-tetraen-6-yl]piperazin-1-yl]methylurea (1.6 mg, 2.28 umol, 1.43% yield, 99.80% purity) was obtained as white solid. ¹H NMR (DMSO-d₆, 400 MHz) δ 7.99 (d, J=5.2 Hz, 1H), 7.95 (d, J=6.8 Hz, 1H), 7.77 (t, J=2.8 Hz, 1H), 7.33 (dd, J=1.6 Hz, 12.4 Hz, 1H), 7.22˜7.19 (m, 1H), 6.99 (d, J=4.8 Hz, 1H), 6.81 (s, 1H), 6.61˜6.57 (m, 1H), 6.48 (dd, J=3.2 Hz, 9.2 Hz, 1H), 6.40 (t, J=6.0 Hz, 1H), 5.57 (s, 2H), 4.57-4.55 (m, 1H), 4.49˜4.43 (m, 3H), 4.26-4.24 (m, 1H), 3.78-3.75 (m, 4H), 3.71 (m, 1H), 3.66˜3.58 (m, 2H), 3.55˜3.51 (m, 1H), 3.27 (m, 1H), 3.14˜3.07 (m, 4H), 2.79-2.77 (m, 4H), 2.74-2.72 (m, 1H), 2.61˜2.56 (m, 1H), 2.45-2.39 (m, 1H), 1.97-1.95 (m, 1H), 1.78-1.175 (m, 1H), 1.55-1.38 (m, 2H), 1.31 (dd, J=4.0 Hz, 6.4 Hz, 3H). LCMS: 99.80% (220 nm), 100% (254 nm). MS (ESI): mass calcd. For C₃₆H₄₅FN₁₀O₄ 700.36 m/z found 701.5 [M+H]⁺.

### Preparation of tert-butyl 5,5-difluorohexahydrocyclopenta[c]pyrrole-2(1H)-carboxylate (Step 8 in Scheme 7)

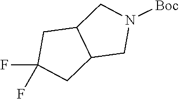 [SMILES: CC(C)(C)OC(=O)N1CC2CC(F)(F)CC2C1]

To a solution of tert-butyl 5-oxo-1,3,3a,4,6,6a-hexahydrocyclopenta[c]pyrrole-2-carboxylate (2 g, 8.88 mmol, 1 eq) in DCM (20 mL) was added DAST (7.15 g, 44.39 mmol, 5.86 mL, 5 eq) dropwise at 0° C. under N₂. The mixture was stirred at 20° C. for 12 hours. Then DAST (4.29 g, 26.63 mmol, 3.52 mL, 3 eq) was added dropwise at 0° C. under N₂ and stirred at 20° C. for 24 hours. TLC (Petroleum ether:Ethyl acetate=3:1, R_{f}=0.56) showed the reaction was nearly complete. The mixture was quenched with ice water (50 mL) at 0° C. and adjusted pH=7 with sat. NaHCO₃. The mixture was extracted with DCM (20 mL×3). The combined organic layer was washed with brine (20 mL×1), dried over Na₂SO₄, filtered and concentrated under reduced pressure. The residue was purified by flash silica gel chromatography (Biotage®; 20 g SepaFlash® Silica Flash Column, Eluent of 0˜24% petroleum ether/Ethyl acetate gradient @40 mL/min.). The eluent was removed under reduced pressure. Compound tert-butyl 5,5-difluoro-1,3,3a,4,6,6a-hexahydrocyclopenta [c]pyrrole-2-carboxylate (1.4 g, 5.66 mmol, 63.77% yield) was obtained as yellow solid. ¹H NMR (DMSO-d₆, 400 MHz) δ 3.57-3.50 (m, 2H), 3.28-3.27 (m, 2H), 2.84-2.78 (m, 2H), 2.38-2.34 (m, 2H), 2.07-1.98 (m, 2H), 1.47 (s, 9H).

### Preparation of 5,5-difluorooctahydrocyclopenta[c]pyrrole hydrochloride (Step 9 in Scheme 7)

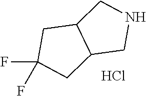

To a solution of tert-butyl 5,5-difluoro-1,3,3a,4,6,6a-hexahydrocyclopenta[c]pyrrole-2-carboxylate (0.9 g, 3.64 mmol, 1 eq) in MeOH (1 mL) was added HCl/MeOH (4 M, 9.00 mL, 9.89 eq). The mixture was stirred at 20° C. for an hour. LCMS showed the reaction was complete. The mixture was concentrated under reduced pressure and then adjusted pH=7 with sat. NaHCO₃. The eluent was removed under freeze drying. The residue was washed with DCM (150 mL), and the filtrate was concentrated under reduced pressure. Compound 5,5-difluoro-2,3,3a,4,6,6a-hexahydro-1H-cyclopenta[c]pyrrole (0.56 g, crude) was obtained as brown solid. ¹H NMR (CDCl₃, 400 MHz) δ 8.97-8.75 (m, 1H), 3.54-3.50 (m, 2H), 3.19-3.15 (m, 4H), 2.39-2.31 (m, 2H), 2.26-2.18 (m, 2H).

### Preparation of benzyl 5,5-difluorohexahydrocyclopenta[c]pyrrole-2(1H)-carboxylate (Step 10 in Scheme 7

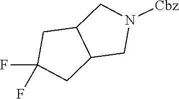 [SMILES: O=C(OCc1ccccc1)N1CC2CC(F)(F)CC2C1]

To a solution of 5,5-difluoro-2,3,3a,4,6,6a-hexahydro-1H-cyclopenta[c]pyrrole (0.3 g, 2.04 mmol, 1 eq) in THF (2 mL) and H₂O (1 mL) was added Na₂CO₃ (432.12 mg, 4.08 mmol, 2 eq) Then CbzCl (347.76 mg, 2.04 mmol, 289.80 uL, 1 eq) was added dropwise at 0° C. The resulting mixture was stirred at 20° C. for 3 hours. LCMS showed the reaction was complete. The reaction mixture was quenched with ice water (40 mL) and then extracted with EtOAc (20 mL×3). The combined organic layer was washed with brine (20 mL×1), dried over Na₂SO₄, filtered and concentrated under reduced pressure. The residue was purified by flash silica gel chromatography (Biotage®; 12 g SepaFlash® Silica Flash Column, Eluent of 0˜26% petroleum ether/Ethyl acetate gradient @40 mL/min.). The eluent was removed under reduced pressure. Compound benzyl 5,5-difluoro-1,3,3a,4,6,6a-hexahydrocyclopenta[c]pyrrole-2-carboxylate (0.38 g, 1.35 mmol, 66.27% yield) was obtained as yellow oil. ¹H NMR (DMSO-d₆, 400 MHz) δ 7.37˜7.31 (m, 5H), 5.06 (s, 2H), 3.57˜3.55 (m, 2H), 3.26˜3.23 (m, 2H), 2.81˜2.80 (m, 2H), 2.34-2.26 (m, 2H), 2.07-1.96 (m, 2H).

### Preparation of 5,5-difluorooctahydrocyclopenta[c]pyrrole (Step 11 in Scheme 7)

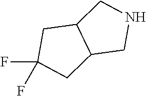

A mixture of benzyl 5,5-difluoro-1, 3,3a,4,6,6a-hexahydrocyclopenta[c]pyrrole-2-carboxylate (0.4 g, 1.42 mmol, 1 eq), Pd(OH)₂ (80 mg, 20% purity) in MeOH (50 mL) was degassed and purged with H₂ for 3 times. Then the mixture was stirred at 50° C. for 12 hours under H₂ (50 psi) atmosphere. LCMS showed the reaction was complete. The reaction mixture was filtered through a pad of the Celite and the filter cake was washed with MeOH (200 mL). The combined filtrate was concentrated under reduced pressure. Compound 5,5-difluoro-2,3,3a,4,6,6a-hexahydro-1H-cyclopenta[c]pyrrole (0.12 g, 815.41 umol, 57.34% yield) was obtained as white solid. ¹H NMR (MeOD, 400 MHz) δ 3.47-3.45 (m, 2H), 3.09-3.06 (m, 4H), 2.42˜2.37 (m, 2H), 2.11˜2.02 (m, 2H).

### Compound 27

### 6-((((S)-1-(6-aminopyridin-3-yl)piperidin-3-yl)((2-methoxypyridin-4-yl)methyl)amino)methyl)-9-fluoro-3-methyl-10-morpholino-2H-[1,4]oxazino[2,3,4-ij]quinolin-7(3H)-one was Prepared According to the Procedure Described Herein for Step 1 in Scheme 7

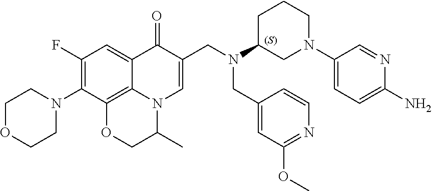

11-[[[(3S)-1-(6-amino-3-pyridyl)-3-piperidyl]-[(2-methoxy-4-pyridyl)methyl]amino]methyl]-7-fluoro-2-methyl-6-morpholino-4-oxa-1-azatricyclo[7.3.1.0^{5,13}]trideca-5(13),6,8,11-tetraen-10-one (8 mg, 12.49 umol, 17.57% yield, 98.34% purity) was obtained as off white solid. ¹H NMR (DMSO-d₆, 400 MHz) δ 8.00-7.96 (m, 2H), 7.60 (s, 1H), 7.35 (d, J=2.0 Hz, 12.4 Hz, 1H), 7.15˜7.12 (m, 1H), 7.00˜6.98 (m, 1H), 6.81 (s, 1H), 6.39˜6.35 (m, 1H), 5.37 (s, 2H), 4.60˜4.58 (m, 1H), 4.47˜4.45 (m, 1H), 4.28˜4.26 (m, 1H), 3.76 (s, 3H), 3.73˜3.39 (m, 9H), 3.30˜3.19 (m, 5H), 2.70-2.67 (m, 1H), 2.57-2.52 (m, 1H), 2.40-2.37 (m, 1H), 1.99-1.91 (m, 1H), 1.76-1.72 (m, 1H), 1.39-1.31 (m, 2H), 1.31˜1.29 (m, 3H). HPLC: 98.34% (220 nm), 98.74% (254 nm). MS (ESI): mass calcd. For C₃₄H₄₀FN₇O₄ 629.31 m/z found 630.3 [M+H]⁺.

### Compound 28

### 6-((((S)-1-(6-aminopyridin-3-yl)piperidin-3-yl)((2-methoxypyridin-4-yl)methyl)amino)methyl)-9-fluoro-3-methyl-10-(piperidin-1-yl)-2H-[1,4]oxazino[2,3,4-ij]quinolin-7(3H)-one was Prepared According to the Procedure Described Herein for Step 1 in Scheme 7

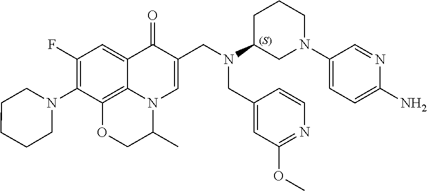

11-[[[(3S)-1-(6-amino-3-pyridyl)-3-piperidyl]-[(2-methoxy-4-pyridyl)methyl]amino]methyl]-7-fluoro-2-methyl-6-(1-piperidyl)-4-oxa-1-azatricyclo[7.3.1.05,13]trideca-5(13),6,8,11-tetraen-10-one (8.8 mg, 14.02 umol, 5.26% yield) was obtained as white solid. ¹H NMR (DMSO-d₆, 400 MHz) δ 7.99 (d, J=5.2 Hz, 1H), 7.96 (d, J=8.0 Hz, 1H), 7.60 (d, J=2.8 Hz, 1H), 7.32 (dd, J=12.4 Hz, 2.4 Hz, 1H), 7.16-7.12 (m, 1H), 6.99 (d, J=5.2 Hz, 1H), 6.81 (s, 1H), 6.37 (dd, J=8.8 Hz, 3.6 Hz, 1H), 5.37 (d, J=3.6 Hz, 2H), 4.59˜4.57 (m, 1H), 4.44 (d, J=11.6 Hz, 1H), 4.25 (dd, J=12.0 Hz, 2.4 Hz, 1H), 3.76 (s, 3H), 3.70 (d, J=4.8 Hz, 1H), 3.65-3.53 (m, 3H), 3.50-3.46 (m, 1H), 3.24-3.21 (m, 1H), 3.16-3.14 (m, 4H), 2.77-2.70 (m, 1H), 2.58 (d, J=10.4 Hz, 1H), 2.44˜2.37 (m, 1H), 1.99˜1.95 (m, 1H), 1.77˜1.73 (m, 1H), 1.59˜1.57 (m, 6H), 1.49-1.39 (m, 2H), 1.31 (t, J=5.2 Hz, 3H). LCMS: 100.00% (220 nm), 100.00% (254 nm). MS (ESI): mass calcd. For C₃₅H₄₂FN₇O₃ 627.33 m/z found 628.4 [M+H]⁺.

### Compound 29

### 6-((((S)-1-(6-aminopyridin-3-yl)piperidin-3-yl)((2-methoxypyridin-4-yl)methyl)amino)methyl)-9-fluoro-3-methyl-10-(4-methylpiperazin-1-yl)-2H-[1,4]oxazino[2,3,4-ij]quinolin-7(3H)-one was Prepared According to the Procedure Described Herein for Step 1 in Scheme 7

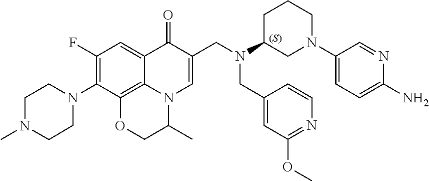

11-[[[(3S)-1-(6-amino-3-pyridyl)-3-piperidyl]-[(2-methoxy-4-pyridyl)methyl]amino]methyl]-7-fluoro-2-methyl-6-(4-methylpiperazin-1-yl)-4-oxa-1-azatricyclo[7.3.1.0^{5,13}]trideca-5(13),6,8,11-tetraen-10-one (13.1 mg, 20.38 umol, 14.33% yield, 100% purity) was obtained as white solid. ¹H NMR (DMSO-d₆, 400 MHz) δ 8.02˜7.97 (m, 2H), 7.64-7.58 (m, 1H), 7.37-7.31 (m, 1H), 7.17-7.11 (m, 1H), 7.02˜6.97 (m, 1H), 6.84-6.79 (m, 1H), 6.40-6.35 (m, 1H), 5.41˜5.31 (m, 2H), 4.59-4.53 (m, 1H), 4.48˜4.39 (m, 1H), 4.30˜4.20 (m, 1H), 3.76 (s, 3H), 3.72˜3.70 (m, 1H), 3.62˜3.58 (m, 2H), 3.52˜3.46 (m, 1H), 3.31 (s, 2H), 3.19 (s, 4H), 2.80˜2.60 (m, 3H), 2.41 (s, 4H), 2.22 (s, 3H), 2.01˜1.92 (m, 1H), 1.77˜1.73 (m, 1H), 1.50˜1.42 (m, 2H), 1.31 (s, 3H). LCMS: 100% (220 nm), 100% (254 nm). MS (ESI): mass calcd. For C₃₅H₄₃FN₈O₃ 642.34 m/z found 643.4 [M+H]⁺.

### Compound 30

### 6-((((S)-1-(6-aminopyridin-3-yl)piperidin-3-yl)((2-methoxypyridin-4-yl)methyl)amino)methyl)-9-fluoro-3-methyl-10-(2-oxa-6-azaspiro[3.3]heptan-6-yl)-2H-[1,4]oxazino[2,3,4-ij]quinolin-7(3H)-one was Prepared According to the Procedure Described Herein for Step 1 in Scheme 7

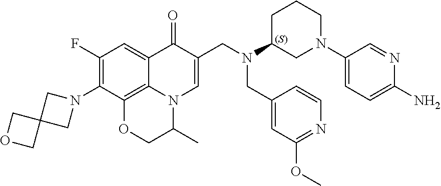

11-[[[(3S)-1-(6-amino-3-pyridyl)-3-piperidyl]-[(2-methoxy-4-pyridyl)methyl]amino]methyl]-7-fluoro-2-methyl-6-(2-oxa-6-azaspiro[3.3]heptan-6-yl)-4-oxa-1-azatricyclo[7.3.1.05,13]trideca-5(13),6,8,11-tetraen-10-one (17.2 mg, 26.80 umol, 21.54% yield, 100% purity) was obtained as white solid. ¹H NMR (DMSO-d₆, 400 MHz) δ 7.99 (d, J=4.8 Hz, 1H), 7.86 (d, J=7.6 Hz, 1H), 7.59 (d, J=1.6 Hz, 1H), 7.27 (dd, J=2.4 Hz, 16.0 Hz, 1H), 7.14-7.12 (m, 1H), 6.99 (d, J=1.6 Hz, 1H), 6.81 (s, 1H), 6.37 (dd, J=3.6 Hz, 8.8 Hz, 1H), 5.37-5.34 (m, 2H), 4.69 (s, 4H), 4.52˜4.50 (m, 1H), 4.40 (s, 4H), 4.35 (d, J=11.6 Hz, 1H), 4.14 (d, J=10.8 Hz, 1H), 3.77 (s, 3H), 3.74˜3.69 (m, 2H), 3.62˜3.57 (m, 1H), 3.54˜3.51 (m, 1H), 3.49˜3.46 (m, 1H), 3.24˜3.20 (m, 1H), 2.76-2.71 (m, 1H), 2.42˜2.37 (m, 2H), 1.96-1.92 (m, 1H), 1.77-1.73 (m, 1H), 1.50-1.37 (m, 2H), 1.29 (dd, J=4.8 Hz, 6.4 Hz, 3H). LCMS: 100.00% (220 nm), 100.00% (254 nm). MS (ESI): mass calcd. For C₃₅H₄₀FN₇O₄ 641.31 m/z found 642.5 [M+H]⁺.

### Compound 31

### 6-((((S)-1-(6-aminopyridin-3-yl)piperidin-3-yl)((2-methoxypyridin-4-yl)methyl)amino)methyl)-9-fluoro-10-(4-hydroxypiperidin-1-yl)-3-methyl-2H-[1,4]oxazino[2,3,4-ij]quinolin-7(3H)-one was Prepared According to the Procedure Described Herein for Step 1 in Scheme 7

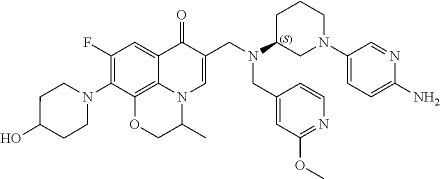

11-[[[(3S)-1-(6-amino-3-pyridyl)-3-piperidyl]-[(2-methoxy-4-pyridyl)methyl]amino]methyl]-7-fluoro-6-(4-hydroxy-1-piperidyl)-2-methyl-4-oxa-1-azatricyclo[7.3.1.05,13]trideca-5(13),6,8,11-tetraen-10-one (9.2 mg, 13.77 umol, 11.07% yield, 96.34% purity) was obtained as off white solid. ¹H NMR (DMSO-d₆, 400 MHz) δ 7.99 (d, J=5.2 Hz, 1H), 7.95 (d, J=8.0 Hz, 1H), 7.60 (d, J=2.4 Hz, 1H), 7.32 (dd, J=2.4 Hz, 12.4 Hz, 1H), 7.14 (dd, J=2.8 Hz, 9.2 Hz, 1H), 6.99 (d, J=4.8 Hz, 1H), 6.81 (s, 1H), 6.37 (dd, J=3.6 Hz, 9.2 Hz, 1H), 5.37-5.34 (m, 2H), 4.65 (d, J=4.4 Hz, 1H), 4.58˜4.56 (m, 1H), 4.44 (d, J=10.4 Hz, 1H), 4.25 (d, J=9.2 Hz, 1H), 3.76 (s, 3H), 3.71˜3.70 (m, 1H), 3.66˜3.62 (m, 1H), 3.61˜3.60 (m, 1H), 3.57˜3.54 (m, 1H), 3.50˜3.47 (m, 1H), 3.30-3.27 (m, 1H), 3.24-3.21 (m, 1H), 3.09-3.03 (m, 2H), 2.77˜2.71 (m, 2H), 2.60˜2.57 (m, 2H), 2.43˜2.32 (m, 1H), 1.97˜1.94 (m, 1H), 1.82˜1.73 (m, 3H), 1.53˜1.41 (m, 4H), 1.31 (dd, J=4.8 Hz, 6.4 Hz, 3H). LCMS: 96.34% (220 nm), 96.65% (254 nm). MS (ESI): mass calcd. For C₃₉H₄₂FN₇O₃ 643.33 m/z found 644.5 [M+H]⁺.

### Compound 32

### 6-((((S)-1-(6-aminopyridin-3-yl)piperidin-3-yl)((2-methoxypyridin-4-yl)methyl)amino)methyl)-10-(3,4-dimethylpiperazin-1-yl)-9-fluoro-3-methyl-2H-[1,4]oxazino[2,3,4-ij]quinolin-7(3H)-one was Prepared According to the Procedure Described Herein for Step 1 in Scheme 7

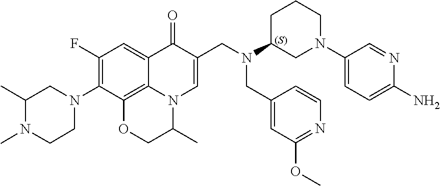

11-[[[(3S)-1-(6-amino-3-pyridyl)-3-piperidyl]-[(2-methoxy-4-pyridyl)methyl]amino]methyl]-6-(3,4-dimethylpiperazin-1-yl)-7-fluoro-2-methyl-4-oxa-1-azatricyclo[7.3.1.0^{5,13}]trideca-5(13),6,8,11-tetraen-10-one (10.2 mg, 15.53 umol, 12.48% yield, 100% purity) was obtained as white solid. ¹H NMR (DMSO-d₆, 400 MHz) δ 7.99 (d, J=5.6 Hz, 1H), 7.95 (d, J=8.0 Hz, 1H), 7.60 (d, J=2.8 Hz, 1H), 7.33 (dd, J=12.8 Hz, 2.4 Hz, 1H), 7.15-7.12 (m, 1H), 6.99 (d, J=4.8 Hz, 1H), 6.81 (s, 1H), 6.37 (dd, J=8.8 Hz, 3.6 Hz, 1H), 5.36 (d, J=3.6 Hz, 2H), 4.58-4.56 (m, 1H), 4.44 (d, J=11.2 Hz, 1H), 4.26 (d, J=9.6 Hz, 1H), 3.76 (s, 3H), 3.71˜3.70 (m, 1H), 3.66-3.47 (m, 3H), 3.27-3.20 (m, 2H), 3.16-3.09 (m, 2H), 2.92˜2.85 (m, 1H), 2.77-2.71 (m, 2H), 2.60-2.57 (m, 1H), 2.44-2.37 (m, 2H), 2.30-2.23 (m, 1H), 2.21 (s, 3H), 2.17-2.11 (m, 1H), 1.97-1.94 (m, 1H), 1.76-1.73 (m, 1H), 1.53-1.37 (m, 2H), 1.31 (dd, J=6.4 Hz, 4.4 Hz, 3H), 0.98 (d, J=6.0 Hz, 3H). LCMS: 100% (220 nm), 100% (254 nm). MS (ESI): mass calcd. For C₃₆H₄₅FN₈O₃ 656.36 m/z found 657.2 [M+H]⁺.

### Compound 33

### 6-((((S)-1-(6-aminopyridin-3-yl)piperidin-3-yl)((2-methoxypyridin-4-yl)methyl)amino)methyl)-10-(3,4-dihydroisoquinolin-2(1H)-yl)-9-fluoro-3-methyl-2H-[1,4]oxazino[2,3,4-ij]quinolin-7(3H)-one was Prepared According to the Procedure Described Herein for Step 1 in Scheme 7

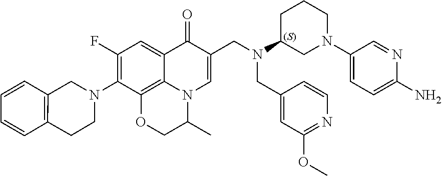

11-[[[(3S)-1-(6-amino-3-pyridyl)-3-piperidyl]-[(2-methoxy-4-pyridyl)methyl]amino]methyl]-6-(3,4-dihydro-1H-isoquinolin-2-yl)-7-fluoro-2-methyl-4-oxa-1-azatricyclo[7.3.1.05,13]trideca-5(13),6,8,11-tetraen-10-one (23.1 mg, 34.10 umol, 19.18% yield, 99.76% purity) was obtained as off white solid. ¹H NMR (DMSO-d₆, 400 MHz) δ 7.99 (t, J=5.6 Hz, 2H), 7.61 (d, J=2.4 Hz, 1H), 7.38 (dd, J=2.4 Hz, 12.4 Hz, 1H), 7.16-7.12 (m, 4H), 7.10-7.08 (m, 1H), 7.01˜7.00 (m, 1H), 6.82 (s, 1H), 6.37 (dd, J=3.6 Hz, 8.8 Hz, 1H), 5.37 (d, J=3.2 Hz, 2H), 4.61˜4.59 (m, 1H), 4.45 (d, J=11.6 Hz, 1H), 4.37 (s, 2H), 4.29 (d, J=9.6 Hz, 1H), 3.77 (s, 3H), 3.72 (d, J=4.8 Hz, 1H), 3.67˜3.61 (m, 1H), 3.59˜3.55 (m, 1H), 3.51˜3.47 (m, 3H), 3.25˜3.21 (m, 1H), 2.91 (t, J=6.0 Hz, 2H), 2.78-2.71 (m, 1H), 2.56-2.54 (m, 1H), 2.41˜2.38 (m, 2H), 2.00-1.93 (m, 1H), 1.77-1.75 (m, 1H), 1.50-1.41 (m, 2H), 1.33 (dd, J=4.4 Hz, 6.0 Hz, 3H). LCMS: 99.76% (220 nm), 99.74% (254 nm). MS (ESI): mass calcd. For C₃₉H₄₂FN₇O₃ 675.33 m/z found 676.5 [M+H]⁺.

### Compound 34

### 6-((((S)-1-(6-aminopyridin-3-yl)piperidin-3-yl)((2-methoxypyridin-4-yl)methyl)amino)methyl)-10-(3-aminopyrrolidin-1-yl)-9-fluoro-3-methyl-2H-[1,4]oxazino[2,3,4-ij]quinolin-7(3H)-one Hydrochloride was Prepared According to the Procedure Described Herein for Step 1 in Scheme 7

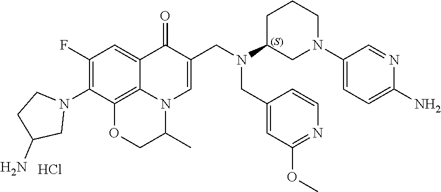

11-[[[(3S)-1-(6-amino-3-pyridyl)-3-piperidyl]-[(2-methoxy-4-pyridyl)methyl]amino]methyl]-6-(3-aminopyrrolidin-1-yl)-7-fluoro-2-methyl-4-oxa-1-azatricyclo[7.3.1.0^{5,13}]trideca-5(13),6,8,11-tetraen-10-one (4.4 mg, 6.61 umol, 9.64% yield, 100% purity, HCl) was obtained as yellow solid. ¹H NMR (DMSO-d₆, 400 MHz) δ 14.15-14.00 (m, 1H), 8.45-8.39 (m, 4H), 8.09 (s, 1H), 7.97-7.95 (m, 1H), 7.67-7.62 (m, 1H), 7.75-7.52 (m, 1H), 7.39-7.35 (m, 2H), 7.18-7.15 (m, 1H), 7.02˜7.00 (m, 1H), 4.47˜4.20 (m, 7H), 3.88-3.86 (m, 2H), 3.81 (s, 3H), 3.79-3.77 (m, 2H), 3.21˜3.14 (m, 3H), 3.07-3.02 (m, 1H), 2.69-2.58 (m, 3H), 2.12˜2.06 (m, 1H), 2.04-1.97 (m, 1H), 1.94˜1.91 (m, 2H), 1.57˜1.52 (m, 1H), 1.45˜1.43 (m, 3H). HPLC: 100% (220 nm), 100% (254 nm). MS (ESI): mass calcd. For C₃₄H₄₁FN₈O₃ 628.33 m/z found 629.4 [M+H]⁺.

### Compound 35

### 6-((((S)-1-(6-aminopyridin-3-yl)piperidin-3-yl)((2-methoxypyridin-4-yl)methyl)amino)methyl)-10-((3S,5R)-3,5-dimethylpiperazin-1-yl)-9-fluoro-3-methyl-2H-[1,4]oxazino[2,3,4-ij]quinolin-7(3H)-one was Prepared According to the Procedure Described Herein for Step 1 in Scheme 7

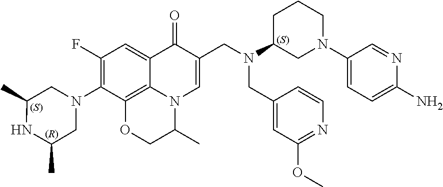

11-[[[(3S)-1-(6-amino-3-pyridyl)-3-piperidyl]-[(2-methoxy-4-pyridyl)methyl]amino]methyl]-6-[(3S,5R)-3,5-dimethylpiperazin-1-yl]-7-fluoro-2-methyl-4-oxa-1-azatricyclo[7.3.1.05,13]trideca-5(13),6,8,11-tetraen-10-one (17 mg, 25.88 umol, 14.56% yield) was obtained as pale yellow solid. ¹H NMR (DMSO-d₆, 400 MHz) δ 8.00 (d, J=5.6 Hz, 1H), 7.96 (d, J=8.0 Hz, 1H), 7.61 (d, J=2.8 Hz, 1H), 7.34 (dd, J=2.4 Hz, 12.8 Hz, 1H), 7.15-7.13 (m, 1H), 7.00 (d, J=4.8 Hz, 1H), 6.81 (s, 1H), 6.38 (dd, J=3.6 Hz, 8.8 Hz, 1H), 5.37 (d, J=3.2 Hz, 2H), 4.57˜4.56 (m, 1H), 4.45 (d, J=10.4 Hz, 1H), 4.25˜4.23 (m, 1H), 3.76 (s, 3H), 3.75 (s, 1H), 3.70 (d, J=4.4 Hz, 1H), 3.61˜3.60 (m, 1H), 3.59˜3.57 (m, 2H), 3.47 (s, 1H), 3.26˜3.18 (m, 1H), 3.06 (d, J=10.8 Hz, 2H), 2.86-2.84 (m, 2H), 2.76-2.69 (m, 1H), 2.66 (d, J=7.2 Hz, 1H), 2.57 (t, J=11.6 Hz, 1H), 2.40-2.36 (m, 1H), 2.01˜1.94 (m, 2H), 1.80-1.74 (m, 1H), 1.43-1.36 (m, 2H), 1.31 (dd, J=4.4 Hz, 6.4 Hz, 3H), 0.95 (d, J=6.0 Hz, 6H). MS: 99.16% (220 nm), 100% (254 nm). MS (ESI): mass calcd. For C₃₆H₄₅FN₈O₃ 656.36 m/z found 657.5 [M+H]⁺.

### Compound 36

### 6-((((S)-1-(6-aminopyridin-3-yl)piperidin-3-yl)((2-methoxypyridin-4-yl)methyl)amino)methyl)-9-fluoro-3-methyl-10-(4-morpholinopiperidin-1-yl)-2H-[1,4]oxazino[2,3,4-ij]quinolin-7(3H)-one was Prepared According to the Procedure Described Herein for Step 1 in Scheme 7

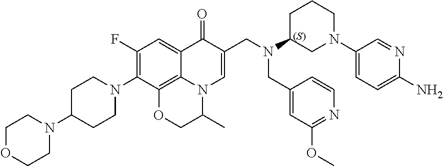

11-[[[(3S)-1-(6-amino-3-pyridyl)-3-piperidyl]-[(2-methoxy-4-pyridyl)methyl]amino]methyl]-7-fluoro-2-methyl-6-(4-morpholino-1-piperidyl)-4-oxa-1-azatricyclo[7.3.1.05,13]trideca-5(13),6,8,11-tetraen-10-one (12.7 mg, 17.79 umol, 14.29% yield, 99.83% purity) was obtained as off white solid. ¹H NMR (DMSO-d₆, 400 MHz) δ 7.99 (d, J=5.2 Hz, 1H), 7.96 (d, J=8.0 Hz, 1H), 7.60 (d, J=2.0 Hz, 1H), 7.33 (dd, J=2.0 Hz, 14.4 Hz, 1H), 7.14 (dt, J=8.8 Hz, 2.8 Hz, 1H), 6.99 (d, J=4.4 Hz, 1H), 6.81 (s, 1H), 6.37 (dd, J=3.6 Hz, 8.8 Hz, 1H), 5.36 (d, J=3.2 Hz, 2H), 4.60˜4.54 (m, 1H), 4.44 (d, J=10.8 Hz, 1H), 4.25 (d, J=9.6 Hz, 1H), 3.76 (s, 3H), 3.70 (d, J=4.4 Hz, 1H), 3.66˜3.63 (m, 1H), 3.59˜3.58 (m, 4H), 3.50˜3.47 (m, 1H), 3.25˜3.21 (m, 1H), 3.13˜3.06 (m, 2H), 2.74-2.70 (m, 3H), 2.60-2.57 (m, 4H), 2.43˜2.37 (m, 3H), 2.33˜2.28 (m, 2H), 1.97-1.94 (m, 1H), 1.82˜1.74 (m, 3H), 1.54-1.41 (m, 4H), 1.31 (d, J=3.2 Hz, 3H). LCMS: 99.83% (220 nm), 100.00% (254 nm). MS (ESI): mass calcd. For C₃₉H₄₉FN₈O₄ 712.39 m/z found 713.5 [M+H]⁺.

### Compound 37

### 6-((((S)-1-(6-aminopyridin-3-yl)piperidin-3-yl)((2-methoxypyridin-4-yl)methyl)amino)methyl)-9-fluoro-3-methyl-10-(2-methylpiperidin-1-yl)-2H-[1,4]oxazino[2,3,4-ij]quinolin-7(3H)-one was Prepared According to the Procedure Described Herein for Step 1 in Scheme 7

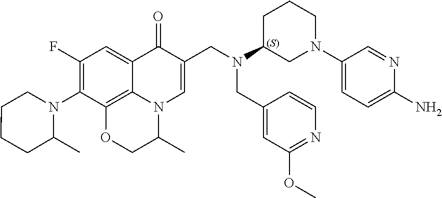

11-[[[(3S)-1-(6-amino-3-pyridyl)-3-piperidyl]-[(2-methoxy-4-pyridyl)methyl]amino]methyl]-7-fluoro-2-methyl-6-(2-methyl-1-piperidyl)-4-oxa-1-azatricyclo[7.3.1.0¹³]trideca-5(13),6,8,11-tetraen-10-one (10.7 mg, 16.10 umol, 6.04% yield, 96.573% purity) was obtained as pale pink solid. ¹H NMR (DMSO-d₆, 400 MHz) δ 7.99 (d, J=5.2 Hz, 2H), 7.61 (d, J=2.4 Hz, 1H), 7.33 (dd, J=1.2 Hz, 11.2 Hz, 1H), 7.16-7.12 (m, 1H), 6.99 (d, J=4.4 Hz, 1H), 6.81 (s, 1H), 6.37 (dd, J=3.2 Hz, 9.2 Hz, 1H), 5.36 (d, J=3.2 Hz, 2H), 4.62˜4.59 (m, 1H), 4.47 (d, J=11.6 Hz, 1H), 4.25 (d, J=11.6 Hz, 1H), 3.76 (s, 3H), 3.71˜3.70 (m, 1H), 3.66˜3.55 (m, 3H), 3.51˜3.48 (m, 1H), 3.37˜3.34 (m, 1H), 3.25˜3.21 (m, 1H), 3.06˜2.98 (m, 2H), 2.78˜2.72 (m, 1H), 2.61˜2.58 (m, 1H), 2.41˜2.38 (m, 1H), 1.98˜1.95 (m, 1H), 1.76˜1.71 (m, 3H), 1.60˜1.56 (m, 2H), 1.49-1.41 (m, 3H), 1.31˜1.29 (m, 4H), 0.85 (dd, J=2.8 Hz, 6.0 Hz, 3H). LCMS: 96.57% (220 nm), 96.67% (254 nm). MS (ESI): mass calcd. For C₃₆H₄₄FN₇O₃ 641.35 m/z found 642.5 [M+H]⁺.

### Compound 38

### 6-((((S)-1-(6-aminopyridin-3-yl)piperidin-3-yl)((2-methoxypyridin-4-yl)methyl)amino)methyl)-9-fluoro-10-(3-hydroxyazetidin-1-yl)-3-methyl-2H-[1,4]oxazino[2,3,4-ij]quinolin-7(3H)-one was Prepared According to the Procedure Described Herein for Step 1 in Scheme 7

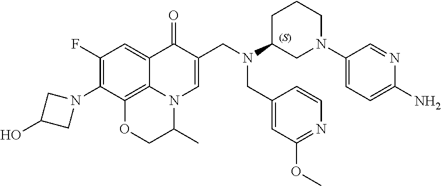

11-[[[(3S)-1-(6-amino-3-pyridyl)-3-piperidyl]-[(2-methoxy-4-pyridyl)methyl]amino]methyl]-7-fluoro-6-(3-hydroxyazetidin-1-yl)-2-methyl-4-oxa-1-azatricyclo[7.3.1.05,13]trideca-5(13),6,8,11-tetraen-10-one (14.9 mg, 23.74 umol, 8.67% yield, 98.08% purity) was obtained as yellow solid. ¹H NMR (DMSO-d₆, 400 MHz) δ 7.99 (d, J=5.2 Hz, 1H), 7.86 (d, J=7.6 Hz, 1H), 7.60 (d, J=2.8 Hz, 1H), 7.27 (dd, J=2.0 Hz, 13.6 Hz, 1H), 7.14 (dt, J=2.4 Hz, 8.4 Hz, 1H), 6.99 (d, J=5.2 Hz, 1H), 6.81 (s, 1H), 6.37 (dd, J=3.2 Hz, 8.8 Hz, 1H), 5.36 (d, J=2.8 Hz, 2H), 4.52˜4.50 (m, 1H), 4.45 (s, 3H), 4.34 (d, J=10.8 Hz, 1H), 4.14 (d, J=9.6 Hz, 1H), 3.95-3.94 (m, 2H), 3.77 (s, 3H), 3.70˜3.69 (m, 1H), 3.65˜3.61 (m, 1H), 3.58˜3.57 (m, 1H), 3.55 (s, 1H), 3.51˜3.47 (m, 2H), 3.24˜3.21 (m, 1H), 2.54 (s, 1H), 2.43˜2.37 (m, 2H), 1.96˜1.94 (m, 1H), 1.77-1.73 (m, 1H), 1.50-1.40 (m, 2H), 1.29 (dd, J=4.8 Hz, 6.0 Hz, 3H). LCMS: 98.08% (220 nm), 99.10% (254 nm). MS (ESI): mass calcd. For C₃₃H₃₈FN₇O₄ 615.30 m/z found 616.5 [M+H]⁺.

### Compound 39

### 6-((((S)-1-(6-aminopyridin-3-yl)piperidin-3-yl)((2-methoxypyridin-4-yl)methyl)amino)methyl)-9-fluoro-3-methyl-10-(piperazin-1-yl)-2H-[1,4]oxazino[2,3,4-ij]quinolin-7(3H)-one was Prepared According to the Procedure Described Herein for Step 1 in Scheme 7

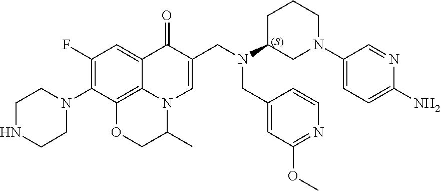

11-[[[(3S)-1-(6-amino-3-pyridyl)-3-piperidyl]-[(2-methoxy-4-pyridyl)methyl]amino]methyl]-7-fluoro-2-methyl-6-piperazin-1-yl-4-oxa-1-azatricyclo[7.3.1.0^{5,13}]trideca-5(13),6,8,11-tetraen-10-one (33 mg, 52.16 umol, 29.35% yield, 99.38% purity) was obtained as white solid. ¹H NMR (DMSO-d₆, 400 MHz) δ 8.00-7.95 (m, 2H), 7.60 (d, J=2.4 Hz, 1H), 7.34 (dd, J=2.4 Hz, 12.8 Hz, 1H), 7.15-7.12 (m, 1H), 6.99 (d, J=4.8 Hz, 1H), 6.81 (s, 1H), 6.37 (dd, J=3.6 Hz, 8.8 Hz, 1H), 5.37 (d, J=2.8 Hz, 2H), 4.58-4.57 (m, 1H), 4.44 (d, J=10.4 Hz, 1H), 4.26 (d, J=9.6 Hz, 1H), 3.76 (s, 3H), 3.71˜3.70 (m, 1H), 3.66˜3.54 (m, 2H), 3.50˜3.47 (m, 1H), 3.35˜3.30 (m, 1H), 3.25-3.21 (m, 1H), 3.17-3.13 (m, 4H), 2.84-2.82 (m, 4H), 2.77-2.71 (m, 1H), 2.60-2.55 (m, 1H), 2.43˜2.37 (m, 1H), 1.97-1.94 (m, 1H), 1.77-1.73 (m, 1H), 1.49-1.37 (m, 2H), 1.31 (dd, J=4.8 Hz, 6.0 Hz, 3H). LCMS: 99.38% (220 nm), 99.55% (254 nm). MS (ESI): mass calcd. For C₃₄H₄₁FN₈O₃ 628.33 m/z found 629.5 [M+H]⁺.

### Compound 40

### 6-((((S)-1-(6-aminopyridin-3-yl)piperidin-3-yl)((2-methoxypyridin-4-yl)methyl)amino)methyl)-9-fluoro-3-methyl-10-(3-methylpiperazin-1-yl)-2H-[1,4]oxazino[2,3,4-ij]quinolin-7(3H)-one was Prepared According to the Procedure Described Herein for Step 1 in Scheme 7

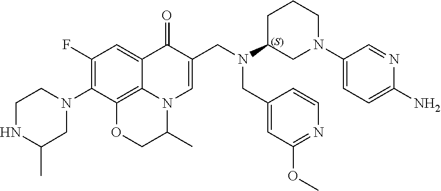

11-[[[(3S)-1-(6-amino-3-pyridyl)-3-piperidyl]-[(2-methoxy-4-pyridyl)methyl]amino]methyl]-7-fluoro-2-methyl-6-(3-methylpiperazin-1-yl)-4-oxa-1-azatricyclo[7.3.1.05,13]trideca-5(13),6,8,11-tetraen-10-one (48 mg, 74.40 umol, 13.95% yield, 99.63% purity) was obtained as white solid. ¹H NMR (DMSO-d₆, 400 MHz) δ 8.00-7.95 (m, 2H), 7.60 (d, J=2.8 Hz, 1H), 7.33 (dd, J=2.4 Hz, 12.8 Hz, 1H), 7.14 (dt, J=2.4 Hz, 9.2 Hz, 1H), 6.99 (d, J=5.2 Hz, 1H), 6.81 (s, 1H), 6.37 (dd, J=3.6 Hz, 8.8 Hz, 1H), 5.37 (d, J=3.2 Hz, 2H), 4.58-4.56 (m, 1H), 4.44 (d, J=11.2 Hz, 1H), 4.25 (d, J=10.8 Hz, 1H), 3.76-3.75 (m, 3H), 3.71˜3.69 (m, 1H), 3.66-3.60 (m, 1H), 3.57˜3.54 (m, 1H), 3.50˜3.47 (m, 1H), 3.24˜3.21 (m, 1H), 3.09˜3.07 (m, 3H), 2.86˜2.67 (m, 5H), 2.60-2.54 (m, 1H), 2.43˜2.37 (m, 2H), 2.17-1.95 (m, 1H), 1.97-1.95 (m, 1H), 1.77-1.73 (m, 1H), 1.49-1.37 (m, 2H), 1.31 (dd, J=5.2 Hz, 6.4 Hz, 3H), 0.94 (d, J=5.6 Hz, 3H). LCMS: 99.63% (220 nm), 100% (254 nm). MS (ESI): mass calcd. For C₃₅H₄₃FN₈O₃ 642.34 m/z found 643.5 [M+H]⁺.

### Compound 41

### 6-((((S)-1-(6-aminopyridin-3-yl)piperidin-3-yl)((2-methoxypyridin-4-yl)methyl)amino)methyl)-9-fluoro-3-methyl-10-(2-methylpiperazin-1-yl)-2H-[1,4]oxazino[2,3,4-ij]quinolin-7(3H)-one was Prepared According to the Procedure Described Herein for Step 1 in Scheme 7

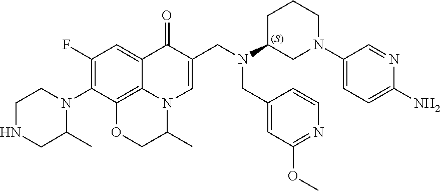

11-[[[(3S)-1-(6-amino-3-pyridyl)-3-piperidyl]-[(2-methoxy-4-pyridyl)methyl]amino]methyl]-7-fluoro-2-methyl-6-(2-methylpiperazin-1-yl)-4-oxa-1-azatricyclo[7.3.1.05,13]trideca-5(13),6,8,11-tetraen-10-one (1.3 mg, 2.02 umol, 3.79e-1% yield, 99.11% purity) was obtained as off white solid. ¹H NMR (DMSO-d₆, 400 MHz) δ 8.00-7.97 (m, 2H), 7.60 (d, J=2.8 Hz, 1H), 7.21 (dd, J=2.0 Hz, 8.4 Hz, 1H), 7.14 (d, J=7.6 Hz, 1H), 7.00 (d, J=4.8 Hz, 1H), 6.82 (s, 1H), 6.37 (dd, J=3.6 Hz, 9.2 Hz, 1H), 5.36 (d, J=3.2 Hz, 2H), 4.63˜4.60 (m, 1H), 4.47 (d, J=10.8 Hz, 1H), 4.31 (d, J=10.0 Hz, 1H), 3.80-3.77 (m, 4H), 3.71˜3.70 (m, 1H), 3.66-3.61 (m, 1H), 3.58˜3.54 (m, 1H), 3.50˜3.48 (m, 1H), 3.24˜3.17 (m, 4H), 2.95˜2.92 (m, 1H), 2.88˜2.83 (m, 2H), 2.77-2.72 (m, 1H), 2.61˜2.55 (m, 2H), 2.43˜2.38 (m, 1H), 2.31˜2.24 (m, 1H), 1.98-1.95 (m, 1H), 1.77-1.74 (m, 1H), 1.50-1.38 (m, 2H), 1.32 (dd, J=4.8 Hz, 6.4 Hz, 3H), 1.01 (dd, J=3.2 Hz, 6.0 Hz, 3H). LCMS: 99.11% (220 nm), 99.72% (254 nm). MS (ESI): mass calcd. For C₃₅H₄₃FN₈O₃ 642.34 m/z found 643.5 [M+H]⁺.

### Compound 42

### 6-((((S)-1-(6-aminopyridin-3-yl)piperidin-3-yl)((2-methoxypyridin-4-yl)methyl)amino)methyl)-9-fluoro-3-methyl-10-(3-(methylamino)piperidin-1-yl)-2H-[1,4]oxazino[2,3,4-ij]quinolin-7(3H)-one Hydrochloride was Prepared According to the Procedure Described Herein for Step 1 in Scheme 7

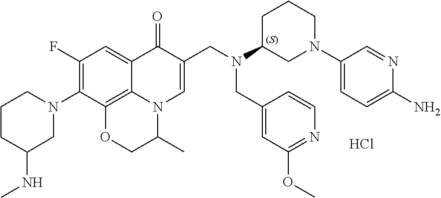

11-[[[(3S)-1-(6-amino-3-pyridyl)-3-piperidyl]-[(2-methoxy-4-pyridyl)methyl]amino]methyl]-7-fluoro-2-methyl-6-[3-(methylamino)-1-piperidyl]-4-oxa-1-azatricyclo[7.3.1.0^{5,13}]trideca-5(13),6,8,11-tetraen-10-one (24.3 mg, 33.85 umol, 9.52% yield, 96.56% purity, HCl) was obtained as pale yellow solid. ¹H NMR (DMSO-d₆, 400 MHz) δ 14.19-14.10 (m, 1H), 9.31˜9.20 (m, 2H), 8.52˜8.48 (m, 1H), 8.11 (t, J=4.8 Hz, 1H), 7.99˜7.95 (m, 1H), 7.55˜7.52 (m, 1H), 7.42˜7.37 (m, 2H), 7.20 (d, J=6.8 Hz, 1H), 7.00 (dd, J=7.2 Hz, 9.6 Hz, 1H), 4.49˜4.44 (m, 3H), 4.32˜4.21 (m, 4H), 3.77 (d, J=2.8 Hz, 3H), 3.59˜3.55 (m, 1H), 3.41˜3.34 (m, 2H), 3.24˜3.11 (m, 4H), 3.08˜3.06 (m, 2H), 2.63˜2.54 (m, 5H), 2.13˜2.09 (m, 1H), 1.90˜1.83 (m, 3H), 1.67-1.53 (m, 3H), 1.46-1.44 (m, 3H). LCMS: 96.56% (220 nm), 96.68% (254 nm). MS (ESI): mass calcd. For C₃₆H₄₅FN₈O₃ 656.36 m/z found 657.5 [M+H]⁺.

### Compound 43

### 6-((((S)-1-(6-aminopyridin-3-yl)piperidin-3-yl)((2-methoxypyridin-4-yl)methyl)amino)methyl)-9-fluoro-3-methyl-10-(methyl(piperidin-3-yl)amino)-2H-[1,4]oxazino[2,3,4-ij]quinolin-7(3H)-one Hydrochloride was Prepared According to the Procedure Described Herein for Step 1 in Scheme 7

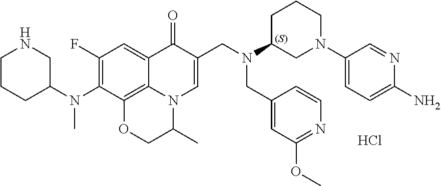

11-[[[(3S)-1-(6-amino-3-pyridyl)-3-piperidyl]-[(2-methoxy-4-pyridyl)methyl]amino]methyl]-7-fluoro-2-methyl-6-[methyl(3-piperidyl)amino]-4-oxa-1-azatricyclo[7.3.1.0^{5,13}]trideca-5(13),6,8,11-tetraen-10-one (3.6 mg, 5.18 umol, 1.46% yield, 99.75% purity, HCl) was obtained as pale yellow solid. ¹H NMR (DMSO-d₆, 400 MHz) δ 10.19-10.11 (m, 1H), 9.75-9.71 (m, 1H), 9.22˜9.07 (m, 2H), 8.46 (s, 1H), 8.22 (d, J=5.2 Hz, 1H), 7.96 (dd, J=2.4 Hz, 10.0 Hz, 1H), 7.48-7.45 (m, 2H), 7.30 (d, J=5.2 Hz, 1H), 7.18-7.14 (m, 2H), 4.63˜4.61 (m, 1H), 4.54˜4.49 (m, 1H), 4.40 (s, 2H), 4.37˜4.32 (m, 1H), 4.27˜4.24 (m, 2H), 3.87 (s, 3H), 3.84˜3.83 (m, 1H), 3.58˜3.53 (m, 2H), 3.27˜3.19 (m, 4H), 3.08˜3.03 (m, 2H), 2.75˜2.67 (m, 1H), 2.58˜2.55 (m, 3H), 2.11˜2.07 (m, 2H), 1.92˜1.81 (m, 3H), 1.67-1.56 (m, 3H), 1.43 (d, J=6.4 Hz, 3H). LCMS: 99.75% (220 nm), 99.80% (254 nm). MS (ESI): mass calcd. For C₃₆H₄₅FN₈O₃ 656.36 m/z found 657.5 [M+H]⁺.

### Compound 44

### 6-((((S)-1-(6-aminopyridin-3-yl)piperidin-3-yl)((2-methoxypyridin-4-yl)methyl)amino)methyl)-9-fluoro-10-(3-hydroxy-1-oxa-8-azaspiro[4.5]decan-8-yl)-3-methyl-2H-[1,4]oxazino[2,3,4-ij]quinolin-7(3H)-one was Prepared According to the Procedure Described Herein for Step 1 in Scheme 7

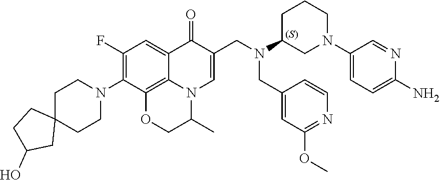

11-[[[(3S)-1-(6-amino-3-pyridyl)-3-piperidyl]-[(2-methoxy-4-pyridyl) methyl]amino]methyl]-7-fluoro-6-(3-hydroxy-1-oxa-8-azaspiro[4.5]decan-8-yl)-2-methyl-4-oxa-1-azatricyclo [7.3.1.05,13]trideca-5(13),6,8,11-tetraen-10-one (35.4 mg, 47.89 umol, 12.55% yield, 94.69% purity) was obtained as yellow solid. ¹H NMR (DMSO-d₆, 400 MHz) δ 8.00-7.94 (m, 2H), 7.60 (d, J=2.8 Hz, 1H), 7.33 (dd, J=2.4 Hz, 12.4 Hz, 1H), 7.14 (dt, J=2.8 Hz, 8.8 Hz, 1H), 6.99 (d, J=4.8 Hz, 1H), 6.81 (s, 1H), 6.37 (dd, J=3.6 Hz, 8.8 Hz, 1H), 5.36 (d, J=3.6 Hz, 2H), 4.86 (d, J=3.6 Hz, 1H), 4.57 (dd, J=2.0 Hz, 6.8 Hz, 1H), 4.44 (d, J=10.0 Hz, 1H), 4.35˜4.33 (m, 1H), 4.25 (d, J=9.2 Hz, 1H), 3.82 (dd, J=4.8 Hz, 9.2 Hz, 1H), 3.76 (s, 4H), 3.71˜3.63 (m, 2H), 3.61˜3.54 (m, 3H), 3.50˜3.47 (m, 1H), 3.23˜3.21 (m, 1H), 3.05 (dd, J=5.6 Hz, 11.6 Hz, 2H), 2.77-2.71 (m, 1H), 2.60-2.54 (m, 1H), 2.43˜2.37 (m, 1H), 1.97-1.88 (m, 2H), 1.81˜1.72 (m, 4H), 1.63-1.61 (m, 2H), 1.49-1.38 (m, 2H), 1.31 (dd, J=4.8 Hz, 6.4 Hz, 3H). LCMS: 94.69% (220 nm), 98.36% (254 nm). MS (ESI): mass calcd. For C₃₈H₄₆FN₇O₅ 699.35 m/z found 700.5 [M+H]⁺.

### Compound 45

### 6-((((S)-1-(6-aminopyridin-3-yl)piperidin-3-yl)((2-methoxypyridin-4-yl)methyl)amino)methyl)-9-fluoro-3-methyl-10-(7-methyl-2,7-diazaspiro[3.5]nonan-2-yl)-2H-[1,4]oxazino[2,3,4-ij]quinolin-7(3H)-one was Prepared According to the Procedure Described Herein for Step 1 in Scheme 7

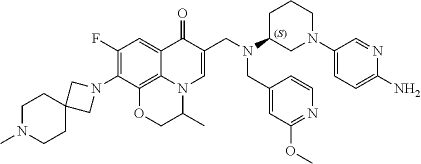

11-[[[(3S)-1-(6-amino-3-pyridyl)-3-piperidyl]-[(2-methoxy-4-pyridyl)methyl]amino]methyl]-7-fluoro-2-methyl-6-(7-methyl-2,7-diazaspiro[3.5]nonan-2-yl)-4-oxa-1-azatricyclo[7.3.1.0^{5,13}]trideca-5(13),6,8,11-tetraen-10-one (20.3 mg, 29.31 umol, 16.49% yield, 98.59% purity) was obtained as white solid. ¹H NMR (DMSO-d₆, 400 MHz) δ 8.00 (d, J=5.6 Hz, 1H), 7.87 (dd, J=8.0 Hz, 12.4 Hz, 1H), 7.60 (s, 1H), 7.32˜7.25 (m, 1H), 7.15 (d, J=8.8 Hz, 1H), 7.00-6.99 (m, 1H), 6.82 (s, 1H), 6.38 (dd, J=3.2 Hz, 8.8 Hz, 1H), 5.37-5.32 (m, 2H), 4.54-4.49 (m, 1H), 4.41˜4.33 (m, 1H), 4.20-4.12 (m, 1H), 3.97 (d, J=3.2 Hz, 2H), 3.77 (d, J=1.6 Hz, 3H), 3.71˜3.66 (m, 2H), 3.60-3.47 (m, 5H), 3.24-3.21 (m, 1H), 2.75-2.74 (m, 1H), 3.30 (s, 4H), 2.41˜2.36 (m, 2H), 2.23 (s, 3H), 1.97˜1.94 (s, 1H), 1.88˜1.82 (m, 1H), 1.74 (s, 4H), 1.53˜1.40 (m, 2H), 1.31 (q, J=11.2 Hz, 3H). LCMS: 98.59% (220 nm), 99.03% (254 nm). MS (ESI): mass calcd. For C₃₈H₄₇FN₈O₃ 682.38 m/z found 683.5 [M+H]⁺.

### Compound 46

### 6-((((S)-1-(6-aminopyridin-3-yl)piperidin-3-yl)((2-methoxypyridin-4-yl)methyl)amino)methyl)-10-(5,5-difluorohexahydrocyclopenta[c]pyrrol-2(1H)-yl)-9-fluoro-3-methyl-2H-[1,4]oxazino[2,3,4-ij]quinolin-7(3H)-one was Prepared According to the Procedure Described Herein for Step 1 in Scheme 7

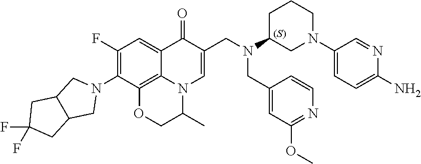

6-((((S)-1-(6-aminopyridin-3-yl)piperidin-3-yl)((2-methoxypyridin-4-yl)methyl)amino)methyl)-10-(5,5-difluorohexahydrocyclopenta[c]pyrrol-2(1H)-yl)-9-fluoro-3-methyl-2H-[1,4]oxazino[2,3,4-ij]quinolin-7(3H)-one (1.1 mg, 1.50 umol, 1.69% yield, 94.18% purity) was obtained as pale yellow solid. ¹H NMR (DMSO-d₆, 400 MHz) δ 8.00 (d, J=5.2 Hz, 1H), 7.95 (d, J=8.0 Hz, 1H), 7.61˜7.60 (m, 1H), 7.37-7.30 (m, 1H), 7.15-7.13 (m, 1H), 7.00-6.97 (m, 1H), 6.81 (s, 1H), 6.36 (dd, J=3.2 Hz, 8.8 Hz, 1H), 5.41˜5.29 (m, 2H), 4.57-4.55 (m, 1H), 4.45 (d, J=10.8 Hz, 1H), 4.24 (d, J=10.4 Hz, 1H), 3.76 (s, 3H), 3.71˜3.69 (m, 1H), 3.67-3.62 (m, 1H), 3.61˜3.57 (m, 1H), 3.54-3.46 (m, 3H), 3.24-3.21 (m, 2H), 2.78-2.72 (m, 3H), 2.41˜2.37 (m, 4H), 2.02˜1.95 (m, 4H), 1.77-1.74 (m, 1H), 1.50-1.41 (m, 3H), 1.31 (d, J=6.4 Hz, 3H). LCMS: 94.18% (220 nm), 99.76% (254 nm). MS (ESI): mass calcd. For C₃₇H₄₂F₃N₇O₃ 689.33 m/z found 690.5 [M+H]⁺.

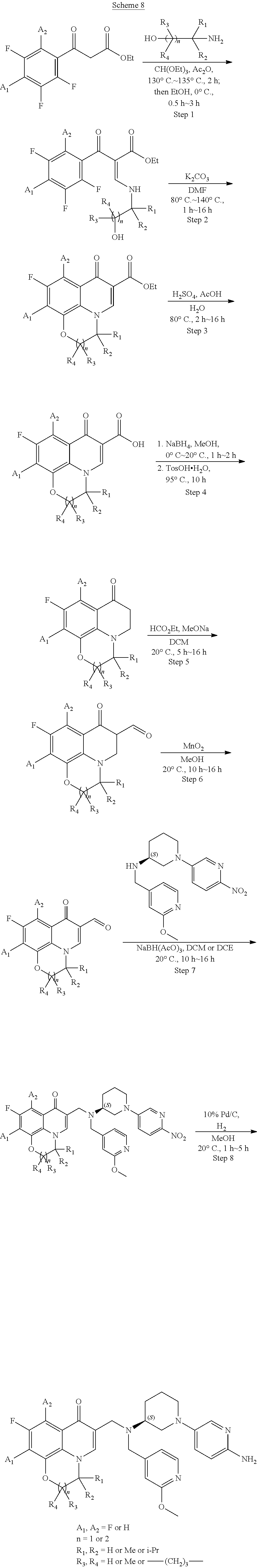

### General Procedures for Preparing Compounds in Scheme 8

### Preparation of Compounds in Scheme 8 (Step 1 in Scheme 8)

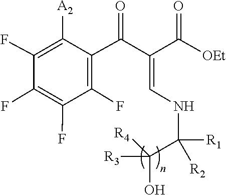

A mixture of ethyl 3-oxo-3-(2,3,4,5 or 2,3,5,6-tetrafluorophenyl)propanoate (11.36 mmol, 1 eq), Ac₂O (28.39 mmol, 2.66 mL, 2.50 eq) and triethyl orthoformate (17.03 mmol, 1.50 eq) was stirred at 130° C.˜135° C. for 2 hours. The mixture was concentrated under reduced pressure. To the above residue in EtOH (1.6 mL/mmol˜2.8 mL/mmol) was added different amine (11.24 mmol, 1 eq) dropwise very slowly at 0° C. Then the mixture was stirred at 0° C. for half an hour 3 hours. The mixture was concentrated under reduced pressure to give the desired product.

### Preparation of Compounds in Scheme 8 (Step 2 in Scheme 8)

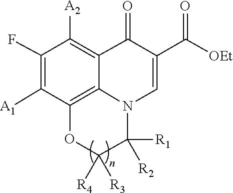

To a solution of different hydroxypropylamino (10.02 mmol, 1 eq) in DMF (3 mL/mmol˜5 mL/mmol) was added K₂CO₃ (30.06 mmol, 3 eq) at 20° C. Then the mixture was stirred at 80° C. 140° C. for 1 hour˜16 hours. The mixture was cooled to room temperature, poured into ice water at 0° C. and there was some solid formed. The solid was collected after filtration to give the desired product.

### Preparation of Compounds in Scheme 8 (Step 3 in Scheme 8)

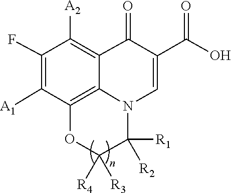

To a solution of different ester (7.76 mmol, 1 eq) in H₂O (1.1 mL/mmol˜1.3 mL/mmol) and AcOH (155.21 mmol, 20 eq) was added con. H₂SO₄ (19.40 mmol, 2.5 eq) at 20° C. Then the mixture was stirred at 80° C. for 2 hours˜16 hours. The mixture was cooled to room temperature and then poured into ice water at 0° C. and there was some solid formed. The solid was collected after filtration.

### Preparation of Compounds in Scheme 8 (Step 4 in Scheme 8)

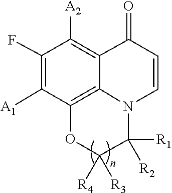

To a solution of different carboxylic acid (7.47 mmol, 1 eq) in MeOH (3 mL/mmol˜5 mL/mmol) was added NaBH₄ (33.60 mmol, 4.5 eq) in portions at 0° C. during half an hour. The mixture was stirred at 20° C. for an hour˜2 hours under N₂ atmosphere. Then TsOH·H₂O (746.77 umol, 0.1 eq) was added and the mixture was heated at 95° C. for 10 hours under N₂ atmosphere. The reaction mixture was concentrated under reduced pressure. The residue was quenched with water slowly at 0° C. and extracted with EtOAc. The combined organic layer was washed with brine, dried over Na₂SO₄, filtered and concentrated under reduced pressure. The residue was used to the next step without further purification or purified by flash silica gel chromatography (Biotage®; 12 g or 20 g or 40 g SepaFlash® Silica Flash Column, Eluent of 0˜25% or 0˜42% gradient ethyl acetate/petroleum ether @30 mL/min or 80 mL/min). The eluent was removed under reduced pressure to give the desired product.

### Preparation of Compounds in Scheme 8 (Step 5 in Scheme 8)

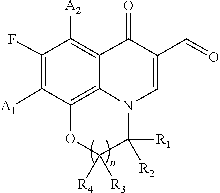

To a mixture of different ketone (4.60 mmol, 1 eq) in DCM (2.5 mL/mmol˜6.5 mL/mmol) was added NaOMe (17.93 mmol, 3.9 eq) in portions at 0° C. The mixture was stirred at 20° C. for an hour, then ethyl formate (17.93 mmol, 3.9 eq) was added dropwise at 20° C. Then the mixture was stirred at 20° C. for 5 hours˜16 hours under N₂ atmosphere. The reaction mixture was poured into ice water slowly at 0° C. and made pH=6 with 0.5N HCl. The organic layer was separated and the aqueous was extracted with DCM. The combined organic layer was washed with brine, dried over Na₂SO₄, filtered and concentrated under reduced pressure to give the desired product.

### Preparation of Compounds in Scheme 8 (Step 6 in Scheme 8)

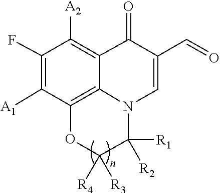

To a mixture of unsaturated carbaldehyde (4.49 mmol, 1 eq) in MeOH (3 mL/mmol˜7 mL/mmol) was added MnO₂ (22.45 mmol, 5 eq). Then the mixture was stirred at 20° C. for 10 hours˜16 hours under N₂ atmosphere. The reaction mixture was filtered through a pad of the Celite and the filtrate was concentrated under reduced pressure. The filter cake was washed with DCM. The combined filtrate was concentrated under reduced pressure to give the desired product.

### Preparation of Compounds in Scheme 8 (Step 7 in Scheme 8)

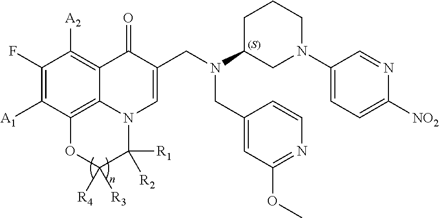

A solution of different saturated carbaldehyde (754.11 umol, 1 eq) and (3S)—N-[(2-methoxy-4-pyridyl)methyl]-1-(6-nitro-3-pyridyl)piperidin-3-amine (754.11 umol, 1 eq) in DCM (6 mL/mmol˜9 mL/mmol) or DCE (6 mL/mmol˜9 mL/mmol) was stirred at 20° C. for an hour. Then the mixture was cooled to 0° C., NaBH(OAc)₃ (980.35 umol˜1.13 mmol, 1.3 eq˜1.5 eq) was added in portions at 0° C. The mixture was stirred at 20° C. for 10 hours˜16 hours. The mixture was quenched with ice water at 0° C. and made pH=8 with sat. NaHCO₃. The mixture was extracted with DCM and i-PrOH (v:v=3:1). The combined organic layer was washed with brine, dried over Na₂SO₄, filtered and concentrated under reduced pressure to give the desired product.

### Preparation of Compounds in Scheme 8 (Step 8 in Scheme 8)

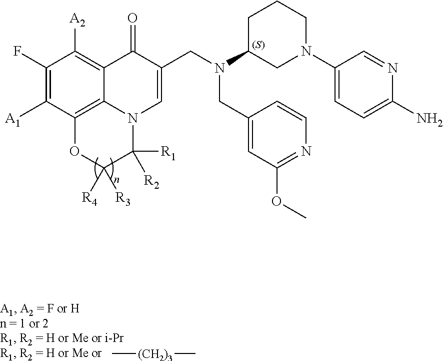

A mixture of different nitro compound (506.25 umol, 1 eq) and Pd/C (0.1 g, 10% purity) in MeOH (20 mL/mmol˜60 mL/mmol) was stirred at 20° C. for an hour˜5 hours under H₂ (15 psi). The mixture was filtered through a pad of the Celie. The filtrate was concentrated under reduced pressure. The residue was purified by prep-HPLC (column: Waters Xbridge Prep OBD C 18 150*40 mm*10 um or Waters Xbridge BEH C18 100*30 mm*10 um or Phenomenex C18 75*30 mm*3 um or Phenomenex C 18 80*40 mm*3 um or Phenomenex Luna 80*30 mm*3 um; mobile phase: [water (NH₄HCO₃)-MeCN] or [water (HCl)-MeCN]; B %: 1%-75%, 7 mins or 8 mins or 10 mins). The solvent was removed under freeze drying to give the desired product.

### Compound 47

### Preparation of (Z)-ethyl 3-((3-hydroxypropyl)amino)-2-(2,3,4,5-tetrafluorobenzoyl)acrylate (Step 1 in Scheme 8)

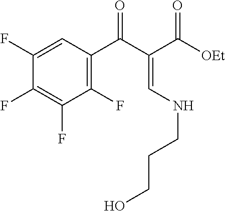 [SMILES: CCOC(=O)/C(=C\NCCCO)C(=O)c1cc(F)c(F)c(F)c1F]

A mixture of ethyl 3-oxo-3-(2,3,4,5-tetrafluorophenyl)propanoate (3 g, 11.36 mmol, 1 eq), Ac₂O (2.90 g, 28.39 mmol, 2.66 mL, 2.50 eq) and triethyl orthoformate (2.52 g, 17.03 mmol, 2.83 mL, 1.50 eq) was stirred at 135° C. for 2 hours. The mixture was concentrated under reduced pressure. To the above residue in EtOH (20 mL) was added 3-aminopropan-1-ol (844.36 mg, 11.24 mmol, 866.90 uL, 1 eq) dropwise very slowly at 0° C. Then the mixture was stirred at 0° C. for 2 hours. LCMS and TLC (petroleum ether:EtOAc=1:1, R_{f}=0.05) showed the reaction was nearly complete. The mixture was concentrated under reduced pressure. Compound ethyl (Z)-3-(3-hydroxypropylamino)-2-(2,3,4,5-tetrafluorobenzoyl)prop-2-enoate (3.8 g, 10.88 mmol, 96.78% yield) was obtained as pale yellow solid. ¹H NMR (DMSO-d₆, 400 MHz) δ 11.02 (s, 1H), 8.16-8.11 (m, 1H), 6.99-6.97 (m, 1H), 4.09 (q, J=6.4 Hz, 2H), 3.82 (t, J=6.0 Hz, 2H), 3.65 (t, J=6.4 Hz, 2H), 2.02˜1.91 (m, 2H), 1.11 (t, J=6.4 Hz, 3H).

### Preparation of ethyl 10,11-difluoro-8-oxo-2,3,4,8-tetrahydro-[1,4]oxazepino[2,3,4-ij]quinoline-7-carboxylate (Step 2 in Scheme 8)

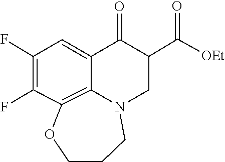 [SMILES: CCOC(=O)C1CN2CCCOc3c(F)c(F)cc(c32)C1=O]

To a solution of ethyl (E)-3-(3-hydroxypropylamino)-2-(2,3,4,5-tetrafluorobenzoyl)prop-2-enoate (3.50 g, 10.02 mmol, 1 eq) in DMF (50 mL) was added K₂CO₃ (4.15 g, 30.06 mmol, 3 eq) at 20° C. Then the mixture was stirred at 80° C. for 10 hours. LCMS and TLC (petroleum ether:EtOAc=0:1, R_{f}=0.1) showed the reaction was nearly complete. The mixture was cooled to room temperature, poured into ice water (100 mL) at 0° C. and there was some solid formed. The solid was collected after filtration. The filtrate was made pH=7 with 2N HCl at 0° C. and extracted with EtOAc (40 mL×3). The combined organic layer was washed with brine (20 mL×3), dried over Na₂SO₄, filtered and concentrated under reduced pressure. Compound ethyl 10,11-difluoro-8-oxo-2,3,4,8-tetrahydro-[1,4]oxazepino[2,3,4-ij]quinoline-7-carboxylate (2.4 g, 7.76 mmol, 77.44% yield) was obtained as pale yellow solid. ¹H NMR (DMSO-d₆, 400 MHz) δ 8.64 (s, 1H), 7.76 (dd, J=8.4 Hz, 2.0 Hz, 1H), 4.57 (t, J=6.0 Hz, 2H), 4.48 (t, J=6.8 Hz, 2H), 4.22 (q, J=7.2 Hz, 2H), 2.42˜2.38 (m, 2H), 1.28 (t, J=6.8 Hz, 3H).

### Preparation of 10,11-difluoro-8-oxo-2,3,4,8-tetrahydro-[1,4]oxazepino[2,3,4-ij]quinoline-7-carboxylic Acid (Step 3 in Scheme 8)

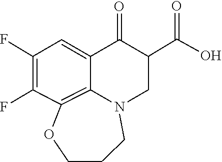 [SMILES: O=C(O)C1CN2CCCOc3c(F)c(F)cc(c32)C1=O]

To a solution of ethyl 10,11-difluoro-8-oxo-2,3,4,8-tetrahydro-[1,4]oxazepino[2,3,4-ij]quinoline-7-carboxylate (2.4, 7.76 mmol, 1 eq) in H₂O (10 mL) and AcOH (9.32 g, 155.21 mmol, 8.88 mL, 20 eq) was added con. H₂SO₄ (1.90 g, 19.40 mmol, 1.03 mL, 2.5 eq) at 20° C. Then the mixture was stirred at 80° C. for 10 hours. LCMS showed the reaction was complete. The mixture was cooled to room temperature and then poured into ice water (20 mL) at 0° C. and there was some solid formed. The solid was collected after filtration. Compound 7,8-difluoro-4-oxo-10-oxa-1-azatricyclo[7.4.1.0^{5,14}]tetradeca-2,5,7,9(14)-tetraene-3-carboxylic acid (2.1 g, 7.47 mmol, 96.23% yield) was obtained as pale yellow solid. ¹H NMR (DMSO-d₆, 400 MHz) δ 9.01 (s, 1H), 7.95 (dd, J=8.0 Hz, 2.0 Hz, 1H), 4.75 (t, J=6.0 Hz, 2H), 4.51 (t, J=7.2 Hz, 2H), 2.47-2.43 (m, 2H).

### Preparation of 10,11-difluoro-3,4,6,7-tetrahydro-[1,4]oxazepino[2,3,4-ij]quinolin-8(2H)-one (Step 4 in Scheme 8)

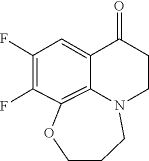 [SMILES: O=C1CCN2CCCOc3c(F)c(F)cc1c32]

To a solution of 7,8-difluoro-4-oxo-10-oxa-1-azatricyclo[7.4.1.0^{5,14}]tetradeca-2,5,7,9(14)-tetraene-3-carboxylic acid (2.1 g, 7.47 mmol, 1 eq) in MeOH (30 mL) was added NaBH₄ (1.27 g, 33.60 mmol, 4.5 eq) in portions at 0° C. during half an hour. The mixture was stirred at 20° C. for an hour under N₂ atmosphere. Then TsOH·H₂O (142.05 mg, 746.77 umol, 0.1 eq) was added and the mixture was heated at 95° C. for 10 hours under N₂ atmosphere. LCMS and TLC (petroleum ether:EtOAc=1:1, R_{f}=0.6) showed the reaction was nearly complete. The reaction mixture was concentrated under reduced pressure. The residue was quenched with water (20 mL) slowly at 0° C. and extracted with EtOAc (30 mL×3). The combined organic layer was washed with brine (20 mL×1), dried over Na₂SO₄, filtered and concentrated under reduced pressure. The residue was purified by flash silica gel chromatography (Biotage®; 20 g SepaFlash® Silica Flash Column, Eluent of 0-42% gradient ethyl acetate/petroleum ether @80 mL/min.). The eluent was removed under reduced pressure. Compound 7,8-difluoro-10-oxa-1-azatricyclo[7.4.1.0^{5,14}]tetradeca-5,7,9(14)-trien-4-one (1.2 g, 5.02 mmol, 67.17% yield) was obtained as yellow oil. ¹H NMR (DMSO-d₆, 400 MHz) δ 7.30 (dd, J=8.8 Hz, 2.0 Hz, 1H), 4.31 (t, J=6.4 Hz, 2H), 3.51˜3.44 (m, 4H), 2.60 (t, J=7.2 Hz, 2H), 2.13˜2.10 (m, 2H).

### Preparation of 10,11-difluoro-8-oxo-2,3,4,6,7,8-hexahydro-[1,4]oxazepino[2,3,4-ij]quinoline-7-carbaldehyde (Step 5 in Scheme 8)

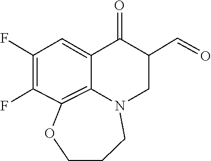 [SMILES: O=CC1CN2CCCOc3c(F)c(F)cc(c32)C1=O]

To a mixture of 7,8-difluoro-10-oxa-1-azatricyclo[7.4.1.0⁵¹⁴]tetradeca-5,7,9(14)-trien-4-one (1.1 g, 4.60 mmol, 1 eq) in DCM (30 mL) was added NaOMe (1.94 g, 17.93 mmol, 50% purity, 3.9 eq) in portions at 0° C. The mixture was stirred at 20° C. for an hour, then ethyl formate (1.33 g, 17.93 mmol, 1.44 mL, 3.9 eq) was added dropwise at 20° C. Then the mixture was stirred at 20° C. for 5 hours under N₂ atmosphere. LCMS and TLC (petroleum ether:ethyl acetate=3:1, R_{f}=0.4) showed the reaction was nearly complete. The reaction mixture was poured into ice water (20 mL) slowly at 0° C. and made pH=6 with 0.5N HCl. The organic layer was separated and the aqueous was extracted with DCM (20 mL×2). The combined organic layer was washed with brine (15 mL×1), dried over Na₂SO₄, filtered and concentrated under reduced pressure. Compound 7,8-difluoro-4-oxo-10-oxa-1-azatricyclo[7.4.1.0⁵¹⁴]tetradeca-5,7,9(14)-triene-3-carbaldehyde (1.1 g, 4.12 mmol, 89.52% yield) was obtained as pale brown oil. ¹H NMR (DMSO-d₆, 400 MHz) δ 7.76-7.74 (m, 1H), 7.32 (t, J=8.8 Hz, 1H), 4.24 (t, J=6.4 Hz, 2H), 3.50-3.43 (m, 4H), 2.10 (t, J=6.0 Hz, 2H).

### Preparation of 10,11-difluoro-8-oxo-2,3,4,8-tetrahydro-[1,4]oxazepino[2,3,4-ij]quinoline-7-carbaldehyde (Step 6 in Scheme 8)

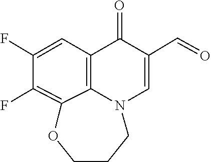 [SMILES: O=Cc1cn2c3c(c(F)c(F)cc3c1=O)OCCC2]

To a mixture of 7,8-difluoro-4-oxo-10-oxa-1-azatricyclo[7.4.1.0^{5,14}]tetradeca-5,7,9(14)-triene-3-carbaldehyde (1.2 g, 4.49 mmol, 1 eq) in MeOH (20 mL) was added MnO₂ (1.95 g, 22.45 mmol, 5 eq). Then the mixture was stirred at 20° C. for 10 hours under N₂ atmosphere. LCMS showed the reaction was complete. The reaction mixture was filtered through a pad of the Celite and the filtrate was concentrated under reduced pressure. The filter cake was washed with DCM (100 mL×2). The filtrate was concentrated under reduced pressure. Compound 7,8-difluoro-4-oxo-10-oxa-1-azatricyclo[7.4.1.0^{5,14}]tetradeca-2,5,7,9(14)-tetraene-3-carbaldehyde (0.6 g, 2.26 mmol, 50.38% yield) was obtained as off white solid. ¹H NMR (DMSO-d₆, 400 MHz) δ 10.33˜10.19 (m, 1H), 8.75˜8.61 (m, 1H), 7.87 (s, 1H), 4.83˜4.53 (m, 4H), 2.53˜2.43 (m, 2H).

### Preparation of (S)-10,11-difluoro-7-((((2-methoxypyridin-4-yl)methyl)(1-(6-nitropyridin-3-yl)piperidin-3-yl)amino)methyl)-3,4-dihydro-[1,4]oxazepino[2,3,4-ij]quinolin-8(2H)-one (Step 7 in Scheme 8)

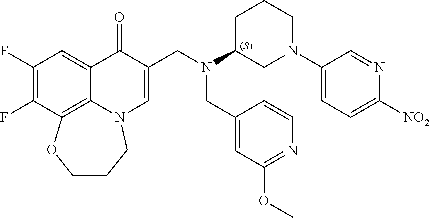

A solution of 7,8-difluoro-4-oxo-10-oxa-1-azatricyclo[7.4.1.0^{5,14}]tetradeca-2,5,7,9(14)-tetraene-3-carbaldehyde (0.2 g, 754.11 umol, 1 eq) and (3S)—N-[(2-methoxy-4-pyridyl)methyl]-1-(6-nitro-3-pyridyl)piperidin-3-amine (258.95 mg, 754.11 umol, 1 eq) in DCM (5 mL) was stirred at 20° C. for an hour. Then the mixture was cooled to 0° C., NaBH(OAc)₃ (207.77 mg, 980.35 umol, 1.3 eq) was added in portions at 0° C. The mixture was stirred at 20° C. for 10 hours. LCMS and TLC (EtOAc:MeOH=10:1, R_{f}=0.3) showed the reaction was nearly complete. The mixture was quenched with ice water (10 mL) at 0° C. and made pH=8 with sat. NaHCO₃. The mixture was extracted with DCM and i-PrOH (v:v=3:1, 10 mL×3). The combined organic layer was washed with brine (10 mL×1), dried over Na₂SO₄, filtered and concentrated under reduced pressure. Compound 7,8-difluoro-3-[[(2-methoxy-4-pyridyl)methyl-[(3S)-1-(6-nitro-3-pyridyl)-3-piperidyl]amino]methyl]-10-oxa-1-azatricyclo[7.4.1.05,14]tetradeca-2,5,7,9(14)-tetraen-4-one (0.3 g, 506.25 umol, 67.13% yield) was obtained as yellow solid. ¹H NMR (DMSO-d₆, 400 MHz) δ 8.24 (s, 1H), 8.08 (d, J=7.2 Hz, 1H), 7.99-7.96 (m, 2H), 7.66 (dd, J=8.8 Hz, 2.0 Hz, 1H), 7.44 (d, J=9.6 Hz, 1H), 7.00 (d, J=5.2 Hz, 1H), 6.82 (s, 1H), 4.58-4.26 (m, 6H), 3.78-3.76 (m, 2H), 3.75 (s, 3H), 3.68˜3.64 (m, 1H), 3.53˜3.51 (m, 1H), 3.16 (t, J=7.2 Hz, 1H), 2.96 (t, J=6.8 Hz, 1H), 2.55-2.53 (m, 1H), 2.49-2.34 (m, 2H), 2.01˜1.96 (m, 1H), 1.77-1.72 (m, 1H), 1.70˜1.63 (m, 1H), 1.44˜1.35 (m, 1H).

### Preparation of (S)-7-(((1-(6-aminopyridin-3-yl)piperidin-3-yl)((2-methoxypyridin-4-yl)methyl)amino)methyl)-10,11-difluoro-3,4-dihydro-[1,4]oxazepino[2,3,4-ij]quinolin-8(2H)-one (Step 8 in Scheme 8)

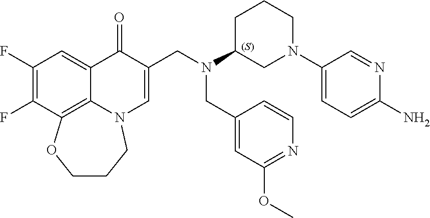

A mixture of 7,8-difluoro-3-[[(2-methoxy-4-pyridyl)methyl-[(3S)-1-(6-nitro-3-pyridyl)-3-piperidyl]amino]methyl]-10-oxa-1-azatricyclo[7.4.1.0^{5,14}]tetradeca-2,5,7,9(14)-tetraen-4-one (0.3 g, 506.25 umol, 1 eq) and Pd/C (0.1 g, 10% purity) in MeOH (30 mL) was stirred at 20° C. for 4 hours under H₂ (15 psi). LCMS and HPLC showed the reaction was complete. The mixture was filtered through a pad of the Celie. The filtrate was concentrated under reduced pressure. The residue was purified by prep-HPLC (column: Phenomenex C18 80*40 mm*3 um; mobile phase: [water (NH₄HCO₃)-MeCN]; B %: 25%-45%, 8 mins). The solvent was removed under freeze drying. Compound 3-[[[(3S)-1-(6-amino-3-pyridyl)-3-piperidyl]-[(2-methoxy-4-pyridyl)methyl]amino]methyl]-7,8-difluoro-10-oxa-1-azatricyclo[7.4.1.0^{5,14}]tetradeca-2,5,7,9(14)-tetraen-4-one (23.1 mg, 41.06 umol, 8.11% yield, 100% purity) was obtained as white solid. ¹H NMR (DMSO-d₆, 400 MHz) δ 7.97-7.95 (m, 2H), 7.68 (dd, J=8.4 Hz, 2.0 Hz, 1H), 7.61 (d, J=2.8 Hz, 1H), 7.14 (dd, J=8.8 Hz, 2.8 Hz, 1H), 6.96 (d, J=6.8 Hz, 1H), 6.77 (s, 1H), 6.37 (d, J=8.8 Hz, 1H), 5.36 (s, 2H), 4.46 (t, J=7.2 Hz, 2H), 4.42 (t, J=7.2 Hz, 2H), 3.75 (s, 3H), 3.70 (s, 2H), 3.58 (d, J=14.4 Hz, 2H), 3.32˜3.30 (m, 1H), 3.27˜3.21 (m, 1H), 2.67˜2.58 (m, 1H), 2.53˜2.51 (m, 1H), 2.49-2.35 (m, 3H), 1.98-1.95 (m, 1H), 1.77-1.74 (m, 1H), 1.55-1.42 (m, 2H). HPLC: 100% (220 nm), 100% (254 nm). MS (ESI): mass calcd. For C₃₀H₃₂F₂N₆O₃ 562.25 m/z found 563.3 [M+H]⁺.

### Compound 48

### (S)-6-(((1-(6-aminopyridin-3-yl)piperidin-3-yl)((2-methoxypyridin-4-yl)methyl)amino)methyl)-9,10-difluoro-3,3-dimethyl-2H-[1,4]oxazino[2,3,4-ij]quinolin-7(3H)-one was Prepared According to the Procedure Described Herein for Step 8 in Scheme 8

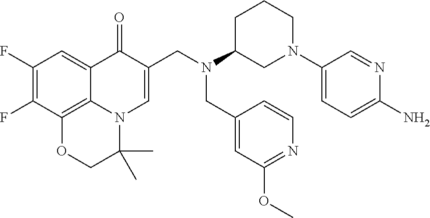 [SMILES: COc1cc(CN(Cc2cn3c4c(c(F)c(F)cc4c2=O)OCC3(C)C)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-6,7-difluoro-2,2-dimethyl-4-oxa-1-azatricyclo[7.3.1.0^{5,13}]trideca-5(13),6,8,11-tetraen-10-one (88.4 mg, 152.90 umol, 24.41% yield, 99.734% purity) was obtained as white solid. ¹H NMR (DMSO-d₆, 400 MHz) δ 8.09 (s, 1H), 7.99 (d, J=5.2 Hz, 1H), 7.60-7.55 (m, 2H), 7.14 (dd, J=8.8 Hz, 2.8 Hz, 1H), 6.96 (d, J=5.6 Hz, 1H), 6.79 (s, 1H), 6.37 (d, J=8.8 Hz, 1H), 5.37 (s, 2H), 4.30 (s, 2H), 3.76˜3.75 (m, 5H), 3.66˜3.63 (m, 2H), 3.48˜3.45 (m, 1H), 3.24˜3.21 (m, 1H), 2.82˜2.76 (m, 1H), 2.61˜2.56 (m, 1H), 2.45-2.39 (m, 1H), 1.96-1.94 (m, 1H), 1.78-1.75 (m, 1H), 1.52˜1.49 (m, 1H), 1.46 (s, 6H), 1.43˜1.42 (m, 1H). LCMS: 99.73% (220 nm), 99.65% (254 nm). MS (ESI): mass calcd. For C₃₁H₃₄F₂N₆O₃ 576.27 m/z found 577.4 [M+H]⁺.

### Compound 49

### (S)-6-(((1-(6-aminopyridin-3-yl)piperidin-3-yl)((2-methoxypyridin-4-yl)methyl)amino)methyl)-9,10-difluorospiro[[1,4]oxazino[2,3,4-ij]quinoline-2,1′-cyclobutan]-7(3H)-one was Prepared According to the Procedure Described Herein for Step 8 in Scheme 8

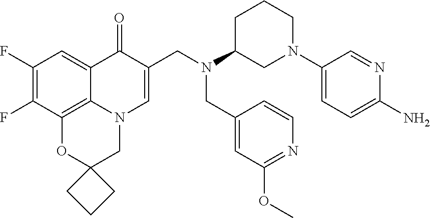 [SMILES: COc1cc(CN(Cc2cn3c4c(c(F)c(F)cc4c2=O)OC2(CCC2)C3)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-6,7-difluoro-spiro[4-oxa-1-azatricyclo[7.3.1.05,13]trideca-5(13),6,8,11-tetraene-3,1′-cyclobutane]-10-one (25 mg, 40.33 umol, 10.85% yield, 94.13% purity) was obtained as pale yellow solid. ¹H NMR (DMSO-d₆, 400 MHz) δ 7.99 (d, J=5.6 Hz, 2H), 7.60 (d, J=2.8 Hz, 1H), 7.52 (dd, J=8.0 Hz, 10.8 Hz, 1H), 7.14 (dd, J=3.2 Hz, 8.8 Hz, 1H), 6.99 (d, J=5.6 Hz, 1H), 6.80 (s, 1H), 6.37 (d, J=8.8 Hz, 1H), 5.37 (s, 2H), 4.37 (d, J=3.2 Hz, 2H), 3.76 (s, 3H), 3.72 (s, 2H), 3.61 (d, J=8.4 Hz, 2H), 3.50-3.47 (m, 1H), 3.25-3.20 (m, 1H), 2.79-2.73 (m, 1H), 2.61˜2.58 (m, 1H), 2.41˜2.38 (m, 2H), 2.29-2.25 (m, 2H), 2.12˜2.07 (m, 2H), 1.98-1.96 (m, 2H), 1.77-1.74 (m, 1H), 1.53˜1.41 (m, 2H). LCMS: 94.13% (220 nm), 99.37% (254 nm). MS (ESI): mass calcd. For C₃₂H₃₄F₂N₆O₃ 588.27 m/z found 589.4 [M+H]⁺.

### Compound 50

### (S)-6-(((1-(6-aminopyridin-3-yl)piperidin-3-yl)((2-methoxypyridin-4-yl)methyl)amino)methyl)-9,10-difluoro-2,2-dimethyl-2H-[1,4]oxazino[2,3,4-ij]quinolin-7(3H)-one was Prepared According to the Procedure Described Herein for Step 8 in Scheme 8

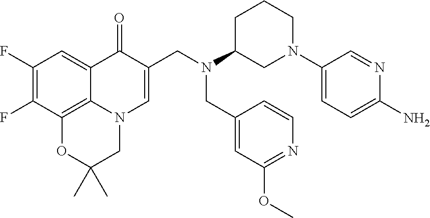 [SMILES: COc1cc(CN(Cc2cn3c4c(c(F)c(F)cc4c2=O)OC(C)(C)C3)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-6,7-difluoro-3,3-dimethyl-4-oxa-1-azatricyclo[7.3.1.0^{5,13}]trideca-5(13),6,8,11-tetraen-10-one (123.9 mg, 213.99 umol, 32.45% yield, 99.59% purity) was obtained as pale yellow solid. ¹H NMR (DMSO-d₆, 400 MHz) δ 7.97 (d, J=5.2 Hz, 1H), 7.91 (s, 1H), 7.60 (d, J=2.8 Hz, 1H), 7.51 (dd, J=8.0 Hz, 10.8 Hz, 1H), 7.13 (dd, J=2.8 Hz, 8.8 Hz, 1H), 6.97 (d, J=5.2 Hz, 1H), 6.77 (s, 1H), 6.38 (d, J=8.4 Hz, 1H), 5.37˜5.35 (m, 2H), 4.20˜4.12 (m, 2H), 3.75 (s, 3H), 3.72 (s, 2H), 3.66-3.61 (m, 2H), 3.46 (d, J=9.6 Hz, 1H), 3.24 (d, J=11.6 Hz, 1H), 2.79-2.73 (m, 1H), 2.58 (t, J=10.8 Hz, 1H), 2.41 (t, J=11.6 Hz, 1H), 2.00 (d, J=10.4 Hz, 1H), 1.77 (d, J=12.8 Hz, 1H), 1.51˜1.43 (m, 2H), 1.38 (d, J=2.8 Hz, 6H). LCMS: 99.59% (220 nm), 99.61% (254 nm). MS (ESI): mass calcd. For C₃₁H₃₄F₂N₆O₃ 576.27 m/z found 577.4 [M+H]⁺.

### Compound 51

### 6-((((S)-1-(6-aminopyridin-3-yl)piperidin-3-yl)((2-methoxypyridin-4-yl)methyl)amino)methyl)-9,10-difluoro-2-methyl-2H-[1,4]oxazino[2,3,4-ij]quinolin-7(3H)-one was Prepared According to the Procedure Described Herein for Step 8 in Scheme 8

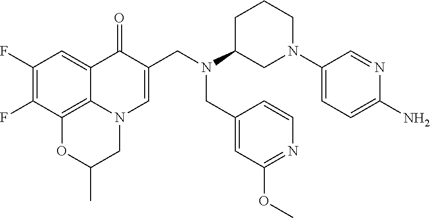 [SMILES: COc1cc(CN(Cc2cn3c4c(c(F)c(F)cc4c2=O)OC(C)C3)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-6,7-difluoro-3-methyl-4-oxa-1-azatricyclo[7.3.1.0^{5,13}]trideca-5(13),6,8,11-tetraen-10-one (94.2 mg, 156.02 umol, 23.11% yield, 93.18% purity) was obtained as yellow solid. ¹H NMR (DMSO-d₆, 400 MHz) δ 8.00 (d, J=5.2 Hz, 1H), 7.92 (s, 1H), 7.61 (s, 1H), 7.52 (dd, J=8.4 Hz, 10.8 Hz, 1H), 7.13 (dd, J=2.8 Hz, 9.2 Hz, 1H), 7.00 (d, J=5.2 Hz, 1H), 6.79 (s, 1H), 6.39 (d, J=8.8 Hz, 1H), 5.36˜5.34 (m, 2H), 4.61˜4.57 (m, 1H), 4.51˜4.46 (m, 1H), 3.99˜3.93 (m, 1H), 3.82˜3.81 (m, 1H), 3.80 (s, 3H), 3.72 (s, 1H), 3.72˜3.60 (m, 2H), 3.49 (d, J=11.2 Hz, 1H), 3.25 (d, J=11.6 Hz, 1H), 2.77-2.72 (m, 1H), 2.60-2.59 (m, 1H), 2.41˜2.38 (m, 1H), 1.97-1.95 (m, 1H), 1.76-1.73 (m, 1H), 1.48-1.40 (m, 5H). LCMS: 93.18% (220 nm), 95.64% (254 nm). MS (ESI): mass calcd. For C₃₀H₃₂F₂N₆O₃ 562.25 m/z found 563.4 [M+H]⁺.

### Compound 52

### 6-((((S)-1-(6-aminopyridin-3-yl)piperidin-3-yl)((2-methoxypyridin-4-yl)methyl)amino)methyl)-8,9-difluoro-3-methyl-2H-[1,4]oxazino[2,3,4-ij]quinolin-7(3H)-one was Prepared According to the Procedure Described Herein for Step 8 in Scheme 8

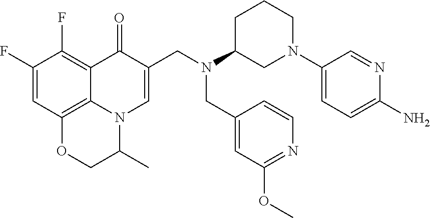 [SMILES: COc1cc(CN(Cc2cn3c4c(cc(F)c(F)c4c2=O)OCC3C)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-7,8-difluoro-2-methyl-4-oxa-1-azatricyclo[7.3.1.0¹³]trideca-5(13),6,8,11-tetraen-10-one (143.4 mg, 252.53 umol, 37.41% yield, 99.077% purity) was obtained as off-white solid. ¹H NMR (DMSO-d₆, 400 MHz) δ 7.99-7.96 (m, 2H), 7.61 (d, J=2.4 Hz, 1H), 7.41 (dd, J=11.6 Hz, 6.8 Hz, 1H), 7.15 (dd, J=8.4 Hz, 2.4 Hz, 1H), 6.99 (d, J=5.2 Hz, 1H), 6.81 (s, 1H), 6.38 (dd, J=9.2 Hz, 3.2 Hz, 1H), 5.37-5.34 (m, 2H), 4.62˜4.60 (m, 1H), 4.39 (d, J=10.8 Hz, 1H), 4.25 (d, J=9.2 Hz, 1H), 3.75 (s, 3H), 3.72˜3.71 (m, 2H), 3.67˜3.54 (m, 2H), 3.53˜3.50 (m, 1H), 2.25-2.22 (m, 1H), 2.76-2.73 (m, 1H), 2.61˜2.55 (m, 1H), 2.44-2.39 (m, 1H), 1.99-1.96 (m, 1H), 1.78-1.75 (m, 1H), 1.54-1.39 (m, 2H), 1.29 (dd, J=6.4 Hz, 4.8 Hz, 3H). HPLC: 99.08% (220 nm), 98.83% (254 nm). MS (ESI): mass calcd. For C₃₀H₃₂F₂N₆O₃ 562.25 m/z found 563.4 [M+H]⁺.

### Compound 53

### 6-((((S)-1-(6-aminopyridin-3-yl)piperidin-3-yl)((2-methoxypyridin-4-yl)methyl)amino)methyl)-9,10-difluoro-3-isopropyl-2H-[1,4]oxazino[2,3,4-ij]quinolin-7(3H)-one was Prepared According to the Procedure Described Herein for Step 8 in Scheme 8

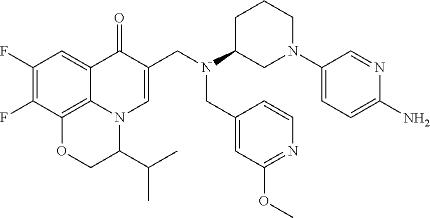 [SMILES: COc1cc(CN(Cc2cn3c4c(c(F)c(F)cc4c2=O)OCC3C(C)C)[C@H]2CCCN(c3ccc(N)nc3)C2)ccn1]

11-[[[(3S)-1-(6-amino-3-pyridyl)-3-piperidyl]-[(2-methoxy-4-pyridyl)methyl]amino]methyl]-6,7-difluoro-2-isopropyl-4-oxa-1-azatricyclo[7.3.1.05,13]trideca-5(13),6,8,11-tetraen-10-one (136.8 mg, 228.50 umol, 19.70% yield, 98.66% purity) was obtained as pale yellow solid. ¹H NMR (DMSO-d₆, 400 MHz) δ 8.01 (d, J=5.6 Hz, 1H), 7.60 (dd, J=3.2 Hz, 9.6 Hz, 1H), 7.55 (dd, J=8.4 Hz, 10.0 Hz, 1H), 7.14-7.12 (m, 1H), 7.0.3˜7.01 (m, 1H), 6.84 (s, 1H), 6.37 (dd, J=4.8 Hz, 8.8 Hz, 1H), 5.37 (d, J=5.2 Hz, 2H), 4.88 (d, J=12.0 Hz, 1H), 4.30-4.30 (m, 2H), 3.78-3.74 (m, 5H), 3.65 (d, J=13.6 Hz, 1H), 3.52 (t, J=14.0 Hz, 2H), 3.24-3.20 (m, 1H), 2.76-2.70 (m, 1H), 2.59 (t, J=10.8 Hz, 1H), 2.45-2.37 (m, 1H), 3.62˜3.61 (m, 1H), 2.00-1.91 (m, 2H), 1.75-1.74 (m, 1H), 1.48-1.41 (m, 2H), 0.99 (dd, J=6.8 Hz, 12.0 Hz, 3H), 0.74 (dd, J=6.8 Hz, 11.2 Hz, 3H). LCMS: 98.66% (220 nm), 99.01% (254 nm). MS (ESI): mass calcd. For C₃₂H₃₆F₂N₆O₃ 590.28 m/z found 591.4 [M+H]⁺.

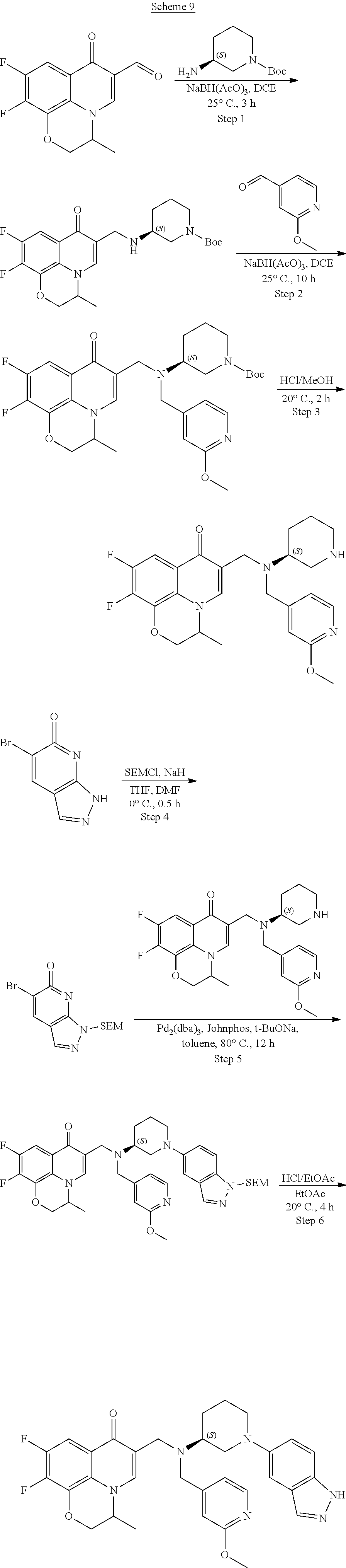

### Specific Procedures for Preparing Compounds in Scheme 9

### Compound 54

### Preparation of (3S)-tert-butyl 3-(((9,10-difluoro-3-methyl-7-oxo-3,7-dihydro-2H-[1,4]oxazino[2,3,4-ij]quinolin-6-yl)methyl)amino)piperidine-1-carboxylate (Step 1 in Scheme 2)

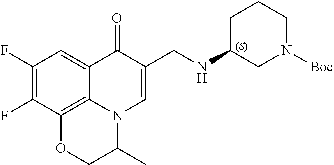

To a mixture of 6,7-difluoro-2-methyl-10-oxo-4-oxa-1-azatricyclo[7.3.1.05,13]trideca-5 (13),6,8,11-tetraene-11-carbaldehyde (1.5 g, 5.66 mmol, 1 eq) and tert-butyl(3S)-3-amino piperidine-1-carboxylate (1.25 g, 6.22 mmol, 1.1 eq) in DCE (15 mL) was added NaBH(OAc)₃ (1.80 g, 8.48 mmol, 1.5 eq) at 25° C. under N₂. The mixture was stirred at 25° C. for 3 hours. LCMS showed 6,7-difluoro-2-methyl-10-oxo-4-oxa-1-azatricyclo[7.3.1.05,13]trideca-5(13),6,8,11-tetraene-11-carbaldehyde was consumed completely and one main peak with desired mass was detected. The reaction mixture was quenched by addition water (50 mL) at 25° C. and then extracted with EtOAc (40 mL×3). The combined organic layers were washed with brine (30 mL×2), dried over Na₂SO₄, filtered and concentrated under reduced pressure. Compound tert-butyl(3S)-3-[(6,7-difluoro-2-methyl-10-oxo-4-oxa-1-azatricyclo[7.3.1.05,13]trideca-5(13),6,8,11-tetraen-11-yl)methylamino]piperidine-1-carboxylate (2.5 g, crude) was obtained as brown solid. ¹H NMR (DMSO-d₆, 400 MHz) δ 8.07 (d, J=5.2 Hz, 1H), 7.58 (dd, J=10.4 Hz, 4.4 Hz, 1H), 4.64 (s, 1H), 4.57 (d, J=11.6 Hz, 1H), 4.40 (d, J=10.8 Hz, 1H), 3.90 (s, 1H), 3.71 (s, 1H), 3.69 (s, 1H), 3.39˜3.29 (m, 2H), 2.88˜2.82 (m, 2H), 1.40˜1.38 (m, 4H), 1.37 (s, 9H), 1.30 (s, 3H).

### Preparation of (3S)-tert-butyl 3-(((9,10-difluoro-3-methyl-7-oxo-3,7-dihydro-2H-[1,4]oxazino[2,3,4-ij]quinolin-6-yl)methyl)((2-methoxypyridin-4-yl)methyl)amino)piperidine-1-carboxylate (Step 2 in Scheme 9)

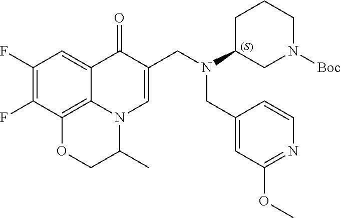

To a mixture of tert-butyl (3S)-3-[(6,7-difluoro-2-methyl-10-oxo-4-oxa-1-azatricyclo [7.3.1.05,13]trideca-5(13),6,8,11-tetraen-11-yl)methylamino]piperidine-1-carboxylate (2.5 g, 5.56 mmol, 1 eq) and 2-methoxypyridine-4-carbaldehyde (762.73 mg, 5.56 mmol, 1 eq) in DCE (25 mL) was added NaBH(OAc)₃ (1.77 g, 8.34 mmol, 1.5 eq) at 25° C. under N₂. Then the mixture was stirred at 25° C. for 4 hours. LCMS showed tert-butyl (3S)-3-[(6,7-difluoro-2-methyl-10-oxo-4-oxa-1-azatricyclo[7.3.1.05,13]trideca-5(13),6,8,11-tetraen-11-yl)methylamino]piperidine-1-carboxylate was consumed completely and one new peak with desired mass was detected. The reaction mixture was quenched by addition water (50 mL) at 25° C. and extracted with DCM (30 mL×3). The combined organic layers were washed with brine (30 mL×2), dried over Na₂SO₄, filtered and concentrated under reduced pressure. Compound tert-butyl (3S)-3-[(6,7-difluoro-2-methyl-10-oxo-4-oxa-1-azatricyclo[7.3.1.05,13]trideca-5(13),6,8,11-tetraen-11-yl)methyl-[(2-methoxy-4-pyridyl)methyl]amino]piperidine-1-carboxylate (3.1 g, crude) was obtained as brown solid. ¹H NMR (DMSO-d₆, 400 MHz) δ 8.07 (d, J=5.6 Hz, 1H), 8.01 (d, J=4.4 Hz, 1H), 7.54 (t, J=8.4 Hz, 1H), 6.94 (d, J=5.2 Hz, 1H), 6.76 (s, 1H), 4.66 (t, J=6.8 Hz, 1H), 4.55 (d, J=10.8 Hz, 1H), 4.38 (d, J=11.6 Hz, 1H), 3.91 (d, J=11.2 Hz, 1H), 3.80 (s, 2H), 3.75 (s, 3H), 3.70 (s, 2H), 3.60 (s, 2H), 2.67˜2.57 (m, 2H), 1.67 (d, J=12.0 Hz, 2H), 1.54˜1.45 (m, 2H), 1.37 (s, 3H), 1.34 (s, 9H).

### Preparation of 9,10-difluoro-6-((((2-methoxypyridin-4-yl)methyl)((S)-piperidin-3-yl)amino)methyl)-3-methyl-2H-[1,4]oxazino[2,3,4-ij]quinolin-7(3H)-one (Step 3 in Scheme 9)

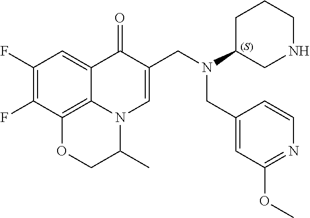

A solution of tert-butyl(3S)-3-[(6,7-difluoro-2-methyl-10-oxo-4-oxa-1-azatricyclo [7.3.1.05,13]trideca-5(13),6,8,11-tetraen-11-yl)methyl-[(2-methoxy-4-pyridyl)methyl]amino]piperidine-1-carboxylate (3.1 g, 5.43 mmol, 1 eq) in HCl/MeOH (4 M, 30 mL) was stirred at 20° C. for 2 hours. LC-MS showed the reaction was complete. The mixture was concentrated under reduced pressure and then made pH=7 with sat. NaHCO₃. The aqueous phase was dried under freeze drying. Compound 6,7-difluoro-11-[[(2-methoxy-4-pyridyl)methyl-[(3S)-3-piperidyl]amino]methyl]-2-methyl-4-oxa-1-azatricyclo[7.3.1.05,13]trideca-5(13),6,8,11-tetraen-10-one (1.5 g, crude) was obtained as brown oil. ¹H NMR (DMSO-d₆, 400 MHz) δ 8.15 (t, J=3.2 Hz, 1H), 7.62˜7.60 (m, 1H), 7.35 (dd, J=5.6 Hz, 11.2 Hz, 1H), 7.24 (d, J=8.0 Hz, 1H), 7.03 (d, J=5.6 Hz, 1H), 4.66-4.61 (m, 1H), 4.57-4.55 (m, 2H), 4.39 (d, J=13.6 Hz, 2H), 4.31˜4.09 (m, 2H), 3.91 (s, 3H), 2.82 (s, 2H), 3.66˜3.56 (m, 1H), 3.37˜3.10 (m, 1H), 3.21˜3.20 (m, 1H), 1.99-1.90 (m, 2H), 1.76-1.59 (m, 2H), 1.46 (dd, J=14.4 Hz, 6.8 Hz, 3H).

### Preparation of 5-bromo-1-((2-(trimethylsilyl)ethoxy)methyl)-1H-pyrazolo[3,4-b]pyridine (Step 4 in Scheme 9)

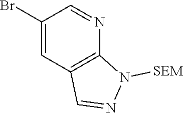 [SMILES: C[Si](C)(C)CCOCn1ncc2cc(Br)cnc21]

To a solution of 5-bromo-1H-pyrazolo[3,4-b]pyridine (2 g, 10.10 mmol, 1 eq) in THF (18 mL) and DMF (2 mL) was added NaH (403.96 mg, 20.20 mmol, 60% purity, 2 eq) in portions at 0° C. The mixture was stirred at 0° C. for half an hour. Then SEM-Cl (2.18 g, 13.12 mmol, 2.32 mL, 1.3 eq) was added dropwise slowly a 0° C. LCMS showed the reaction was complete. The reaction mixture was poured into ice water (50 mL) slowly at 0° C. and then extracted with EtOAc (15 mL×3). The combined organic phase was washed with brine (10 mL×1), dried over anhydrous Na₂SO₄, filtered and concentrated in vacuum. The residue was purified by flash silica gel chromatography (Biotage; 20 g SepaFlash® Silica Flash Column, Eluent of 0˜37% Petroleum ether/Ethyl acetate gradient @60 mL/min). The eluent was removed under reduced pressure. Compound 2-[(5-bromopyrazolo[3,4-b]pyridin-1-yl)methoxy]ethyl-trimethyl-silane (0.73 g, 2.22 mmol, 21.97% yield) was obtained as white solid. ¹H NMR (CDCl₃, 400 MHz) δ 8.66 (d, J=2.4 Hz, 1H), 8.61 (s, 1H), 8.58 (d, J=2.4 Hz, 1H), 5.77 (s, 2H), 3.64 (t, J=8.0 Hz, 2H), 0.87 (t, J=8.0 Hz, 2H), −0.06 (s, 9H).

### Preparation of 9,10-difluoro-6-((((2-methoxypyridin-4-yl)methyl)((S)-1-(1-((2-(trimethylsilyl)ethoxy)methyl)-1H-pyrazolo[3,4-b]pyridin-5-yl)piperidin-3-yl)amino)methyl)-3-methyl-2H-[1,4]oxazino[2,3,4-ij]quinolin-7(3H)-one (Step 5 in Scheme 9)

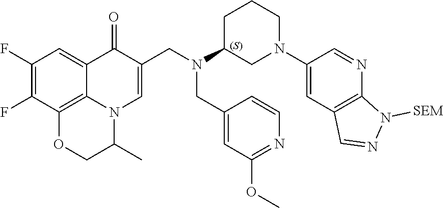

A mixture of 6,7-difluoro-11-[[(2-methoxy-4-pyridyl)methyl-[(3S)-3-piperidyl]amino]ethyl]2-ethyl4-oxa-1-azatricyclo[7.3.1.0^{5,13}]trideca-5(13),6,8,11-tetraen-10-one (322.48 mg, 685.39 umol, 1.5 eq), 2-[(5-bromopyrazolo[3,4-b]pyridin-1-yl)methoxy]ethyl-trimethyl-silane (150 mg, 456.93 umol, 1 eq), Pd₂(dba)₃ (41.84 mg, 45.69 umol, 0.1 eq), JohnPhos (27.27 mg, 91.39 umol, 0.2 eq) and t-BuONa (87.82 mg, 913.86 umol, 2 eq) in toluene (3 mL) was degassed and purged with N₂ for 3 times and then the mixture was stirred at 80° C. for 12 hours under N₂ atmosphere. LCMS showed ˜12% of 6,7-difluoro-11-[[(2-methoxy-4-pyridyl)methyl-[(3S)-3-iperidyl]mino]methyl]-2-methyl-4-oxa-1-azatricyclo[7.3.1.0^{5,13}]trideca-5(13),6,8,11-tetraen-10-one was remained. Several new peaks were shown on LC-MS and ˜20% of desired product was detected. The reaction mixture was concentrated under reduced pressure. The residue was purified by flash silica gel chromatography (Biotage; 40 SepaFlash® Silica Flash Column, Eluent of 0˜5% Ethyl acetate/Methanol gradient @40 mL/min). The eluent was removed under reduced pressure. Compound 6,7-difluoro-11-[[(2-methoxy-4-pyridyl)methyl-[(3S)-1-[1-2-trimethylilylethoxy methyl)pyrazolo[3,4-b]pyridin-5-yl]-3-piperidyl]amino]methyl]-2-methyl-4-oxa-1-azatricyclo[7.3.1.0^{5,13}]trideca-5(13),6,8,11-tetraen-10-one (260 mg, 362.18 umol, 39.63% yield) was obtained as brown solid. ¹H NMR (CDCl₃, 400 MHz) δ 8.61 (d, J=2.0 Hz, 1H), 8.31 (d, J=5.6 Hz, 1H), 8.14 (d, J=11.6 Hz, 1H), 8.01 (d, J=5.2 Hz, 1H), 7.57-7.51 (m, 1H), 7.37-7.36 (m, 1H), 7.04-7.03 (m, 1H), 6.87-6.86 (m, 1H), 5.66 (s, 2H), 4.73˜4.69 (m, 1H), 4.58˜4.55 (m, 1H), 4.42˜4.39 (m, 1H), 3.86˜3.58 (m, 12H), 2.83˜2.82 (m, 1H), 2.74˜2.67 (m, 1H), 2.04˜2.00 (m, 1H), 1.85˜1.81 (m, 1H), 1.62˜1.47 (m, 2H), 1.33 (t, J=6.4 Hz, 3H), 0.86 (t, J=8.0 Hz, 2H), −0.06 (s, 9H).

### Preparation of 6-((((S)-1-(1H-pyrazolo[3,4-b]pyridin-5-yl)piperidin-3-yl)((2-methoxypyridin-4-yl)methyl)amino)methyl)-9,10-difluoro-3-methyl-2H-[1,4]oxazino[2,3,4-ij]quinolin-7(3H)-one (Step 6 in Scheme 9)

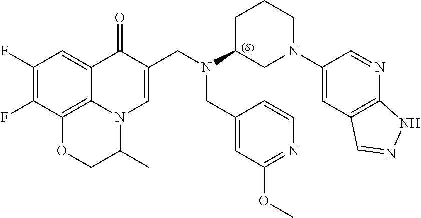

To a solution of 6,7-difluoro-11-[[(2-methoxy-4-pyridyl)methyl-[(3S)-1-[1-(2-trimethyl ilylethoxymethyl)pyrazolo[3,4-b]pyridin-5-yl]-3-piperidyl]amino]methyl]-2-methyl-4-oxa-1-azatricyclo[7.3.1.0^{5,13}]trideca-5(13),6,8,11-tetraen-10-one (0.25 g, 348.25 umol, 1 eq) in EtOAc (1 mL) was added HCl/EtOAc (4 M, 8 mL). The mixture was stirred at 20° C. for 4 hours. LCMS showed the reaction was complete. The reaction mixture was concentrated under reduced pressure. The residue was dissolved in MeOH (2 mL) and NH₃·H₂O (0.01 mL) and then concentrated under reduced pressure to give 0.22 g of residue. 90 mg of the residue was purified by prep-HPLC (column: Phenomenex C18 75*30 mm*3 um; mobile phase: [water (NH₄HCO₃)-MeCN]; B %: 30%-50%, 8 mins) and (column: Waters Xbridge BEH C18 100*30 mm*10 um; mobile phase: [water (NH₄HCO₃)-MeCN]; B %: 40%-50%, 8 min) for two times. The eluent was removed under freeze drying. Compound 6,7-difluoro-11-[[(2-methoxy-4-pyridyl)methyl-[(3S)-1-(1H-pyrazolo[3,4-b]pyridin-5-yl)-3-piperidyl]amino]methyl]-2-methyl-4-oxa-1-azatricyclo[7.3.1.0^{5,13}]trideca-5(13),6,8,11-tetraen-10-one (7.4 mg, 12.59 umol, 3.62% yield, 100% purity) was obtained as white solid. ¹H NMR (DMSO-d₆, 400 MHz) δ 13.35 (m, 1H), 8.43 (d, J=2.4 Hz, 1H), 8.13 (d, J=12.0 Hz, 1H), 8.01 (d, J=9.2 Hz, 1H), 7.95 (d, J=6.4 Hz, 1H), 7.62˜7.52 (m, 2H), 7.03˜7.02 (m, 1H), 6.86 (d, J=2.4 Hz, 1H), 4.74-4.68 (m, 1H), 4.57 (d, J=11.2 Hz, 1H), 4.40˜4.37 (m, 1H), 3.86˜3.79 (m, 1H), 3.76 (s, 3H), 3.75 (d, J=5.6 Hz, 1H), 3.71˜3.57 (m, 2H), 3.53˜3.51 (m, 1H), 3.37˜3.33 (m, 1H), 2.89˜2.72 (m, 2H), 2.62˜2.57 (m, 1H), 2.03˜1.97 (m, 1H), 1.84˜1.81 (m, 1H), 1.58-1.47 (m, 2H), 1.33 (dd, J=4.4 Hz, 6.4 Hz, 3H). LCMS: 100% (220 nm), 100% (254 nm). MS (ESI): mass calcd. For C₃₁H₃₁F₂N₇O₃ 587.25 m/z found 588.3 [M+H]⁺.

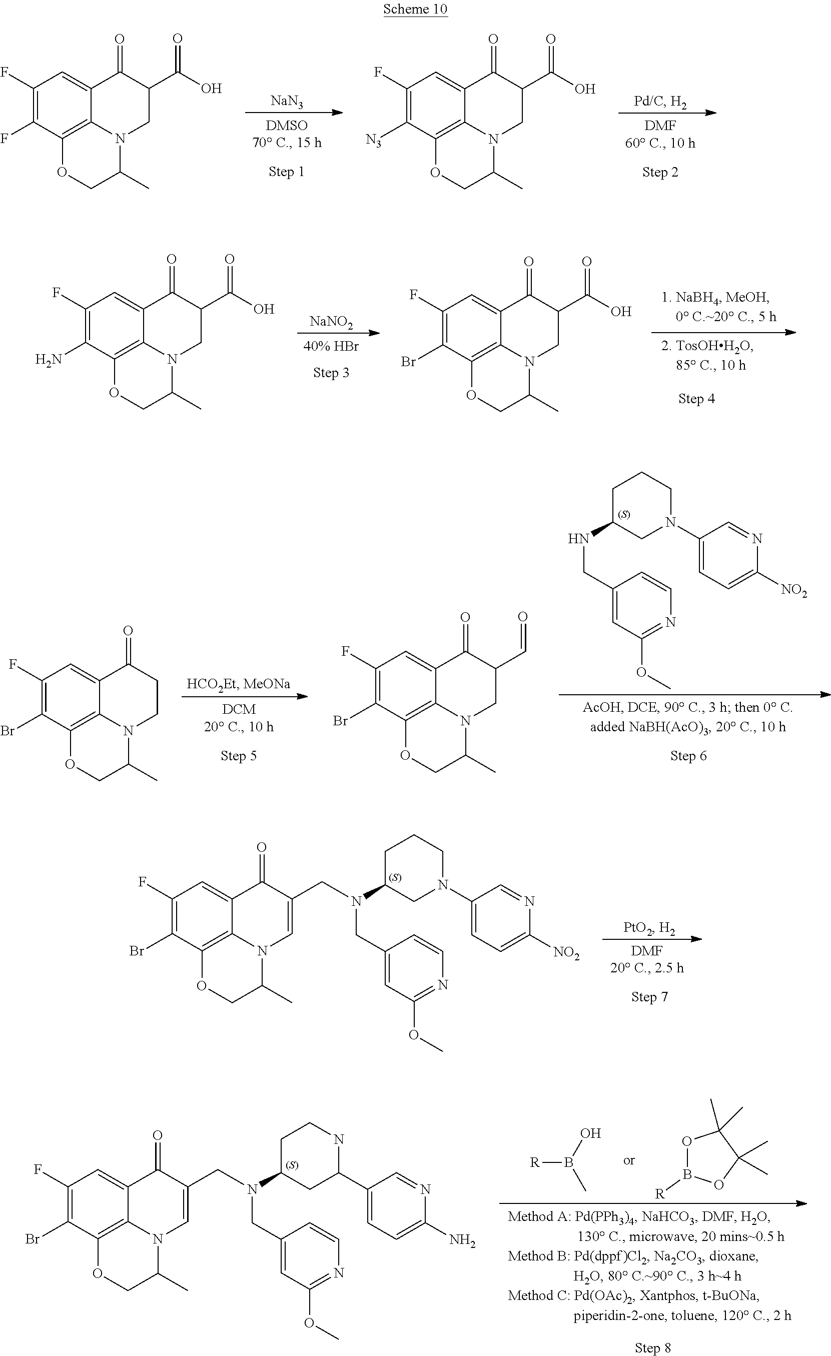

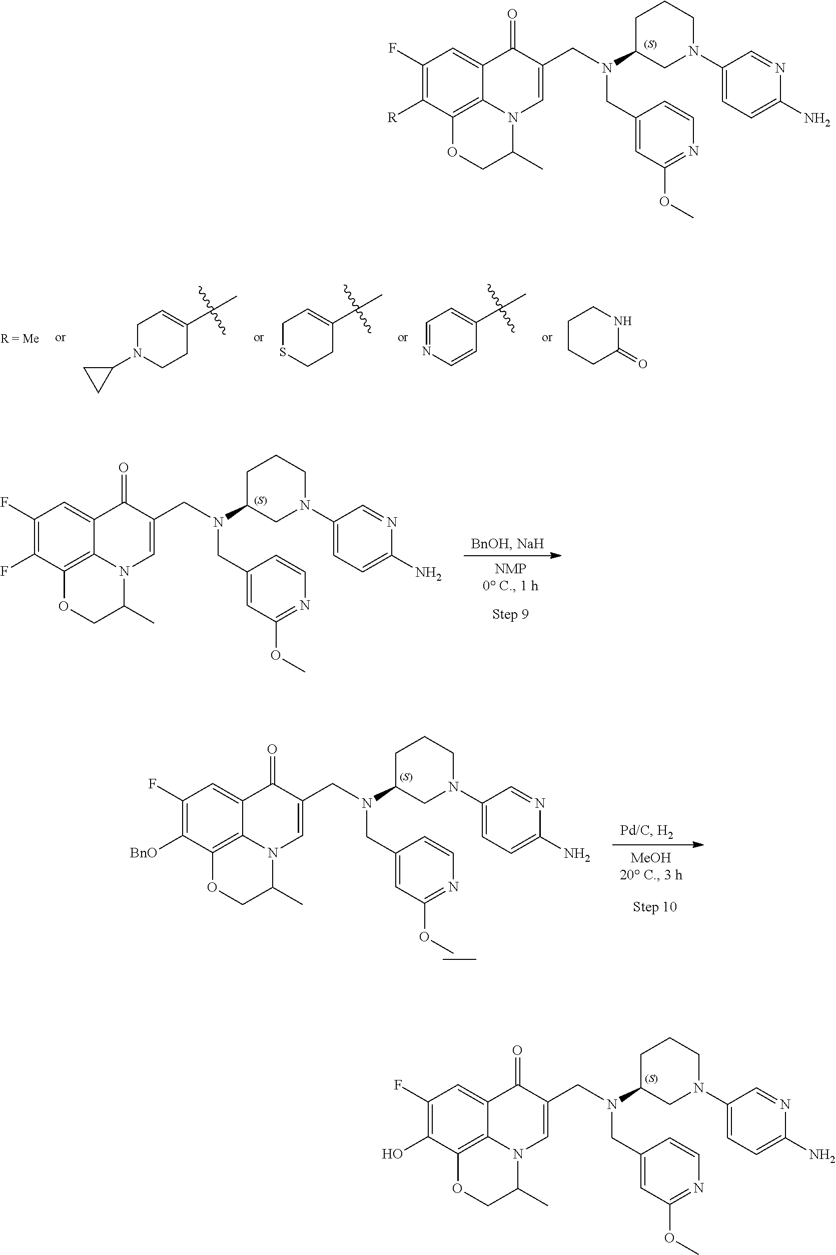

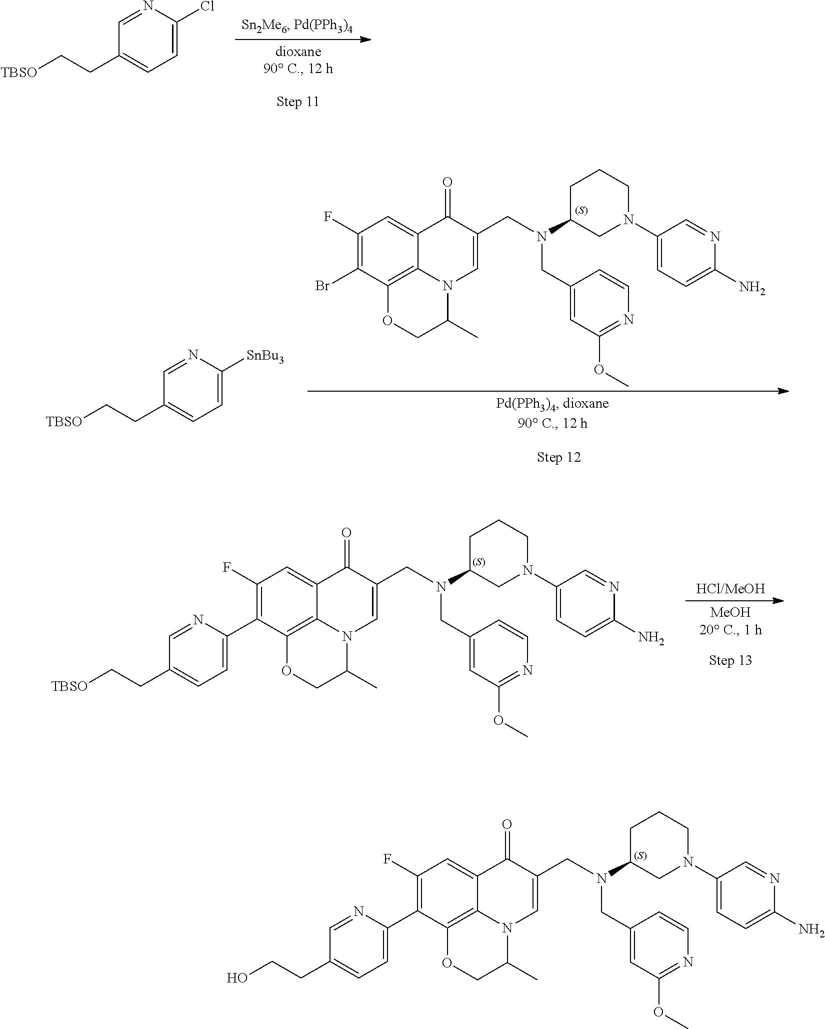

### General Procedures for Preparing Compounds in Scheme 10

### Compound 55

### Preparation of 10-azido-9-fluoro-3-methyl-7-oxo-3,7-dihydro-2H-[1,4]oxazino[2,3,4-ij]quinoline-6-carboxylic Acid (Step 1 in Scheme 10)

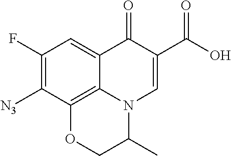 [SMILES: CC1COc2c(N=[N+]=[N-])c(F)cc3c(=O)c(C(=O)O)cn1c23]

To a solution of 6,7-difluoro-2-methyl-10-oxo-4-oxa-1-azatricyclo[7.3.1.05,13]trideca-5(13),6,8,11-tetraene-11-carboxylic acid (25 g, 88.90 mmol, 1 eq) in DMSO (200 mL) was added NaN₃ (8.10 g, 124.60 mmol, 1.4 eq) in portions at 35° C. under N₂. Then the mixture was stirred at 70° C. for 15 hours. LCMS showed the reaction was nearly complete. The mixture was cooled to 20° C. and poured into ice water (800 mL) slowly. There was some precipitate formed. The solid was collected after filtration and washed with water (800 mL), then concentrated under reduced pressure at 35° C. Compound 6-azido-7-fluoro-2-methyl-10-oxo-4-oxa-1-azatricyclo [7.3.1.05,13]trideca-5(13),6,8,11-tetraene-11-carboxylic acid (25 g, 82.17 mmol, 92.43% yield) was obtained as off white solid. ¹H NMR (DMSO-d₆, 400 MHz) δ 14.95 (s, 1H), 9.00 (s, 1H), 7.70 (d, J=10.8 Hz, 1H), 4.98-4.96 (m, 1H), 4.70˜4.67 (m, 1H), 4.50˜4.46 (m, 1H), 1.46 (d, J=6.8 Hz, 3H).

### Preparation of 10-amino-9-fluoro-3-methyl-7-oxo-3,7-dihydro-2H-[1,4]oxazino[2,3,4-ij]quinoline-6-carboxylic Acid (Step 2 in Scheme 10)

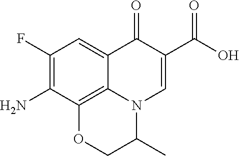 [SMILES: CC1COc2c(N)c(F)cc3c(=O)c(C(=O)O)cn1c23]

To a solution of 6-azido-7-fluoro-2-methyl-10-oxo-4-oxa-1-azatricyclo[7.3.1.05,13]trideca-5(13),6,8,11-tetraene-11-carboxylic acid (20 g, 65.74 mmol, 1 eq) in DMF (500 mL) was added Pd/C (5 g, 10% purity) under argon. The suspension was degassed under vacuum and purged with H₂ several times. The mixture was stirred under H₂ (30 psi) at 60° C. for 10 hours. Three batches were carried out and workup together. LCMS and HPLC indicated the reaction was nearly completed. The reaction mixture was cooled to room temperature, filtered through celite and washed with DMF (1000 mL) and MeOH (500 mL). The filtrate was concentrated under reduced pressure. The crude product was triturated with MeOH (200 mL) at 20° C. for 10 mins. Compound 6-amino-7-fluoro-2-methyl-10-oxo-4-oxa-1-azatricyclo [7.3.1.05,13]trideca-5(13),6,8,11-tetraene-11-carboxylic acid (49 g, 176.11 mmol, 89.30% yield) was obtained as gray solid. ¹H NMR (DMSO-d₆, 400 MHz) δ 15.61 (s, 1H), 8.87 (s, 1H), 7.55 (d, J=11.6 Hz, 1H), 6.24 (s, 2H), 4.91˜4.86 (m, 1H), 4.55-4.52 (m, 1H), 4.35-4.32 (m, 1H), 1.45 (d, J=6.8 Hz, 3H).

### Preparation of 10-bromo-9-fluoro-3-methyl-7-oxo-3,7-dihydro-2H-[1,4]oxazino[2,3,4-ij]quinoline-6-carboxylic Acid (Step 3 in Scheme 10)

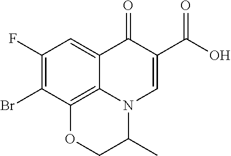 [SMILES: CC1COc2c(Br)c(F)cc3c(=O)c(C(=O)O)cn1c23]

HBr (465.29 g, 2.30 mol, 312.27 mL, 40% purity, 40 eq) was cooled to 0° C. and 6-amino-7-fluoro-2-methyl-10-oxo-4-oxa-1-azatricyclo[7.3.1.05,13]trideca-5(13),6,8,11-tetraene-11-carboxylic acid (16 g, 57.51 mmol, 1 eq) was added. Then a solution of NaNO₂ (4.76 g, 69.01 mmol, 1.2 eq) in H₂O (9 mL) was added dropwise at 0° C.-5° C. during half an hour. The mixture was stirred at 0° C. for half an hour under N₂ atmosphere. LCMS showed 6-amino-7-fluoro-2-methyl-10-oxo-4-oxa-1-azatricyclo[7.3.1.05,13]trideca-5(13),6,8,11-tetraene-11-carboxylic acid was consumed, -59% of P1 and ˜33% of side product (NH₂ turned to H, MS=264, also contained 282 which is di-F acid material from the above two step, which is confirmed by FNMR) were detected on LCMS. The mixture was poured into ice water (1 L) slowly. There was some precipitate formed. The solid was collected after filtration and washed with water (100 mL), then concentrated under reduced pressure. The filtrate was made pH=7 with 2N NaOH and discarded. The obtained solid was partitioned in H₂O (150 mL) and made pH=7-8 with sat NaHCO₃. The solid was collected after filtration. The obtained solid was partitioned in H₂O (150 mL) and made pH=4 with 1N HCl. The solid was collected after filtration and concentration under reduced pressure. The obtained was triturated with MeCN (100 mL) for half an hour. The solid was collected after filtration and concentration under reduced pressure. Compound 6-bromo-7-fluoro-2-methyl-10-oxo-4-oxa-1-azatricyclo [7.3.1.05,13]trideca-5(13),6,8,11-tetraene-11-carboxylic acid (19.5 g, 57.00 mmol, 99.12% yield) was obtained as pale yellow solid. ¹H NMR (DMSO-d₆, 400 MHz) δ 9.08 (s, 1H), 7.77 (d, J=8.8 Hz, 1H), 5.03˜5.01 (m, 2H), 4.75-4.72 (m, 1H), 4.54-4.51 (m, 1H), 1.47 (d, J=6.8 Hz, 3H).

### Preparation of 10-bromo-9-fluoro-3-methyl-5,6-dihydro-2H-[1,4]oxazino[2,3,4-ij]quinolin-7(3H)-one (Step 4 in Scheme 10)

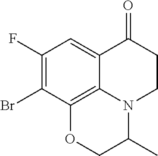 [SMILES: CC1COc2c(Br)c(F)cc3c2N1CCC3=O]

To a solution of 6-bromo-7-fluoro-2-methyl-10-oxo-4-oxa-1-azatricyclo[7.3.1.05,13]trideca-5(13),6,8,11-tetraene-11-carboxylic acid (13 g, 38.00 mmol, 1 eq) in EtOH (200 mL) was added NaBH₄ (21.2 g, 560.37 mmol, 14.75 eq) in portions at 0° C. during 3 hours under N₂. The mixture was stirred at 20° C. for 2 hours under N₂ atmosphere. Then TsOH·H₂O (722.81 mg, 3.80 mmol, 0.1 eq) was added and the mixture was stirred at 85° C. for 10 hours under N₂ atmosphere. LCMS and TLC (petroleum ether:EtOAc=2:1, R_{f}=0.50) showed ˜50% 6-bromo-7-fluoro-2-methyl-10-oxo-4-oxa-1-azatricyclo[7.3.1.05,13]trideca-5(13),6,8,11-tetraene-11-carboxylic acid was remained, ˜30% 6-bromo-7-fluoro-2-methyl-4-oxa-1-azatricyclo[7.3.1.05,13]trideca-5(13),6,8-trien-10-one was formed. The reaction mixture was concentrated under reduced pressure. The residue was quenched with water (300 mL) slowly at 0° C. and extracted with EtOAc (100 mL×3). The combined organic layer was washed with brine (50 mL×1), dried over Na₂SO₄, filtered and concentrated under reduced pressure. The three batches were workup and purified together. The residue was purified by flash silica gel chromatography (Biotage®; 220 g SepaFlash® Silica Flash Column, Eluent of 0˜69% gradient ethyl acetate/petroleum ether @150 mL/min.). The eluent was removed under reduced pressure. Compound 6-bromo-7-fluoro-2-methyl-4-oxa-1-azatricyclo[7.3.1.05,13]trideca-5(13),6,8-trien-10-one (22 g, 73.30 mmol, 64.30% yield) was obtained as yellow solid. ¹H NMR (DMSO-d₆, 400 MHz) δ 7.11 (d, J=7.2 Hz, 1H), 4.31˜4.27 (m, 2H), 3.58˜3.54 (m, 2H), 3.33˜3.32 (m, 1H), 2.73˜2.70 (m, 1H), 2.65˜2.64 (m, 1H), 1.19˜1.15 (m, 3H).

### Preparation of 10-bromo-9-fluoro-3-methyl-7-oxo-3,5,6,7-tetrahydro-2H-[1,4]oxazino[2,3,4-ij]quinoline-6-carbaldehyde (Step 5 in Scheme 10)

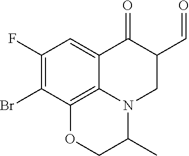 [SMILES: CC1COc2c(Br)c(F)cc3c2N1CC(C=O)C3=O]

To a mixture of NaOMe (15.44 g, 285.88 mmol, 3.9 eq) in DCM (400 mL) was added ethyl formate (21.18 g, 285.88 mmol, 22.99 mL, 3.9 eq) at 20° C. Then 6-bromo-7-fluoro-2-methyl-4-oxa-1-azatricyclo[7.3.1.05,13]trideca-5(13),6,8-trien-10-one (22 g, 73.30 mmol, 1 eq) was added in portions at 0° C. The mixture was stirred at 20° C. for 10 hours under N₂ atmosphere. LCMS and TLC (petroleum ether:EtOAc=2:1, Rf=0.5) showed the reaction was complete. The reaction mixture was poured into ice water (300 mL) slowly at 0° C. and made pH=6 with 0.5N HCl. The organic layer was separated and extracted with DCM (400 mL×2). The combined organic layer was washed with brine (200 mL×1), dried over Na₂SO₄, filtered and concentrated under reduced pressure. Compound 6-bromo-7-fluoro-2-methyl-10-oxo-4-oxa-1-azatricyclo [7.3.1.05,13]trideca-5(13),6,8-triene-11-carbaldehyde (23 g, 70.09 mmol, 95.62% yield) was obtained as brown solid. ¹H NMR (DMSO-d₆, 400 MHz) δ 11.76 (s, 1H), 7.84 (m, 1H), 7.14 (d, J=5.2 Hz, 1H), 4.26˜4.25 (m, 1H), 4.21˜4.17 (m, 2H), 3.90˜3.87 (m, 1H), 3.51˜3.50 (m, 1H), 1.21˜1.15 (m, 3H).

### Preparation of 10-bromo-9-fluoro-6-((((2-methoxypyridin-4-yl)methyl)((S)-1-(6-nitropyridin-3-yl)piperidin-3-yl)amino)methyl)-3-methyl-2H-[1,4]oxazino[2,3,4-ij]quinolin-7(3H)-one (Step 6 in Scheme 10)

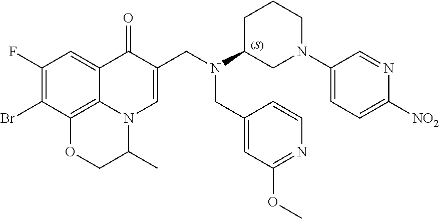

A solution of 6-bromo-7-fluoro-2-methyl-10-oxo-4-oxa-1-azatricyclo[7.3.1.05,13]trideca-5(13),6,8-triene-11-carbaldehyde (6.21 g, 18.93 mmol, 1.3 eq), (3S)—N-[(2-methoxy-4-pyridyl)methyl]-1-(6-nitro-3-pyridyl)piperidin-3-amine (5 g, 14.56 mmol, 1 eq) and AcOH (1.31 g, 21.84 mmol, 1.25 mL, 1.5 eq) in DCE (150 mL) was stirred at 95° C. for 3 hours. Then the mixture was cooled to 0° C. and NaBH(OAc)₃ (4.01 g, 18.93 mmol, 1.3 eq) was added in portions at 0° C. The reaction mixture was stirred at 20° C. for 10 hours. LCMS, HPLC and TLC (EtOAc:MeOH=10:1, R_{f}=0.10) showed (3S)—N-[(2-methoxy-4-pyridyl)methyl]-1-(6-nitro-3-pyridyl)piperidin-3-amine was remained, ˜11% of 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8-trien-10-one and ˜10% of 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8,11-tetraen-10-one were detected. The mixture was quenched with ice water (50 mL) at 0° C. and made pH=8 with sat. NaHCO₃. The mixture was extracted with DCM and i-PrOH (v:v=3:1, 50 mL×2). The combined organic layer was washed with brine (30 mL×1), dried over Na₂SO₄, filtered and concentrated under reduced pressure. The residue was purified by prep-HPLC (column: Xtimate C18 10 μm 250 mm*80 mm; mobile phase: [water (NH₄HCO₃)-MeCN]; B %: 45%-85%, 34 mins). The eluent was removed under freeze drying. The obtained was further purified by flash silica gel chromatography (Biotage®; 25 g SepaFlash® Silica Flash Column, Eluent of 0˜100% gradient ethyl acetate/petroleum ether @70 mL/min.). The eluent was removed under reduced pressure. Compound 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8-trien-10-one (2.5 g, 3.81 mmol, 26.19% yield) was obtained as orange solid. Compound 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8,11-tetraen-10-one (0.45 g, 688.60 umol, 4.73% yield) was obtained as orange solid. Also 2 g of (3S)—N-[(2-methoxy-4-pyridyl)methyl]-1-(6-nitro-3-pyridyl)piperidin-3-amine was recovered as yellow solid. 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8,11-tetraen-10-one (0.45 g, 688.60 umol, 4.73% yield) was obtained as orange solid. ¹H NMR (DMSO-d₆, 400 MHz) δ 8.26-8.25 (m, 1H), 8.11˜8.08 (m, 2H), 8.01˜7.99 (m, 1H), 7.51˜7.48 (m, 2H), 7.03˜7.02 (m, 1H), 6.87 (d, J=10.0 Hz, 1H), 4.61˜4.21 (m, 5H), 3.85˜3.83 (m, 1H), 3.76 (s, 3H), 3.73˜3.57 (m, 3H), 3.31˜3.17 (m, 1H), 2.98˜2.96 (m, 1H), 2.68˜2.67 (m, 1H), 1.77˜1.75 (m, 1H), 1.74˜1.73 (m, 1H), 1.43˜1.37 (m, 1H), 1.34˜1.16 (m, 4H).

### Preparation of 6-((((S)-1-(6-aminopyridin-3-yl)piperidin-3-yl)((2-methoxypyridin-4-yl)methyl)amino)methyl)-10-bromo-9-fluoro-3-methyl-2H-[1,4]oxazino[2,3,4-ij]quinolin-7(3H)-one (Step 7 in Scheme 10)

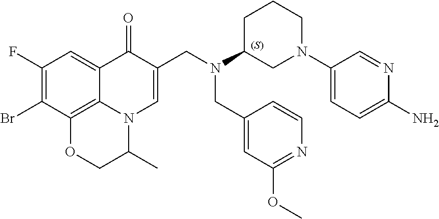

A mixture of 6-bromo-7-fluoro-11-[[(2-methoxy-4-pyridyl)methyl-[(3S)-1-(6-nitro-3-pyridyl)-3-piperidyl]amino]methyl]-2-methyl-4-oxa-1-azatricyclo[7.3.1.05,13]trideca-5(13),6,8,11-tetraen-10-one (0.65 g, 994.65 umol, 1 eq) and PtO₂ (53.14 mg, 198.93 umol, 85% purity, 0.2 eq) in DMF (70 mL) was stirred at 20° C. for an hour under H₂ (14.6 psi). Then the mixture was stirred at 20° C. for 1.5 hours under H₂ (14.6 psi). LCMS, HPLC and TLC (EtOAc:MeOH=5:1, R_{f}=0.1) showed the reaction was complete. The mixture was filtered through a pad of the Celie. The filtrate was concentrated under reduced pressure. The residue was purified by flash silica gel chromatography (Biotage®; 12 g SepaFlash® Silica Flash Column, Eluent of 0-18% gradient methanol/ethyl acetate @40 mL/min.). The eluent was removed under reduced pressure. Compound 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.05,13]trideca-5(13),6,8,11-tetraen-10-one (0.51 g, 817.94 umol, 82.23% yield) was obtained as yellow solid. ¹H NMR (DMSO-d₆, 400 MHz) δ 8.08 (d, J=7.2 Hz, 1H), 7.99 (d, J=5.6 Hz, 1H), 7.61 (d, J=3.2 Hz, 1H), 7.51 (dd, J=2.4 Hz, 9.2 Hz, 1H), 7.14-7.13 (m, 1H), 7.01˜6.99 (m, 1H), 6.82˜6.81 (m, 1H), 6.39˜6.36 (m, 1H), 5.38 (s, 2H), 4.71˜4.68 (m, 1H), 4.61˜4.59 (m, 1H), 4.44˜4.41 (m, 1H), 3.76 (s, 3H), 3.75˜3.59 (m, 5H), 3.32˜3.19 (m, 1H), 2.70˜2.67 (m, 1H), 2.61˜2.56 (m, 1H), 2.50˜2.41 (m, 1H), 1.99˜1.91 (m, 1H), 1.75˜1.74 (m, 1H), 1.49˜1.44 (m, 2H), 1.35˜1.32 (m, 3H). LCMS: 92.04% (220 nm), 93.43% (254 nm). MS (ESI): mass calcd. For C₃₀H₃₂BrFN₆O₃ 622.17 m/z found 623.3 [M+H]⁺.

### Preparation of Compounds in Scheme 10 (Step 8 in Scheme 10)

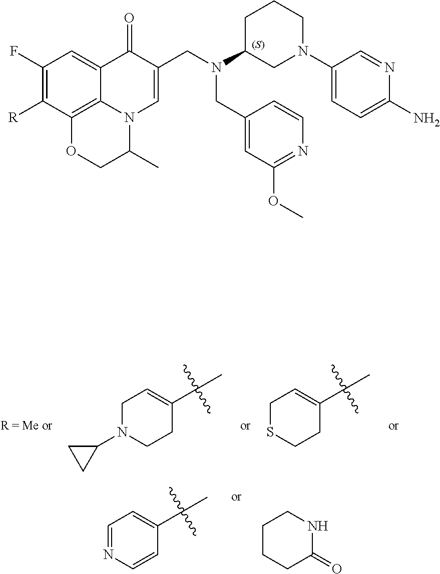

Method A: To a mixture of 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.05,13]trideca-5(13),6,8,11-tetraen-10-one (80.19 umol, 1 eq) in DMF (10 mL/mmol˜13 mL/mmol) and H₂O (1 mL/mmol˜1.3 mL/mmol) was added different boronic acid or boronic ester (120.28 umol˜240.57 umol, 1.5 eq˜3 eq), Pd(PPh₃)₄(8.02 umol, 0.1 eq) and NaHCO₃ (320.76 umol, 4 eq). The reaction mixture was degassed and purged with N₂ for 3 times. Then the mixture was stirred at 130° C. for 20 minutes to half an hour under N₂ atmosphere under microwave. The reaction mixture was cooled to room temperature and filtered through a pad of the Celite. The filtrate was concentrated under reduced pressure. The residue was purified by prep-HPLC (column: Phenomenex C18 75*30 mm*3 um or Waters Xbridge Prep OBD C18 150*40 mm*10 um or Waters Xbridge BEH C18 100*30 mm*10 um; mobile phase: [water (NH₄HCO₃)-MeCN]; B %: 20%-65%, 8 mins). The eluent was removed under freeze drying to give the desired product.
Method B: A mixture of different boronic acid or boronic ester (48.11 umol˜96.22 umol, 1.5 eq 3.0 eq), 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo [7.3.1.0^{5,13}]trideca-5(13),6,8,11-tetraen-10-one (32.08 umol, 1 eq), Pd(dppf)Cl₂ (1.60 umol˜3.20 umol, 0.05 eq˜0.1 eq), Na₂CO₃ (96.23 umol, 3 eq) or K₂CO₃ (96.23 umol, 3 eq) in dioxane (3 mL/mmol˜10 mL/mmol) and H₂O (0.3 mL/mmol˜1.0 mL/mmol) was degassed and purged with N₂ for 3 times and then the mixture was stirred at 80° C.˜90° C. for 3 hours˜4 hours under N₂ atmosphere. LC-MS and HPLC showed ˜15% of 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.0^{5,13}]trideca-5(13),6,8,11-tetraen-10-one (20 mg, 32.08 umol, 1 eq) was remained and ˜32% of desired product was detected. The reaction mixture was concentrated under reduced pressure. The residue was purified by prep-HPLC (column: Phenomenex C18 75*30 mm*3 um or Waters Xbridge Prep OBD C18 150*40 mm*10 um or Waters Xbridge BEH C18 100*30 mm*10 um; mobile phase: [water (NH₄HCO₃)-MeCN]; B %: 20%-65%, 8 mins).
Method C: To a solution of 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.05,13]trideca-5(13),6,8,11-tetraen-10-one (320.76 umol, 1 eq) in toluene (10 mL) was added Pd(OAc)₂ (16.04 umol, 0.05 eq), Xantphos (32.08 umol, 0.1 eq) and t-BuONa (641.52 umol, 2 eq). The mixture was stirred at 120° C. for 2 hours. The reaction mixture was filtered through a pad of the Celite and the filter cake was washed with MeOH (10 mL×3). The filtrate was concentrated under reduced pressure. The residue was purified by prep-HPLC (column: Waters Xbridge Prep OBD C 18 150*40 mm*10 um; mobile phase:[water (NH₄HCO₃)-MeCN]; B %: 25%-45%, 8 mins). The solvent was removed under freeze drying to give the desired product.

### Compound 56

### Preparation of 6-((((S)-1-(6-aminopyridin-3-yl)piperidin-3-yl)((2-methoxypyridin-4-yl)methyl)amino)methyl)-9-fluoro-3-methyl-10-(pyridin-4-yl)-2H-[1,4]oxazino[2,3,4-ij]quinolin-7(3H)-one (Step 8 in Scheme 10)

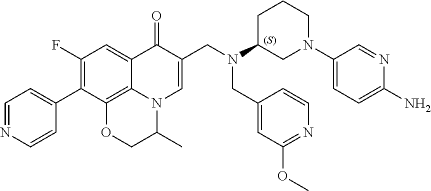

To a mixture of 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.05,13]trideca-5(13),6,8,11-tetraen-10-one (50 mg, 80.19 umol, 1 eq) in DMF (1 mL) and H₂O (0.1 mL) was added 4-pyridylboronic acid (29.57 mg, 240.57 umol, 3 eq), Pd(PPh₃)₄(9.27 mg, 8.02 umol, 0.1 eq) and NaHCO₃ (26.95 mg, 320.76 umol, 12.48 uL, 4 eq). The reaction mixture was degassed and purged with N₂ for 3 times. Then the mixture was stirred at 130° C. for half an hour under N₂ atmosphere under microwave. LCMS and HPLC showed the reaction was complete. The reaction mixture was cooled to room temperature and filtered through a pad of the Celite. The filtrate was concentrated under reduced pressure. The residue was purified by prep-HPLC (column: Waters Xbridge Prep OBD C18 150*40 mm*10 um; mobile phase: [water (NH₄HCO₃)-MeCN]; B %: 25%-50%, 8 mins). The eluent was removed under freeze drying. Compound 11-[[[(3S)-1-(6-amino-3-pyridyl)-3-piperidyl]-[(2-methoxy-4-pyridyl)methyl]amino]methyl]-7-fluoro-2-methyl-6-(4-pyridyl)-4-oxa-1-azatricyclo[7.3.1.05,13]trideca-5(13),6,8,11-tetraen-10-one (16.2 mg, 25.89 umol, 32.29% yield, 99.36% purity) was obtained as white solid. ¹H NMR (DMSO-d₆, 400 MHz) δ 8.71 (d, J=6.0 Hz, 2H), 8.09 (d, J=8.0 Hz, 1H), 8.01 (d, J=5.2 Hz, 1H), 7.61 (d, J=2.8 Hz, 1H), 7.52˜7.50 (m, 3H), 7.16-7.14 (m, 1H), 7.02˜7.00 (m, 1H), 6.83 (s, 1H), 6.39-6.36 (m, 1H), 5.37 (s, 2H), 4.68˜4.66 (m, 1H), 4.44˜4.42 (m, 1H), 4.36-4.33 (m, 1H), 3.77 (s, 3H), 3.74-3.36 (m, 5H), 3.24-3.19 (m, 1H), 2.77-2.67 (m, 1H), 2.63˜2.60 (m, 1H), 2.45˜2.42 (m, 1H), 1.99˜1.91 (m, 1H), 1.79˜1.74 (m, 1H), 1.48˜1.45 (m, 2H), 1.35˜1.32 (m, 3H). LCMS: 99.36% (220 nm), 99.57% (254 nm). MS (ESI): mass calcd. For C₃₅H₃₆FN₇O₃ 621.29 m/z found 622.4 [M+H]⁺.

### Compound 57

### 6-((((S)-1-(6-aminopyridin-3-yl)piperidin-3-yl)((2-methoxypyridin-4-yl)methyl)amino)methyl)-9-fluoro-3,10-dimethyl-2H-[1,4]oxazino[2,3,4-ij]quinolin-7(3H)-one was Prepared According to the Procedure Described Herein for Step 8 in Scheme 10

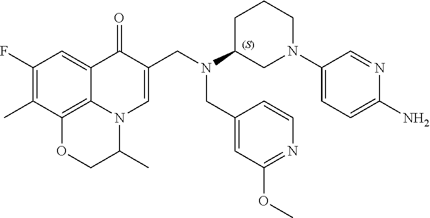

Method A: 11-[[[(3S)-1-(6-amino-3-pyridyl)-3-piperidyl]-[(2-methoxy-4-pyridyl)methyl]amino]methyl]-7-fluoro-2,6-dimethyl-4-oxa-1-azatricyclo[7.3.1.0^{5,13}]trideca-5(13),6,8,11-tetraen-10-one (5.9 mg, 10.16 umol, 12.67% yield, 96.21% purity) was obtained as white solid. ¹H NMR (DMSO-d₆, 400 MHz) δ 8.02˜7.98 (m, 2H), 7.60 (d, J=2.8 Hz, 1H), 7.35 (dd, J=2.4 Hz, 10.0 Hz, 1H), 7.15˜7.13 (m, 1H), 6.99˜6.98 (m, 1H), 6.81 (s, 1H), 6.37 (dd, J=3.6 Hz, 8.8 Hz, 1H), 5.37 (s, 2H), 4.63˜4.61 (m, 1H), 4.51˜4.48 (m, 1H), 4.32˜4.29 (m, 1H), 3.75 (s, 3H), 3.71˜3.65 (m, 2H), 3.63˜3.54 (m, 2H), 3.51˜3.48 (m, 1H), 3.23˜3.21 (m, 1H), 2.77˜2.73 (m, 1H), 2.60-2.55 (m, 1H), 2.43˜2.37 (m, 1H), 2.21 (d, J=1.6 Hz, 3H), 1.98-1.95 (m, 1H), 1.76-1.73 (m, 1H), 1.49-1.38 (m, 2H), 1.30 (dd, J=4.8 Hz, 6.8 Hz, 3H). LCMS: 96.21% (220 nm), 98.51% (254 nm). MS (ESI): mass calcd. For C₃₁H₃₅FN₆O₃ 558.28 m/z found 559.4 [M+H]⁺.

### Compound 58

### 6-((((S)-1-(6-aminopyridin-3-yl)piperidin-3-yl)((2-methoxypyridin-4-yl)methyl)amino)methyl)-10-(3,6-dihydro-2H-thiopyran-4-yl)-9-fluoro-3-methyl-2H-[1,4]oxazino[2,3,4-ij]quinolin-7(3H)-one was Prepared According to the Procedure Described Herein for Step 8 in Scheme 10

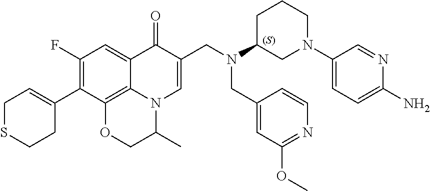

Method A: 11-[[[(3S)-1-(6-amino-3-pyridyl)-3-piperidyl]-[(2-methoxy-4-pyridyl)methyl]amino]methyl]-6-(3,6-dihydro-2H-thiopyran-4-yl)-7-fluoro-2-methyl-4-oxa-1-azatricyclo[7.3.1.0^{5,13}]trideca-5(13),6,8,11-tetraen-10-one (2 mg, 3.08 umol, 35.61% yield, 98.94% purity) was obtained as off-white solid. ¹H NMR (DMSO-d₆, 400 MHz) δ 8.04 (d, J=2.8 Hz, 1H), 8.00 (d, J=2.8 Hz, 1H), 7.61 (d, J=2.8 Hz, 1H), 7.38 (dd, J=2.4 Hz, 10.0 Hz, 1H), 7.16-7.12 (m, 1H), 7.00-6.99 (m, 1H), 6.81 (s, 1H), 6.37 (dd, J=3.6 Hz, 8.8 Hz, 1H), 5.92 (s, 1H), 5.36 (d, J=2.8 Hz, 2H), 4.63˜4.60 (m, 1H), 4.47-4.44 (m, 1H), 4.30˜4.27 (m, 1H), 3.75 (s, 3H), 3.72˜3.66 (m, 2H), 3.63˜3.57 (m, 2H), 3.51˜3.48 (m, 1H), 3.33˜3.30 (m, 2H), 3.24˜3.21 (m, 1H), 2.82 (t, J=5.6 Hz, 2H), 2.79-2.72 (m, 1H), 2.61˜2.56 (m, 1H), 2.46-2.44 (m, 2H), 2.41˜2.37 (m, 1H), 2.01˜1.93 (m, 1H), 1.77˜1.74 (m, 1H), 1.53˜1.38 (m, 2H), 1.31 (dd, J=4.8 Hz, 6.4 Hz, 3H). LCMS: 98.94% (220 nm), 98.22% (254 nm). MS (ESI): mass calcd. For C₃₅H₃₉FN₆O₃S 642.28 m/z found 643.4 [M+H]⁺.

### Compound 59

### 6-((((S)-1-(6-aminopyridin-3-yl)piperidin-3-yl)((2-methoxypyridin-4-yl)methyl)amino)methyl)-10-(1-cyclopropyl-1,2,3,6-tetrahydropyridin-4-yl)-9-fluoro-3-methyl-2H-[1,4]oxazino[2,3,4-ij]quinolin-7(3H)-one was Prepared According to the Procedure Described Herein for Step 8 in Scheme 10

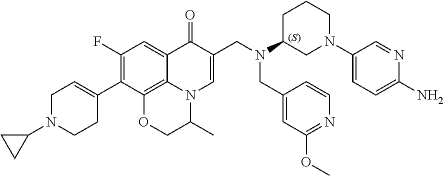

Method B: 11-[[[(3S)-1-(6-amino-3-pyridyl)-3-piperidyl]-[(2-methoxy-4-pyridyl)methyl]amino]methyl]-6-(1-cyclopropyl-3,6-dihydro-2H-pyridin-4-yl)-7-fluoro-2-methyl-4-oxa-1-azatricyclo [7.3.1.0^{5,13}]trideca-5(13),6,8,11-tetraen-10-one (2.4 mg, 3.50 umol, 10.90% yield, 97% purity) was obtained as pale yellow solid. ¹H NMR (DMSO-d₆, 400 MHz) δ 8.05-8.00 (m, 2H), 7.62 (s, 1H), 7.38 (dd, J=2.4 Hz, 10.0 Hz, 1H), 7.16-7.14 (m, 1H), 7.01˜7.00 (s, 1H), 6.82 (s, 1H), 6.39-6.37 (m, 1H), 5.75 (s, 1H), 5.38-5.34 (m, 2H), 4.63˜4.61 (m, 1H), 4.47 (d, J=10.8 Hz, 1H), 4.29 (d, J=10.0 Hz, 1H), 3.77 (s, 3H), 3.72˜3.49 (m, 5H), 3.23˜3.22 (m, 3H), 2.81 (t, J=5.2 Hz, 2H), 2.76-2.73 (m, 1H), 2.60-2.59 (m, 1H), 2.42˜2.41 (m, 1H), 2.32˜2.31 (m, 2H), 1.98-1.96 (m, 1H), 1.77-1.75 (m, 2H), 1.50-1.42 (m, 2H), 1.31 (t, J=6.0 Hz, 3H), 0.47-0.46 (m, 2H), 0.40-0.39 (m, 2H). LCMS: 97.00% (220 nm), 98.43% (254 nm). MS (ESI): mass calcd. For C₃₈H₄₄FN₇O₃ 665.35 m/z found 666.5 [M+H]⁺.

### Compound 60

### 6-((((S)-1-(6-aminopyridin-3-yl)piperidin-3-yl)((2-methoxypyridin-4-yl)methyl)amino)methyl)-9-fluoro-3-methyl-10-(2-oxopiperidin-1-yl)-2H-[1,4]oxazino[2,3,4-ij]quinolin-7(3H)-one was Prepared According to the Procedure Described Herein for Step 8 in Scheme 10

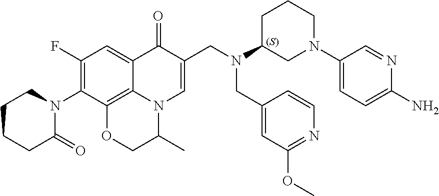

Method C: Compound 11-[[[(3S)-1-(6-amino-3-pyridyl)-3-piperidyl]-[(2-methoxy-4-pyridyl)methyl]amino]methyl]-7-fluoro-2-methyl-6-[(1S)-2-oxo-1-piperidyl]-4-oxa-1-azatricyclo[7.3.1.05,13]trideca-5(13),6,8,11-tetraen-10-one (23.1 mg, 35.83 umol, 22.34% yield, 99.53% purity) was obtained as off white solid. ¹H NMR (DMSO-d₆, 400 MHz) δ 8.08 (d, J=8.0 Hz, 1H), 8.01 (d, J=5.2 Hz, 1H), 7.61 (d, J=2.4 Hz, 1H), 7.45 (d, J=10.4 Hz, 1H), 7.14-7.11 (m, 1H), 7.01˜6.99 (m, 1H), 6.83 (d, J=3.2 Hz, 1H), 6.36 (t, J=9.2 Hz, 1H), 5.37-5.35 (m, 2H), 4.67-4.66 (m, 1H), 4.52˜4.50 (m, 1H), 4.38˜4.35 (m, 1H), 3.77 (s, 3H), 3.72˜3.67 (m, 2H), 3.64˜3.56 (m, 3H), 3.51˜3.48 (m, 1H), 3.43˜3.37 (m, 1H), 3.25˜3.21 (m, 1H), 2.78˜2.75 (m, 1H), 2.62˜2.59 (m, 1H), 2.45˜2.42 (m, 3H), 1.98˜1.96 (m, 1H), 1.88˜1.87 (m, 4H), 1.78˜1.74 (m, 1H), 1.46-1.44 (m, 2H), 1.29 (d, J=5.6 Hz, 3H). LCMS: 99.53% (220 nm), 98.64% (254 nm). MS (ESI): mass calcd. For C₃₅H₄₀FN₇O₄ 641.31 m/z found 642.5 [M+H]⁺.

### Compound 61

### 6-((((S)-1-(6-aminopyridin-3-yl)piperidin-3-yl)((2-methoxypyridin-4-yl)methyl)amino)methyl)-9-fluoro-3-methyl-10-(2-oxopiperidin-1-yl)-2H-[1,4]oxazino[2,3,4-ij]quinolin-7(3H)-one was Prepared According to the Procedure Described Herein for Step 8 in Scheme 10

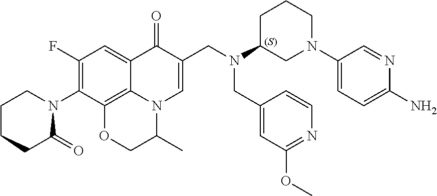

Method C: Compound 11-[[[(3S)-1-(6-amino-3-pyridyl)-3-piperidyl]-[(2-methoxy-4-pyridyl) methyl]amino]methyl]-7-fluoro-2-methyl-6-[(1S)-2-oxo-1-piperidyl]-4-oxa-1-azatricyclo[7.3.1.05,13]trideca-5(13),6,8,11-tetraen-10-one (23 mg, 35.56 umol, 22.17% yield, 99.22% purity) was obtained as pale yellow solid. ¹H NMR (DMSO-d₆, 400 MHz) δ 8.09 (s, 1H), 8.01 (d, J=4.8 Hz, 1H), 7.61 (s, 1H), 7.46 (d, J=10.0 Hz, 1H), 7.15 (dd, J=2.4 Hz, 8.4 Hz, 1H), 7.01˜7.00 (m, 1H), 6.83 (s, 1H), 6.37 (d, J=8.4 Hz, 1H), 5.37 (s, 2H), 4.66-4.64 (m, 1H), 4.51˜4.48 (m, 1H), 4.29-4.27 (m, 1H), 3.77 (s, 3H), 3.72˜3.70 (m, 2H), 3.66-3.62 (m, 1H), 3.60-3.56 (m, 2H), 3.56-3.48 (m, 1H), 3.43˜3.40 (m, 2H), 3.24-3.21 (m, 1H), 2.77-2.71 (m, 1H), 2.57˜2.56 (m, 1H), 2.45˜2.42 (m, 3H), 1.98˜1.96 (m, 1H), 1.88˜1.86 (m, 4H), 1.76˜1.74 (m, 1H), 1.50-1.39 (m, 2H), 1.33˜1.29 (m, 3H). LCMS: 99.22% (220 nm), 98.36% (254 nm). MS (ESI): mass calcd. For C₃₅H₄₀FN₇O₄ 641.31 m/z found 642.5 [M+H]⁺.

### Compound 62

### Preparation of 6-((((S)-1-(6-aminopyridin-3-yl)piperidin-3-yl)((2-methoxypyridin-4-yl)methyl)amino)methyl)-10-(benzyloxy)-9-fluoro-3-methyl-2H-[1,4]oxazino[2,3,4-ij]quinolin-7(3H)-one (Step 9 in Scheme 10)

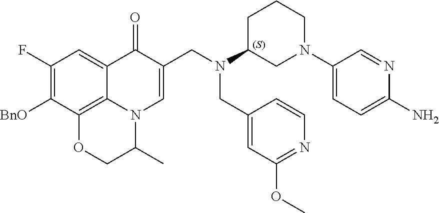

To a solution of BnOH (76.88 mg, 710.97 umol, 73.93 uL, 2 eq) in NMP (1 mL) was added NaH (28.44 mg, 710.97 umol, 60% purity, 2 eq) at 0° C. The mixture was stirred at 0° C. for half an hour. Then to the mixture was added a solution of 11-[[[(3S)-1-(6-amino-3-pyridyl)-3-piperidyl]-[(2-methoxy-4-pyridyl)methyl]amino]methyl]-6,7-difluoro-2-methyl-4-oxa-1-azatricyclo [7.3.1.0^{5,13}]trideca-5(13),6,8,11-tetraen-10-one (0.2 g, 355.49 umol, 1 eq) in NMP (2 mL) slowly at 0° C. The reaction mixture was stirred at 0° C. for an hour. LCMS and HPLC indicated the reaction was complete. The reaction mixture was quenched by addition ice water (5 mL) slowly at 0° C. and then extracted with EtOAc (10 mL×2). The combined organic layers were washed with brine (5 mL×2), dried over anhydrous Na₂SO₄, filtered and concentrated under reduced pressure. The residue was purified by prep-HPLC (column: Phenomenex C18 75*30 mm*3 um; mobile phase: [water (NH₄HCO₃)-MeCN]; B %: 35%-65%, 8 mins). The eluent was dried over lyophilization. Compound 11-[[[(3S)-1-(6-amino-3-pyridyl)-3-piperidyl]-[(2-methoxy-4-pyridyl)methyl]amino]methyl]-6-benzyloxy-7-fluoro-2-methyl-4-oxa-1-azatricyclo[7.3.1.0^{5,13}]trideca-5(13),6,8,11-tetraen-10-one (3.8 mg, 5.62 umol, 1.58% yield, 96.25% purity) was obtained as pale yellow solid. ¹H NMR (DMSO-d₆, 400 MHz) δ 8.01˜7.98 (m, 2H), 7.61 (d, J=2.8 Hz, 1H), 7.44-7.42 (m, 2H), 7.40-7.30 (m, 4H), 7.16-7.12 (m, 1H), 7.00-6.99 (m, 1H), 6.82 (s, 1H), 6.37 (dd, J=2.8 Hz, 8.8 Hz, 1H), 5.37 (d, J=3.2 Hz, 2H), 5.21 (s, 2H), 4.64-4.62 (m, 1H), 4.51˜4.48 (m, 1H), 4.28˜4.25 (m, 1H), 3.89˜3.79 (m, 1H), 3.76 (s, 3H), 3.71˜3.70 (m, 1H), 3.66-3.57 (m, 2H), 3.54-3.48 (m, 1H), 3.24-3.21 (m, 1H), 2.77-2.71 (m, 1H), 2.60-2.55 (m, 1H), 2.43˜2.37 (m, 1H), 1.98˜1.95 (m, 1H), 1.77˜1.74 (m, 1H), 1.53˜1.38 (m, 2H), 1.31˜1.29 (m, 3H). LCMS: 96.25% (220 nm), 96.50% (254 nm). MS (ESI): mass calcd. For C₃₇H₃₉FN₆O₄ 650.30 m/z found 651.5 [M+H]⁺.

### Compound 63

### Preparation of 6-((((S)-1-(6-aminopyridin-3-yl)piperidin-3-yl)((2-methoxypyridin-4-yl)methyl)amino)methyl)-9-fluoro-10-hydroxy-3-methyl-2H-[1,4]oxazino[2,3,4-ij]quinolin-7(3H)-one (Step 10 in Scheme 10)

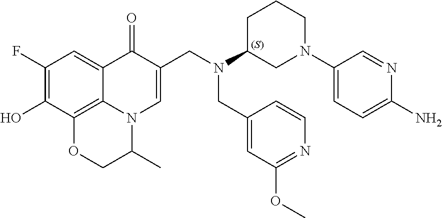

To a solution of 11-[[[(3S)-1-(6-amino-3-pyridyl)-3-piperidyl]-[(2-methoxy-4-pyridyl)methyl]amino]methyl]-6-benzyloxy-7-fluoro-2-methyl-4-oxa-1-azatricyclo[7.3.1.0^{5,13}]trideca-5(13),6,8,11-tetraen-10-one (80 mg, 122.94 umol, 1 eq) in MeOH (10 mL) was added Pd/C (10 mg, 10% purity). The suspension was degassed under vacuum and purged with H₂ several times. The reaction mixture was stirred under H₂ (15 psi) at 20° C. for 3 hours. LCMS and HPLC indicated 11-[[[(3S)-1-(6-amino-3-pyridyl)-3-piperidyl]-[(2-methoxy-4-pyridyl)methyl]amino]methyl]-6-benzyloxy-7-fluoro-2-methyl-4-oxa-1-azatricyclo[7.3.1.0^{5,13}]trideca-5(13),6,8,11-tetraen-10-one was nearly consumed and major one peak with desired mass was detected. The reaction mixture was filtered through a pad of the Celite and the filter cake was washed with MeOH (50 mL). The combined filtrate was concentrated under reduced pressure. The residue was purified by prep-HPLC (column: Phenomenex C18 75*30 mm*3 um; mobile phase: [water (NH₄HCO₃)-MeCN]; B %: 10%-40%, 8 mins). The eluent was dried over lyophilization. Compound 11-[[[(3S)-1-(6-amino-3-pyridyl)-3-piperidyl]-[(2-methoxy-4-pyridyl)methyl]amino]methyl]-7-fluoro-6-hydroxy-2-methyl-4-oxa-1-azatricyclo[7.3.1.0^{5,13}]trideca-5(13),6,8,11-tetraen-10-one (8.5 mg, 15.05 umol, 12.24% yield, 99.25% purity) was obtained as white solid. ¹H NMR (DMSO-d₆, 400 MHz) δ 10.30-10.22 (m, 1H), 7.99 (d, J=2.4 Hz, 1H), 7.94 (d, J=2.4 Hz, 1H), 7.60 (d, J=2.8 Hz, 1H), 7.36 (dd, J=3.6 Hz, 8.8 Hz, 1H), 7.16-7.12 (m, 1H), 7.00 (d, J=4.8 Hz, 1H), 6.82 (s, 1H), 6.37 (dd, J=3.6 Hz, 8.8 Hz, 1H), 5.36 (d, J=2.8 Hz, 2H), 4.60˜4.58 (m, 1H), 4.44 (d, J=10.0 Hz, 1H), 4.25 (d, J=10.8 Hz, 1H), 3.79-3.75 (m, 4H), 3.71˜3.70 (m, 1H), 3.66-3.60 (m, 1H), 3.57˜3.48 (m, 1H), 3.53˜3.48 (m, 1H), 3.24˜3.22 (m, 1H), 2.77˜2.72 (m, 1H), 2.60˜2.55 (m, 1H), 2.43˜2.37 (m, 1H), 1.97˜1.95 (m, 1H), 1.77˜1.74 (m, 1H), 1.50˜1.38 (m, 2H), 1.31 (dd, J=4.8 Hz, 6.4 Hz, 3H). LCMS: 99.25% (220 nm), 99.76% (254 nm). MS (ESI): mass calcd. For C₃₀H₃₃FN₆O₄ 560.25 m/z found 561.4 [M+H]⁺.

### Compound 64

### Preparation of 5-(2-((tert-butyldimethylsilyl)oxy)ethyl)-2-(tributylstannyl)pyridine (Step 11 in Scheme 10)

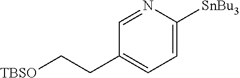

A mixture of tert-butyl-[2-(6-chloro-3-pyridyl)ethoxy]-dimethyl-silane (0.5 g, 1.84 mmol, 1 eq), trimethyl(trimethylstannyl)stannane (0.99 g, 3.02 mmol, 626.58 uL, 1.64 eq) and Pd(PPh₃)₄ (212.53 mg, 183.92 umol, 0.1 eq) in dioxane (5 mL) was degassed and purged with N₂ for 3 times and then the mixture was stirred at 90° C. for 10 hours under N₂ atmosphere. LCMS showed ˜18% of tert-butyl-[2-(6-chloro-3-pyridyl)ethoxy]-dimethyl-silane was remained. Several new peaks were shown on LCMS and ˜31% of desired compound was detected. The reaction mixture was filtered through a pad of the Celite and the filter cake was washed with EtOAc (10 mL). To the filtrate was added water (40 mL) and extracted with EtOAc (10 mL×3). The combined organic layer was washed with brine (10 mL), dried over Na₂SO₄, filtered and concentrated under reduced pressure. The residue was purified by flash silica gel chromatography (Biotage®; 12 g SepaFlash® Silica Flash Column, Eluent of 0-10% petroleum ether/Ethyl acetate gradient @40 mL/min.). The eluent was removed under reduced pressure. Compound tert-butyl-dimethyl-[2-(6-trimethylstannyl-3-pyridyl)ethoxy]silane (0.4 g, crude) was obtained as yellow oil. ¹H NMR (DMSO-d₆, 400 MHz) δ 8.60-8.61 (m, 1H), 7.56-7.53 (m, 1H), 7.25-7.23 (m, 1H), 3.83˜3.80 (m, 2H), 2.83˜2.77 (m, 2H), 0.86 (s, 18H), -0.03 (t, J=6.4 Hz, 6H).

### Preparation of 6-((((S)-1-(6-aminopyridin-3-yl)piperidin-3-yl)((2-methoxypyridin-4-yl)methyl)amino)methyl)-10-(5-(2-((tert-butyldimethylsilyl)oxy)ethyl)pyridin-2-yl)-9-fluoro-3-methyl-2H-[1,4]oxazino[2,3,4-ij]quinolin-7(3H)-one (Step 12 in Scheme 10)

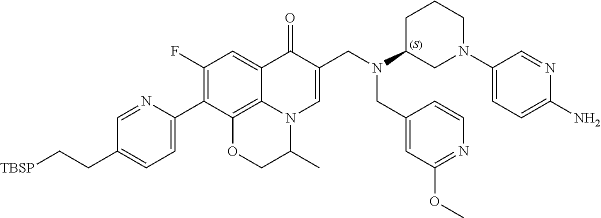

A mixture of tert-butyl-dimethyl-[2-(6-trimethylstannyl-3-pyridyl)ethoxy]silane (96.28 mg, 240.57 umol, 5 eq), 11-[[[(3S)-1-(6-amino-3-pyridyl)-3-piperidyl]-[(2-methoxy-4-pyridyl) methyl]amino]methyl]-6-bromo-7-fluoro-2-methyl-4-oxa-1-azatricyclo[7.3.1.0^{5,13}]trideca-5(13),6,8,11-tetraen-10-one (30 mg, 48.11 umol, 1 eq) and Pd(PPh₃)₄(1.67 mg, 1.44 umol, 0.03 eq) in dioxane (0.5 mL) was degassed and purged with N₂ for 3 times, Then the mixture was stirred at 90° C. for 12 hours under N₂ atmosphere. LC-MS and HPLC showed 11-[[[(3S)-1-(6-amino-3-pyridyl)-3-piperidyl]-[(2-methoxy-4-pyridyl)methyl]amino]methyl]-6-bromo-7-fluoro-2-methyl-4-oxa-1-azatricyclo[7.3.1.0^{5,13}]trideca-5(13),6,8,11-tetraen-10-one was remained. Several new peaks were shown on LCMS and one small peak with desired mass was detected. The mixture was concentrated under reduced pressure. The residue was purified by prep-HPLC (column: Waters Xbridge BEH C18 100*30 mm*10 um; mobile phase: [water (NH₄HCO₃)-MeCN]; B %: 60%-90%, 8 mins). The eluent was concentrated under reduced pressure. The residue was further purified by prep-TLC (SiO₂, EtOAc:MeOH=8:1). Compound 11-[[[(3S)-1-(6-amino-3-pyridyl)-3-piperidyl]-[(2-methoxy-4-pyridyl)methyl]amino]methyl]-6-[5-[2-[tert-butyl(dimethyl)silyl]oxyethyl]-2-pyridyl]-7-fluoro-2-methyl-4-oxa-1-azatricyclo[7.3.1.0^{5,13}]trideca-5(13),6,8,11-tetraen-10-one (8 mg, crude) was obtained as yellow solid. MS (ESI): mass calcd. For C₄₃H₅₄FN₇O₄Si 779.40 m/z found 780.1 [M+H]⁺.

### Preparation of 6-((((S)-1-(6-aminopyridin-3-yl)piperidin-3-yl)((2-methoxypyridin-4-yl)methyl)amino)methyl)-9-fluoro-10-(5-(2-hydroxyethyl)pyridin-2-yl)-3-methyl-2H-[1,4]oxazino[2,3,4-ij]quinolin-7(3H)-one (Step 13 in Scheme 10)

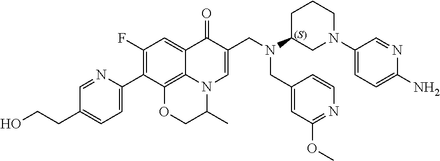

To a solution of 11-[[[(3S)-1-(6-amino-3-pyridyl)-3-piperidyl]-[(2-methoxy-4-pyridyl)methyl]amino]methyl]-6-[5-[2-[tert-butyl(dimethyl)silyl]oxyethyl]-2-pyridyl]-7-fluoro-2-methyl-4-oxa-1-azatricyclo[7.3.1.0^{5,13}]trideca-5(13),6,8,11-tetraen-10-one (8 mg, 10.26 umol, 1 eq) in MeOH (0.5 mL) was added HCl/MeOH (4 M, 1 mL). The mixture was stirred at 20° C. for an hour. TLC (Ethyl acetate:Methanol=0:1, R_{f}=0) showed the reaction was complete. The mixture was concentrated under reduced pressure. To the residue was added water (2 mL) and then adjusted pH=7 with sat. NaHCO₃. The mixture was extracted with EtOAc (7 mL×3). The combined organic layer was washed with brine (7 mL×1), dried over Na₂SO₄, filtered and concentrated under reduced pressure. The residue was purified by prep-TLC (SiO₂, EtOAc:MeOH=0:1). Compound 11-[[[(3S)-1-(6-amino-3-pyridyl)-3-piperidyl]-[(2-methoxy-4-pyridyl)methyl]amino]methyl]-7-fluoro-6-[5-(2-hydroxyethyl)-2-pyridyl]-2-methyl-4-oxa-1-azatricyclo[7.3.1.0^{5,13}]trideca-5(13),6,8,11-tetraen-10-one (2.1 mg, 2.97 umol, 28.95% yield, 94.13% purity) was obtained as white solid. ¹H NMR (DMSO-d₆, 400 MHz) δ 8.56-8.54 (m, 1H), 8.10-8.08 (m, 1H), 8.01 (d, J=5.2 Hz, 1H), 7.77 (dd, J=2.4, 8.0 Hz, 1H), 7.62˜7.61 (m, 1H), 7.49-7.45 (m, 2H), 7.17-7.14 (m, 1H), 7.02˜7.00 (m, 1H), 6.84-6.83 (m, 1H), 6.39-6.36 (m, 1H), 5.39-5.36 (m, 2H), 4.70˜4.64 (m, 1H), 4.40˜4.37 (m, 1H), 4.31˜4.28 (m, 1H), 3.77 (s, 3H), 3.74-3.51 (m, 6H), 3.36-3.35 (m, 2H), 3.26-3.21 (m, 1H), 2.80 (t, J=6.8 Hz, 2H), 2.75-2.72 (m, 1H), 2.64˜2.56 (m, 1H), 2.45˜2.41 (m, 1H), 2.01˜1.97 (m, 1H), 1.79˜1.75 (m, 1H), 1.51˜1.41 (m, 2H), 1.33˜1.31 (m, 3H). LCMS: 94.13% (220 nm), 99.21% (254 nm). MS (ESI): mass calcd. For C37H₄₀FN₇O₄ 665.31 m/z found 666.5 [M+H]⁺.

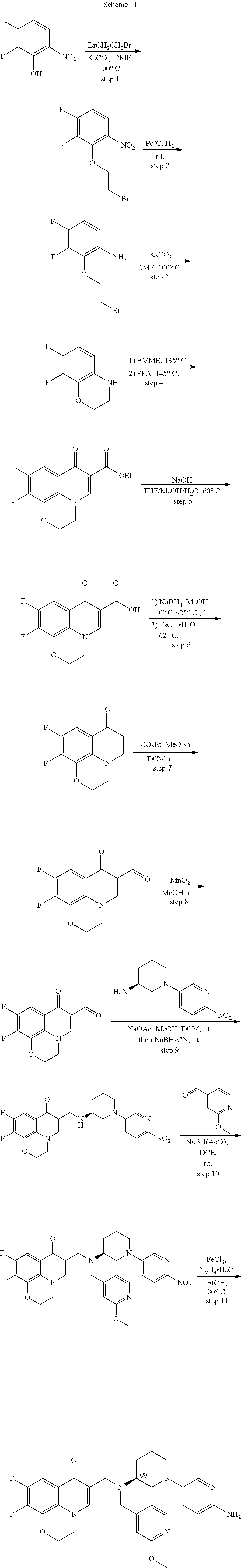

### General Procedures for Preparing Compounds in Scheme 11

### Preparation of 3-[(2-bromoethyl)oxy]-1,2-difluoro-4-nitrobenzene (Step 1 in Scheme 11)

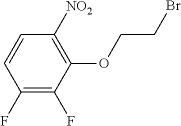 [SMILES: O=[N+]([O-])c1ccc(F)c(F)c1OCCBr]

A mixture of 2,3-difluoro-6-nitrophenol (5 g, 28.57 mmol) and K₂CO₃ (11.85 g, 85.71 mmol) in DMF (50 mL) was added BrCH₂CH₂Br (15 mL, 171.34 mmol). The reaction mixture was stirred at 100° C. for 3 hours. TLC (PE:EA=10:1) showed the reaction was complete. The reaction mixture was cooled to 25° C. and poured into H₂O. The mixture was extracted with AcOEt. The combined organic layer was washed with brine, dried over Na₂SO₄, filtered and concentrated. The residue was purified by column chromatography (SiO₂, PE:EA=25:1) to give 3-[(2-bromoethyl)oxy]-1,2-difluoro-4-nitrobenzene (3.90 g, 38.75%) as a yellow oil. ¹H NMR (DMSO-d₆, 400 MHz) δ: 7.88-7.95 (m, 1H), 7.40-7.53 (m, 1H), 4.58 (t, J=5.4 Hz, 2H), 3.79 (t, J=5.4 Hz, 2H).

### Preparation of 2-[(2-bromoethyl)oxy]-3,4-difluoroaniline (Step 2 in Scheme 11)

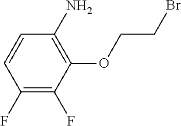 [SMILES: Nc1ccc(F)c(F)c1OCCBr]

To a solution of 3-[(2-bromoethyl)oxy]-1,2-difluoro-4-nitrobenzene (3.0 g, 10.68 mmol) in MeOH (15 mL) was added Pd/C (0.60 g) under hydrogen atmosphere. The reaction mixture was stirred at r.t. for 3 hours. LC-MS showed that 3-[(2-bromoethyl)oxy]-1,2-difluoro-4-nitrobenzene was consumed and one major peak with desired mass was detected. The reaction mixture was filtered. The filtrate was diluted with MeOH (30 mL). The organic layer was concentrated under vacuum. The residue was diluted with DCM (20 mL). The mixture washed with brine, dried over Na₂SO₄, filtered and concentrated. The crude product was purified by column chromatography (SiO₂, PE:EA=15:1) to give 2-[(2-bromoethyl)oxy]-3,4-difluoroaniline (1.87 g, 70%) as a brown oil. MS (ESI): mass calcd. For C₈H₈BrF₂NO 250.98 m/z found 251.9 [M+H]⁺.

### Preparation of 7,8-difluoro-3,4-dihydro-2H-benzo[b][1,4]oxazine (Step 3 in Scheme 11)

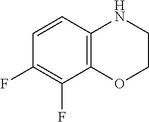 [SMILES: Fc1ccc2c(c1F)OCCN2]

To a solution of 2-[(2-bromoethyl)oxy]-3,4-difluoroaniline (500 mg, 1.98 mmol) in DMF (3 mL) was added potassium carbonate (822.42 mg, 5.95 mmol). The reaction mixture was stirred at 100° C. for 1 hour. LC-MS showed that 2-[(2-bromoethyl)oxy]-3,4-difluoroaniline was consumed and one major peak with desired mass was detected. The reaction mixture was cooled to 25° C. and poured into ice-water. The mixture was extracted with AcOEt. The combined organic layer was washed with brine, dried over Na₂SO₄, filtered and concentrated to give 7,8-difluoro-3,4-dihydro-2H-benzo[b][1,4]oxazine (270 mg, 79.4%) as a brown solid. MS (ESI): mass calcd. For C₈H₇F₂NO 171.15 m/z found 172.1 [M+H]⁺.

### Preparation of ethyl 9,10-difluoro-7-oxo-2,3-dihydro-7H-[1,4]oxazino[2,3,4-ij]quinoline-6-carboxylate (Step 4 in Scheme 11)

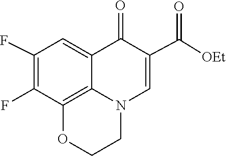 [SMILES: CCOC(=O)c1cn2c3c(c(F)c(F)cc3c1=O)OCC2]

A mixture of 7,8-difluoro-3,4-dihydro-2H-benzo[b][1,4]oxazine (364 mg, 2.13 mmol) and EMME (0.43 mL, 2.13 mmol) was stirred at 135° C. for 2 hours. PPE (2.0 g) was added to the mixture. The reaction mixture was stirred at 145° C. for 1.5 hours. LCMS showed the reaction was complete. The reaction mixture was cooled to 25° C. and poured into ice-water. The mixture was extracted with DCM. The combined organic layer was washed with brine, dried over Na₂SO₄, filtered and concentrated to give ethyl 9,10-difluoro-7-oxo-2,3-dihydro-7H-[1,4]oxazino[2,3,4-ij]quinoline-6-carboxylate (430 mg, 68.47%) as a yellow solid. MS (ESI): mass calcd. For C₁₄H₁₁F₂NO₄ 295.07 m/z found 296.0 [M+H]⁺.

### Preparation of 9,10-difluoro-7-oxo-2,3-dihydro[1,4]oxazino[2,3,4-ij]quinoline-6-carboxylic Acid (Step 5 in Scheme 11)

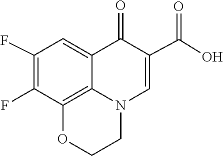 [SMILES: O=C(O)c1cn2c3c(c(F)c(F)cc3c1=O)OCC2]

To a solution of ethyl 9,10-difluoro-7-oxo-2,3-dihydro-7H-[1,4]oxazino[2,3,4-ij]quinoline-6-carboxylate (430 mg, 1.46 mmol) in THF/MeOH/H₂O (3 mL/0.4 mL/1.5 mL) was added NaOH (120 mg, 3 mmol). The reaction mixture was stirred at 60° C. for 30 min. LC-MS showed that ethyl 9,10-difluoro-7-oxo-2,3-dihydro-7H-[1,4]oxazino[2,3,4-ij]quinoline-6-carboxylate was consumed. The reaction mixture was cooled to 25° C. and poured into H₂O. The mixture was adjusted to pH=6 with 2N HCl. The mixture was filtered. The filter cake was dried to give 9,10-difluoro-7-oxo-2,3-dihydro[1,4]oxazino[2,3,4-ij]quinoline-6-carboxylic acid (267 mg, 68.6%) as a yellow solid. MS (ESI): mass calcd. For C₁₂H₇F₂NO₄ 267.03 m/z found 268.0 [M+H]⁺.

### Preparation of 9,10-difluoro-3,5,6,7-tetrahydro-2H-[1,4]oxazino[2,3,4-ij]quinolin-7-one (Step 6 in Scheme 11)

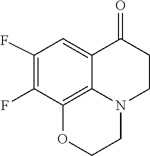 [SMILES: O=C1CCN2CCOc3c(F)c(F)cc1c32]

To a solution of 9,10-difluoro-7-oxo-2,3-dihydro[1,4]oxazino[2,3,4-ij]quinoline-6-carboxylic acid (217 mg, 0.81 mmol) in MeOH (1 mL) was added NaBH₄ (122.90 mg, 3.25 mmol) in portions at 0° C. under N₂. The reaction mixture was stirred at 25° C. for 1 hour. TsOH (3.09 mg, 0.02 mmol) was added to the reaction mixture. The reaction mixture was stirred at 62° C. for overnight. LC-MS showed that 9,10-difluoro-7-oxo-2,3-dihydro[1,4]oxazino[2,3,4-ij]quinoline-6-carboxylic acid was consumed. The reaction mixture was cooled to 25° C. and poured into ice-water. The mixture was adjusted to pH=7 with 2N HCl and extracted with AcOEt. The combined organic layer was washed with brine, dried over Na₂SO₄, filtered and concentrated. The crude was purified by column chromatography (SiO₂, PE:EA=3:1) to give 9,10-difluoro-3,5,6,7-tetrahydro-2H-[1,4]oxazino[2,3,4-ij]quinolin-7-one (128 mg, 69.9%) as a yellow solid. MS (ESI): mass calcd. For C₁₁H₉F₂NO₂ 225.06 m/z found 226.0 [M+H]⁺.

### Preparation of 9,10-difluoro-7-oxo-2,3,6,7-tetrahydro-5H-[1,4]oxazino[2,3,4-ij]quinoline-6-carbaldehyde (Step 7 in Scheme 11)

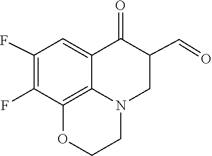 [SMILES: O=CC1CN2CCOc3c(F)c(F)cc(c32)C1=O]

A mixture of 9,10-difluoro-3,5,6,7-tetrahydro-2H-[1,4]oxazino[2,3,4-ij]quinolin-7-one (1.79 g, 7.96 mmol) and MeONa (2.6 mL, 30.23 mmol) in DCM (10 mL) was added HCO₂Et (168.43 mg, 31.82 mmol). The reaction mixture was stirred at 25° C. for 1 minute. LC-MS showed that 9,10-difluoro-3,5,6,7-tetrahydro-2H-[1,4]oxazino[2,3,4-ij]quinolin-7-one was consumed. The reaction mixture was poured into ice-water. The reaction mixture was adjusted to pH=6 with 2N HCl and extracted with AcOEt. The combined organic layer was washed with brine, dried over Na₂SO₄, filtered and concentrated to give 9,10-difluoro-7-oxo-2,3,6,7-tetrahydro-5H-[1,4]oxazino[2,3,4-ij]quinoline-6-carbaldehyde (1.965 g, 97.6%) as a yellow solid. MS (ESI): mass calcd. For C₁₂H₉F₂NO₃ 253.06 m/z found 254.0 [M+H]⁺.

### Preparation of 9,10-difluoro-7-oxo-2,3-dihydro-7H-[1,4]oxazino[2,3,4-ij]quinoline-6-carbaldehyde (Step 8 in Scheme 11)

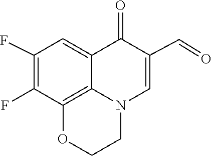 [SMILES: O=Cc1cn2c3c(c(F)c(F)cc3c1=O)OCC2]

To a solution of 9,10-difluoro-7-oxo-2,3,6,7-tetrahydro-5H-[1,4]oxazino[2,3,4-ij]quinoline-6-carbaldehyde (1.965 g, 7.77 mmol) in MeOH (10 mL) was added MnO₂ (1.012 g, 11.65 mmol). The reaction mixture was stirred at 25° C. for overnight. LC-MS showed that 9,10-difluoro-7-oxo-2,3,6,7-tetrahydro-5H-[1,4]oxazino[2,3,4-ij]quinoline-6-carbaldehyde was consumed. The reaction mixture was diluted with DCM/MeOH, filtered and concentrated. The crude was purified by column chromatography (SiO₂, DCM:MeOH=10:1) to give 9,10-difluoro-7-oxo-2,3-dihydro-7H-[1,4]oxazino[2,3,4-ij]quinoline-6-carbaldehyde (1.008 g, 51.7%) as a yellow solid. MS (ESI): mass calcd. For C₁₂H₇F₂NO₃ 251.04 m/z found 252.0 [M+H]⁺.

### Preparation of (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one (Step 9 in Scheme 11)

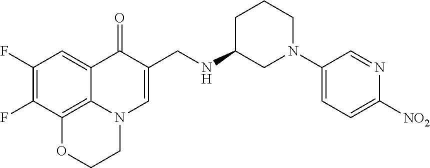 [SMILES: O=c1c(CN[C@H]2CCCN(c3ccc([N+](=O)[O-])nc3)C2)cn2c3c(c(F)c(F)cc13)OCC2]

A mixture of 9,10-difluoro-7-oxo-2,3-dihydro-7H-[1,4]oxazino[2,3,4-ij]quinoline-6-carbaldehyde (300 mg, 1.20 mmol) and NaOAc (98 mg, 1.20 mmol) in DCM/MeOH (3 mL/3 mL) was added (S)-1-(6-nitropyridin-3-yl)piperidin-3-amine (265 mg, 1.20 mmol). The reaction mixture was stirred at 25° C. for 1 hour. NaBH₃CN (300 mg, 4.78 mmol) was added to the reaction mixture in portions at 0° C. The reaction mixture was stirred at 25° C. for 2 hours. LC-MS showed that 9,10-difluoro-7-oxo-2,3-dihydro-7H-[1,4]oxazino[2,3,4-ij]quinoline-6-carbaldehyde was consumed. The reaction mixture was poured into ice-water. Then, the mixture was extracted with DCM/MeOH. The combined organic layer was washed with brine, dried over Na₂SO₄, filtered and concentrated. The crude was purified by column chromatography (SiO₂, DCM:MeOH=20:1) to give (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one (546 mg, 99.6%) as a yellow solid. MS (ESI): mass calcd. For C₂₂H₂₁F₂N₅O₄ 457.16 m/z found 458.1 [M+H]⁺.

### Compound 65

### Preparation of (S)-9,10-difluoro-6-((((2-methoxypyridin-4-yl)methyl)(1-(6-nitropyridin-3-yl)piperidin-3-yl)amino)methyl)-2,3-dihydro-7H-[1,4]oxazino[2,3,4-ij]quinolin-7-one (Step 10 in Scheme 11)

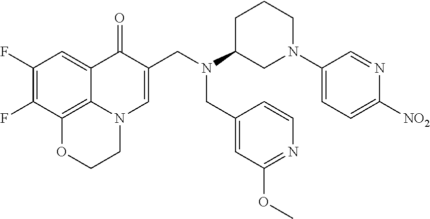 [SMILES: COc1cc(CN(Cc2cn3c4c(c(F)c(F)cc4c2=O)OCC3)[C@H]2CCCN(c3ccc([N+](=O)[O-])nc3)C2)ccn1]

To a solution of (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one (379 mg, 0.83 mmol) in DCE (4 mL) was added 2-methoxyisonicotinaldehyde (341 mg, 2.49 mmol). The reaction mixture was stirred at 25° C. for 1 hour. NaBH(AcO)₃ (527 mg, 2.49 mmol) was added to the mixture in portions at 0° C. The reaction mixture was stirred at 25° C. for 2 hours. LC-MS showed that (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one was consumed. The reaction mixture was poured into ice-water and extracted with EA. The combined organic layer was washed with brine, dried over Na₂SO₄, filtered and concentrated. The crude was purified by column chromatography (SiO₂, DCM:MeOH=15:1) to give (S)-9,10-difluoro-6-((((2-methoxypyridin-4-yl)methyl)(1-(6-nitropyridin-3-yl)piperidin-3-yl)amino)methyl)-2,3-dihydro-7H-[1,4]oxazino[2,3,4-ij]quinolin-7-one (365 mg, 76.2%) as a yellow solid. MS (ESI): mass calcd. For C₂₉H₂₈F₂N₆O₅ 578.21 m/z found 579.3 [M+H]⁺.

### Compound 66

### Preparation of (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (Step 11 in Scheme 11)

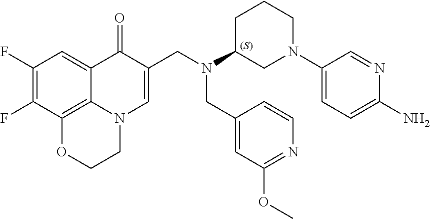

A mixture of(S)-9,10-difluoro-6-((((2-methoxypyridin-4-yl)methyl)(1-(6-nitropyridin-3-yl)piperidin-3-yl)amino)methyl)-2,3-dihydro-7H-[1,4]oxazino[2,3,4-ij]quinolin-7-one (100 mg, 0.17 mmol), C (20 mg) and FeCl₃ (2.8 mg, 0.017 mmol) in EtOH (2 mL) was added NH₂·NH₂·H₂O (0.02 mL, 0.34 mmol). The reaction mixture was stirred at 80° C. for 1.5 hours. LC-MS showed that (S)-9,10-difluoro-6-((((2-methoxypyridin-4-yl)methyl)(1-(6-nitropyridin-3-yl)piperidin-3-yl)amino)methyl)-2,3-dihydro-7H-[1,4]oxazino[2,3,4-ij]quinolin-7-one was consumed. The reaction mixture was diluted with AcOEt and filtered and concentrated. The crude was purified by column chromatography (SiO₂, DCM:MeOH=15:1) to give (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (20 mg, 21.09%) as a yellow solid. MS (ESI): mass calcd. For C₂₉H₃₀F₂N₆O₃ 548.23 m/z found 549.3 [M+H]⁺. ¹H NMR (DMSO-d₆, 400 MHz) δ: 8.00 (d, J=5.3 Hz, 1H), 7.94-7.98 (m, 1H), 7.61 (d, J=2.8 Hz, 1H), 7.47-7.58 (m, 1H), 7.15 (dd, J=8.9, 2.9 Hz, 1H), 6.93-7.06 (m, 1H), 6.80 (s, 1H), 6.39 (d, J=8.8 Hz, 1H), 5.38 (br s, 2H), 4.58 (br t, J=4.4 Hz, 2H), 4.35 (br t, J=4.4 Hz, 2H), 3.77 (s, 3H), 3.70-3.74 (m, 1H), 3.46-3.68 (m, 4H), 3.24 (br d, J=11.4 Hz, 1H), 2.70-2.81 (m, 1H), 2.49-2.53 (m, 1H), 2.35-2.47 (m, 1H), 1.90-2.00 (m, 1H), 1.70-1.78 (m, 1H), 1.38-1.55 (m, 2H).

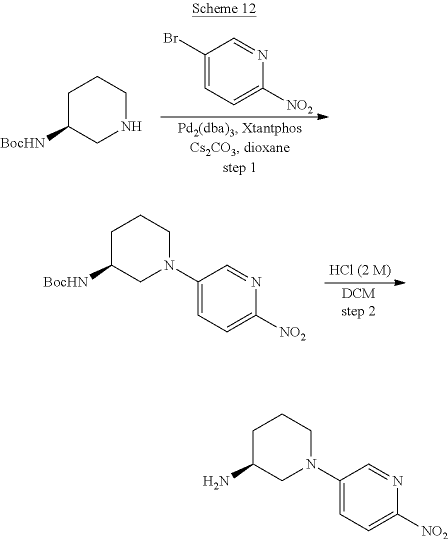 [SMILES: CC(C)(C)OC(=O)N[C@H]1CCCNC1 ; O=[N+]([O-])c1ccc(Br)cn1 ; CC(C)(C)OC(=O)N[C@H]1CCCN(c2ccc([N+](=O)[O-])nc2)C1 ; N[C@H]1CCCN(c2ccc([N+](=O)[O-])nc2)C1]

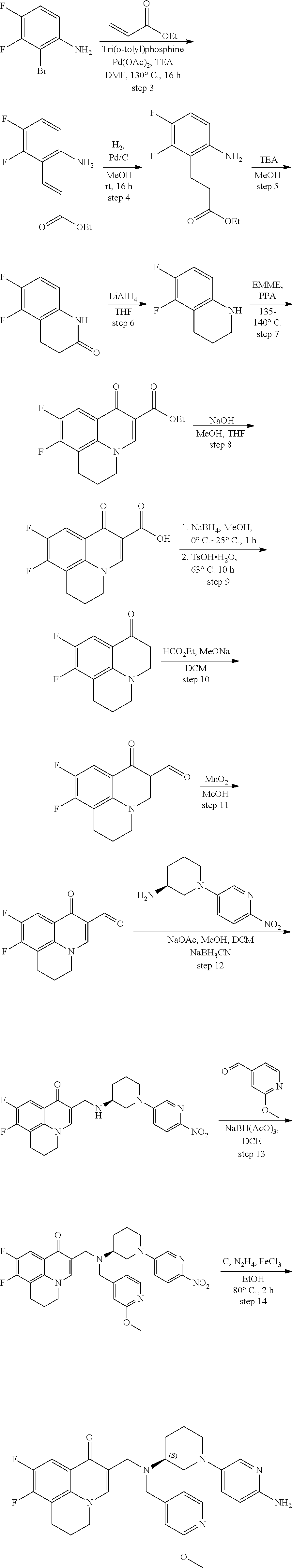

### General Procedures for Preparing Compounds in Scheme 12

### Preparation of 2-methylpropan-2-yl {[(3S)-1-(6-nitropyridin-3-yl) hexahydropyridin-3-yl]amino}methanoate (Step 1 in Scheme 12)

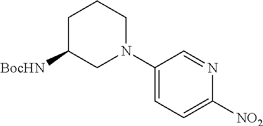 [SMILES: CC(C)(C)OC(=O)N[C@H]1CCCN(c2ccc([N+](=O)[O-])nc2)C1]

A mixture of 5-bromo-2-nitropyridine (4.72 g, 23.4 mmol), 2-methylpropan-2-yl {[(3S)-hexahydropyridin-3-yl]amino}methanoate (3.6 g, 17.97 mmol), Pd₂(dba)₃ (518 mg, 0.9 mmol), Cs₂CO₃ (7.92 g, 24.3 mmol) and [5-(diphenylphosphanyl)-9,9-dimethyl-9H-xanthen-4-yl]diphenylphosphane (625 mg, 1.08 mmol) in dioxane (80 mL) was stirred at 115° C. for 16 hours under N₂ atmosphere. The resulting mixture was poured into ice-water, extracted with EA. The combined organic layer washed with brine, dried over by Na₂SO₄ and concentrated under vacuum. The residue was purified by flash column with EA/PE to afford 2-methylpropan-2-yl {[(3S)-1-(6-nitropyridin-3-yl) hexahydropyridin-3-yl]amino}methanoate (2.9 g, 9.00 mmol) as a yellow solid. MS(ESI): mass calcd. For C₁₅H₂₂N₄O₄ 322.2 m/z found 323.2 [M+H]⁺.

### Preparation of (3S)-1-(6-nitropyridin-3-yl) hexahydropyridin-3-amine (Step 2 in Scheme 12)

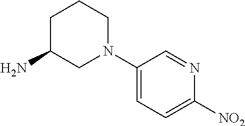 [SMILES: N[C@H]1CCCN(c2ccc([N+](=O)[O-])nc2)C1]

To a solution of 2-methylpropan-2-yl {[(3S)-1-(6-nitropyridin-3-yl) hexahydropyridin-3-yl]amino}methanoate (2.8 g, 8.69 mmol) in DCM (20 mL) was added HCl (26.06 mL, 52.11 mmol) at 0° C. under nitrogen atmosphere. The resulting mixture was stirred at room temperature for 1 hour. The mixture was concentrated under vacuum and diluted with water. The mixture was basified with Na₂CO₃ to pH=8-9. The mixture was extracted with EA. The organic layer was washed with brine, dried over by Na₂SO₄ and concentrated under vacuum. The residue was purified by flash column (eluted with MeOH/DCM) to afford (3S)-1-(6-nitropyridin-3-yl) hexahydropyridin-3-amine (1.6 g, 7.20 mmol, 82.88%) as a yellow solid. MS(ESI): mass calcd. For C₁₀H₁₄N₄O₂ 222.1 m/z found 223.1 [M+H]⁺. ¹H NMR (DMSO-d6) δ: 8.17-8.26 (m, 1H), 8.12 (br d, J=9.3 Hz, 1H), 7.42 (br dd, J=9.3, 2.6 Hz, 1H), 3.86-3.98 (m, 2H), 2.96-3.10 (m, 1H), 2.66-2.83 (m, 2H), 1.69-1.93 (m, 3H), 1.21-1.66 (m, 3H)

### Preparation of ethyl (E)-3-(6-amino-2,3-difluorophenyl) acrylate (Step 3 in Scheme 12)

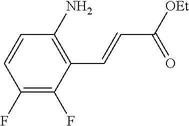 [SMILES: CCOC(=O)/C=C/c1c(N)ccc(F)c1F]

A mixture of ethyl acrylate (14.5 g, 144.9 mmol), 2-bromo-3,4-difluoroaniline (25 g, 120.8 mmol), tris(2-methylphenyl) phosphane (3.66 g, 12.02 mmol), Pd(OAc)₂ (2.7 g, 12.1 mmol) and TEA (49.98 mL, 360.56 mmol) in DMF (300 mL) was stirred at 130° C. for 16 h under nitrogen atmosphere. The resulting mixture was poured into ice-water and extracted with EA. The organic layer was and washed with brine, dried over by Na₂SO₄ and concentrated under vacuum. The residue was purified by flash column (eluted with EA/PE) to afford ethyl (E)-3-(6-amino-2,3-difluorophenyl) acrylate (13 g, 57.22 mmol, 47.61%) as a yellow solid. MS(ESI): mass calcd. For C₁₁H₁₁F₂NO₂ 227.1 m/z found 228.1 [M+H]⁺.

### Preparation of ethyl 3-(6-amino-2,3-difluorophenyl) propanoate (Step 4 in Scheme 12)

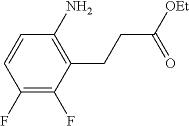 [SMILES: CCOC(=O)CCc1c(N)ccc(F)c1F]

A mixture of ethyl (E)-3-(6-amino-2,3-difluorophenyl) acrylate (13 g, 57.3 mmol) and Pd/C (2.6 g) in MeOH (60 mL) was stirred at room temperature for 16 hours under hydrogen atmosphere. The mixture was filtered and concentrated. The residue was purified by flash column (eluted with EA/PE) to afford ethyl 3-(6-amino-2,3-difluorophenyl) propanoate (11.5 g, 50.22 mmol) as a white solid. MS(ESI): mass calcd. For C₁₁H₁₃F₂NO₂ 229.1 m/z found 230.1 [M+H]⁺.

### Preparation of 5,6-difluoro-3,4-dihydroquinolin-2(1H)-one (Step 5 in Scheme 12)

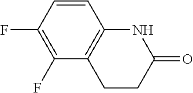 [SMILES: O=C1CCc2c(ccc(F)c2F)N1]

A mixture of ethyl 3-(6-amino-2,3-difluorophenyl) propanoate (7 g, 30.54 mmol) and TEA (6.17 g, 61.07 mmol) in MeOH (50 mL) was stirred at 60° C. for 16 h under nitrogen atmosphere. The resulting mixture was concentrated under vacuum to afford 5,6-difluoro-3,4-dihydroquinolin-2(1H)-one (5 g, 27.30 mmol, 89.39%) as a white solid. MS(ESI): mass calcd. For C₉H₇F₂NO 183.0 m/z found 184.0 [M+H]⁺.

### Preparation of ethyl 8,9-difluoro-1-oxo-6,7-dihydro-1H,5H-pyrido[3,2,1-ij]quinoline-2-carboxylate (Step 6 in Scheme 12)

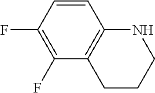 [SMILES: Fc1ccc2c(c1F)CCCN2]

The solution of 5,6-difluoro-3,4-dihydroquinolin-2(1H)-one (9.6 g, 52.41 mmol) and LiAlH₄ (4.97 g, 131.03 mmol) in THF (3 mL) was stirred at room temperature for 2 hours under nitrogen atmosphere. The resulting mixture was poured into ice-water and extracted with EA. The organic layer was washed with brine, dried over by Na₂SO₄ and concentrated under vacuum. The residue was purified by FC with EA/PE to afford 5,6-difluoro-1,2,3,4-tetrahydroquinoline (8 g, 47.29 mmol, 90.22%) as a yellow solid. MS(ESI): mass calcd. For C₉H₉F₂N 169.1 m/z found 170.1 [M+H]⁺.

### Preparation of 5,6-difluoro-1,2,3,4-tetrahydroquinoline (Step 7 in Scheme 12)

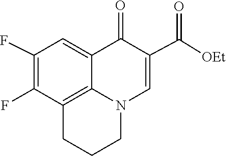 [SMILES: CCOC(=O)c1cn2c3c(c(F)c(F)cc3c1=O)CCC2]

A mixture of 5,6-difluoro-1,2,3,4-tetrahydroquinoline (8 g, 47.29 mmol) and ethyl 3-ethoxy-2-(ethoxycarbonyl) prop-2-enoate (11.36 mL, 56.75 mmol) was stirred at 130° C. for 1 h under nitrogen atmosphere. Then, oxo-k5-phosphanediol (38.77 g, 472.90 mmol) was added. The reaction mixture was stirred at 130° C. for 2 h under nitrogen atmosphere. The resulting mixture was poured into ice-water. The mixture was filtered. The resulting filter cake was collected to afford ethyl 8,9-difluoro-1-oxo-6,7-dihydro-1H,5H-pyrido[3,2,1-ij]quinoline-2-carboxylate (11 g, 37.51 mmol, 79.32%) as a gray solid. MS(ESI): mass calcd. For C₁₅H₁₃F₂NO₃ 293.1 m/z found 294.1 [M+H]⁺.

### Preparation of 8,9-difluoro-1-oxo-6,7-dihydro-1H,5H-pyrido[3,2,1-ij]quinoline-2-carboxylic Acid (Step 8 in Scheme 12)

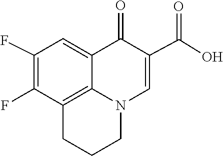 [SMILES: O=C(O)c1cn2c3c(c(F)c(F)cc3c1=O)CCC2]

A mixture of ethyl 8,9-difluoro-1-oxo-6,7-dihydro-1H,5H-pyrido[3,2,1-ij]quinoline-2-carboxylate (11 g, 37.51 mmol) and sodium hydroxide (4.50 g, 112.52 mmol) in MeOH (30 mL) and THF (30 mL) was stirred at 50° C. for 1 h under nitrogen atmosphere. The resulting mixture was poured into ice-water. The mixture was acidified to pH=2 with HCl. The resulting mixture was extracted with EA. The organic layer was washed with brine, dried over by Na₂SO₄ and concentrated under vacuum to afford 8,9-difluoro-1-oxo-6,7-dihydro-1H,5H-pyrido[3,2,1-ij]quinoline-2-carboxylic acid (9.5 g, 35.82 mmol, 95.50%) as a gray solid. MS(ESI): mass calcd. For C₁₃H₉F₂NO₃ 265.1 m/z found 266.1 [M+H]⁺. ¹H NMR (DMSO-d6) δ: 14.99 (br s, 1H), 8.97 (s, 1H), 8.10 (br t, J=9.3 Hz, 1H), 4.44 (br s, 2H), 3.04 (br s, 2H), 2.17 (br s, 2H)

### Preparation of 8,9-difluoro-2,3,6,7-tetrahydro-1H,5H-pyrido[3,2,1-ij]quinolin-1-one (Step 9 in Scheme 12)

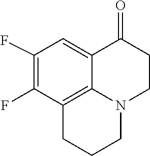 [SMILES: O=C1CCN2CCCc3c(F)c(F)cc1c32]

To a solution of 8,9-difluoro-1-oxo-6,7-dihydro-1H,5H-pyrido[3,2,1-ij]quinoline-2-carboxylic acid (4 g, 15.08 mmol) in MeOH (20 mL) was added NaBH₄ (1.66 mL, 45.25 mmol) at 0° C. under nitrogen atmosphere. The resulting solution was stirred at room temperature for 1 hour. 4-methylbenzenesulfonic acid (260 mg, 1.51 mmol) was added to the mixture. The reaction mixture was stirred at 63° C. for 16 h. The mixture was poured into ice-water and extracted with EA. The organic layer was washed with brine, dried over Na₂SO₄ and concentrated under vacuum. The residue was purified by flash column (eluted with EA/PE) to afford 8,9-difluoro-2,3,6,7-tetrahydro-1H,5H-pyrido[3,2,1-ij]quinolin-1-one (2.7 g, 12.10 mmol, 80.20%) as a yellow oil. MS(ESI): mass calcd. For C₁₂H₁₁F₂NO 223.1 m/z found 224.1 [M+H]⁺.

### Preparation of 8,9-difluoro-1-oxo-2,3,6,7-tetrahydro-1H,5H-pyrido[3,2,1-ij]quinoline-2-carbaldehyde (Step 10 in Scheme 12)

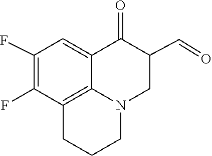 [SMILES: O=CC1CN2CCCc3c(F)c(F)cc(c32)C1=O]

To a solution of 8,9-difluoro-2,3,6,7-tetrahydro-1H,5H-pyrido[3,2,1-ij]quinolin-1-one (2.7 g, 12.10 mmol) in DCM (3 mL) was added sodium methanolate (2.61 g, 48.38 mmol) and ethyl methanoate (3.90 mL, 48.38 mmol) at room temperature under N₂ atmosphere. The resulting mixture was stirred at room temperature for 10 minutes. The mixture was filtered. The filter cake was collected to afford 8,9-difluoro-1-oxo-2,3,6,7-tetrahydro-1H,5H-pyrido[3,2,1-ij]quinoline-2-carbaldehyde (2.5 g, 9.95 mmol, 82.27%) as a gray solid. MS(ESI): mass calcd. For C₁₃H₁₁F₂NO₂ 251.1 m/z found 252.1 [M+H]⁺.

### Preparation of 8,9-difluoro-1-oxo-6,7-dihydro-1H,5H-pyrido[3,2,1-ij]quinoline-2-carbaldehyde (Step 11 in Scheme 12)

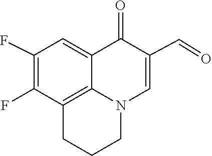 [SMILES: O=Cc1cn2c3c(c(F)c(F)cc3c1=O)CCC2]

A mixture of 8,9-difluoro-1-oxo-2,3,6,7-tetrahydro-1H,5H-pyrido[3,2,1-ij]quinoline-2-carbaldehyde (2.5 g, 9.95 mmol) and manganese dioxide (953 mg, 10.95 mmol) in MeOH (30 mL) was stirred at room temperature for 16 hours under N₂ atmosphere. The mixture was filtered. The filtrate was collected and concentrated. The residue was washed with EA and filtered. The filter cake was collected to afford 8,9-difluoro-1-oxo-6,7-dihydro-1H,5H-pyrido[3,2,1-ij]quinoline-2-carbaldehyde (1.2 g, 4.82 mmol, 48.39%) as a brown solid. MS(ESI): mass calcd. For C₁₃H₉F₂NO₂ 249.1 m/z found 250.1 [M+H]⁺.

### Preparation of (S)-8,9-difluoro-2-(((1-(6-nitropyridin-3-yl)piperidin-3-yl) amino)methyl)-6,7-dihydro-1H,5H-pyrido[3,2,1-ij]quinolin-1-one (Step 12 in Scheme 12)

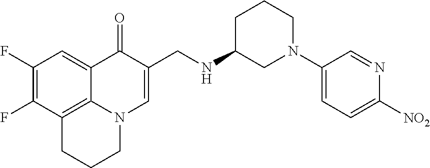 [SMILES: O=c1c(CN[C@H]2CCCN(c3ccc([N+](=O)[O-])nc3)C2)cn2c3c(c(F)c(F)cc13)CCC2]

A mixture of 8,9-difluoro-1-oxo-6,7-dihydro-1H,5H-pyrido[3,2,1-ij]quinoline-2-carbaldehyde (450 mg, 1.81 mmol), NaOAc (148.12 mg, 1.81 mmol) and (3S)-1-(6-nitropyridin-3-yl)hexahydropyridin-3-amine (401.30 mg, 1.81 mmol) in DCM (10 mL) and MeOH (1 mL) was stirred at room temperature for 2 hours under N₂ atmosphere. Sodium cyanoboranuide (340.40 mg, 5.42 mmol) was then added to the mixture, the resulting mixture was stirred at room temperature for 4 hours. The mixture was poured into ice-water and extracted with EA. The organic layer was washed with brine, dried over Na₂SO₄ and concentrated under vacuum. The residue was purified by flash column (eluted with MeOH/DCM) to afford (S)-8,9-difluoro-2-(((1-(6-nitropyridin-3-yl)piperidin-3-yl) amino)methyl)-6,7-dihydro-1H,5H-pyrido[3,2,1-ij]quinolin-1-one (600 mg, 1.32 mmol, 72.96%) as a yellow solid. MS(ESI): mass calcd. For C₂₃H₂₃F₂N₅O₃ 455.2 m/z found 456.2 [M+H]⁺.

### Preparation of (S)-8,9-difluoro-2-((((2-methoxypyridin-4-yl)methyl)(1-(6-nitropyridin-3-yl)piperidin-3-yl)amino)methyl)-6,7-dihydro-1H,5H-pyrido[3,2,1-ij]quinolin-1-one (Step 13 in Scheme 12)

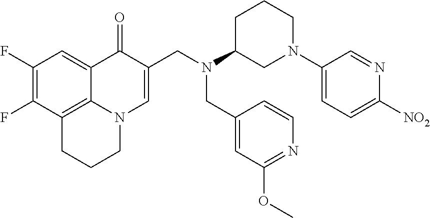 [SMILES: COc1cc(CN(Cc2cn3c4c(c(F)c(F)cc4c2=O)CCC3)[C@H]2CCCN(c3ccc([N+](=O)[O-])nc3)C2)ccn1]

A mixture of (S)-8,9-difluoro-2-(((1-(6-nitropyridin-3-yl)piperidin-3-yl)amino) methyl)-6,7-dihydro-1H,5H-pyrido[3,2,1-ij]quinolin-1-one (300 mg, 0.66 mmol) and 2-methoxypyridine-4-carbaldehyde (270.99 mg, 1.98 mmol) in DCE (10 mL) was stirred at room temperature for 2 hours under N₂ atmosphere. NaBH(OAc)₃ (418.91 mg, 1.98 mmol) was then added to the mixture, the resulting mixture was stirred at room temperature for 4 hours. The mixture was poured into ice-water and extracted with EA. The organic layer was washed with brine, dried over Na₂SO₄ and concentrated under vacuum. The residue was purified by flash column (eluted with MeOH/DCM) to afford (S)-8,9-difluoro-2-((((2-methoxypyridin-4-yl)methyl)(1-(6-nitropyridin-3-yl)piperidin-3-yl)amino)methyl)-6,7-dihydro-1H,5H-pyrido[3,2,1-ij]quinolin-1-one (115 mg, 0.20 mmol, 30.28%) as a yellow solid. MS(ESI): mass calcd. For C₃₀H₃₀F₂N₆O₄ 576.2 m/z found 577.2 [M+H]⁺.

### Compound 67

### Preparation of (S)-2-(((1-(6-aminopyridin-3-yl)piperidin-3-yl)((2-methoxypyridin-4-yl)methyl)amino)methyl)-8,9-difluoro-6,7-dihydro-1H,5H-pyrido[3,2,1-ij]quinolin-1-one (Step 14 in Scheme 12)

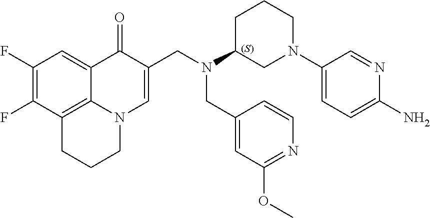

A mixture of (S)-8,9-difluoro-2-((((2-methoxypyridin-4-yl)methyl)(1-(6-nitropyridin-3-yl)piperidin-3-yl)amino)methyl)-6,7-dihydro-1H,5H-pyrido[3,2,1-ij]quinolin-1-one (100 mg, 0.17 mmol), C (20 mg, 1.67 mmol), FeCl₃ (2.81 mg, 0.02 mmol) and N₂H₄(27.75 mg, 0.87 mmol) in EtOH (10 mL) was stirred at 80° C. for 2 hours under N₂ atmosphere. The mixture was filtered. The filtrate was collected and concentrated. The residue was purified by flash column (eluted with MeOH/DCM) to afford (S)-2-(((1-(6-aminopyridin-3-yl)piperidin-3-yl)((2-methoxypyridin-4-yl)methyl)amino)methyl)-8,9-difluoro-6,7-dihydro-1H,5H-pyrido[3,2,1-ij]quinolin-1-one (12 mg, 0.02 mmol, 12.66%) as a white solid. MS(ESI): mass calcd. For C₃₀H₃₂F₂N₆O₂ 546.3 m/z found 547.3 [M+H]⁺. ¹H NMR (DMSO-d6) δ: 7.99 (d, J=5.3 Hz, 1H), 7.94 (s, 1H), 7.83 (dd, J=10.7, 9.1 Hz, 1H), 7.60 (d, J=2.9 Hz, 1H), 7.14 (dd, J=8.8, 2.9 Hz, 1H), 6.98 (dd, J=5.3, 1.0 Hz, 1H), 6.78 (s, 1H), 6.37 (d, J=8.9 Hz, 1H), 5.37 (s, 2H), 4.16 (br s, 2H), 3.76 (s, 3H), 3.71 (d, J=3.8 Hz, 1H), 3.68-3.75 (m, 1H), 3.55-3.65 (m, 2H), 3.44-3.53 (m, 1H), 3.23 (br d, J=11.5 Hz, 1H), 2.95 (br t, J=5.9 Hz, 2H), 2.75 (br s, 1H), 2.55-2.62 (m, 1H), 2.41 (br s, 1H), 2.04-2.14 (m, 2H), 1.96 (br d, J=10.4 Hz, 1H), 1.71-1.79 (m, 1H), 1.37-1.56 (m, 2H).

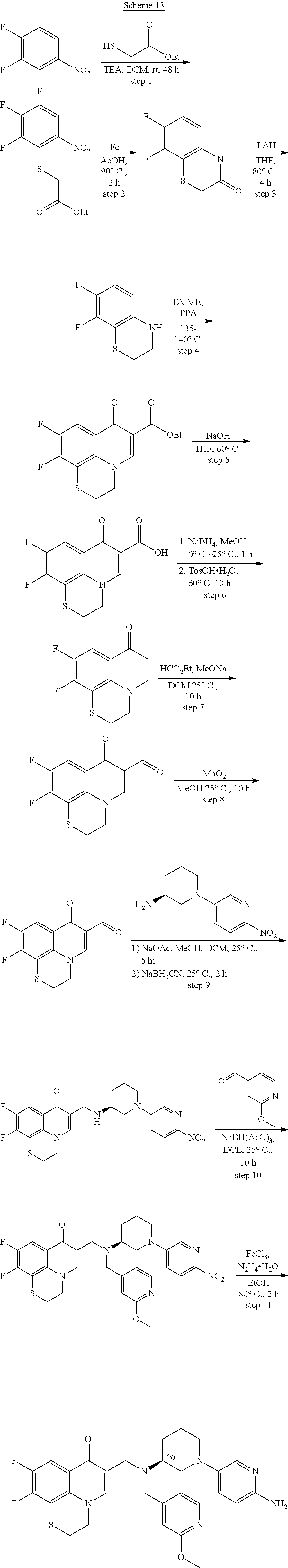

### General Procedures for Preparing Compounds in Scheme 13

### Preparation of ethyl 2-((2,3-difluoro-6-nitrophenyl)thio)acetate (Step 1 in Scheme 13)

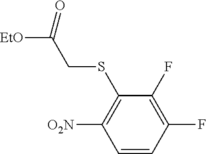 [SMILES: CCOC(=O)CSc1c([N+](=O)[O-])ccc(F)c1F]

A mixture of dioxo(2,3,4-trifluorophenyl)-λ⁵-azane (13.07 mL, 112.94 mmol) and TEA (31.31 mL, 225.89 mmol) in DCM (100 mL) were added ethyl sulfanyl acetate (12.45 mL, 112.94 mmol) under nitrogen atmosphere. The reaction mixture was stirred at room temperature for 2 days. TLC (PE:EA=10:1) showed the reaction was completed. The mixture was poured into H₂O (300 mL) and extracted with DCM (100 mL×3). The combined organic layer was washed with brine (100 mL×1), dried over Na₂SO₄ and filtrated. The filtrate was concentrated. The residue was purified by column chromatography (SiO₂, PE:EA=10:1-5:1) to give ethyl 2-((2,3-difluoro-6-nitrophenyl)thio)acetate (16 g, 51% yield) as a yellow solid.

### Preparation of 7,8-difluoro-2H-benzo[b][1,4]thiazin-3(4H)-one (Step 2 in Scheme 13)

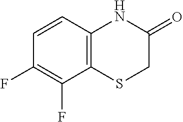 [SMILES: O=C1CSc2c(ccc(F)c2F)N1]

To a solution of ethyl 2-((2,3-difluoro-6-nitrophenyl)thio)acetate (13 g, 46.89 mmol) in AcOH (50 mL) was added Fe (7.86 g, 140.67 mmol) at room temperature under N₂ atmosphere. The reaction mixture was stirred at 90° C. for 2 hours. TLC (PE:EA=2:1) showed the reaction was completed. The reaction mixture was cooled to room temperature and diluted with EA (150 mL). the mixture was filtrated. The filter cake was washed with EA (100 mL×3). The mixture was poured into H₂O (300 mL). The mixture was basified to pH=8-9 with NaHCO₃ and extracted with EA (150 mL×2). The combined organic layer was washed with brine (150 mL), dried over Na₂SO₄, filtrated and concentrated. The residue was purified by column chromatography (SiO₂, PE:EA=2:1) to give 7,8-difluoro-2H-benzo[b][1,4]thiazin-3(4H)-one (7.5 g, 80% yield) as a yellow solid.

### Preparation of 7,8-difluoro-3,4-dihydro-2H-benzo[b][1,4]thiazine (Step 3 in Scheme 13)

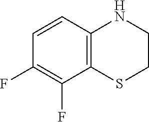 [SMILES: Fc1ccc2c(c1F)SCCN2]

To a solution of 7,8-difluoro-2H-benzo[b][1,4]thiazin-3(4H)-one (9.0 g, 44.73 mmol) in THF (120 mL) was added LiAlH₄ (5.09 g, 134.20 mmol) at 0° C. under N₂ atmosphere. The mixture was stirred at 75° C. for 2 hours. The reaction mixture was cooled to 0° C. and quenched with H₂O 5 mL and aq.NaOH (5 mL, 1M). The mixture was diluted with THF (100 mL) and filtrated. The filter cake washed with EA (100 mL×6). The filtrate was diluted with H₂O (200 mL) and extracted with EA (100 mL×3). The combined organic layer was washed with brine (100 mL×1), dried over Na₂SO₄, filtered and concentrated. The crude was purified by column chromatography (SiO₂, PE:EA=10:1-2:1) to give 7,8-difluoro-3,4-dihydro-2H-benzo[b][1,4]thiazine (7.1 g, 84% yield) was as a yellow solid. ¹H NMR (DMSO-d6) δ: 6.87 (dt, J=10.4, 9.1 Hz, 1H), 6.21-6.35 (m, 2H), 3.39-3.50 (m, 2H), 2.94-3.05 (m, 2H)

### Preparation of ethyl 9,10-difluoro-7-oxo-2,3-dihydro-7H-[1,4]thiazino[2,3,4-ij]quinoline-6-carboxylate (Step 4 in Scheme 13)

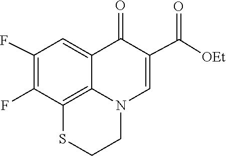 [SMILES: CCOC(=O)c1cn2c3c(c(F)c(F)cc3c1=O)SCC2]

A mixture of 7,8-difluoro-3,4-dihydro-2H-benzo[b][1,4]thiazine (6.0 g, 32.05 mmol) and EMME (7.62 g, 35.25 mmol) was heated at 120° C. for 2 hours. Polyphosphoric acid (30 g) was added. The mixture was then gradually heated to 160° C. for 1 hour. TLC (DCM:MeOH=20:1) showed the reaction was completed. The reaction mixture was poured into H₂O (100 mL) and stirred at room temperature for 30 min. The mixture was filtrated. The filter cake was washed and dried to give ethyl 9,10-difluoro-7-oxo-2,3-dihydro-7H-[1,4]thiazino[2,3,4-ij]quinoline-6-carboxylate (9.8 g, 98.2 yield) as a yellow solid.

### Preparation of 9,10-difluoro-7-oxo-2,3-dihydro-7H-[1,4]thiazino[2,3,4-ij]quinoline-6-carboxylic acid (Step 5 in Scheme 13)

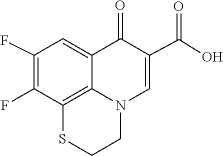 [SMILES: O=C(O)c1cn2c3c(c(F)c(F)cc3c1=O)SCC2]

A mixture of ethyl 9,10-difluoro-7-oxo-2,3-dihydro-7H-[1,4]thiazino[2,3,4-ij]quinoline-6-carboxylate (9.8 g, 31.48 mmol) in THF (150 mL) and MeOH (30 mL) was added aq. LiOH (2.52 g, 62.96 mmol, 4M). The mixture was heated to 50° C. for 1 hour. The mixture was cooled to room temperature and concentrated. The residue was diluted with H₂O (100 mL). The mixture was acidified to PH=4-5 and filtrated. The filter cake was washed with H₂O (100 mL) and dried to give 9,10-difluoro-7-oxo-2,3-dihydro-7H-[1,4]thiazino[2,3,4-ij]quinoline-6-carboxylic acid (7.5 g, 84% yield) as a grey solid.

### Preparation of 9,10-difluoro-2,3,5,6-tetrahydro-7H-[1,4]thiazino[2,3,4-ij]quinolin-7-one (Step 6 in Scheme 13)

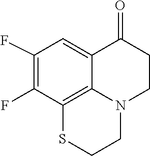 [SMILES: O=C1CCN2CCSc3c(F)c(F)cc1c32]

A mixture of 9,10-difluoro-7-oxo-2,3-dihydro-7H-[1,4]thiazino[2,3,4-ij]quinoline-6-carboxylic acid (1.8 g, 6.35 mmol) in MeOH (80 mL) was added NaBH₄ (1.06 g, 27.96 mmol) at 0° C. The reaction mixture was stirred at room temperature for 1 hour. TsOH·H₂O (0.15 g, 0.88 mmol) was added to the mixture. The mixture was stirred at 60° C. overnight. The mixture was concentrated. The residue was diluted with DCM (50 mL) and H₂O (20 mL) and extracted with DCM (20 mL×3). The combined organic layer was washed with brine (25 mL), dried over Na₂SO₄, filtered and concentrated. The residue was purity by column chromatography (SiO₂, DCM:EA=5:1) to give 9,10-difluoro-2,3,5,6-tetrahydro-7H-[1,4]thiazino[2,3,4-ij]quinolin-7-one (1.18 g, 4.89 mmol, 77.12% yield) as a yellow solid.

### Preparation of 9,10-difluoro-7-oxo-2,3,6,7-tetrahydro-5H-[1,4]thiazino[2,3,4-ij]quinoline-6-carbaldehyde (Step 7 in Scheme 13)

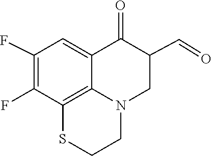 [SMILES: O=CC1CN2CCSc3c(F)c(F)cc(c32)C1=O]

A mixture of 9,10-difluoro-2,3,5,6-tetrahydro-7H-[1,4]thiazino[2,3,4-ij]quinolin-7-one (1.18 g, 4.89 mmol) and MeONa (1.06 g, 19.56 mmol) in DCM (30 mL) was added ethyl methanoate (1.50 mL, 18.58 mmol) at 0° C. The reaction was stirred at room temperature for 30 minutes. The reaction mixture was diluted with H₂O (30 mL) and extracted with DCM (50 mL×3). The combined organic layer was washed with brine (25 mL), dried over Na₂SO₄, filtered and concentrated. The residue was purity by column chromatography (SiO₂, DCM:EA=10:1) to give 9,10-difluoro-7-oxo-2,3,6,7-tetrahydro-5H-[1,4]thiazino[2,3,4-ij]quinoline-6-carbaldehyde (1.0 g, 3.71 mmol, 75.76% yield) as a red solid.

### Preparation of 9,10-difluoro-7-oxo-2,3-dihydro[1,4]thiazino[2,3,4-ij]quinoline-6-carbaldehyde (Step 8 in Scheme 13)

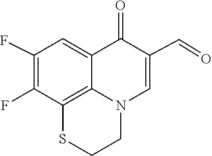 [SMILES: O=Cc1cn2c3c(c(F)c(F)cc3c1=O)SCC2]

To a solution of 9,10-difluoro-7-oxo-2,3,6,7-tetrahydro-5H-[1,4]thiazino[2,3,4-ij]quinoline-6-carbaldehyde (0.8 g, 2.97 mmol) in MeOH (30 mL) was added MnO₂ (0.38 g, 4.46 mmol) at room temperature. The reaction mixture was stirred at room temperature overnight. The mixture was filtrated. The filter cake was washed with DCM. The combined organic layer was concentrated and purified by chromatography on silica gel DCM:MeOH=10:1 to give 9,10-difluoro-7-oxo-2,3-dihydro[1,4]thiazino[2,3,4-ij]quinoline-6-carbaldehyde (0.42 g, 1.57 mmol, 53.16%) as a yellow solid.

### Preparation of (S)-9,10-difluoro-6-(((1-(6-nitropyridin-3-yl)piperidin-3-yl)amino)methyl)-2,3-dihydro-7H-[1,4]thiazino[2,3,4-ij]quinolin-7-one (Step 9 in Scheme 13)

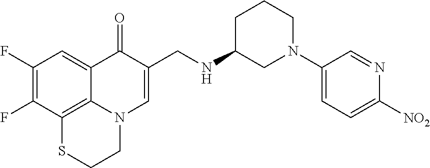 [SMILES: O=c1c(CN[C@H]2CCCN(c3ccc([N+](=O)[O-])nc3)C2)cn2c3c(c(F)c(F)cc13)SCC2]

A mixture of (3S)-1-(6-nitropyridin-3-yl)hexahydropyridin-3-amine (299.38 mg, 1.35 mmol) and 9,10-difluoro-7-oxo-2,3-dihydro[1,4]thiazino[2,3,4-ij]quinoline-6-carbaldehyde (400 mg, 1.50 mmol) in DCM (20 mL) was added AcONa (122.73 mg, 1.50 mmol) at room temperature under N₂ atmosphere. The reaction was stirred at room temperature for 3 hours. NaBH₃CN (103.46 mg, 1.65 mmol) was added to the mixture. The reaction mixture was stirred at room temperature overnight. The reaction mixture was acidified to pH=5-6 with 1N HCl and extracted with DCM (20 mL×3). The combined organic layer was washed with brine (20 mL), dried over Na₂SO₄, filtered and concentrated. The residue was purified by column chromatography (SiO₂, DCM:MeOH=10:1) to give (S)-9,10-difluoro-6-(((1-(6-nitropyridin-3-yl)piperidin-3-yl)amino)methyl)-2,3-dihydro-7H-[1,4]thiazino[2,3,4-ij]quinolin-7-one (378 mg, 0.80 mmol, 53.24%) as a yellow solid.

### Compound 68

### Preparation of 9,10-difluoro-6-({[(2-methoxypyridin-4-yl)methyl][(3S)-1-(6-nitropyridin-3-yl)hexahydropyridin-3-yl]amino}methyl)-3,7-dihydro-2H-[1,4]thiazino[2,3,4-ij]quinolin-7-one (Step 10 in Scheme 13)

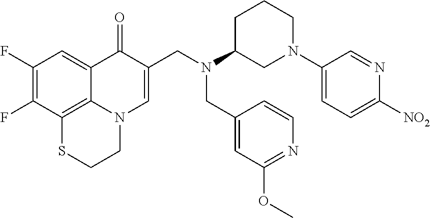 [SMILES: COc1cc(CN(Cc2cn3c4c(c(F)c(F)cc4c2=O)SCC3)[C@H]2CCCN(c3ccc([N+](=O)[O-])nc3)C2)ccn1]

A mixture of (S)-9,10-difluoro-6-(((1-(6-nitropyridin-3-yl)piperidin-3-yl)amino)methyl)-2,3-dihydro-7H-[1,4]thiazino[2,3,4-ij]quinolin-7-one (200 mg, 0.42 mmol) and 2-methoxypyridine-4-carbaldehyde (173 mg, 1.26 mmol) in DCM (20 mL) was added AcOH (2 drops) at room temperature under N₂ atmosphere. The reaction was stirred at room temperature for 3 hours. NaBH(OAc)₃ (211 mg, 1.27 mmol) was added to the reaction mixture. The mixture was stirred at room temperature overnight. The reaction mixture was adjusted to pH=7-8 with NaHCO₃. The mixture was extracted with DCM (20 mL×3). The combined organic layer was washed with brine (20 mL), dried over Na₂SO₄, filtered and concentrated. The yellow residue was purified by column chromatography (SiO₂, DCM:MeOH=10:1) to give 9,10-difluoro-6-({[(2-methoxypyridin-4-yl)methyl][(3S)-1-(6-nitropyridin-3-yl)hexahydropyridin-3-yl]amino}methyl)-3,7-dihydro-2H-[1,4]thiazino[2,3,4-ij]quinolin-7-one (140 mg, 0.24 mmol, 56.00% yield) as a yellow solid.

### Compound 69

### Preparation of 9,10-difluoro-6-({[(3S)-1-(6-aminopyridin-3-yl)hexahydropyridin-3-yl][(2-methoxypyridin-4-yl)methyl]amino}methyl)-3,7-dihydro-2H-[1,4]thiazino[2,3,4-ij]quinolin-7-one (Step 11 in Scheme 13)

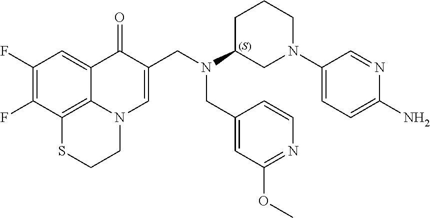

A mixture of 9,10-difluoro-6-({[(2-methoxypyridin-4-yl)methyl][(3S)-1-(6-nitropyridin-3-yl)hexahydropyridin-3-yl]amino}methyl)-3,7-dihydro-2H-[1,4]thiazino[2,3,4-ij]quinolin-7-one (140 mg, 0.24 mmol), FeCl₃ (3.82 mg, 0.02 mmol) and C (0.02 mL, 2.35 mmol) in EtOH (10 mL) was added NH₂·NH₂·H₂O (131.84 mg, 2.35 mmol) at room temperature. The reaction was stirred at 80° C. for 2 hours. TLC (DCM:MeOH=10:1) showed the reaction was completed. The mixture was filtered. The filter cake was washed with DCM. The combined organic layer was concentrated and purified by Pre-HPLC to give 9,10-difluoro-6-({[(3S)-1-(6-aminopyridin-3-yl)hexahydropyridin-3-yl][(2-methoxypyridin-4-yl)methyl]amino}methyl)-3,7-dihydro-2H-[1,4]thiazino[2,3,4-ij]quinolin-7-one (38 mg, 0.07 mmol, 29.23% yield) as a white solid which was confirmed by LCMS/H NMR. ¹H NMR (DMSO-d⁶) δ: 7.98 (d, J=5.3 Hz, 1H), 7.91 (s, 1H), 7.72-7.80 (m, 1H), 7.60 (d, J=2.8 Hz, 1H), 7.14 (dd, J=8.8, 2.9 Hz, 1H), 6.97 (d, J=4.8 Hz, 1H), 6.76 (s, 1H), 6.38 (d, J=8.8 Hz, 1H), 5.37 (s, 2H), 4.46 (br d, J=4.3 Hz, 2H), 3.75 (s, 3H), 3.71 (br d, J=3.4 Hz, 2H), 3.59 (br d, J=5.9 Hz, 2H), 3.48 (br d, J=9.6 Hz, 1H), 3.37-3.43 (m, 2H), 3.23 (br d, J=11.4 Hz, 1H), 2.70-2.81 (m, 1H), 1.96 (br d, J=11.0 Hz, 1H), 1.75 (br d, J=12.4 Hz, 1H), 1.41 (m, 2H). MS (ESI): mass calcd. For C₂₉H₃₀F₂N₆O₂S 564.21 m/z found 565.3[M+H]⁺.

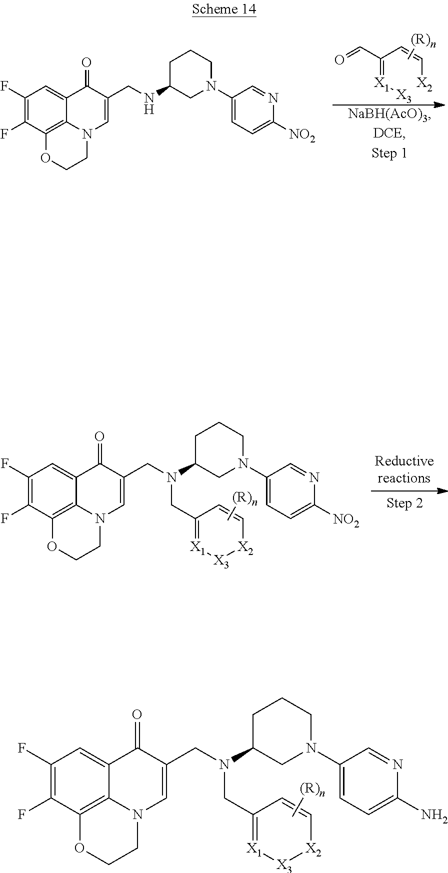

### General Procedures for Preparing Compounds in Scheme 14

### Preparation of Compounds in Scheme 14 (Step 1 in Scheme 14)

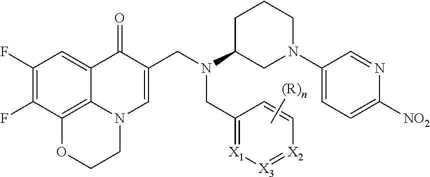

To a solution of (S)-9,10-difluoro-6-(((1-(6-nitropyridin-3-yl)piperidin-3-yl) amino)methyl)-2,3-dihydro-7H-[1,4]oxazino [2,3,4-ij]quinolin-7-one (1 eq) in DCE (3 mL) was added aldehyde derivatives (3 eq). The reaction mixture was stirred at 25° C. for 1 hour. NaBH(AcO)₃ (278 mg, 1.32 mmol) was added to the mixture in portion at 0° C. The reaction mixture was stirred at 25° C. for 2 hours. LCMS showed that (S)-9,10-difluoro-6-(((1-(6-nitropyridin-3-yl)piperidin-3-yl)amino) methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude was purified to give desired product.

### Preparation of (S)-9,10-difluoro-6-(((1-(6-nitropyridin-3-yl)piperidin-3-yl)(pyridin-4-ylmethyl)amino)methyl)-2,3-dihydro-7H-[1,4]oxazino[2,3,4-ij]quinolin-7-one (Step 1 in Scheme 14)

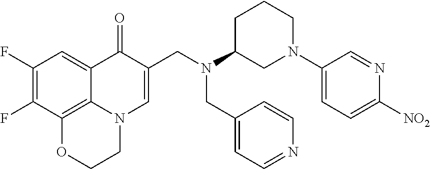 [SMILES: O=c1c(CN(Cc2ccncc2)[C@H]2CCCN(c3ccc([N+](=O)[O-])nc3)C2)cn2c3c(c(F)c(F)cc13)OCC2]

To a solution of (S)-9,10-difluoro-6-(((1-(6-nitropyridin-3-yl)piperidin-3-yl) amino)methyl)-2,3-dihydro-7H-[1,4]oxazino [2,3,4-ij]quinolin-7-one (200 mg, 0.44 mmol) in DCE (3 mL) was added isonicotinaldehyde (141 mg, 1.32 mmol). The reaction mixture was stirred at 25° C. for 1 hour. NaBH(AcO)₃ (278 mg, 1.32 mmol) was added to the mixture in portion at 0° C. The reaction mixture was stirred at 25° C. for 2 hours. LCMS showed that (S)-9,10-difluoro-6-(((1-(6-nitropyridin-3-yl)piperidin-3-yl)amino) methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=20:1) to give (S)-9,10-difluoro-6-(((1-(6-nitropyridin-3-yl)piperidin-3-yl)(pyridin-4-yl-methyl)amino) methyl)-2,3-dihydro-7H-[1,4]oxazino[2,3,4-ij]quinolin-7-one (208 mg, 86.18% yield) as a yellow solid. MS (ESI): mass calcd. For C₂₈H₂₆F₂N₆O₄ 548.2 m/z found 549.3 [M+H]⁺.

### Preparation of (S)-9,10-difluoro-6-((((2-methylpyridin-4-yl)methyl)(1-(6-nitropyridin-3-yl)piperidin-3-yl)amino)methyl)-2,3-dihydro-7H-[1,4]oxazino[2,3,4-ij]quinolin-7-one (Step 1 in Scheme 14)

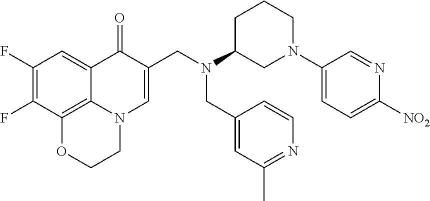 [SMILES: Cc1cc(CN(Cc2cn3c4c(c(F)c(F)cc4c2=O)OCC3)[C@H]2CCCN(c3ccc([N+](=O)[O-])nc3)C2)ccn1]

To a solution of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) in DCE (1 mL) was added 2-methylisonicotinaldehyde (80 mg, 0.66 mmol). The reaction mixture was stirred at 25° C. for 1 hour. NaBH(AcO)₃ (140 mg, 0.66 mmol) was added to the mixture in portion at 0° C. The reaction mixture was stirred at 25° C. for 2 hours. LCMS showed that (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=15:1) to give (S)-9,10-difluoro-6-((((2-methylpyridin-4-yl)methyl) (1-(6-nitropyridin-3-yl)piperidin-3-yl)amino)methyl)-2,3-dihydro-7H-[1,4]oxazino[2,3,4-ij]quinolin-7-one (110 mg, 88.88% yield) as a yellow solid. MS (ESI): mass calcd. For C₂₉H₂₈F₂N₆O₄ 562.21 m/z found 563.3 [M+H]⁺.

### Preparation of (S)-6-((benzyl(1-(6-nitropyridin-3-yl)piperidin-3-yl)amino)methyl)-9,10-difluoro-2,3-dihydro-7H-[1,4]oxazino[2,3,4-ij]quinolin-7-one (Step 1 in Scheme 14)

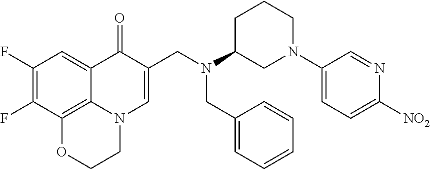 [SMILES: O=c1c(CN(Cc2ccccc2)[C@H]2CCCN(c3ccc([N+](=O)[O-])nc3)C2)cn2c3c(c(F)c(F)cc13)OCC2]

To a solution of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) in DCE (1 mL) was added benzaldehyde (0.07 mL, 0.66 mmol). The reaction mixture was stirred at 25° C. for 1 hour. NaBH(AcO)₃ (140 mg, 0.66 mmol) was added to the mixture in portion at 0° C. The reaction mixture was stirred at 25° C. for 2 hours. LCMS showed that (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one was consumed. The reaction mixture was poured into ice-water and extracted with AcOEt. The combined organic layer was washed with brine, dried over Na₂SO₄, filtered and concentrated. The crude product was purified by column chromatography (SiO₂, DCM:MeOH=20:1) to give (S)-6-((benzyl(1-(6-nitropyridin-3-yl)piperidin-3-yl)amino)methyl)-9,10-difluoro-2,3-dihydro-7H-[1,4]oxazino[2,3,4-ij]quinolin-7-one (88 mg, 73.52%) as a yellow solid. MS (ESI): mass calcd. For C₂₉H₂₇F₂N₅O₄ 547.20 m/z found 548.3 [M+H]⁺.

### Preparation of (S)-6-(((2,4-dimethoxybenzyl)(1-(6-nitropyridin-3-yl)piperidin-3-yl)amino)methyl)-9,10-difluoro-2,3-dihydro-7H-[1,4]oxazino[2,3,4-ij]quinolin-7-one (Step 1 in Scheme 14)

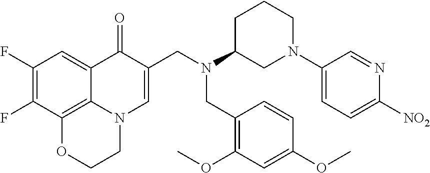 [SMILES: COc1ccc(CN(Cc2cn3c4c(c(F)c(F)cc4c2=O)OCC3)[C@H]2CCCN(c3ccc([N+](=O)[O-])nc3)C2)c(OC)c1]

To a mixture of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (90 mg, 0.2 mmol) and 2,4-dimethoxybenzaldehyde (32.7 mg, 0.2 mmol) in DCE (3 mL) was added NaBH(OAc)₃ (125.13 mg, 0.59 mmol) at room temperature under N₂ atmosphere. The resulting mixture was stirred at room temperature for 16 hours. The mixture was poured into ice-water and extracted with EA. The organic layer was washed with brine, dried over by Na₂SO₄ and concentrated under vacuum. The residue was purified by flash column (MeOH/DCM) to afford (S)-6-(((2,4-dimethoxybenzyl) (1-(6-nitropyridin-3-yl) piperidin-3-yl)amino)methyl)-9,10-difluoro-2,3-dihydro-7H-[1,4]oxazino[2,3,4-ij]quinolin-7-one 78 mg. MS(ESI): mass calcd. For C₃₁H₃₁F₂N₅O₆ 607.2 m/z found 608.2 [M+H]⁺.

### Preparation of (S)-9,10-difluoro-6-(((2-fluoro-4-(trifluoromethyl)benzyl)(1-(6-nitropyridin-3-yl)piperidin-3-yl)amino)methyl)-2,3-dihydro-7H-[1,4]oxazino[2,3,4-ij]quinolin-7-one (Step 1 in Scheme 14)

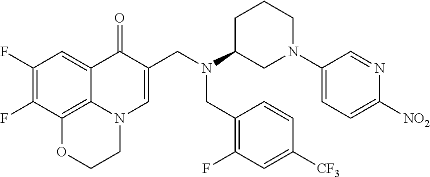 [SMILES: O=c1c(CN(Cc2ccc(C(F)(F)F)cc2F)[C@H]2CCCN(c3ccc([N+](=O)[O-])nc3)C2)cn2c3c(c(F)c(F)cc13)OCC2]

To a solution of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) in DCE (1 mL) was added 2-fluoro-4-(trifluoromethyl) benzene-1-carbaldehyde (0.09 mL, 0.66 mmol). The reaction mixture was stirred at 25° C. for 1 hours. NaBH(AcO)₃ (139 mg, 0.66 mmol) was added to the mixture in portion at 0° C. The reaction mixture was stirred at 25° C. for 2 hours. LCMS showed that (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one was consumed. The reaction mixture was poured into ice-water and extracted with AcOEt. The combined organic layer was washed with brine, dried over Na₂SO₄, filtered and concentrated. The crude product was purified by column chromatography (SiO₂, DCM:MeOH=20:1) to give (S)-9,10-difluoro-6-(((2-fluoro-4-(trifluoromethyl)benzyl) (1-(6-nitropyridin-3-yl)piperidin-3-yl)amino)methyl)-2,3-dihydro-7H-[1,4]oxazino[2,3,4-ij]quinolin-7-one (137 mg, 98.29% yield) as a yellow solid. MS (ESI): mass calcd. For C₃₀H₂₅F₆N₅O₄ 633.18 m/z found 634.2 [M+H]⁺.

### Preparation of (S)-9,10-difluoro-6-(((1-(6-nitropyridin-3-yl)piperidin-3-yl)(pyridin-2-ylmethyl)amino)methyl)-2,3-dihydro-7H-[1,4]oxazino[2,3,4-ij]quinolin-7-one (Step 1 in Scheme 14)

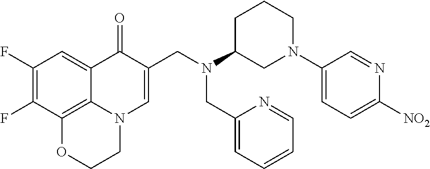 [SMILES: O=c1c(CN(Cc2ccccn2)[C@H]2CCCN(c3ccc([N+](=O)[O-])nc3)C2)cn2c3c(c(F)c(F)cc13)OCC2]

To a mixture of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) and picolinaldehyde (0.04 mL, 0.44 mmol) in DCE (3 mL) was added NaBH(OAc)₃(139.03 mg, 0.66 mmol) at room temperature under N₂ atmosphere. The resulting mixture was stirred at room temperature for 16 hours. The mixture was poured into ice-water and extracted with EA. The organic layer was washed with brine, dried over by Na₂SO₄ and concentrated under vacuum. The residue was purified by flash column (MeOH/DCM) to afford (S)-9,10-difluoro-6-(((1-(6-nitropyridin-3-yl)piperidin-3-yl) (pyridin-2-ylmethyl)amino)methyl)-2,3-dihydro-7H-[1,4]oxazino [2,3,4-ij]quinolin-7-one110 mg. MS(ESI): mass calcd. For C₂₈H₂₆F₂N₆O₄ 548.2 m/z found 549.2 [M+H]⁺.

### Preparation of (S)-6-((((6-chloropyridin-3-yl)methyl)(1-(6-nitropyridin-3-yl)piperidin-3-yl)amino)methyl)-9,10-difluoro-2,3-dihydro-7H-[1,4]oxazino[2,3,4-ij]quinolin-7-one (Step 1 in Scheme 14)

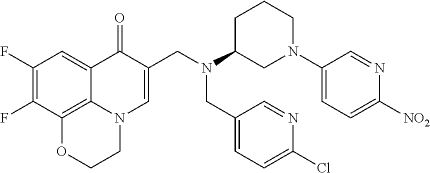 [SMILES: O=c1c(CN(Cc2ccc(Cl)nc2)[C@H]2CCCN(c3ccc([N+](=O)[O-])nc3)C2)cn2c3c(c(F)c(F)cc13)OCC2]

To a mixture of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) and 6-chloronicotinaldehyde (92.83 mg, 0.66 mmol) in DCE (3 mL) was added NaBH(OAc)₃ (139.03 mg, 0.66 mmol) at room temperature under N₂ atmosphere. The resulting mixture was stirred at room temperature for 16 hours. The mixture was poured into ice-water and extracted with EA. The organic layer was washed with brine, dried over by Na₂SO₄ and concentrated under vacuum. The residue was purified by flash column (MeOH/DCM) to afford (S)-6-((((6-chloropyridin-3-yl)methyl)(1-(6-nitropyridin-3-yl) piperidin-3-yl)amino)methyl)-9,10-difluoro-2,3-dihydro-7H-[1,4]oxazino[2,3,4-ij]quinolin-7-one 115 mg. MS(ESI): mass calcd. For C₂₈H₂₅ClF₂N₆O₄ 582.2 m/z found 583.2 [M+H]⁺.

### Preparation of (S)-9,10-difluoro-6-(((2-fluorobenzyl)(1-(6-nitropyridin-3-yl)piperidin-3-yl)amino)methyl)-2,3-dihydro-7H-[1,4]oxazino[2,3,4-ij]quinolin-7-one (Step 1 in Scheme 14)

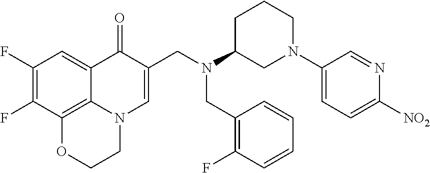 [SMILES: O=c1c(CN(Cc2ccccc2F)[C@H]2CCCN(c3ccc([N+](=O)[O-])nc3)C2)cn2c3c(c(F)c(F)cc13)OCC2]

To a solution of 9,10-difluoro-6-({[(3S)-1-(6-nitropyridin-3-yl)hexahydropyridin-3-yl]amino}methyl)-3,7-dihydro-2H-[1,4]oxazino[2,3,4-ij]quinolin-7-one (130 mg, 0.28 mmol) in DMF (2.5 mL) were added 2-(bromomethyl)-1-fluorobenzene (0.08 mL, 0.70 mmol) and K₂CO₃ (117.83 mg, 0.85 mmol). The reaction was stirred at 50° C. for 3 hours. TLC (DCM:MeOH=20:1) showed the raw material was consumed. The reaction mixture was diluted with EA (20 mL) and poured into ice-water (20 mL). The aqueous layer was extracted EA (20 mL×3). The combined organic layer was washed with brine, dried over Na₂SO₄, filtered and concentrated. The yellow residue was purified by column chromatography (SiO₂, DCM:MeOH=10:1) to give 9,10-difluoro-6-({[(2-fluorophenyl)methyl][(3S)-1-(6-nitropyridin-3-yl)hexahydropyridin-3-yl]amino}methyl)-3,7-dihydro-2H-[1,4]oxazino[2,3,4-ij]quinolin-7-one (0.15 g, 93% yield) as a yellow solid.

### Preparation of (S)-9,10-difluoro-6-(((4-fluoro-3,5-dimethylbenzyl)(1-(6-nitropyridin-3-yl)piperidin-3-yl)amino)methyl)-2,3-dihydro-7H-[1,4]oxazino[2,3,4-ij]quinolin-7-one (Step 1 in Scheme 14)

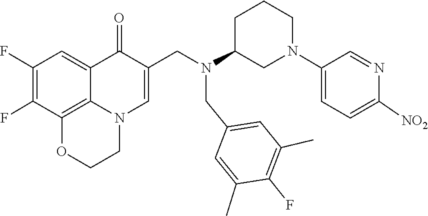 [SMILES: Cc1cc(CN(Cc2cn3c4c(c(F)c(F)cc4c2=O)OCC3)[C@H]2CCCN(c3ccc([N+](=O)[O-])nc3)C2)cc(C)c1F]

To a solution of 9,10-difluoro-6-({[(3S)-1-(6-nitropyridin-3-yl) hexahydropyridin-3-yl]amino}methyl)-3,7-dihydro-2H-[1,4]oxazino[2,3,4-ij]quinolin-7-one (130 mg, 0.28 mmol) in DMF (5 mL) was added 5-(bromomethyl)-2-fluoro-1,3-dimethylbenzene (151.96 mg, 0.70 mmol) and K₂CO₃ (117.83 mg, 0.85 mmol). The reaction was stirred at 50° C. for 3 hours. TLC (DCM:MeOH=20:1) showed the raw material was consumed. The reaction mixture was diluted with EA (20 mL) and poured into ice-water (20 mL). The aqueous layer was extracted with EA (20 mL×3). The combined organic layer was washed with brine, dried over Na₂SO₄, filtered and concentrated. The yellow residue was purified by column chromatography (SiO₂, DCM:MeOH=10:1) to give 9,10-difluoro-6-({[(4-fluoro-3,5-dimethylphenyl)methyl][(3S)-1-(6-nitropyridin-3-yl)hexahydropyridin-3-yl]amino}methyl)-3,7-dihydro-2H-[1,4]oxazino[2,3,4-ij]quinolin-7-one (0.15 g, 88% yield) as a yellow solid.

### Preparation of (S)-6-((((3-chloropyridin-4-yl)methyl)(1-(6-nitropyridin-3-yl)piperidin-3-yl)amino)methyl)-9,10-difluoro-2,3-dihydro-7H-[1,4]oxazino[2,3,4-ij]quinolin-7-one (Step 1 in Scheme 14)

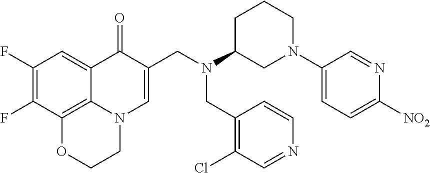 [SMILES: O=c1c(CN(Cc2ccncc2Cl)[C@H]2CCCN(c3ccc([N+](=O)[O-])nc3)C2)cn2c3c(c(F)c(F)cc13)OCC2]

To a solution of (S)-9,10-difluoro-6-(((1-(6-nitropyridin-3-yl)piperidin-3-yl) amino)methyl)-2,3-dihydro-7H-[1,4]oxazino[2,3,4-ij]quinolin-7-one (100 mg, 0.22 mmol) in DCE (1 mL) was added 3-chloroisonicotinaldehyde (186 mg, 1.31 mmol). The reaction mixture was stirred at 25° C. for 1 hour. NaBH(AcO)₃ (232 mg, 1.09 mmol) was added to the mixture in portion at 0° C. The reaction mixture was stirred at 25° C. for overnight. LCMS showed that (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=20:1) to give (S)-6-((((3-chloropyridin-4-yl)methyl)(1-(6-nitropyridin-3-yl)piperidin-3-yl)amino)methyl)-9,10-difluoro-2,3-dihydro-7H-[1,4]oxazino[2,3,4-ij]quinolin-7-one (120 mg, 94.49% yield) as a yellow solid. MS (ESI): mass calcd. For C₂₈H₂₅ClF₂N₆O₄ 582.16 m/z found 583.2 [M+H]⁺.

### Preparation of Compounds in Scheme 14 (Step 2 in Scheme 14)

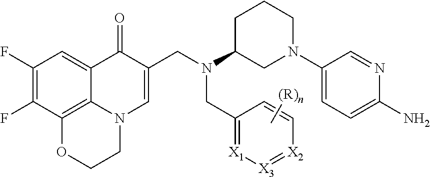

To a mixture of (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one derivatives (1 eq), C (20% wt) and FeCl₃ (0.1 eq) in EtOH (3 mL) was added NH₂·NH₂—H₂O (2 eq). The reaction mixture was stirred at 80° C. for 1 hour. LCMS showed that (S)-9,10-difluoro-6-(((1-(6-nitropyridin-3-yl)piperidin-3-yl)amino)methyl)-2,3-dihydro-7H-[1,4]oxazino[2,3,4-ij]quinolin-7-one derivatives was consumed. The reaction mixture was diluted with AcOEt, filtered and concentrated. The crude was purified to give desired product.

### Compound 70

### Preparation of (S)-6-(((1-(6-aminopyridin-3-yl)piperidin-3-yl)(pyridin-4-ylmethyl)amino)methyl)-9,10-difluoro-2,3-dihydro-7H-[1,4]oxazino[2,3,4-ij]quinolin-7-one (Step 2 in Scheme 14)

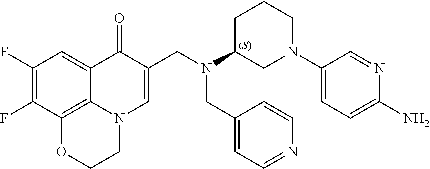

To a mixture of (S)-9,10-difluoro-6-(((1-(6-nitropyridin-3-yl)piperidin-3-yl) (pyridin-4-ylmethyl) amino) methyl)-2,3-dihydro-7H-[1,4]oxazino [2,3,4-ij]quinolin-7-one (208 mg, 0.38 mmol), C (42 mg) and FeCl₃ (6 mg, 0.038 mmol) in EtOH (3 mL) was added NH₂·NH₂·H₂O (0.04 mL, 0.76 mmol). The reaction mixture was stirred at 80° C. for 1 hour. LCMS showed that (S)-9,10-difluoro-6-(((1-(6-nitropyridin-3-yl) piperidin-3-yl)(pyridin-4-ylmethyl)amino)methyl)-2,3-dihydro-7H-[1,4]oxazino [2,3,4-ij]quinolin-7-one was consumed. The reaction mixture was diluted with AcOEt, filtered and concentrated. The crude product was purified by column chromatography (SiO₂, DCM:MeOH=15:1) to give (S)-6-(((1-(6-aminopyridin-3-yl)piperidin-3-yl) (pyridin-4-ylmethyl)amino)methyl)-9,10-difluoro-2,3-dihydro-7H-[1,4]oxazino[2,3,4-ij]quinolin-7-one (79 mg, 40.18% yield) as a white solid. ¹H NMR (DMSO-d₆, 400 MHz) δ: 8.51 (d, J=5.4 Hz, 2H), 8.05 (s, 1H), 7.69 (d, J=2.6 Hz, 1H), 7.56-7.66 (m, 1H), 7.42-7.56 (m, 2H), 7.25 (dd, J=8.9, 2.8 Hz, 1H), 6.49 (d, J=8.9 Hz, 1H), 5.84 (s, 2H), 4.67 (br t, J=4.2 Hz, 2H), 4.38-4.52 (m, 2H), 3.80-3.91 (m, 2H), 3.57-3.74 (m, 3H), 3.26 (s, 1H), 2.77-2.91 (m, 1H), 2.63-2.74 (m, 1H), 2.57-2.62 (m, 1H), 2.06 (br d, J=9.6 Hz, 1H), 1.83 (br d, J=11.1 Hz, 1H), 1.43-1.63 (m, 2H). MS (ESI): mass calcd. For C₂₈H₂₈F₂N₆O₂ 518.22 m/z found 519.2 [M+H]⁺.

### Compound 71

### Preparation of (S)-6-(((1-(6-aminopyridin-3-yl)piperidin-3-yl)((2-methylpyridin-4-yl)methyl)amino)methyl)-9,10-difluoro-2,3-dihydro-7H-[1,4]oxazino[2,3,4-ij]quinolin-7-one (Step 2 in Scheme 14)

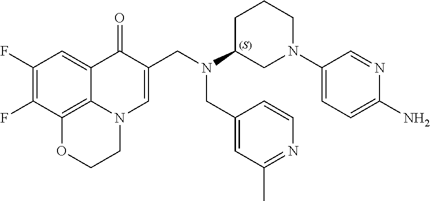

To a mixture of (S)-9,10-difluoro-6-((((2-methylpyridin-4-yl)methyl) (1-(6-nitropyridin-3-yl)piperidin-3-yl)amino)methyl)-2,3-dihydro-7H-[1,4]oxazino[2,3,4-ij]quinolin-7-one (110 mg, 0.20 mmol), C (22 mg) and FeCl₃ (3.2 mg, 0.02 mmol) in EtOH (2 mL) was added NH₂·NH₂·H₂O (0.02 mL, 0.40 mmol). The reaction mixture was stirred at 80° C. for 1 hour. LCMS showed that (S)-9,10-difluoro-6-((((2-methylpyridin-4-yl)methyl)(1-(6-nitropyridin-3-yl)piperidin-3-yl)amino)methyl)-2,3-dihydro-7H-[1,4]oxazino[2,3,4-ij]quinolin-7-one was consumed. The reaction mixture was diluted with AcOEt, filtered and concentrated. The crude product was purified by column chromatography (SiO₂, DCM:MeOH=15:1) to give (S)-6-(((1-(6-aminopyridin-3-yl)piperidin-3-yl)((2-methylpyridin-4-yl)methyl)amino)methyl)-9,10-difluoro-2,3-dihydro-7H-[1,4]oxazino [2,3,4-ij]quinolin-7-one (41 mg, 39.37% yield) as a yellow solid. ¹H NMR (DMSO-d₆, 400 MHz) δ: 8.27 (d, J=4.9 Hz, 1H), 7.93 (s, 1H), 7.59 (d, J=2.6 Hz, 1H), 7.48-7.57 (m, 1H), 7.14-7.21 (m, 3H), 6.40 (d, J=8.9 Hz, 1H), 5.26-5.68 (m, 2H), 4.57 (br t, J=4.3 Hz, 2H), 4.27-4.41 (m, 2H), 3.71 (br d, J=7.4 Hz, 2H), 3.59 (br d, J=10.5 Hz, 2H), 3.50 (br d, J=9.5 Hz, 1H), 3.14-3.23 (m, 1H), 2.67-2.82 (m, 1H), 2.56-2.64 (m, 1H), 2.39-2.45 (m, 1H), 2.36 (s, 3H), 1.97 (br d, J=9.5 Hz, 1H), 1.74 (br d, J=11.5 Hz, 1H), 1.37-1.50 (m, 2H). MS (ESI): mass calcd. For C₂₉H₃₀F₂N₆O₂ 532.24 m/z found 533.3 [M+H]⁺.

### Compound 72

### Preparation of (S)-6-(((1-(6-aminopyridin-3-yl)piperidin-3-yl)((2-methylpyridin-4-yl)methyl)amino)methyl)-9,10-difluoro-2,3-dihydro-7H-[1,4]oxazino[2,3,4-ij]quinolin-7-one (Step 2 in Scheme 14)

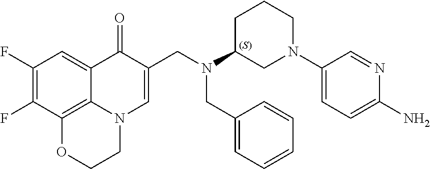

To a mixture of (S)-6-((benzyl(1-(6-nitropyridin-3-yl)piperidin-3-yl)amino) methyl)-9,10-difluoro-2,3-dihydro-7H-[1,4]oxazino[2,3,4-ij]quinolin-7-one (88 mg, 0.16 mmol), C (18 mg) and FeCl₃ (3 mg, 0.016 mmol) in EtOH (2 mL) was added NH₂·NH₂·H₂O (0.02 mL, 0.32 mmol). The reaction mixture was stirred at 80° C. for 1 hour. LCMS showed that (S)-6-((benzyl(1-(6-nitropyridin-3-yl)piperidin-3-yl) amino)methyl)-9,10-difluoro-2,3-dihydro-7H-[1,4]oxazino[2,3,4-ij]quinolin-7-one was consumed. The reaction mixture was diluted with AcOEt, filtered and concentrated. The crude product was purified by column chromatography (SiO₂, DCM:MeOH=20:1) to give (S)-6-(((1-(6-aminopyridin-3-yl)piperidin-3-yl)(benzyl) amino)methyl)-9,10-difluoro-2,3-dihydro-7H-[1,4]oxazino[2,3,4-ij]quinolin-7-one (43 mg, 51.70% yield) as a yellow solid. ¹H NMR (CHLOROFORM-d, 400 MHz) δ: 7.73 (dd, J=10.6, 7.9 Hz, 1H), 7.69 (d, J=2.6 Hz, 1H), 7.51-7.60 (m, 1H), 7.29-7.36 (m, 2H), 7.24-7.29 (m, 2H), 7.17-7.23 (m, 2H), 6.47 (d, J=8.9 Hz, 1H), 4.50 (t, J=4.6 Hz, 2H), 4.07-4.14 (m, 2H), 3.70-3.89 (m, 4H), 3.48-3.56 (m, 1H), 3.26-3.34 (m, 1H), 2.96 (ddd, J=14.3, 7.4, 3.6 Hz, 1H), 2.65-2.72 (m, 1H), 2.51 (td, J=11.8, 2.1 Hz, 1H), 2.07-2.13 (m, 1H), 1.83-1.91 (m, 1H), 1.48-1.66 (m, 2H). MS (ESI): mass calcd. For C₂₉H₂₉F₂N₅O₂ 517.23 m/z found 518.2 [M+H]⁺.

### Compound 73

### Preparation of (S)-6-(((1-(6-aminopyridin-3-yl)piperidin-3-yl)(2,4-dimethoxybenzyl)amino)methyl)-9,10-difluoro-2,3-dihydro-7H-[1,4]oxazino[2,3,4-ij]quinolin-7-one (Step 2 in Scheme 14)

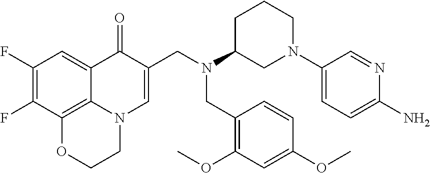 [SMILES: COc1ccc(CN(Cc2cn3c4c(c(F)c(F)cc4c2=O)OCC3)[C@H]2CCCN(c3ccc(N)nc3)C2)c(OC)c1]

A mixture of (S)-6-(((2,4-dimethoxybenzyl)(1-(6-nitropyridin-3-yl) piperidin-3-yl)amino)methyl)-9,10-difluoro-2,3-dihydro-7H-[1,4]oxazino[2,3,4-ij]quinolin-7-one (78 mg, 0.13 mmol), FeCl₃ (2.08 mg, 0.01 mmol), C (16 mg) and N₂H₄·H₂O (16.43 mg, 0.51 mmol) in EtOH (5 mL) was stirred at 80° C. for 2 hours under N₂ atmosphere. The mixture was filtered. The filtrate was collected and concentrated under vacuum. The residue was purified by flash column (MeOH/DCE) to afford (S)-6-(((1-(6-aminopyridin-3-yl)piperidin-3-yl)(2,4-dimethoxybenzyl)amino)methyl)-9,10-difluoro-2,3-dihydro-7H-[1,4]oxazino[2,3,4-ij]quinolin-7-one(9 mg). ¹H NMR (DMSO-d6) δ: 7.89 (s, 1H), 7.59 (br d, J=2.6 Hz, 1H), 7.54 (dd, J=10.8, 8.1 Hz, 1H), 7.29 (d, J=8.4 Hz, 1H), 7.13 (dd, J=8.8, 2.8 Hz, 1H), 6.48 (d, J=2.1 Hz, 1H), 6.44 (dd, J=8.3, 2.2 Hz, 1H), 6.38 (d, J=8.8 Hz, 1H), 5.31-5.44 (m, 2H), 4.53-4.62 (m, 2H), 4.33 (br d, J=4.6 Hz, 2H), 3.74 (s, 3H), 3.70 (s, 3H), 3.55-3.65 (m, 4H), 3.43 (br d, J=10.4 Hz, 1H), 3.20-3.25 (m, 1H), 2.72-2.80 (m, 1H), 2.56-2.61 (m, 1H), 2.36-2.45 (m, 1H), 1.97-1.98 (m, 1H), 1.92-2.00 (m, 1H), 1.79-1.80 (m, 1H), 1.70-1.80 (m, 1H), 1.39-1.50 (m, 1H), 1.23 (s, 3H). MS(ESI): mass calcd. For C₃₁H₃₃F₂N₅O₄ 577.3 m/z found 578.3 [M+H]⁺.

### Compound 74

### Preparation of (S)-6-(((1-(6-aminopyridin-3-yl)piperidin-3-yl)(2-fluoro-4-(trifluoromethyl)benzyl)amino)methyl)-9,10-difluoro-2,3-dihydro-7H-[1,4]oxazino[2,3,4-ij]quinolin-7-one (Step 2 in Scheme 14)

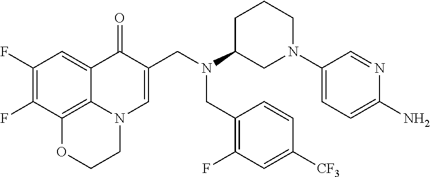 [SMILES: Nc1ccc(N2CCC[C@H](N(Cc3ccc(C(F)(F)F)cc3F)Cc3cn4c5c(c(F)c(F)cc5c3=O)OCC4)C2)cn1]

To a mixture of (S)-9,10-difluoro-6-(((2-fluoro-4-(trifluoromethyl) benzyl)(1-(6-nitropyridin-3-yl)piperidin-3-yl)amino)methyl)-2,3-dihydro-7H-[1,4]oxazino[2,3,4-ij]quinolin-7-one (137 mg, 0.22 mmol), C (28 mg) and FeCl₃ (4 mg, 0.02 mmol) in EtOH (2 mL) was added NH₂·NH₂·H₂O (0.02 mL, 0.43 mmol). The reaction mixture was stirred at 80° C. for 1 hour. LCMS showed that (S)-9,10-difluoro-6-(((2-fluoro-4-(trifluoromethyl)benzyl)(1-(6-nitropyridin-3-yl)piperidin-3-yl)amino)methyl)-2,3-dihydro-7H-[1,4]oxazino[2,3,4-ij]quinolin-7-one was consumed. The reaction mixture was diluted with AcOEt, filtered and concentrated. The crude product was purified by column chromatography (SiO₂, DCM:MeOH=20:1) to give (S)-6-(((1-(6-aminopyridin-3-yl)piperidin-3-yl)(2-fluoro-4-(trifluoromethyl) benzyl)amino)methyl)-9,10-difluoro-2,3-dihydro-7H-[1,4]oxazino[2,3,4-ij]quinolin-7-one (60 mg, 45.19% yield) as a white solid. ¹H NMR (DMSO-d₆, 400 MHz) δ: 7.90 (s, 1H), 7.77 (t, J=7.6 Hz, 1H), 7.62 (d, J=2.8 Hz, 1H), 7.46-7.53 (m, 2H), 7.38-7.45 (m, 1H), 7.15 (dd, J=8.8, 2.9 Hz, 1H), 6.38 (d, J=8.8 Hz, 1H), 5.38 (s, 2H), 4.56 (t, J=4.6 Hz, 2H), 4.30 (t, J=4.6 Hz, 2H), 3.86 (s, 2H), 3.58-3.70 (m, 2H), 3.43-3.55 (m, 1H), 3.20-3.30 (m, 1H), 2.76-2.86 (m, 1H), 2.57-2.67 (m, 1H), 2.38-2.48 (m, 1H), 1.99 (br d, J=9.1 Hz, 1H), 1.72-1.82 (m, 1H), 1.39-1.58 (m, 2H). MS (ESI): mass calcd. For C₃₀H₂₇F₆N₅O₂ 603.21 m/z found 604.3 [M+H]⁺.

### Compound 75

### Preparation of (S)-6-(((1-(6-aminopyridin-3-yl)piperidin-3-yl)(pyridin-2-ylmethyl)amino)methyl)-9,10-difluoro-2,3-dihydro-7H-[1,4]oxazino[2,3,4-ij]quinolin-7-one (Step 2 in Scheme 14)

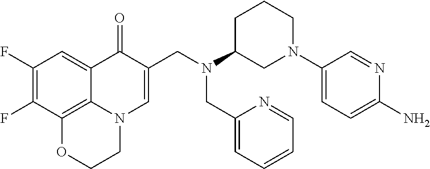 [SMILES: Nc1ccc(N2CCC[C@H](N(Cc3ccccn3)Cc3cn4c5c(c(F)c(F)cc5c3=O)OCC4)C2)cn1]

A mixture of (S)-9,10-difluoro-6-(((1-(6-nitropyridin-3-yl)piperidin-3-yl) (pyridine-2-ylmethyl)amino)methyl)-2,3-dihydro-7H-[1,4]oxazino[2,3,4-ij]quinolin-7-one (110 mg, 0.2 mmol), FeCl₃ (3.25 mg, 0.02 mmol), C (22 mg) and N₂H₄·H₂O (60 mg, 1 mmol) in EtOH (5 mL) was stirred at 80° C. for 2 hours under N₂ atmosphere. The mixture was filtered. The filtrate was collected and concentrated under vacuum. The residue was purified by flash column (MeOH/DCM) to afford (S)-6-(((1-(6-aminopyridin-3-yl)piperidin-3-yl)(pyridin-2-ylmethyl)amino)methyl)-9,10-difluoro-2,3-dihydro-7H-[1,4]oxazino[2,3,4-ij]quinolin-7-one(11 mg). ¹H NMR (DMSO-d6) δ: 8.44 (br d, J=4.3 Hz, 1H), 8.02 (s, 1H), 7.63-7.75 (m, 1H), 7.51-7.63 (m, 3H), 7.16-7.21 (m, 1H), 7.14 (dd, J=8.8, 2.8 Hz, 1H), 6.38 (d, J=8.9 Hz, 1H), 5.38 (br s, 2H), 4.58 (br t, J=4.3 Hz, 2H), 4.35 (br d, J=4.3 Hz, 2H), 3.80-3.92 (m, 2H), 3.59-3.72 (m, 1H), 3.59-3.60 (m, 1H), 3.48 (br d, J=9.8 Hz, 1H), 3.23 (br d, J=11.4 Hz, 1H), 2.71-2.88 (m, 1H), 2.58 (br t, J=10.9 Hz, 1H), 2.42 (br t, J=10.6 Hz, 1H), 1.97 (br d, J=9.5 Hz, 1H), 1.75 (br d, J=11.8 Hz, 1H), 1.36-1.54 (m, 2H). MS(ESI): mass calcd. For C₂₈H₂₈F₂N₆O₂ 518.2 m/z found 519.2 [M+H]⁺.

### Compound 76

### Preparation of (S)-6-(((1-(6-aminopyridin-3-yl)piperidin-3-yl)((6-chloropyridin-3-yl)methyl)amino)methyl)-9,10-difluoro-2,3-dihydro-7H-[1,4]oxazino[2,3,4-ij]quinolin-7-one (Step 2 in Scheme 14)

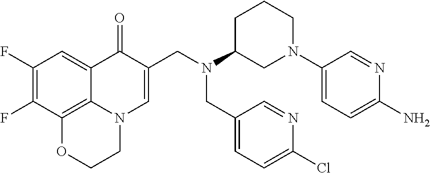 [SMILES: Nc1ccc(N2CCC[C@H](N(Cc3ccc(Cl)nc3)Cc3cn4c5c(c(F)c(F)cc5c3=O)OCC4)C2)cn1]

A mixture of (S)-6-((((6-chloropyridin-3-yl)methyl)(1-(6-nitropyridin-3-yl) piperidin-3-yl)amino)methyl)-9,10-difluoro-2,3-dihydro-7H-[1,4]oxazino[2,3,4-ij]quinolin-7-one (115 mg, 0.20 mmol), FeCl₃ (3.2 mg, 0.02 mmol), C (22 mg) and N₂H₄·H₂O (49.31 mg, 0.99 mmol) in EtOH (5 mL) was stirred at 80° C. for 2 hours under N₂ atmosphere. The mixture was filtered. The filtrate was collected and concentrated under vacuum. The residue was purified by flash column (MeOH/DCM) to afford (S)-6-(((1-(6-aminopyridin-3-yl)piperidin-3-yl)((6-chloropyridin-3-yl)methyl)amino)methyl)-9,10-difluoro-2,3-dihydro-7H-[1,4]oxazino[2,3,4-ij]quinolin-7-one (22 mg). ¹H NMR (DMSO-d6) δ: 8.38 (s, 1H), 7.88-8.00 (m, 1H), 7.79-7.88 (m, 1H), 7.63 (br d, J=2.3 Hz, 1H), 7.44-7.57 (m, 1H), 7.36 (d, J=8.1 Hz, 1H), 7.16 (br dd, J=8.8, 2.6 Hz, 1H), 6.40 (d, J=8.8 Hz, 1H), 5.40 (br s, 2H), 4.60 (br s, 2H), 4.37 (br s, 2H), 3.70-3.84 (m, 2H), 3.53-3.65 (m, 2H), 3.50 (br d, J=10.0 Hz, 1H), 3.24 (br d, J=11.3 Hz, 1H), 2.69-2.82 (m, 1H), 2.55-2.67 (m, 1H), 2.42 (br t, J=10.8 Hz, 1H), 2.04-2.14 (m, 1H), 1.90-2.04 (m, 1H), 1.65-1.86 (m, 1H), 1.36-1.58 (m, 2H). MS(ESI): mass calcd. For C₂₈H₂₇ClF₂N₆O₂ 552.2 m/z found 553.2 [M+H]⁺.

### Compound 77

### Preparation of (S)-6-(((1-(6-aminopyridin-3-yl)piperidin-3-yl)(2-fluorobenzyl)amino)methyl)-9,10-difluoro-2,3-dihydro-7H-[1,4]oxazino[2,3,4-ij]quinolin-7-one (Step 2 in Scheme 14)

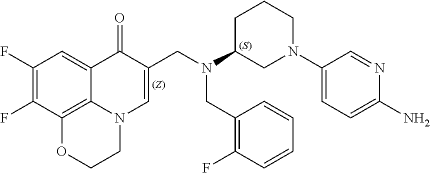

A mixture of 9,10-difluoro-6-({[(2-fluorophenyl)methyl][(3S)-1-(6-nitropyridin-3-yl)Hexahydropyridin-3-yl]amino}methyl)-3,7-dihydro-2H-[1,4]oxazino[2,3,4-ij]quinolin-7-one (190 mg, 0.34 mmol), FeCl₃ (5.45 mg, 0.03 mmol) and C (40 mg, 3.36 mmol) in EtOH (10 mL) was added N₂H₄·H₂O (188.16 mg, 3.36 mmol) at room temperature. The reaction was stirred at 80° C. for 1 hour. The mixture was filtered. The filter cake was washed with DCM. The organic layer was concentrated. The residue was purified by Pre-HPLC to give 9,10-difluoro-6-({[(3S)-1-(6-aminopyridin-3-yl)hexahydropyridin-3-yl][(2-fluorophenyl)methyl]amino}methyl)-3,7-dihydro-2H-[1,4]oxazino[2,3,4-ij]quinolin-7-one (50 mg, 0.09 mmol, 27.78% yield) as a yellow solid. ¹H NMR (DMSO-d6) δ: 7.89 (s, 1H), 7.60 (br d, J=2.3 Hz, 1H), 7.47-7.57 (m, 2H), 7.18-7.27 (m, 1H), 7.03-7.18 (m, 3H), 6.38 (d, J=8.8 Hz, 1H), 5.37 (s, 2H), 4.56 (br d, J=3.9 Hz, 2H), 4.31 (br s, 2H), 3.77 (s, 2H), 3.57-3.69 (m, 2H), 3.46 (br d, J=9.9 Hz, 1H), 3.22 (br d, J=11.1 Hz, 1H), 2.77 (br d, J=7.0 Hz, 1H), 2.55-2.65 (m, 1H), 2.50 (br s, 1H), 2.41 (br t, J=10.6 Hz, 1H), 1.97 (br d, J=8.4 Hz, 1H), 1.75 (br d, J=10.3 Hz, 1H), 1.36-1.57 (m, 2H). MS (ESI): mass calcd. For C₂₉H₂₈F₃N₅O₂ 535.22 m/z found 536.3[M+H]⁺.

### Compound 78

### Preparation of (S)-6-(((1-(6-aminopyridin-3-yl)piperidin-3-yl)(4-fluoro-3,5-dimethylbenzyl)amino)methyl)-9,10-difluoro-2,3-dihydro-7H-[1,4]oxazino[2,3,4-ij]quinolin-7-one (Step 2 in Scheme 14)

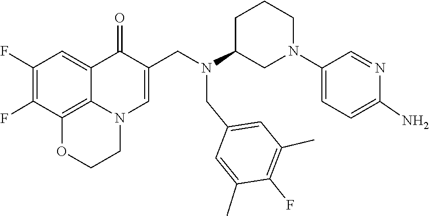 [SMILES: Cc1cc(CN(Cc2cn3c4c(c(F)c(F)cc4c2=O)OCC3)[C@H]2CCCN(c3ccc(N)nc3)C2)cc(C)c1F]

A mixture of 9,10-difluoro-6-({[(4-fluoro-3,5-dimethylphenyl)methyl][(3S)-1-(6-nitropyridin-3-yl)hexahydropyridin-3-yl]amino}methyl)-3,7-dihydro-2H-[1,4]oxazino[2,3,4-ij]quinolin-7-one (150 mg, 0.25 mmol), FeCl₃ (4.10 mg, 0.03 mmol) and C (0.02 mL, 2.53 mmol) in EtOH (10 mL) was added N₂H₄·H₂O (141.51 mg, 2.53 mmol) at room temperature. The reaction was stirred at 80° C. for 1 hour. The mixture was filtered. The filter cake was washed with DCM. The organic layer was concentrated. The residue was purified by Pre-HPLC to give 9,10-difluoro-6-({[(3S)-1-(6-aminopyridin-3-yl)hexahydropyridin-3-yl][(4-fluoro-3,5-dimethylphenyl)methyl]amino}methyl)-3,7-dihydro-2H-[1,4]oxazino[2,3,4-ij]quinolin-7-one (20 mg, 0.04 mmol, 14.29% yield) as a yellow solid. ¹H NMR (DMSO-d6) δ: 7.89 (br s, 1H), 7.60 (br s, 1H), 7.51 (br t, J=9.1 Hz, 1H), 7.13 (br d, J=7.4 Hz, 1H), 7.04 (br d, J=6.4 Hz, 2H), 6.38 (br d, J=8.6 Hz, 1H), 5.36 (br s, 2H), 4.57 (br s, 2H), 4.34 (br s, 2H), 3.52-3.68 (m, 3H), 3.52-3.68 (m, 1H), 3.47 (br d, J=9.8 Hz, 1H), 3.22 (br d, J=10.6 Hz, 1H), 2.76 (br s, 1H), 2.58 (br t, J=10.6 Hz, 1H), 2.50 (br s, 1H), 2.41 (br t, J=10.4 Hz, 1H), 2.14 (br s, 6H), 1.96 (br s, 1H), 1.73 (br d, J=9.8 Hz, 1H), 1.35-1.53 (m, 2H). MS (ESI): For C₃₁H₃₂F₃N₅O₂ 563.25 m/z found 564.3 [M+H]⁺.

### Compound 79

### Preparation of (S)-6-(((1-(6-aminopyridin-3-yl)piperidin-3-yl)((3-chloropyridin-4-yl)methyl)amino)methyl)-9,10-difluoro-2,3-dihydro-7H-[1,4]oxazino[2,3,4-ij]quinolin-7-one (Step 2 in Scheme 14)

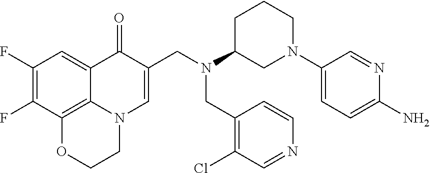 [SMILES: Nc1ccc(N2CCC[C@H](N(Cc3ccncc3Cl)Cc3cn4c5c(c(F)c(F)cc5c3=O)OCC4)C2)cn1]

To a mixture of (S)-6-((((3-chloropyridin-4-yl)methyl)(1-(6-nitropyridin-3-yl) piperidin-3-yl)amino)methyl)-9,10-difluoro-2,3-dihydro-7H-[1,4]oxazino[2,3,4-ij]quinolin-7-one (120 mg, 0.20 mmol), C (24 mg) and FeCl₃ (4 mg, 0.02 mmol) in EtOH (2 mL) was added NH₂·NH₂·H₂O (0.02 mL, 0.40 mmol). The reaction mixture was stirred at 80° C. for 1 hour. LCMS showed that (S)-6-((((3-chloropyridin-4-yl)methyl)(1-(6-nitropyridin-3-yl)piperidin-3-yl)amino)methyl)-9,10-difluoro-2,3-dihydro-7H-[1,4]oxazino[2,3,4-ij]quinolin-7-one was consumed. The reaction mixture was diluted with AcOEt. The filtered was concentrated. The crude product was purified by column chromatography (SiO₂, DCM:MeOH=20:1) to give (S)-6-(((1-(6-aminopyridin-3-yl)piperidin-3-yl)((3-chloropyridin-4-yl)methyl)amino)methyl)-9,10-difluoro-2,3-dihydro-7H-[1,4]oxazino[2,3,4-ij]quinolin-7-one (40 mg, 35.15% yield) as a white solid. ¹H NMR (DMSO-d₆, 400 MHz) δ: 8.46 (s, 1H), 8.38 (d, J=5.0 Hz, 1H), 7.97 (s, 1H), 7.70 (d, J=4.9 Hz, 1H), 7.61 (d, J=2.9 Hz, 1H), 7.53 (dd, J=10.8, 8.1 Hz, 1H), 7.13-7.20 (m, 1H), 6.38 (d, J=8.9 Hz, 1H), 5.37 (s, 2H), 4.56 (t, J=4.4 Hz, 2H), 4.31 (br d, J=4.6 Hz, 2H), 3.83-3.92 (m, 2H), 3.60-3.70 (m, 2H), 3.51 (br d, J=11.9 Hz, 1H), 3.24 (br d, J=11.6 Hz, 1H), 2.75-2.84 (m, 1H), 2.58-2.65 (m, 1H), 1.98-2.02 (m, 2H), 1.72-1.80 (m, 1H), 1.41-1.53 (m, 2H). MS (ESI): mass calcd. For C₂₈H₂₇ClF₂N₆O₂ 552.19 m/z found 553.2 [M+H]⁺.

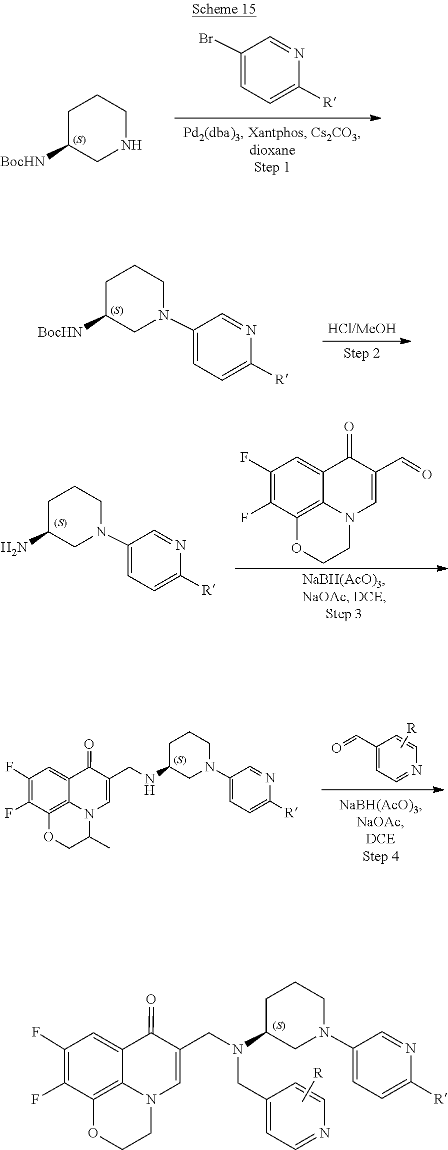

### General Procedures for Preparing Compounds in Scheme 15

### Preparation of Compounds in Scheme 15 (Step 1 in Scheme 15)

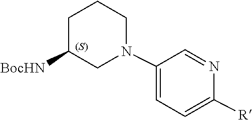

A mixture of tert-butyl N-(3-piperidyl)carbamate (22.67 mmol, 1.3 eq), 5-bromo-2-methyl (or H)-pyridine (17.44 mmol, 1 eq), Pd₂(dba)₃ (871.98 umol, 0.05 eq), Xantphos (871.98 umol, 0.05 eq) and Cs₂CO₃ (23.54 mmol, 1.35 eq) in dioxane (3 mL/mmol˜4 mL/mmol) was degassed and purged with N₂ for 3 times. Then the reaction mixture was stirred at 100° C. 115° C. for 10 hours˜12 hours under N₂ atmosphere. LCMS showed the reaction was complete. The reaction mixture was cooled to room temperature, filtered through a pad of the Celite. The filtrate was concentrated under reduced pressure. The residue was purified by flash silica gel chromatography to give the desired product.

### Preparation of tert-butyl (S)-(1-(6-methylpyridin-3-yl)piperidin-3-yl)carbamate (Step 1 in Scheme 15)

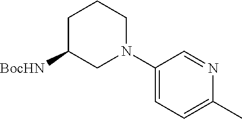 [SMILES: Cc1ccc(N2CCC[C@H](NC(=O)OC(C)(C)C)C2)cn1]

A mixture of tert-butyl (S)-piperidin-3-ylcarbamate (1 g, 4.99 mmol), 5-bromo-2-methylpyridine (0.86 g, 4.99 mmol), Pd₂(dba)₃ (0.46 g, 0.5 mmol), XantPhos (0.58 g, 1.00 mmol) and Cs₂CO₃ (3.25 g, 9.99 mmol) in dioxane was stirred at 115° C. for 16 hours under N₂ atmosphere. The resulting mixture was poured into ice-water and extracted with EA. The organic layer was washed with brine, dried over by Na₂SO₄ and concentrated under vacuum. The residue was purified by flash column (EA/PE) to afford tert-butyl (S)-(1-(6-methylpyridin-3-yl) piperidin-3-yl)carbamate (980 mg). MS(ESI): mass calcd. For C₁₆H₂₅N₃O₂ 291.2 m/z found 292.2 [M+H]⁺.

### Preparation of Compounds in Scheme 2 (Step 2 in Scheme 2)

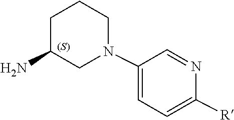

To a solution of tert-butyl N-[1-(6-methyl (or H)-3-pyridyl)-3-piperidyl]carbamate (3.43 mmol, 1 eq) in EtOAc (3 mL/mmol) or MeOH (3 mL/mmol) was added HCl/EtOAc (4 M, 6 mL/mmol) or HCl/MeOH (4 M, 4 mL/mmol). The reaction mixture was stirred at 20° C. for 12 hours. LCMS showed the reaction was complete. The mixture was concentrated under reduced pressure to obtain the desired product.

### Preparation of (S)-1-(6-methylpyridin-3-yl)piperidin-3-amine (Step 2 in Scheme 15)

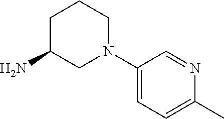

To a solution of tert-butyl (S)-(1-(6-methylpyridin-3-yl)piperidin-3-yl) carbamate (980 g, 3.36 mmol) in DCM (10 mL) was added HCl in EA (10.09 mL, 20.18 mmol) at 0° C. under N₂ atmosphere. The resulting mixture was stirred at room temperature for 2 hours. The mixture was poured into ice-water and extracted with EA. The organic layer was washed with brine, dried over by Na₂SO₄ and concentrated under vacuum. The residue was purified by flash column (MeOH/DCM) to afford (S)-1-(6-methylpyridin-3-yl)piperidin-3-amine (500 mg). MS(ESI): mass calcd. For C₁₁H₁₇N₃ 191.1 m/z found 192.1 [M+H]⁺.

### Preparation of Compounds in Scheme 15 (Step 3 in Scheme 15)

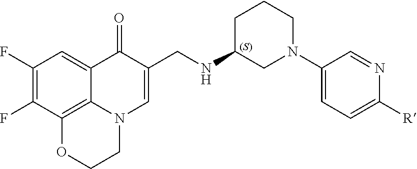

To a mixture of (3S)-1-(6-methyl (or H)-3-pyridyl)piperidin-3-amine (2.00 mmol, 1 eq, 3HCl) and 9,10-difluoro-7-oxo-2,3-dihydro-7H-[1,4]oxazino[2,3,4-ij]quinoline-6-carbaldehyde (2.00 mmol, 1 eq) in DCE (7.0 mL/mmol˜7.5 mL/mmol) was added NaOAc (2.20 mmol, 1.1 eq) at 20° C. under N₂. The mixture was stirred at 20° C. for half an hour˜5 hours and then NaBH(CN)₃ (2.99 mmol˜3.99 mmol, 1.5 eq˜2.0 eq) was added at 0° C. The mixture was stirred at 20° C. for 2 hours˜6 hours. The reaction mixture was quenched by addition water at 0° C. and then extracted with EtOAc. The combined organic layers were washed with brine, dried over Na₂SO₄, filtered and concentrated under reduced pressure to give the desired product.

### Preparation of (S)-9,10-difluoro-6-(((1-(6-methylpyridin-3-yl)piperidin-3-yl)amino)methyl)-2,3-dihydro-7H-[1,4]oxazino[2,3,4-ij]quinolin-7-one (Step 3 in Scheme 15)

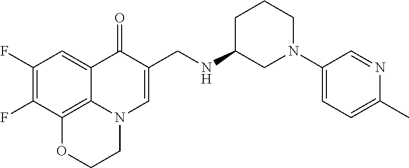 [SMILES: Cc1ccc(N2CCC[C@H](NCc3cn4c5c(c(F)c(F)cc5c3=O)OCC4)C2)cn1]

To a mixture of 9,10-difluoro-7-oxo-2,3-dihydro-7H-[1,4]oxazino[2,3,4-ij]quinoline-6-carbaldehyde (100 mg, 0.40 mmol), NaOAc (32.66 mg, 0.40 mmol) and (S)-1-(6-methylpyridin-3-yl)piperidin-3-amine (76.15 mg, 0.40 mmol) in DCM (3 mL) and MeOH (1 mL) was added NaBH(CN)₃ (75.05 mg, 1.19 mmol) at room temperature under N₂ atmosphere. The resulting mixture was stirred at room temperature for 16 hours. The mixture was poured into ice-water and extracted with EA. The organic layer was washed with brine, dried over by Na₂SO₄ and concentrated under vacuum. The residue was purified by flash column (MeOH/DCM) to afford (S)-9,10-difluoro-6-(((1-(6-methylpyridin-3-yl)piperidin-3-yl)amino)methyl)-2,3-dihydro-7H-[1,4]oxazino [2,3,4-ij]quinolin-7-one(160 mg). MS(ESI): mass calcd. For C₂₃H₂₄F₂N₄O₂ 426.2 m/z found 427.2 [M+H]⁺.

### Preparation of (S)-9,10-difluoro-6-(((1-(pyridin-3-yl)piperidin-3-yl)amino)methyl)-2,3-dihydro-7H-[1,4]oxazino[2,3,4-ij]quinolin-7-one (Step 3 in Scheme 15)

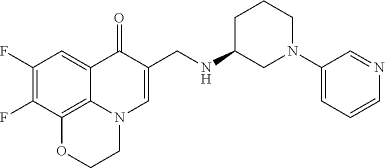 [SMILES: O=c1c(CN[C@H]2CCCN(c3cccnc3)C2)cn2c3c(c(F)c(F)cc13)OCC2]

A mixture of (3S)-1-(6-nitropyridin-3-yl)hexahydropyridin-3-amine (299.38 mg, 1.35 mmol) and 9,10-difluoro-7-oxo-2,3-dihydro[1,4]thiazino[2,3,4-ij]quinoline-6-carbaldehyde (400 mg, 1.50 mmol) in DCM (20 mL) was added AcONa (46.26 mg, 0.56 mmol) at room temperature under N₂. The reaction was stirred at room temperature for 3 hours. NaBH₃CN (39.00 mg, 0.62 mmol) was added to the reaction mixture. The mixture was stirred at room temperature overnight. The reaction mixture was adjusted to pH=5-6 with 1 N HCl and extracted with DCM (20 mL×3). The combined organic layer was washed with brine (20 mL), dried over Na₂SO₄, filtered and concentrated. The yellow residue was purified by column chromatography (DCM:MeOH=10:1) to give 9,10-difluoro-6-({[(3S)-1-(pyridin-3-yl)hexahydropyridin-3-yl]amino}methyl)-3,7-dihydro-2H-[1,4]oxazino[2,3,4-ij]quinolin-7-one (230 mg, 0.56 mmol, 100.00% yield) as a yellow solid.

### Preparation of Compounds in Scheme 15 (Step 4 in Scheme 15)

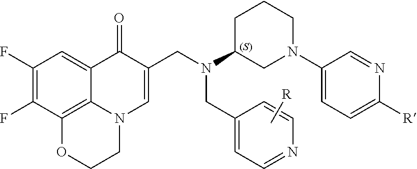

To a mixture of (S)-9,10-difluoro-6-(((1-(6-methyl (or H) 3-pyridin-yl)piperidin-3-yl)amino)methyl)-2,3-dihydro-7H-[1,4]oxazino[2,3,4-ij]quinolin-7-one (204.32 umol, 1 eq) and different aldehyde (224.75 umol, 1.1 eq) in DCE (6.5 mL/mmol˜14.7 mL/mmol) was added NaBH(OAc)₃ (306.48 umol, 1.5 eq) at 0° C. under N₂. Then the mixture was stirred at 20° C. for 10 hours˜20 hours. The reaction mixture was quenched with ice water at 0° C. and made pH=8 with sat. NaHCO₃. The mixture was extracted with DCM and i-PrOH (v:v=3:1). The combined organic layer was washed with brine (20 mL×1), dried over Na₂SO₄, filtered and concentrated under reduced pressure. The residue was purified to give the desired product.

### Compound 80

### Preparation of (S)-9,10-difluoro-6-((((2-methoxypyridin-4-yl)methyl)(1-(6-methylpyridin-3-yl)piperidin-3-yl)amino)methyl)-2,3-dihydro-7H-[1,4]oxazino[2,3,4-ij]quinolin-7-one in Scheme 15 (Step 4 in Scheme 15)

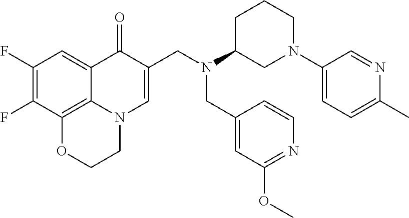 [SMILES: COc1cc(CN(Cc2cn3c4c(c(F)c(F)cc4c2=O)OCC3)[C@H]2CCCN(c3ccc(C)nc3)C2)ccn1]

To a mixture of (S)-9,10-difluoro-6-(((1-(6-methylpyridin-3-yl)piperidin-3-yl) amino)methyl)-2,3-dihydro-7H-[1,4]oxazino[2,3,4-ij]quinolin-7-one (160 mg, 0.38 mmol) and 2-methoxyisonicotinaldehyde (51.45 mg, 0.38 mmol) in DCE (5 mL) was added NaBH(OAc)₃ (238.6 mg, 1.13 mmol) at room temperature under N₂ atmosphere. The resulting mixture was stirred at room temperature for 16 hours. The mixture was poured into ice-water and extracted with EA. The organic layer was washed with brine, dried over by Na₂SO₄ and concentrated under vacuum. The residue was purified by flash column (MeOH/DCM) to afford (S)-9,10-difluoro-6-((((2-methoxypyridin-4-yl)methyl)(1-(6-methylpyridin-3-yl)piperidin-3-yl)amino)methyl)-2,3-dihydro-7H-[1,4]oxazino[2,3,4-ij]quinolin-7-one(25 mg). ¹H NMR (DMSO-d6) δ: 8.29 (br d, J=4.6 Hz, 1H), 8.14 (br s, 1H), 7.97 (s, 1H), 7.52 (br dd, J=10.0, 8.5 Hz, 1H), 7.16-7.28 (m, 3H), 7.02 (br d, J=8.4 Hz, 1H), 4.59 (br s, 2H), 4.36 (br s, 2H), 3.84 (br d, J=10.3 Hz, 1H), 3.75-3.81 (m, 1H), 3.62-3.74 (m, 1H), 3.53-3.62 (m, 2H), 2.68-2.84 (m, 2H), 2.59 (br t, J=11.3 Hz, 1H), 2.39 (s, 3H), 2.27-2.35 (m, 3H), 1.99 (br d, J=5.9 Hz, 1H), 1.75 (br d, J=10.6 Hz, 1H), 1.40-1.59 (m, 2H). MS(ESI): mass calcd. For C₃₀H₃₁F₂N₅O₃ 547.2 m/z found 548.2 [M+H]⁺.

### Compound 81

### Preparation of (S)-9,10-difluoro-6-((((2-methylpyridin-4-yl)methyl)(1-(pyridin-3-yl)piperidin-3-yl)amino)methyl)-2,3-dihydro-7H-[1,4]oxazino[2,3,4-ij]quinolin-7-one in Scheme 15 (Step 4 in Scheme 15)

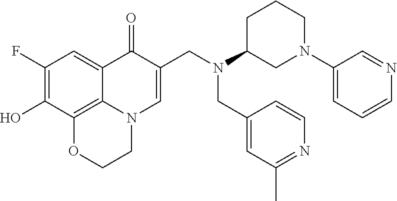 [SMILES: Cc1cc(CN(Cc2cn3c4c(c(O)c(F)cc4c2=O)OCC3)[C@H]2CCCN(c3cccnc3)C2)ccn1]

A mixture of 9,10-difluoro-6-({[(3S)-1-(pyridin-3-yl)hexahydropyridin-3-yl]amino}methyl)-3,7-dihydro-2H-[1,4]oxazino[2,3,4-ij]quinolin-7-one (240 mg, 0.58 mmol) and 2-methylpyridine-4-carbaldehyde (211 mg, 1.74 mmol) in DCE (10 mL) was added AcOH (2 drops) at room temperature under N₂. The reaction was stirred at room temperature for 3 hours. NaBH(OAc)₃ (368 mg, 1.74 mmol) was added to the mixture The mixture was stirred at room temperature overnight. The reaction mixture was adjusted to pH=7 8 with NaHCO₃ solution and extracted with DCM (20 mL×3). The combined organic layer was washed with brine (20 mL), dried over Na₂SO₄, filtered and concentrated. The yellow residue was purified by column chromatography (DCM:MeOH=10:1) to give 9,10-difluoro-6-({[(2-methylpyridin-4-yl)methyl][(3S)-1-(pyridin-3-yl)hexahydropyridin-3-yl]amino}methyl)-3,7-dihydro-2H-[1,4]oxazino [2,3,4-ij]quinolin-7-one (7.00 mg, 2.33% yield) as a white solid. ¹H NMR (DMSO-d⁶) δ: 8.28 (d, J=3.8 Hz, 2H), 7.99 (s, 1H), 7.91 (d, J=4.1 Hz, 1H), 7.53 (dd, J=10.8, 8.1 Hz, 1H), 7.21-7.31 (m, 3H), 7.15 (dd, J=8.4, 4.5 Hz, 1H), 4.58 (br t, J=4.3 Hz, 2H), 4.36 (br d, J=1.6 Hz, 2H), 3.93 (br d, J=10.8 Hz, 1H), 3.54-3.80 (m, 5H), 2.80-2.90 (m, 1H), 2.60-2.73 (m, 2H), 2.50 (br s, 3H), 2.38 (s, 3H), 2.00 (br d, J=10.3 Hz, 1H), 1.76 (br d, J=12.5 Hz, 1H), 1.39-1.58 (m, 2H). MS (ESI): mass calcd. For C₂₉H₂₉F₂N₅O₂ 517.23 m/z found 518.2[M+H]⁺.

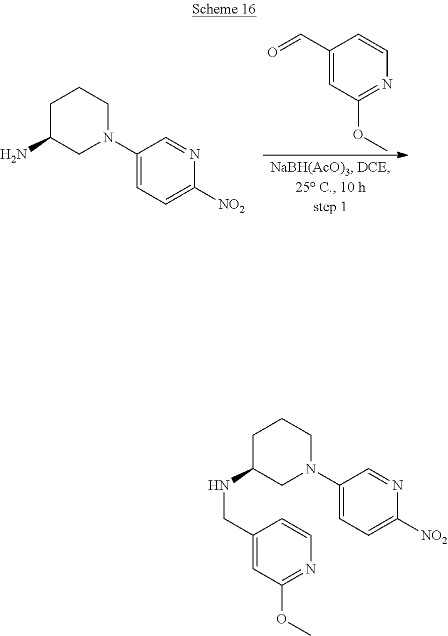

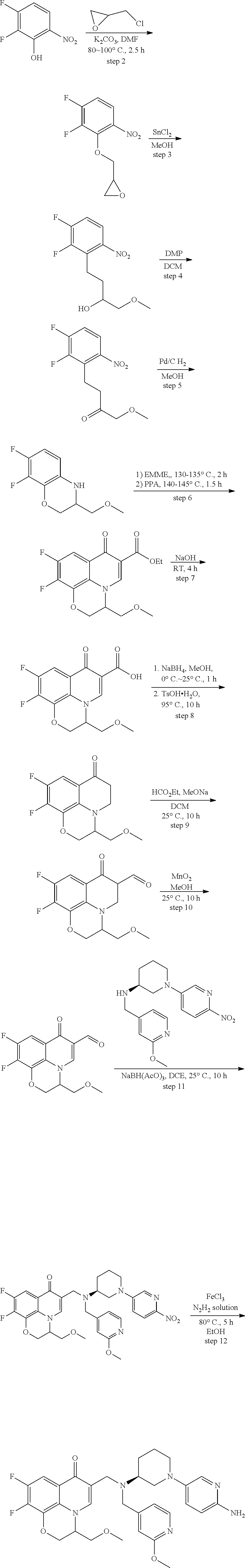

### Specific Procedures for Preparing Compounds in Scheme 16

### Preparation of (S)—N-((2-methoxypyridin-4-yl) methyl)-1-(6-nitropyridin-3-yl)piperidin-3-amine (Step 1 in Scheme 16)

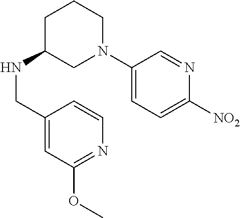 [SMILES: COc1cc(CN[C@H]2CCCN(c3ccc([N+](=O)[O-])nc3)C2)ccn1]

A mixture of (S)-1-(6-nitropyridin-3-yl)piperidin-3-amine (100 mg, 0.45 mmol) and 2-methoxyisonicotinaldehyde (61.71 mg, 0.45 mmol) in DCE (10 mL) was stirred at room temperature for 2 hours under N₂ atmosphere. NaBH(OAc)₃ (428.16 mg, 1.35 mmol) was added to the mixture. The resulting mixture was stirred at room temperature for 4 hours. The reaction mixture was poured into ice-water and extracted with EA. The organic layer was washed with brine, dried over by Na₂SO₄ and concentrated under vacuum. The residue was purified by flash column (MeOH/DCM) to afford (S)—N-((2-methoxypyridin-4-yl) methyl)-1-(6-nitropyridin-3-yl)piperidin-3-amine (120 mg). MS(ESI): mass calcd. For C₁₇H₂₁N₅O₃ 343.2 m/z found 344.2 [M+H]⁺.

### Preparation of 2-((2,3-difluoro-6-nitrophenoxy) methyl)oxirane (Step 2 in Scheme 16)

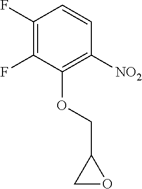 [SMILES: O=[N+]([O-])c1ccc(F)c(F)c1OCC1CO1]

A mixture of 2,3-difluoro-6-nitrophenol (13.16 mL, 114.23 mmol), 2-(chloromethyl)oxirane (10.75 mL, 137.07 mmol) and K₂CO₃ (31.57 g, 228.45 mmol) in DMF (10 mL) was stirred at 90° C. for 2.5 hours under N₂ atmosphere. The resulting mixture was poured into ice-water and extracted with EA. The organic layer was washed with brine, dried over by Na₂SO₄ and concentrated under vacuum. The residue was purified by flash column (MeOH/DCM) to afford 2-((2,3-difluoro-6-nitrophenoxy) methyl)oxirane (10.1 g). MS(ESI): mass calcd. For C₉H₇F₂NO₄ 231.0 m/z found 232.0 [M+H]⁺.

### Preparation of 1-(2,3-difluoro-6-nitrophenoxy)-3-methoxypropan-2-ol (Step 3 in Scheme 16)

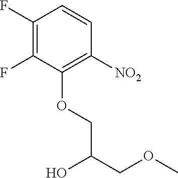 [SMILES: COCC(O)COc1c([N+](=O)[O-])ccc(F)c1F]

A mixture of 2-((2,3-difluoro-6-nitrophenoxy)methyl)oxirane (10 g, 43.26 mmol) and SnCl₂ (12.3 g, 64.89 mmol) in MeOH (50 mL) was stirred at room temperature for 2 hours under N₂ atmosphere. The resulting mixture was poured into ice-water and extracted with EA. The organic layer was washed with brine, dried over by Na₂SO₄ and concentrated under vacuum. The residue was purified by flash column (MeOH/DCM) to afford 1-(2,3-difluoro-6-nitrophenoxy)-3-methoxypropan-2-ol (4.5 g). MS(ESI): mass calcd. For C₁₀H₁₁F₂NO₅ 263.1 m/z found 264.1 [M+H]⁺.

### Preparation of 1-(2,3-difluoro-6-nitrophenoxy)-3-Methoxypropan-2-one (Step 4 in Scheme 16)

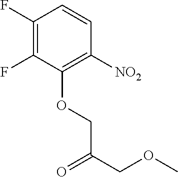 [SMILES: COCC(=O)COc1c([N+](=O)[O-])ccc(F)c1F]

A mixture of 1-(2,3-difluoro-6-nitrophenoxy)-3-methoxypropan-2-ol (4.5 g, 17.1 mmol) and DMP (14.5 g, 34.22 mmol) in DCM (50 mL) was stirred at room temperature for 16 hours under N₂ atmosphere. The resulting mixture was poured into ice-water and extracted with EA. The organic layer was washed with brine, dried over by Na₂SO₄ and concentrated under vacuum. The residue was purified by flash column (MeOH/DCM) to afford 1-(2,3-difluoro-6-nitrophenoxy)-3-Methoxypropan-2-one (1.8 g). MS(ESI): mass calcd. For C₁₀H₉F₂NO₅ 261.0 m/z found 262.0 [M+H]⁺.

### Preparation of 7,8-difluoro-3-(methoxymethyl)-3,4-dihydro-2H-benzo [b][1,4]oxazine (Step 5 in Scheme 16)

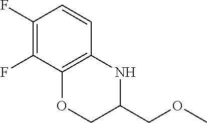

A mixture of 1-(2,3-difluoro-6-nitrophenoxy)-3-methoxypropan-2-one (500 mg, 1.91 mmol) and Pd/C (100 mg) in MeOH was stirred at room temperature for 16 hours under H₂ atmosphere. The mixture was filtered. The filtrate was collected and concentrated under vacuum to afford 7,8-difluoro-3-(methoxymethyl)-3,4-dihydro-2H-benzo [b][1,4]oxazine (380 mg). MS(ESI): mass calcd. For C₁₀H₁₁F₂NO₂ 215.1 m/z found 216.1 [M+H]⁺.

### Preparation of ethyl 9,10-difluoro-3-(methoxymethyl)-7-oxo-2,3-dihydro-7H-[1,4]oxazino [2,3,4-ij]quinoline-6-carboxylate (Step 6 in Scheme 16)

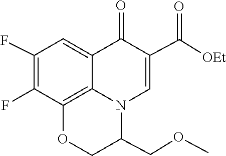 [SMILES: CCOC(=O)c1cn2c3c(c(F)c(F)cc3c1=O)OCC2COC]

A mixture of 7,8-difluoro-3-(methoxymethyl)-3,4-dihydro-2H-benzo[b][1,4]oxazine (530 mg, 2.46 mmol) and ethyl 3-ethoxy-2-(ethoxycarbonyl) prop-2-enoate (0.59 mL, 2.96 mmol) was stirred at 130° C. for 1 hour under N₂ atmosphere. Then, oxo-k5-phosphanediol (0.98 mL, 24.63 mmol) was added to the mixture. The resulting mixture was stirred at 130° C. for 2 hours under N₂ atmosphere. The resulting mixture was poured into ice-water and filtered. The filter cake was collected to afford ethyl 9,10-difluoro-3-(methoxymethyl)-7-oxo-2,3-dihydro-7H-[1,4]oxazino[2,3,4-ij]quinoline-6-carboxylate (580 mg). MS(ESI): mass calcd. For C₁₆H₁₅F₂NO₅ 339.1 m/z found 340.1 [M+H]⁺.

### Preparation of 9,10-difluoro-3-(methoxymethyl)-7-oxo-2,3-dihydro-7H-[1,4]oxazino[2,3,4-ij]quinoline-6-carboxylic Acid (Step 7 in Scheme 16)

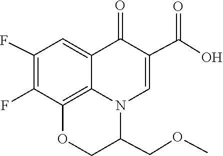 [SMILES: COCC1COc2c(F)c(F)cc3c(=O)c(C(=O)O)cn1c23]

A mixture of ethyl 9,10-difluoro-3-(methoxymethyl)-7-oxo-2,3-dihydro-7H-[1,4]oxazino[2,3,4-ij]quinoline-6-carboxylate (580 mg, 1.71 mmol) and sodium hydroxide (205.13 mg, 5.13 mmol) in THF (5 mL) was stirred at 50° C. for 1 hours under N₂ atmosphere. The resulting mixture was poured into ice-water. The pH value of the mixture was adjusted to 2 with HCl. The resulting mixture was extracted with EA and washed with brine. The organic layer was dried over by Na₂SO₄ and concentrated under vacuum to afford 9,10-difluoro-3-(methoxymethyl)-7-oxo-2,3-dihydro-7H-[1,4]oxazino[2,3,4-ij]quinoline-6-carboxylic acid (530 mg). MS(ESI): mass calcd. For Cl₄H₁₁F₂NO₅ 311.1 m/z found 312.1 [M+H]⁺.

### Preparation of 9,10-difluoro-3-(methoxymethyl)-2,3,5,6-tetrahydro-7H-[1,4]oxazino[2,3,4-ij]quinolin-7-one (Step 8 in Scheme 16)

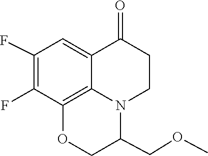 [SMILES: COCC1COc2c(F)c(F)cc3c2N1CCC3=O]

To a solution of 9,10-difluoro-3-(methoxymethyl)-7-oxo-2,3-dihydro-7H-[1,4]oxazino[2,3,4-ij]quinoline-6-carboxylic acid (530 mg, 1.70 mmol) in MeOH (3 mL) was added NaBH₄ (193.26 mg, 5.11 mmol) at 0° C. under N₂ atmosphere. The resulting mixture was stirred at room temperature for 1 hour. Then, 4-methylbenzenesulfonic acid (29.32 mg, 0.17 mmol) was added to the reaction mixture. The resulting mixture was stirred at 63° C. for 16 hours. The resulting mixture was poured into ice-water and extracted with EA. The organic layer was washed with brine, dried over by Na₂SO₄ and concentrated under vacuum. The residue was purified by flash column (EA:PE) to afford 9,10-difluoro-3-(methoxymethyl)-2,3,5,6-tetrahydro-7H-[1,4]oxazino[2,3,4-ij]quinolin-7-one (410 mg). MS(ESI): mass calcd. For C₁₃H₁₃F₂NO₃ 269.1 m/z found 270.1 [M+H]⁺.

### Preparation of 9,10-difluoro-3-(methoxymethyl)-7-oxo-2,3,6,7-tetrahydro-5H-[1,4]oxazino[2,3,4-ij]quinoline-6-carbaldehyde (Step 9 in Scheme 16)

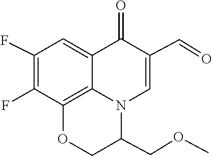 [SMILES: COCC1COc2c(F)c(F)cc3c(=O)c(C=O)cn1c23]

To a solution of 9,10-difluoro-3-(methoxymethyl)-2,3,5,6-tetrahydro-7H-[1,4]oxazino[2,3,4-ij]quinolin-7-one (410 mg, 1.52 mmol) in DCM (3 mL) was added sodium methanolate (329.04 mg, 6.09 mmol) and ethyl methanoate (0.49 mL, 6.09 mmol) at room temperature under N₂ atmosphere. The resulting mixture was stirred at room temperature for 10 minutes. The mixture was filtered. The filter cake was collected to afford 9,10-difluoro-3-(methoxymethyl)-7-oxo-2,3,6,7-tetrahydro-5H-[1,4]oxazino[2,3,4-ij]quinoline-6-carbaldehyde (380 mg). MS(ESI): mass calcd. For C₁₄H₁₃F₂NO₄ 297.1 m/z found 298.1 [M+H]⁺.

### Preparation of 9,10-difluoro-3-(methoxymethyl)-7-oxo-2,3-dihydro-7H-[1,4]oxazino[2,3,4-ij]quinoline-6-carbaldehyde (Step 10 in Scheme 16)

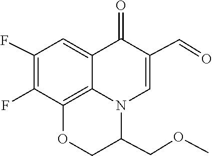 [SMILES: COCC1COc2c(F)c(F)cc3c(=O)c(C=O)cn1c23]

A mixture of 9,10-difluoro-3-(methoxymethyl)-7-oxo-2,3,6,7-tetrahydro-5H-[1,4]oxazino[2,3,4-ij]quinoline-6-carbaldehyde (380 mg, 1.28 mmol) and manganese dioxide (222.28 mg, 2.56 mmol) in MeOH (5 mL) was stirred at room temperature for 16 hours under N₂ atmosphere. The mixture was filtered. The filtrate was collected and concentrated. The residue was washed with EA and filtered. The filter cake was collected to afford 9,10-difluoro-3-(methoxymethyl)-7-oxo-2,3-dihydro-7H-[1,4]oxazino[2,3,4-ij]quinoline-6-carbaldehyde (180 mg). MS(ESI): mass calcd. For C₁₄H₁₁F₂NO₄ 295.1 m/z found 296.1 [M+H]⁺.

### Preparation of 9,10-difluoro-3-(methoxymethyl)-6-((((2-methoxypyridin-4-yl)methyl)((S)-1-(6-nitropyridin-3-yl)piperidin-3-yl)amino)methyl)-2,3-dihydro-7H-[1,4]oxazino[2,3,4-ij]quinolin-7-one (Step 11 in Scheme 16)

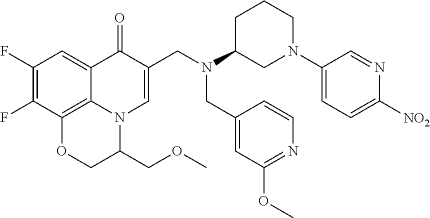 [SMILES: COCC1COc2c(F)c(F)cc3c(=O)c(CN(Cc4ccnc(OC)c4)[C@H]4CCCN(c5ccc([N+](=O)[O-])nc5)C4)cn1c23]

A mixture of 9,10-difluoro-3-(methoxymethyl)-7-oxo-2,3-dihydro-7H-[1,4]oxazino[2,3,4-ij]quinoline-6-carbaldehyde (108.33 mg, 0.37 mmol) and (S)—N-((2-methoxypyridin-4-yl)methyl)-1-(6-nitropyridin-3-yl)piperidin-3-amine (45 mg, 0.13 mmol) in DCE (1 mL) was stirred at room temperature for 2 hours under N₂ atmosphere. Then, NaBH(OAc)₃ (83.32 mg, 0.39 mmol) was added to the reaction mixture. The resulting mixture was stirred at room temperature for 4 hours. The resulting mixture was poured into ice-water, extracted with EA and washed with brine. The organic layer was dried over by Na₂SO₄ and concentrated under vacuum. The residue was purified by flash column (MeOH/DCM) to afford 9,10-difluoro-3-(methoxymethyl)-6-((((2-methoxypyridin-4-yl)methyl)((S)-1-(6-nitropyridin-3-yl)piperidin-3-yl)amino)methyl)-2,3-dihydro-7H-[1,4]oxazino[2,3,4-ij]quinolin-7-one(60 mg). MS(ESI): mass calcd. For C₃₁H₃₂F₂N₆O₆ 622.2 m/z found 623.2 [M+H]⁺.

### Compound 82

### Preparation of 6-((((S)-1-(6-aminopyridin-3-yl)piperidin-3-yl)((2-methoxypyridin-4-yl) methyl)amino)methyl)-9,10-difluoro-3-(methoxymethyl)-2,3-dihydro-7H-[1,4]oxazino[2,3,4-ij]quinolin-7-one (Step 12 in Scheme 16)

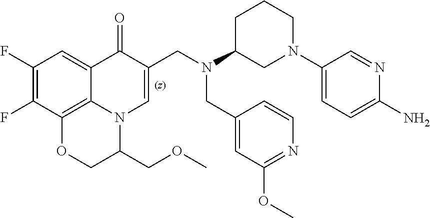 [SMILES: COCC1COc2c(F)c(F)cc3c(=O)c(CN(Cc4ccnc(OC)c4)[C@H]4CCCN(c5ccc(N)nc5)C4)cn1c23]

A mixture of 9,10-difluoro-3-(methoxymethyl)-6-((((2-methoxypyridin-4-yl) methyl)((S)-1-(6-nitropyridin-3-yl)piperidin-3-yl)amino)methyl)-2,3-dihydro-7H-[1, 4]oxazino [2,3,4-ij]quinolin-7-one (60 mg, 0.1 mmol), C (20 mg, 1.67 mmol), FeCl₃ (1.56 mg, 0.01 mmol) and N₂H₄ solution (80 mg, 0.48 mmol) in EtOH (5 mL) was stirred at 80° C. for 2 hours under N₂ atmosphere. The mixture was filtered. The filtrate was collected and concentrated. The residue was purified by flash column (MeOH/DCM) to afford 6-((((S)-1-(6-aminopyridin-3-yl)piperidin-3-yl)((2-methoxypyridin-4-yl) methyl)amino)methyl)-9,10-difluoro-3-(methoxymethyl)-2,3-dihydro-7H-[1,4]oxazino[2,3,4-ij]quinolin-7-one(5 mg). ¹H NMR (CHLOROFORM-d) δ: 8.01-8.07 (m, 1H), 7.66-7.80 (m, 3H), 7.23 (br d, J=2.8 Hz, 1H), 6.82-6.92 (m, 1H), 6.73-6.80 (m, 1H), 6.53 (dd, J=8.9, 4.1 Hz, 1H), 4.67 (br d, J=10.6 Hz, 1H), 4.27-4.37 (m, 2H), 3.87-3.91 (m, 3H), 3.73 (br d, J=4.8 Hz, 2H), 3.63-3.71 (m, 2H), 3.46-3.58 (m, 2H), 3.22 (s, 1H), 3.20-3.37 (m, 2H), 2.85-2.96 (m, 1H), 2.61-2.70 (m, 1H), 2.49 (td, J=12.0, 9.9 Hz, 1H), 1.84-1.91 (m, 1H), 1.54-1.70 (m, 2H), 1.37-1.53 (m, 2H). MS(ESI): mass calcd. For C₃₁H₃₄F₂N₆O₄ 592.3 m/z found 593.3 [M+H]⁺.

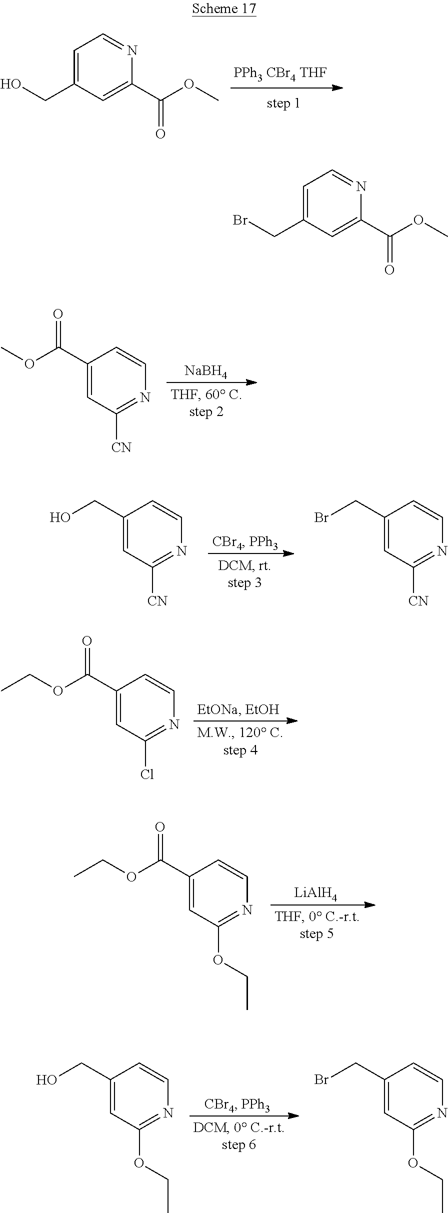

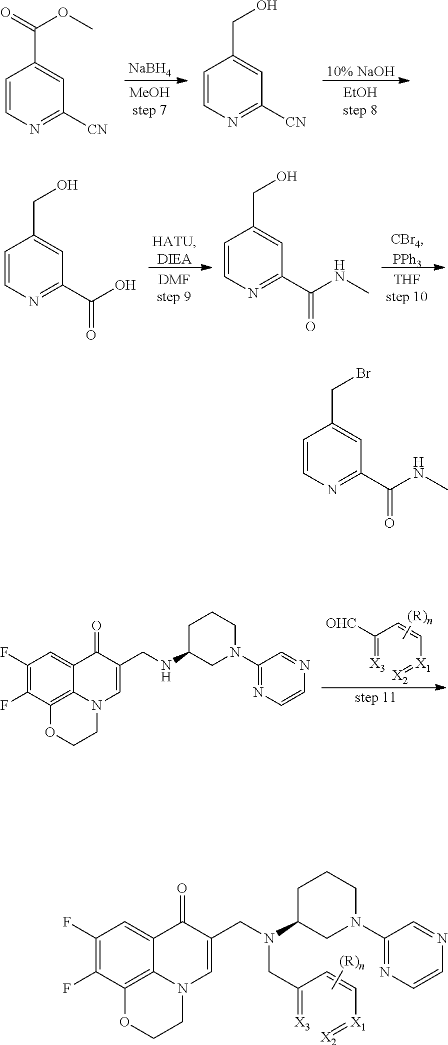

### Preparation of methyl 4-(bromomethyl)picolinate (Step 1 in Scheme 17)

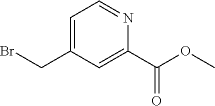

A mixture of methyl 4-(hydroxymethyl)picolinate (500 mg, 2.99 mmol), PPh₃ (1.57 g, 5.98 mmol) and CBr₄ (1.98 mg, 5.98 mmol) in THF (10 mL) was stirred at ROOM TEMPERATURE for 5 h under N₂ atmosphere. The resulting mixture was poured into ice-water and extracted with EA. The organic layer was washed with brine, dried over by Na₂SO₄ and concentrated under vacuum. The residue was purified by flash column (EA/PE) to afford methyl 4-(bromomethyl)picolinate 180 mg. MS(ESI): mass calcd. For C₈H₈BrNO₂ 229.0 m/z found 230.0 [M+H]⁺.

### Preparation of 4-(hydroxymethyl)picolinonitrile (Step 2 in Scheme 17)

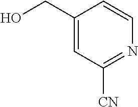

To a solution of methyl 2-cyanopyridine-4-carboxylate (2.0 g, 12.33 mmol) in tetrahydrofuran (20 mL) was added NaBH₄ (1.87 g, 49.34 mmol) in portion at 0° C. The reaction mixture was stirred at room temperature for overnight. The reaction mixture was quenched with HCl (2M, 20 mL). The aqueous layer was extracted with EA (150 mL×3). The organic layer was dried over Na₂SO₄ and concentrated. The crude product was purified by column chromatography (PE:EA=2:1) to give 4-(hydroxymethyl) picolinonitrile (1.0 g, 60.44% yield) as a yellow solid. MS m/z (ESI): 134.05 [M+H]⁺.

### Preparation of 4-(bromomethyl)picolinonitrile (Step 3 in Scheme 17)

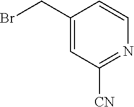

To a mixture of 4-(hydroxymethyl) pyridine-2-carbonitrile (500 mg, 3.73 mmol) and PPh₃ (1173.21 mg, 4.47 mmol) in DCM (5.0 mL) was added CBr₄ (1483.35 mg, 4.47 mmol) in portion at 0° C. under N₂ atmosphere. The reaction mixture was stirred at room temperature. for 2 hours. The reaction mixture was concentrated. The crude product was purified by column chromatography (PE:EA=2:1) to give 4-(bromomethyl)picolinonitrile (170 mg, 17.7% yield) as a yellow solid. MS m/z (ESI): 195.96 [M+H]⁺.

### Preparation of ethyl 2-ethoxyisonicotinate (Step 4 in Scheme 17)

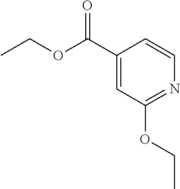

To a solution of ethyl 2-chloroisonicotinate (2.0 g, 10.78 mmol) in EtOH (15 mL) was added 2M EtONa (3.9 g, 11.85 mmol). The reaction mixture was irradiated in the microwave at 120° C. for 4 hours. LCMS showed that ethyl 2-chloroisonicotinate was consumed. The reaction mixture was cooled to 25° C. and poured into ice-water. Then the reaction mixture was adjusted to pH=6 with 2N HCl and extracted with AcOEt. The combined organic layer was washed with brine, dried over Na₂SO₄, filtered and concentrated. The crude product was purified by column chromatography (SiO₂, PE:EA=20:1) to give ethyl 2-ethoxyisonicotinate (1.552 g, 73.9% yield) as a yellow oil. MS (ESI): mass calcd. For C₁₀H₁₃NO₃ 195.09 m/z found 196.1 [M+H]⁺.

### Preparation of (2-ethoxypyridin-4-yl)methanol (Step 5 in Scheme 17)

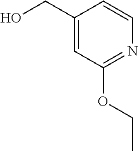

To a solution of ethyl 2-ethoxyisonicotinate (500 mg, 2.55 mmol) in THF (3 mL) was added LiAlH₄ (97 mg, 2.55 mmol) in portion at 0° C. The reaction mixture was stirred at 25° C. for 1 hour. LCMS showed that ethyl 2-ethoxyisonicotinate was consumed. The reaction mixture was poured into ice-water. Then, the reaction mixture was extracted with AcOEt. The combined organic layer was washed with brine, dried over Na₂SO₄, filtered and concentrated. The crude product was purified by column chromatography (SiO₂, DCM:MeOH=20:1) to give (2-ethoxypyridin-4-yl)methanol (390 mg, 99.4% yield) as a yellow solid. MS (ESI): mass calcd. For C₈H₁₁NO₂ 153.08 m/z found 154.1 [M+H]⁺.

### Preparation of 4-(bromomethyl)-2-ethoxypyridine (Step 6 in Scheme 17)

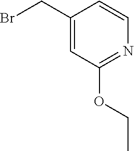

To a mixture of (2-ethoxypyridin-4-yl)methanol (350 mg, 2.28 mmol) and PPh₃ (720 mg, 2.74 mmol) in DCM (4 mL) was added CBr₄ (97 mg, 2.74 mmol) in portion at 0° C. under N₂ atmosphere. The reaction mixture was stirred at 25° C. for 2 hours. LCMS showed that (2-ethoxypyridin-4-yl)methanol was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, PE:EA=20:1) to give 4-(bromomethyl)-2-ethoxypyridine (76 mg, 15.4% yield) as a yellow solid. MS (ESI): mass calcd. For C₈H₁₀BrNO 214.99 m/z found 216.0 [M+H]⁺.

### Preparation of 4-(hydroxymethyl)picolinonitrile (Step 7 in Scheme 17)

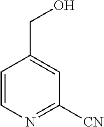

To a solution of methyl 2-cyanopyridine-4-carboxylate (3.0 g, 18.50 mmol) in MeOH (20 mL) was added NaBH₄ (1.4 g, 37.00 mmol). The reaction was stirred at room temperature for 1 hour. TLC (PE:EA=5:1) showed the reaction was completed. The reaction was quenched with 1M HCl to PH=5-7 and extracted with DCM (20 mL×3). The combined organic layer was washed with brine (20 mL), dried over Na₂SO₄, filtered and concentrated. The yellow residue was purified by column chromatography (PE:EA=5:1) to give 4-(hydroxymethyl)pyridine-2-carbonitrile (2.0 g, 14.91 mmol, 80.59% yield) as an oil.

### Preparation of 4-(hydroxymethyl)picolinic Acid (Step 8 in Scheme 17)

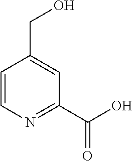 [SMILES: O=C(O)c1cc(CO)ccn1]

To a solution of 4-(hydroxymethyl)pyridine-2-carbonitrile (2.0 g, 14.91 mmol) in EtOH (10 mL) were added 10% NaOH (20 mL). The reaction was stirred at 90° C. for 2 hours. TLC (DCM:MeOH=10:1) showed the reaction was completed. The mixture was acidified with PH=3-5 and concentrated under vacuum. The residue was dissolved in DMF (20 mL), filtrated and washed with EA. The mixture was concentrated to give 4-(hydroxymethyl)pyridine-2-carboxylic acid (0.8 g, 5.22 mmol, 35.09% yield) as a yellow solid.

### Preparation of 4-(hydroxymethyl)-N-methylpicolinamide (Step 9 in Scheme 17)

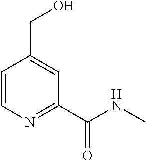

To a solution of 4-(hydroxymethyl)pyridine-2-carboxylic acid (0.60 g, 3.92 mmol) in DMF (15 mL) were added methanamine (0.80 g, 11.94 mmol), HATU (2.98 g, 7.84 mmol) and DIEA (3.25 mL, 19.59 mmol). The reaction was stirred at room temperature for 3 hours. The reaction mixture was poured into H₂O (10 mL) and extracted with EA (20 mL×3). The combined organic layer was washed with brine (20 mL), dried over Na₂SO₄, filtered and concentrated. The yellow residue was purified by column chromatography (DCM:MeOH=10:1) to give 4-(hydroxymethyl)-N-methylpyridine-2-carboxamide (0.6 g, 92% yield) as a yellow oil.

### Preparation of 4-(bromomethyl)-N-methylpicolinamide (Step 10 in Scheme 17)

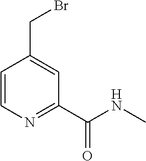

To a solution of 4-(hydroxymethyl)-N-methylpyridine-2-carboxamide (0.53 g, 3.19 mmol) in THF (15 mL) were added PPh₃ (0.84 g, 3.19 mmol) and CBr₄ (1.11 g, 3.35 mmol). The reaction was stirred at room temperature for 2 hours. TLC (PE:EA=5:1) showed the reaction was completed. The reaction mixture was poured into saturated NaCl aqueous solution (10 mL) and extracted with EA (20 mL×3). The combined organic layer was washed with brine (20 mL), dried over Na₂SO₄, filtered and concentrated. The yellow residue was purified by column chromatography (PE:EA=10:1) to give 4-(bromomethyl)-N-methylpyridine-2-carboxamide (35 mg, 0.15 mmol, 4.79% yield) as a yellow solid.

### Preparation of Compounds in Scheme 14 (Step 11 in Scheme 17)

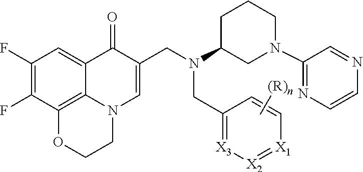

A mixture of 9,10-difluoro-6-({[(3S)-1-(6-nitropyridin-3-yl)hexahydropyridin-3-yl]amino}methyl)-3,7-dihydro-2H-[1,4]oxazino[2,3,4-ij]quinolin-7-one (1 eq) and carbaldehyde derivatives (3 eq) in DCE was added AcOH (2 drops) at room temperature under N₂ atmosphere. The reaction was stirred at room temperature. Then, NaBH(OAc)₃ (3 eq) was added to the reaction mixture. The mixture was stirred at room temperature overnight. The reaction mixture was adjusted to pH=7 to 8 with NaHCO₃ solution. The mixture was extracted with DCM. The combined organic layer was washed with brine, dried over Na₂SO₄, filtered and concentrated. The residue was purified to give the desired product.

### Compound 83

### Preparation of (S)-9,10-difluoro-6-((((2-methylpyridin-4-yl)methyl)(1-(pyrazin-2-yl)piperidin-3-yl)amino)methyl)-2,3-dihydro-7H-[1,4]oxazino[2,3,4-ij]quinolin-7-one (Step 11 in Scheme 17)

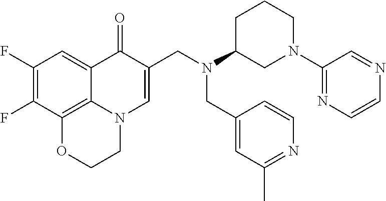 [SMILES: Cc1cc(CN(Cc2cn3c4c(c(F)c(F)cc4c2=O)OCC3)[C@H]2CCCN(c3cnccn3)C2)ccn1]

A mixture of 9,10-difluoro-6-({[(3S)-1-(6-nitropyridin-3-yl)hexahydropyridin-3-yl]amino}methyl)-3,7-dihydro-2H-[1,4]oxazino[2,3,4-ij]quinolin-7-one (80 mg, 0.17 mmol) and 2-methylpyridine-4-carbaldehyde (63.56 mg, 0.52 mmol) in DCE (5 mL) was added AcOH (2 drops) at room temperature under N₂ atmosphere. The reaction was stirred at room temperature for 3 hours. Then, NaBH(OAc)₃ (108.09 mg, 0.51 mmol) was added to the reaction mixture. The mixture was stirred at room temperature overnight. The reaction mixture was adjusted to pH=7 to 8 with NaHCO₃ solution. The mixture was extracted with DCM (20 mL×3). The combined organic layer was washed with brine (20 mL), dried over Na₂SO₄, filtered and concentrated. The yellow residue was purified by column chromatography (DCM:MeOH=10:1) to give (S)-9,10-difluoro-6-((((2-methylpyridin-4-yl)methyl)(1-(pyrazin-2-yl)piperidin-3-yl)amino)methyl)-2,3-dihydro-7H-[1,4]oxazino[2,3,4-ij]quinolin-7-one (50 mg, 0.10 mmol, 55.13% yield) was obtained as a yellow solid. ¹H NMR (DMSO-d6) δ: 8.32 (s, 1H), 8.28 (d, J=5.0 Hz, 1H), 8.02 (d, J=1.4 Hz, 1H), 7.97 (s, 1H), 7.74 (d, J=2.6 Hz, 1H), 7.53 (dd, J=10.8, 8.1 Hz, 1H), 7.20-7.25 (m, 2H), 4.49-4.61 (m, 3H), 4.23-4.39 (m, 3H), 3.56-3.82 (m, 4H), 2.99 (t, J=11.9 Hz, 1H), 2.74-2.84 (m, 1H), 2.57-2.66 (m, 1H), 2.37 (s, 3H), 2.01 (br d, J=12.1 Hz, 1H), 1.61-1.81 (m, 2H), 1.28-1.43 (m, 1H); MS (ESI): mass calcd. For C₂₈H₂₈F₂N₆O₂ 518.22 m/z found 518.3 [M+H]⁺.

### Compound 84

### Preparation of (S)-9,10-difluoro-6-((((2-methoxypyridin-4-yl)methyl)(1-(pyrazin-2-yl)piperidin-3-yl)amino)methyl)-2,3-dihydro-7H-[1,4]oxazino[2,3,4-ij]quinolin-7-one (Step 11 in Scheme 17)

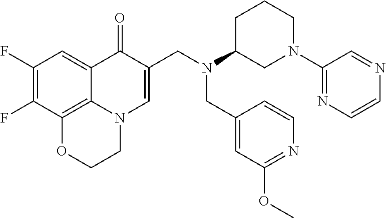 [SMILES: COc1cc(CN(Cc2cn3c4c(c(F)c(F)cc4c2=O)OCC3)[C@H]2CCCN(c3cnccn3)C2)ccn1]

To a solution of 9,10-difluoro-6-({[(3S)-1-(pyrazin-2-yl)hexahydropyridin-3-yl]amino}methyl)-3,7-dihydro-2H-[1,4]oxazino[2,3,4-ij]quinolin-7-one (60 mg, 0.15 mmol) in DCE (1 mL) was added 2-methoxypyridine-4-carbaldehyde (59.71 mg, 0.44 mmol) in portion at room temperature. The reaction mixture was stirred at room temperature. for 20 minutes. NaBH(AcO)₃ (0.02 mL, 0.44 mmol) was added to the mixture in portion at room temperature. The reaction mixture was stirred at room temperature for overnight. The reaction mixture was poured into H₂O (20 mL) and extracted with DCM (50 mL×3). The combined organic layer was washed with brine (20 mL), dried over Na₂SO₄, filtered and concentrated. The crude product was purified by Pre-HPLC (neutral condition) to give 2-methoxyisonicotinaldehyde-(S)-9,10-difluoro-6-((((2-methoxypyridin-4-yl)methyl)(1-(pyrazin-2-yl)piperidin-3-yl)amino)methyl)-2,3-dihydro-7H-[1,4]oxazino[2,3,4-ij]quinolin-7-one-(S)-9,10-difluoro-6-(((1-(pyrazin-2-yl)piperidin-3-yl)amino)methyl)-2,3-dihydro-7H-[1,4]oxazino[2,3,4-ij]quinolin-7-one (1/1/1)(30 mg, 98% yield) as a yellow solid. ¹H NMR (DMSO-d6) δ: 8.25-8.38 (m, 1H), 7.94-8.06 (m, 3H), 7.74 (d, J=2.5 Hz, 1H), 7.50 (dd, J=10.9, 8.0 Hz, 1H), 7.00 (d, J=5.1 Hz, 1H), 6.81 (s, 1H), 4.58 (br t, J=4.5 Hz, 2H), 4.46-4.54 (m, 1H), 4.22-4.44 (m, 3H), 3.63-3.83 (m, 1H), 3.62-3.86 (m, 5H), 3.52-3.62 (m, 1H), 3.50-3.60 (m, 1H), 3.33 (s, 4H), 2.96 (br t, J=11.9 Hz, 1H), 2.69-2.85 (m, 1H), 2.52-2.67 (m, 1H), 2.00 (br d, J=10.5 Hz, 1H), 1.59-1.81 (m, 2H), 1.26-1.47 (m, 1H). MS m/z (ESI): 534.22 [M+H]⁺

### Compound 85

### Preparation of (S)-9,10-difluoro-6-(((1-(pyrazin-2-yl)piperidin-3-yl)(pyrimidin-4-ylmethyl)amino)methyl)-2,3-dihydro-7H-[1,4]oxazino[2,3,4-ij]quinolin-7-one (Step 11 in Scheme 17)

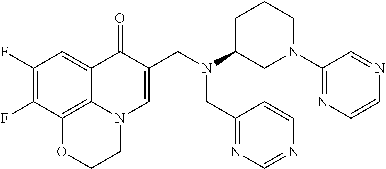 [SMILES: O=c1c(CN(Cc2ccncn2)[C@H]2CCCN(c3cnccn3)C2)cn2c3c(c(F)c(F)cc13)OCC2]

To a solution of (S)-9,10-difluoro-6-(((1-(pyrazin-2-yl)piperidin-3-yl)amino) methyl)-2,3-dihydro-7H-[1,4]oxazino[2,3,4-ij]quinolin-7-one (60 mg, 0.14 mmol) in DCE (1 mL) was added pyrimidine-4-carbaldehyde (0.04 mL, 0.44 mmol). The reaction mixture was stirred at 25° C. for 1 hour. NaBH(AcO)₃ (92 mg, 0.44 mmol) was added to the mixture in portion at 0° C. The reaction mixture was stirred at 25° C. for 2 hours. LCMS showed that (S)-9,10-difluoro-6-(((1-(pyrazin-2-yl)piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=20:1) to give (S)-9,10-difluoro-6-(((1-(pyrazin-2-yl)piperidin-3-yl)(pyrimidin-4-ylmethyl)amino)methyl)-2,3-dihydro-7H-[1,4]oxazino[2,3,4-ij]quinolin-7-one (20 mg, 27.26% yield) as a white solid. ¹H NMR (DMSO-d6) δ: 9.00 (s, 1H), 8.65 (d, J=5.1 Hz, 1H), 8.30 (s, 1H), 8.00-8.05 (m, 2H), 7.72-7.77 (m, 2H), 7.52 (dd, J=10.9, 8.0 Hz, 1H), 4.57 (br t, J=4.5 Hz, 2H), 4.50 (br d, J=12.0 Hz, 1H), 4.29-4.38 (m, 2H), 4.24 (br d, J=13.0 Hz, 1H), 3.85-3.97 (m, 2H), 3.63-3.76 (m, 2H), 2.97 (br t, J=11.8 Hz, 1H), 2.74-2.84 (m, 1H), 2.60-2.71 (m, 1H), 2.00 (br d, J=11.1 Hz, 1H), 1.72-1.80 (m, 1H), 1.57-1.71 (m, 1H), 1.32-1.45 (m, 1H). MS (ESI): mass calcd. For C₂₆H₂₅F₂N₇O₂ 505.20 m/z found 506.3 [M+H]⁺.

### Compound 86

### Preparation of (S)-9,10-difluoro-6-((((3-methoxypyridin-4-yl)methyl)(1-(pyrazin-2-yl)piperidin-3-yl)amino)methyl)-2,3-dihydro-7H-[1,4]oxazino[2,3,4-ij]quinolin-7-one (Step 11 in Scheme 17)

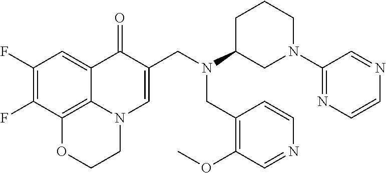 [SMILES: COc1cnccc1CN(Cc1cn2c3c(c(F)c(F)cc3c1=O)OCC2)[C@H]1CCCN(c2cnccn2)C1]

To a solution of (S)-9,10-difluoro-6-(((1-(pyrazin-2-yl)piperidin-3-yl)amino) methyl)-2,3-dihydro-7H-[1,4]oxazino[2,3,4-ij]quinolin-7-one (60 mg, 0.14 mmol) in DCE (1 mL) was added 3-methoxyisonicotinaldehyde (60 mg, 0.44 mmol). The reaction mixture was stirred at 25° C. for 1 hour. NaBH(AcO)₃ (92 mg, 0.44 mmol) was added to the mixture in portion at 0° C. The reaction mixture was stirred at 25° C. for 2 hours. LCMS showed that (S)-9,10-difluoro-6-(((1-(pyrazin-2-yl)piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=20:1) to give (S)-9,10-difluoro-6-((((3-methoxypyridin-4-yl)methyl)(1-(pyrazin-2-yl)piperidin-3-yl)amino)methyl)-2,3-dihydro-7H-[1,4]oxazino[2,3,4-ij]quinolin-7-one (20 mg, 25.78% yield) as a white solid. ¹H NMR (DMSO-d₆) δ: 8.30 (s, 1H), 8.21 (s, 1H), 8.13 (d, J=4.8 Hz, 1H), 8.02 (s, 1H), 7.95-8.00 (m, 1H), 7.73 (d, J=2.5 Hz, 1H), 7.57 (d, J=4.8 Hz, 1H), 7.49-7.56 (m, 1H), 4.55-4.61 (m, 2H), 4.46-4.54 (m, 1H), 4.30-4.41 (m, 2H), 4.21-4.28 (m, 1H), 3.86 (s, 3H), 3.71-3.83 (m, 2H), 3.57-3.71 (m, 2H), 2.97 (br t, J=11.9 Hz, 1H), 2.80 (br t, J=12.0 Hz, 1H), 2.55-2.64 (m, 1H), 2.01 (br d, J=11.9 Hz, 1H), 1.76 (br d, J=12.8 Hz, 1H), 1.66 (q, J=12.1 Hz, 1H), 1.29-1.42 (m, 1H). MS (ESI): mass calcd. For C₂₈H₂₈F₂N₆O₃ 534.22 m/z found 535.2 [M+H]⁺.

### Compound 87

### Preparation of (S)-9,10-difluoro-6-((((3-fluoropyridin-4-yl)methyl)(1-(pyrazin-2-yl)piperidin-3-yl)amino)methyl)-2,3-dihydro-7H-[1,4]oxazino[2,3,4-ij]quinolin-7-one (Step 11 in Scheme 17)

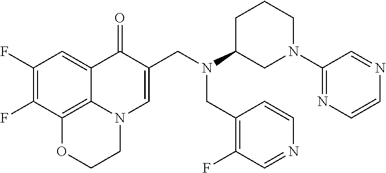 [SMILES: O=c1c(CN(Cc2ccncc2F)[C@H]2CCCN(c3cnccn3)C2)cn2c3c(c(F)c(F)cc13)OCC2]

To a solution of (S)-9,10-difluoro-6-(((1-(pyrazin-2-yl)piperidin-3-yl)amino) methyl)-2,3-dihydro-7H-[1,4]oxazino[2,3,4-ij]quinolin-7-one (60 mg, 0.15 mmol) in 1,2-dichloroethane (1 mL) was added 3-fluoropyridine-4-carbaldehyde (0.04 mL, 0.44 mmol) in portion at room temperature. The reaction mixture was stirred at room temperature for 30 minutes. Then, NaBH(AcO)₃ (92.27 mg, 0.44 mmol) was added to the mixture at 0° C. The reaction mixture was stirred at room temperature for overnight. LCMS showed that (S)-9,10-difluoro-6-(((1-(pyrazin-2-yl)piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with EA. The combined organic layer was washed with brine, dried over Na₂SO₄, filtered and concentrated. The crude product was purified by column chromatography (SiO₂, DCM:MeOH=20:1) to afford (S)-9,10-difluoro-6-((((3-fluoropyridin-4-yl)methyl) (1-(pyrazin-2-yl)piperidin-3-yl)amino)methyl)-2,3-dihydro-7H-[1,4]oxazino[2,3,4-ij]quinolin-7-one (30 mg, 0.06 mmol, 39.17% yield) as a white solid. ¹H NMR (DMSO-d₆) δ: 8.39 (d, J=1.6 Hz, 1H), 8.29-8.33 (m, 2H), 8.03 (dd, J=2.6, 1.5 Hz, 1H), 7.97 (s, 1H), 7.75 (d, J=2.6 Hz, 1H), 7.65-7.70 (m, 1H), 7.52 (dd, J=10.9, 8.0 Hz, 1H), 4.55-4.60 (m, 2H), 4.47-4.55 (m, 1H), 4.29-4.38 (m, 2H), 4.22-4.29 (m, 1H), 3.90 (s, 2H), 3.60-3.74 (m, 2H), 2.95-3.04 (m, 1H), 2.75-2.87 (m, 1H), 2.59-2.70 (m, 1H), 1.97-2.06 (m, 1H), 1.63-1.81 (m, 2H), 1.32-1.45 (m, 1H). MS m/z (ESI): 523.20 [M+H]⁺.

### Compound 88

### Preparation of methyl (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)picolinate (Step 11 in Scheme 17)

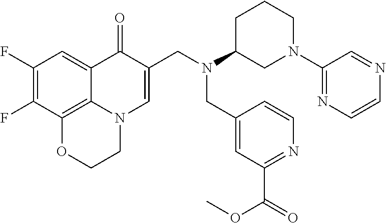 [SMILES: COC(=O)c1cc(CN(Cc2cn3c4c(c(F)c(F)cc4c2=O)OCC3)[C@H]2CCCN(c3cnccn3)C2)ccn1]

A mixture of (S)-9,10-difluoro-6-(((1-(pyrazin-2-yl)piperidin-3-yl)amino) methyl)-2,3-dihydro-7H-[1,4]oxazino[2,3,4-ij]quinolin-7-one (74.88 mg, 0.18 mmol), methyl 4-(bromomethyl)picolinate (50 mg, 0.22 mmol) and K₂CO₃ (25.03 mg, 0.18 mmol) in DMF (10 mL) was stirred at room temperature for 2 hours under N₂ atmosphere. The resulting mixture was poured into ice-water and extracted with EA. The organic layer was washed with brine, dried over by Na₂SO₄ and concentrated under vacuum. The residue was purified by flash column with MeOH/DCM to afford methyl (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)picolinate (18 mg). ¹H NMR (DMSO-d6) δ: 8.52 (d, J=4.9 Hz, 1H), 8.29-8.35 (m, 1H), 8.03 (dd, J=2.5, 1.5 Hz, 1H), 7.89-7.98 (m, 2H), 7.75 (d, J=2.6 Hz, 1H), 7.61-7.70 (m, 1H), 7.49 (dd, J=10.9, 7.9 Hz, 1H), 4.50-4.59 (m, 3H), 4.22-4.33 (m, 3H), 3.87-3.93 (m, 2H), 3.83 (s, 3H), 3.66 (q, J=14.3 Hz, 2H), 3.33 (s, 10H), 2.99 (br t, J=11.8 Hz, 1H), 2.80 (br s, 1H), 2.67 (br s, 1H), 2.08 (s, 1H), 1.98-2.06 (m, 1H), 1.75-1.83 (m, 1H), 1.63-1.74 (m, 1H), 1.34-1.45 (m, 1H), 1.21-1.27 (m, 1H). MS(ESI): mass calcd. For C₂₉H₂₈F₂N₆O₄ 562.2 m/z found 563.2 [M+H]⁺.

### Compound 89

### Preparation of (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)picolinonitrile (Step 11 in Scheme 17)

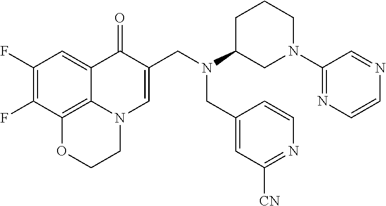 [SMILES: N#Cc1cc(CN(Cc2cn3c4c(c(F)c(F)cc4c2=O)OCC3)[C@H]2CCCN(c3cnccn3)C2)ccn1]

To a solution of 9,10-difluoro-6-({[(3S)-1-(pyrazin-2-yl)hexahydropyridin-3-yl]amino}methyl)-3,7-dihydro-2H-[1,4]oxazino[2,3,4-ij]quinolin-7-one (100 mg, 0.24 mmol) in DMF (2 mL) was added 4-(bromomethyl)pyridine-2-carbonitrile (71.49 mg, 0.36 mmol) and K₂CO₃ (50.14 mg, 0.36 mmol) in portion at room temperature. The reaction mixture was stirred at 50° C. for 2 hours. The reaction mixture was poured into water (15 mL) and extracted with EA (50 mL×3). The combined organic layer was washed with brine (15 mL), dried over Na₂SO₄, filtered and concentrated. The crude product was purified by Pre-HPLC (neutral condition) to give (S)-9,10-difluoro-6-((((2-methoxypyridin-4-yl)methyl)(1-(pyrazin-2-yl)piperidin-3-yl)amino)methyl)-2,3-dihydro-7H-[1,4]oxazino[2,3,4-ij]quinolin-7-one (25 mg, 19.52% yield) as a yellow solid. ¹H NMR (DMSO-d6) δ: 8.51-8.61 (m, 1H), 8.27-8.40 (m, 1H), 7.96-8.10 (m, 3H), 7.68-7.83 (m, 2H), 7.45-7.61 (m, 1H), 4.47-4.64 (m, 3H), 4.20-4.42 (m, 3H), 3.84-3.96 (m, 2H), 3.51-3.75 (m, 2H), 2.98 (t, J=11.9 Hz, 1H), 2.73-2.87 (m, 1H), 2.59-2.70 (m, 1H), 1.98-2.06 (m, 1H), 1.78 (br d, J=13.4 Hz, 1H), 1.58-1.73 (m, 1H), 1.29-1.51 (m, 1H). MS m/z (ESI): 529.20[M+H]⁺.

### Compound 90

### Preparation of (S)-6-((((2-ethoxypyridin-4-yl)methyl)(1-(pyrazin-2-yl)piperidin-3-yl)amino)methyl)-9,10-difluoro-2,3-dihydro-7H-[1,4]oxazino[2,3,4-ij]quinolin-7-one (Step 11 in Scheme 17)

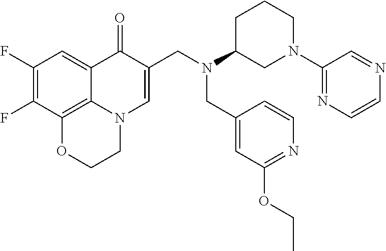 [SMILES: CCOc1cc(CN(Cc2cn3c4c(c(F)c(F)cc4c2=O)OCC3)[C@H]2CCCN(c3cnccn3)C2)ccn1]

To a mixture of (S)-9,10-difluoro-6-(((1-(pyrazin-2-yl)piperidin-3-yl)amino) methyl)-2,3-dihydro-7H-[1,4]oxazino[2,3,4-ij]quinolin-7-one (65 mg, 0.16 mmol) and K₂CO₃ (65 mg, 0.47 mmol) in DMF (1 mL) was added 4-(bromomethyl)-2-ethoxypyridine (68 mg, 0.31 mmol). The reaction mixture was stirred at 100° C. for 2 hours. LCMS showed that (S)-9,10-difluoro-6-(((1-(pyrazin-2-yl) piperidin-3-yl)amino)methyl)-2,3-dihydro-7H-[1,4]oxazino[2,3,4-ij]quinolin-7-one was consumed. The reaction mixture was poured into ice-water. Then the reaction mixture was extracted with AcOEt. The combined organic layer was washed with brine, dried over Na₂SO₄, filtered and concentrated. The crude product was purified by column chromatography (SiO₂, DCM:MeOH=20:1) to give (S)-6-((((2-ethoxypyridin-4-yl)methyl)(1-(pyrazin-2-yl)piperidin-3-yl)amino)methyl)-9,10-difluoro-2,3-dihydro-7H-[1,4]oxazino[2,3,4-ij]quinolin-7-one (7 mg, 8.1% yield) as a yellow solid. ¹H NMR (CHLOROFORM-d, 400 MHz) δ: 8.13 (s, 1H), 7.99-8.03 (m, 2H), 7.77-7.79 (m, 1H), 7.71-7.77 (m, 1H), 7.60 (s, 1H), 6.84 (d, J=4.3 Hz, 1H), 6.74 (s, 1H), 4.54 (t, J=4.7 Hz, 2H), 4.26-4.33 (m, 2H), 4.13-4.22 (m, 3H), 3.85-3.91 (m, 1H), 3.67-3.81 (m, 3H), 2.73-2.95 (m, 3H), 2.10-2.18 (m, 1H), 1.85-1.93 (m, 1H), 1.37 (t, J=7.1 Hz, 3H), 1.25 (s, 3H). MS (ESI): mass calcd. For C₂₉H₃₀F₂N₆O₃ 548.23 m/z found 549.3 [M+H]

### Compound 91

### Preparation of (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)-N-methylpicolinamide (Step 11 in Scheme 17)

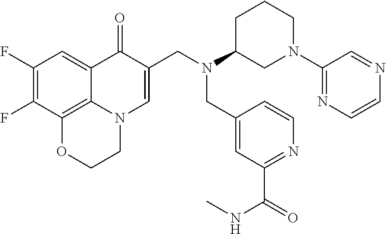 [SMILES: CNC(=O)c1cc(CN(Cc2cn3c4c(c(F)c(F)cc4c2=O)OCC3)[C@H]2CCCN(c3cnccn3)C2)ccn1]

To a solution of 9,10-difluoro-6-({[1-(pyrazin-2-yl)hexahydropyridin-3-yl]amino}methyl)-3,7-dihydro-2H-[1,4]oxazino[2,3,4-ij]quinolin-7-one (50 mg, 0.12 mmol) in DMF (2 mL) were added 4-(bromomethyl)-N-methylpyridine-2-carboxamide (30 mg, 0.13 mmol), K₂CO₃ (54.30 mg, 0.39 mmol). The reaction was stirred at 50° C. for 18 hours. The mixture was filtrated. The filtrate was concentrated to give the crude. The residue was purified by Pre-TLC (DCM:MeOH=20:1) to give (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)-N-methylpicolinamide (2.5 mg, 3.40% yield) as a yellow solid. ¹H NMR (METHANOL-d4) δ: 8.38 (d, J=5.0 Hz, 1H), 8.23 (d, J=1.3 Hz, 1H), 8.05-8.09 (m, 2H), 8.01 (s, 1H), 7.71 (d, J=2.6 Hz, 1H), 7.51-7.58 (m, 2H), 4.67 (br d, J=12.5 Hz, 1H), 4.57 (t, J=4.7 Hz, 2H), 4.37 (br s, 2H), 4.31 (br d, J=13.4 Hz, 1H), 3.96-4.02 (m, 2H), 3.78-3.89 (m, 2H), 3.67 (s, 1H), 3.10 (d, J=11.0 Hz, 1H), 2.95 (s, 3H), 2.84-2.94 (m, 2H), 2.21 (br d, J=7.5 Hz, 1H), 2.05 (br s, 1H), 1.82-1.97 (m, 2H), 1.55-1.68 (m, 2H); MS (ESI): mass calcd. For C₂₉H₂₉F₂N₇O₃ 561.23 m/z found 562.3 [M+H]⁺.

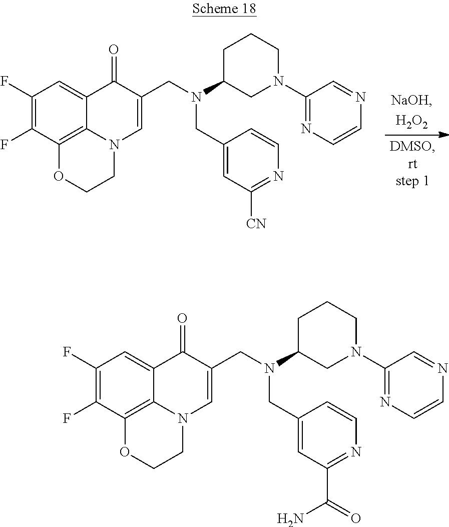 [SMILES: N#Cc1cc(CN(Cc2cn3c4c(c(F)c(F)cc4c2=O)OCC3)[C@H]2CCCN(c3cnccn3)C2)ccn1 ; NC(=O)c1cc(CN(Cc2cn3c4c(c(F)c(F)cc4c2=O)OCC3)[C@H]2CCCN(c3cnccn3)C2)ccn1]

### Compound 92

### Preparation of (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)picolinamide (Step 1 in Scheme 18)

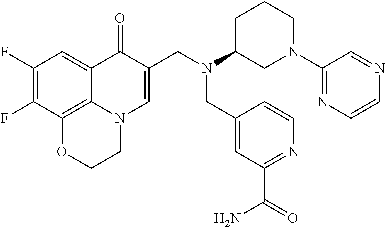 [SMILES: NC(=O)c1cc(CN(Cc2cn3c4c(c(F)c(F)cc4c2=O)OCC3)[C@H]2CCCN(c3cnccn3)C2)ccn1]

To a solution of 4-({[(9,10-difluoro-7-oxo-2,3-dihydro[1,4]oxazino [2,3,4-ij]quinolin-6-yl)methyl][(3S)-1-(pyrazin-2-yl)hexahydropyridin-3-yl]amino}methyl)pyridine-2-carbonitrile (20 mg, 0.04 mmol) in DMSO (1 mL) was added NaOH (6M) (0.019 mL) in portion at 0° C. H₂02 (0.0066 mL) was added to the mixture in portion at 0° C. The reaction mixture was stirred at room temperature for 20 minutes. H₂O (0.0436 mL, 2.42 mmol) was added to the mixture. The reaction mixture was stirred at room temperature. for 30 minutes. The reaction mixture was diluted with EA (20 mL). The organic layer was washed with brine (30 mL), dried over Na₂SO₄, filtered and concentrated. The crude product was purified by Pre-HPLC (neutral condition) to give (S)-4-((((9,10-difluoro-7-oxo-2,3-dihydro-7H-[1,4]oxazino[2,3,4-ij]quinolin-6-yl)methyl)(1-(pyrazin-2-yl)piperidin-3-yl)amino)methyl)picolinamide (1.7 mg, 8.22%) as a yellow solid. ¹H NMR (METHANOL-d4) δ: 8.36 (d, J=4.9 Hz, 1H), 8.17-8.22 (m, 1H), 8.10 (s, 1H), 8.01-8.06 (m, 1H), 7.97-8.01 (m, 1H), 7.68 (d, J=2.6 Hz, 1H), 7.46-7.58 (m, 2H), 4.60-4.68 (m, 1H), 4.49-4.59 (m, 2H), 4.35 (br s, 2H), 4.22-4.32 (m, 1H), 3.90-4.02 (m, 2H), 3.74-3.88 (m, 2H), 2.97-3.15 (m, 1H), 2.76-2.96 (m, 2H), 2.65 (s, 1H), 2.15-2.25 (m, 1H), 2.12-2.15 (m, 1H), 1.97-2.08 (m, 1H), 1.85-1.94 (m, 1H), 1.73-1.85 (m, 1H), 1.60-1.61 (m, 1H), 1.58-1.62 (m, 1H), 1.53-1.61 (m, 1H). MS m/z (ESI): 547.21 [M+H]⁺

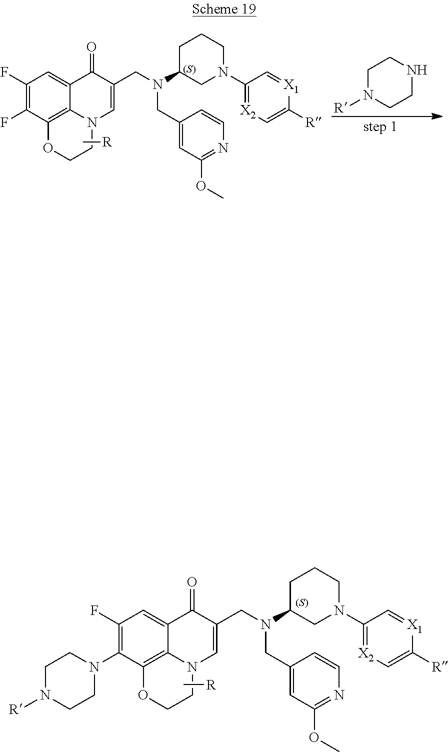

### Preparation of Compounds in Scheme 19 (Step 1 in Scheme 19)

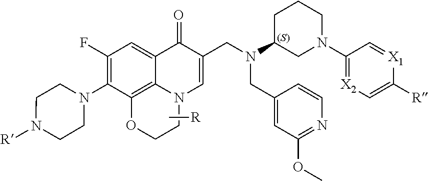

To a solution of 9,10-difluoro-6-((((2-methoxypyridin-4-yl)methyl)(piperidin-3-yl)amino)methyl)-2,3-dihydro-7H-[1,4]oxazino[2,3,4-ij]quinolin-7-one derivatives in NMP was added piperazine derivatives in portion at room temperature. The reaction mixture was stirred at 200° C. for 6 hours under microwave. The reaction mixture was added to H₂O (50 mL). The mixture was filtered and extracted with EA (40 mL×3). The combined organic layer was washed with brine (50 mL), dried over Na₂SO₄, filtered and concentrated. The residue was purified to give the desired product.

### Compound 93

### Preparation of (S)-6-(((1-(6-aminopyridin-3-yl)piperidin-3-yl)((2-methoxypyridin-4-yl)methyl)amino)methyl)-10-(4-ethylpiperazin-1-yl)-9-fluoro-2,3-dihydro-7H-[1,4]oxazino[2,3,4-ij]quinolin-7-one (Step 1 in Scheme 19)

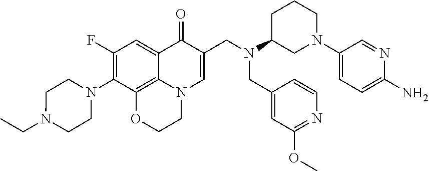 [SMILES: CCN1CCN(c2c(F)cc3c(=O)c(CN(Cc4ccnc(OC)c4)[C@H]4CCCN(c5ccc(N)nc5)C4)cn4c3c2OCC4)CC1]

To a solution of (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (50 mg, 0.09 mmol) in NMP (1 mL) was added 1-ethylpiperazine (1 mL, 7.88 mmol). The reaction mixture was stirred at 200° C. for 6 hours under microwave. LCMS showed that (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one was consumed. The reaction mixture was poured into ice-water. Then the reaction mixture was extracted with DCM/MeOH. The combined organic layer was washed with brine, dried over Na₂SO₄, filtered and concentrated. The residue was purified by column chromatography (SiO₂, DCM:MeOH=10:1) to give (S)-6-(((1-(6-aminopyridin-3-yl)piperidin-3-yl)((2-methoxypyridin-4-yl)methyl)amino)methyl)-10-(4-ethylpiperazin-1-yl)-9-fluoro-2,3-dihydro-7H-[1,4]oxazino[2,3,4-ij]quinolin-7-one (4 mg, 6.91% yield) as a yellow solid. ¹H NMR (CHLOROFORM-d, 400 MHz) δ: 8.02-8.04 (m, 1H), 7.70 (d, J=2.4 Hz, 1H), 7.60-7.65 (m, 1H), 7.49-7.52 (m, 1H), 7.21 (dd, J=8.9, 2.9 Hz, 1H), 6.82-6.86 (m, 1H), 6.75 (s, 1H), 6.46-6.50 (m, 1H), 4.43-4.47 (m, 2H), 4.09-4.13 (m, 2H), 3.89 (s, 3H), 3.80-3.86 (m, 1H), 3.72 (s, 3H), 3.63-3.69 (m, 1H), 3.52 (br dd, J=9.8, 1.4 Hz, 1H), 3.44 (br s, 4H), 3.26-3.32 (m, 1H), 2.86-2.94 (m, 1H), 2.78 (br s, 4H), 2.61-2.70 (m, 3H), 2.49 (td, J=11.8, 2.2 Hz, 1H), 2.29-2.35 (m, 1H), 2.19-2.25 (m, 1H), 1.99-2.02 (m, 1H), 1.25 (s, 3H). MS (ESI): mass calcd. For C₃₅H₄₃FN₈O₃ 642.3 m/z found 643.3 [M+H]⁺.

### Compound 94

### Preparation of (S)-6-(((1-(6-aminopyridin-3-yl)piperidin-3-yl)((2-methoxypyridin-4-yl)methyl)amino)methyl)-9-fluoro-10-(4-methylpiperazin-1-yl)-2,3-dihydro-7H-[1,4]oxazino[2,3,4-ij]quinolin-7-one (Step 1 in Scheme 19)

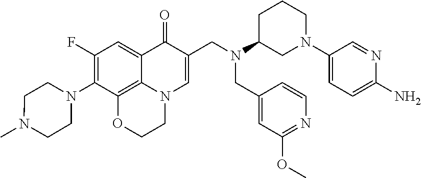 [SMILES: COc1cc(CN(Cc2cn3c4c(c(N5CCN(C)CC5)c(F)cc4c2=O)OCC3)[C@H]2CCCN(c3ccc(N)nc3)C2)ccn1]

To a solution of (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (30 mg, 0.06 mmol) in NMP (0.5 mL) was added 1-methylpiperazine (0.5 mL, 8.98 mmol). The reaction mixture was stirred at 200° C. for 6 hours under microwave. LCMS showed that (S)-6-(((1-(6-aminopyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one was consumed. The reaction mixture was poured into ice-water. Then the reaction mixture was extracted with DCM/MeOH. The combined organic layer was washed with brine, dried over Na₂SO₄, filtered and concentrated. The residue was purified by column chromatography (SiO₂, DCM:MeOH=10:1) to give (S)-6-(((1-(6-aminopyridin-3-yl)piperidin-3-yl)((2-methoxypyridin-4-yl)methyl)amino)methyl)-9-fluoro-10-(4-methylpiperazin-1-yl)-2,3-dihydro-7H-[1,4]oxazino[2,3,4-ij]quinolin-7-one (3 mg, 5.23% yield) as a yellow solid. ¹H NMR (METHANOL-d₄, 400 MHz) δ: 7.88-7.99 (m, 3H), 7.50 (d, J=12.0 Hz, 1H), 7.36 (br s, 1H), 6.95-7.02 (m, 2H), 6.79 (br s, 1H), 4.51-4.56 (m, 2H), 4.31 (br d, J=4.4 Hz, 2H), 3.72-3.81 (m, 4H), 3.58 (br s, 4H), 3.38 (br d, J=11.6 Hz, 2H), 3.34 (dt, J=3.3, 1.6 Hz, 1H), 2.94-3.02 (m, 4H), 2.15-2.31 (m, 2H), 1.95-2.08 (m, 2H), 1.55-1.76 (m, 2H), 1.25-1.37 (m, 6H). MS (ESI): mass calcd. For C₃₄H₄₁FN₈O₃ 628.3 m/z found 629.3 [M+H]⁺.

### Compound 95

### Preparation of (R)-9-fluoro-10-(4-methylpiperazin-1-yl)-6-((((5-methylpyridin-3-yl)methyl)(1-(pyrazin-2-yl)piperidin-3-yl)amino)methyl)-2,3-dihydro-7H-[1,4]oxazino[2,3,4-ij]quinolin-7-one (Step 1 in Scheme 19)

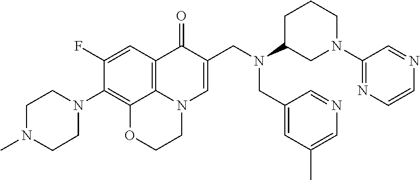

To a solution of 9,10-difluoro-6-({[(5-methylpyridin-3-yl)methyl][1-(pyrazin-2-yl)hexahydropyridin-3-yl]amino}methyl)-3,7-dihydro-2H-[1,4]oxazino[2,3,4-ij]quinolin-7-one (100 mg, 0.19 mmol) in NMP (0.5 mL) was added 1-methylpiperazine (0.5 mL, 4.49 mmol) under N₂ atmosphere. The reaction mixture was stirred at 200° C. for 6 hours under microwave. The mixture was purified by Pre-HPLC (neutral) to give 9-fluoro-10-(4-methylpiperazin-1-yl)-6-({[(5-methylpyridin-3-yl)methyl][1-(pyrazin-2-yl)hexahydropyridin-3-yl]amino}methyl)-3,7-dihydro-2H-[1,4]oxazino[2,3,4-ij]quinolin-7-one (5 mg, 0.01 mmol, 4.33% yield). ¹H NMR (METHANOL-d4) δ: 8.11-8.23 (m, 2H), 8.03 (dd, J=2.5, 1.4 Hz, 1H), 7.82 (s, 1H), 7.68 (d, J=2.6 Hz, 1H), 7.47 (d, J=12.4 Hz, 1H), 7.20-7.27 (m, 2H), 4.48 (t, J=4.5 Hz, 2H), 4.25 (br d, J=2.3 Hz, 2H), 3.84 (d, J=3.0 Hz, 2H), 3.44 (br t, J=4.3 Hz, 4H), 3.31 (dt, J=3.3, 1.6 Hz, 2H), 2.98 (br s, 4H), 2.65 (s, 3H), 2.33 (s, 3H). MS (ESI): mass calcd. For C₃₃H₃₉FN₈O₂ 598.32 m/z found 599.3[M+H]⁺.

### Compound 96

### Preparation of 6-((((S)-1-(6-aminopyridin-3-yl)piperidin-3-yl)((2-methoxypyridin-4-yl)methyl)amino)methyl)-9-fluoro-3-isopropyl-10-(4-methylpiperazin-1-yl)-2,3-dihydro-7H-[1,4]oxazino[2,3,4-ij]quinolin-7-one (Step 1 in Scheme 19)

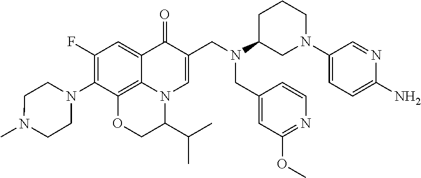 [SMILES: COc1cc(CN(Cc2cn3c4c(c(N5CCN(C)CC5)c(F)cc4c2=O)OCC3C(C)C)[C@H]2CCCN(c3ccc(N)nc3)C2)ccn1]

To a solution of 9,10-difluoro-6-({[(3S)-1-(6-aminopyridin-3-yl)hexahydropyridin-3-yl][(2-methoxypyridin-4-yl)methyl]amino}methyl)-3-(prop-2-yl)-3,7-dihydro-2H-[1,4]oxazino[2,3,4-ij]quinolin-7-one (360 mg, 0.61 mmol) in NMP (1.5 mL) was added 1-methylpiperazine (1.5 mL, 13.48 mmol) in portion at room temperature. The reaction mixture was stirred at 200° C. for 6 hours under microwave. The reaction mixture was added to H₂O (50 mL). The mixture was filtered and extracted with EA (40 mL×3). The combined organic layer was washed with brine (50 mL), dried over Na₂SO₄, filtered and concentrated. The crude product was purified by Pre-HPLC (neutral condition) to give 6-((((S)-1-(6-aminopyridin-3-yl)piperidin-3-yl)((2-methoxypyridin-4-yl)methyl)amino)methyl)-9-fluoro-3-isopropyl-10-(4-methylpiperazin-1-yl)-2,3-dihydro-7H-[1,4]oxazino[2,3,4-ij]quinolin-7-one (160 mg, 85.93% yield) as a yellow solid. ¹H NMR (DMSO-d6) δ: 8.01 (d, J=5.3 Hz, 1H), 7.89 (d, J=8.5 Hz, 1H), 7.60 (t, J=3.0 Hz, 1H), 7.34 (d, J=12.6 Hz, 1H), 7.11-7.16 (m, 1H), 6.99-7.03 (m, 1H), 6.83 (s, 1H), 6.37 (dd, J=8.8, 4.0 Hz, 1H), 5.38 (br s, 2H), 4.77 (br d, J=11.9 Hz, 1H), 4.15-4.23 (m, 2H), 3.78 (s, 3H), 3.71-3.76 (m, 2H), 3.61-3.69 (m, 1H), 3.43-3.54 (m, 2H), 3.10-3.28 (m, 6H), 2.68-2.79 (m, 1H), 2.53-2.63 (m, 1H), 2.41 (br s, 5H), 2.22 (s, 3H), 1.95 (br dd, J=12.5, 6.1 Hz, 2H), 1.74 (br s, 1H), 1.35-1.51 (m, 2H), 0.99 (dd, J=12.3, 6.7 Hz, 3H), 0.72 (dd, J=10.6, 6.9 Hz, 3H). MS (ESI): mass calcd. For C₃₇H₄₇FN₅O₃ 670.8 m/z found 671.38 [M+H]⁺.

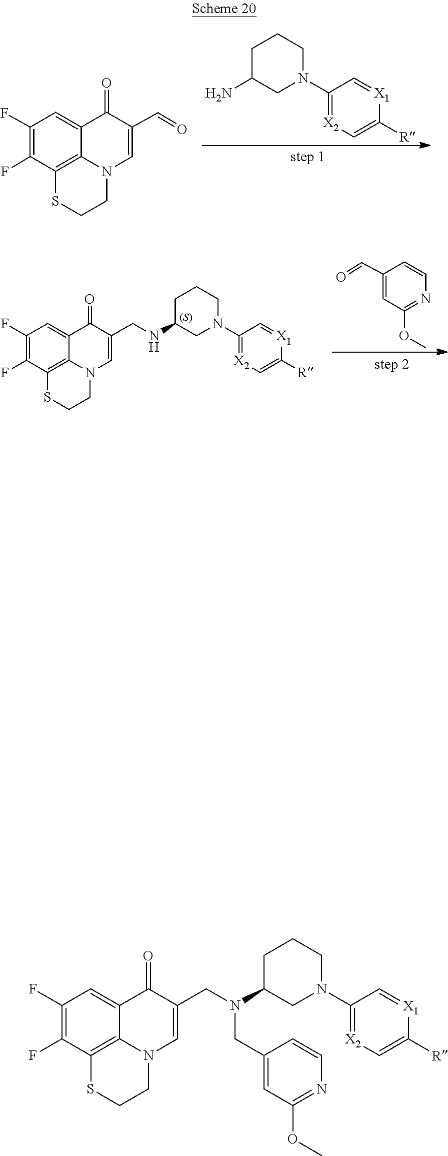

### Preparation of Compounds in Scheme 20 (Step 1 in Scheme 20)

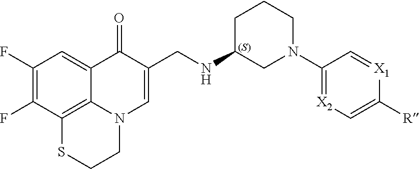

To a solution of 9,10-difluoro-7-oxo-2,3-dihydro[1,4]thiazino[2,3,4-ij]quinoline-6-carbaldehyde (1 eq) in MeOH (1 mL) and DCM (1 mL) was added piperidin-3-amine derivatives (1 eq) and NaOAc (1 eq) in portion at room temperature. The reaction mixture was stirred at room temperature for 2 hours. NaBH₃CN (4 eq) was added to the reaction mixture in portion. The reaction mixture was stirred at room temperature for 1 hour. The reaction mixture was poured into H₂O (30 mL) and extracted with DCM (50 mL×3). The combined organic layer was washed with brine (50 mL), dried over Na₂SO₄, filtered and concentrated. The residue was purified to give the desired product.

### Preparation of (S)-9,10-difluoro-6-(((1-(pyrazin-2-yl)piperidin-3-yl)amino)methyl)-2,3-dihydro-7H-[1,4]thiazino[2,3,4-ij]quinolin-7-one (Step 1 in Scheme 20)

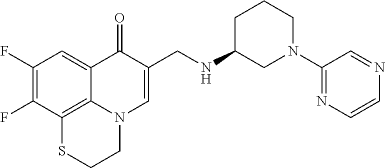 [SMILES: O=c1c(CN[C@H]2CCCN(c3cnccn3)C2)cn2c3c(c(F)c(F)cc13)SCC2]

To a solution of 9,10-difluoro-7-oxo-2,3-dihydro[1,4]thiazino[2,3,4-ij]quinoline-6-carbaldehyde (100 mg, 0.37 mmol) in MeOH (1 mL) and DCM (1 mL) was added 1-(pyrazin-2-yl)hexahydropyridin-3-amine (66.69 mg, 0.37 mmol) and NaOAc (30.69 mg, 0.37 mmol) in portion at room temperature. The reaction mixture was stirred at room temperature for 2 hours. NaBH₃CN (0.04 mL, 1.12 mmol) was added to the reaction mixture in portion. The reaction mixture was stirred at room temperature for 1 hour. The reaction mixture was poured into H₂O (30 mL) and extracted with DCM (50 mL×3). The combined organic layer was washed with brine (50 mL), dried over Na₂SO₄, filtered and concentrated to give crude product (S)-9,10-difluoro-6-(((1-(pyrazin-2-yl)piperidin-3-yl)amino)methyl)-2,3-dihydro-7H-[1,4]thiazino[2,3,4-ij]quinolin-7-one (160 mg, 99.56% yield) as a yellow solid. MS (ESI): mass calcd. For C₂₁H₂₁F₂N₅OS 429.4 m/z found 430.14 [M+H]⁺.

### Preparation of (S)-6-(((1-(6-((cyclopropylmethyl)amino)pyridin-3-yl)piperidin-3-yl)amino)methyl)-9,10-difluoro-2,3-dihydro-7H-[1,4]thiazino[2,3,4-ij]quinolin-7-one (Step 1 in Scheme 20)

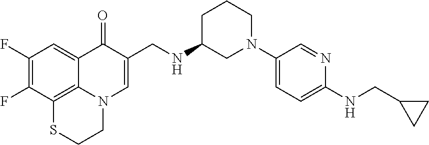 [SMILES: O=c1c(CN[C@H]2CCCN(c3ccc(NCC4CC4)nc3)C2)cn2c3c(c(F)c(F)cc13)SCC2]

To a solution of 9,10-difluoro-7-oxo-2,3-dihydro[1,4]thiazino[2,3,4-ij]quinoline-6-carbaldehyde (100 mg, 0.37 mmol) in MeOH (1 mL) and DCM (1 mL) was added 1-{6-[(cyclopropylmethyl)amino]pyridin-3-yl}hexahydropyridin-3-amine (92.18 mg, 0.37 mmol) and NaOAc (30.69 mg, 0.37 mmol) in portion at room temperature. The reaction mixture was stirred at room temperature for 2 hours. NaBH₃CN (70.54 mg, 1.12 mmol) was added to the reaction mixture in portion. The reaction mixture was stirred at room temperature. for 1 hour. The reaction mixture was poured into H₂O (30 mL) and extracted with DCM (50 mL×3). The combined organic layer was washed with brine (50 mL), dried over Na₂SO₄, filtered and concentrated. The crude product was purified by column chromatography (SiO₂, DCM(NH₄OH):MeOH=5:1) to give 4-(hydroxymethyl) picolinonitrile (160 mg, 85.93% yield) as a yellow solid. MS (ESI): mass calcd. For C₂₆H₂₉F₂N₅OS 497.6 m/z found 498.21[M+H]⁺.

### Preparation of Compounds in Scheme 20 (Step 2 in Scheme 20)

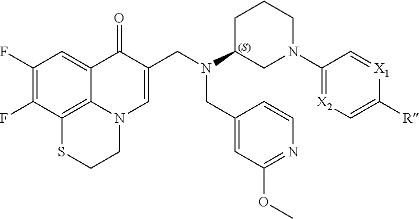

To a solution of (S)-9,10-difluoro-6-((piperidin-3-ylamino)methyl)-2,3-dihydro-7H-[1,4]thiazino[2,3,4-ij]quinolin-7-one derivatives (1 eq) in DCE (2 mL) was added 2-methylpyridine-4-carbaldehyde (3 eq) in portion at room temperature. The reaction mixture was stirred at room temperature for 30 minutes. NaBH(AcO)₃ (2 eq) was added to the reaction mixture in portion. The reaction mixture was stirred at room temperature for overnight. The reaction mixture was poured into H₂O (20 mL) and extracted with DCM (50 mL×3). The combined organic layer was washed with brine (20 mL), dried over Na₂SO₄, filtered and concentrated. The residue was purified to give the desired product.

### Compound 97

### Preparation of (S)-9,10-difluoro-6-((((2-methylpyridin-4-yl)methyl)(1-(pyrazin-2-yl)piperidin-3-yl)amino)methyl)-2,3-dihydro-7H-[1,4]thiazino[2,3,4-ij]quinolin-7-one (Step 2 in Scheme 20)

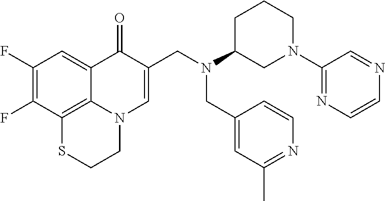 [SMILES: Cc1cc(CN(Cc2cn3c4c(c(F)c(F)cc4c2=O)SCC3)[C@H]2CCCN(c3cnccn3)C2)ccn1]

To a solution of 9,10-difluoro-6-({[(3S)-1-(pyrazin-2-yl)hexahydropyridin-3-yl]amino}methyl)-3,7-dihydro-2H-[1,4]thiazino[2,3,4-ij]quinolin-7-one (100 mg, 0.23 mmol) in DCE (2 mL) was added 2-methylpyridine-4-carbaldehyde (84.62 mg, 0.70 mmol) in portion at room temperature. The reaction mixture was stirred at room temperature for 30 minutes. NaBH(AcO)₃ (0.02 mL, 0.44 mmol) was added to the reaction mixture in portion. The reaction mixture was stirred at room temperature for overnight. The reaction mixture was poured into H₂O (20 mL) and extracted with DCM (50 mL×3). The combined organic layer was washed with brine (20 mL), dried over Na₂SO₄, filtered and concentrated. The crude product was purified by Pre-HPLC (neutral condition) to give (S)-9,10-difluoro-6-((((2-methylpyridin-4-yl)methyl)(1-(pyrazin-2-yl)piperidin-3-yl)amino)methyl)-2,3-dihydro-7H-[1,4]thiazino[2,3,4-ij]quinolin-7-one (11 mg, 8.84% yield) as a yellow solid. ¹H NMR (DMSO-d6) δ: 8.23-8.37 (m, 2H), 7.96-8.07 (m, 1H), 7.90-7.95 (m, 1H), 7.72-7.85 (m, 2H), 7.16-7.24 (m, 2H), 4.40-4.58 (m, 3H), 4.23-4.32 (m, 1H), 3.55-3.83 (m, 4H), 3.38-3.44 (m, 2H), 2.95-3.05 (m, 1H), 2.74-2.84 (m, 1H), 2.57-2.69, (m, 1H), 2.34-2.38 (m, 3H), 1.60-1.82 (m, 2H), 1.19-1.45 (m, 2H). MS (ESI): mass calcd. For C₂₈H₂₈F₂N₆OS 534.6 m/z found 535.20 [M+H]⁺.

### Compound 98

### Preparation of (S)-6-(((1-(6-((cyclopropylmethyl)amino)pyridin-3-yl)piperidin-3-yl)((2-methoxypyridin-4-yl)methyl)amino)methyl)-9,10-difluoro-2,3-dihydro-7H-[1,4]thiazino[2,3,4-ij]quinolin-7-one (Step 2 in Scheme 20)

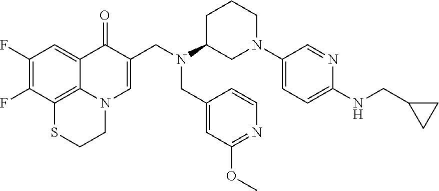 [SMILES: COc1cc(CN(Cc2cn3c4c(c(F)c(F)cc4c2=O)SCC3)[C@H]2CCCN(c3ccc(NCC4CC4)nc3)C2)ccn1]

To a solution of 9,10-difluoro-6-({[(3S)-1-{6-[(cyclopropylmethyl)amino]pyridine-3-yl}hexahydropyridin-3-yl]amino}methyl)-3,7-dihydro-2H-[1,4]thiazino[2,3,4-ij]quinolin-7-one (60 mg, 0.12 mmol) in DMF (1 mL) was added 4-(bromomethyl)-2-methoxypyridine (48.72 mg, 0.24 mmol) and K₂CO₃ (4.17 mg, 0.03 mmol) in portion at room temperature. The reaction mixture was stirred at 80° C. for 3 hours. The reaction mixture was poured into H₂O (30 mL) and extracted with DCM (50 mL×3). The combined organic layer was washed with brine (50 mL), dried over Na₂SO₄, filtered and concentrated. The crude product was purified by Pre-HPLC (neutral) to give(S)-6-(((1-(6-((cyclopropylmethyl)amino)pyridin-3-yl)piperidin-3-yl)((2-methoxypyridin-4-yl)methyl)amino)methyl)-9,10-difluoro-2,3-dihydro-7H-[1,4]thiazino[2,3,4-ij]quinolin-7-one (15 mg, 20.11%) as a yellow solid. ¹H NMR (DMSO-d6) δ: 7.96-7.99 (m, 1H), 7.90-7.94 (m, 1H), 7.73-7.82 (m, 1H), 7.61-7.66 (m, 1H), 7.12-7.19 (m, 1H), 6.95-7.00 (m, 1H), 6.75-6.78 (m, 1H), 6.39-6.44 (m, 1H), 6.01-6.09 (m, 1H), 4.44-4.52 (m, 2H), 3.74-3.77 (m, 3H), 3.68-3.73 (m, 2H), 3.56-3.66 (m, 2H), 3.46-3.56 (m, 2H), 3.39-3.44 (m, 2H), 3.21-3.26 (m, 1H), 3.20-3.26 (m, 1H), 3.01-3.06 (m, 2H), 2.55-2.81 (m, 2H), 1.91-2.03 (m, 2H), 1.41-1.50 (m, 2H), 0.81-0.88 (m, 1H), 0.38-0.43 (m, 2H), 0.14-0.19 (m, 2H). MS (ESI): mass calcd. For C₃₃H₃₆F₂N₆O₂S 618.7 m/z found 619.26 [M+H]⁺.

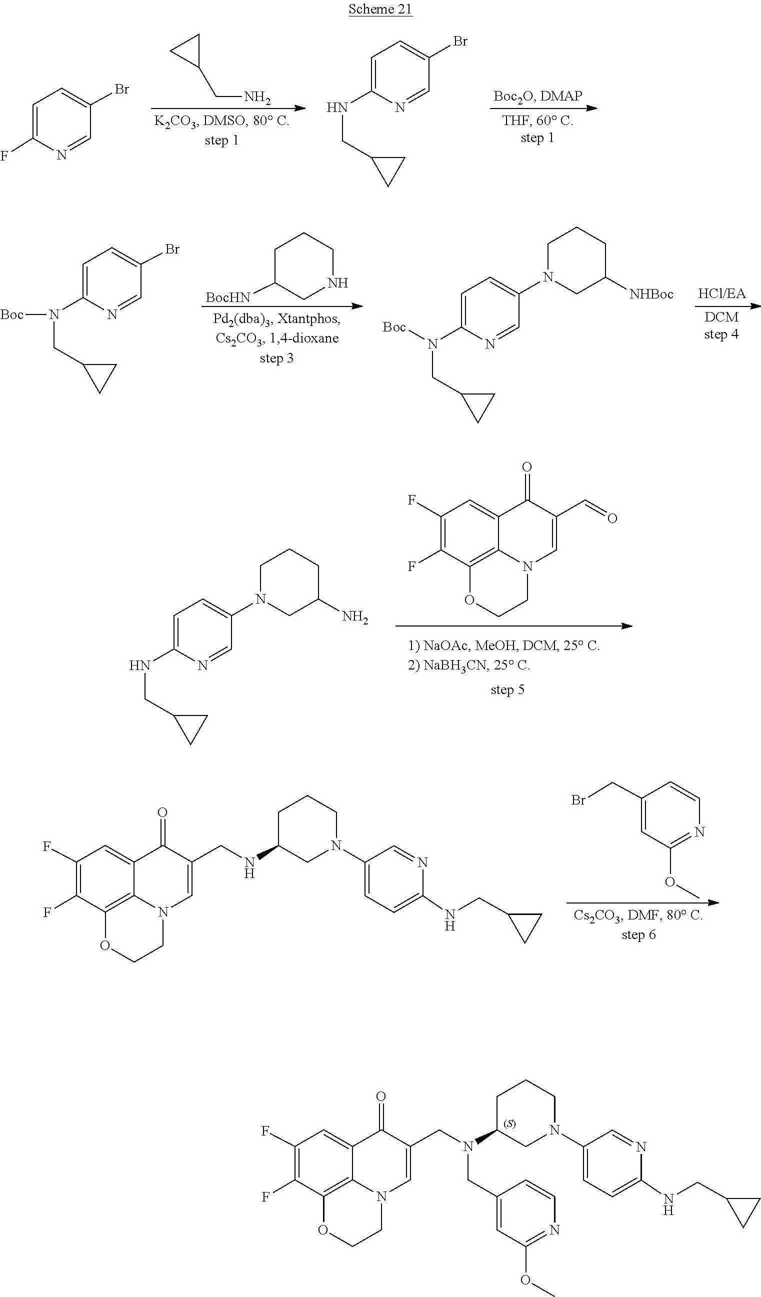

### Preparation of 5-bromo-N-(cyclopropylmethyl)pyridin-2-amine (Step 1 in Scheme 21)

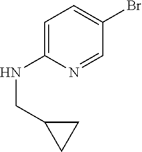 [SMILES: Brc1ccc(NCC2CC2)nc1]

To a mixture of 5-bromo-2-fluoropyridine (8 g, 45.46 mmol) and potassium carbonate (18.85 g, 136.37 mmol) and DMSO (40 mL) was added cyclopropylmethanamine (4.34 mL, 50.00 mmol). The reaction mixture was stirred at 80° C. for overnight. LCMS showed that 5-bromo-2-fluoropyridine was consumed. The reaction mixture was poured into ice-water. Then the reaction mixture was extracted with EA. The combined organic layer was washed with brine, dried over Na₂SO₄, filtered and concentrated. The crude product was purified by column chromatography (SiO₂, PE:EA=20:1) to give 5-bromo-N-(cyclopropylmethyl)pyridin-2-amine (6.15 g, 59.57% yield) as a yellow solid. MS (ESI): mass calcd. For C₉H₁₁BrN₂ 226.01 m/z found 227.0 [M+H]⁺.

### Preparation of tert-butyl (5-bromopyridin-2-yl)(cyclopropylmethyl)carbamate (Step 2 in Scheme 21)

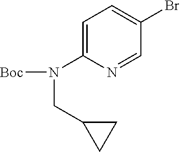 [SMILES: CC(C)(C)OC(=O)N(CC1CC1)c1ccc(Br)cn1]

To a mixture of 5-bromo-N-(cyclopropylmethyl)pyridin-2-amine (6.15 g, 27.08 mmol) and 4-(dimethylamino)pyridine (6.65 g, 54.16 mmol) and dimethyl N,N-tetrahydrofuran (35 mL) was added Boc₂O (18.7 mL, 81.24 mmol). The reaction mixture was stirred at 60° C. for overnight. LCMS showed that 5-bromo-2-[(cyclopropylmethyl)amino]pyridine was consumed. The reaction mixture was poured into ice-water. Then the reaction mixture was extracted with EA. The combined organic layer was washed with brine, dried over Na₂SO₄, filtered and concentrated. The crude product was purified by column chromatography (SiO₂, PE:EA=50:1) to give tert-butyl (5-bromopyridin-2-yl)(cyclopropylmethyl)carbamate (7.588 g, 85.63% yield) as a yellow oil. MS (ESI): mass calcd. For Cl₄H₁₉BrN₂O₂ 326.06 m/z found 271.0 [M+H−56]⁺.

### Preparation of tert-butyl (5-(3-((tert-butoxycarbonyl)amino)piperidin-1-yl)pyridin-2-yl)(cyclopropylmethyl)carbamate (Step 3 in Scheme 21)

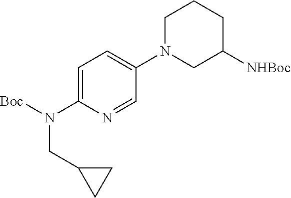 [SMILES: CC(C)(C)OC(=O)NC1CCCN(c2ccc(N(CC3CC3)C(=O)OC(C)(C)C)nc2)C1]

To a mixture of tert-butyl (5-bromopyridin-2-yl)(cyclopropylmethyl)carbamate (7.58 g, 23.16 mmol), Xtantphos (0.80 g, 1.39 mmol), Cs₂CO₃ (10.19 g, 31.27 mmol) and Pd₂(dba)₃ (1.27 g, 1.39 mmol) in dioxane (40 mL) was added 2-methylpropan-2-yl (hexahydropyridin-3-ylamino)methanoate (6.03 g, 30.11 mmol). The reaction mixture was stirred at 90° C. for overnight. LCMS showed that 5-bromo-2-[(cyclopropylmethyl)amino]pyridine was consumed. The reaction mixture was poured into ice-water. Then the reaction mixture was extracted with EA. The combined organic layer was washed with brine, dried over Na₂SO₄, filtered and concentrated. The crude product was purified by column chromatography (SiO₂, DCM:EA=6:1) to give tert-butyl (5-(3-((tert-butoxycarbonyl)amino)piperidin-1-yl)pyridin-2-yl)(cyclopropylmethyl)carbamate (833 mg, 8.05% yield) as a yellow solid. MS (ESI): mass calcd. For C₂₄H₃₈N₄O₄ 446.29 m/z found 447.3 [M+H]⁺.

### Preparation of 5-(3-aminopiperidin-1-yl)-N-(cyclopropylmethyl)pyridin-2-amine (Step 4 in Scheme 21)

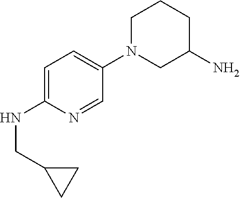 [SMILES: NC1CCCN(c2ccc(NCC3CC3)nc2)C1]

To a solution of tert-butyl (5-(3-((tert-butoxycarbonyl)amino)piperidin-1-yl)pyridin-2-yl)(cyclopropylmethyl)carbamate (833 mg, 1.87 mmol) in DCM (1 mL) was added HCl/EA (5 mL). The reaction mixture was stirred at 25° C. for overnight. LCMS showed that tert-butyl (5-(3-((tert-butoxycarbonyl)amino)piperidin-1-yl)pyridin-2-yl)(cyclopropylmethyl)carbamate was consumed. The reaction mixture was adjusted to pH=7 with NH₃·H₂O. The crude product was purified by column chromatography (SiO₂, DCM:MeOH=10:1) to give 5-(3-aminopiperidin-1-yl)-N-(cyclopropylmethyl)pyridin-2-amine (393 mg, 85.52% yield) as a yellow oil. MS (ESI): mass calcd. For C₁₄H₂₂N₄ 246.18 m/z found 247.2 [M+H]⁺.

### Preparation of (S)-6-(((1-(6-((cyclopropylmethyl)amino)pyridin-3-yl)piperidin-3-yl)amino)methyl)-9,10-difluoro-2,3-dihydro-7H-[1,4]oxazino[2,3,4-ij]quinolin-7-one (Step 5 in Scheme 21)

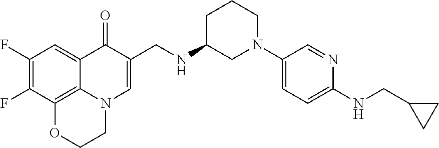 [SMILES: O=c1c(CN[C@H]2CCCN(c3ccc(NCC4CC4)nc3)C2)cn2c3c(c(F)c(F)cc13)OCC2]

To a mixture of 9,10-difluoro-7-oxo-2,3-dihydro-7H-[1,4]oxazino[2,3,4-ij]quinoline-6-carbaldehyde (100 mg, 0.40 mmol) and sodium acetate (33 mg, 0.40 mmol) in DCM (1 mL) and MeOH (1 mL) was added 5-(3-aminopiperidin-1-yl)-N-(cyclopropylmethyl)pyridin-2-amine (98 mg, 0.40 mmol). The reaction mixture was stirred at 25° C. for 1 hour. NaBH₃CN (100 mg, 1.59 mmol) was added to the reaction mixture. The reaction mixture was stirred at 25° C. for overnight. LCMS showed that 9,10-difluoro-7-oxo-2,3-dihydro-7H-[1,4]oxazino[2,3,4-ij]quinoline-6-carbaldehyde was consumed. The reaction mixture was poured into ice-water. Then the reaction mixture was extracted with EA. The combined organic layer was washed with brine, dried over Na₂SO₄, filtered and concentrated. The crude product was purified by column chromatography (DCM:MeOH=10:1) to give (S)-6-(((1-(6-((cyclopropylmethyl)amino)pyridin-3-yl)piperidin-3-yl)amino)methyl)-9,10-difluoro-2,3-dihydro-7H-[1,4]oxazino[2,3,4-ij]quinolin-7-one (107 mg, 55.81% yield) as a yellow solid. MS (ESI): mass calcd. For C₂₆H₂₉F₂N₅O₂ 481.23 m/z found 482.2 [M+H]⁺.

### Compound 99

### Preparation of (S)-6-(((1-(6-((cyclopropylmethyl)amino)pyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (Step 6 in Scheme 21)

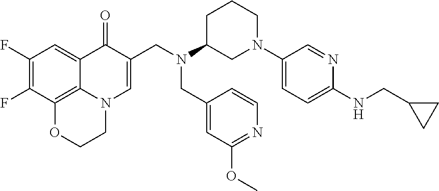 [SMILES: COc1cc(CN(Cc2cn3c4c(c(F)c(F)cc4c2=O)OCC3)[C@H]2CCCN(c3ccc(NCC4CC4)nc3)C2)ccn1]

To a mixture of (S)-6-(((1-(6-((cyclopropylmethyl)amino)pyridin-3-yl)piperidin-3-yl)amino)methyl)-9,10-difluoro-2,3-dihydro-7H-[1,4]oxazino[2,3,4-ij]quinolin-7-one (107 mg, 0.22 mmol) and Cs₂CO₃ (145 mg, 0.44 mmol) in N,N-dimethylmethanamide (1.5 mL) was added 4-(bromomethyl)-2-methoxypyridine (90 mg, 0.44 mmol). The reaction mixture was stirred at 80° C. for 2 hours. LCMS showed that (S)-6-(((1-(6-((cyclopropylmethyl)amino)pyridin-3-yl)piperidin-3-yl)amino)methyl)-9,10-difluoro-2,3-dihydro-7H-[1,4]oxazino[2,3,4-ij]quinolin-7-one was consumed. The reaction mixture was poured into ice-water. Then the reaction mixture was extracted with EA. The combined organic layer was washed with brine, dried over Na₂SO₄, filtered and concentrated. The crude product was purified by column chromatography (SiO₂, DCM:MeOH=10:1-5:1) to give (S)-6-(((1-(6-((cyclopropylmethyl)amino)pyridin-3-yl)piperidin-3-yl)((2-methoxypyridin-4-yl)methyl)amino)methyl)-9,10-difluoro-2,3-dihydro-7H-[1,4]oxazino[2,3,4-ij]quinolin-7-one (10 mg, 7.47% yield) as a yellow solid. ¹H NMR (DMSO-d₆, 400 MHz) δ: 7.95-8.02 (m, 2H), 7.62-7.65 (m, 1H), 7.49-7.56 (m, 1H), 7.16 (br d, J=8.3 Hz, 1H), 6.97-7.02 (m, 1H), 6.79 (s, 1H), 6.40-6.45 (m, 1H), 6.04-6.13 (m, 1H), 4.58 (br s, 2H), 4.35 (br s, 2H), 3.76 (s, 3H), 3.71 (br d, J=6.6 Hz, 2H), 3.59 (br d, J=17.3 Hz, 2H), 3.47-3.52 (m, 2H), 3.03 (br s, 2H), 1.92-2.00 (m, 1H), 1.74 (br d, J=10.9 Hz, 1H), 1.23 (br s, 2H), 0.95-1.05 (m, 1H), 0.40 (br d, J=7.8 Hz, 2H), 0.13-0.19 (m, 2H). MS (ESI): mass calcd. For C₃₃H₃₆F₂N₆O₃ 602.28 m/z found 603.2 [M+H]⁺.

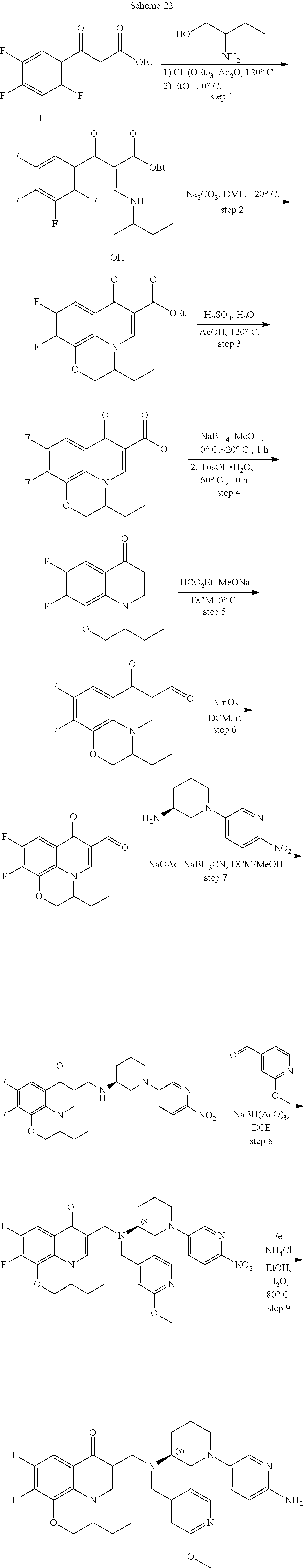

### Preparation of ethyl (Z)-3-((1-hydroxybutan-2-yl)amino)-2-(2,3,4,5-tetrafluorobenzoyl)acrylate (Step 1 in Scheme 22)

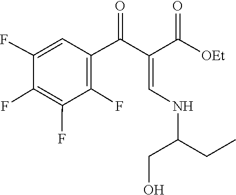 [SMILES: CCOC(=O)/C(=C\NC(CC)CO)C(=O)c1cc(F)c(F)c(F)c1F]

To a solution of ethyl 3-oxo-3-(2,3,4,5-tetrafluorophenyl)propanoate (7.25 mL, 37.85 mmol) in acetic anhydride (35.45 mL, 378.53 mmol) was added triethoxy methane (1.26 mL, 7.57 mmol) in portion wise at room temperature. The reaction mixture was stirred at 120° C. for 2 hours. The reaction mixture was concentrated and diluted with EtOH (20 mL). 2-aminobutan-1-ol (3.55 mL, 37.85 mmol) was added to the reaction mixture in portion at 0° C. The reaction mixture was stirred at 0° C. for 1 hour. The reaction mixture was poured into H₂O (200 mL) and extracted with EA (200 mL×3). The combined organic layer was washed with brine (200 mL), dried over Na₂SO₄, filtered and concentrated. The crude product was purified by column chromatography (SiO₂, PE:EA=2:1) to give ethyl (Z)-3-((1-hydroxybutan-2-yl)amino)-2-(2,3,4,5-tetrafluorobenzoyl)acrylate (13.48 g, 98.00% yield) as a yellow oil.

### Preparation of ethyl 3-ethyl-9,10-difluoro-7-oxo-2,3-dihydro-7H-[1,4]oxazino[2,3,4-ij]quinoline-6-carboxylate (Step 2 in Scheme 22)

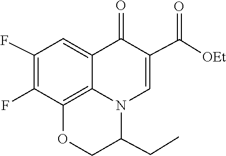 [SMILES: CCOC(=O)c1cn2c3c(c(F)c(F)cc3c1=O)OCC2CC]

To a solution of ethyl (2Z)-3-[(1-hydroxybut-2-yl)amino]-2-[(2,3,4,5-tetrafluorophenyl)carbonyl]prop-2-enoate (5.0 g, 13.76 mmol) in N,N-dimethylmethanamide (40 mL) was added sodium carbonate (4.38 g, 41.28 mmol) in portion at room temperature. The reaction mixture was stirred at 120° C. for overnight. The reaction mixture was poured into H₂O (150 mL) and extracted with EA (80 mL×3). The combined organic layer was washed with brine (100 mL), dried over Na₂SO₄, filtered and concentrated to give crude product ethyl 3-ethyl-9,10-difluoro-7-oxo-2,3-dihydro-7H-[1,4]oxazino[2,3,4-ij]quinoline-6-carboxylate (3.3 g, 74.17% yield) as a yellow solid. MS (ESI): mass calcd. For C₁₆H₁₅F₂NO₄ 323.3 m/z found 324.10 [M+H]⁺.

### Preparation of 3-ethyl-9,10-difluoro-7-oxo-2,3-dihydro-7H-[1,4]oxazino[2,3,4-ij]quinoline-6-carboxylic Acid (Step 3 in Scheme 22)

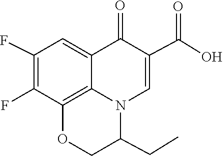 [SMILES: CCC1COc2c(F)c(F)cc3c(=O)c(C(=O)O)cn1c23]

To a solution of ethyl 9,10-difluoro-7-oxo-3-(prop-2-yl)-2,3-dihydro[1,4]oxazino[2,3,4-ij]quinoline-6-carboxylate (3.3 g, 9.78 mmol) in AcOH (15 mL) and H₂O (3 mL) was added H₂SO₄ (2 mL, 0.30 mmol) in portion at room temperature. The reaction mixture was stirred at 110° C. for 2 hours. The reaction mixture was cooled to room temperature and poured into H₂O (100 mL). The mixture was filtered to give crude product 3-ethyl-9,10-difluoro-7-oxo-2,3-dihydro-7H-[1,4]oxazino[2,3,4-ij]quinoline-6-carboxylic acid (3.0 g, 99.55% yield) as a yellow solid.

### Preparation of 3-ethyl-9,10-difluoro-2.3,5,6-tetrahydro-7H-[1,4]oxazino[2,3,4-ij]quinolin-7-one (Step 4 in Scheme 22)

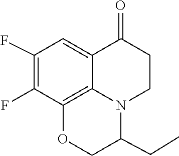 [SMILES: CCC1COc2c(F)c(F)cc3c2N1CCC3=O]

To a solution of 3-ethyl-9,10-difluoro-7-oxo-2,3-dihydro[1,4]oxazino[2,3,4-ij]quinoline-6-carboxylic acid (3.0 g, 10.16 mmol) in MeOH (30 mL) was added NaBH₄ (1.69 g, 44.71 mmol) in portion at 0° C. The reaction mixture was stirred at room temperature for 1 hour. TsOH (0.24 g, 1.42 mmol) was added to the reaction mixture in portion. The reaction mixture was stirred at 60° C. for overnight. The reaction mixture was adjusted to pH=5 with 2M HCl and extracted with EA (50 mL×3). The combined organic layer was washed with brine (50 mL), dried over Na₂SO₄, filtered and concentrated. The crude product was purified by column chromatography (SiO₂, PE:EA=4:1) to give 3-ethyl-9,10-difluoro-2,3,5,6-tetrahydro-7H-[1,4]oxazino[2,3,4-ij]quinolin-7-one (700 mg, 27.20% yield) as a yellow solid. MS (ESI): mass calcd. For C₁₃H₁₃F₂NO₂ 253.2 m/z found 254.09[M+H]⁺.

### Preparation of 3-ethyl-9,10-difluoro-7-oxo-2,3,6,7-tetrahydro-5H-[1,4]oxazino[2,3,4-ij]quinoline-6-carbaldehyde (Step 5 in Scheme 22)

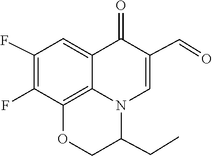 [SMILES: CCC1COc2c(F)c(F)cc3c(=O)c(C=O)cn1c23]

To a solution of 3-ethyl-9,10-difluoro-3,5,6,7-tetrahydro-2H-[1,4]oxazino[2,3,4-ij]quinolin-7-one (700 mg, 2.76 mmol) in DCM (5 mL) was added CH₃ONa (597.26 mg, 11.06 mmol) and HCO₂Et (0.86 mL, 10.50 mmol) in portion at 0° C. The reaction mixture was stirred at room temperature for 30 minutes. The reaction mixture was adjusted to pH=5 with 2M HCl and extracted with EA (200 mL×3). The combined organic layer was washed with brine (100 mL), dried over Na₂SO₄, filtered and concentrated to give crude 3-ethyl-9,10-difluoro-7-oxo-2,3,6,7-tetrahydro-5H-[1,4]oxazino[2,3,4-ij]quinoline-6-carbaldehyde (600 mg, 77.18% yield) as a yellow solid.

### Preparation of 3-ethyl-9,10-difluoro-2,3,5,6-tetrahydro-7H-[1,4]oxazino[2,3,4-ij]quinolin-7-one (Step 6 in Scheme 22)

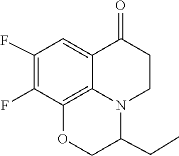 [SMILES: CCC1COc2c(F)c(F)cc3c2N1CCC3=O]

To a solution of 3-ethyl-9,10-difluoro-7-oxo-3,5,6,7-tetrahydro-2H-[1,4]thiazino[2,3,4-ij]quinoline-6-carbaldehyde (660 mg, 2.22 mmol) in DCM (5 mL) was added manganese dioxide (2315.90 mg, 26.64 mmol) in portion at room temperature. The reaction mixture was stirred at room temperature for 2 hours. The reaction mixture was filtered and washed with MeOH/DCM (5:1). The filtrate was concentrated to give crude 3-ethyl-9,10-difluoro-2,3,5,6-tetrahydro-7H-[1,4]oxazino[2,3,4-ij]quinolin-7-one (640 mg, 97.63% yield) as a gray solid. MS (ESI): mass calcd. For C₁₄H₁₁F₂NO₃ 279.2 m/z found 280.07[M+H]⁺.

### Preparation of 3-ethyl-9,10-difluoro-6-((((S)-1-(6-nitropyridin-3-yl)piperidin-3-yl)amino)methyl)-2,3-dihydro-7H-[1,4]oxazino[2,3,4-ij]quinolin-7-one (Step 7 in Scheme 22)

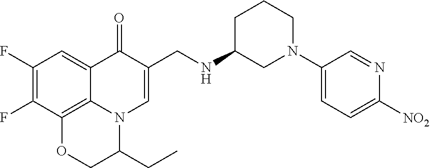 [SMILES: CCC1COc2c(F)c(F)cc3c(=O)c(CN[C@H]4CCCN(c5ccc([N+](=O)[O-])nc5)C4)cn1c23]

To a solution of 3-ethyl-9,10-difluoro-7-oxo-2,3-dihydro[1,4]oxazino[2,3,4-ij]quinoline-6-carbaldehyde (640 mg, 2.29 mmol) in MeOH (3 mL) and DCM (6 mL) was added 1-(6-nitropyridin-3-yl)hexahydropyridin-3-amine (509.38 mg, 2.29 mmol) and NaOAc (188.01 mg, 2.29 mmol) in portion at room temperature. The reaction mixture was stirred at room temperature for 2 hours. NaBH₃CN (260.11 mg, 6.88 mmol) was added to the reaction mixture in portion. The reaction mixture was stirred at room temperature. for 1 hour. The reaction mixture was poured into H₂O (30 mL) and extracted with DCM (50 mL×3). The combined organic layer was washed with brine (50 mL), dried over Na₂SO₄, filtered and concentrated. The crude product was purified by column chromatography (SiO₂, DCM:MeOH=5:1) to give 3-ethyl-9,10-difluoro-6-((((S)-1-(6-nitropyridin-3-yl)piperidin-3-yl)amino)methyl)-2,3-dihydro-7H-[1,4]oxazino[2,3,4-ij]quinolin-7-one (1.0 g, 89.87% yield) as a yellow solid. MS (ESI): mass calcd. For C₂₄H₂₅F₂N₅O₄ 485.2 m/z found 486.19 [M+H]⁺.

### Preparation of 3-ethyl-9,10-difluoro-6-((((2-methoxypyridin-4-yl)methyl)((S)-1-(6-nitropyridin-3-yl)piperidin-3-yl)amino)methyl)-2,3-dihydro-7H-[1,4]oxazino[2,3,4-ij]quinolin-7-one (Step 8 in Scheme 22)

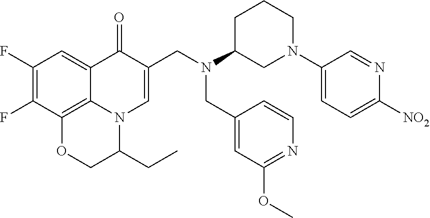 [SMILES: CCC1COc2c(F)c(F)cc3c(=O)c(CN(Cc4ccnc(OC)c4)[C@H]4CCCN(c5ccc([N+](=O)[O-])nc5)C4)cn1c23]

To a solution of 3-ethyl-9,10-difluoro-6-({[(3S)-1-(6-nitropyridin-3-yl)hexahydropyridin-3-yl]amino}methyl)-3,7-dihydro-2H-[1,4]oxazino[2,3,4-ij]quinolin-7-one (300 mg, 0.62 mmol) in DCE (2 mL) was added 2-methoxypyridine-4-carbaldehyde (254.23 mg, 1.85 mmol) in portion at room temperature. The reaction mixture was stirred at room temperature for 30 minutes. NaBH(AcO)₃ (392.89 mg, 1.85 mmol) and 2-methoxypyridine-4-carbaldehyde (254.23 mg, 1.85 mmol) was added to the reaction mixture in portion. The reaction mixture was stirred at room temperature for 1 hour. The reaction mixture was poured into H₂O (100 mL) and extracted with DCM (150 mL×3). The combined organic layer was washed with brine (100 mL), dried over Na₂SO₄, filtered and concentrated. The crude product was purified by Pre-HPLC (neutral condition) to give 3-ethyl-9,10-difluoro-6-((((2-methoxypyridin-4-yl)methyl)((S)-1-(6-nitropyridin-3-yl)piperidin-3-yl)amino)methyl)-2,3-dihydro-7H-[1,4]oxazino[2,3,4-ij]quinolin-7-one (360 mg, 96.04% yield) as a yellow solid. MS (ESI): mass calcd. For C₃₁H₃₂F₂N₆O₅ 606.6 m/z found 607.24 [M+H]⁺.

### Compound 100

### Preparation of 6-((((S)-1-(6-aminopyridin-3-yl)piperidin-3-yl)((2-methoxypyridin-4-yl)methyl)amino)methyl)-3-ethyl-9,10-difluoro-2,3-dihydro-7H-[1,4]oxazino[2,3,4-ij]quinolin-7-one (Step 9 in Scheme 22)

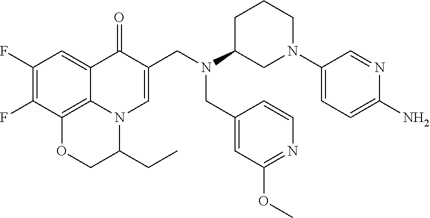 [SMILES: CCC1COc2c(F)c(F)cc3c(=O)c(CN(Cc4ccnc(OC)c4)[C@H]4CCCN(c5ccc(N)nc5)C4)cn1c23]

To a solution of 9,10-difluoro-6-({[(2-methoxypyridin-4-yl)methyl][(3S)-1-(6-nitropyridin-3-yl)hexahydropyridin-3-yl]amino}methyl)-3-methyl-3,7-dihydro-2H-[1,4]oxazino[2,3,4-ij]quinolin-7-one (430 mg, 0.73 mmol) in EtOH (5 mL) and H₂O (2 mL) was added Fe (0.04 mL, 5.93 mmol) and NH₄Cl (0.21 mL, 5.93 mmol) in portion at room temperature. The reaction mixture was stirred at 80° C. for 2 hours. The reaction mixture was poured into EA (50 mL) and filtered. The filtrate was extracted with EA (100 mL×3). The combined organic layer was washed with brine (50 mL), dried over Na₂SO₄, filtered and concentrated. The crude product was purified by Pre-HPLC (neutral condition) to give 6-((((S)-1-(6-aminopyridin-3-yl)piperidin-3-yl)((2-methoxypyridin-4-yl)methyl)amino)methyl)-3-ethyl-9,10-difluoro-2,3-dihydro-7H-[1,4]oxazino[2,3,4-ij]quinolin-7-one (288 mg, 84.16% yield) as a yellow solid. ¹H NMR (DMSO-d6) δ: 8.11-8.17 (m, 1H), 8.07 (d, J=5.1 Hz, 1H), 7.89-7.97 (m, 1H), 7.74 (br s, 2H), 7.58 (br t, J=9.1 Hz, 1H), 7.41 (br s, 1H), 7.07 (br d, J=5.1 Hz, 1H), 6.89-7.01 (m, 2H), 4.62-4.88 (m, 3H), 4.43 (br d, J=6.4 Hz, 2H), 4.31-4.39 (m, 2H), 4.14-4.27 (m, 2H), 3.96-4.12 (m, 2H), 3.75-3.79 (m, 3H), 3.35-3.44 (m, 1H), 3.05-3.28 (m, 1H), 2.91 (br d, J=11.8 Hz, 1H), 2.53-2.64 (m, 1H), 2.11-2.24 (m, 1H), 1.81-1.95 (m, 1H), 1.69-1.81 (m, 2H), 1.55-1.69 (m, 2H), 0.89 (q, J=7.1 Hz, 3H). MS (ESI): mass calcd. For C₃₁H₃₄F₂N₆O₃ 576.6 m/z found 577.27 [M+H]⁺.

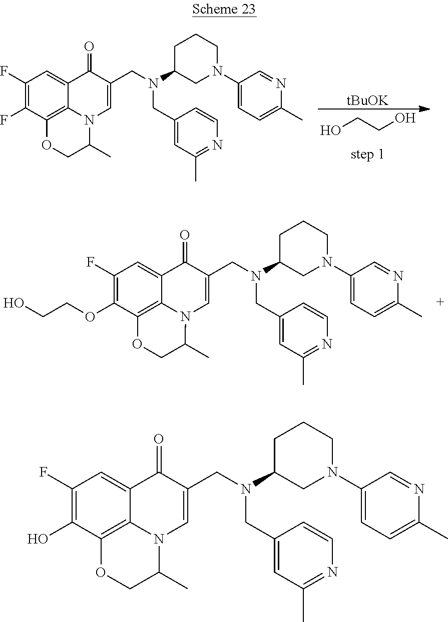

### Compound 101

### Preparation of 9-fluoro-10-(2-hydroxyethoxy)-3-methyl-6-((((S)-1-(6-methylpyridin-3-yl)piperidin-3-yl)((2-methylpyridin-4-yl)methyl)amino)methyl)-2,3-dihydro-7H-[1,4]oxazino[2,3,4-ij]quinolin-7-one (Step 1 in Scheme 23)

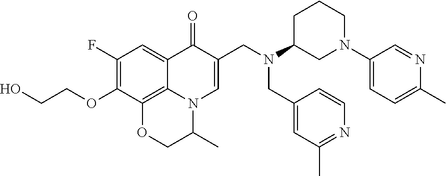 [SMILES: Cc1ccc(N2CCC[C@H](N(Cc3ccnc(C)c3)Cc3cn4c5c(c(OCCO)c(F)cc5c3=O)OCC4C)C2)cn1]

### Compound 102

### Preparation of 9-fluoro-10-hydroxy-3-methyl-6-((((S)-1-(6-methylpyridin-3-yl)piperidin-3-yl)((2-methylpyridin-4-yl)methyl)amino)methyl)-2,3-dihydro-7H-[1,4]oxazino[2,3,4-ij]quinolin-7-one (Step 1 in Scheme 23)

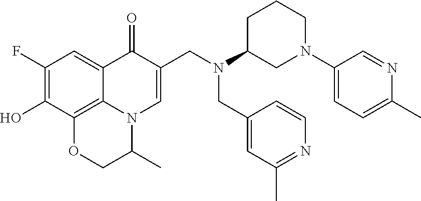 [SMILES: Cc1ccc(N2CCC[C@H](N(Cc3ccnc(C)c3)Cc3cn4c5c(c(O)c(F)cc5c3=O)OCC4C)C2)cn1]

A mixture of 9,10-difluoro-3-methyl-6-((((S)-1-(6-methylpyridin-3-yl)piperidin-3-yl)((2-methylpyridin-4-yl)methyl)amino)methyl)-2,3-dihydro-7H-[1,4]oxazino [2,3,4-ij]quinolin-7-one (380 mg, 0.697 mmol), tBuOK (234 mg, 2.09 mmol) and glycol (5 mL) was stirred at 120° C. for 16 hours under N₂ atmosphere. TLC showed the reaction was complete. The mixture was filtered. The filtrate was concentrated to give residue. The residue was purified by prep-HPLC with H₂O(NH₄HCO₃)/ACN to afford 9-fluoro-10-(2-hydroxyethoxy)-3-methyl-6-((((S)-1-(6-methylpyridin-3-yl)piperidin-3-yl)((2-methylpyridin-4-yl)methyl)amino)methyl)-2,3-dihydro-7H-[1,4]oxazino[2,3,4-ij]quinolin-7-one (52 mg) and 9-fluoro-10-hydroxy-3-methyl-6-((((S)-1-(6-methylpyridin-3-yl)piperidin-3-yl)((2-methylpyridin-4-yl)methyl)amino)methyl)-2,3-dihydro-7H-[1,4]oxazino[2,3,4-ij]quinolin-7-one (26 mg). ¹H NMR (CHLOROFORM-d, 400 MHz) δ 8.3-8.4 (m, 1H), 8.1-8.2 (m, 1H), 7.7-7.8 (m, 1H), 7.5-7.6 (m, 1H), 7.1-7.1 (m, 3H), 7.0-7.0 (m, 1H), 4.3-4.4 (m, 1H), 4.3-4.3 (m, 3H), 4.2-4.2 (m, 1H), 3.8-3.9 (m, 3H), 3.7-3.8 (m, 4H), 3.5-3.6 (m, 1H), 2.9-3.0 (m, 1H), 2.7-2.8 (m, 1H), 2.6-2.7 (m, 1H), 2.45 (dd, 6H, J=1.7, 3.4 Hz), 2.1-2.1 (m, 1H), 1.9-1.9 (m, 1H), 1.5-1.7 (m, 2H), 1.4-1.5 (m, 3H). MS (ESI): mass calcd. For C₃₃H₃₈FN₅O₄ 587.2 m/z found 588.29 [M+1]⁺. ¹H NMR (CHLOROFORM-d, 400 MHz) δ 8.3-8.4 (m, 1H), 8.1-8.2 (m, 1H), 7.6-7.7 (m, 1H), 7.5-7.5 (m, 1H), 7.1-7.2 (m, 3H), 7.0-7.0 (m, 1H), 4.2-4.3 (m, 2H), 4.1-4.2 (m, 1H), 3.7-3.8 (m, 5H), 3.52 (br d, 1H, J=11.9 Hz), 2.9-3.0 (m, 1H), 2.7-2.8 (m, 1H), 2.6-2.6 (m, 1H), 2.46 (d, 6H, J=4.1 Hz), 2.12 (br d, 1H, J=11.5 Hz), 1.8-1.9 (m, 1H), 1.5-1.7 (m, 2H), 1.3-1.4 (m, 3H). MS (ESI): mass calcd. For C₃₁H₃₄FN₅O₃ 543.2 m/z found 544.26 [M+1]⁺

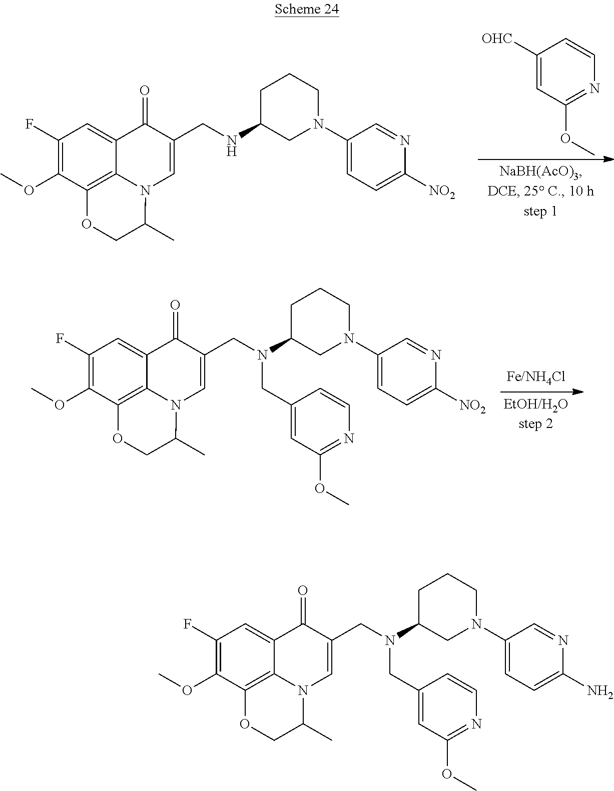

### Preparation of 9-fluoro-10-methoxy-6-((((2-methoxypyridin-4-yl)methyl)((S)-1-(6-nitropyridin-3-yl)piperidin-3-yl)amino)methyl)-3-methyl-2,3-dihydro-7H-[1,4]oxazino[2,3,4-ij]quinolin-7-one (Step 1 in Scheme 24)

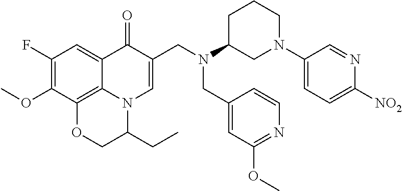 [SMILES: CCC1COc2c(OC)c(F)cc3c(=O)c(CN(Cc4ccnc(OC)c4)[C@H]4CCCN(c5ccc([N+](=O)[O-])nc5)C4)cn1c23]

A mixture of 9-fluoro-10-methoxy-3-methyl-6-((((S)-1-(6-nitropyridin-3-yl)piperidin-3-yl)amino)methyl)-2,3-dihydro-7H-[1,4]oxazino[2,3,4-ij]quinolin-7-one (1 g, 2.07 mmol) and 2-methoxyisonicotinaldehyde (425 mg, 3.11 mmol) in DCE (10 mL) was stirred at 25° C. under N₂ atmosphere for 1 hour. NaBH(OAc)₃ (878 mg, 4.14 mmol) was added to the mixture at 0° C. under N₂ atmosphere. The resulting mixture was stirred at 25° C. for 2 hours. TLC showed the reaction was complete. The mixture was poured into ice-water, extracted with EA and washed with brine. The organic layer was dried over by Na₂SO₄ and concentrated under vacuum. The residue was purified by flash column (eluted with EA/PE) to afford 9-fluoro-10-methoxy-6-((((2-methoxypyridin-4-yl)methyl)((S)-1-(6-nitropyridin-3-yl)piperidin-3-yl)amino)methyl)-3-methyl-2,3-dihydro-7H-[1,4]oxazino[2,3,4-ij]quinolin-7-one (900 mg) as a yellow solid. MS(ESI): mass calcd. For C₃₁H₃₃FN₆O₆ 604.2 m/z found 605.24 [M+1]⁺.

### Compound 103

### Preparation of 6-((((S)-1-(6-aminopyridin-3-yl)piperidin-3-yl)((2-methoxypyridin-4-yl)methyl)amino)methyl)-9-fluoro-10-methoxy-3-methyl-2,3-dihydro-7H-[1,4]oxazino[2,3,4-ij]quinolin-7-one (Step 2 in Scheme 24)

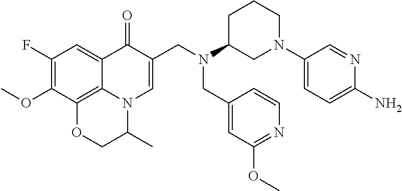 [SMILES: COc1cc(CN(Cc2cn3c4c(c(OC)c(F)cc4c2=O)OCC3C)[C@H]2CCCN(c3ccc(N)nc3)C2)ccn1]

A mixture of 9-fluoro-10-methoxy-6-((((2-methoxypyridin-4-yl)methyl) ((S)-1-(6-nitropyridin-3-yl)piperidin-3-yl)amino)methyl)-3-methyl-2,3-dihydro-7H-[1,4]oxazino[2,3,4-ij]quinolin-7-one (400 mg, 0.66 mmol), Fe (185 mg, 3.31 mmol) and NH₄Cl (351 mg, 6.62 mmol) in EtOH (6 mL)/H₂O (3 mL) was stirred at 80° C. for 3 hours under N₂ atmosphere. TLC showed the reaction was complete. The mixture was filtered. The filtrate was concentrated. The residue was purified by prep-HPLC with H₂O(NH₄HCO₃)/ACN to afford 6-((((S)-1-(6-aminopyridin-3-yl)piperidin-3-yl)((2-methoxypyridin-4-yl)methyl)amino)methyl)-9-fluoro-10-methoxy-3-methyl-2,3-dihydro-7H-[1,4]oxazino[2,3,4-ij]quinolin-7-one (220 mg) as a yellow solid. ¹H NMR (CHLOROFORM-d, 400 MHz) δ 8.0-8.1 (m, 1H), 7.7-7.8 (m, 1H), 7.7-7.7 (m, 1H), 7.6-7.7 (m, 1H), 7.1-7.2 (m, 1H), 6.8-6.9 (m, 1H), 6.7-6.8 (m, 1H), 6.4-6.5 (m, 1H), 4.4-4.4 (m, 1H), 4.3-4.3 (m, 1H), 4.2-4.2 (m, 1H), 4.0-4.1 (m, 3H), 3.9-3.9 (m, 3H), 3.7-3.9 (m, 4H), 3.53 (br d, 1H, J=10.8 Hz), 3.3-3.3 (m, 1H), 3.0-3.0 (m, 1H), 2.9-3.0 (m, 1H), 2.7-2.7 (m, 1H), 2.6-2.7 (m, 1H), 2.5-2.6 (m, 1H), 2.0-2.1 (m, 1H), 1.85 (br s, 1H), 1.6-1.7 (m, 1H), 1.5-1.6 (m, 1H), 1.4-1.5 (m, 3H). MS(ESI): mass calcd. For C₃₁H₃₅FN₆O₄ 574.2 m/z found 575.27 [M+1]⁺.

The compounds in Table S1 can be prepared according to Scheme HB-1 through Scheme HB-14.

TABLE S1

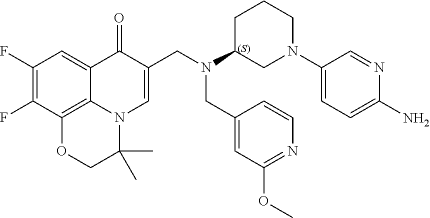

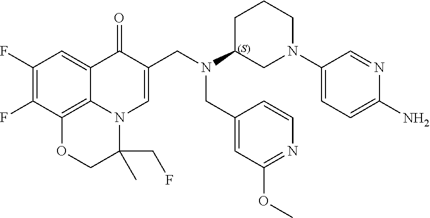

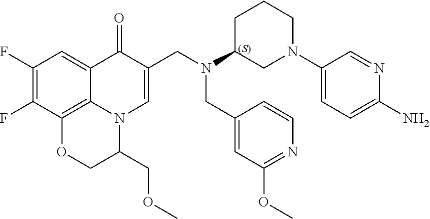

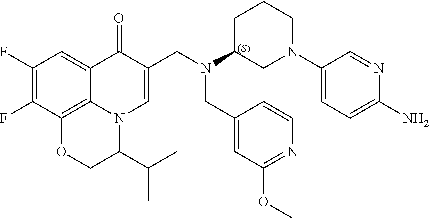

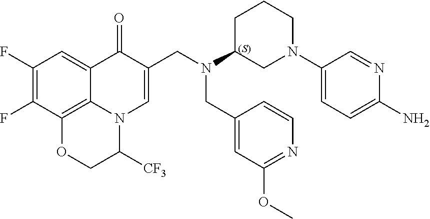

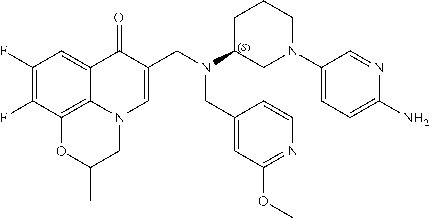

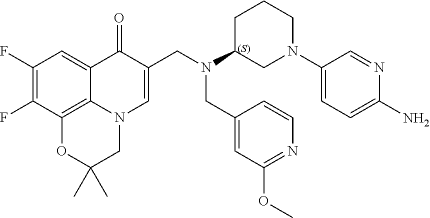

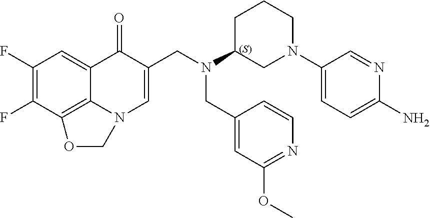

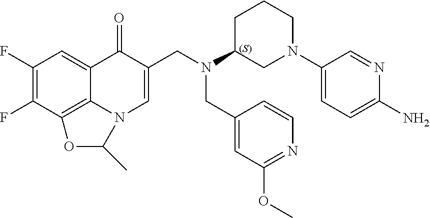

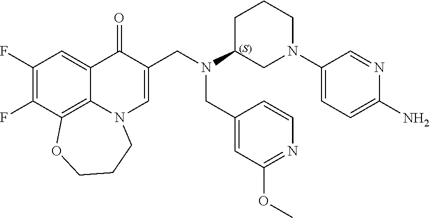

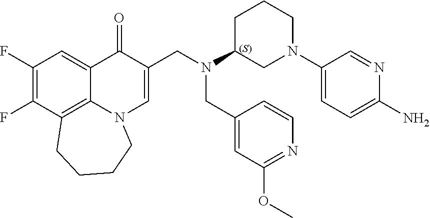

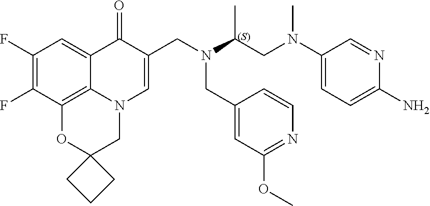

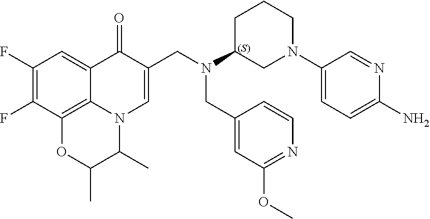

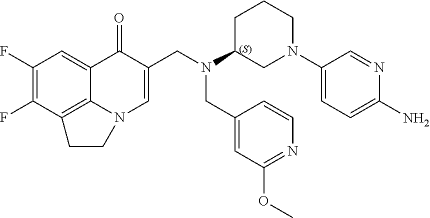

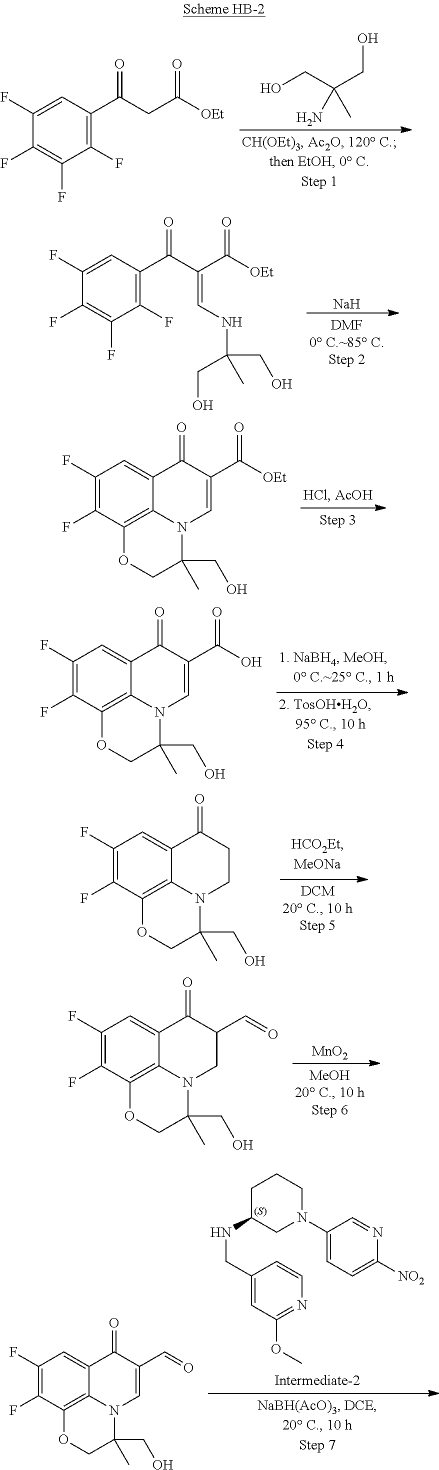

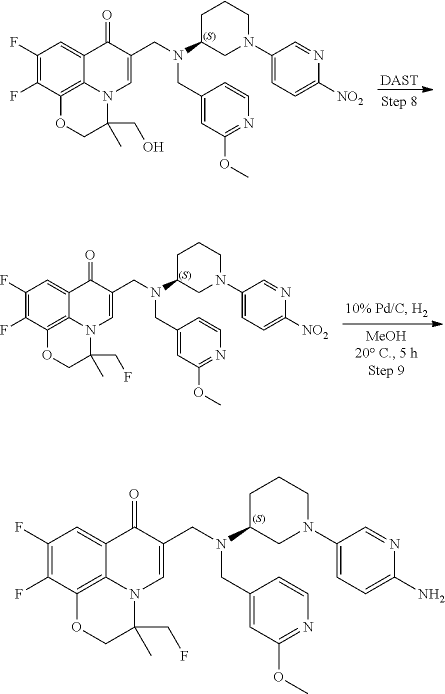

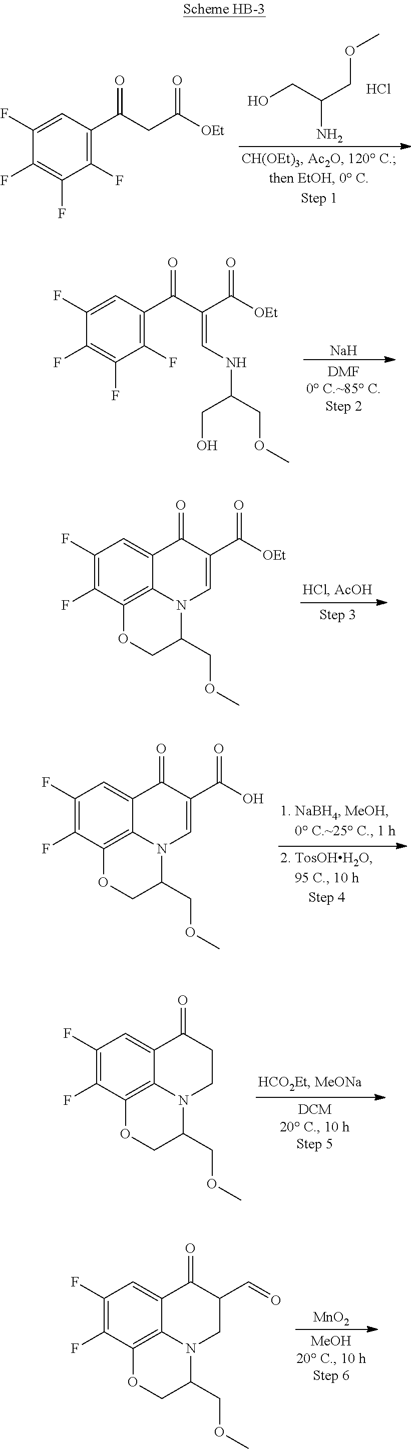

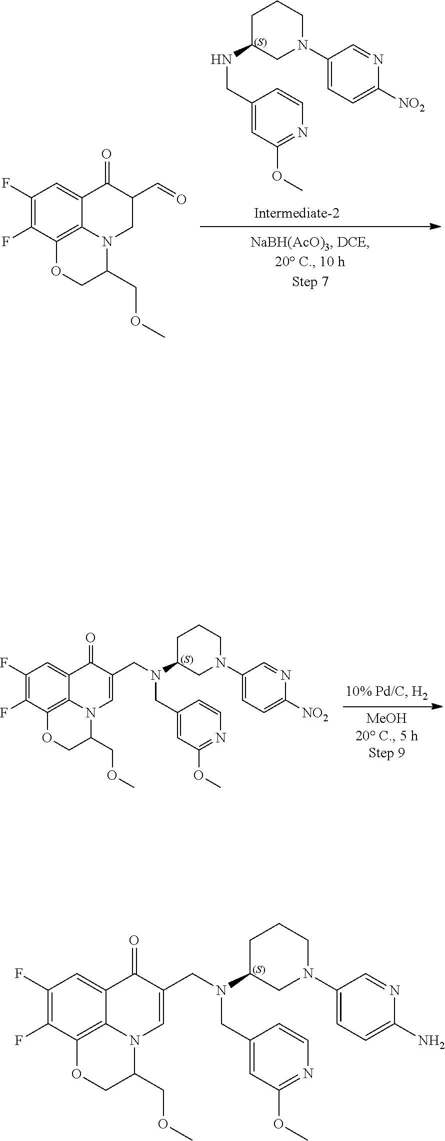

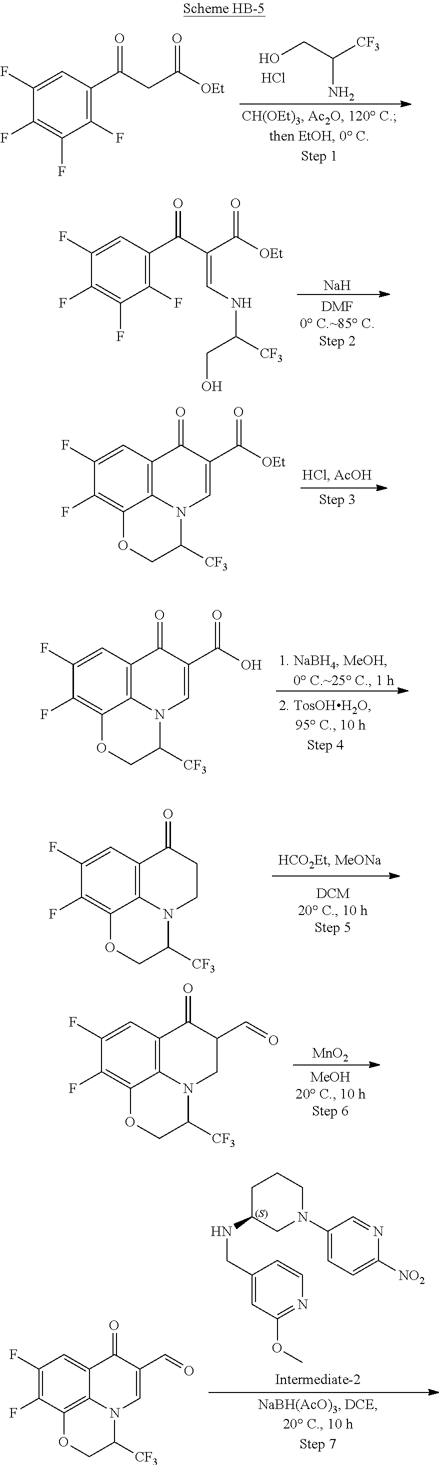

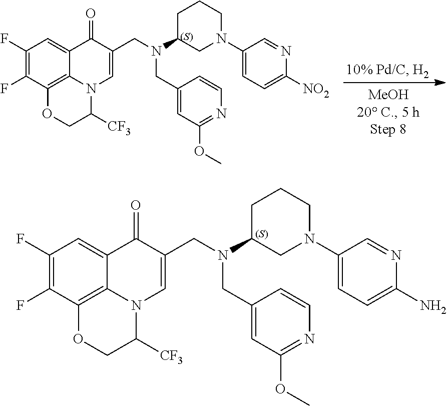

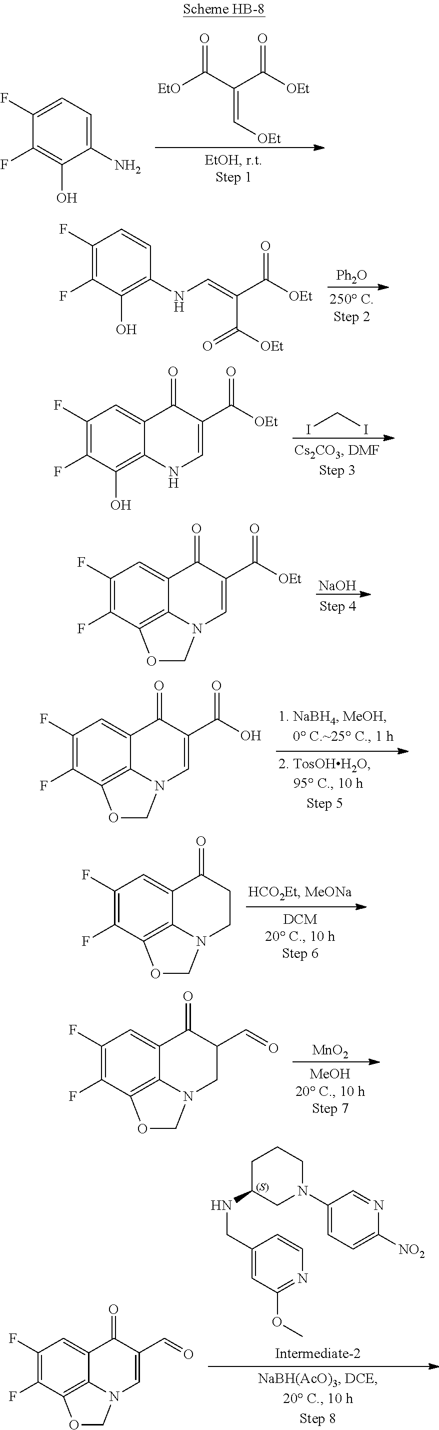

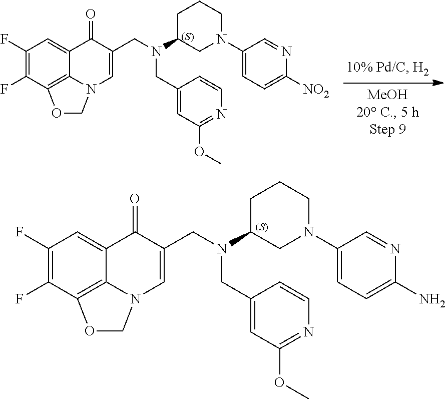

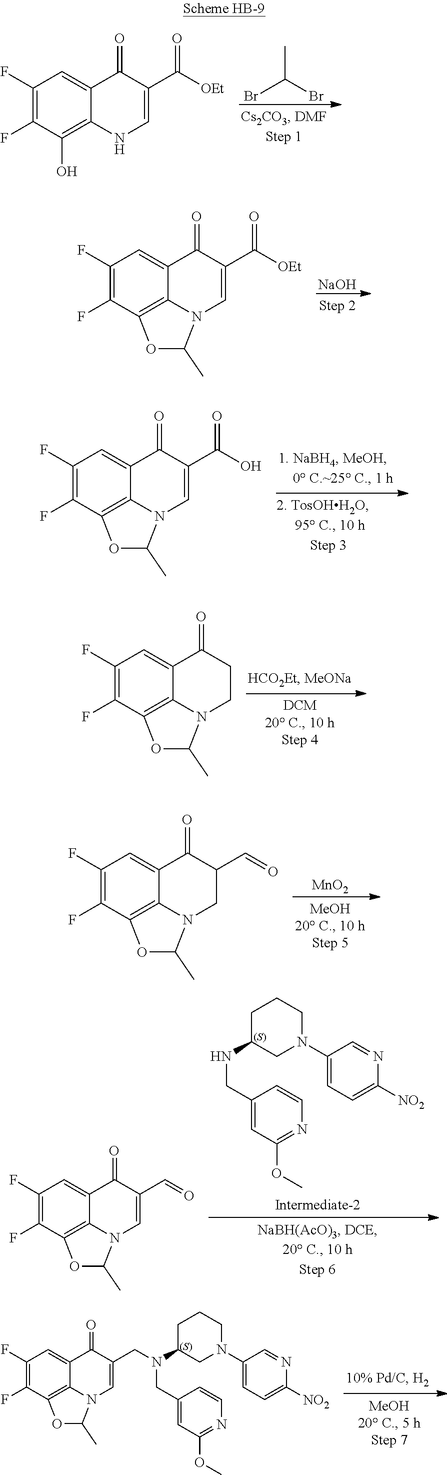

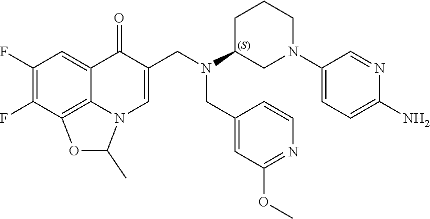

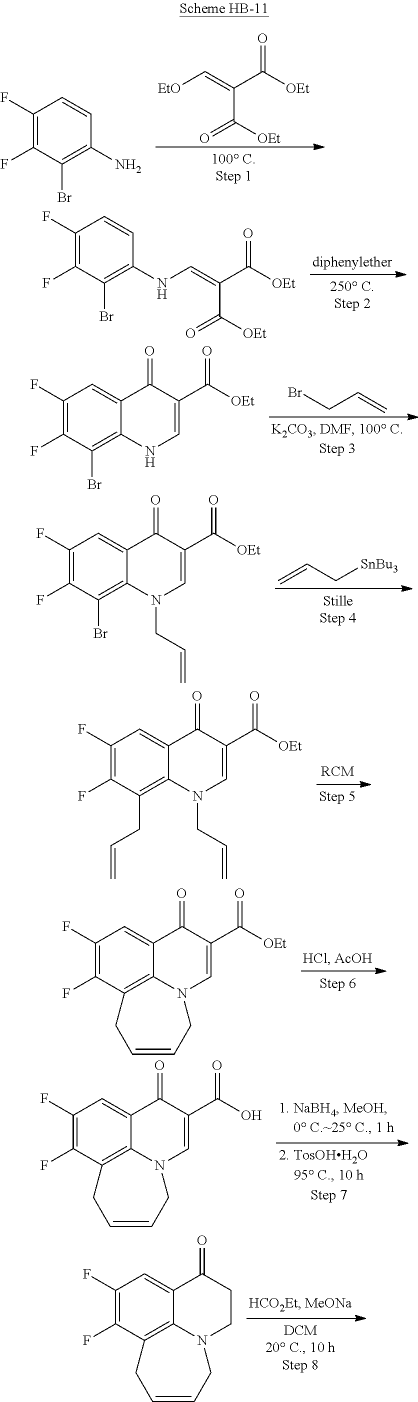

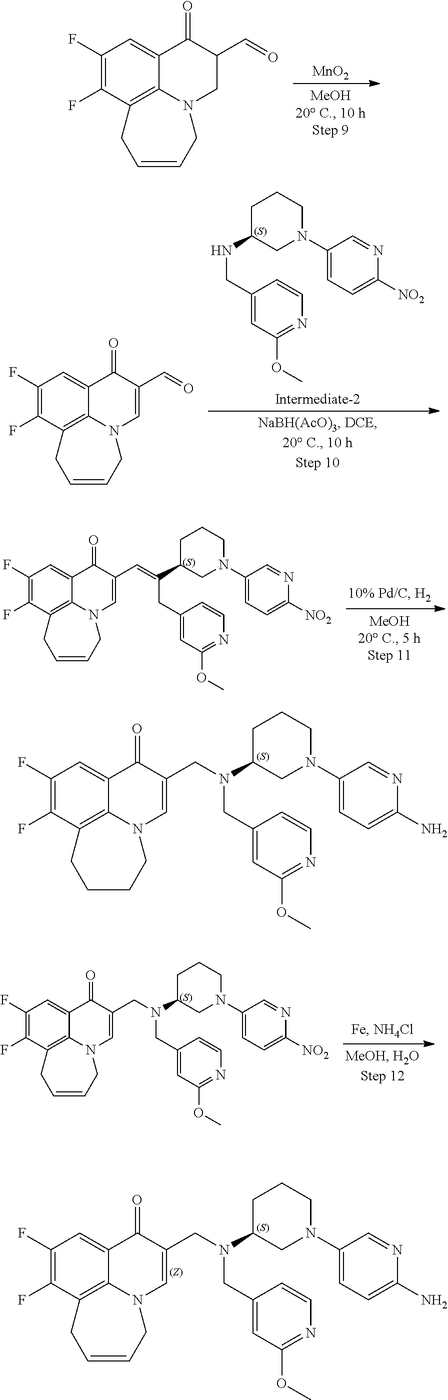

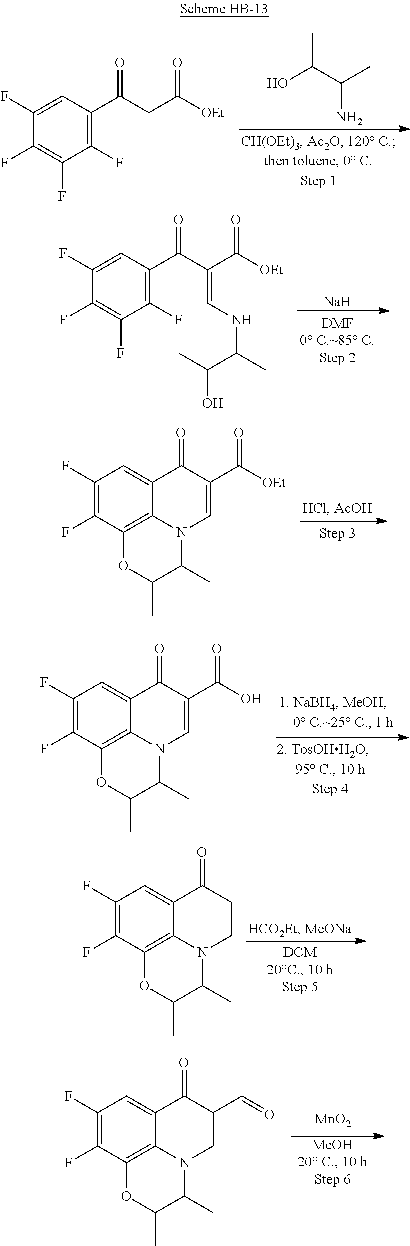

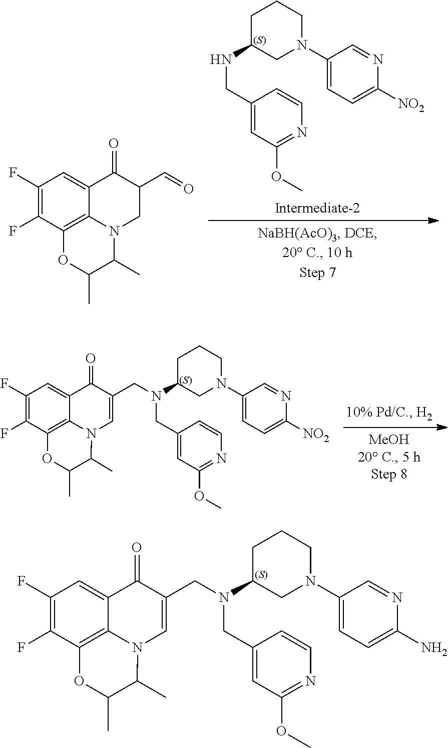

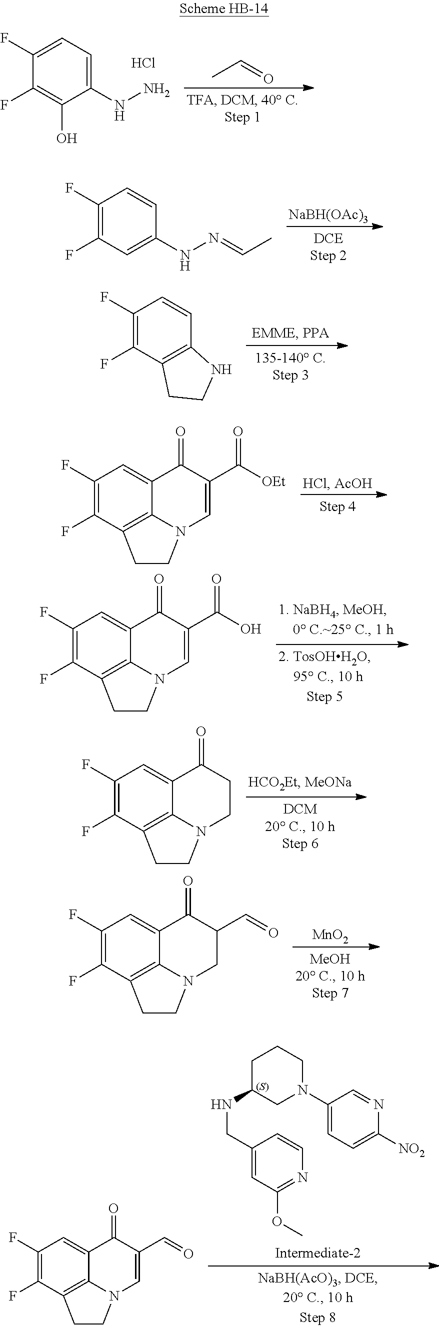

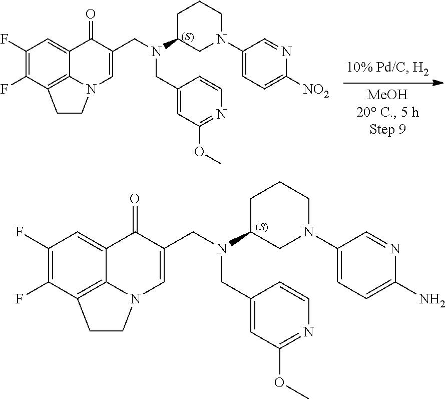

The compounds in Table S2 can be prepared according to Scheme HC-1 through Scheme HC-7.

TABLE S2

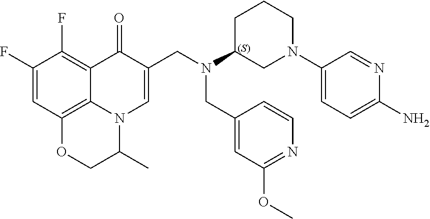

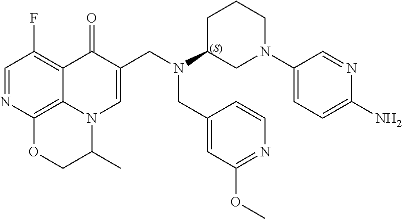

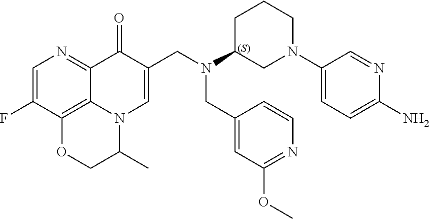

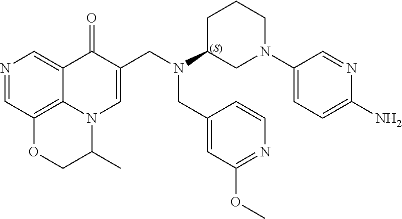

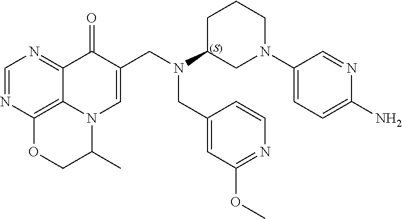

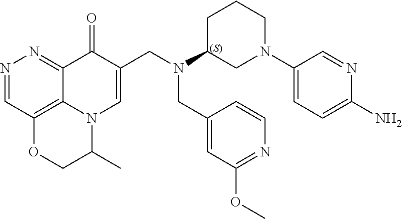

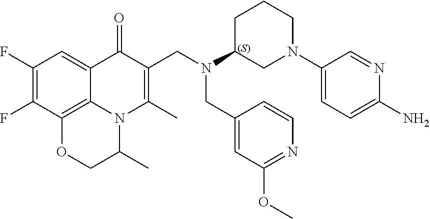

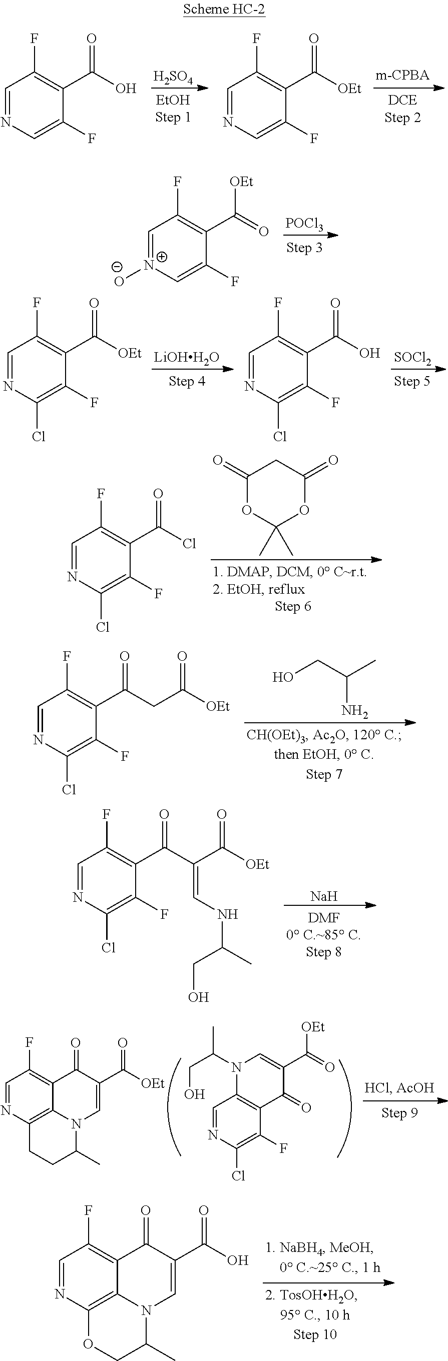

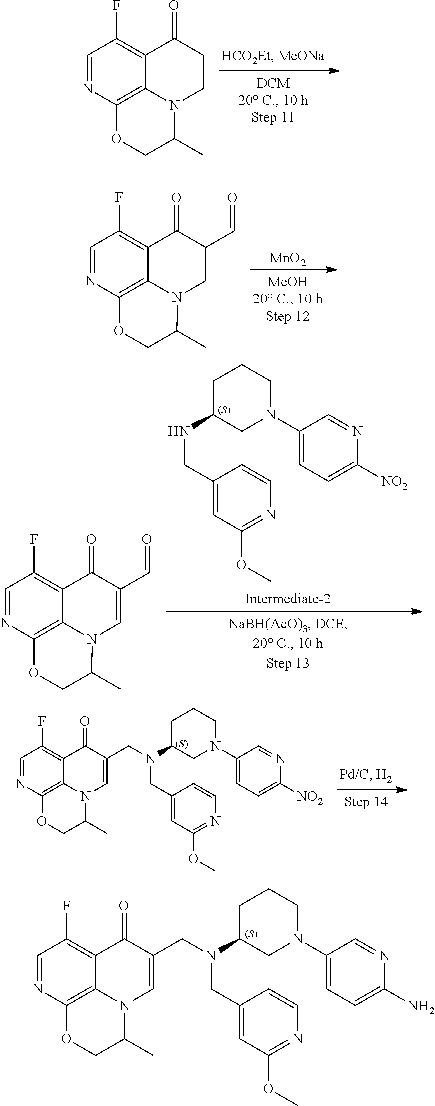

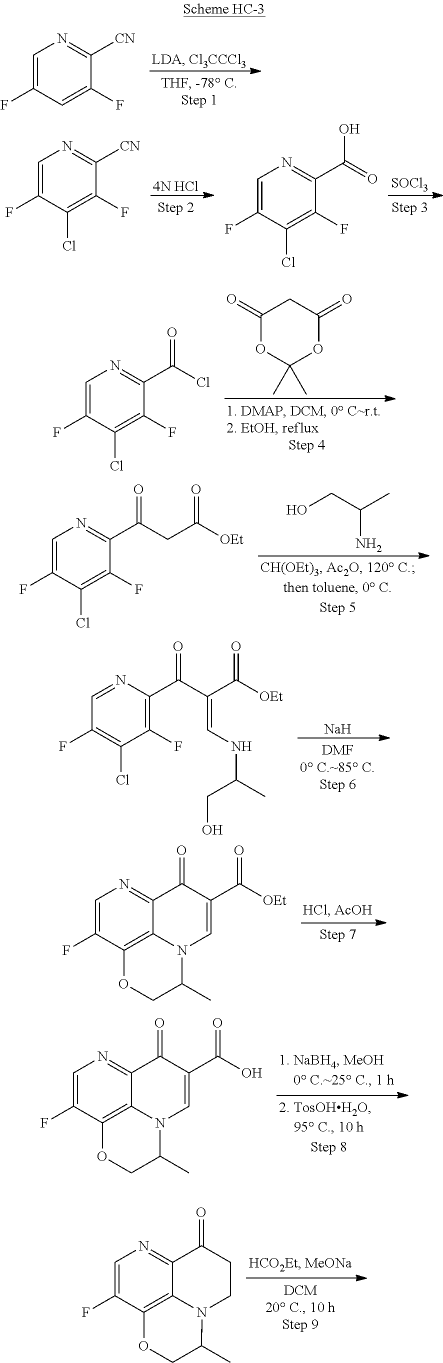

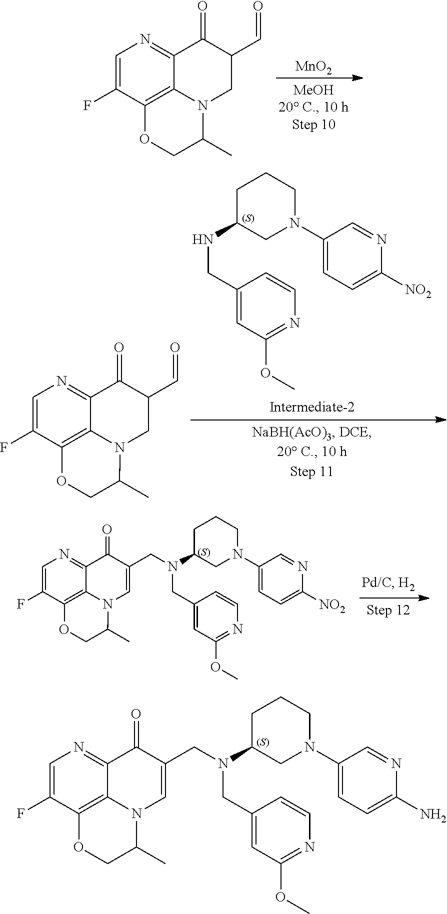

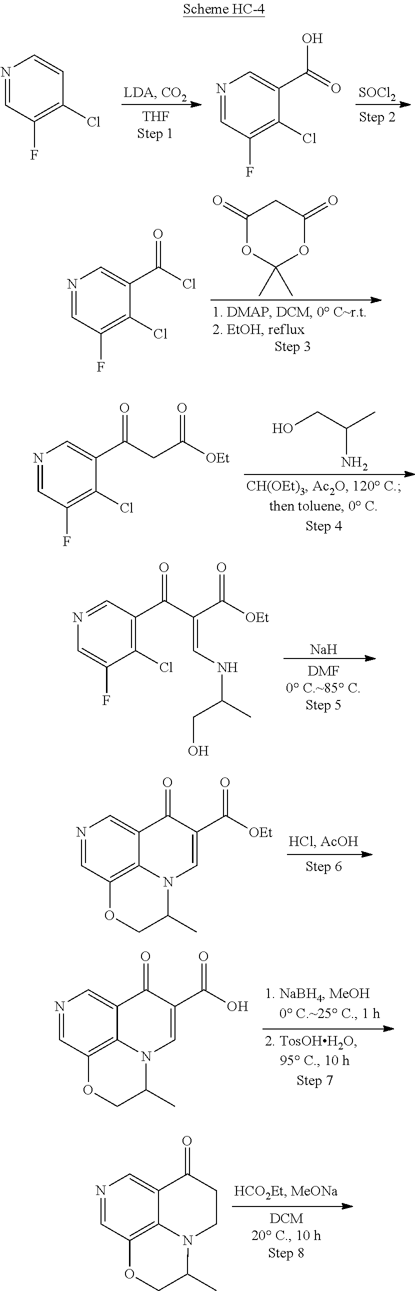

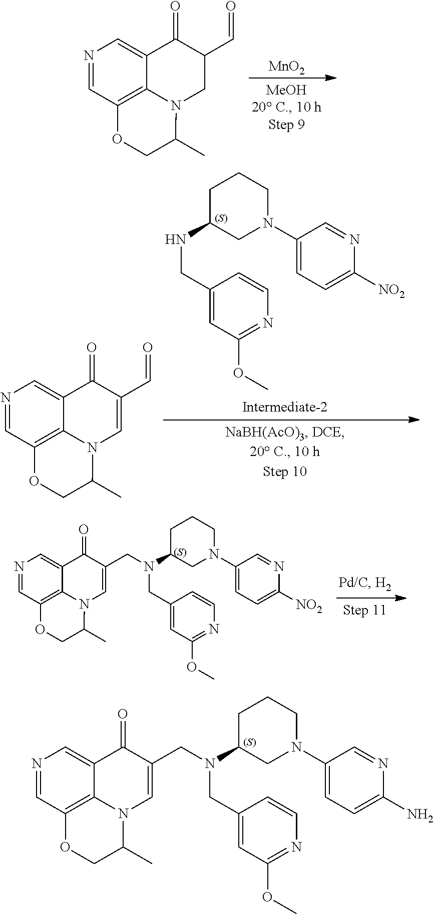

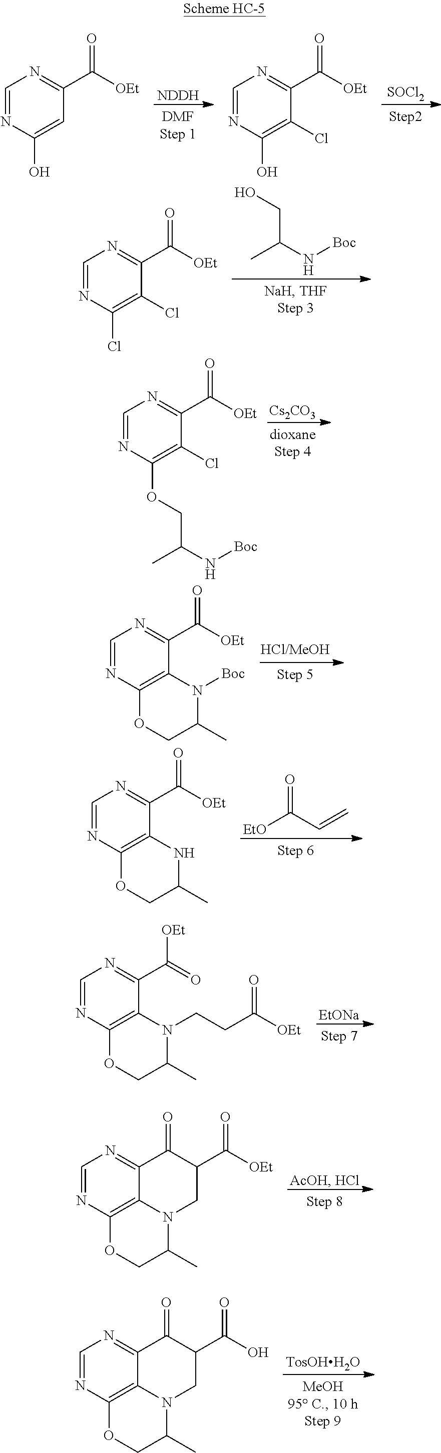

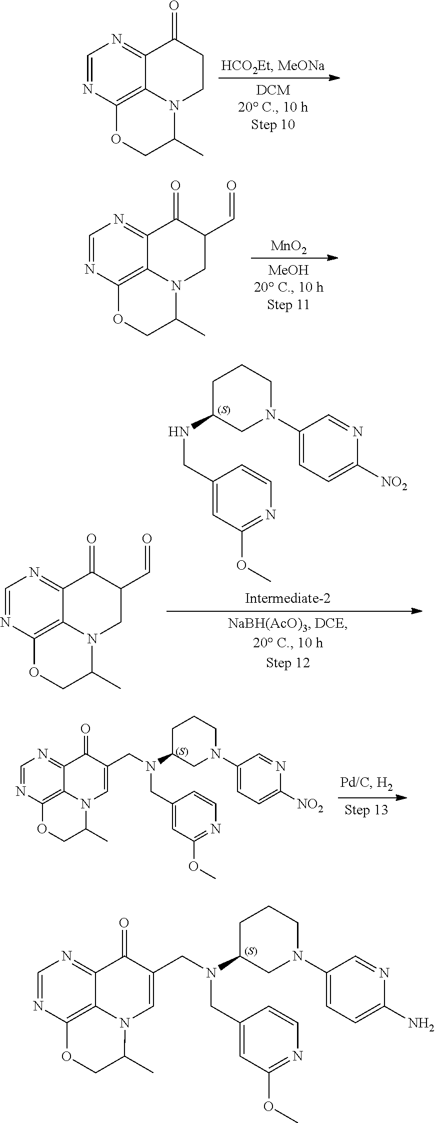

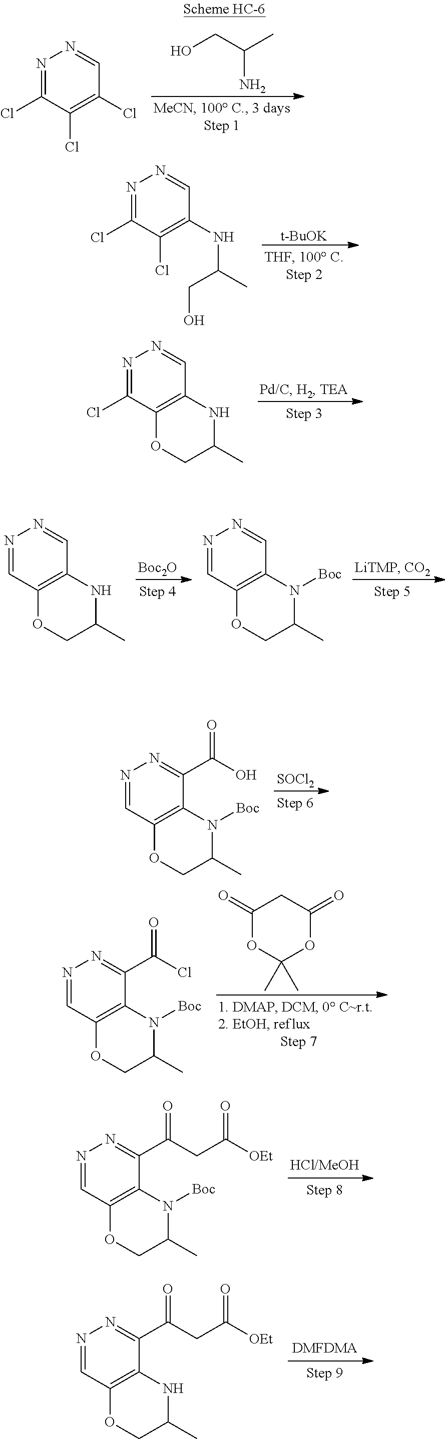

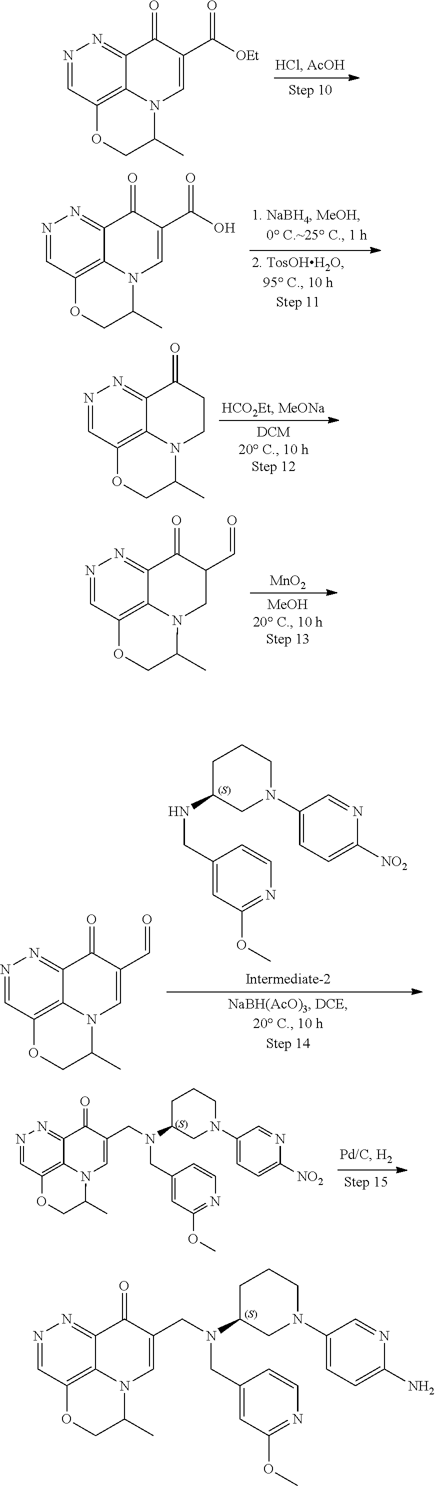

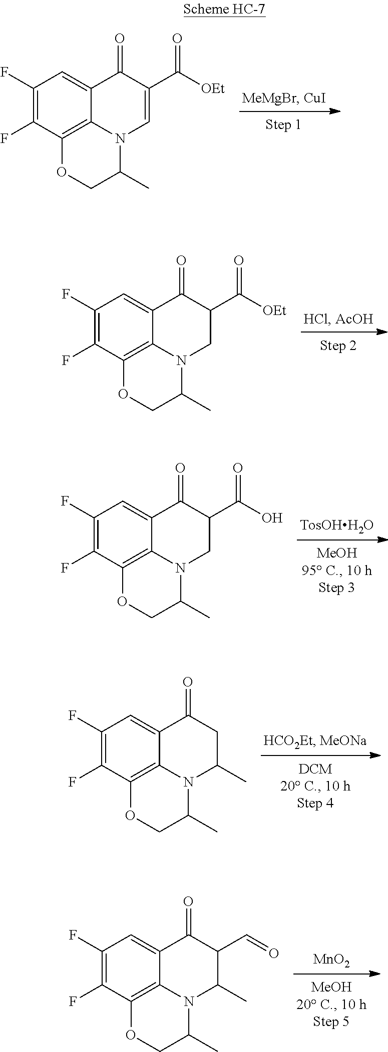

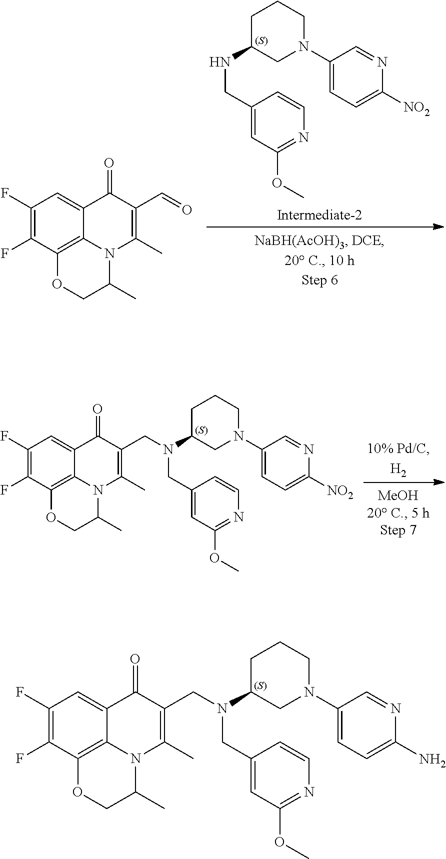

The compounds in Table S3 can be prepared according to Scheme HD-1 through Scheme HD-6.

TABLE S3

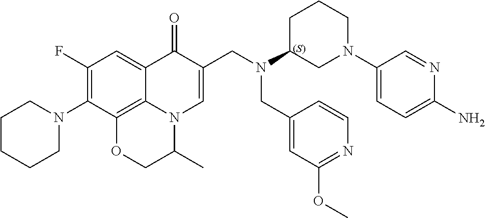

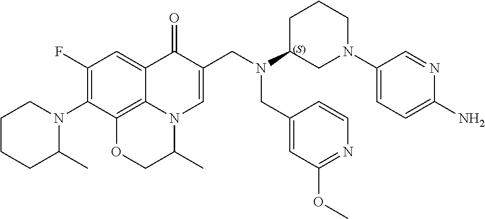

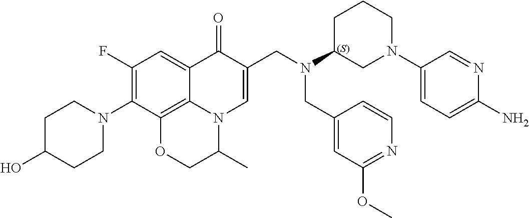

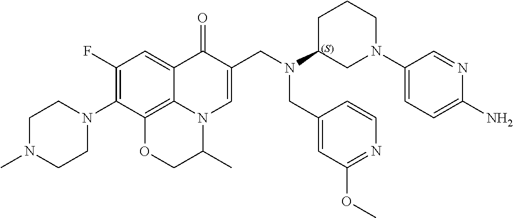

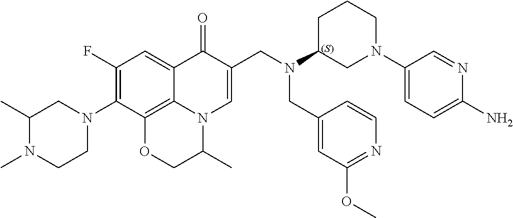

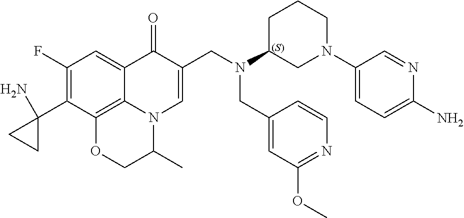

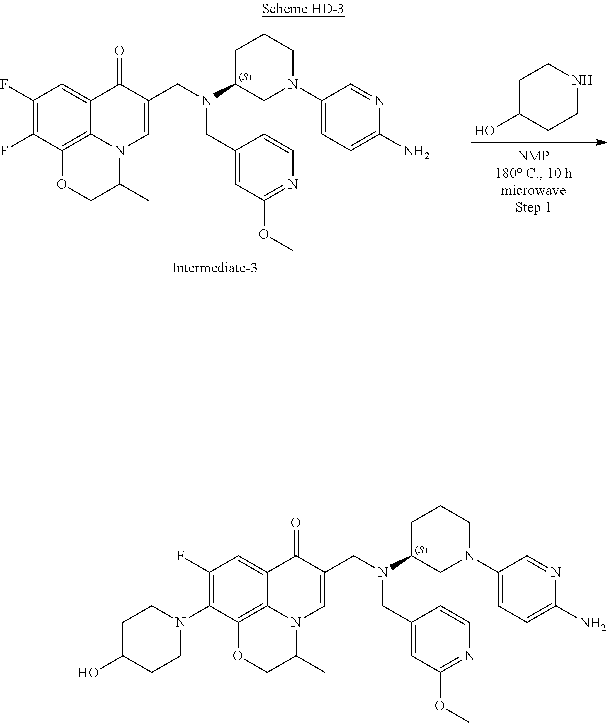

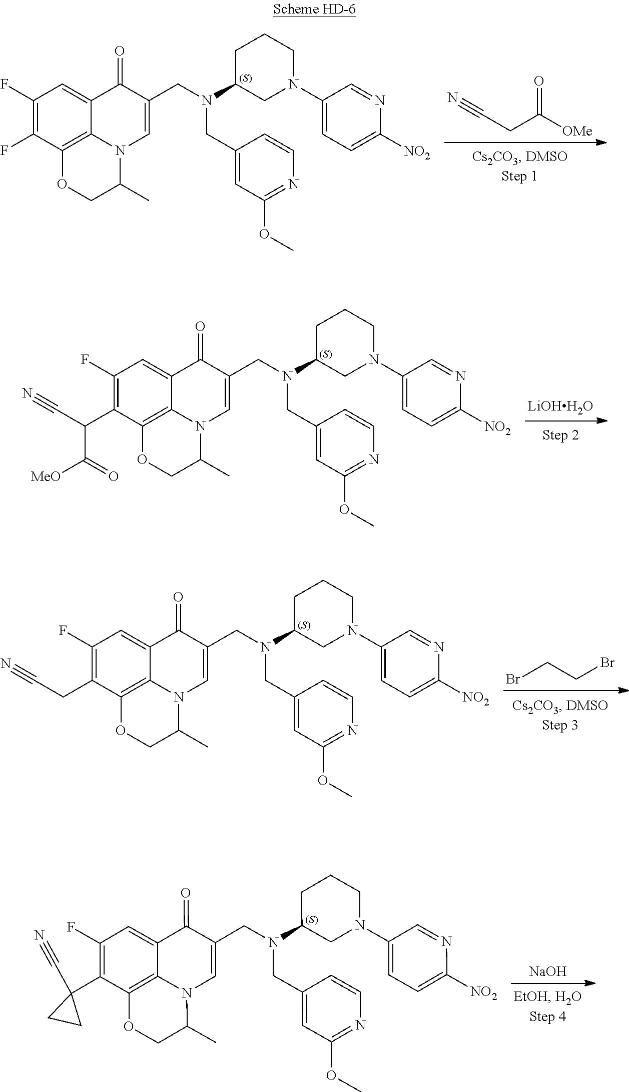

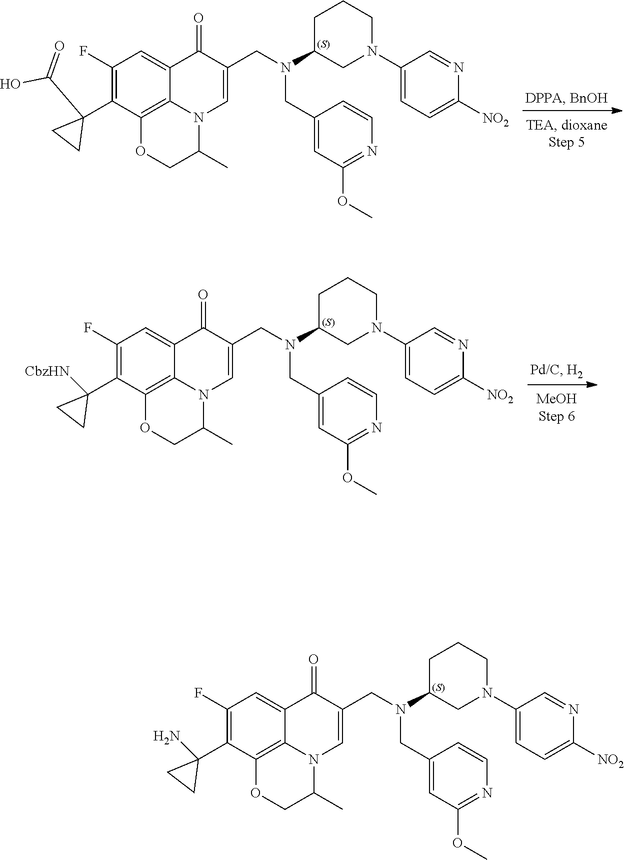

The compounds in Table S4 can be prepared according to Scheme HE-1 through Scheme HE-12.

TABLE S4

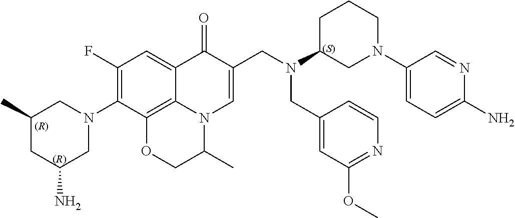

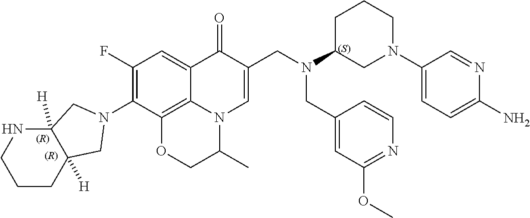

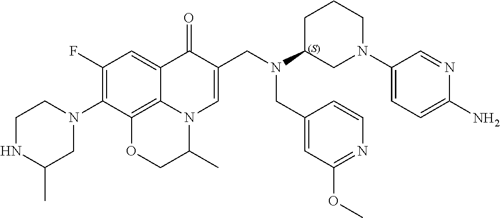

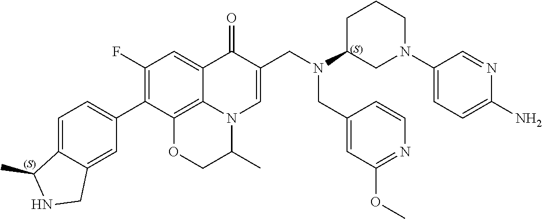

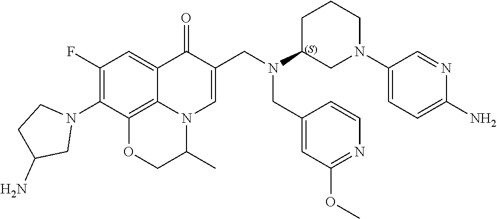

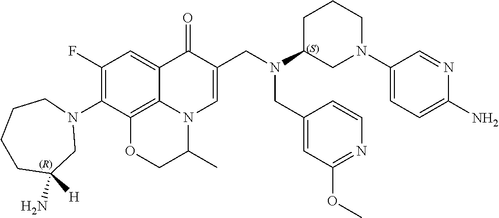

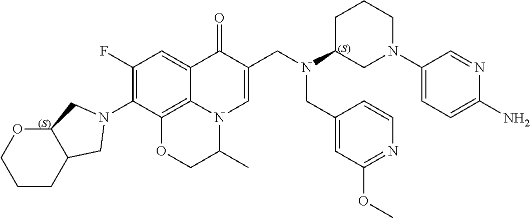

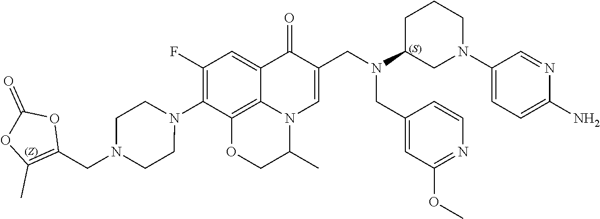

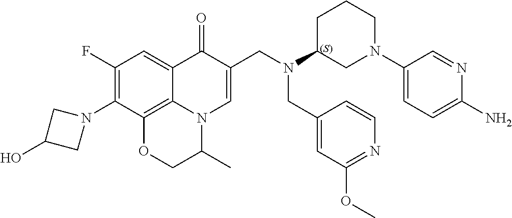

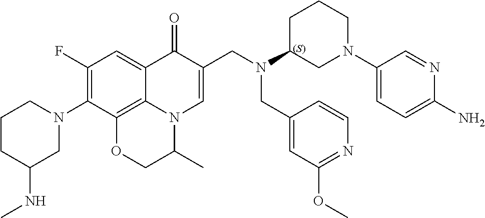

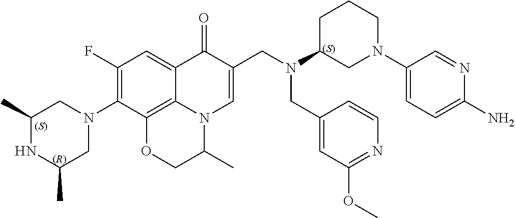

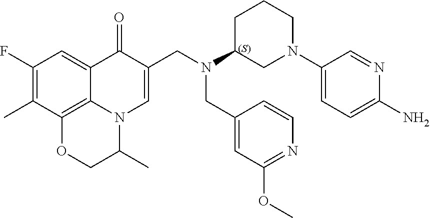

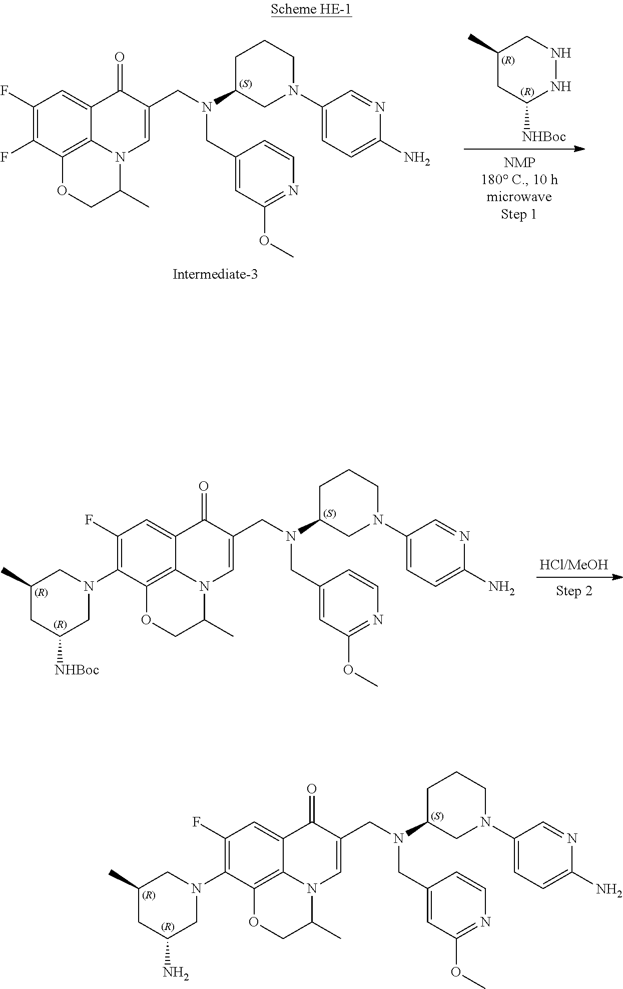

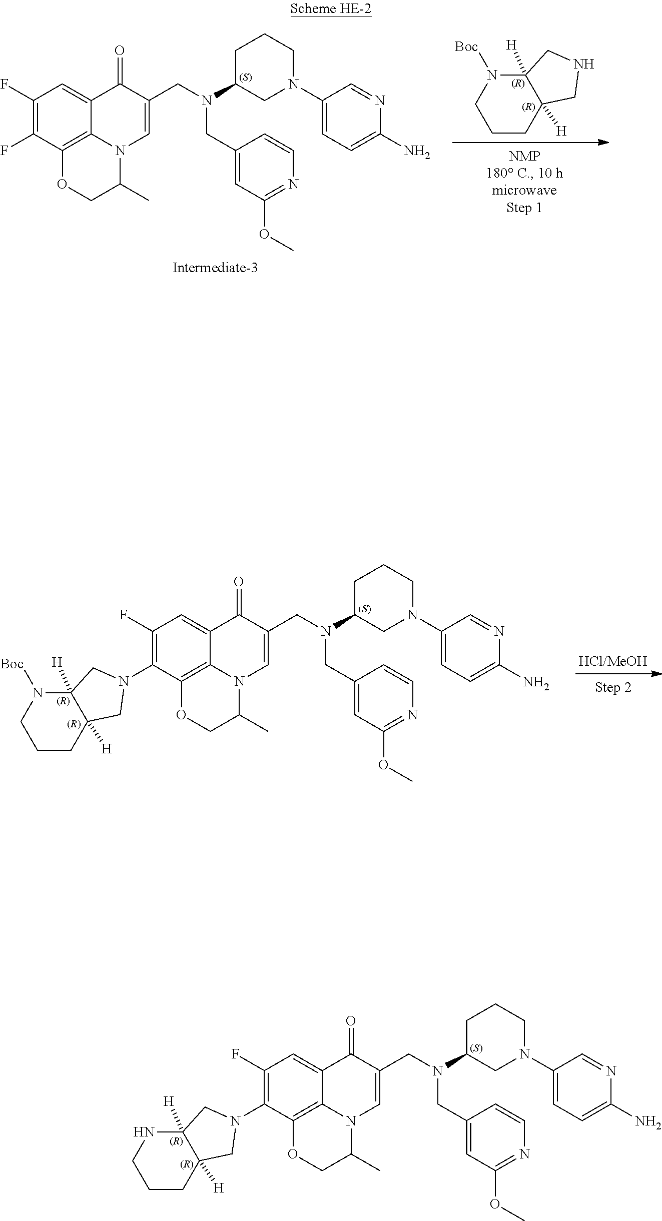

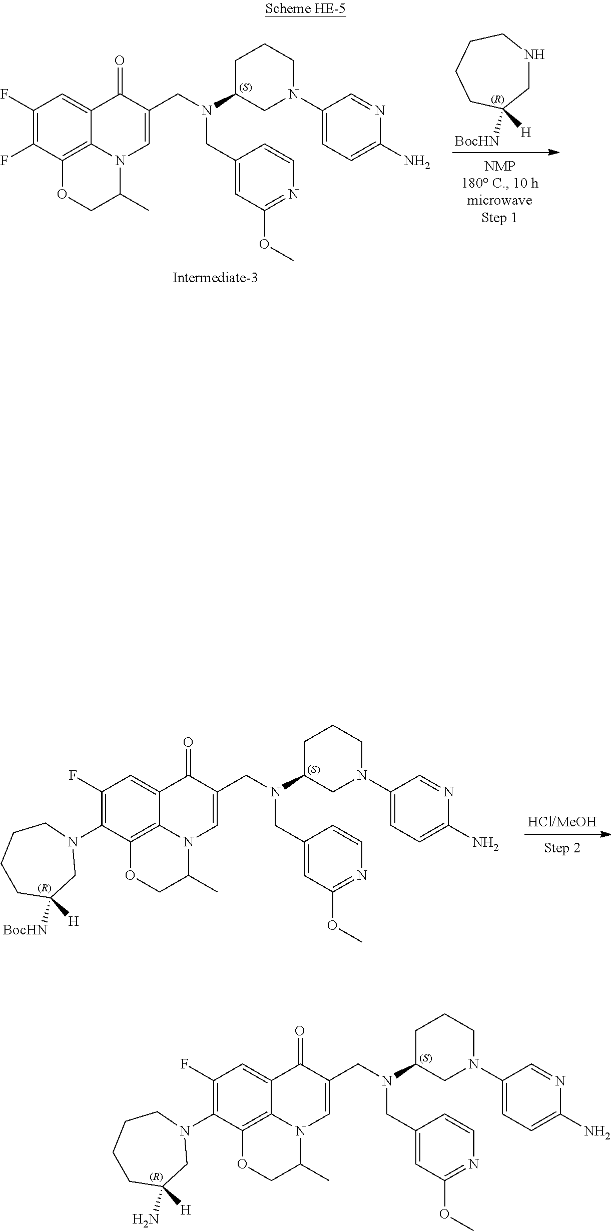

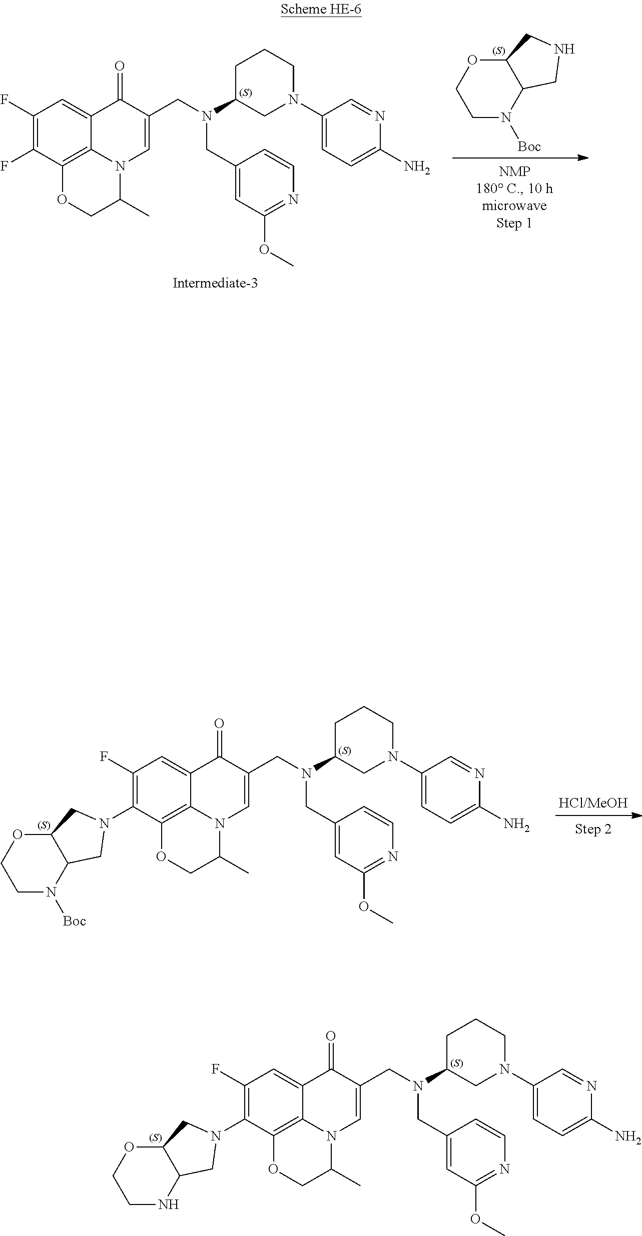

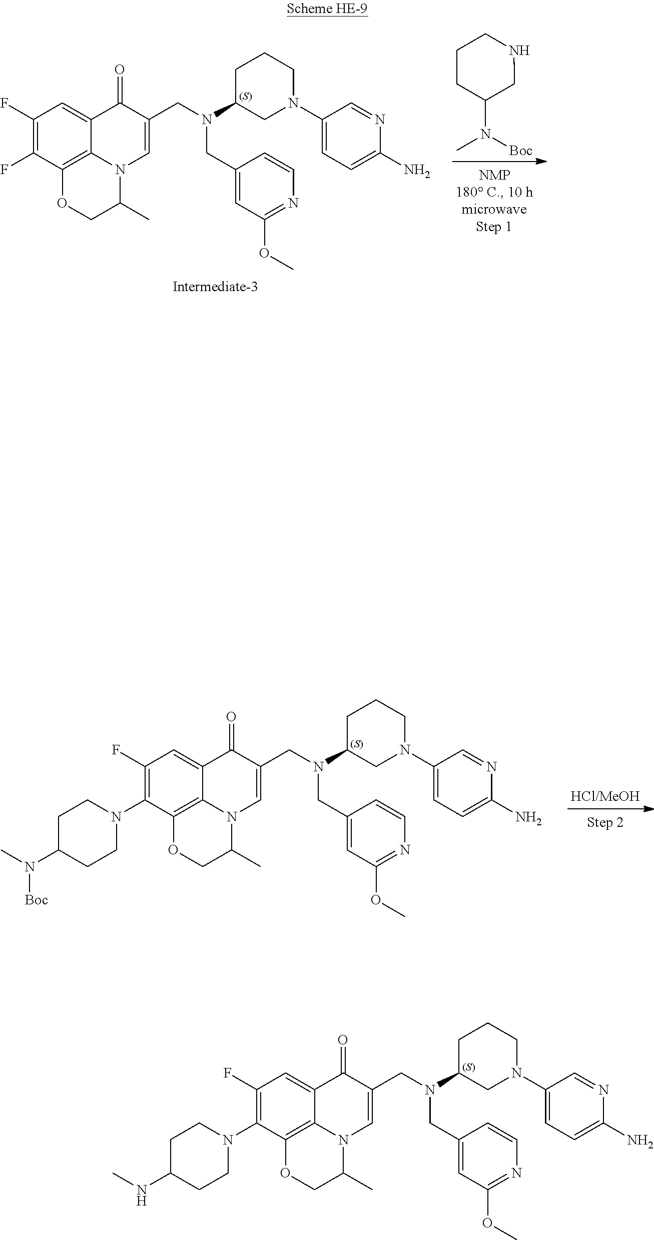

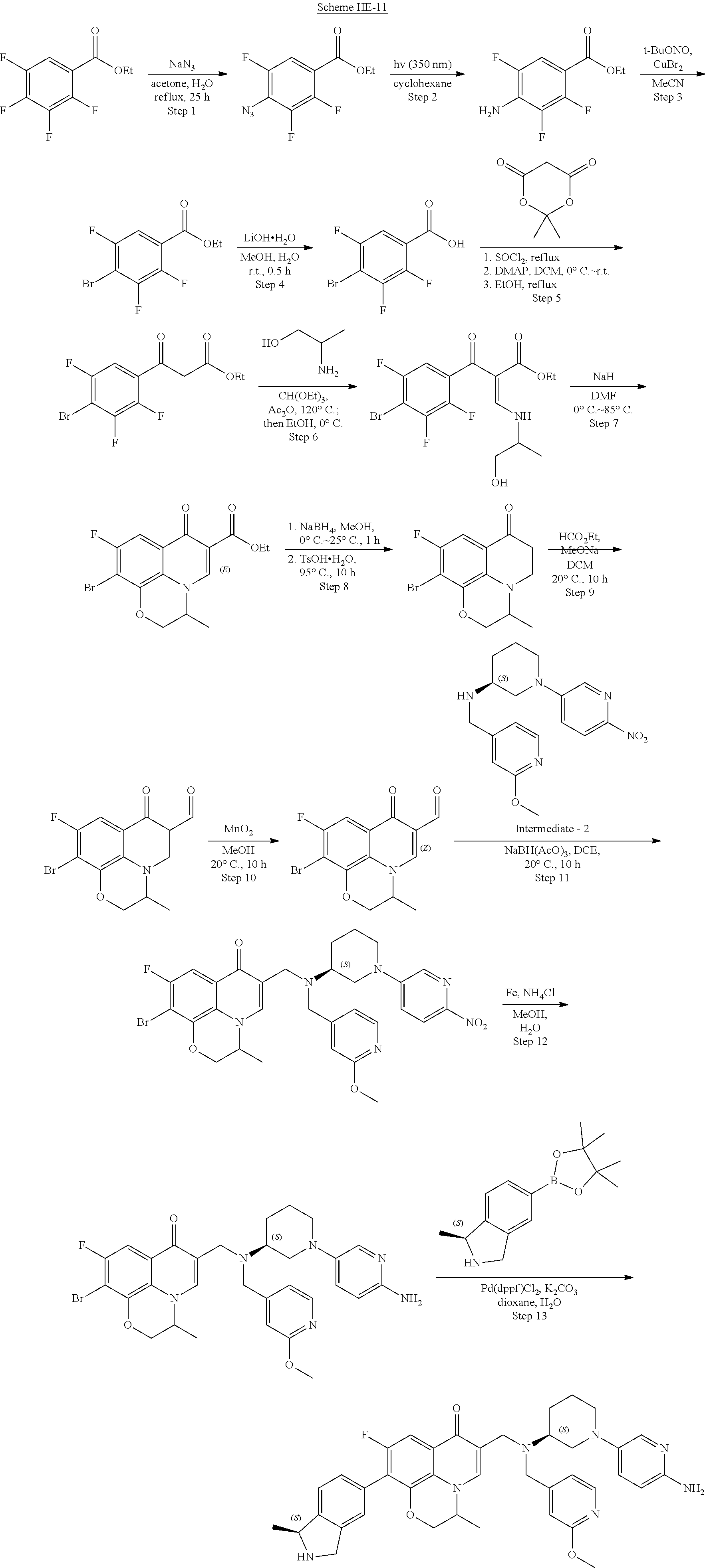

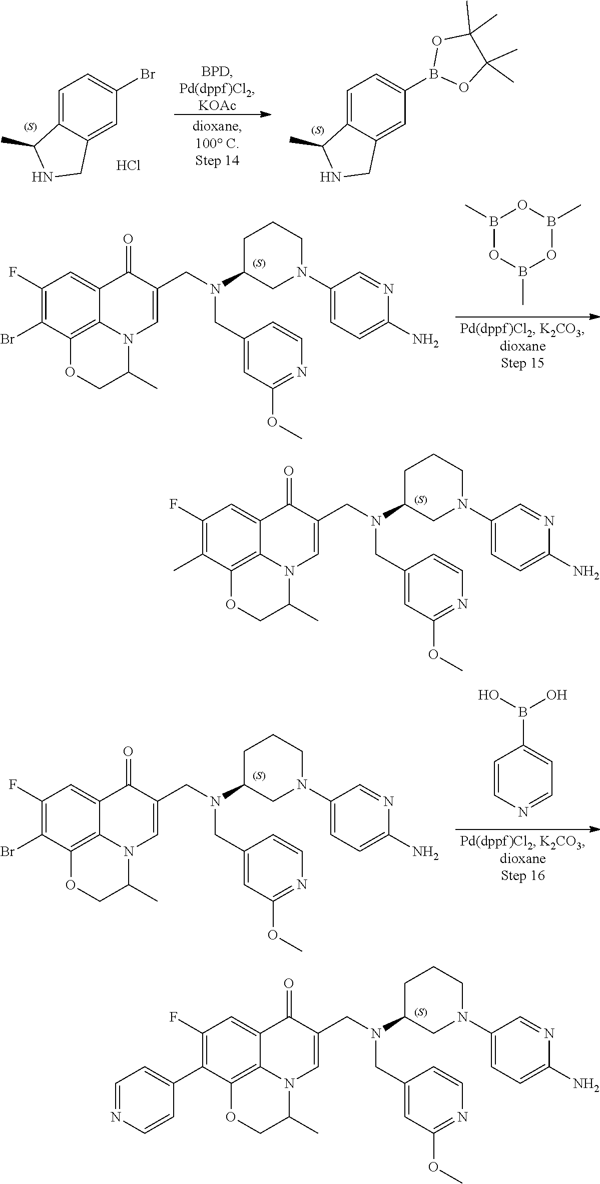

The compounds in Table S5 can be prepared according to Scheme HF-1 through Scheme HF-6.

TABLE S5

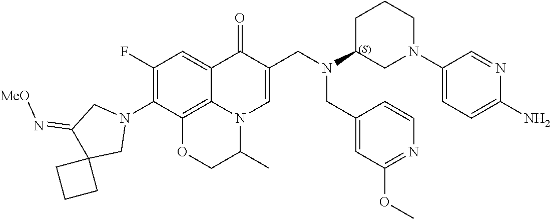

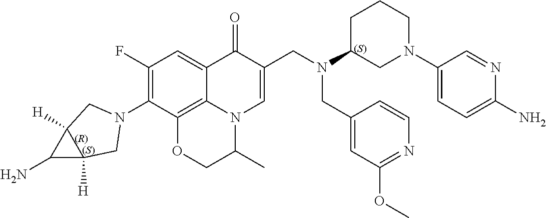

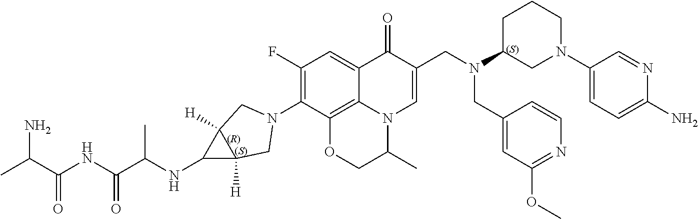

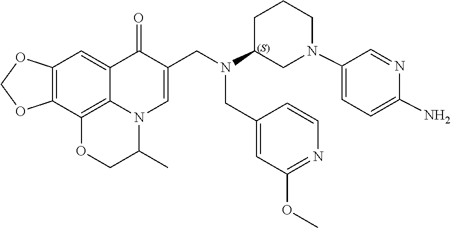

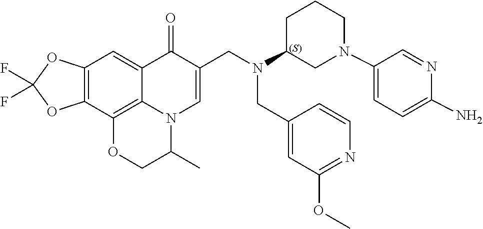

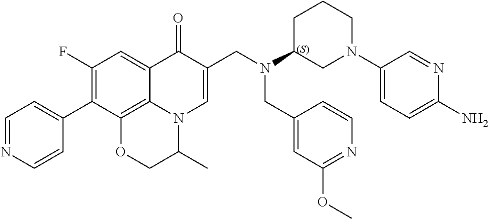

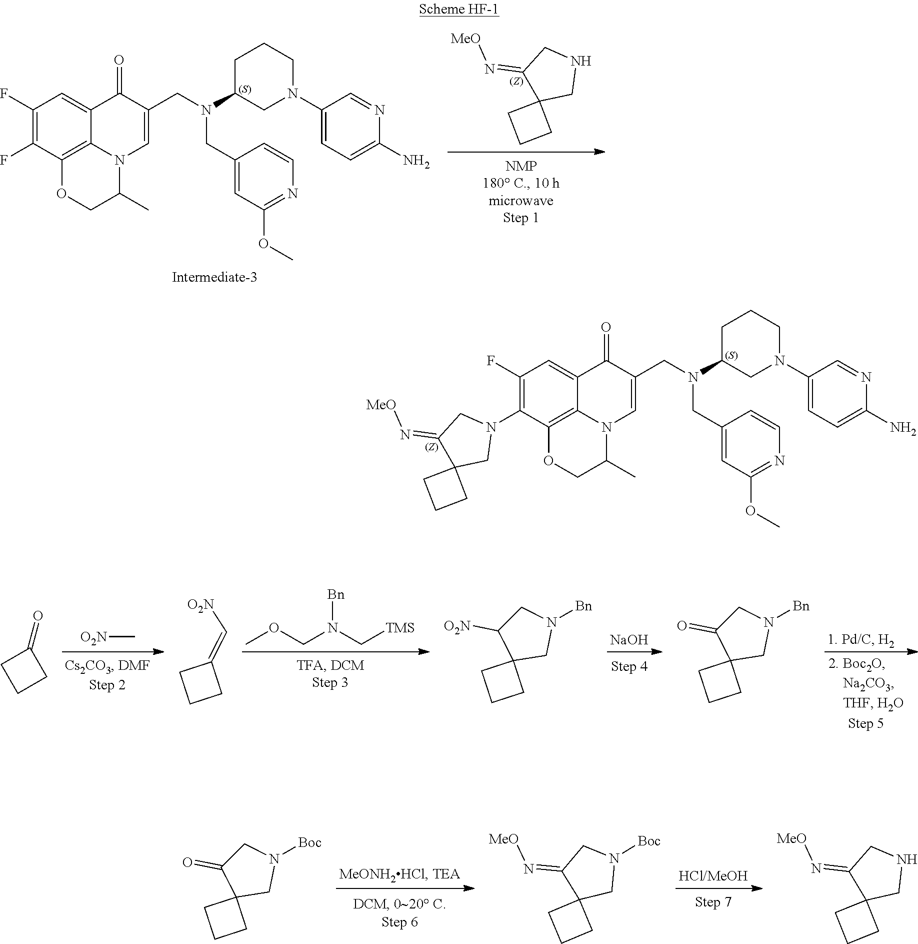

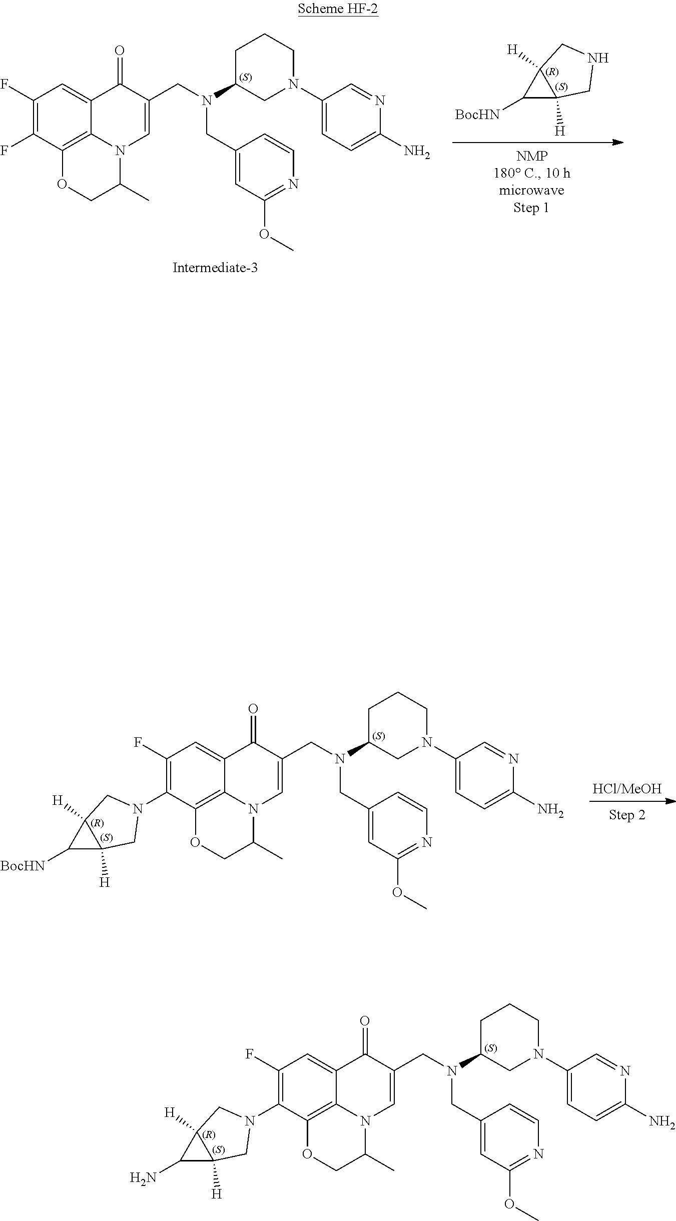

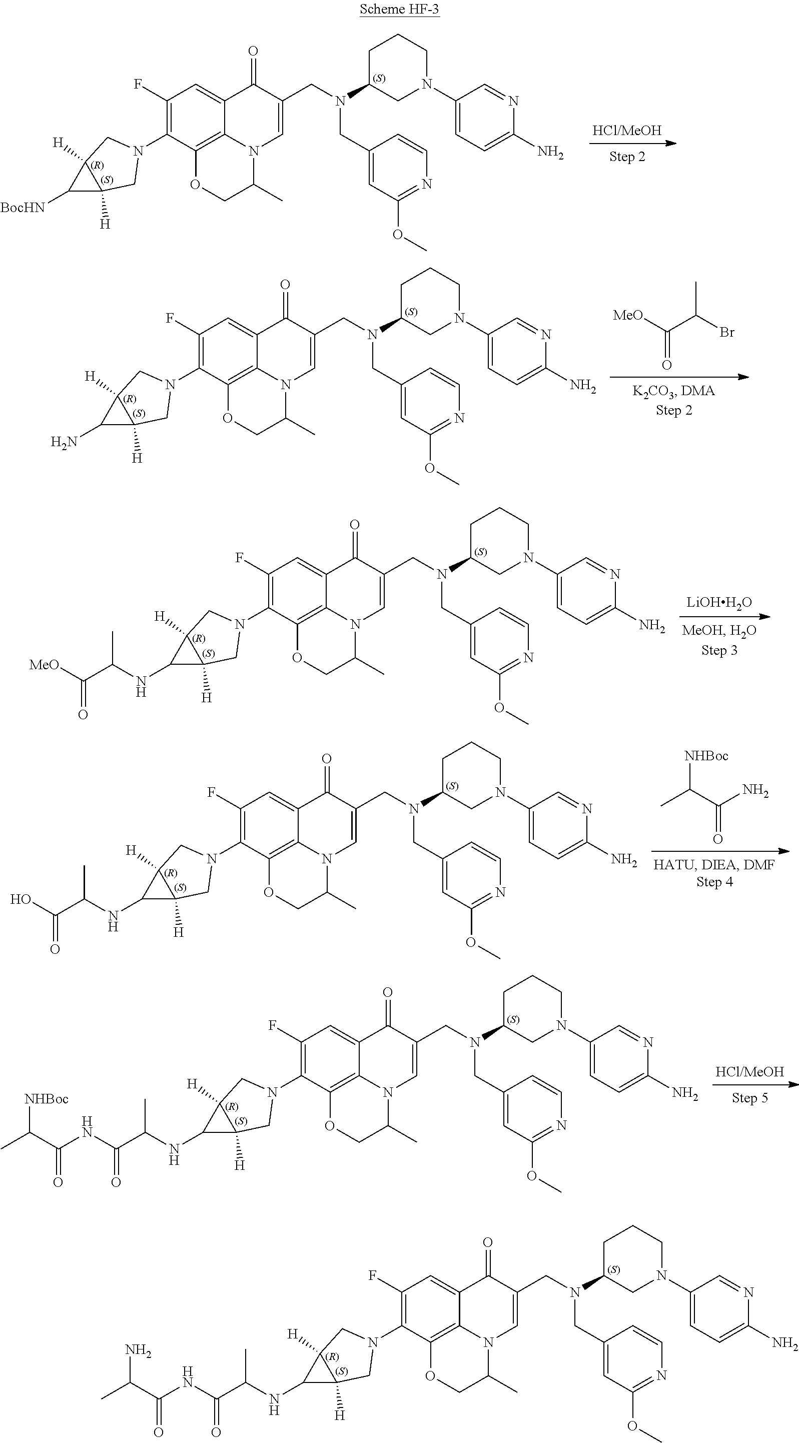

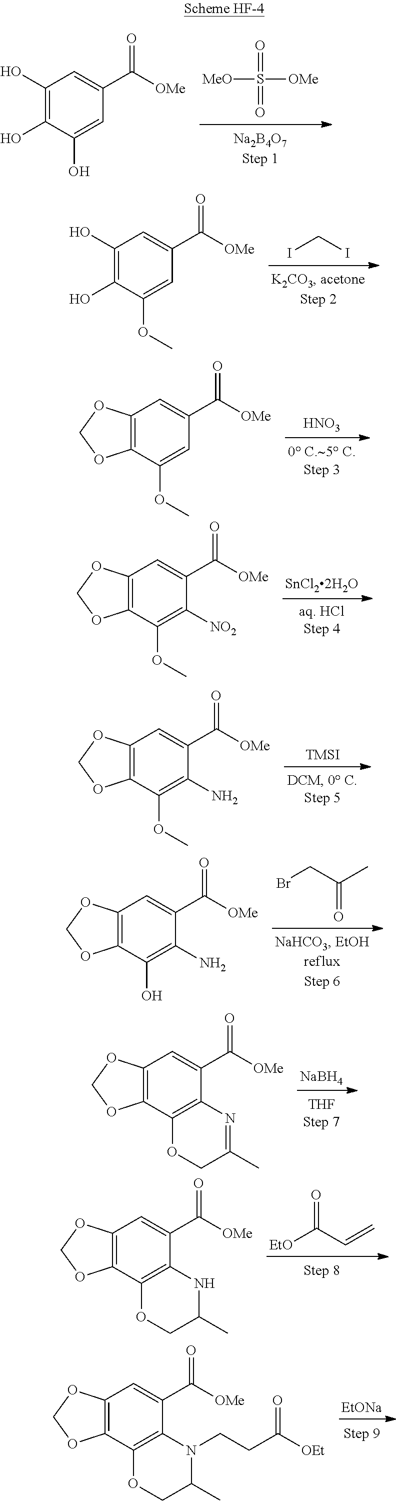

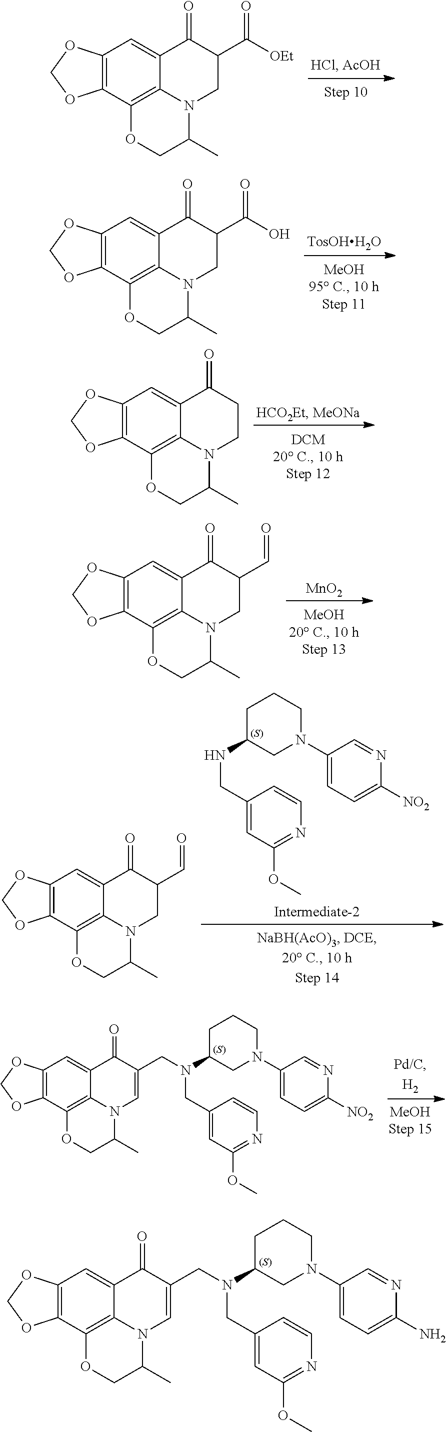

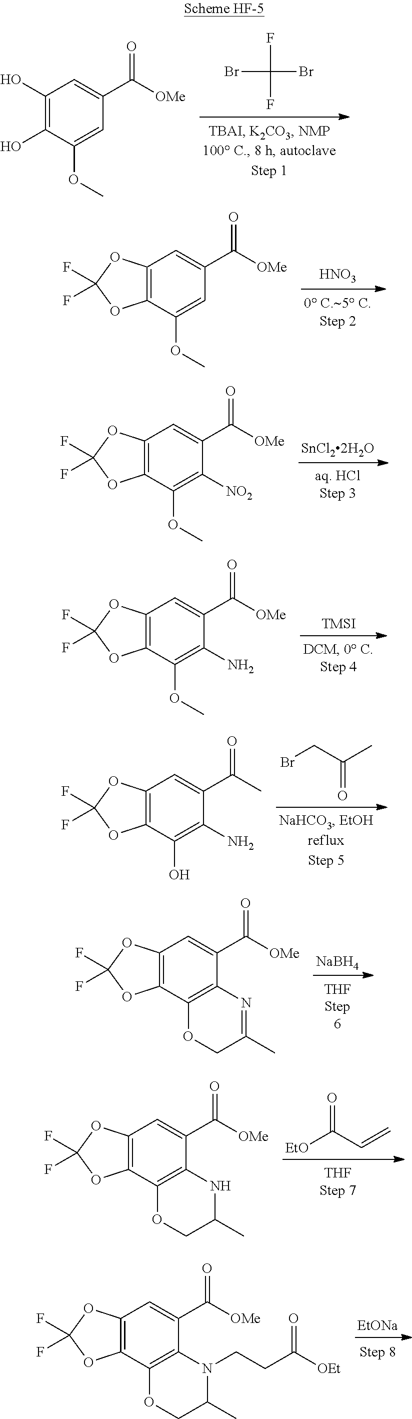

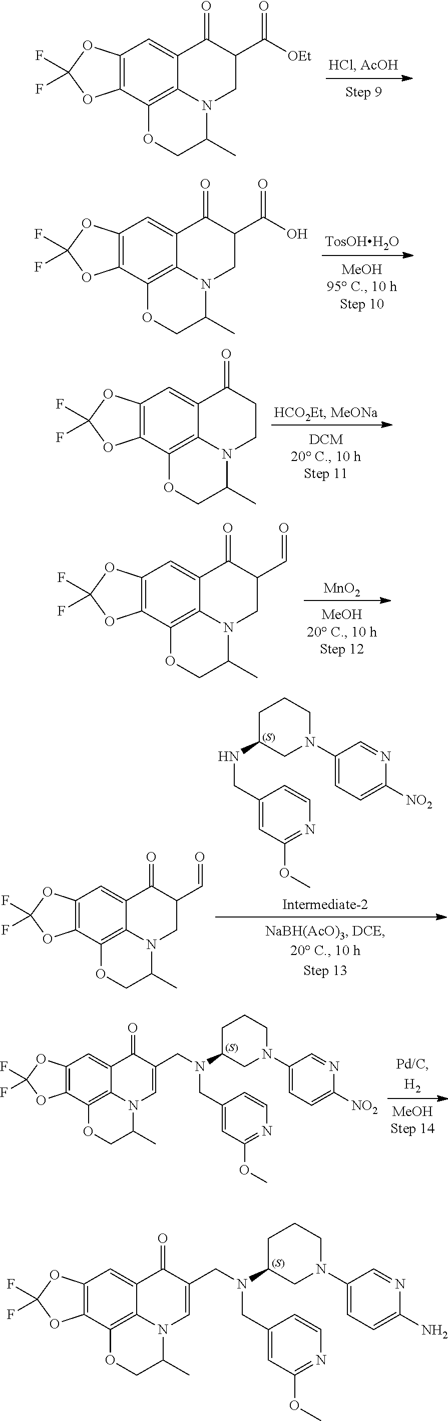

The compounds in Table S6 can be prepared according to Scheme HG-1 through Scheme HG-11.

TABLE S6

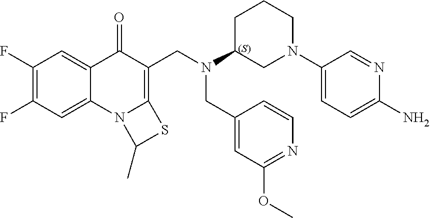

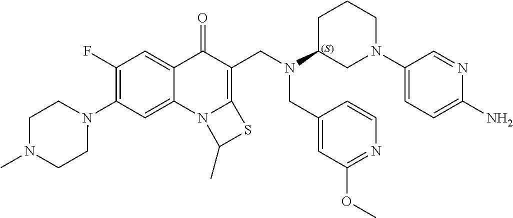

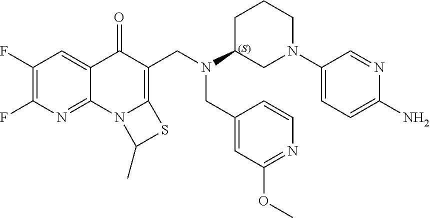

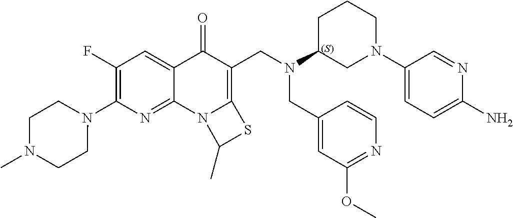

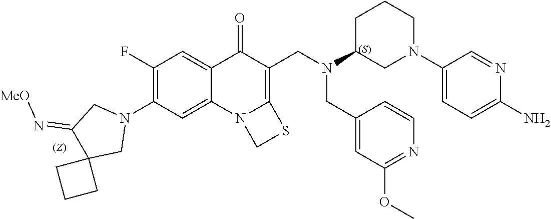

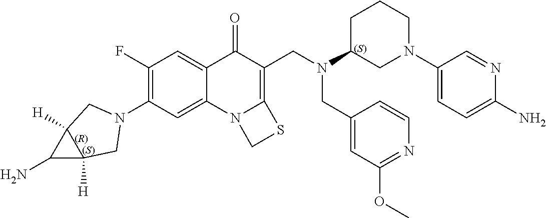

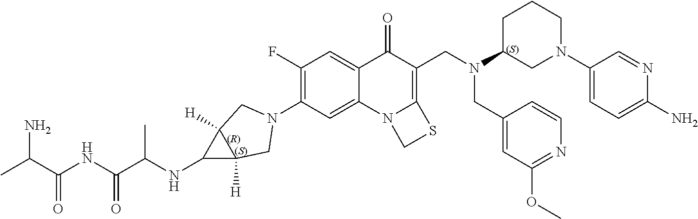

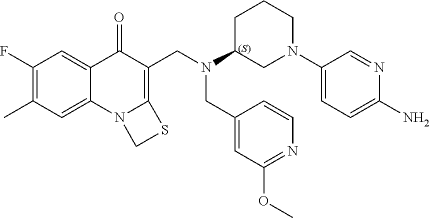

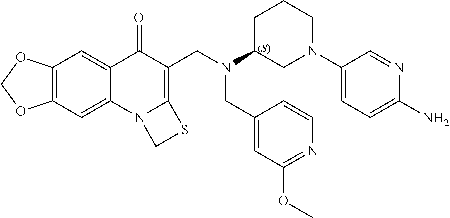

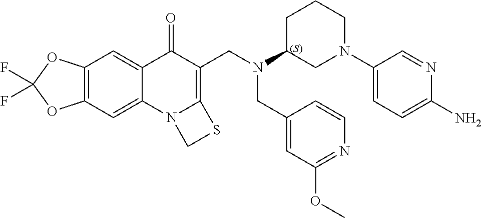

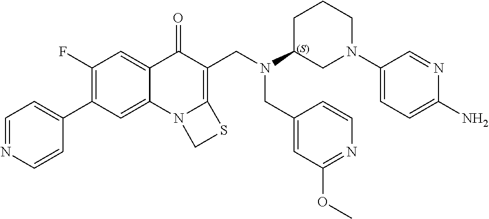

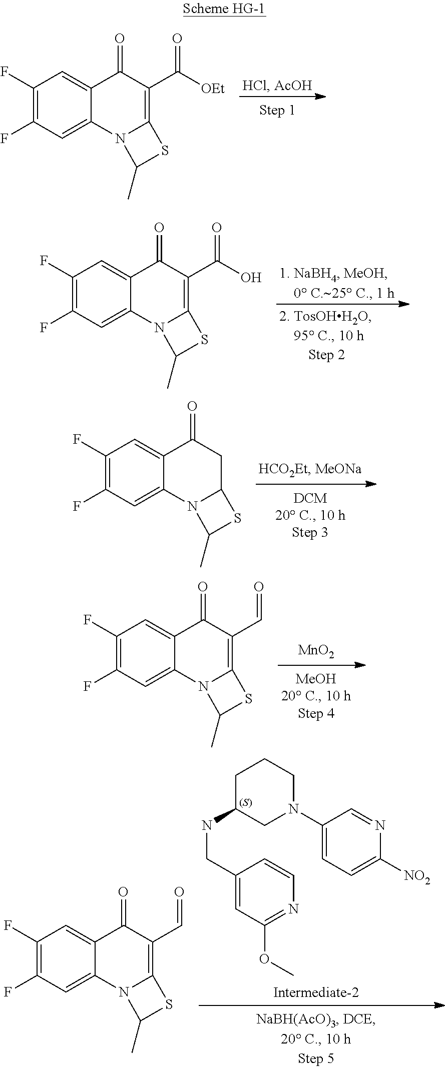

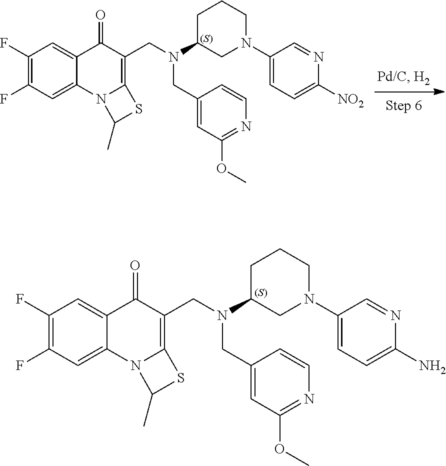

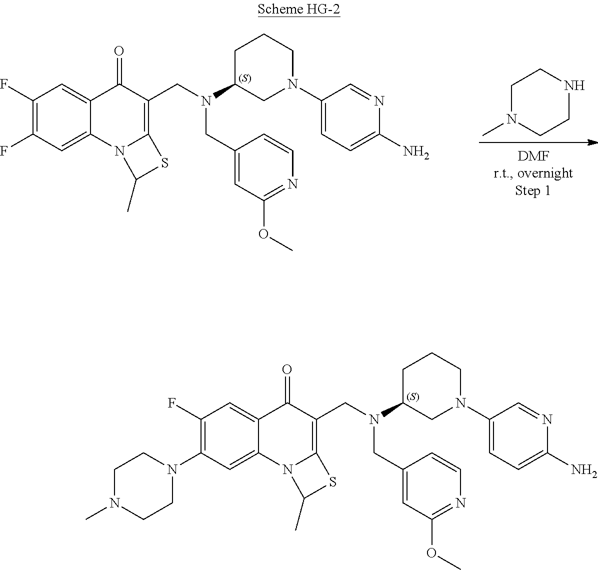

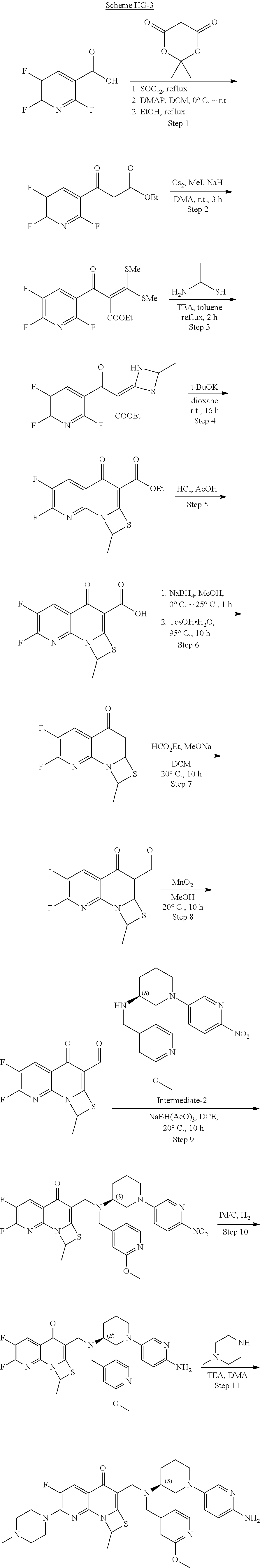

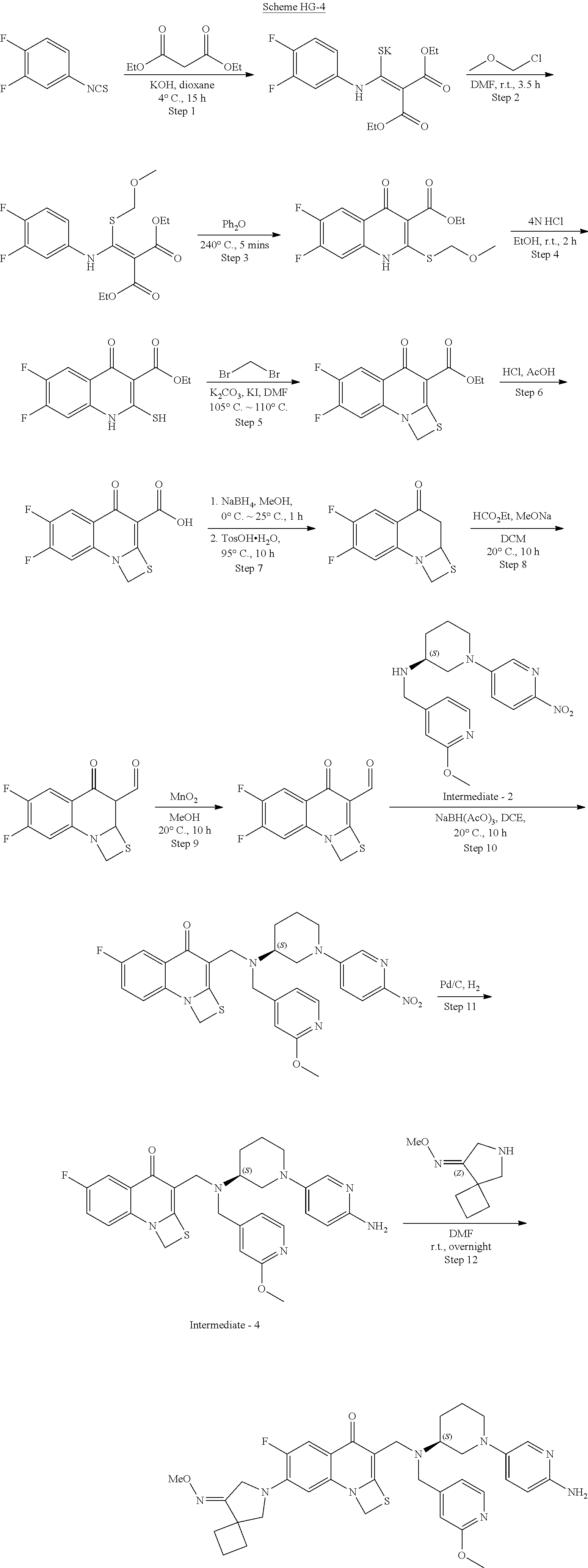

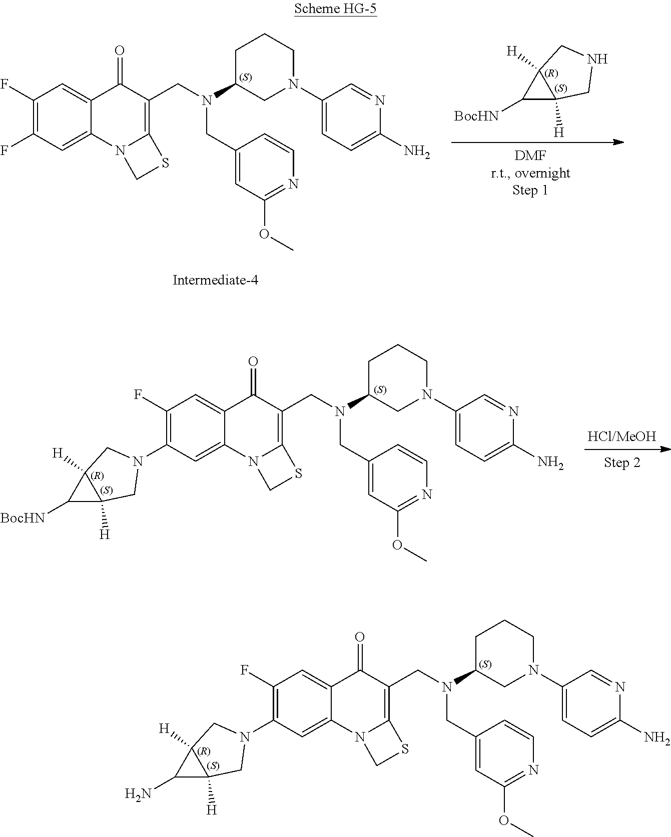

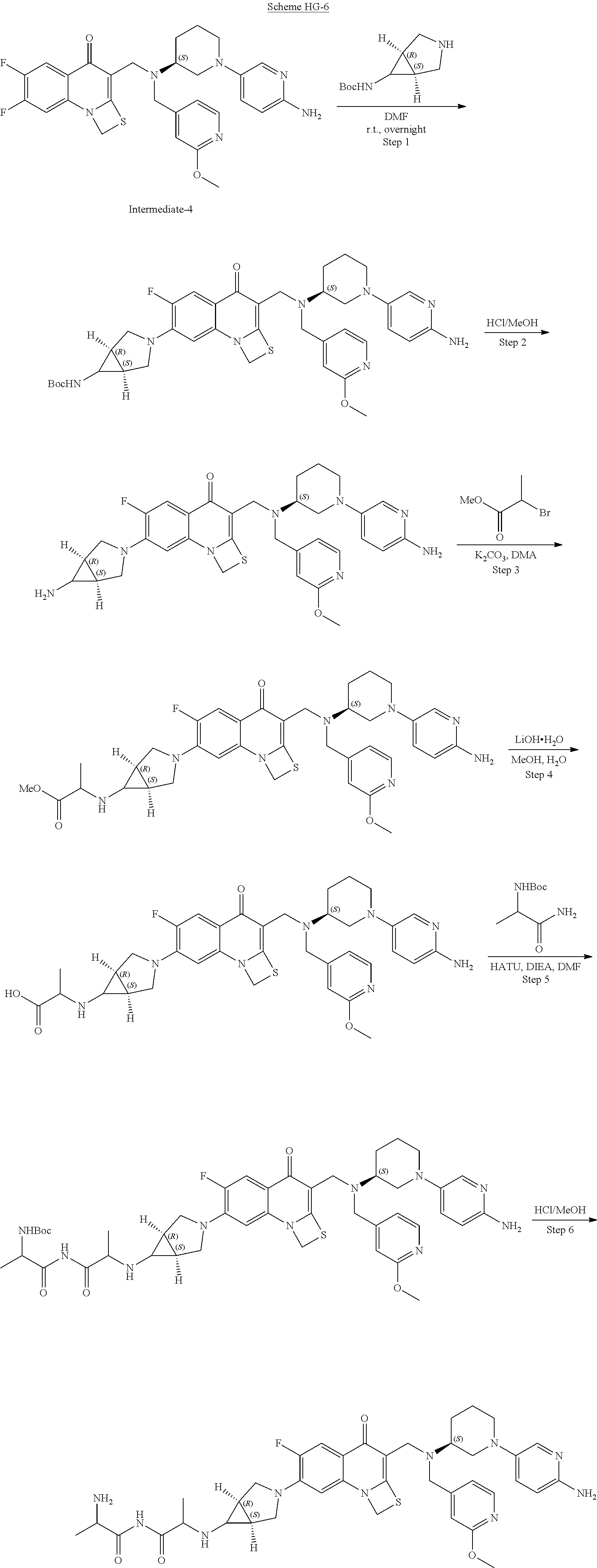

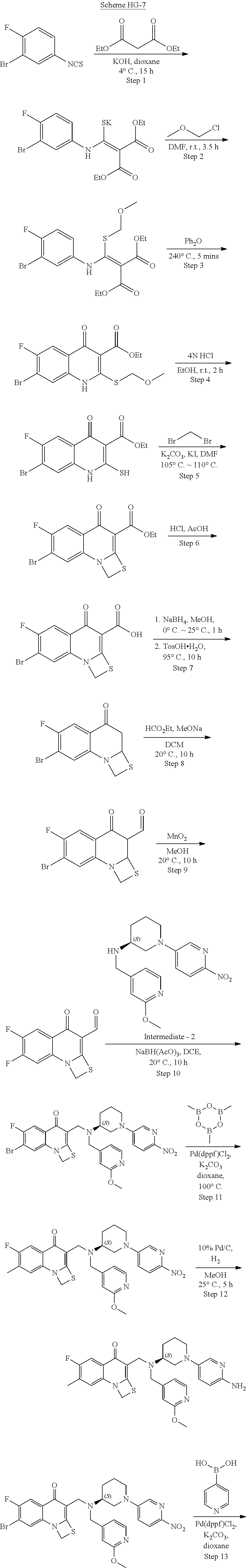

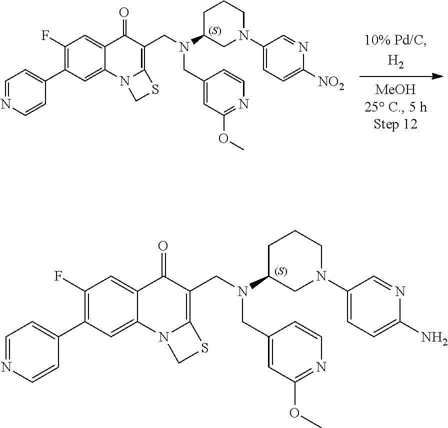

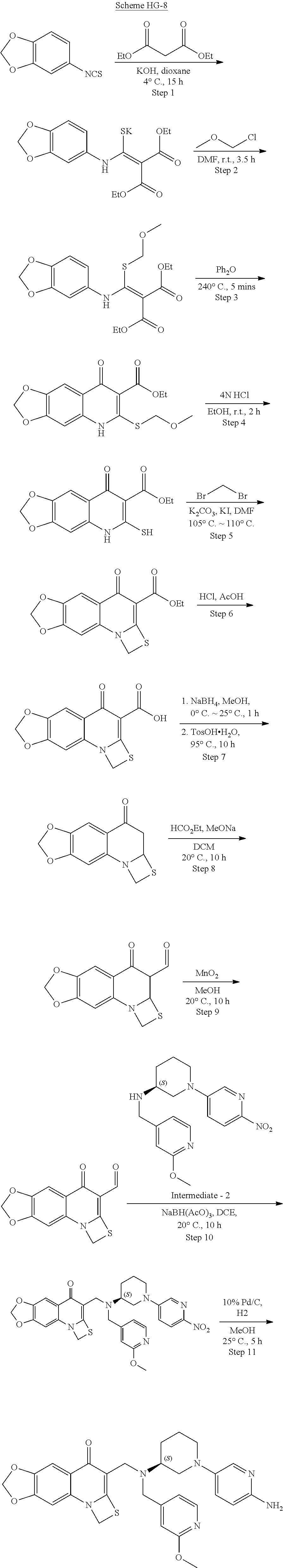

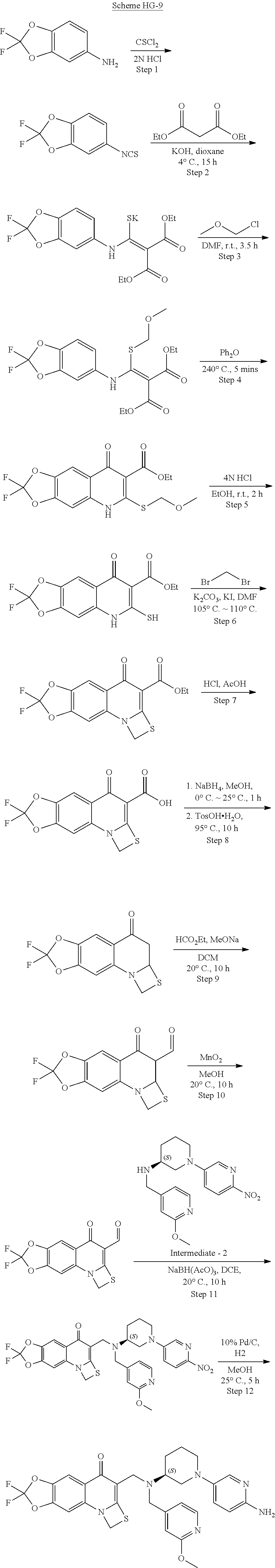

The compounds in Table S7 can be prepared according to Scheme HH-1 through Scheme HH-11.

TABLE S7

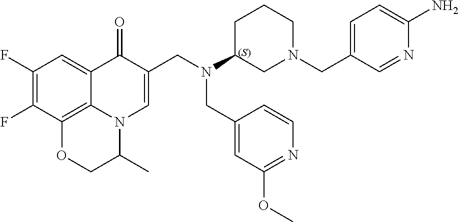

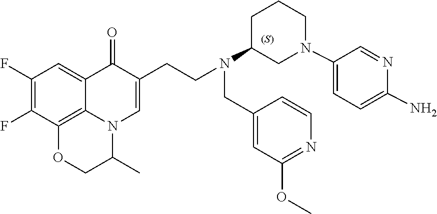

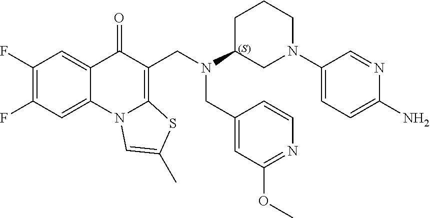

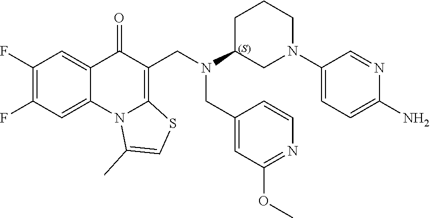

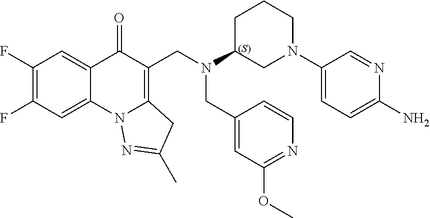

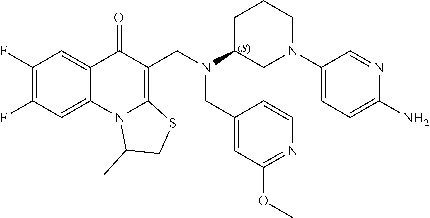

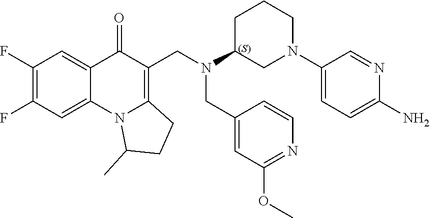

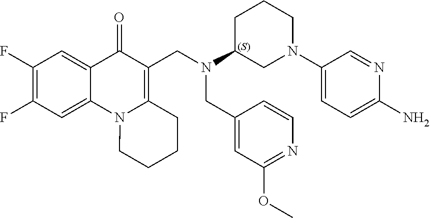

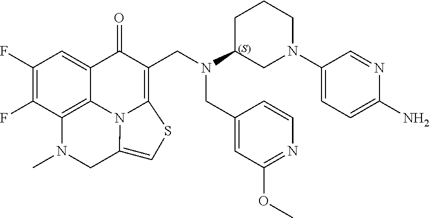

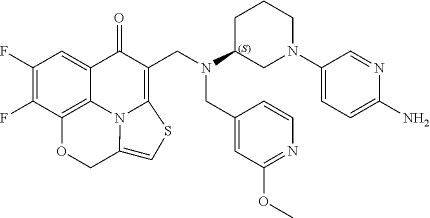

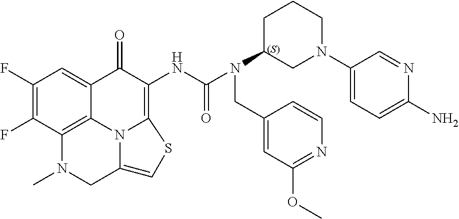

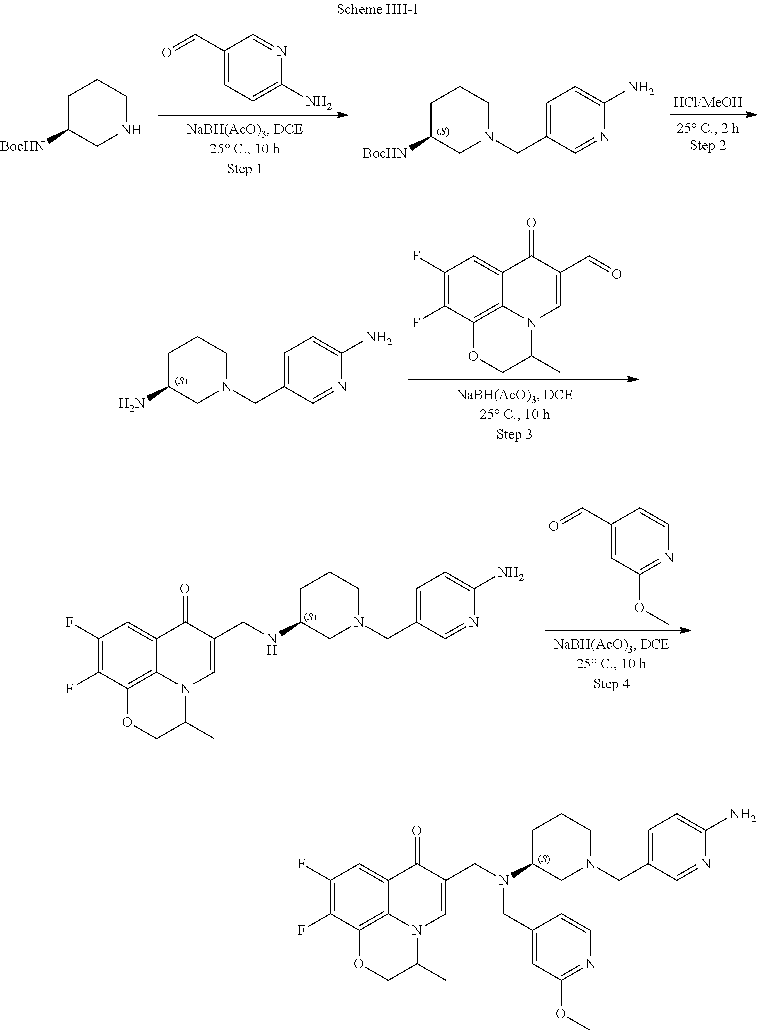

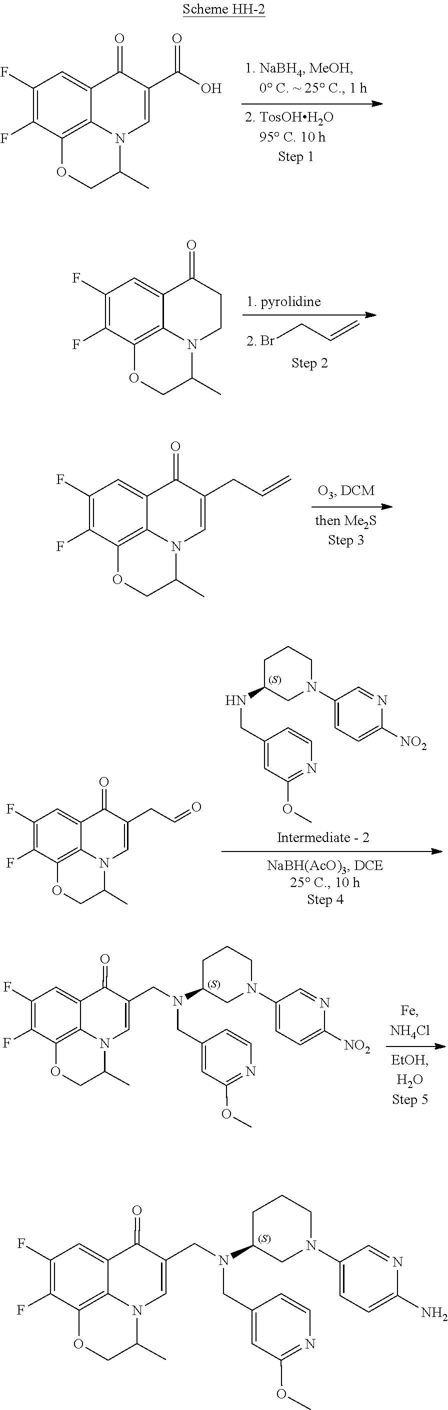

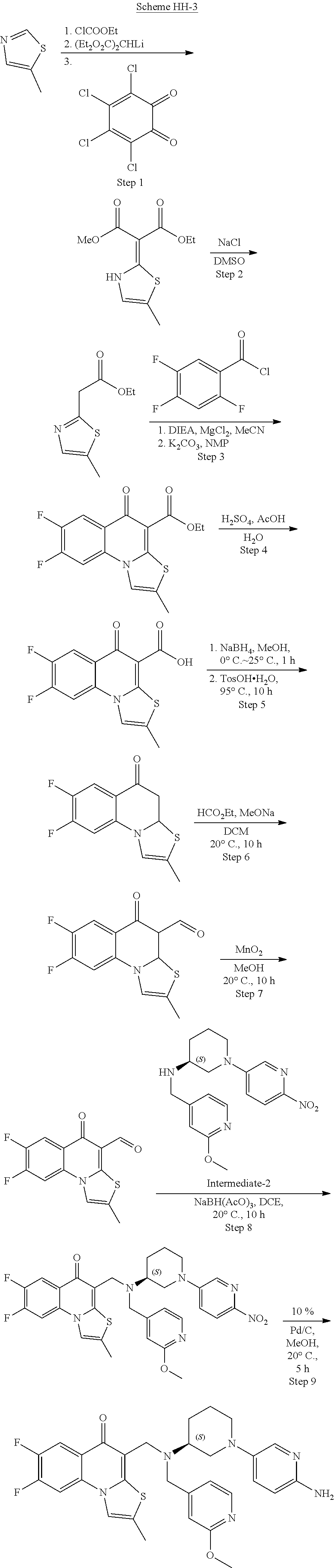

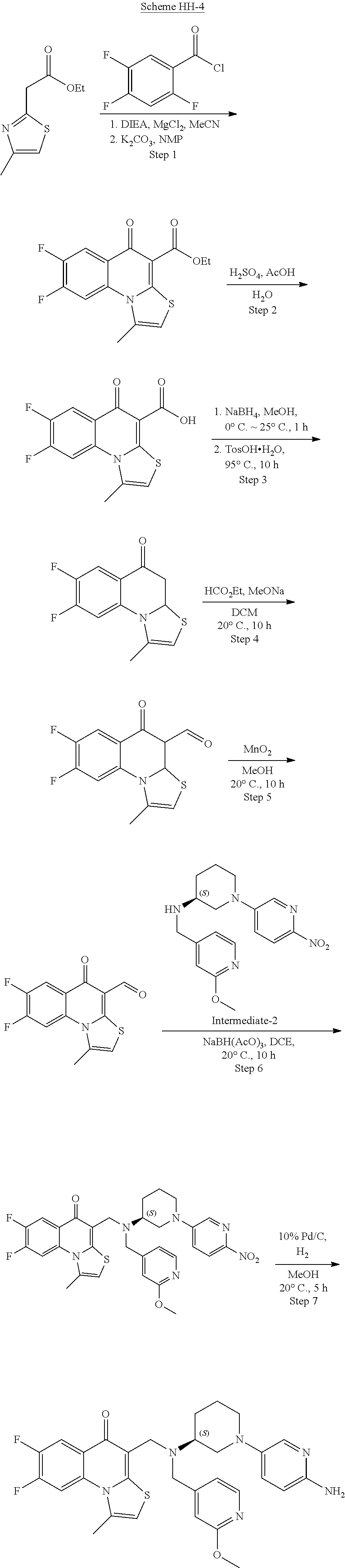

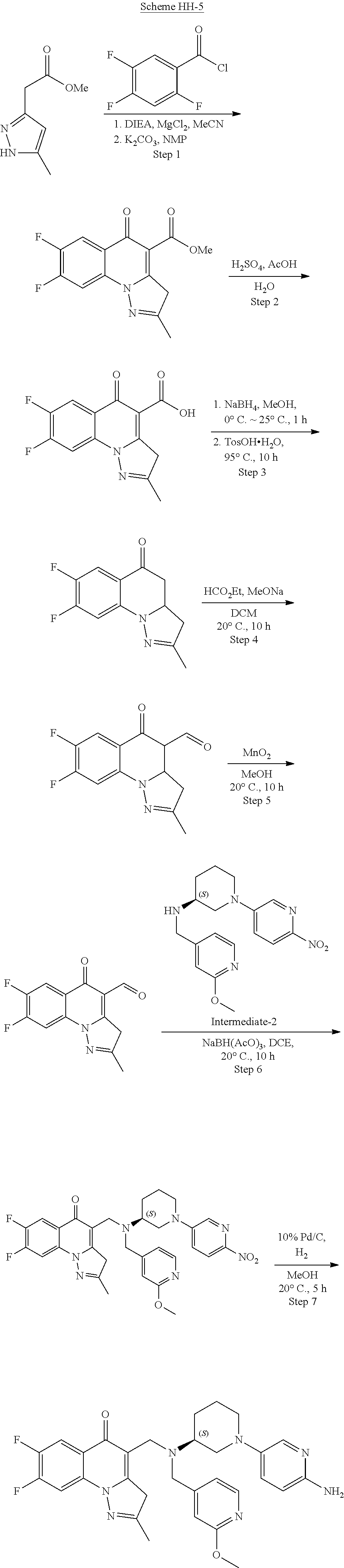

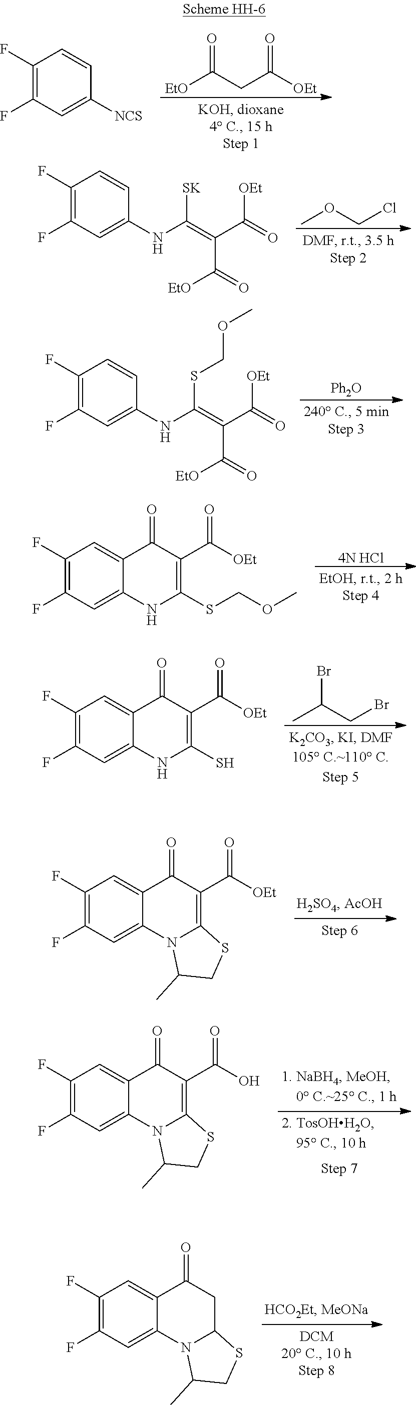

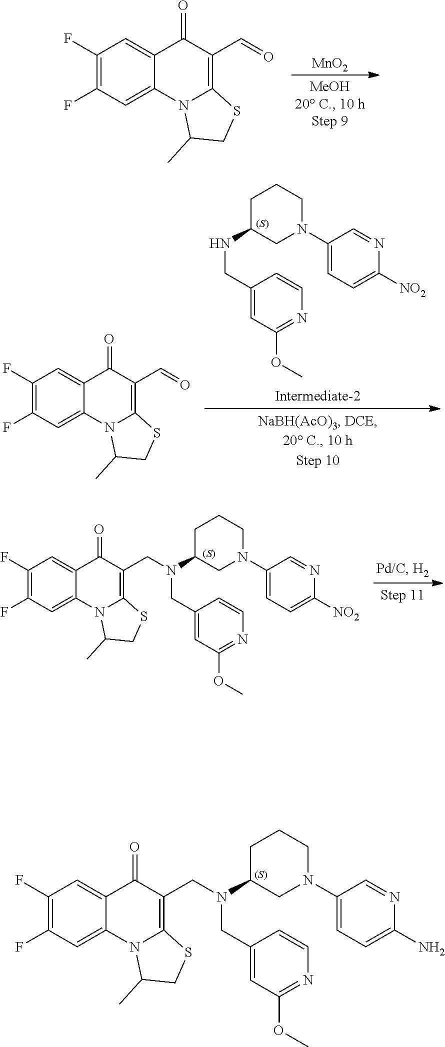

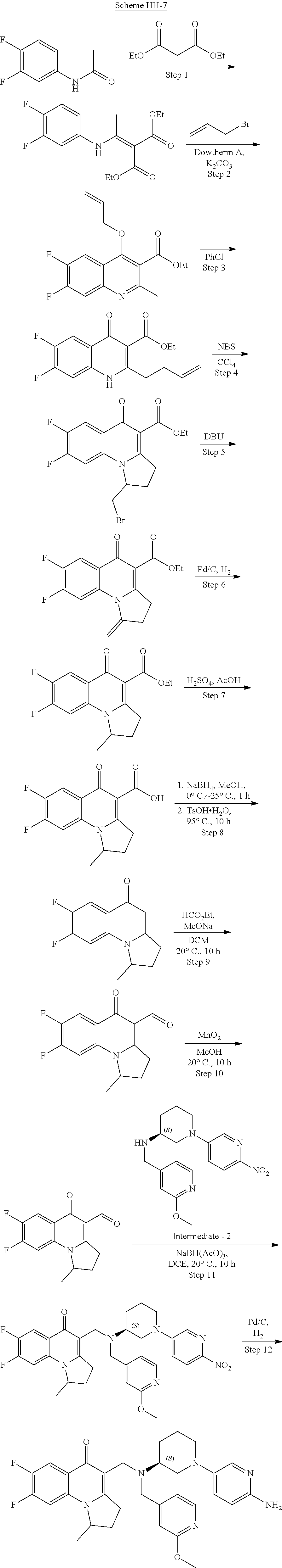

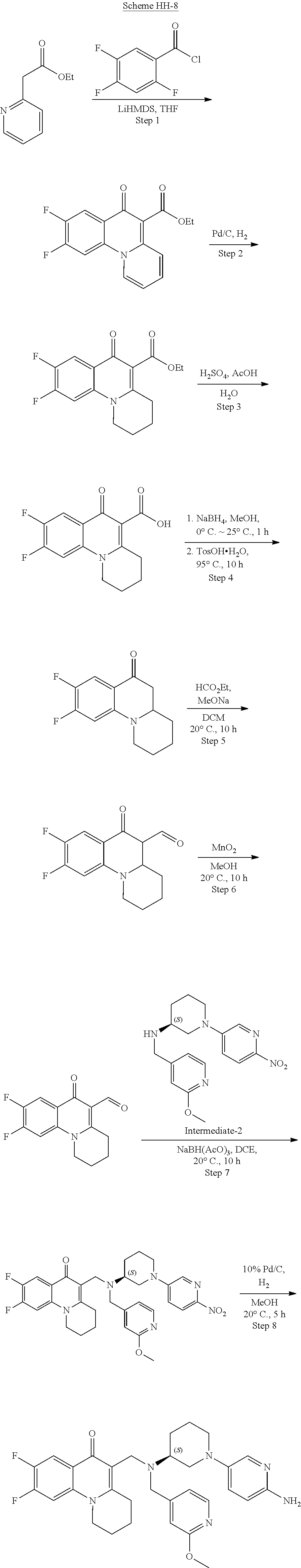

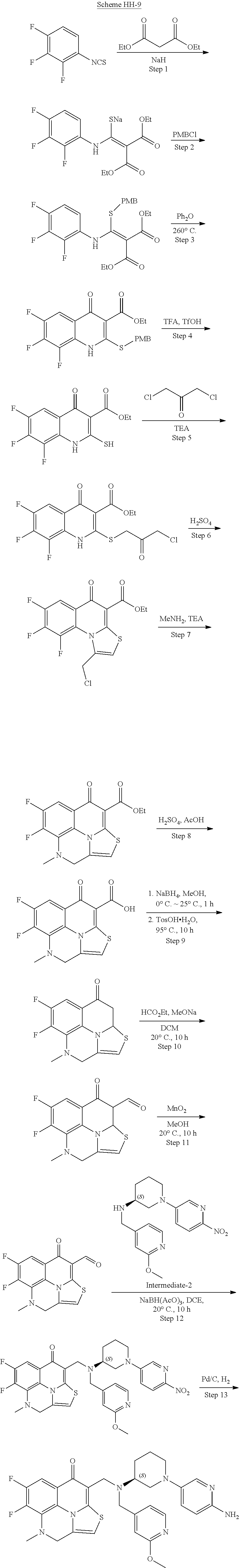

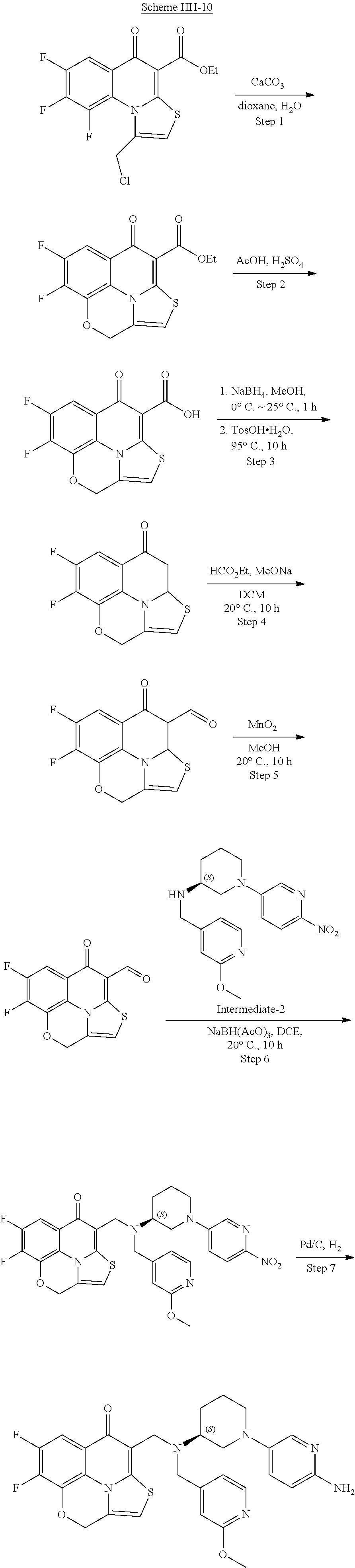

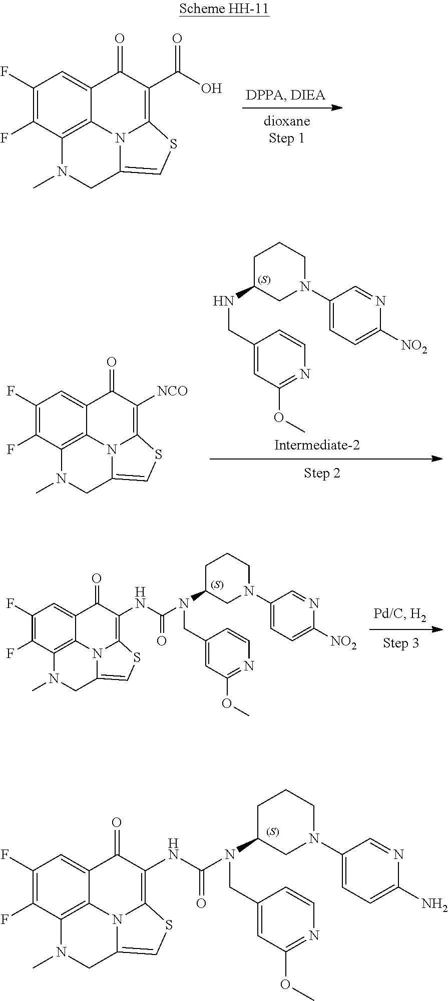

### Biological Assays

Cellular activity of STING agonists was determined by mRNA levels of IFN-β in THP1 (Otwo Biotech HTX1621) cell line. THP1 cells express the naturally occurring human HAQ STING isoform (hSTING HAQ), which responses to the activation of STING agonists.

THP1 cells were seeded into Falcon 12-well cell Culture Plate (Corning #353043) at 9×10⁵ cells per well, in 1 ml complete culture medium (RPMI-1640, GIBCO #C1187500BT) containing 10% heat inactivated FBS (GIBCO #10099141C), 1% Pen-Strep (HyClone SV30010) and incubated at 37° ΔC, 5% CO₂ overnight. On day 2, compound (final concentration at 3.6 μM) or DMSO (0.1%, Sigma Aldrich #8418) was added to THP1 cells and further incubated for 2 hrs min at 37° ΔC, 5% CO₂. Cells were then collected for RNA extraction.

Total RNA was extracted with TRIzol reagent (Invitrogen #15596026) and 1-5 g was used to synthesize first-strand cDNA with Maxima First Strand cDNA Synthesis Kit (TAKARA #RR036A). Quantitative PCR was performed with Maxima SYBR Green qPCR Master Mix (ABI #4367659, USA). The primer sequences used were as follows: GAPDH (5′-GTCAGCCGCATCTTCTTTTG-3′, 5′-GCGCCCAATACGACCAAATC-3′). INFP3 (5′-ATGACCAACAAGTGTCTCCTCC-3′,5′-GGAATCCAAGCAAGTTGTAGCTC-3′) Real-time PCR was performed on QuantStudio™ 5 Real-Time PCR System (Thermo Fisher). Relative expression levels of IFNβ gene were normalized against the level of GAPDH expression by the 2^{-ΔΔCT} method. Each PCR reaction was performed in triplicates.

Activities of mRNA INFβ and GAPDH

TABLE 1
Biological data

| | | Relative mRNA |
|---|---|---|
| | | INFβ/GAPDH |
| No. | Compound ID | THP 1 |
| | DMSO | 1.0 |
| 1 | Compound 1 | 447.9 |
| 2 | Compound 2 | 1261.9 |
| 3 | Compound 3 | 1356.3 |
| 4 | Compound 4 | 587.4 |
| 5 | Compound 5 | 1627.5 |
| 6 | Compound 6 | 1595.2 |
| 7 | Compound 7 | 65.3 |
| 8 | Compound 8 | 5.7 |
| 9 | Compound 9 | 332.6 |
| 10 | Compound 10 | 336.8 |
| 11 | Compound 11 | 65.6 |
| 12 | Compound 12 | 14.1 |
| 13 | Compound 13 | 3.6 |
| 14 | Compound 14 | 3848.9 |
| 15 | Compound 15 | 5.8 |
| 16 | Compound 16 | 114.6 |
| 17 | Compound 17 | 6.7 |
| 18 | Compound 18 | 4.8 |
| 19 | Compound 19 | 2625.9 |
| 20 | Compound 20 | 2142.1 |
| 21 | Compound 21 | 114.6 |
| 22 | Compound 22 | 6463.9 |
| 23 | Compound 24 | 437.3 |
| 24 | Compound 25 | 604.8 |
| 25 | Compound 26 | 3.5 |
| 26 | Compound 27 | 372.4 |
| 27 | Compound 28 | 1530.0 |
| 28 | Compound 29 | 3330.1 |
| 29 | Compound 30 | 986.1 |
| 30 | Compound 31 | 1162.4 |
| 31 | Compound 32 | 814.9 |
| 32 | Compound 33 | 849.2 |
| 33 | Compound 34 | 54.8 |
| 34 | Compound 35 | 3791.8 |
| 35 | Compound 36 | 9395.6 |
| 36 | Compound 37 | 6829.2 |
| 37 | Compound 38 | 672.0 |
| 38 | Compound 39 | 81.3 |
| 39 | Compound 40 | 352.8 |
| 40 | Compound 41 | 1.2 |
| 41 | Compound 42 | 12.1 |
| 42 | Compound 43 | 1.2 |
| 43 | Compound 44 | 642.4 |
| 44 | Compound 45 | 595.1 |
| 45 | Compound 46 | 691.4 |
| 46 | Compound 47 | 710.4 |
| 47 | Compound 48 | 1202.3 |
| 48 | Compound 49 | 5276.2 |
| 49 | Compound 50 | 6723.5 |
| 50 | Compound 51 | 7257.8 |
| 51 | Compound 52 | 178.2 |
| 52 | Compound 53 | 433.0 |
| 53 | Compound 53-P1 | 568.5 |
| 54 | Compound 53-P2 | 724.4 |
| 55 | Compound 54 | 3.9 |
| 56 | Compound 55 | 655.0 |
| 57 | Compound 56 | 366.2 |
| 58 | Compound 57 | 580.9 |
| 59 | Compound 58 | 688.4 |
| 60 | Compound 59 | 316.7 |
| 61 | Compound 60 | 174.3 |
| 62 | Compound 61 | 0.7 |
| 63 | Compound 62 | 915.2 |
| 64 | Compound 63 | 2.5 |
| 65 | Compound 64 | 26.6 |
| 66 | Compound 65 | 7.4 |
| 67 | Compound 66 | 7328.8 |
| 68 | Compound 67 | 7398.9 |
| 69 | Compound 69 | 9547.9 |
| 70 | Compound 70 | 6236.9 |
| 71 | Compound 71 | 6763.7 |
| 72 | Compound 72 | 31.9 |
| 73 | Compound 73 | 7.9 |
| 74 | Compound 74 | 1.4 |
| 75 | Compound 75 | 1.1 |
| 76 | Compound 76 | 0.8 |
| 77 | Compound 77 | 1.3 |
| 78 | Compound 78 | 1.2 |
| 79 | Compound 79 | 335.5 |
| 80 | Compound 80 | 6820.3 |
| 81 | Compound 81 | 5719.5 |
| 82 | Compound 82 | 349.0 |
| 83 | Compound 83 | 1.2 |
| 84 | Compound 84 | 0.9 |
| 85 | Compound 85 | 0.9 |
| 86 | Compound 86 | 1.1 |
| 87 | Compound 87 | 1.2 |
| 88 | Compound 88 | 0.8 |
| 89 | Compound 89 | 1.3 |
| 90 | Compound 90 | 1.7 |
| 91 | Compound 91 | 1.0 |
| 92 | Compound 92 | 1.1 |
| 93 | Compound 93 | 411.3 |
| 94 | Compound 94 | 248.8 |
| 95 | Compound 95 | 1.2 |
| 96 | Compound 96 | 451.1 |
| 97 | Compound 97 | 1.0 |
| 98 | Compound 98 | 301.6 |
| 99 | Compound 99 | 368.7 |
| 100 | Compound 100 | 581.3 |
| 101 | Compound 101 | 332.9 |
| 102 | Compound 102 | 0.9 |
| 103 | Compound 103 | 356.0 |

hERG Safety Evaluation

### 1. Cell Lines and Cell Culture:

(1) hERG stably expressed HEK 293 cell line (#60187) was purchased from BPS Bioscience and P3-P20 were used for the study. Cells were cultured in medium containing: 90% MEM, 10% FBS, 0.1 mM NEAA, 100 U/mL Penicillin-Streptomycin, 1 mM Na pyruvate and 500 μg/mL Geneticin. Cells are split about three times a week, and maintained between ˜40% to ˜80% confluence. 24 to 48 hours prior to recordings, the cells were plated on 0.05 mg/mL PDL pre-coated glass cover slips placed in 48-well plate at 1×10⁴ cells.

(2) The number of cells being plated on the cover slip should reach a confluence rate at which majority of the cells are single.

### 2. Working Solution Preparation for Test Compound:

(1) Test compounds were initially prepared in DMSO with final concentration of 10 mM as stock solution.

(2) Compound was first made into 10, 3, 1, 0.3 and 0.1 mM DMSO stock, then serial diluted into the desired test concentrations in the extracellular solution (ECs). 30 uM test solution was diluted from 10 mM DMSO stock. The final DMSO concentration was in range of 0.1-0.3%.

### 3. Manual Whole Cell Patch and Recording Procedures:

HEKA EPC 10 USB patch clamp amplifier (from HEKA Elektronik, Germany) was used in the whole cell recording. A cover slip with plenty of single HEK293 hERG cells on the surface was removed and placed into a continuously perfused (approximately 1 ml/minute) recording chamber mounted on an inverted microscope. HERG channel currents were recorded from single cells using standard whole cell recording techniques. The cells were voltage clamped at a holding potential of −80 mV. Then hERG current was activated by depolarizing at +30 mV for 5000 ms, after which the current was taken back to −50 mV for 5000 ms to remove the inactivation and observe the deactivating tail current. The K+ tail current through HERG channels observed during this step was allowed to stabilize under continuous bath perfusion. Cells were then superfused with drug until steady state block was achieved. Steady state was considered reached when three consecutive super-imposable current records were collected. At this point, cells were once again superfused with extracellular solution until the current amplitude returned to values close to those measured before application of drug.

### 4. Data Analysis:

(1) Percent current inhibition was calculated using the following equation. (Note: PatchMaster was used to extract the peak current from the original data.)

$$normalize value ( \% of hERG inhibition ) = ( Saline current - concentration current ) / ( Saline current ) * 100$$

(2) The dose response curve of test compounds was plotted with % inhibition against the concentration of test compounds using Graphpad Prism 9.0, and fit the data to a log(inhibitor) vs. response—Variable slope curve.

TABLE 2
Summary of hERG IC₅₀ values

| No | cpd number | hERG IC₅₀ (μM) |
|---|---|---|
| 1 | RVU-25466 | 0.35 |
| 2 | Compound 1 | 1.58 |
| 3 | Compound 29 | 34.1 |
| 4 | Compound 53 | 1.27 |
| 5 | Compound 66 | 2.72 |
| 6 | Compound 67 | 3.08 |
| 7 | Compound 69 | 1.94 |
| 8 | Compound 82 | 3.14 |

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula I or formula II: 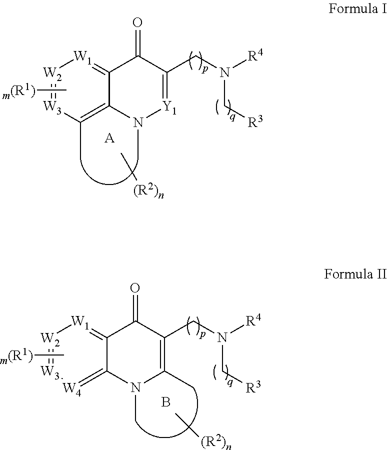
wherein
Ring A is a 5-, 6- or 7-membered substituted or unsubstituted heterocycle;
Ring B is a 4-, 5- or 6-membered substituted or unsubstituted heterocycle;
Y₁ is CR^{c} or N;
each of W₁, W₂, W₃ and W₄ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein R¹ is optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring, wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
R^{c} is H or C₁₋₂ alkyl, optionally substituted with 1-5 halo;
m is 0, 1, 2 or 3;
n is 0, 1, 2 or 3;
p is 1, 2 or 3; and
q is 1, 2 or 3;
or a pharmaceutically acceptable form or an isotope derivative thereof.

2. The compound of claim 1, having the structural formula I.

3. The compound of claim 2, wherein Ring A is selected from the group consisting of: 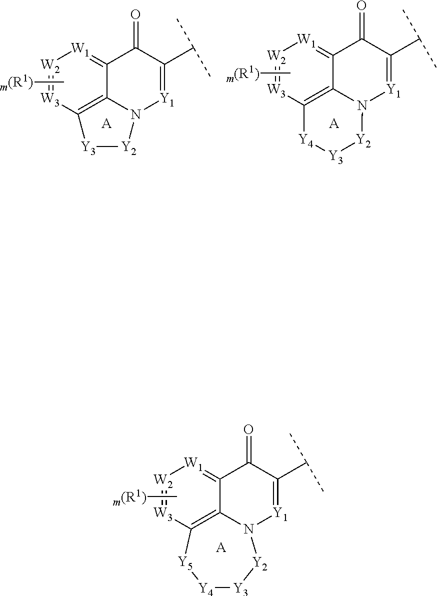
wherein:
Y₂ is CRR′ or C═O; and
each of Y₃, Y₄ and Y₅ is independently selected from N, O, S, CRR′ and C═O.

4. The compound of claim 3, wherein Ring A is selected from the group consisting of:

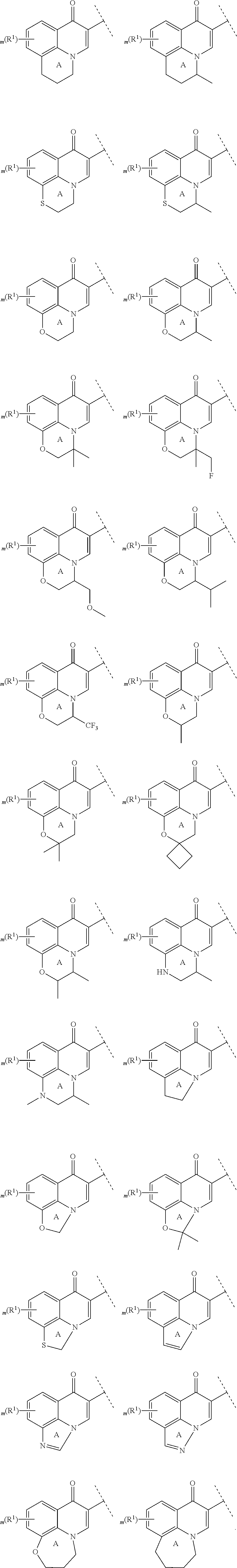

5. The compound of claim 1, having the structural formula II.

6. The compound of claim 5, wherein Ring B is selected from the group consisting of: 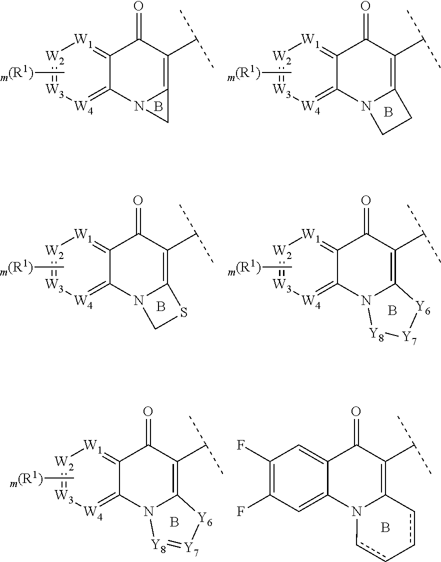
wherein:
Y₆ is CRR′ or S, O; and
each of Y₇ and Y₈ is independently N, O, or CRR′.

7. The compound of claim 5, wherein Ring B is selected from the group consisting of:

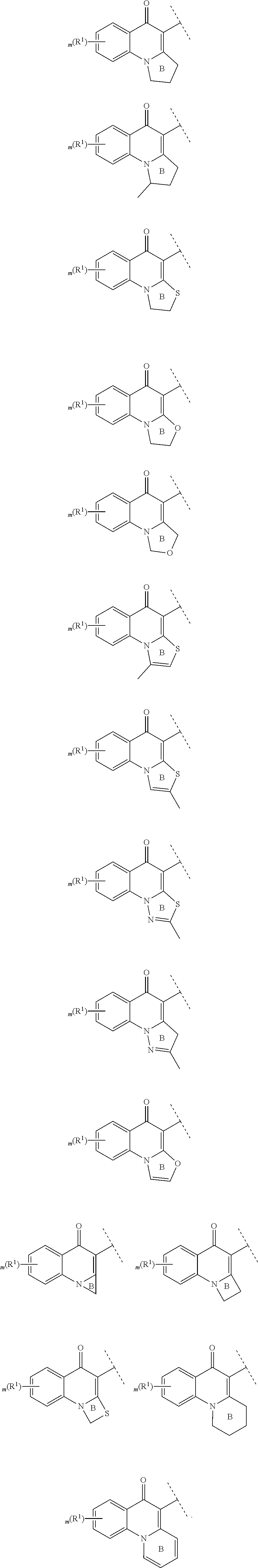

8. A compound having the structural formula III: 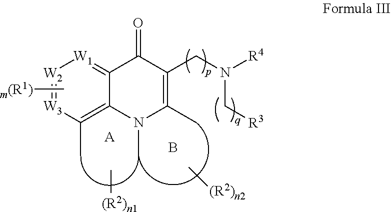
wherein
Ring A is a 5-, 6- or 7-membered substituted or unsubstituted heterocycle;
Ring B is a 4-, 5- or 6-membered substituted or unsubstituted heterocycle;
each of W₁, W₂, and W₃ is independently CH or N;
R¹ is selected from the group consisting of halo, oxo, OH, CN, OR, CF₃, C₁₋₆ alkyl, C₃₋₆ cycloalkyl, 3- to 6-membered heterocycloalkyl, 5- to 6-membered aryl or heteroaryl, 6- to 10-membered fused, spiro or bridged bicyclic ring, NRR′, N(R)C(═O)R, N(R)C(═O)(O)R, OC(═O)NRR′, C(═O)R, C(═O)NRR′, N(R)S(O)₂R, S(O)₂R and S(O)₂NRR′, wherein Riis optionally substituted with 1 to 4 same or different R^{a} or R^{b}; two R¹'s, along with carbon or heteroatoms they are attached to, may together form a 4- to 6-membered carbocyclic or heterocyclic ring;
R² is selected from the group consisting of halo, oxo, OH, CN, OR, CF₃ and C₁₋₆ alkyl, wherein aforementioned C₁₋₆ alkyl is optionally substituted with 1-2 R^{b}; and two R²'s along with carbon or heteroatoms they are attached to, may together form a 4- to 6-membered carbocyclic or heterocyclic ring;
R³ is a 5- to 6-membered aromatic carbocyclic or heterocyclic ring, a 9- to 10-membered aromatic carbocyclic or heterocyclic ring, wherein the aforementioned aromatic carbocyclic or heterocyclic rings is optionally substituted with 1 to 4 same or different R^{a};
R⁴ is S(═O)₂—C₁₋₄ alkenyl, S(═O)₂—C₁₋₄ alkyl, C(═O)—C₁₋₄ alkenyl, C(═O)—C₁₋₄ alkyl, C₁₋₄ alkenyl, C₁₋₄ alkyl, —C₁₋₄ alkyl-NH₂, —C₁₋₄ alkyl-OH, C₁₋₃ alkyl-NH—C₁₋₃ alkyl, C₁₋₃ alkyl-O—C₁₋₃ alkyl, C₁₋₃ alkyl-S—C₁₋₃ alkyl, C₁₋₃ alkyl-C(═O)—C₁₋₃ alkyl, C₁₋₃ alkyl-S(═O)₂—C₁₋₃ alkyl, or 3- to 6-membered saturated or unsaturated, carbocyclic or heterocyclic ring; wherein R⁴ is optionally substituted with 1 to 4 same or different R^{b};
each of R and R′ is independently H, or C₁₋₆ alkyl or C₃₋₆ cycloalkyl, optionally, R and R′, together with the nitrogen to which they are attached, form a 4- to 6-member ring, each optionally substituted with 1 to 3 substituents independently selected from the group consisting of C₁₋₃ alkyl, C₃₋₆ cycloalkyl or heterocyclic, halo, OH, OC₁₋₃ alkyl, and CN;
R^{a} is OH, NRR′, halogen, CN, NO₂, C₁₋₂ alkyl, C₁₋₂ haloalkyl, —C₁₋₄ alkyl-NRR′, —C₁₋₄ alkyl-OR, C₁₋₂ alkoxy, C(═O)NRR′, NRC(═O)R, or C(═O)R, wherein aforementioned alkyl, R or R′ is optionally substituted with 1-3 R^{b}, or two R^{a}'s form ═O, or two R^{a}'s together with the carbon atom to which they are bonded form a 3- to 5-membered saturated or unsaturated carbocyclic or heterocyclic ring;
R^{b} is halogen, CN, OH, C₁₋₂ alkyl, C₁₋₂ alkyl-OH, C₃₋₆ cycloalkyl, C₁₋₂ alkoxy, NRR′, S(═O)₂NRR′, C(═O)R, 5- to 6-membered aromatic carbocyclic or heterocyclic ring, C₁₋₂ alkyl-carbocyclyl, or C₁₋₂ alkyl-heterocyclyl, wherein the aforementioned alkyl, R, R′, aromatic carbocyclylic or heterocyclylic rings is optionally substituted with 1 to 4 same or different R^{a}; or two R^{b}'s form ═O; or two R^{b}'s attached to identical or neighboring carbon atoms may form a 3-membered carbocyclic ring;
m is 0, 1, 2 or 3;
n₁ is 0, 1, 2 or 3;
n₂ is 0, 1, 2 or 3;
p is 1, 2 or 3; and
q is 1, 2 or 3;
or a pharmaceutically acceptable form or an isotope derivative thereof.

9. The compound of claim 8, wherein Rings A-B is selected from the group consisting of:

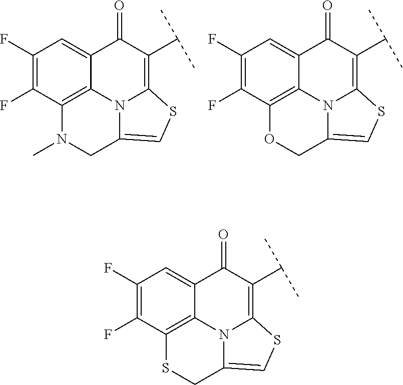

10. The compound of claim 9, wherein R³ is a 5- or 6-membered substituted or unsubstituted aromatic or heteroaromatic group.

11. The compound of claim 9, wherein R³ having a structure selected from the group consisting of:

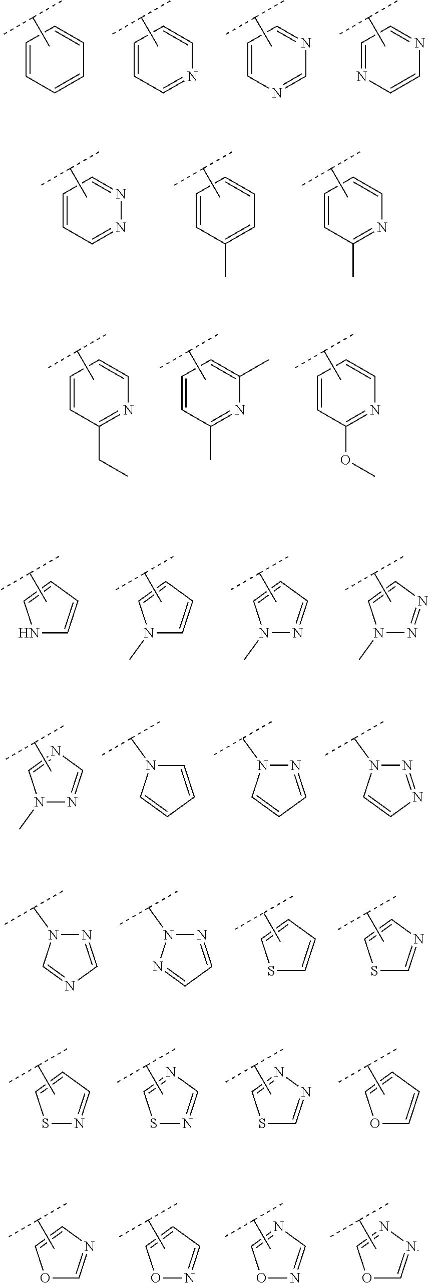

12. A compound selected from the group consisting of:

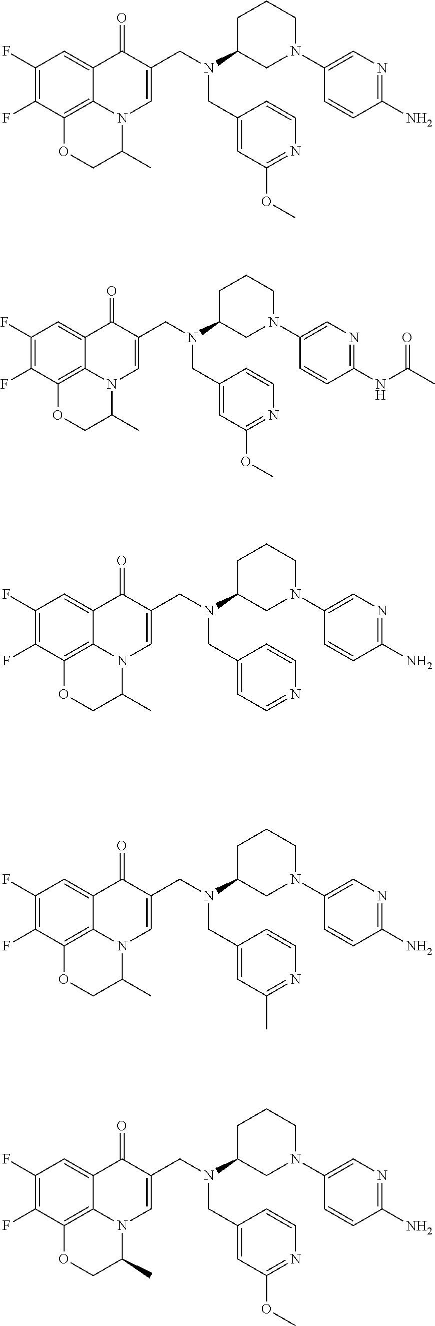 [SMILES: COc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3C)[C@H]2CCCN(c3ccc(NC(C)=O)nc3)C2)ccn1 ; CC1COc2c(F)c(F)cc3c(=O)c(CN(Cc4ccncc4)[C@H]4CCCN(c5ccc(N)nc5)C4)cn1c23 ; Cc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OC[C@@H]3C)[C@H]2CCCN(c3ccc(N)nc3)C2)ccn1]

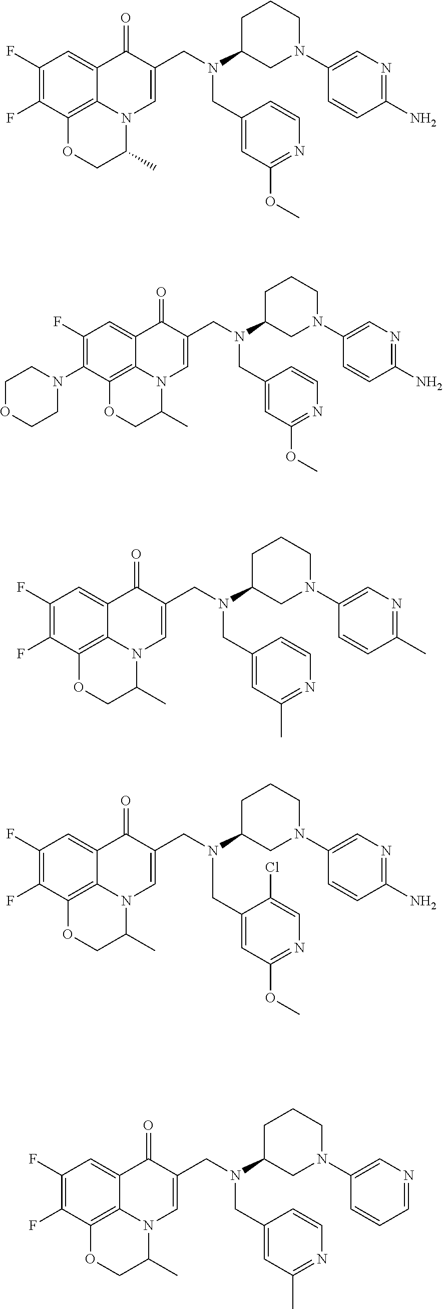 [SMILES: COc1cc(CN(Cc2cn3c4c(c(F)c(F)cc4c2=O)OC[C@H]3C)[C@H]2CCCN(c3ccc(N)nc3)C2)ccn1 ; COc1cc(CN(Cc2cn3c4c(c(N5CCOCC5)c(F)cc4c2=O)OCC3C)[C@H]2CCCN(c3ccc(N)nc3)C2)ccn1 ; Cc1ccc(N2CCC[C@H](N(Cc3ccnc(C)c3)Cc3cn4c5c(c(F)c(F)cc5c3=O)OCC4C)C2)cn1 ; COc1cc(CN(Cc2cn3c4c(c(F)c(F)cc4c2=O)OCC3C)[C@H]2CCCN(c3ccc(N)nc3)C2)c(Cl)cn1 ; Cc1cc(CN(Cc2cn3c4c(c(F)c(F)cc4c2=O)OCC3C)[C@H]2CCCN(c3cccnc3)C2)ccn1]

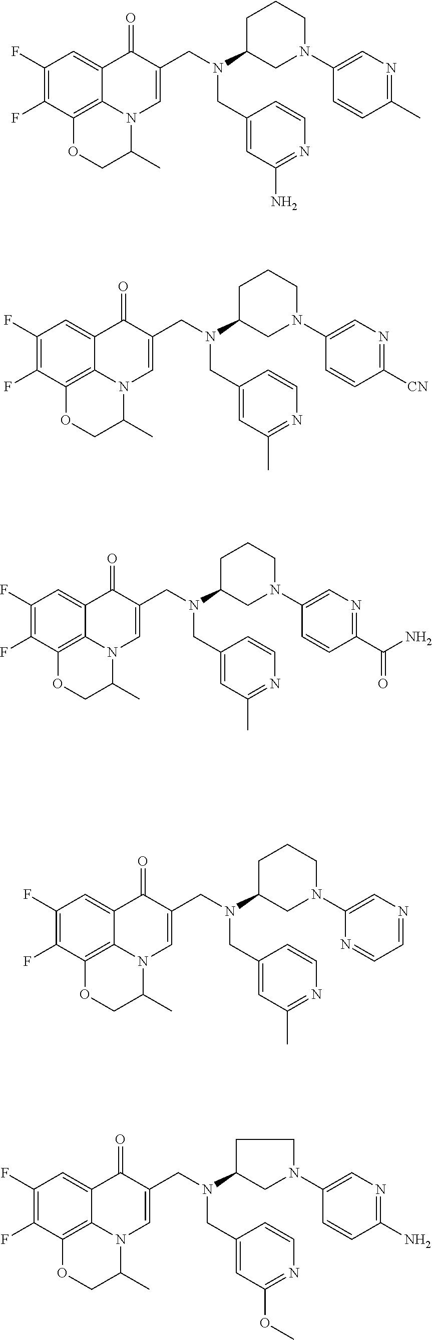 [SMILES: Cc1ccc(N2CCC[C@H](N(Cc3ccnc(N)c3)Cc3cn4c5c(c(F)c(F)cc5c3=O)OCC4C)C2)cn1 ; Cc1cc(CN(Cc2cn3c4c(c(F)c(F)cc4c2=O)OCC3C)[C@H]2CCCN(c3ccc(C#N)nc3)C2)ccn1 ; Cc1cc(CN(Cc2cn3c4c(c(F)c(F)cc4c2=O)OCC3C)[C@H]2CCCN(c3ccc(C(N)=O)nc3)C2)ccn1 ; Cc1cc(CN(Cc2cn3c4c(c(F)c(F)cc4c2=O)OCC3C)[C@H]2CCCN(c3cnccn3)C2)ccn1 ; COc1cc(CN(Cc2cn3c4c(c(F)c(F)cc4c2=O)OCC3C)[C@H]2CCN(c3ccc(N)nc3)C2)ccn1]

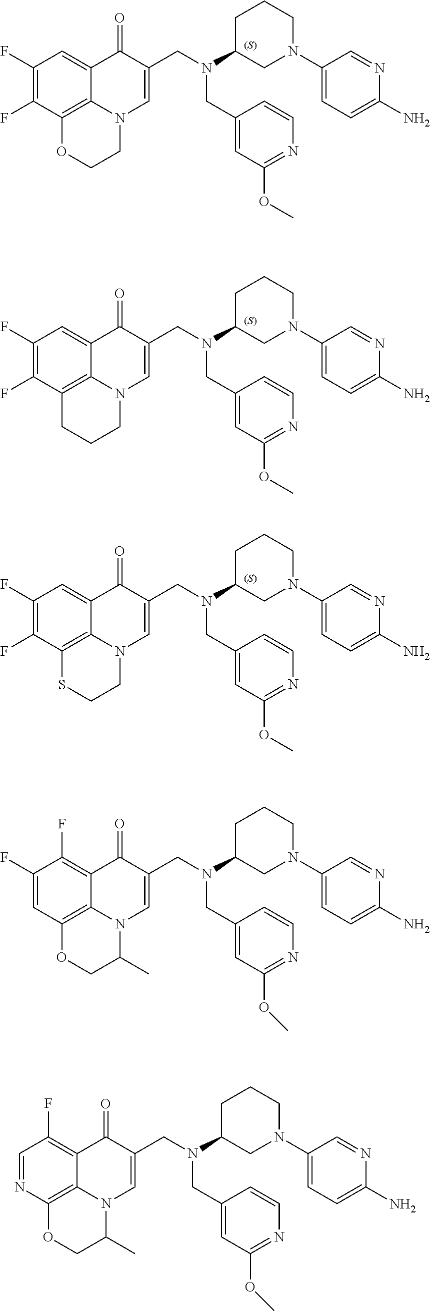

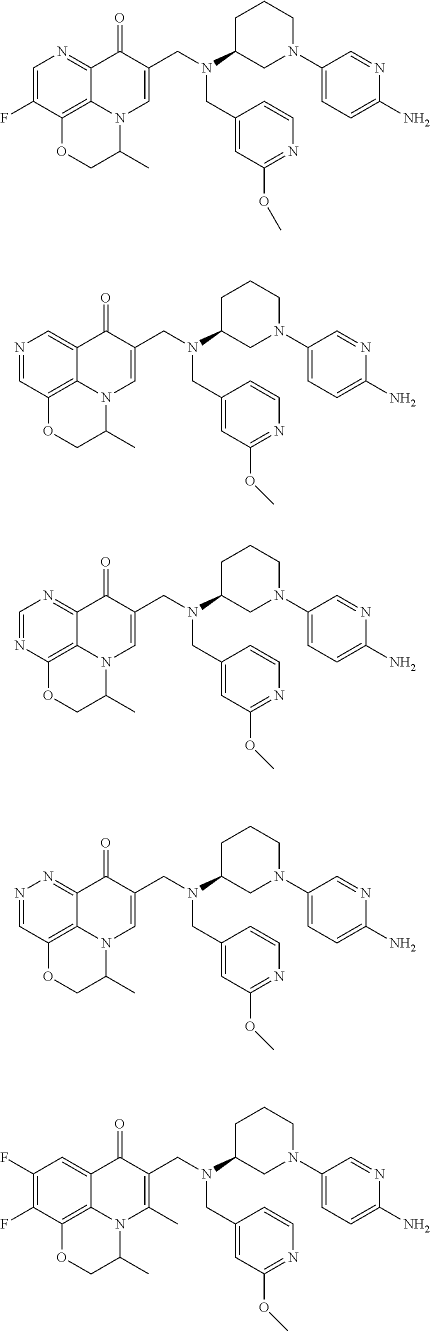 [SMILES: COc1cc(CN(Cc2cn3c4c(c(F)cnc4c2=O)OCC3C)[C@H]2CCCN(c3ccc(N)nc3)C2)ccn1 ; COc1cc(CN(Cc2cn3c4c(cncc4c2=O)OCC3C)[C@H]2CCCN(c3ccc(N)nc3)C2)ccn1 ; COc1cc(CN(Cc2cn3c4c(ncnc4c2=O)OCC3C)[C@H]2CCCN(c3ccc(N)nc3)C2)ccn1 ; COc1cc(CN(Cc2cn3c4c(cnnc4c2=O)OCC3C)[C@H]2CCCN(c3ccc(N)nc3)C2)ccn1 ; COc1cc(CN(Cc2c(C)n3c4c(c(F)c(F)cc4c2=O)OCC3C)[C@H]2CCCN(c3ccc(N)nc3)C2)ccn1]

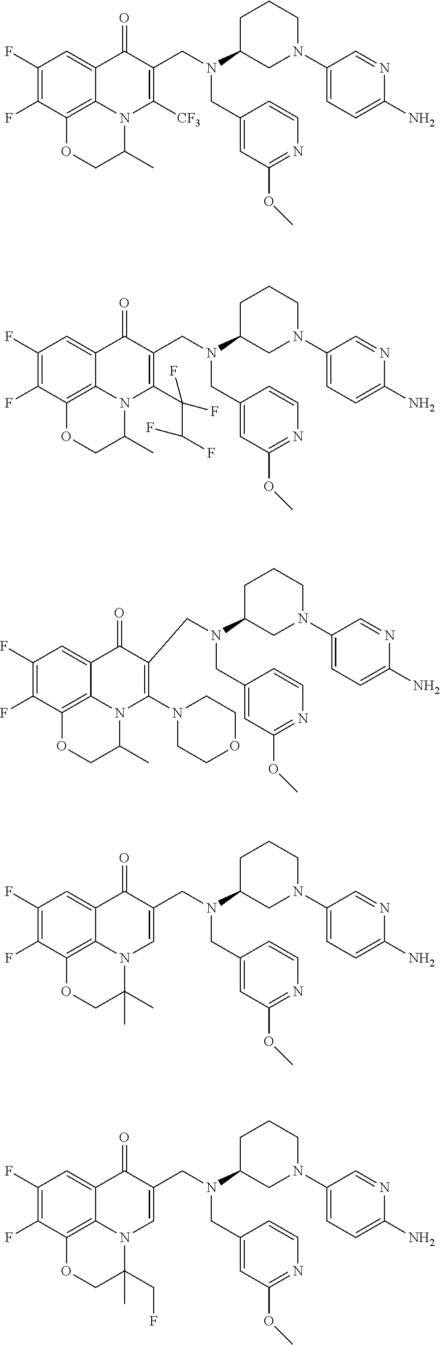 [SMILES: COc1cc(CN(Cc2c(C(F)(F)F)n3c4c(c(F)c(F)cc4c2=O)OCC3C)[C@H]2CCCN(c3ccc(N)nc3)C2)ccn1 ; COc1cc(CN(Cc2c(C(F)(F)C(F)F)n3c4c(c(F)c(F)cc4c2=O)OCC3C)[C@H]2CCCN(c3ccc(N)nc3)C2)ccn1 ; COc1cc(CN(Cc2c(N3CCOCC3)n3c4c(c(F)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3(C)C)[C@H]2CCCN(c3ccc(N)nc3)C2)ccn1 ; COc1cc(CN(Cc2cn3c4c(c(F)c(F)cc4c2=O)OCC3(C)CF)[C@H]2CCCN(c3ccc(N)nc3)C2)ccn1]

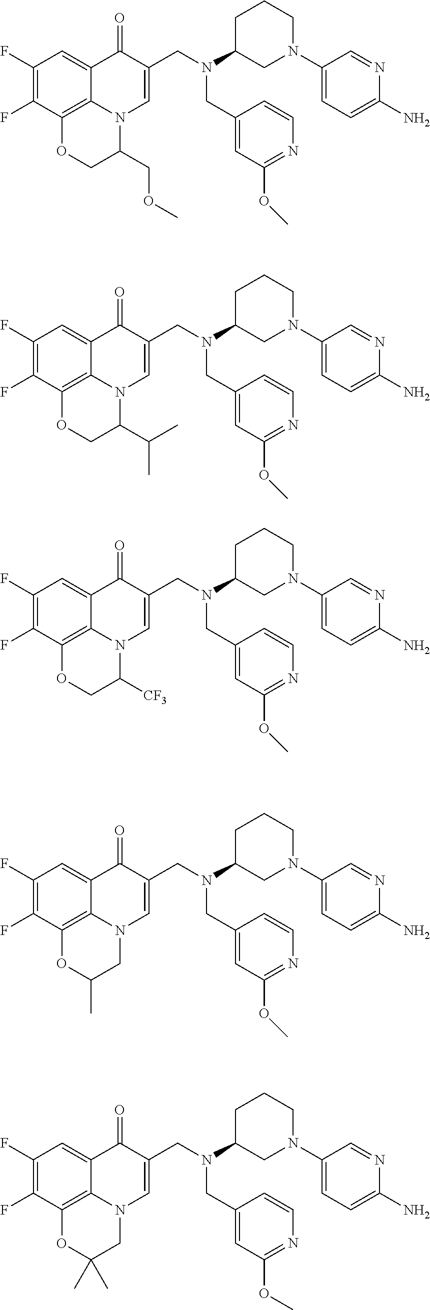

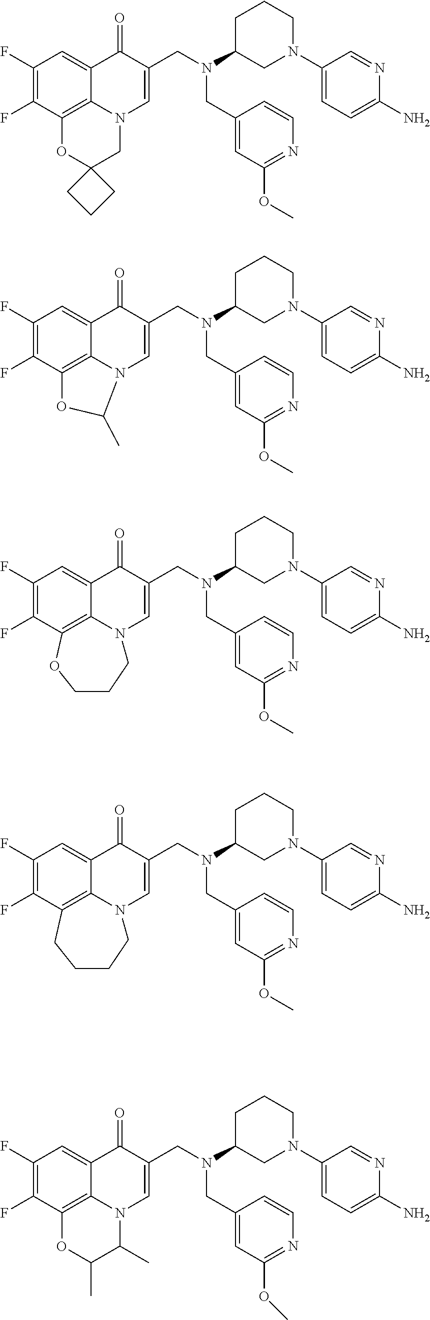 [SMILES: COc1cc(CN(Cc2cn3c4c(c(F)c(F)cc4c2=O)OC2(CCC2)C3)[C@H]2CCCN(c3ccc(N)nc3)C2)ccn1 ; COc1cc(CN(Cc2cn3c4c(c(F)c(F)cc4c2=O)OC3C)[C@H]2CCCN(c3ccc(N)nc3)C2)ccn1 ; COc1cc(CN(Cc2cn3c4c(c(F)c(F)cc4c2=O)OCCC3)[C@H]2CCCN(c3ccc(N)nc3)C2)ccn1 ; COc1cc(CN(Cc2cn3c4c(c(F)c(F)cc4c2=O)CCCC3)[C@H]2CCCN(c3ccc(N)nc3)C2)ccn1 ; COc1cc(CN(Cc2cn3c4c(c(F)c(F)cc4c2=O)OC(C)C3C)[C@H]2CCCN(c3ccc(N)nc3)C2)ccn1]

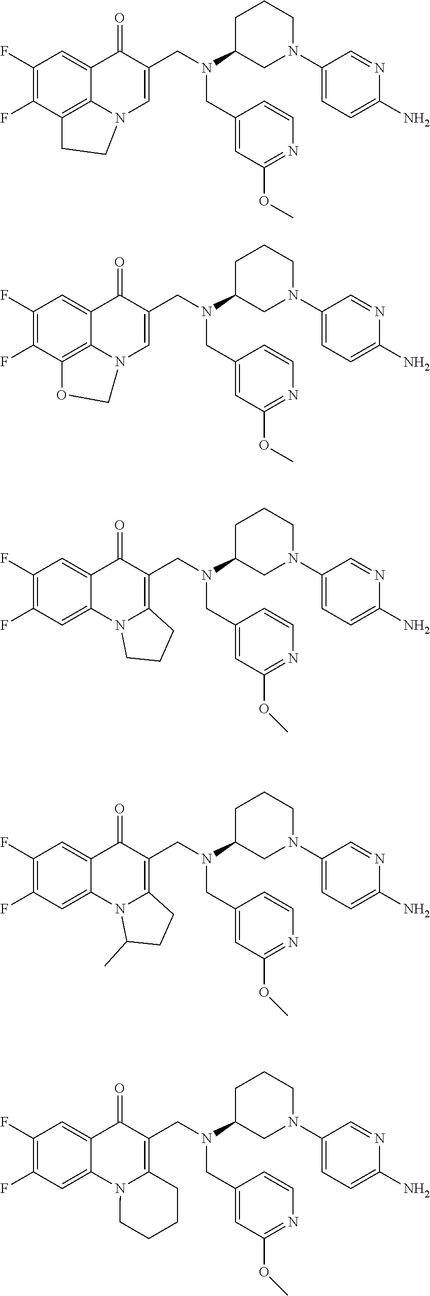

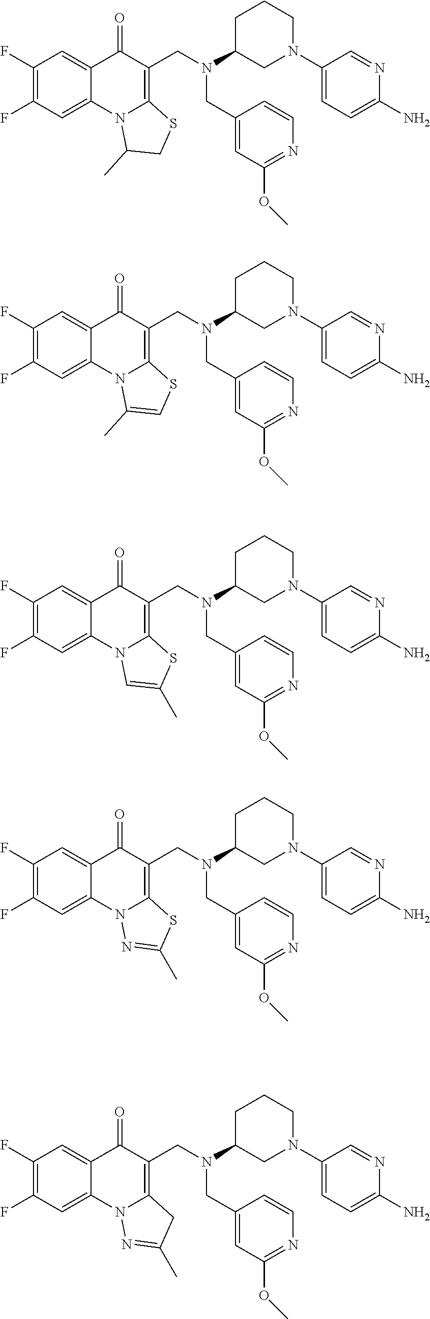

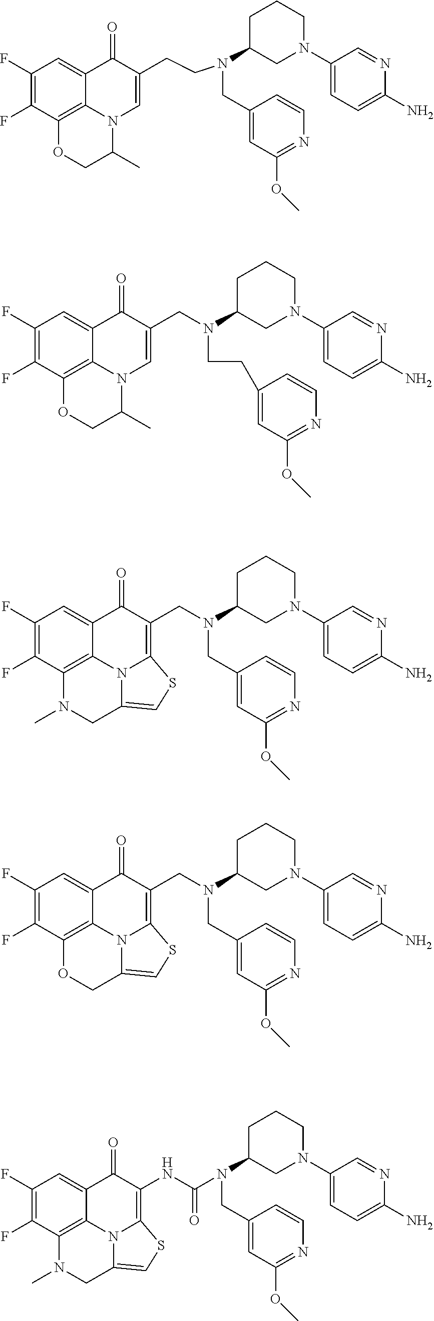 [SMILES: COc1cc(CN(CCc2cn3c4c(c(F)c(F)cc4c2=O)OCC3C)[C@H]2CCCN(c3ccc(N)nc3)C2)ccn1 ; COc1cc(CCN(Cc2cn3c4c(c(F)c(F)cc4c2=O)OCC3C)[C@H]2CCCN(c3ccc(N)nc3)C2)ccn1 ; COc1cc(CN(Cc2c(=O)c3cc(F)c(F)c4c3n3c(csc23)CN4C)[C@H]2CCCN(c3ccc(N)nc3)C2)ccn1 ; COc1cc(CN(Cc2c(=O)c3cc(F)c(F)c4c3n3c(csc23)CO4)[C@H]2CCCN(c3ccc(N)nc3)C2)ccn1 ; COc1cc(CN(C(=O)Nc2c(=O)c3cc(F)c(F)c4c3n3c(csc23)CN4C)[C@H]2CCCN(c3ccc(N)nc3)C2)ccn1]

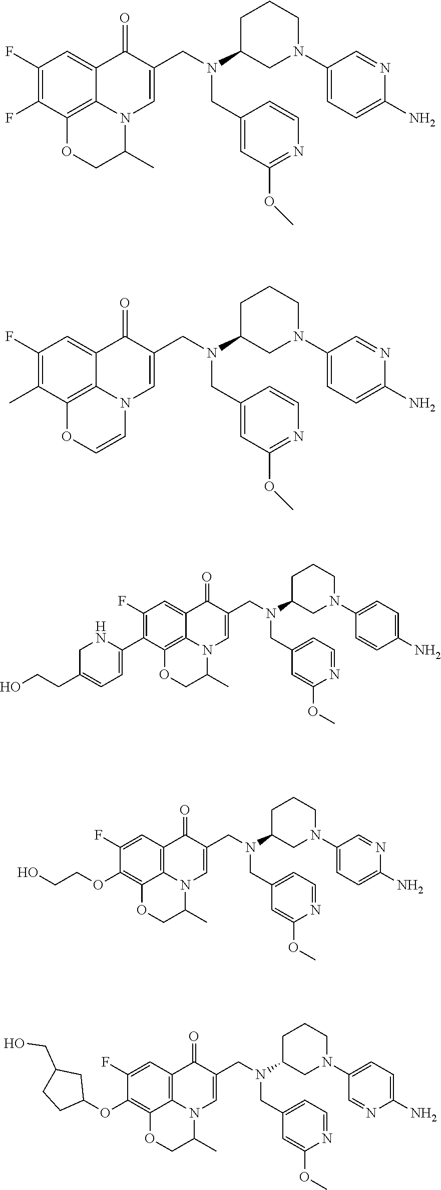 [SMILES: COc1cc(CN(Cc2cn3c4c(c(F)c(F)cc4c2=O)OCC3C)[C@H]2CCCN(c3ccc(N)nc3)C2)ccn1 ; COc1cc(CN(Cc2cn3c4c(c(C)c(F)cc4c2=O)OC=C3)[C@H]2CCCN(c3ccc(N)nc3)C2)ccn1 ; COc1cc(CN(Cc2cn3c4c(c(C5=CC=C(CCO)CN5)c(F)cc4c2=O)OCC3C)[C@H]2CCCN(c3ccc(N)cc3)C2)ccn1 ; COc1cc(CN(Cc2cn3c4c(c(OCCO)c(F)cc4c2=O)OCC3C)[C@H]2CCCN(c3ccc(N)nc3)C2)ccn1 ; COc1cc(CN(Cc2cn3c4c(c(OC5CCC(CO)C5)c(F)cc4c2=O)OCC3C)[C@@H]2CCCN(c3ccc(N)nc3)C2)ccn1]

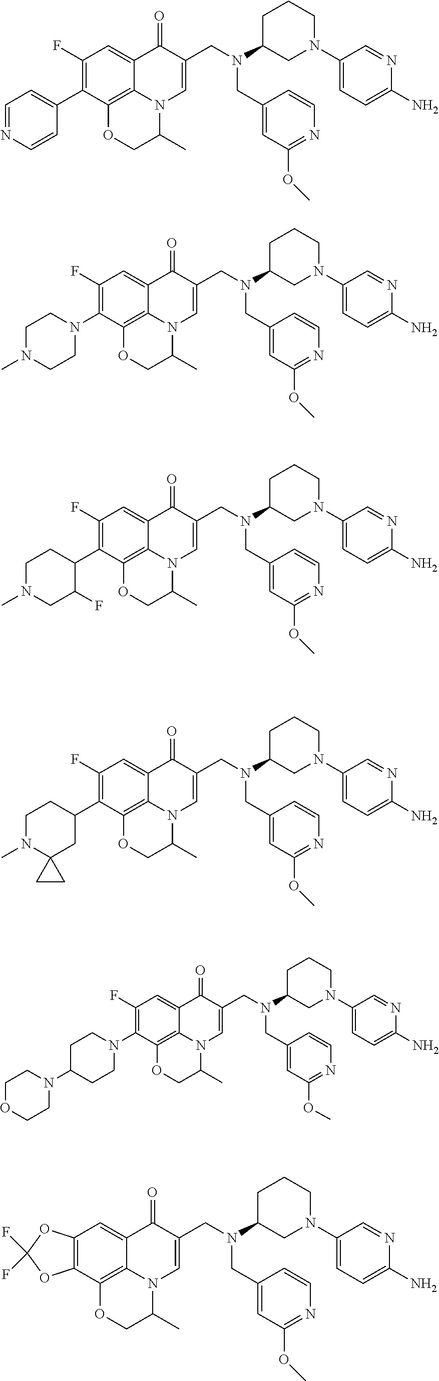

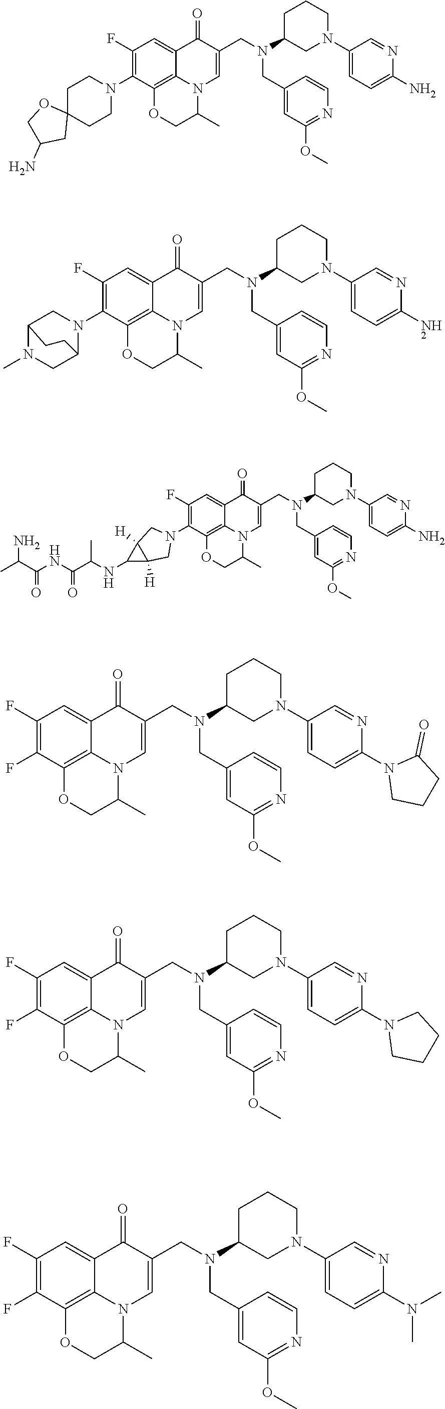

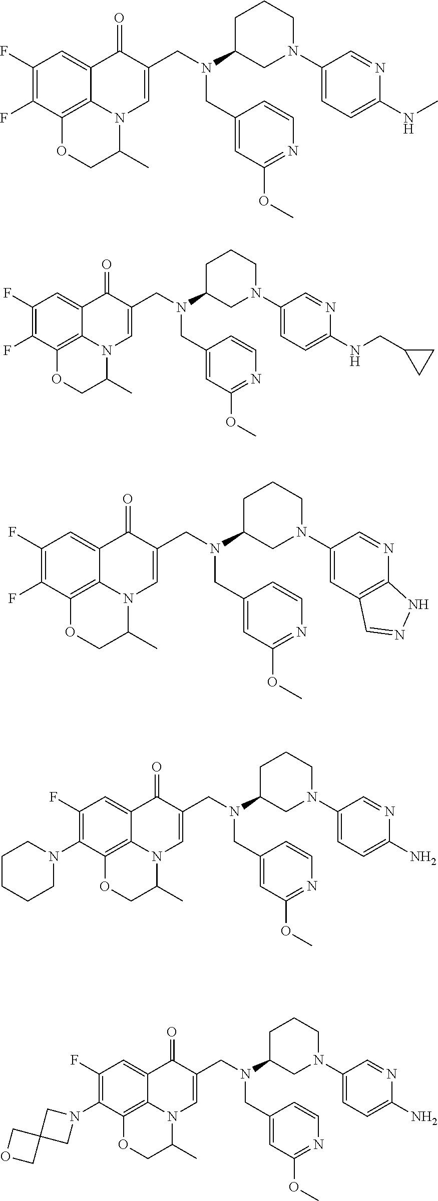 [SMILES: CNc1ccc(N2CCC[C@H](N(Cc3ccnc(OC)c3)Cc3cn4c5c(c(F)c(F)cc5c3=O)OCC4C)C2)cn1 ; COc1cc(CN(Cc2cn3c4c(c(F)c(F)cc4c2=O)OCC3C)[C@H]2CCCN(c3ccc(NCC4CC4)nc3)C2)ccn1 ; COc1cc(CN(Cc2cn3c4c(c(F)c(F)cc4c2=O)OCC3C)[C@H]2CCCN(c3cnc4[nH]ncc4c3)C2)ccn1 ; COc1cc(CN(Cc2cn3c4c(c(N5CCCCC5)c(F)cc4c2=O)OCC3C)[C@H]2CCCN(c3ccc(N)nc3)C2)ccn1 ; COc1cc(CN(Cc2cn3c4c(c(N5CC6(COC6)C5)c(F)cc4c2=O)OCC3C)[C@H]2CCCN(c3ccc(N)nc3)C2)ccn1]

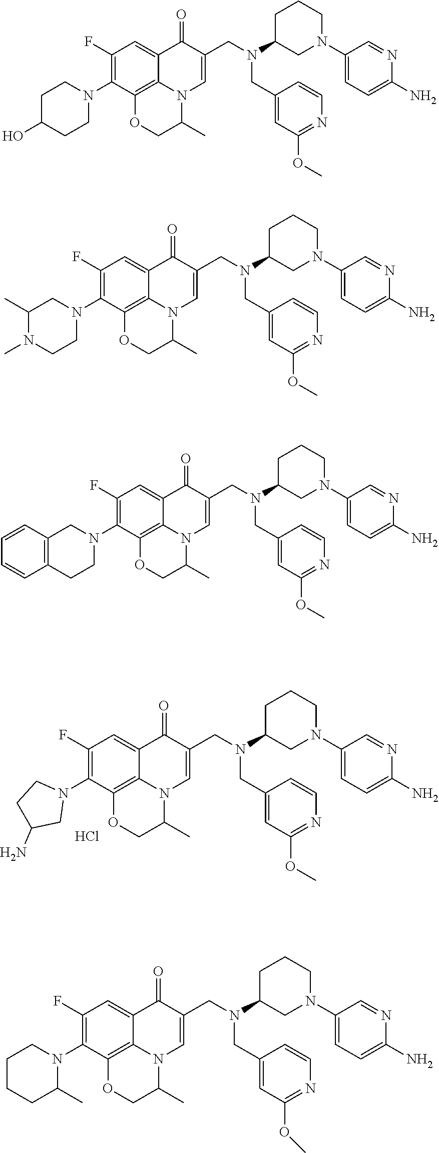 [SMILES: COc1cc(CN(Cc2cn3c4c(c(N5CCC(O)CC5)c(F)cc4c2=O)OCC3C)[C@H]2CCCN(c3ccc(N)nc3)C2)ccn1 ; COc1cc(CN(Cc2cn3c4c(c(N5CCN(C)C(C)C5)c(F)cc4c2=O)OCC3C)[C@H]2CCCN(c3ccc(N)nc3)C2)ccn1 ; COc1cc(CN(Cc2cn3c4c(c(N5CCc6ccccc6C5)c(F)cc4c2=O)OCC3C)[C@H]2CCCN(c3ccc(N)nc3)C2)ccn1 ; COc1cc(CN(Cc2cn3c4c(c(N5CCC(N)C5)c(F)cc4c2=O)OCC3C)[C@H]2CCCN(c3ccc(N)nc3)C2)ccn1 ; Cl ; COc1cc(CN(Cc2cn3c4c(c(N5CCCCC5C)c(F)cc4c2=O)OCC3C)[C@H]2CCCN(c3ccc(N)nc3)C2)ccn1]

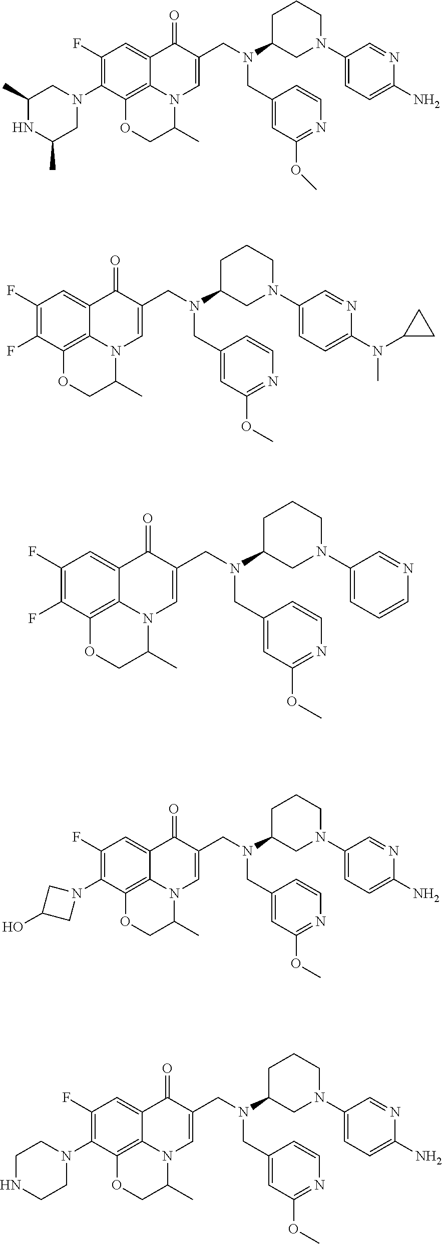 [SMILES: COc1cc(CN(Cc2cn3c4c(c(N5C[C@@H](C)N[C@@H](C)C5)c(F)cc4c2=O)OCC3C)[C@H]2CCCN(c3ccc(N)nc3)C2)ccn1 ; COc1cc(CN(Cc2cn3c4c(c(F)c(F)cc4c2=O)OCC3C)[C@H]2CCCN(c3ccc(N(C)C4CC4)nc3)C2)ccn1 ; COc1cc(CN(Cc2cn3c4c(c(F)c(F)cc4c2=O)OCC3C)[C@H]2CCCN(c3cccnc3)C2)ccn1 ; COc1cc(CN(Cc2cn3c4c(c(N5CC(O)C5)c(F)cc4c2=O)OCC3C)[C@H]2CCCN(c3ccc(N)nc3)C2)ccn1 ; COc1cc(CN(Cc2cn3c4c(c(N5CCNCC5)c(F)cc4c2=O)OCC3C)[C@H]2CCCN(c3ccc(N)nc3)C2)ccn1]

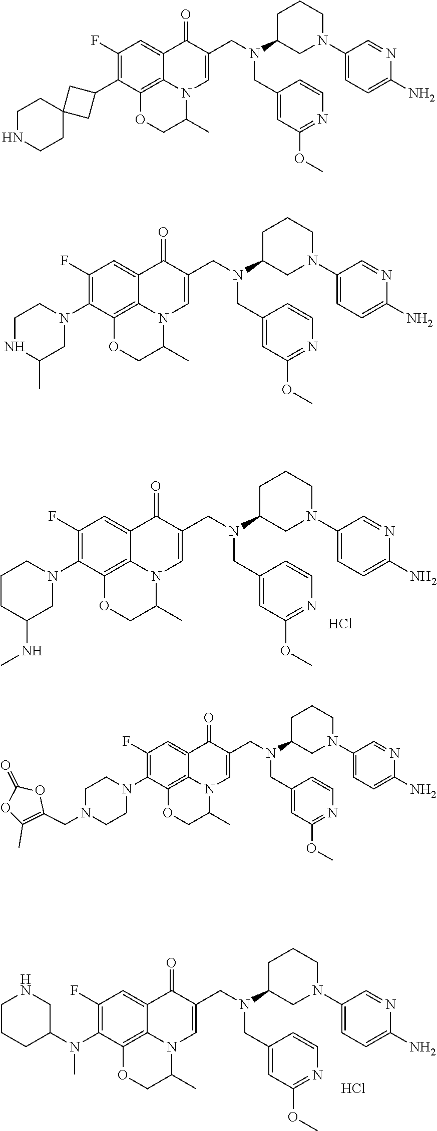

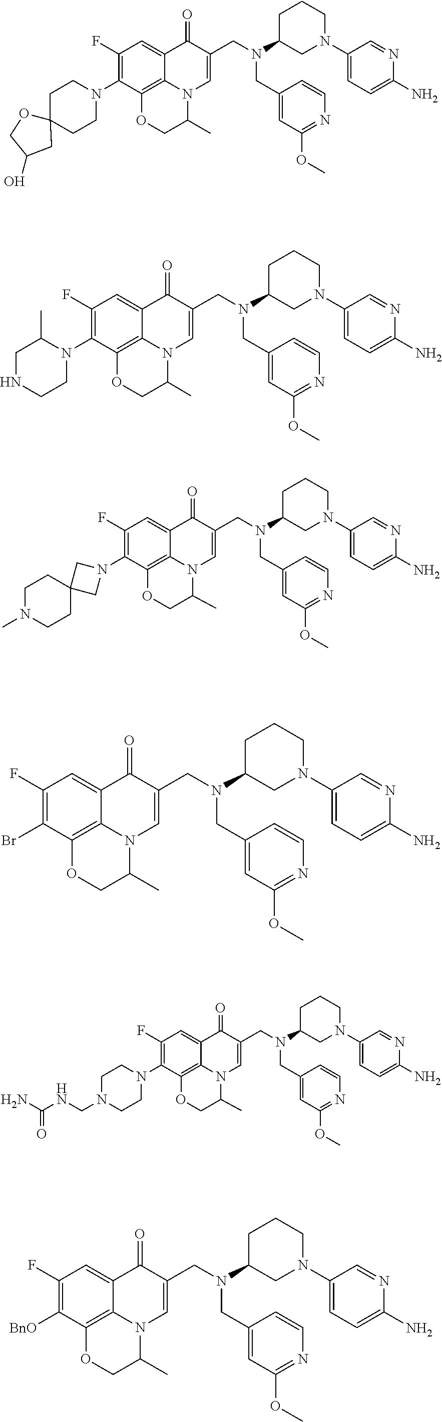

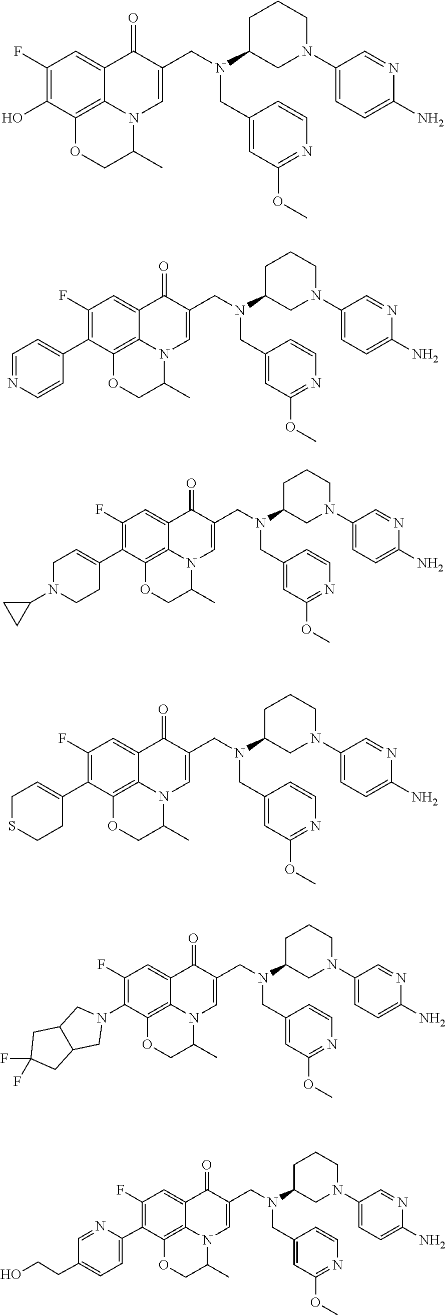

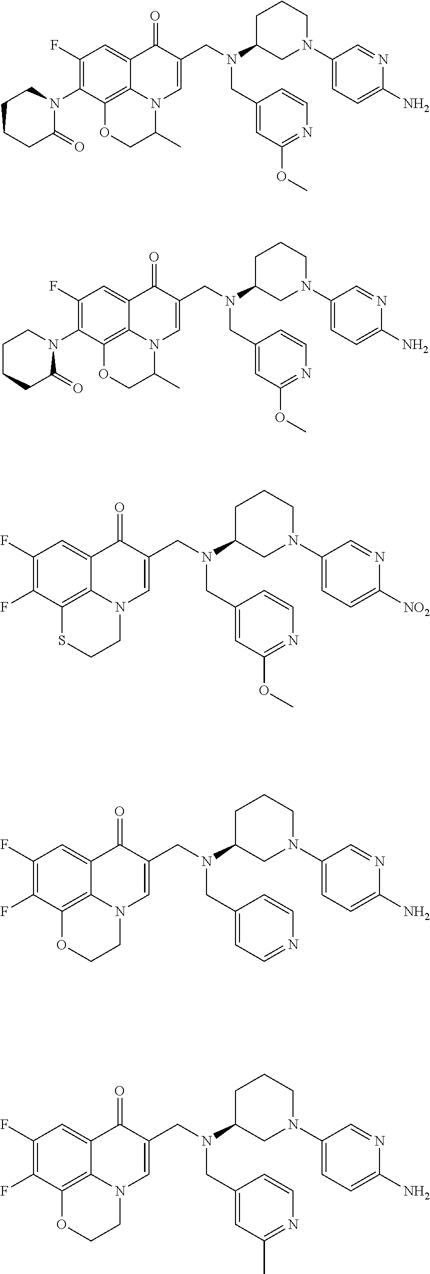

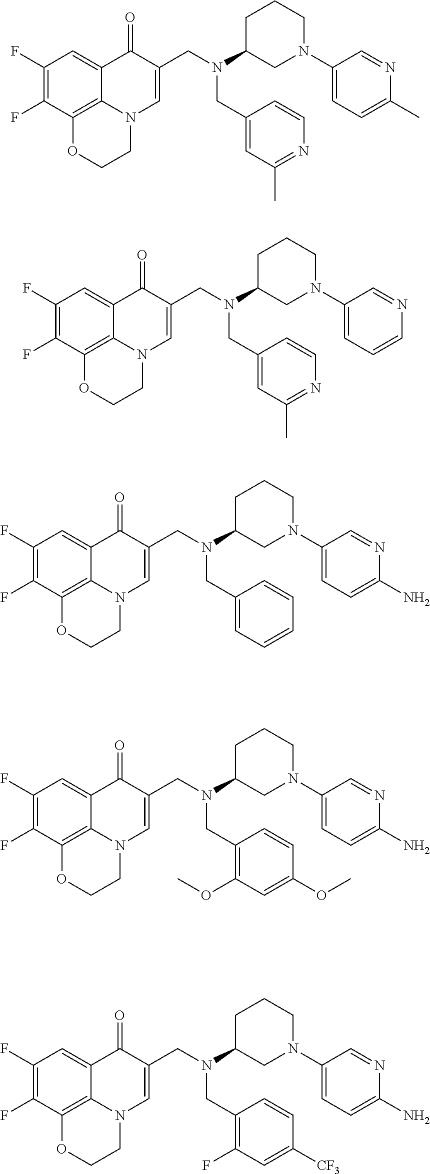

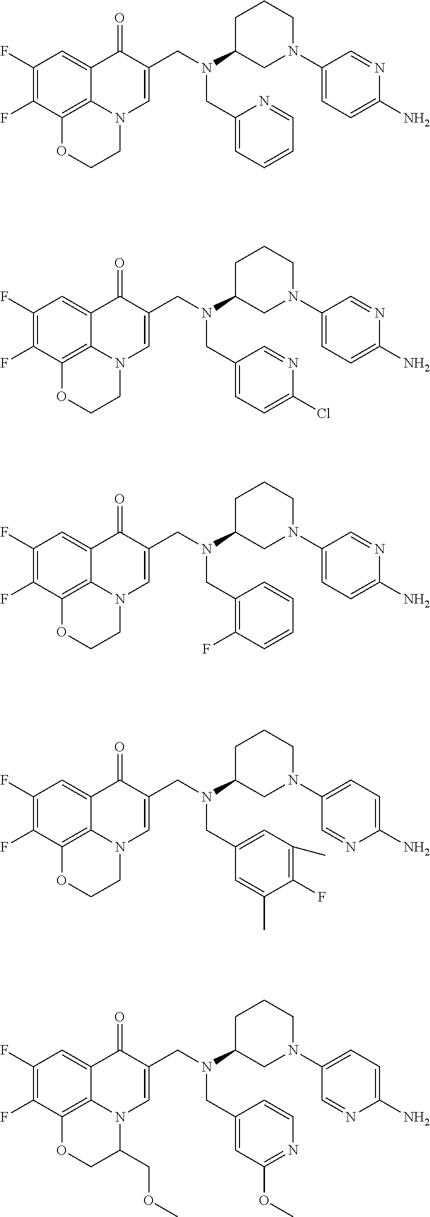 [SMILES: Nc1ccc(N2CCC[C@H](N(Cc3ccccn3)Cc3cn4c5c(c(F)c(F)cc5c3=O)OCC4)C2)cn1 ; Nc1ccc(N2CCC[C@H](N(Cc3ccc(Cl)nc3)Cc3cn4c5c(c(F)c(F)cc5c3=O)OCC4)C2)cn1 ; Nc1ccc(N2CCC[C@H](N(Cc3ccccc3F)Cc3cn4c5c(c(F)c(F)cc5c3=O)OCC4)C2)cn1 ; Cc1cc(CN(Cc2cn3c4c(c(F)c(F)cc4c2=O)OCC3)[C@H]2CCCN(c3ccc(N)nc3)C2)cc(C)c1F ; COCC1COc2c(F)c(F)cc3c(=O)c(CN(Cc4ccnc(OC)c4)[C@H]4CCCN(c5ccc(N)nc5)C4)cn1c23]

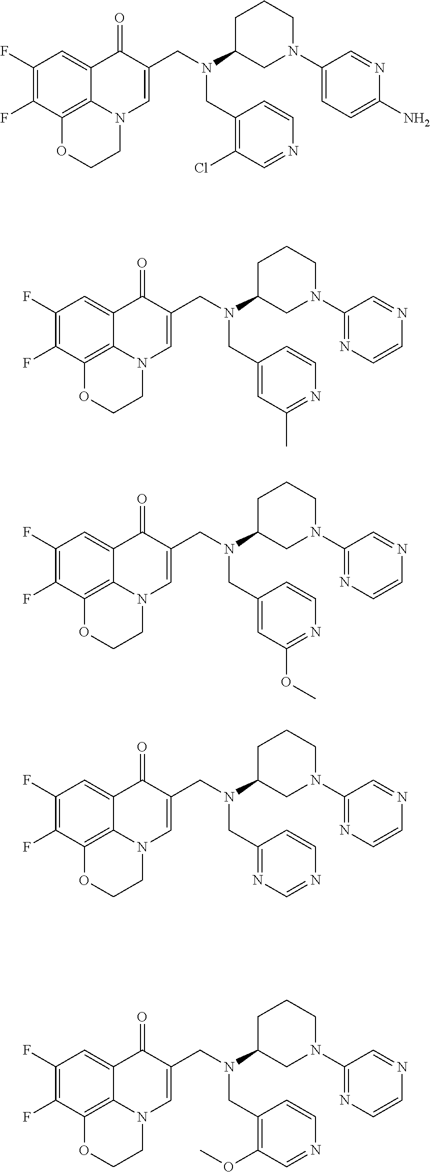

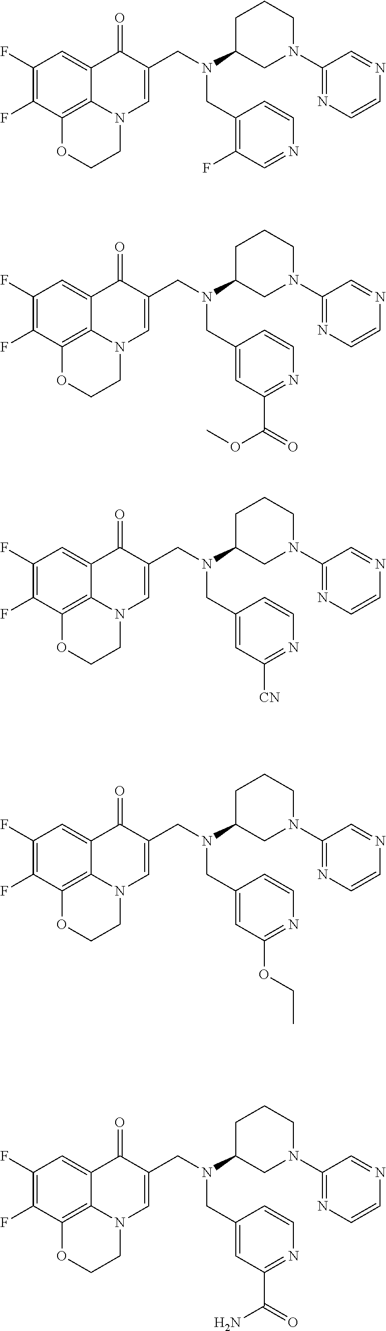 [SMILES: O=c1c(CN(Cc2ccncc2F)[C@H]2CCCN(c3cnccn3)C2)cn2c3c(c(F)c(F)cc13)OCC2 ; COC(=O)c1cc(CN(Cc2cn3c4c(c(F)c(F)cc4c2=O)OCC3)[C@H]2CCCN(c3cnccn3)C2)ccn1 ; N#Cc1cc(CN(Cc2cn3c4c(c(F)c(F)cc4c2=O)OCC3)[C@H]2CCCN(c3cnccn3)C2)ccn1 ; CCOc1cc(CN(Cc2cn3c4c(c(F)c(F)cc4c2=O)OCC3)[C@H]2CCCN(c3cnccn3)C2)ccn1 ; NC(=O)c1cc(CN(Cc2cn3c4c(c(F)c(F)cc4c2=O)OCC3)[C@H]2CCCN(c3cnccn3)C2)ccn1]

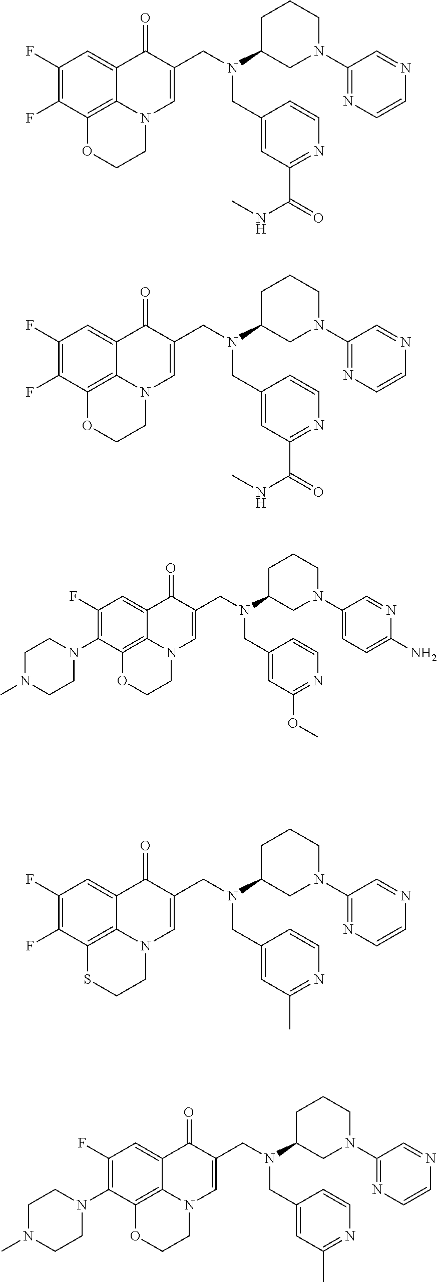 [SMILES: CNC(=O)c1cc(CN(Cc2cn3c4c(c(F)c(F)cc4c2=O)OCC3)[C@H]2CCCN(c3cnccn3)C2)ccn1 ; CNC(=O)c1cc(CN(Cc2cn3c4c(c(F)c(F)cc4c2=O)OCC3)[C@H]2CCCN(c3cnccn3)C2)ccn1 ; COc1cc(CN(Cc2cn3c4c(c(N5CCN(C)CC5)c(F)cc4c2=O)OCC3)[C@H]2CCCN(c3ccc(N)nc3)C2)ccn1 ; Cc1cc(CN(Cc2cn3c4c(c(F)c(F)cc4c2=O)SCC3)[C@H]2CCCN(c3cnccn3)C2)ccn1 ; Cc1cc(CN(Cc2cn3c4c(c(N5CCN(C)CC5)c(F)cc4c2=O)OCC3)[C@H]2CCCN(c3cnccn3)C2)ccn1]

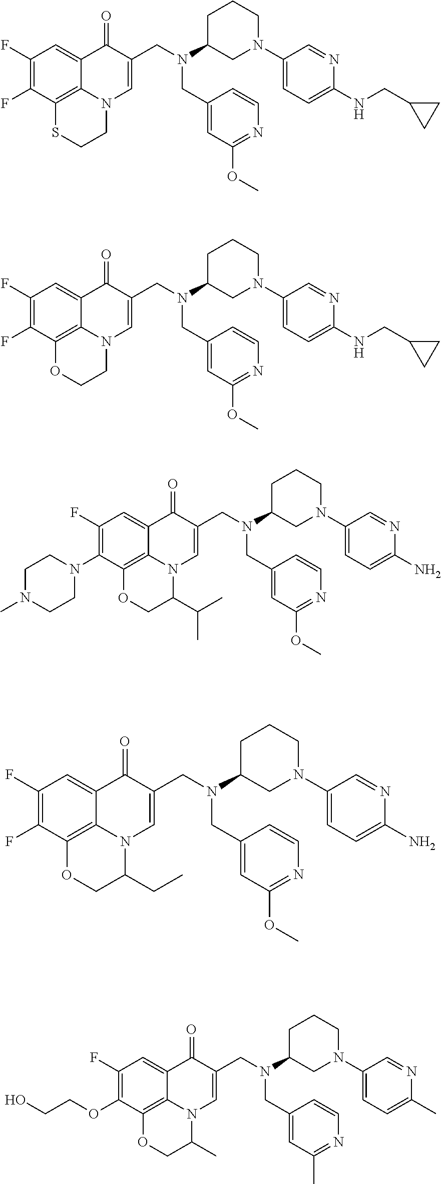 [SMILES: COc1cc(CN(Cc2cn3c4c(c(F)c(F)cc4c2=O)SCC3)[C@H]2CCCN(c3ccc(NCC4CC4)nc3)C2)ccn1 ; COc1cc(CN(Cc2cn3c4c(c(F)c(F)cc4c2=O)OCC3)[C@H]2CCCN(c3ccc(NCC4CC4)nc3)C2)ccn1 ; COc1cc(CN(Cc2cn3c4c(c(N5CCN(C)CC5)c(F)cc4c2=O)OCC3C(C)C)[C@H]2CCCN(c3ccc(N)nc3)C2)ccn1 ; CCC1COc2c(F)c(F)cc3c(=O)c(CN(Cc4ccnc(OC)c4)[C@H]4CCCN(c5ccc(N)nc5)C4)cn1c23 ; Cc1ccc(N2CCC[C@H](N(Cc3ccnc(C)c3)Cc3cn4c5c(c(OCCO)c(F)cc5c3=O)OCC4C)C2)cn1]

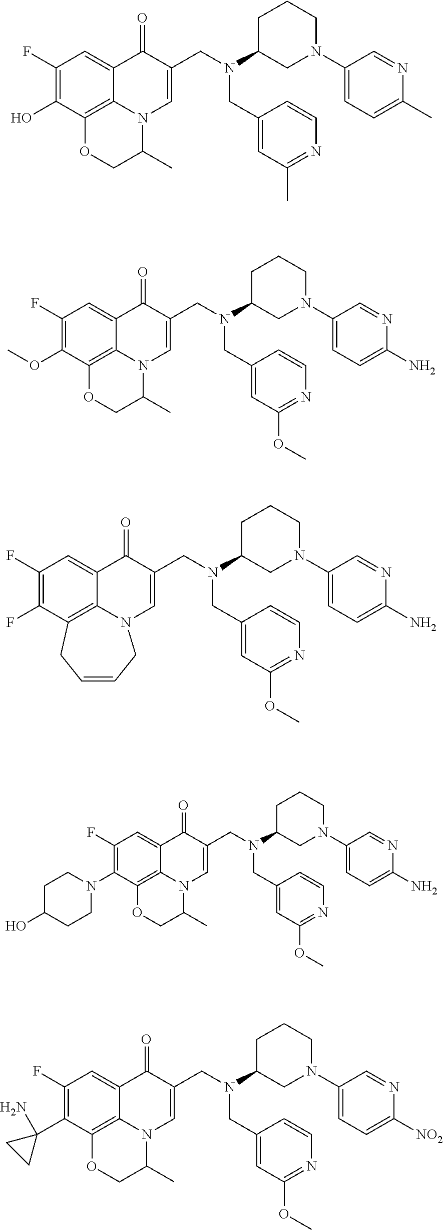

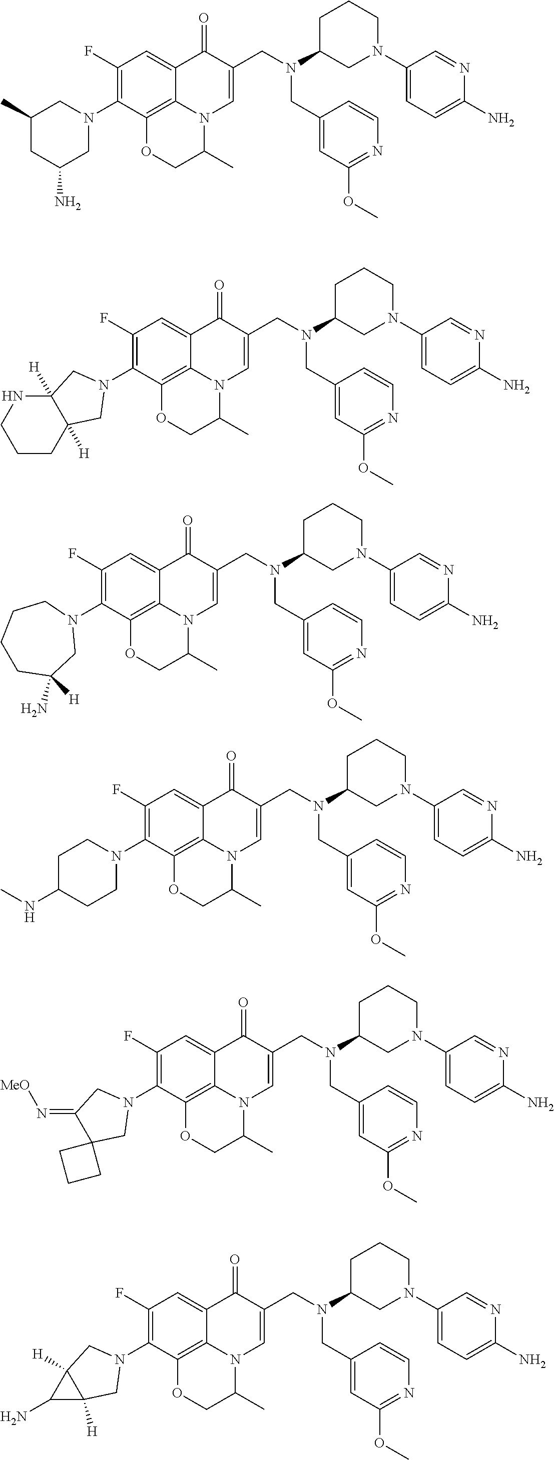

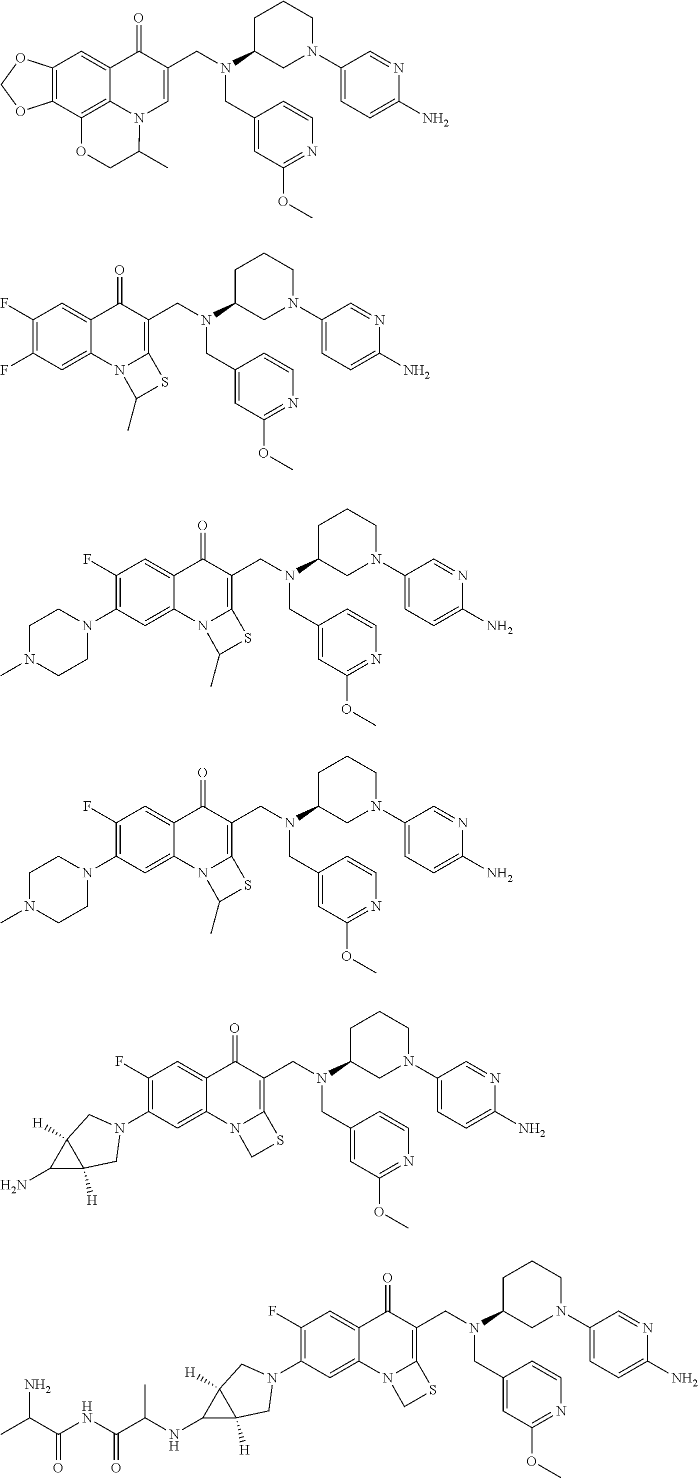

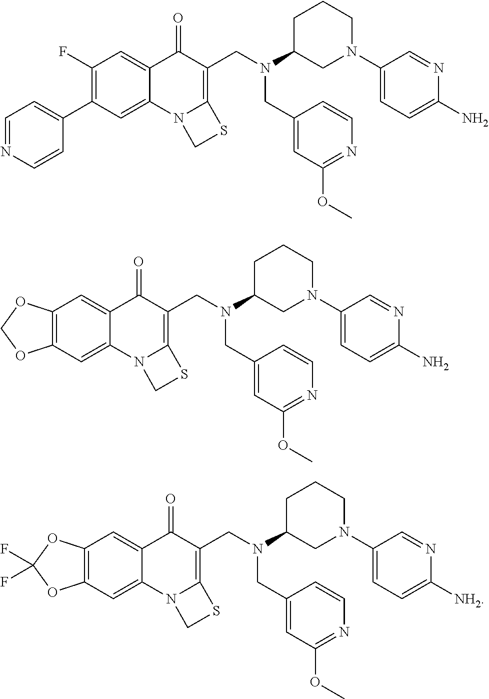

13. The compound of claim 12, having one or more deuterium atoms in place of one or more hydrogen atoms.

14. (canceled)

15. A pharmaceutical composition comprising a compound according to claim 1, effective to treat or reduce one or more diseases or disorders, in a mammal, including a human.

16. A unit dosage form comprising a pharmaceutical composition according to claim 15.

17. The unit dosage form of claim 16, being an injectable, a solution, a suspension, a tablet or a capsule.

18. A method for treating or reducing a disease or disorder mediated by or associated with STING, comprising administering to a subject in need thereof a pharmaceutical composition comprising a compound of claim 1.

19-22. (canceled)

23. A method for treating or reducing the effect of aging comprising administering to a subject in need thereof a pharmaceutical composition comprising a compound of claim 1.

24-27. (canceled)
